(19) United States
(12) Patent Application Publication
(10) Pub. No.: US 2026/0274832 A1
(43) Pub. Date: Sep. 17, 2026

(54) COMPOUNDS AND THEIR USE IN TREATING CANCER

(71) Applicant: ASTRAZENECA AB, Sodertalje (SE)

(72) Inventors: Peter ASTLES, Cambridge (GB); James SCOTT, Cambridge (GB)

(21) Appl. No.: 19/160,888

(22) PCT Filed: Mar. 8, 2024
(86) PCT No.: PCT/IB2024/052251

§ 371 Date: Aug. 29, 2025

Related U.S. Application Data
(60) Provisional application No. 63/489,528, filed on Mar. 10, 2023.

(51) Int. Cl. C07D 413/14; A61K 31/4545; A61K 31/496; A61K 31/497; A61K 31/501; A61K 31/506; A61K 31/513; A61K 31/5377; A61K 47/55; C07D 401/14; C07D 471/04; C07D 487/04

(52) U.S. Cl. CPC C07D 413/14; A61K 31/4545; A61K 31/496; A61K 31/497; A61K 31/501; A61K 31/506; A61K 31/513; A61K 31/5377; A61K 47/55; C07D 401/14; C07D 471/04; C07D 487/04

(57) ABSTRACT

A compound of Formula (I): or a pharmaceutically acceptable salt thereof, wherein: X¹ is CH or N; p is 0, 1 or 2; where: each R¹ is a substituent on any C atom and is independently selected from F, Cl, C₁₋₃alkyl and C₁₋₃ alkoxy, wherein said C₁₋₃alkyl and C₁₋₃alkoxy may be independently optionally substituted by one or more F; R^{N} is selected from H and Me; n is 0, 1 or 2; m is 0 or 1; Q¹ is CH or N; when n & m are both other than 0, Q² is CH or N; when n & m are both 0, Q² is CH; when n is 0 or 1, Q³ is CH; when n is 2 and Q² is N, Q³ is CH; when n is 2 and Q² is CH, Q³ is CH or 0; R^{2a} and R^{2b} are substituents on the same or different C atoms other than at Q¹ or Q², each independently selected from H, F and C₁₋₃alkyl, or R^{2a} & R^{2b} together form a —(CH₂)ᵣ group where r is 1, 2 or 3; Q⁴ is a single bond or —NR⁴C(═O); R⁴ is H or Me; 0, 1 or 2 of Y¹, Y², Y³, Y⁴ & Y⁵ is/are N, and are otherwise C; each R³ is a substituent on any C atom at Y¹, Y², Y³, Y⁴ & Y⁵, and is independently selected from F, Cl, CN, C₁₋₃alkyl and C₁₋₃ alkoxy, wherein said C₁₋₃ alkyl and C₁₋₃ alkoxy may be independently optionally substituted by one or more F; q is 0, 1 or 2; wherein Linker is attached at any available C atom at Y⁴ & Y⁵; Linker is a saturated or a partially or fully unsaturated framework comprising C and H atoms and at least one heteroatom, wherein said framework has end points of attachment ‘a’ and ‘b’ and a length of from 5 to 26 atoms between ‘a’ and ‘b’; wherein said framework may include one or more straight and/or branched chains and/or rings and is optionally substituted on any available C atom(s) by one or more F; and W is an E3 ubiquitin ligase cereblon binder unit.

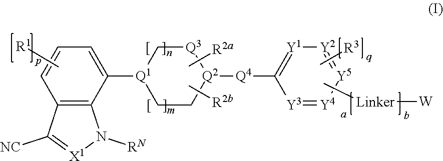

### RELATED APPLICATIONS

This application claims priority from U.S. Provisional Patent Application No. 63/489,528 filed Mar. 10, 2023, the disclosure of which is incorporated herein in its entirety.

### FIELD

This specification relates to certain Proteolysis Targeting Chimera (PROTAC) compounds and, as a minimum, their ability to degrade the Androgen Receptor (AR) and therefore their use for the treatment of diseases or disorders dependent on the androgen receptor in mammals. Degradation of androgen receptors may provide, for example, an anti-tumour effect and accordingly this specification relates in part to the use of such compounds for the treatment of cancer and to pharmaceutical compositions containing them. It also relates to intermediate compounds that may be useful in the preparation of such PROTACs.

### BACKGROUND

Traditional small molecule drugs reversibly (or sometimes irreversibly) bind to a target protein as a means of modulating a given biological activity. In contrast, PROTACs bind to their target proteins, but then bring about the target protein's degradation. Having achieved this effect, the PROTAC is in theory able to repeat this process with another target protein. Accordingly, unlike with “traditional small molecule” inhibitors, the PROTAC-driven degradation mechanism can in theory operate in a sub-stoichiometric manner—meaning that more modest exposures of a PROTAC compound could still achieve a desired level of efficacy in vivo. In practice this can mean that the degradation power (DC₅₀ and Dₘₐₓ) of a PROTAC can have an improved effect than that reflected only by its binding affinity.

At a simplistic level, PROTAC molecules are often described as having three parts—(1) a part that is capable of binding to the target protein to be degraded, (2) a second part that is capable of binding to an E3 ubiquitin ligase, and finally, a linker that connects (1) and (2) together.

In use, the PROTAC binds to both the target protein and E3 ubiquitin ligase simultaneously to form a ternary complex. The E3 ligase then recruits an E2 conjugating enzyme to the ternary complex, which ubiquitinates the target protein. This has the effect of labelling the target protein for degradation by the cell's proteasome machinery. A PROTAC can then dissociate from the target protein and initiate another cycle of this process in a catalytic manner. Meanwhile, the ubiquitinated target proteins are recognized and degraded by the cell's proteasome machinery.

This PROTAC-mediated approach may be valuable as a method of treating certain diseases where the targeted degradation of specific bodily proteins may be beneficial, for example in the treatment of cancer. One such cancer-related target is the androgen receptor.

The androgen receptor (AR) belongs to the steroid hormone group of nuclear receptors and is a ligand-dependent transcription factor which controls the expression of a range of genes involved in growth and survival of prostate cells. AR is composed of four distinct domains: the N-terminal domain (NTD), DNA binding domain (DBD), a hinge region which allows the N- and C-termini to interact and a C-terminal ligand binding domain (LBD). Androgens such as testosterone and its derivative dihydrotestosterone (DHT) bind to the AR ligand binding domain which releases AR chaperone proteins allowing AR to dimerise and translocate from the cytoplasm into the nucleus. Within the nucleus receptor dimers bind to androgen response elements (AREs) in the promoters of androgen responsive genes such as PSA and FKBP5. The AR signalling pathway plays a role in normal prostate development and male sexual differentiation fails to occur in the absence of androgens or without a functioning AR.

The relationship between androgens and prostate cancer was first discovered in a seminal study by Huggins and Hodges in 1941 and subsequent work has shown that androgen deprivation therapy is highly effective in the treatment of recurrent prostate cancer. However, despite good responses initially most tumour cells adapt to low androgen levels and patients relapse within a few years developing a disease state known as castrate resistant prostate cancer (CRPC). Several second generation antiandrogens have been approved by the US FDA for the treatment of CRPC. These include enzalutamide, apalutamide and darolutamide, which all compete with androgens for binding to the ligand binding domain. As well as antagonising the AR, preventing its activation, they also inhibit nuclear translocation and DNA binding thus effectively shutting down AR signalling. In contrast abiraterone acetate is an androgen biosynthesis inhibitor which targets cytochrome p450 enzyme 17R-hydroxlase-17,20-lyase (CYP17). Testosterone is processed in the testes and adrenal glands by CYP17 and therefore inhibition of this enzyme inhibits prostate tumour growth by decreasing circulating androgen levels.

Whilst the above-mentioned drugs significantly prolong survival of patients with late stage prostate cancer they are not curative and resistance inevitably occurs. However, it is clear that the AR remains central to the progression of CRPC meaning there is still a need to develop alternative AR inhibitors. Several resistance mechanisms have been identified including AR amplification, mutation or the generation of splice variants that lack the ligand binding domain. Mutations in the ligand binding domain, for example F877L or L702H, can convert antagonists into agonists or allow the receptor to utilise alternative steroid hormones such as glucocorticoids or progesterone. Agents that degrade AR, removing it from the cell could therefore help tackle these forms of resistance. As such, AR PROTACs, which bind the ligand binding domain of the androgen receptor and simultaneously recruit an E3 ligase such as cereblon leading to ubiquitination and degradation of AR via the proteasome, could offer therapeutic benefit to patients with prostate cancer, particularly metastatic CRPC. AR PROTACs may also be useful against AR+ breast cancer.

Given that the above-mentioned resistance mutations that can occur in the ligand binding domain and may limit the effectiveness of known CRPC treatments, it would be beneficial to develop AR degrading PROTACs that do not just target the “wild-type” AR, but which are also effective in degrading clinically relevant mutated forms of AR too.

Whichever target protein binding unit (1) is used at one end of a PROTAC's linker unit, a fundamental element that must always be present at the other end of a PROTAC molecule is a unit (2) that helps to direct the tagging of the target protein for degradation, for example an E3 ubiquitin ligase cereblon binder unit. Scientific endeavours have already provided a number of such E3 ubiquitin ligase cereblon binder units and further examples are demonstrated hereinafter by the present researchers.

WO2018/071606 describes certain PROTAC compounds said to be AR degraders.

As with “traditional small molecule” binders and PROTACs alike, there is always the issue of “off-target” activity in vivo which can be important to avoid in the development of safe and effective drug treatments. In other words, a given binding unit may be very potent against the intended target, but if it is inadvertently potent against other unintended biological targets in the human body, it may cause unacceptable toxicities, side effects and so on.

It is therefore an ongoing challenge to develop potent molecules for pharmaceutical use that are also suitably selective—i.e. avoiding inhibition/binding/degradation of unintended biological targets in vivo.

For example, the present researchers have surprisingly found that certain compounds of this specification show beneficial selectivity that is expected to avoid or reduce the risk of mitotoxicity in vivo.

As part of developing current and future PROTAC drug treatments for medicinal use (e.g. cancer), there is still a need to develop androgen receptor PROTAC compounds that have a combination of beneficial/improved properties. As mentioned above, development of PROTACs that target both wild-type AR and one or more clinically relevant mutated forms of AR is an example of such a combination effect.

Furthermore, there is a need to develop androgen receptor binder units that can be incorporated into a PROTAC, regardless of which an E3 ubiquitin ligase cereblon binder unit is selected for use at the other end of the molecule.

Other properties of interest during pharmaceutical discovery and development of such PROTACs may relate to selectivity profile, absorption/bioavailability, distribution, metabolism, elimination, toxicity and side-effect profile, stability, manufacturability and so on.

### SUMMARY

The compounds of this specification provide, as a minimum, potent AR binding units suitable for incorporation into PROTAC compounds, and to PROTAC compounds containing such AR binding units together with an E3 ubiquitin ligase cereblon binder unit at the other end of the PROTAC molecule. Certain AR binding units are advantageously configured to degrade not only the wild-type AR, but also one or more clinically relevant mutant forms of AR too, for example L702H. Certain PROTAC compounds of this specification also have a surprisingly beneficial combination of properties, e.g. relating to AR degradation and selectivity/safety profile in combination.

This specification relates to the above-mentioned AR-binding units and to PROTAC compounds (and pharmaceutically acceptable salts thereof) that incorporate such AR binding units together with an E3 ubiquitin ligase cereblon binder unit (the two units being linked by a linker).

This specification also relates to pharmaceutical compositions containing such PROTACs (and pharmaceutically acceptable salts thereof) and their use in methods of treatment in the human or animal body, for example in the treatment or prevention of cancer. This specification also relates to processes and intermediate compounds (and salts thereof) involved in the preparation of said PROTACs.

According to the first aspect of this specification there is provided a compound of Formula (I):

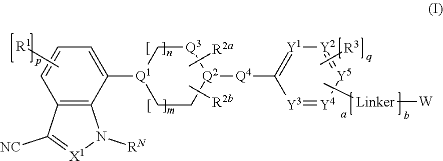

or a pharmaceutically acceptable salt thereof,
wherein:
X is CH or N;
p is 0, 1 or 2;
where:
each R¹ is a substituent on any C atom and is independently selected from F, Cl, C₁₋₃alkyl and C₁₋₃alkoxy, wherein said C₁₋₃alkyl and C₁₋₃alkoxy may be independently optionally substituted by one or more F;
R^{N} is selected from H and Me;
n is 0, 1 or 2;
m is 0 or 1;
Q¹ is CH or N;
when n & m are both other than 0, Q² is CH or N;
when n & m are both 0, Q² is CH;
when n is 0 or 1 Q³ is CH;
when n is 2 and Q² is N, Q³ is CH;
when n is 2 and Q² is CH, Q³ is CH or O;
R^{2a} and R^{2b} are substituents on the same or different C atoms other than at Q¹ or Q², each independently selected from H, F and C₁₋₃alkyl, or R^{2a} & R^{2b} together form a —(CH₂)ᵣ— group where r is 1, 2 or 3;
Q⁴ is a single bond or —NR⁴C(═O);
R⁴ is H or Me;
0, 1 or 2 of Y¹, Y², Y³, Y⁴ & Y⁵ is/are N, and are otherwise C;
each R³ is a substituent on any C atom at Y¹, Y², Y³, Y⁴ & Y⁵, and is/are independently selected from F, Cl, CN, C₁₋₃alkyl and C₁₋₃alkoxy, wherein said C₁₋₃alkyl and C₁₋₃alkoxy may be independently optionally substituted by one or more F;
q is 0, 1 or 2;
wherein Linker is attached at any available C atom at Y⁴ & Y⁵;
Linker is a saturated or a partially or fully unsaturated framework comprising C and H atoms and at least one heteroatom, wherein said framework has end points of attachment ‘a’ and ‘b’ and a minimum length of from 5 to 26 atoms between ‘a’ and ‘b’; wherein said framework may include one or more straight and/or branched chains and/or rings and is optionally substituted on any available C atom(s) by one or more F; and
W is an E3 ubiquitin ligase cereblon binder unit.

This specification also describes, in part, a pharmaceutical composition which comprises a compound of Formula (I), or a pharmaceutically acceptable salt thereof, and at least one pharmaceutically acceptable excipient.

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, a method for treating cancer in a warm-blooded animal in need of such treatment, which comprises administering to the warm-blooded animal a therapeutically effective amount of a compound of Formula (I), or a pharmaceutically acceptable salt thereof.

As shown in the experimental section hereinafter, the present researchers have not just developed a range of beneficial AR binding units, but gained an understanding about where such binding units can incorporate a linker (leading to an E3 ubiquitin ligase cereblon binder unit) without it interfering with their AR binding capability. Accordingly the present researchers understand that when incorporating their AR binding units into a PROTAC, the linker of said PROTAC should not attach at the left or central rings in Formula (I) shown hereinabove, but may suitably attach at the specified position on the right-hand ring in the compound of Formula (I) as shown herein.

Therefore, in a further aspect of this specification there is provided a PROTAC compound or a pharmaceutically acceptable salt thereof, containing an E3 ubiquitin ligase cereblon binder unit and an AR binding unit of Formula (Ia):

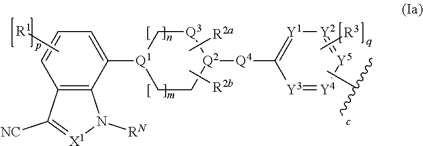

where R¹, p, X¹, R^{N}, n, m, R^{2a}, R^{2b}, Q¹, Q², Q³, Q⁴, Y¹, Y², Y³, Y⁴, Y⁵, R³ and q may take any of the values defined herein for each of these integers respectively.

For avoidance of doubt

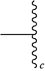

in Formula (Ia) indicates a point of connection via a single covalent bond to the remainder of the PROTAC compound.

In one embodiment there is provided a PROTAC compound or a pharmaceutically acceptable salt thereof, containing an E3 ubiquitin ligase cereblon binder unit linked to an AR binding unit of Formula (Ia) as described herein.

As described herein, the link between an E3 ubiquitin ligase cereblon binder unit and an AR binding unit of Formula (Ia) as shown hereinabove may have a length corresponding to the length of a linear chain of from 5 to 26 atoms, said atoms being linked by single covalent bonds and each selected from carbon or a heteroatom (i.e. O, N or S).

As described herein, the link between an E3 ubiquitin ligase cereblon binder unit and an AR binding unit of Formula (Ia) as shown hereinabove may have a length corresponding to the length of a linear chain of from 5 to 14 atoms, said atoms being linked by single covalent bonds and each selected from carbon or a heteroatom (i.e. O, N or S).

In one embodiment, the link between an E3 ubiquitin ligase cereblon binder unit and an AR binding unit of Formula (Ia) as shown hereinabove may have a length corresponding to the length of a linear chain of from 5 to 26 atoms, said atoms being linked by single covalent bonds and each selected from C, N or O.

In one embodiment, the link between an E3 ubiquitin ligase cereblon binder unit and an AR binding unit of Formula (Ia) as shown hereinabove may have a length corresponding to the length of a linear chain of from 5 to 14 atoms, said atoms being linked by single covalent bonds and each selected from C, N or O.

It is to be understood that although a link length “corresponding to the length of [certain atoms in a chain]” it is not to be interpreted that this limits said link to said atoms, and for example sulfur atom(s) may also be present in the link even if S atoms are not included in the length descriptor.

In a further aspect of the specification there is provided an androgen receptor binding unit of Formula (Ia), as described herein, for use in a PROTAC compound (or pharmaceutically acceptable salt thereof) that also contains an E3 ubiquitin ligase cereblon binder unit.

In a further aspect of the specification there is provided an androgen receptor binding unit of Formula (Ia), as described herein, for use in a PROTAC compound (or pharmaceutically acceptable salt thereof) that is linked to an E3 ubiquitin ligase cereblon binder unit.

Accordingly, there is provided an androgen receptor binding unit of Formula (Ia), as described herein, for use in a PROTAC compound (or pharmaceutically acceptable salt thereof) where said PROTAC compound also contains an E3 ubiquitin ligase cereblon binder unit.

Accordingly, there is provided an androgen receptor binding unit of Formula (Ia), as described herein, for use in a PROTAC compound (or pharmaceutically acceptable salt thereof) where said PROTAC compound also contains an E3 ubiquitin ligase cereblon binder unit that is linked to said androgen receptor binding unit.

Accordingly, there is provided an androgen receptor binding unit of Formula (Ia), as described herein, for incorporation into a PROTAC compound (or pharmaceutically acceptable salt thereof) where said PROTAC compound also contains an E3 ubiquitin ligase cereblon binder unit.

Accordingly, there is provided an androgen receptor binding unit of Formula (Ia), as described herein, for incorporation into a PROTAC compound (or pharmaceutically acceptable salt thereof) where said PROTAC compound also contains an E3 ubiquitin ligase cereblon binder unit that is linked to said androgen receptor binding unit.

Therefore, there is provided an androgen receptor binding unit of Formula (Ia), as described herein, contained within a PROTAC compound (or a pharmaceutically acceptable salt thereof) where said PROTAC compound also contains an E3 ubiquitin ligase cereblon binder unit.

Therefore, there is provided an androgen receptor binding unit of Formula (Ia), as described herein, contained within a PROTAC compound (or a pharmaceutically acceptable salt thereof) where said PROTAC compound also contains an E3 ubiquitin ligase cereblon binder unit that is linked to said androgen receptor binding unit.

In one embodiment the link between androgen receptor binding unit of Formula (Ia) [as described above] and an E3 ubiquitin ligase cereblon binder unit is a Linker as defined according to any embodiment or claim herein, where the point of attachment shown in Formula (Ia) above connects to the ‘a’ point of connection on any Linker as defined herein.

### DESCRIPTION OF ILLUSTRATIVE EMBODIMENTS

Many embodiments of this disclosure are detailed throughout the specification and will be apparent to a reader skilled in the art.

A pharmaceutically acceptable salt of a compound of Formula (I) or PROTAC compound described herein may be, for example, an acid-addition salt when said compound contains a basic functional group, such as an amine. An acid-addition salt may be formed using an inorganic acid or an organic acid. A pharmaceutically acceptable salt of said compound may be, for example, a base-addition salt when said compound contains an acidic functional group, such as a carboxylic acid. An acid-addition salt may be formed using an inorganic base or an organic base. “Pharmaceutically acceptable salt” is used to specify that the salt is suitable for use in the human or animal body. An example list of pharmaceutically acceptable salts can be found in the Handbook of Pharmaceutical Salts: Properties, Selection and Use, P. H. Stahl and C. G. Wermuth, editors, Weinheim/Zurich:Wiley-VCH/VHCA, 2002. A pharmaceutically acceptable salt of a compound of Formula (I) or PROTAC compound includes such salts that may be formed within the human or animal body after administration of said compound to said human or animal body.

As used herein the term “alkyl” includes straight chain, branched chain and cyclic alkyl groups and combinations thereof having the specified number of carbon atoms. Therefore, C₁₋₃alkyl includes methyl, ethyl, n-propyl, isopropyl and cyclopropyl; and C₁₋₉alkyl would include (4-isopropylcyclohexyl)methyl. The same principles apply to the term “alkoxy”. Similarly, as used herein the term “alkoxy” includes straight chain, branched chain and/or cyclic alkoxy groups having the specified number of carbon atoms. Therefore, C₁₋₃alkoxy includes methoxy, ethoxy, n-propoxy, isopropoxy and cyclopropoxy.

In this specification chemical abbreviations familiar to the skilled person may be used including for example “Me”=methyl, “Et”=ethyl, “Pr”=propyl, “Bu”=butyl and “Ph”=phenyl.

Where the term “optionally” is used, it is intended that the subsequent feature may occur or may not occur. As such, use of the term “optionally” includes instances where the feature is present, and also instances where the feature is not present. For example, “methyl optionally substituted by one or more F” includes —CH₃, —CH₂F, —CHF₂ and —CF₃.

The term “substituted” means that one or more hydrogens on the designated atom or group is replaced by the indicated substituent(s) provided that any atom(s) bearing such substituent(s) maintains its permitted valency where the skilled person understands that the standard valencies of carbon, nitrogen and oxygen are 4, 3 and 2 respectively. Therefore, “substituted on any available C atom(s)” is to be understood to mean that the substituent(s) is/are limited in their positioning (and/or potentially in their number) according to whether there are any hydrogen atoms remaining on the designated atom or group which could be replaced by said substituent(s).

The dashed bonds included in Z, [symbol] indicate the possibility that the bond may in each case be a single covalent bond or a double covalent bond—in accordance with the atom (or group of atoms) present at each of the X^{E}, X^{F}, X^{G}, X^{H} and X^{J} positions. The skilled person understands that the standard valencies of carbon, nitrogen and oxygen are as mentioned above, and as such they can understand whether each dashed bond should be interpreted as a single bond or a double bond in any given Z group in the compound of Formula (I). The same applies to the X^{E2}, X^{F2}, X^{G2}, X^{H2} and X^{J2} positions in Z^{A}.

The term “adjacent” or “adjacent position”—for example in reference to X^{G}, X^{H} and X^{J} of Z refers to the next closest position in the molecular chain/ring system. Accordingly, in the context of Z: X^{G} and X^{H} are adjacent each other, X^{H} and X^{J} are also adjacent each other, but X^{G} is not adjacent X^{J}.

The term “saturated” means that the atoms of the specified framework or group are linked only by single covalent bonds. Accordingly, the term “unsaturated” means that the specified framework or group contains double and/or triple covalent bonds. Examples of unsaturated molecular fragments that may be present within a partly or fully unsaturated group or framework are C═C, C═N, C═O, N═N, C≡C or C≡N in cases where nitrogen and oxygen heteroatoms are permitted/present, and may also include S═O in cases where sulfur heteroatoms are also permitted/present.

It is to be understood that “heteroatom” may represent an oxygen, nitrogen or sulfur atom unless explicitly further limited in a given context.

The term “minimum length of [ . . . ] atoms between ‘a’ and ‘b’” refers to the shortest chain of atoms in the chain between ‘a’ and ‘b’. Therefore, if the chain consisted of —CH₂CH₂CH₂—, the number of atoms in the chain is 3 (the hydrogen atoms are regarded as not being in the chain). Alternatively if the chain consisted of 1,3-phenylene, where the shorter route around the phenyl ring contains 3 C atoms and the long route around the phenyl ring contains 5 C atoms, the minimum length of such a chain would be counted as 3 atoms.

It is to be understood that the points of attachment ‘a’ and ‘b’ each represent single covalent bonds to the relevant adjacent groups/atoms.

It is to be understood that “direct bond” is interchangeable with “single bond”.

It is to be understood that in this specification “rings” or “heterocyclic groups” may include single rings, fused rings, spirocyclic rings and bridged rings.

In reference to the Linker, as described herein, it is to be understood that the branching, where present may be present on a chain (even a chain of 1 atom length) and/or on a ring. The skilled person would generally interpret in this manner, but for the avoidance of doubt, it is to be understood that the “branching” that occurs inherently in order to form a ring is not considered “branching” in the context of the Linker embodiments described herein. It is to be understood that said branches may occur on the same or different atoms of the Linker framework. For example it is possible to have two “═O” branches on a sulfur heteroatom in order to form a SO₂ group within the Linker framework. Linker #22 described hereinafter is an example where there is one branch (-Me) coming from a chain within the Linker.

It is further to be understood that ‘branches’ (and definitions for branches provided herein) refer to branches that branch off the main chain of atoms between ‘a’ and ‘b’, leading to a ‘dead end’ in the molecular structure.

In this specification it is to be understood that the point of attachment of a given group to some other group (via a single covalent bond) may be represented by a line meeting a bond substantially at right angles to said bond, for example as shown on the far right-hand side of Formula (Ia) herein, and for example at either end of Linkers 1 to 46 depicted hereinafter.

In this specification when “0, 1 or 2 of X^{A}, X^{B}, X^{C}, X^{D}, X^{E} & X^{F} is/are N where X^{E} & X^{F} are not both N, and are otherwise C”—certain of the C atoms are to be understood to implicitly possess a hydrogen atom where necessary in order to satisfy the standard valency (4) for carbon atoms. The skilled person will understand that such H atom cannot be present on a C at X^{E} or X^{F} or on a C at X^{A}, X^{B}, X^{C} or X^{D} when a substituent or Linker is attached to said carbon. The same principle applies in respect of X¹, X², X³, X⁴ (and with X⁵, X⁶, X⁷ & X⁸ where present), and also with Y¹, Y², Y³ & Y⁴. The same applies to X^{A2}, X^{B2}, X^{C2}, X^{D2}, X^{E2}, X^{F2}, X^{G2}, X^{H2} & X^{J2}.

In this specification a reference to a secondary or tertiary amine is intended to have the normal meaning in the art and therefore a nitrogen atom that is part of an amide group or a sulphonamide group, for example, is not to be regarded as a secondary or tertiary amine.

In this specification a saturated heterocyclic group refers to at least one ring of atoms (including bridged rings, spiro rings, fused rings, and single rings) containing carbon atoms and at least one heteroatom, where the heteroatom(s) is/are each independently selected from N, O and S, and where each atom in the ring is linked to its adjacent atoms by single covalent bonds. Therefore, an example of a heterocyclic group is a spiro heterocyclic group having two rings and a total of one heteroatom such as 9-azasprio[5.5]undecane. Typically, a saturated heterocyclic group will have at least two carbon atoms separating each of the heteroatom(s) present in said group to ensure a suitable level of chemical stability for use in a pharmaceutical context. Where reference is made to a “nitrogen-containing saturated (or partially unsaturated) heterocyclic group” this requires the presence of at least one nitrogen heteroatom but does not limit the possibility of one or more non-nitrogen heteroatoms (i.e. S, O) being present in addition. In this specification a partially unsaturated heterocyclic group refers to at least one ring of atoms (including bridged rings, spiro rings, fused rings, and single rings) containing carbon atoms and at least one heteroatom, where the heteroatom(s) is/are each independently selected from N, O and S, and where at least two atoms within the heterocyclic group are connected to each other via a double covalent bond. As the skilled person would understand, partially unsaturated does not include fully unsaturated heterocyclic groups—i.e. where the group contains the maximum possible number of double bonds for the atomic framework in question.

Where reference is made to a cyclic group (e.g. a heterocyclic group) having a specified number of ring atoms, this includes the atoms making up the ring (including atoms involved in the bridge of a bridged ring, and all atoms of a fused or spiro ring) but does not include any hydrogen atoms or other substituent atoms attached to the ring atoms. Therefore, for example, a cyclic group which is 1,4-piperazin-1,4-diyl has 6 ring atoms (4C and 2N).

In this specification an alkylene group (for example a C₁₋₅alkylene) is a saturated group consisting only of carbon and hydrogen atoms with two points of attachment to adjacent atoms/groups. They may include straight chain(s), branched chain(s) and/or ring(s). Accordingly C₁alkylene represents —CH₂—, a C₂alkylene can represent —CH₂CH₂— or —CH(Me)-, C₁₋₅alkylene includes for example —CH₂(cyclobut-1,3-diyl)-. A “straight chain Cᵤ₁₋ᵤ₂alkylene” corresponds to —(CH₂)ᵤ— where u is an integer from u1 to u2.

In this specification a hydrocarbyl group means any group consisting only of C and H atoms. For example, C₁₋₇hydrocarbyl includes methyl, phenyl and p-tolyl.

The term “therapy” is intended to have its normal meaning of dealing with a disease in order to entirely or partially relieve one, some or all of its symptoms, or to correct or compensate for the underlying pathology. The terms “therapeutic” and “therapeutically” should be interpreted in a corresponding manner.

The term “treatment” is used synonymously with “therapy”. Similarly the term “treat” can be regarded as “applying therapy” where “therapy” is as defined herein.

Some values of variable groups are as follows. One, two or more of such values, may be used in any combination with any other definitions, claims, aspects or embodiments herein (unless the context doesn't permit) to provide further embodiments/claims of the specification.

In one embodiment X¹ is CH.

In one embodiment X¹ is N.

In one embodiment R^{N} is H.

In one embodiment R^{N} is Me.

In one embodiment X¹ is CH and R^{N} is H.

In one embodiment X¹ is CH and R^{N} is Me.

In one embodiment X¹ is N and R^{N} is H.

In one embodiment X¹ is N and R^{N} is Me.

In one embodiment p is 0.

In one embodiment p is 0 or 1.

In one embodiment p is 1 or 2.

In one embodiment p is 1.

In one embodiment p is 2.

When p is 1, R¹ may be bound para to Q¹.

When p is 2, one R¹ may be bound para to Q¹, and the other R¹ may be ortho or meta to Q¹.

In one embodiment each R¹ is selected from F, Cl, methyl, CF₃, methoxy and OCF₃.

In one embodiment each R¹ is selected from F, Cl and methyl.

In one embodiment p is 1 and R¹ is selected from F, Cl and methyl.

In one embodiment p is 1 and R¹ is selected from F and Cl.

In one embodiment n is 0.

In one embodiment n is 1.

In one embodiment n is 2.

In one embodiment m is 0.

In one embodiment m is 1.

In one embodiment n is 0 and m is 1.

In one embodiment n is 1 and m is 1.

In one embodiment n is 2 and m is 0.

In one embodiment n is 0, m is 1, Q¹ is N and Q² is CH.

In one embodiment n is 1, m is 1, Q¹ is CH and Q² is CH.

In one embodiment n is 1, m is 1, Q¹ is N and Q² is CH or N.

In one embodiment n is 2, m is 0, Q¹ is N and Q² is CH.

In one embodiment n is 2, m is 0, Q¹ is N, Q² is CH and Q³ is CH.

In one embodiment n is 2, m is 0, Q¹ is N, Q² is CH and Q³ is O.

In one embodiment Q¹ is N, Q² is CH and Q³ is CH.

In one embodiment Q¹ is N, Q² is CH and Q³ is CH, and n and m are 1 and 1 or 2 and 0 respectively.

In one embodiment R^{2a} is H and R^{2b} is H.

In one embodiment n is 1, m is 0, R^{2a} is H and R^{2b} is C₁₋₃alkyl attached to a C atom other than at Q¹ and Q², where said C atom has an (R)-stereochemical configuration.

In one embodiment n is 1, m is 0, R^{2a} is H and R^{2b} is C₁₋₃alkyl attached to a C atom other than at Q¹ and Q², where said C atom has an (S)-stereochemical configuration.

In one embodiment n is 1, m is 1, R^{2a} is H and R^{2b} is C₁₋₃alkyl attached to a C atom other than at Q¹ and Q², where said C atom has an (R)-stereochemical configuration.

In one embodiment n is 1, m is 1, R^{2a} is H and R^{2b} is C₁₋₃alkyl attached to a C atom other than at Q¹ and Q², where said C atom has an (S)-stereochemical configuration.

In one embodiment Q¹ is CH with an (R)-stereochemical configuration at said C atom.

In one embodiment Q¹ is CH with an (S)-stereochemical configuration at said C atom.

In one embodiment n is 2; m is 0; R^{2a} & R^{2b} are both H; Q¹ is CH and Q² is N.

In one embodiment n is 2; m is 0; R^{2a} & R^{2b} are both H; Q² is N and Q¹ is CH with an (R)-stereochemical configuration at said C atom.

In one embodiment n is 2; m is 0; R^{2a} & R^{2b} are both H; Q² is N and Q¹ is CH with an (S)-stereochemical configuration at said C atom.

In one embodiment Q² is CH with an (R)-stereochemical configuration at said C atom.

In one embodiment Q² is CH with an (S)-stereochemical configuration at said C atom.

In one embodiment n is 2; m is 0; R^{2a} & R^{2b} are both H; Q¹ is N and Q² is CH.

In one embodiment n is 2; m is 0; R^{2a} & R^{2b} are both H; Q¹ is N and Q² is CH with an (R)-stereochemical configuration at said C atom.

In one embodiment n is 2; m is 0; R^{2a} & R^{2b} are both H; Q¹ is N and Q² is CH with an (S)-stereochemical configuration at said C atom.

In one embodiment R^{2a} and R^{2b} are substituents on the same or different C atoms other than at Q, each independently selected from H and C₁₋₃alkyl.

In one embodiment R^{2a} and R^{2b} are substituents on the same or different C atoms other than at Q, each independently selected from H and Me.

In one embodiment R^{2a} and R^{2b} are substituents on the same or different C atoms adjacent Q and are otherwise as defined herein.

In one embodiment R^{2a} and R^{2b} are substituents on the same or different C atoms adjacent Q, each independently selected from H and C₁₋₃alkyl (for example Me).

In one embodiment R^{2a} and R^{2b} are substituents on the same or different C atoms and are both H.

In one embodiment R^{2a} and R^{2b} are substituents on the same or different C atoms other than at Q, each independently selected from H, F and C₁₋₃alkyl.

In one embodiment R^{2a} and R^{2b} are substituents on the same or different C atoms other than at Q, each independently selected from H and C₁₋₃alkyl, or R^{2a} & R^{2b} together form a —(CH₂)ᵣ— group where r is 1, 2 or 3.

In one embodiment R^{2a} and R^{2b} are substituents on the same or different C atoms other than at Q, where R^{2a} is Me and R^{2b} is H.

In one embodiment R^{2a} and R^{2b} are substituents on the same C atom other than at Q, where R^{2a} and R^{2b} are both Me.

In one embodiment R^{2a} and R^{2b} are substituents on the same C atom adjacent Q, where R^{2a} and R^{2b} are both Me.

In one embodiment Q⁴ is a single bond.

In one embodiment Q⁴ is a NR⁴C(═O) and R⁴ is H.

In one embodiment Q⁴ is a NR⁴C(═O) and R⁴ is methyl.

In one embodiment Q⁴ is a NR⁴C(═O), R⁴ is H and Q² is CH.

In one embodiment Q⁴ is a NR⁴C(═O), R⁴ is methyl and Q² is CH.

In one embodiment Y¹, Y², Y³, Y⁴ & Y⁵ are respectively selected from (C, C, C, C, C), (N, C, C, C, C), (C, N, C, C, C), (N, C, C, N, C), (N, N, C, C, C), (N, C, N, C, C), (C, N, C, N, C) and (C, N, N, C, C).

In one embodiment Y¹, Y², Y³, Y⁴ & Y⁵ are respectively selected from (C, C, C, C, C), (N, C, C, C, C) and (N, N, C, C, C).

In one embodiment 0 or 1 of Y¹, Y², Y³, Y⁴ & Y⁵ is N and are otherwise C.

In one embodiment 1 or 2 of Y, Y², Y³, Y⁴ & Y⁵ is/are N and are otherwise C.

In one embodiment 1 of Y¹, Y², Y³, Y⁴ & Y⁵ is N and are otherwise C.

In one embodiment Y¹ is N; and Y², Y³, Y⁴ & Y⁵ are all C.

In one embodiment Y² is N; and Y², Y³, Y⁴ & Y⁵ are all C.

In one embodiment 2 of Y², Y³, Y⁴ & Y⁵ are N and are otherwise C.

In one embodiment Y¹ & Y³ are N; and Y², Y⁴ and Y⁵ are C.

In one embodiment Y¹ & Y² are N; and Y³, Y⁴ and Y⁵ are C.

In one embodiment Y¹ & Y⁴ are N; and Y², Y³ and Y⁵ are C.

In one embodiment Y² & Y³ are N; and Y, Y⁴ and Y⁵ are C.

In one embodiment Y² & Y⁴ are N; and Y, Y³ and Y⁵ are C.

In one embodiment Y¹, Y², Y³, Y⁴ & Y⁵ are all C.

In one embodiment q is 0 or 1.

In one embodiment q is 0.

In one embodiment q is 1.

In one embodiment q is 2.

In one embodiment q is 0, 1 or 2 and R³ (when present) is a substituent on any C atom at Y¹, Y², Y³, Y⁴ & Y⁵ selected from F, CN and C₁₋₃alkyl.

In one embodiment q is 0, 1 or 2 and R³ (when present) is a substituent on any C atom at Y¹, Y², Y³, Y⁴ & Y⁵ selected from F, CN and methyl.

In one embodiment q is 0 or 1 and R³ (when present) is a substituent on any C atom at Y¹, Y², Y³, Y⁴ & Y⁵ which is F.

In one embodiment R³ (when present) is C₁₋₃alkyl.

In one embodiment R³ (when present) is Me.

In one embodiment R³ (when present) is F.

In one embodiment R³ (when present) is CN.

In one embodiment q is 1 and R³ is attached to C at Y¹.

In one embodiment q is 1 and R³ is attached to C at Y¹, and R³ is selected from F, CN and Me.

In one embodiment q is 1 and R³ is attached to C at Y², and R³ is selected from CN and Me.

In one embodiment q is 2 and both R³ groups are F or one group is F and the other group is Me.

In one embodiment q is 2 and the R³ groups are attached to C at Y¹ and Y³.

In one embodiment q is 2 and each R³ group is F, which are attached to C at Y¹ and Y³.

In one embodiment q is 2, one R³ group is F, which is attached to C at Y¹ and one R³ group is Me, which is attached to C at Y³.

In one embodiment, Linker is attached at Y⁵.

In one embodiment, Linker is attached at Y⁴.

In one embodiment, the group of Formula (Ia) is of Formula (Ia-1):

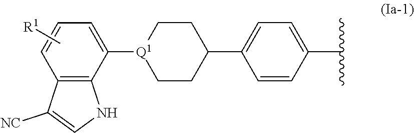

where R¹ and Q¹ are as defined above. Q¹ is CH or N.

In one embodiment, the group of Formula (Ta) is of Formula (Ia-2):

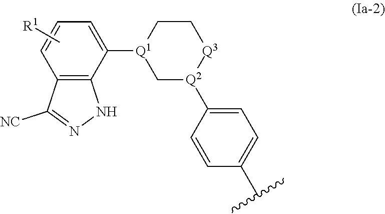

where R¹, Q¹, Q² and Q³ are as defined above. Q¹ is CH or N; Q² is CH or N; when Q² is N, Q³ is CH; when Q² is CH, Q³ is CH or O.

In one embodiment, the group of Formula (Ia) is of Formula (Ia-3):

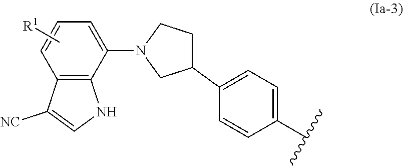

where R¹ is as defined above.

In one embodiment Linker is a saturated or a partially or fully unsaturated framework comprising C and H atoms and at least one heteroatom, wherein said framework has end points of attachment ‘a’ and ‘b’ and a minimum length of from 5 to 26 atoms between ‘a’ and ‘b’; wherein said framework may include one or more straight and/or branched chains and/or rings and is optionally substituted on any available C atom(s) by one or more F.

In one embodiment the framework of the Linker is a saturated or partially unsaturated framework.

In one embodiment the framework of the Linker is a saturated framework.

In one embodiment the framework of the Linker comprises C and H atoms and at least two heteroatoms.

In one embodiment the framework of the Linker comprises C and H atoms and at least two heteroatoms selected from O and N.

In one embodiment the framework of the Linker comprises C and H atoms and at least one nitrogen heteroatom.

In one embodiment the framework of the Linker comprises C and H atoms and at least two heteroatoms including at least one nitrogen heteroatom.

In one embodiment the framework of the Linker comprises C and H atoms and at least one nitrogen heteroatom in the form of a secondary or tertiary amine.

In one embodiment the framework of the Linker comprises C and H atoms and at least one nitrogen heteroatom in the form of a tertiary amine.

In one embodiment the framework of the Linker comprises C and H atoms and at least two heteroatoms including at least one nitrogen heteroatom in the form of a secondary or tertiary amine.

In one embodiment the framework of the Linker comprises C and H atoms and at least two heteroatoms including at least one nitrogen heteroatom in the form of a tertiary amine.

In one embodiment the Linker has a minimum length of from 5 to 20 atoms between ‘a’ and ‘b’.

In one embodiment the Linker has a minimum length of from 5 to 15 atoms between ‘a’ and ‘b’.

In one embodiment the Linker has a minimum length of from 5 to 14 atoms between ‘a’ and ‘b’.

In one embodiment the total number of C and hetero atoms in the Linker framework is from 6 to 26.

In one embodiment the total number of C and hetero atoms in the Linker framework is from 7 to 24.

In one embodiment the total number of C and hetero atoms in the Linker framework is from 7 to 22.

In one embodiment the total number of C and hetero atoms in the Linker framework is from 7 to 20.

In one embodiment the total number of C and hetero atoms in the Linker framework is from 7 to 18.

In one embodiment when W is —Z—R^{A}, the Linker is attached at any available C atom at X^{B} or X^{C} of Z.

In one embodiment the framework of the Linker may include one or more straight chains and/or rings and is optionally substituted on any available C atom(s) by one or more F.

In one embodiment the framework of the Linker consists of one or more straight chains and/or rings that are optionally substituted on any available C atom(s) by one or more F.

In one embodiment the framework of the Linker may include (or consist of) one or more straight and/or branched chains and/or rings (wherein the total number of branches is from 0 to 5) that are optionally substituted on any available C atom(s) by one or more F.

In one embodiment the framework of the Linker may include (or consist of) one or more straight and/or branched chains and/or rings (wherein the total number of branches is from 0 to 3) that are optionally substituted on any available C atom(s) by one or more F.

In one embodiment the framework of the Linker may include (or consist of) one or more straight and/or branched chains and/or rings (wherein the total number of branches is 0 or 1) that are optionally substituted on any available C atom(s) by one or more F.

In one embodiment the total number of branches is 0.

In one embodiment the total number of branches is 1.

In one embodiment the total number of branches is 2.

In one embodiment the total number of branches is 3.

In one embodiment any/each branch in the framework of a Linker has from 1 to 5 C and/or hetero atoms.

In one embodiment any/each branch in the framework of a Linker has 1 or 2 C and/or hetero atoms.

In one embodiment any/each branch in the framework of a Linker has 1 C and/or hetero atom.

In one embodiment any/each branch in the framework of a Linker has 1 C atom.

In one embodiment the total number of C and/or heteroatoms in the branch(es) (where present) of the framework of the Linker is from 1 to 5.

In one embodiment the total number of C and/or heteroatoms in the branch(es) (where present) of the framework of the Linker is from 1 to 3.

In one embodiment the total number of C and/or heteroatoms in the branch(es) (where present) of the framework of the Linker is 1.

In one embodiment the framework of the Linker is either unbranched or has one branch that is Me.

In one embodiment the framework of the Linker is unbranched.

In one embodiment the framework of the Linker is optionally substituted on any available C atom(s) by 1 or 2 F (for example by 2 F, for example where said 2 F are substituted on the same carbon atom).

In one embodiment the framework of the Linker is not substituted by any F.

In one embodiment the Linker is a saturated or a partially unsaturated framework comprising C and H atoms and at least one heteroatom, wherein said framework has end points of attachment ‘a’ and ‘b’ and: a minimum length of from 5 to 14 atoms between ‘a’ and ‘b’; wherein the total number of C and hetero atoms in the Linker framework is from 7 to 18; where said framework comprises one or more straight and/or rings that are optionally substituted on any available C atom(s) by 1 or 2 F (for example by 2 F).

In one embodiment the framework of the Linker includes at least one nitrogen-containing saturated or partially unsaturated heterocyclic group (e.g. having from 4 to 12 ring atoms) or a A¹-CH₂—CH₂-A² unit where Ar¹ and A² are each independently selected from N and O.

In one embodiment the framework of the Linker includes at least one nitrogen-containing saturated heterocyclic group (e.g. having from 4 to 12 ring atoms) or a Ar—CH₂—CH₂-A² unit where A¹ and A² are each independently selected from N and O.

In one embodiment the framework of the Linker includes a A1-CH₂—CH₂-A² unit where A¹ and A² are each independently selected from N and O.

In one embodiment the framework of the Linker includes at least one nitrogen-containing saturated or partially unsaturated heterocyclic group (e.g. having from 4 to 12 ring atoms) or a A¹-CH₂—CH₂-A² unit where one of A¹ & A² is N and the other of A¹ & A² is selected from N or O.

In one embodiment the framework of the Linker includes at least one nitrogen-containing saturated heterocyclic group (e.g. having from 4 to 12 ring atoms) or a A¹-CH₂—CH₂-A² unit where one of A¹ & A² is N and the other of A¹ & A² is selected from N or O.

In one embodiment the framework of the Linker includes a A¹-CH₂—CH₂-A² unit where one of A¹ & A² is N and the other of A¹ & A² is selected from N or O.

In one embodiment the framework of the Linker includes at least one nitrogen-containing saturated or partially unsaturated heterocyclic group (e.g. having from 4 to 12 ring atoms) or a A¹-CH₂—CH₂-A² unit where one of A¹ & A² is N in the form of a secondary or tertiary amine, and the other of A¹ & A² is selected from N or O.

In one embodiment the framework of the Linker includes a A1-CH₂—CH₂-A² unit where one of A¹ & A² is N in the form of a secondary or tertiary amine, and the other of A¹ & A² is selected from N or O.

In one embodiment the framework of the Linker includes at least one saturated or partially unsaturated heterocyclic group.

In one embodiment the framework of the Linker includes at least one saturated heterocyclic group.

In one embodiment the framework of the Linker includes at least one nitrogen-containing saturated or partially unsaturated heterocyclic group.

In one embodiment the framework of the Linker includes at least one nitrogen-containing saturated heterocyclic group.

In one embodiment the framework of the Linker includes at least one nitrogen-containing saturated or partially unsaturated heterocyclic group having from 4 to 12 ring atoms.

In one embodiment the framework of the Linker includes at least one nitrogen-containing saturated heterocyclic group having from 4 to 12 ring atoms.

In one embodiment the framework of the Linker includes:
(1) a A1-CH₂—CH₂-A² unit where A1 and A² are each independently selected from N and O; and/or
(2) at least one nitrogen-containing saturated or partially unsaturated heterocyclic group selected from piperazine, azetidine, piperidine, 1,4-diazepane, 12-oxa-3,9-diazaspiro[5.6]dodecane, pyrrolidine, 3,9-diazaspiro-[5.5]undecane, 2,5-diazabicyclo[2.2.1]heptane, 1,2,3,3a,4,5,6,6a-octahydropyrrolo[3,4-c]pyrrole, 1,2,3,6-tetrahydropyridine, morpholine, 2-azaspiro[3.5]nonane and 9-azaspiro[5.5]undecane.

In one embodiment the framework of the Linker includes:
(1) a A1-CH₂—CH₂-A² unit where one of A¹ & A² is N in the form of a secondary or tertiary amine, and the other of A¹ & A² is selected from N or O; and/or
(2) at least one nitrogen-containing saturated or partially unsaturated heterocyclic group selected from piperazine, azetidine, piperidine, 1,4-diazepane, 12-oxa-3,9-diazaspiro[5.6]dodecane, pyrrolidine, 3,9-diazaspiro-[5.5]undecane, 2,5-diazabicyclo[2.2.1]heptane, 1,2,3,3a,4,5,6,6a-octahydropyrrolo[3,4-c]pyrrole, 1,2,3,6-tetrahydropyridine, morpholine, 2-azaspiro[3.5]nonane and 9-azaspiro[5.5]undecane.

In one embodiment the framework of the Linker includes at least one nitrogen-containing saturated or partially unsaturated heterocyclic group selected from piperazine, azetidine, piperidine, 1,4-diazepane, 12-oxa-3,9-diazaspiro[5.6]dodecane, pyrrolidine, 3,9-diazaspiro[5.5]undecane, 2,5-diazabicyclo[2.2.1]heptane, 1,2,3,3a,4,5,6,6a-octahydropyrrolo[3,4-c]pyrrole, 1,2,3,6-tetrahydropyridine, morpholine, 2-azaspiro[3.5]nonane and 9-azaspiro[5.5]undecane.

In one embodiment the framework of the Linker includes an O—CH₂—CH₂—N unit.

In one embodiment the framework of the Linker includes an O—CH₂CH₂—O—CH₂CH₂—N unit.

In one embodiment the framework of the Linker includes a piperazine group.

In one embodiment the framework of the Linker includes an azetidine group.

In one embodiment the framework of the Linker includes a piperidine group.

In one embodiment the framework of the Linker includes a 1,4-diazepane group.

In one embodiment the framework of the Linker includes a 12-oxa-3,9-diazaspiro[5.6]dodecane group.

In one embodiment the framework of the Linker includes a pyrrolidine group.

In one embodiment the framework of the Linker includes a 3,9-diazaspiro[5.5]undecane group.

In one embodiment the framework of the Linker includes a 2,5-diazabicyclo[2.2.1]heptane group.

In one embodiment the framework of the Linker includes a 1,2,3,3a,4,5,6,6a-octahydropyrrolo[3,4-c]pyrrole group.

In one embodiment the framework of the Linker includes a 1,2,3,6-tetrahydropyridine group.

In one embodiment the framework of the Linker includes a morpholine group.

In one embodiment the framework of the Linker includes a 2-azaspiro[3.5]nonane group.

In one embodiment the framework of the Linker includes a 9-azaspiro[5.5]undecane group.

In one embodiment the framework of the Linker includes at least two nitrogen-containing saturated or partially unsaturated heterocyclic groups.

In one embodiment the framework of the Linker includes at least two nitrogen-containing saturated heterocyclic groups.

In one embodiment the framework of the Linker includes a C, N or O atom at the ‘a’ point of attachment.

In one embodiment the framework of the Linker includes an N or O atom at the ‘a’ point of attachment.

In one embodiment the framework of the Linker includes an N or O atom at the ‘b’ point of attachment.

In one embodiment the framework of the Linker includes a C atom at the ‘a’ point of attachment.

In one embodiment the framework of the Linker includes an N atom at the ‘a’ point of attachment.

In one embodiment the framework of the Linker includes a C or N atom at the ‘b’ point of attachment.

In one embodiment the framework of the Linker includes an N atom at the ‘b’ point of attachment.

In one embodiment the framework of the Linker includes a C atom at the ‘b’ point of attachment.

In one embodiment the framework of the Linker includes an O atom at the ‘a’ point of attachment.

In one embodiment the framework of the Linker includes an O atom at the ‘b’ point of attachment.

In one embodiment the framework of the Linker includes an N or O atom at both the ‘a’ and ‘b’ points of attachment.

In one embodiment the framework of the Linker includes a C, N or O atom at the ‘a’ point of attachment and a C or N atom at the ‘b’ point of attachment.

In one embodiment the framework of the Linker includes:
(1) a A1-CH₂—CH₂-A² unit where one of A¹ & A² is N in the form of a secondary or tertiary amine, and the other of A¹ & A² is selected from N or O; and/or
(2) at least one nitrogen-containing saturated or partially saturated heterocyclic group selected from piperazin-1,4-diyl, azetidin-1,3-diyl, piperidin-1,4-diyl, 1,4-diazepan-1,4-diyl, 12-oxa-3,9-diazaspiro[5.6]dodecan-3,9-diyl, pyrrolidin-1,3-diyl, 3,9-diazaspiro[5.5]undecan-3,9-diyl, 2,5-diazabicyclo[2.2.1]heptan-2,5-diyl, 1,3,3a,4,6,6a-hexahydropyrrolo[3,4-c]pyrrol-2,5-diyl, 3,6-dihydro-2H-pyridin-1,4-diyl, morpholin-2,3-diyl, 2-azaspiro[3.5]nonan-2,7-diyl and 9-azaspiro[5.5]undecan-3,9-diyl.

In one embodiment the framework of the Linker includes at least one nitrogen-containing saturated or partially saturated heterocyclic group selected from piperazin-1,4-diyl, azetidin-1,3-diyl, piperidin-1,4-diyl, 1,4-diazepan-1,4-diyl, 12-oxa-3,9-diazaspiro[5.6]dodecan-3,9-diyl, pyrrolidin-1,3-diyl, 3,9-diazaspiro[5.5]undecan-3,9-diyl, 2,5-diazabicyclo[2.2.1]heptan-2,5-diyl, 1,3,3a,4,6,6a-hexahydropyrrolo[3,4-c]pyrrol-2,5-diyl, 3,6-dihydro-2H-pyridin-1,4-diyl, morpholin-2,3-diyl, 2-azaspiro[3.5]nonan-2,7-diyl and 9-azaspiro[5.5]undecan-3,9-diyl.

In one embodiment the framework of the Linker includes a piperazin-1,4-diyl group.

In one embodiment the framework of the Linker includes an azetidin-1,3-diyl group.

In one embodiment the framework of the Linker includes a piperidin-1,4-diyl group.

In one embodiment the framework of the Linker includes a 1,4-diazepan-1,4-diyl group.

In one embodiment the framework of the Linker includes a 12-oxa-3,9-diazaspiro[5.6]dodecan-3,9-diyl group.

In one embodiment the framework of the Linker includes a pyrrolidin-1,3-diyl group.

In one embodiment the framework of the Linker includes a 3,9-diazaspiro[5.5]undecan-3,9-diyl group.

In one embodiment the framework of the Linker includes a 2,5-diazabicyclo[2.2.1]heptan-2,5-diyl group.

In one embodiment the framework of the Linker includes a 1,3,3a,4,6,6a-hexahydropyrrolo[3,4-c]pyrrol-2,5-diyl group.

In one embodiment the framework of the Linker includes a 3,6-dihydro-2H-pyridin-1,4-diyl group.

In one embodiment the framework of the Linker includes a morpholin-2,3-diyl group.

In one embodiment the framework of the Linker includes a 2-azaspiro[3.5]nonan-2,7-diyl group.

In one embodiment the framework of the Linker includes a 9-azaspiro[5.5]undecan-3,9-diyl group.

In one embodiment the Linker has the Formula:

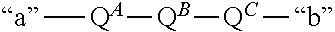

wherein:
‘a’ and ‘b’ represent the end points of attachment;
Q^{A} is -G-Q^{H}- or -G-(C₁₋₅alkylene)-;
Q^{B} is a direct bond, -Q^{B1}-Q^{B2}-Q^{B3}- or C₁₋₃alkylene optionally substituted by one or more F (for example 1 or 2 F, for example 2 F); where:
   Q^{B1} & Q^{B3} each independently represent a direct bond or C₁₋₂alkylene;
   Q^{B2} is Q^{H}, —O—CH₂CH₂—O—, —O— or —N(R^{J})— where R¹ is H or C₁₋₃alkyl;
Q^{C} is -Q^{H}-G- or —(C₁₋₅alkylene)-G-;
each G is independently a direct bond, —CH₂—, —O—, —C(═O)— or —N(R^{G})— where each R^{G} is independently H or C₁₋₃alkyl;
each Q^{H} is independently a 4-12-membered nitrogen-containing saturated or partially unsaturated heterocyclic group;
where the values of Q^{A}, Q^{B} and Q^{C} are selected so that the Linker does not contain any N—N or N—O bonds.

In one embodiment the Linker has the Formula:

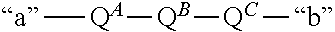

wherein:
‘a’ and ‘b’ represent the end points of attachment;
Q^{A} is -G-Q^{H}- or -G-(C₁₋₅alkylene)-;
Q^{B} is a direct bond, -Q^{B1}-Q^{B2}-Q^{B3}- or C₁₋₃alkylene optionally substituted by one or more F (e.g. 1 or 2);
where:
   Q^{B1} & Q^{B3} each independently represent a direct bond or C₁₋₂alkylene;
   Q^{B2} is Q^{H}, —O—CH₂CH₂—O— or —N(R^{J})— where R^{J} is C₁₋₃alkyl;
Q^{C} is -Q^{H}-G- or —(C₁₋₅alkylene)-G-;
each G is independently a direct bond, —CH₂—, —C(═O)—, —O—, or —N(R^{G})— where each R^{G} is independently H or C₁₋₃alkyl;
each Q^{H} is independently a 4-12-membered nitrogen-containing saturated or partially unsaturated heterocyclic group;
where the values of Q^{A}, Q^{B} and QC are selected so that the Linker does not contain any N—N or N—O bonds.

In one embodiment the Linker has the Formula:

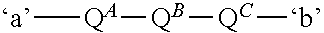

wherein:
‘a’ and ‘b’ represent the end points of attachment;
Q^{A} is -G^{A}-Q^{HA}- or -G^{A}-(C₁₋₅alkylene)-; where G^{A} is a direct bond, —CH₂—, —C(═O)—, —O—, —NH— or —N(Me)-;
   Q^{HA} is a 4 to 11-membered nitrogen-containing saturated heterocyclic group;
Q^{B} is a direct bond, -Q^{Bf}-Q^{B2}-Q^{B3}- or C₁₋₃alkylene optionally substituted by one or more F (e.g. 1 or 2);
where:
   Q^{B1} & Q^{B3} each independently represent a direct bond or C₁₋₂alkylene;
   Q^{B2} is piperazin-1,4-diyl, —O—CH₂CH₂—O— or —N(R^{J})— where R^{J} is C₁₋₃alkyl;
Q^{C} is -Q^{HC}-G^{C}- or —(C₁₋₂alkylene)-G^{C}-; where G^{C} is a direct bond, —O— or —NH—;
   Q^{HC} is a 6 to 12-membered nitrogen-containing saturated or partially unsaturated heterocyclic group;
where the values of Q^{A}, Q^{B} and Q^{C} are selected so that the Linker does not contain any N—N or N—O bonds.

In one embodiment Q^{A} is -G^{A}-Q^{HA}- or -G^{A}-(C₁₋₅alkylene)- where:
G^{A} is selected from a direct bond, —CH₂—, —C(═O)—, —O— or —N(R^{G})—.
each R^{G} is independently H or C₁₋₃alkyl; and
Q^{HA} is a 4 to 11-membered nitrogen-containing saturated heterocyclic group.

In one embodiment Q^{A} is -G^{A}-Q^{HA}- or -G^{A}-(C₁₋₅alkylene)- where:
G^{A} is selected from a direct bond, —CH₂—, —C(═O)—, —O— or —N(R^{G})—.
each R^{G} is independently H or C₁₋₃alkyl; and
Q^{HA} is selected from azetidin-1,3-diyl, morpholin-2,3-diyl, pyrrolidin-1,3-diyl, piperidin-1,4-diyl, piperazin-1,4-diyl, 3,9-diazaspiro[5.5]undecan-3,9-diyl, 2-azaspiro[3.5]nonan-2,7-diyl and 9-azaspiro[5.5]undecan-3,9-diyl.

In one embodiment Q^{A} is -G^{A}-Q^{HA}- or -G^{A}-(C₁₋₅alkylene)- where:
G^{A} is selected from a direct bond, —CH₂—, —C(═O)—, —O— or —NH— or —N(Me)-;
Q^{HA} is selected from azetidin-1,3-diyl, pyrrolidin-1,3-diyl, piperidin-1,4-diyl, piperazin-1,4-diyl, 3,9-diazaspiro[5.5]undecan-3,9-diyl and 9-azaspiro[5.5]undecan-3,9-diyl; and the C₁₋₅alkylene within said -G^{A}-(C₁₋₅alkylene)- is selected from —(CH₂)_{f}— where f is an integer from 1 to 5, cyclobut-1,3-diyl and —CH₂(cyclobut-1,3-diyl)-.

In one embodiment Q^{A} is selected from azetidin-1,3-diyl, pyrrolidin-1,3-diyl, piperidin-1,4-diyl, —O(piperidin-1,4-diyl)-, —C(O)-piperidin-1,4-diyl, —CH₂(piperidin-1,4-diyl)-, piperazin-1,4-diyl, 3,9-diazaspiro[5.5]undecan-3,9-diyl, morpholin-2,3-diyl, 2-azaspiro[3.5]nonan-2,7-diyl, —O(9-azaspiro[5.5]undecan-3,9-diyl)-, —O(cyclobut-1,3-diyl)-, —OCH₂(cyclobut-1,3-diyl)-, —(CH₂)_{f}—, —O—(CH₂)_{f}—, —NH—(CH₂)_{f}— and —N(Me)-(CH₂)_{f}—, where f is an integer from 1 to 5.

In one embodiment Q^{A} is azetidin-1,3-diyl.

In one embodiment Q^{A} is pyrrolidin-1,3-diyl.

In one embodiment Q^{A} is piperidin-1,4-diyl.

In one embodiment Q^{A} is —C(O)-piperidin-1,4-diyl.

In one embodiment Q^{A} is —O(piperidin-1,4-diyl)-.

In one embodiment Q^{A} is —CH₂(piperidin-1,4-diyl)-.

In one embodiment Q^{A} is piperazin-1,4-diyl.

In one embodiment Q^{A} is —CH₂(piperazin-1,4-diyl)-.

In one embodiment Q^{A} is morpholin-2,3-diyl.

In one embodiment Q^{A} is 2-azaspiro[3.5]nonan-2,7-diyl.

In one embodiment Q^{A} is 3,9-diazaspiro[5.5]undecan-3,9-diyl.

In one embodiment Q^{A} is -0(9-azaspiro[5.5]undecan-3,9-diyl)-.

In one embodiment Q^{A} is -G-(C₁₋₅alkylene)-.

In one embodiment Q^{A} is selected from —O(cyclobut-1,3-diyl)-, —OCH₂(cyclobut-1,3-diyl)-, —(CH₂)_{f}—, —O(CH₂)_{f}—, —NH—(CH₂)_{f}— and —N(Me)-(CH₂)_{f}—, where f is an integer from 1 to 5.

In one embodiment Q^{A} is —O(cyclobut-1,3-diyl)-.

In one embodiment Q^{A} is —OCH₂(cyclobut-1,3-diyl)-.

In one embodiment Q^{A} is —(CH₂)_{f}— where f is an integer from 1 to 4 (for example, f is 4) In one embodiment Q^{A} is —O—(CH₂)_{f}— where f is an integer from 1 to 5 (for example, f is 4).

In one embodiment Q^{A} is —NH—(CH₂)_{f}— where f is an integer from 1 to 5.

In one embodiment Q^{A} is —N(Me)-(CH₂)_{f}—, where f is an integer from 1 to 4.

In one embodiment Q^{B} is a direct bond, -Q^{B1}-Q^{B2}-Q^{B3}- or a straight chain C₁₋₃alkylene optionally substituted by one or more F (for example 1 or 2 F, for example by 2 F).

In one embodiment Q^{B} is a direct bond.

In one embodiment Q^{B} is -Q^{B1}-Q^{B2}-Q^{B3}-.

In one embodiment Q^{B} is C₁₋₃alkylene optionally substituted by one or more F.

In one embodiment Q^{B} is C₁₋₃alkylene optionally substituted by one or two F (for example by 2 F).

In one embodiment Q^{B} is a straight chain C₁₋₃alkylene optionally substituted by 1 or 2 F (for example by 2 F).

In one embodiment Q^{B} is C₁₋₃alkylene.

In one embodiment Q^{B} is a straight chain C₁₋₃alkylene.

In one embodiment Q^{B} is —CF₂—CH₂—CH₂— or —(CH₂)_{w}— where w is 1 to 3.

In one embodiment Q^{B1} and Q^{B3} each independently represent a direct bond, —CH₂— or —CH₂CH₂—.

In one embodiment Q^{B}1 is a direct bond or —CH₂—.

In one embodiment Q^{B}1 is a direct bond.

In one embodiment Q^{B1} is —CH₂—.

In one embodiment Q^{B3} is a direct bond, —CH₂— or —CH₂CH₂—.

In one embodiment Q^{B3} is a direct bond.

In one embodiment Q^{B3} is —CH₂—.

In one embodiment Q^{B3} is —CH₂CH₂—.

In one embodiment Q^{B2} is Q^{H}, —O—CH₂CH₂—O— or —N(R^{J})— where R^{J} is C₁₋₃alkyl.

In one embodiment Q^{B2} is Q^{H}, —O—CH₂CH₂—O— or —N(Me)-.

In one embodiment Q^{B2} is piperazin-1,4-diyl, azetidin-1,3-diyl, —O—CH₂CH₂—O— or —N(Me)-.

In one embodiment Q^{B2} is piperazin-1,4-diyl, —O—CH₂CH₂—O— or —N(Me)-.

In one embodiment Q^{B2} is Q^{H}.

In one embodiment Q^{B2} is piperazin-1,4-diyl.

In one embodiment Q^{B2} is azetidin-1,3-diyl.

In one embodiment Q^{B2} is —O—CH₂CH₂—O—.

In one embodiment Q^{B2} is —N(R^{J})— where R^{J} is C₁₋₃alkyl.

In one embodiment Q^{B}2 is —N(Me)-.

In one embodiment QC is -Q^{H}-G- or —(C₁₋₄alkylene)-G-.

In one embodiment QC is -Q^{H}-G- or —(C₁₋₃alkylene)-G-.

In one embodiment QC is -Q^{H}-G- or —(C₁₋₂alkylene)-G-.

In one embodiment QC is -Q^{HC}-G^{C}- or —(C₁₋₅alkylene)-G^{C}- where:
G^{C} is selected from a direct bond, —O— or —NH—; and
Q^{HC} is a 6 to 12-membered nitrogen-containing saturated or partially unsaturated heterocyclic group.

In one embodiment Q^{C} is -QHC-G^{C}- or —(C₁₋₂alkylene)-G^{C}- where:
G^{C} is selected from a direct bond, —O— or —NH—; and
Q^{HC} is a 6 to 12-membered nitrogen-containing saturated or partially unsaturated heterocyclic group.

In one embodiment Q^{C} is -Q^{HC}-G^{C}- or —(C₁₋₂alkylene)-G^{C}- where:
G^{C} is selected from a direct bond, —O— or —NH—; and
Q^{HC} is selected from piperazin-1,4-diyl, piperidin-1,4-diyl, 2,5-diazabicyclo[2.2.1]heptan-2,5-diyl, 1,4-diazepan-1,4-diyl, 3,9-diazaspiro[5.5]undecan-3,9-diyl, 12-oxa-3,9-diazaspiro[5.6]-dodecan-3,9-diyl, 1,3,3a,4,6,6a-hexahydropyrrolo[3,4-c]pyrrol-2,5-diyl and 3,6-dihydro-2H-pyridin-1,4-diyl.

In one embodiment Q^{C} is selected from piperazin-1,4-diyl, piperidin-1,4-diyl, -(piperidin-1,4-diyl)O—, 2,5-diazabicyclo[2.2.1]heptan-2,5-diyl, 1,4-diazepan-1,4-diyl, 3,9-diazaspiro[5.5]undecan-3,9-diyl, 12-oxa-3,9-diazaspiro[5.6]-dodecan-3,9-diyl, 1,3,3a,4,6,6a-hexahydropyrrolo[3,4-c]pyrrol-2,5-diyl, 3,6-dihydro-2H-pyridin-1,4-diyl, and —CH₂CH₂—NH— and —(CH₂)_{g}—O— where g is an integer from 1 to 4.

In one embodiment Q^{C} is piperazin-1,4-diyl.

In one embodiment Q^{C} is piperidin-1,4-diyl.

In one embodiment Q^{C} is -(piperidin-1,4-diyl)O—.

In one embodiment Q^{C} is 2,5-diazabicyclo[2.2.1]heptan-2,5-diyl.

In one embodiment Q^{C} is 1,4-diazepan-1,4-diyl.

In one embodiment Q^{C} is 3,9-diazaspiro[5.5]undecan-3,9-diyl.

In one embodiment Q^{C} is 12-oxa-3,9-diazaspiro[5.6]dodecan-3,9-diyl.

In one embodiment Q^{C} is 1,3,3a,4,6,6a-hexahydropyrrolo[3,4-c]pyrrol-2,5-diyl.

In one embodiment Q^{C} is 3,6-dihydro-2H-pyridin-1,4-diyl.

In one embodiment Q^{C} is —(C₁₋₅alkylene)-G- [for example, —(CH₂)_{g}—O— where g is an integer from 1 to 5].

In one embodiment Q^{C} is —(C₁₋₄alkylene)-G- [for example, —(CH₂)_{g}—O— where g is an integer from 1 to 4].

In one embodiment Q^{C} is —(C₁₋₃alkylene)-G- [for example, —(CH₂)_{g}—O— where g is 1, 2 or 3].

In one embodiment Q^{C} is —(C₁₋₂alkylene)-G- [for example, —(CH₂)_{g}—O— where g is 1 or 2].

In one embodiment Q^{C} is —(CH₂)_{g}-G^{C}- where g is an integer from 1 to 5 and G^{C} is —O— or —NH—.

In one embodiment Q^{C} is —(CH₂)_{g}-G^{C}- where g is 1 or 2 and G^{C} is —O— or —NH—.

In one embodiment Q^{C} is —CH₂CH₂NH—.

In one embodiment Q^{C} is —CH₂—.

In one embodiment each Q^{H} is independently selected from piperazin-1,4-diyl, azetidin-1,3-diyl, piperidin-1,4-diyl, 1,4-diazepan-1,4-diyl, 12-oxa-3,9-diazaspiro[5.6]dodecan-3,9-diyl, pyrrolidin-1,3-diyl, 3,9-diazaspiro[5.5]undecan-3,9-diyl, 2,5-diazabicyclo[2.2.1]heptan-2,5-diyl, 1,3,3a,4,6,6a-hexahydropyrrolo[3,4-c]pyrrol-2,5-diyl, 3,6-dihydro-2H-pyridin-1,4-diyl and 9-azaspiro[5.5]undecan-3,9-diyl.

In one embodiment each Q^{H} is independently selected from piperazin-1,4-diyl, azetidin-1,3-diyl, piperidin-1,4-diyl, 1,4-diazepan-1,4-diyl, 12-oxa-3,9-diazaspiro[5.6]dodecan-3,9-diyl, pyrrolidin-1,3-diyl, 3,9-diazaspiro[5.5]undecan-3,9-diyl, morpholin-2,3-diyl, 2-azaspiro[3.5]nonan-2,7-diyl, 2,5-diazabicyclo[2.2.1]heptan-2,5-diyl, 1,3,3a,4,6,6a-hexahydropyrrolo[3,4-c]pyrrol-2,5-diyl and 9-azaspiro[5.5]undecan-3,9-diyl.

In one embodiment Q^{H} is piperazin-1,4-diyl.

In one embodiment Q^{H} is azetidin-1,3-diyl.

In one embodiment Q^{H} is piperidin-1,4-diyl.

In one embodiment Q^{H} is 1,4-diazepan-1,4-diyl.

In one embodiment Q^{H} is 12-oxa-3,9-diazaspiro[5.6]dodecan-3,9-diyl.

In one embodiment Q^{H} is pyrrolidin-1,3-diyl.

In one embodiment Q^{H} is 3,9-diazaspiro[5.5]undecan-3,9-diyl.

In one embodiment Q^{H} is 2,5-diazabicyclo[2.2.1]heptan-2,5-diyl.

In one embodiment Q^{H} is 1,3,3a,4,6,6a-hexahydropyrrolo[3,4-c]pyrrol-2,5-diyl.

In one embodiment Q^{H} is 3,6-dihydro-2H-pyridin-1,4-diyl.

In one embodiment Q^{H} is 9-azaspiro[5.5]undecan-3,9-diyl.

In one embodiment Q^{H} is 2-azaspiro[3.5]nonan-2,7-diyl.

In one embodiment Q^{H} is morpholin-2,3-diyl.

In one embodiment R^{J} is C₁₋₃alkyl.

In one embodiment R^{J} is Me.

In one embodiment R^{G} is C₁₋₃alkyl.

In one embodiment R^{G} is H or Me.

In one embodiment R^{G} is Me.

In one embodiment R^{G} is H.

In one embodiment the Linker (for example ‘a’ -Q^{A}-Q^{B}-Q^{C}- ‘b’) is selected from any of Linkers 1 to 58 shown below:

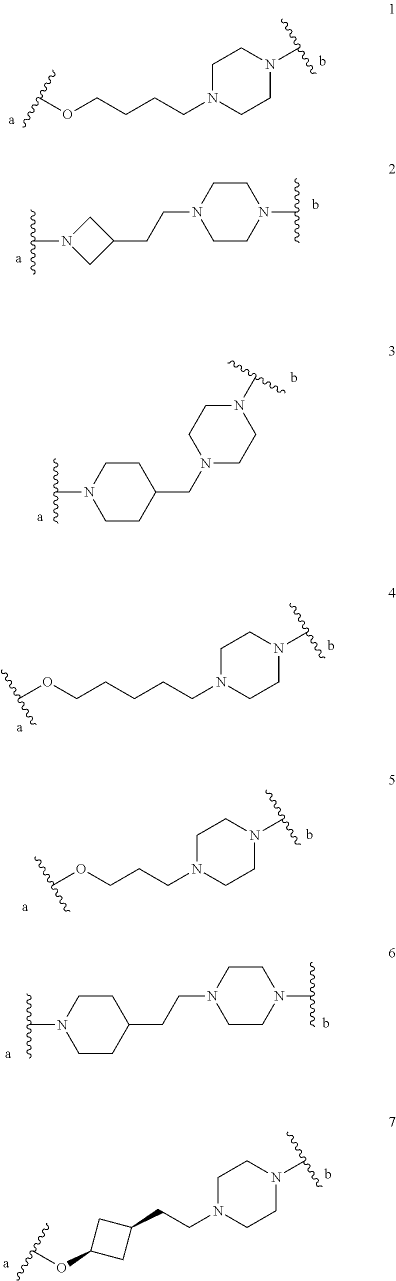

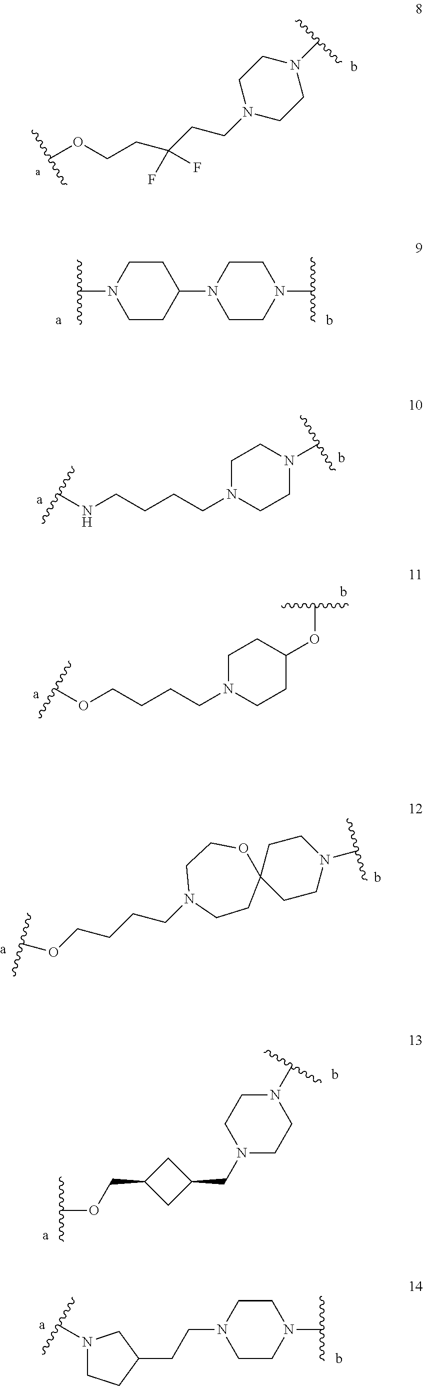

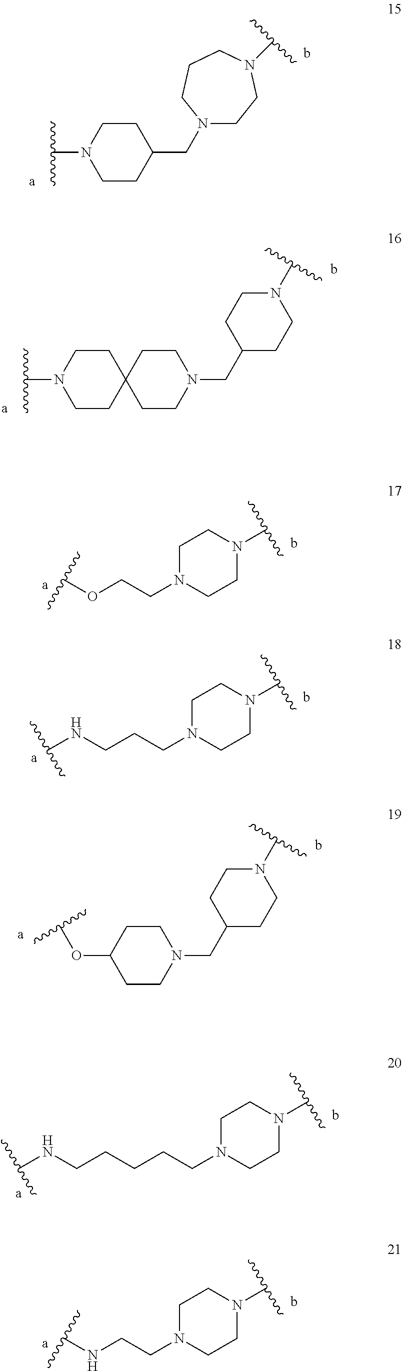

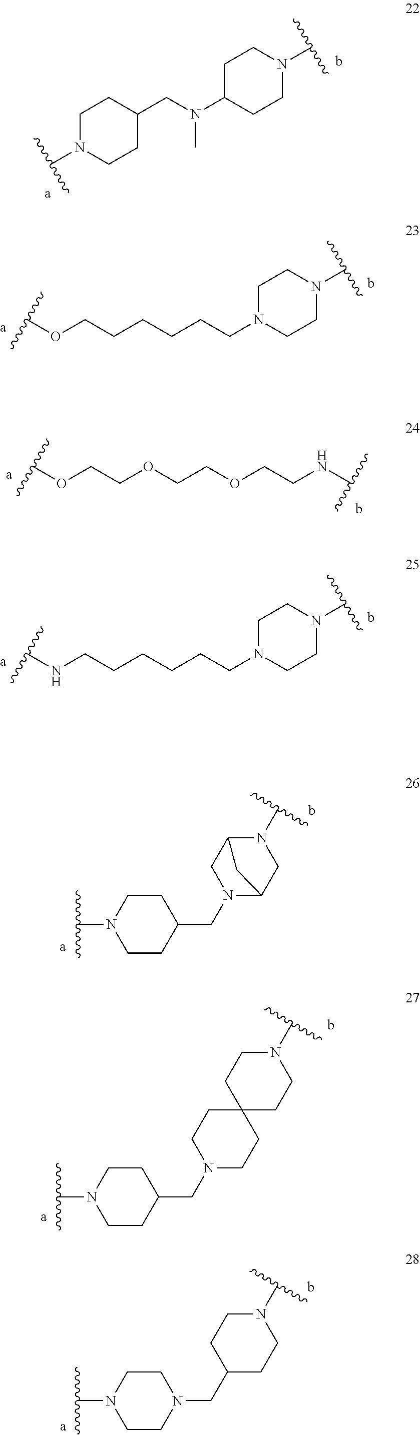

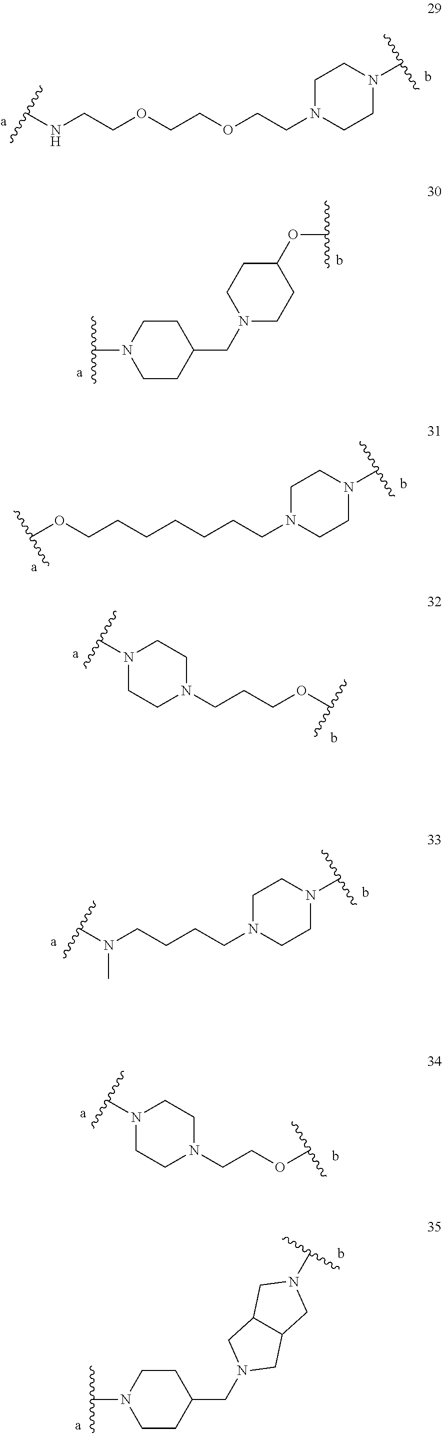

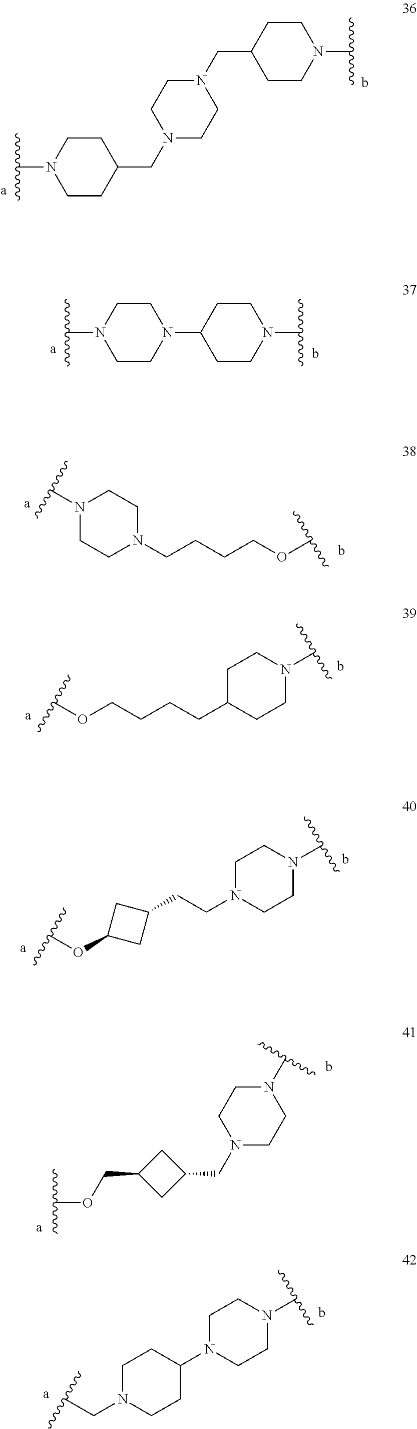

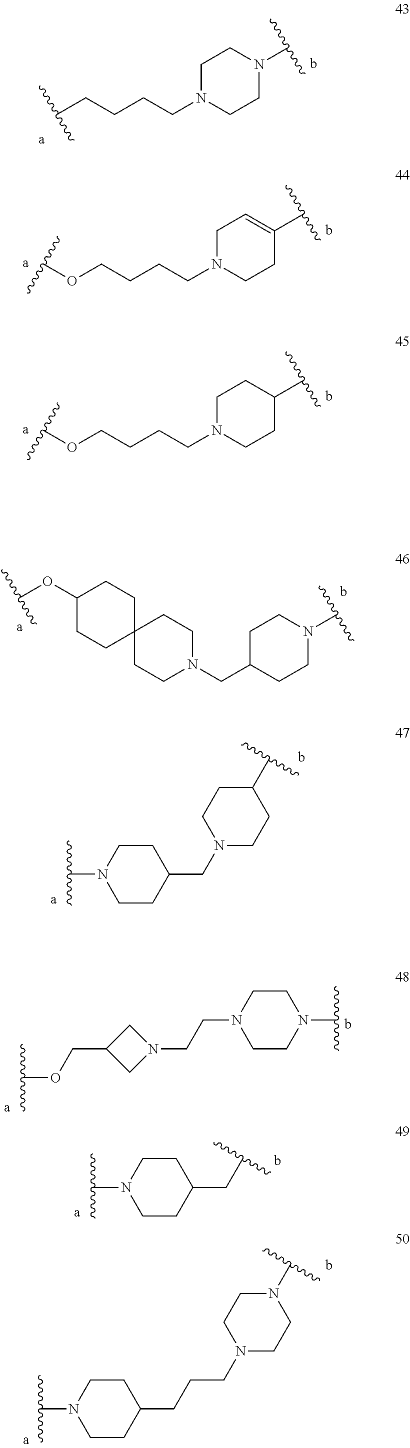

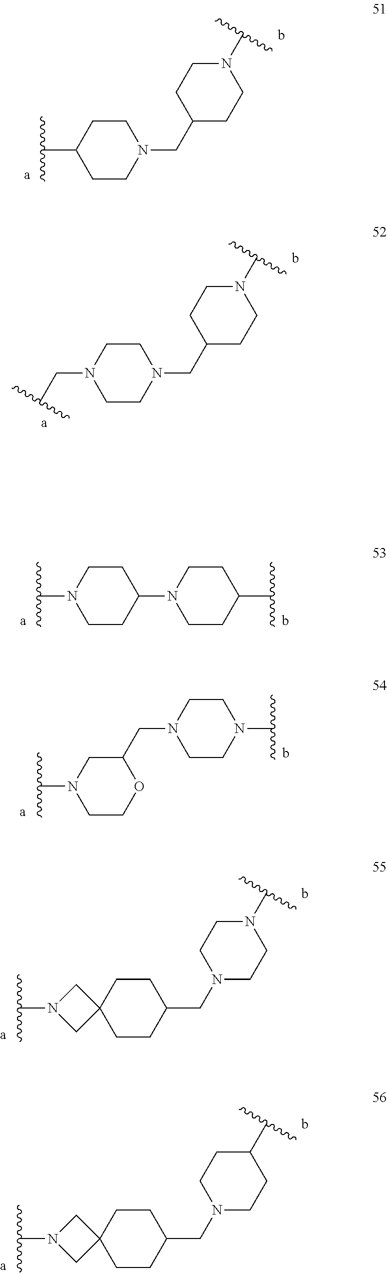 [SMILES: *C1CCN(CC2CCN(*)CC2)CC1 ; *CN1CCN(CC2CCN(*)CC2)CC1 ; *C1CCN(C2CCN(*)CC2)CC1 ; *N1CCN(CC2CN(*)CCO2)CC1 ; *N1CCN(CC2CCC3(CC2)CN(*)C3)CC1 ; *C1CCN(CC2CCC3(CC2)CN(*)C3)CC1]

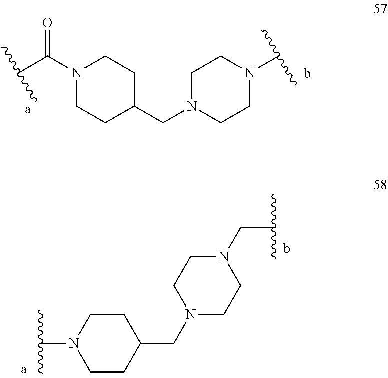 [SMILES: *C(=O)N1CCC(CN2CCN(*)CC2)CC1 ; *CN1CCN(CC2CCN(*)CC2)CC1]

In one embodiment the Linker (for example ‘a’-Q^{A}-Q^{B}-Q^{C}- ‘b’) is selected from any of Linkers 1, 3, 6, 9, 19, 28, 45, 47, 49, 50, 51, 52, 53, 54, 55, 56, 57 and 58.

In some embodiments Linker 54 is 54a:

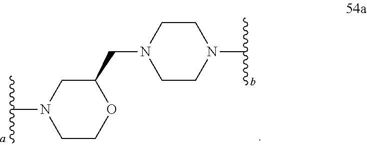 [SMILES: *N1CCN(C[C@@H]2CN(*)CCO2)CC1]

In one embodiment W is an E3 ubiquitin ligase cereblon binder unit attached to the ‘b’ end of the Linker via an available C atom within said E3 ubiquitin ligase cereblon binder unit.

In one embodiment W is W1 which is: —Z—(R^{A})ₕ
where Z is:

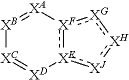

wherein
[symbol] represents a single covalent bond or a double covalent bond;
0, 1 or 2 of X^{A}, X^{B}, X^{C}, X^{D}, X^{E} & X^{F} is/are N, where X^{E} & X^{F} are not both N, and are otherwise C;
1 of X^{G}, X^{H} & X^{J} is C(O);
1 of X^{G}, X^{H} & X^{J} is N-(2,6-dioxopiperidin-3-yl) (Y); and
of X^{G}, X^{H} & X^{J} is selected from C(R^{T})₂, —CH₂CH₂—, C(O), N(C₁₋₃alkyl), —O— and —N═, wherein each R is selected from H, F, Me or together with the carbon of C(RT)₂ forms a cycloprop-1,1-diyl group; where X^{G}, X^{H} & X^{J} are selected such that there are not two C(O) groups present at adjacent positions, and that the N-(2,6-dioxopiperidin-3-yl) is not at an adjacent position to either N(C₁₋₃alkyl) or 0;
each R^{A} is independently a substituent on any available C atom at X^{A}, X^{B}, X^{C} or X^{D} selected from F, Cl, C₁₋₃alkyl, C₁₋₃alkoxy wherein said C₁₋₃alkyl and C₁₋₃alkoxy is independently optionally substituted by one or more F;
h is 0, 1 or 2; and
wherein the Linker is attached to any C atom at X^{A}, X^{B}, X^{C} or X^{D}.

Y is:

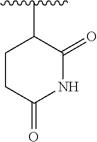 [SMILES: *C1CCC(=O)NC1=O]

In one embodiment 0 or 1 of X^{A}, X^{B}, X^{C}, X^{D}, X^{E} & X^{F} is N and are otherwise C.

In one embodiment 1 or 2 of X^{A}, X^{B}, X^{C}, X^{D}, X^{E} & X^{F} is/are N and are otherwise C.

In one embodiment X^{A}, X^{B}, X^{C}, X^{D}, X^{E} & X^{F} are all C.

In one embodiment 1 of X^{A}, X^{B}, X^{C}, X^{D}, X^{E} & X^{F} is N and are otherwise C.

In one embodiment 2 of X^{A}, X^{B}, X^{C}, X^{D}, X^{E} & X^{F} are N where X^{E} & X^{F} are not both N, and are otherwise C.

In one embodiment X^{B}, X^{D} & X^{F} are all C, and 0, 1 or 2 of X^{A}, X^{C} & X^{E} is/are N, and are otherwise C.

In one embodiment X^{B}, X^{D} & X^{F} are all C, and 0, 1 or 2 of X^{A}, X^{C} & X^{E} is/are N, and are otherwise C; where the Linker is attached to X^{B} or to X^{A} when X^{A} is C.

In one embodiment X^{B}, X^{D} & X^{F} are all C, and 0, 1 or 2 of X^{A}, X^{C} & X^{E} is/are N, and are otherwise C; where the Linker is attached to X^{B}.

In one embodiment X^{G}—X^{H}—X^{J} is:
(i) X^{G}—NY—C(O) where X^{G} is —CH₂—, —CH₂CH₂—, ═N— or C(O);
(ii) X^{G}—C(O)—NY where X^{G} is —O— or N(C₁₋₃alkyl) [for example N(Me)]; or
(iii) C(O)—NY—CH₂.

In one embodiment the Linker is attached to a C atom at X^{A} or X^{B} and X^{G}—X^{H}—X^{J} is:
(i) X^{G}—NY—C(O) where X^{G} is —CH₂—, —CH₂CH₂—, ═N— or C(O);
(ii) X^{G}—C(O)—NY where X^{G} is —O— or N(C₁₋₃alkyl) [for example N(Me)]; or
(iii) C(O)—NY—CH₂.

In one embodiment the Linker is attached to a C atom at X^{B} and X^{G}—X^{H}—X^{J} is:
(i) X^{G}—NY—C(O) where X^{G} is —CH₂—, —CH₂CH₂—, ═N— or C(O);
(ii) X^{G}—C(O)—NY where X^{G} is —O— or N(C₁₋₃alkyl) [for example N(Me)]; or
(iii) C(O)—NY—CH₂.

In one embodiment X^{G}—X^{H}—X^{J} is CH₂—NY—C(O).

In one embodiment X^{G}—X^{H}—X^{J} is CH₂CH₂—NY—C(O).

In one embodiment X^{G}—X¹—X^{J} is ═N—NY—C(O).

In one embodiment X^{G}—X¹—X^{J} is C(O)—NY—C(O).

In one embodiment X^{G}—X¹—X^{J} is O—C(O)—NY.

In one embodiment X^{G}—X¹—X^{J} is N(C₁₋₃alkyl)-C(O)—NY [for example N(Me)-C(O)—NY].

In one embodiment X^{G}—X^{H}—X^{J} is C(O)—NY—CH₂.

In one embodiment each R^{A} is a substituent on any available C at X^{A}, X^{B}, X^{C} or X^{D} selected from F, Cl and C₁₋₃alkoxy optionally substituted by one or more F.

In one embodiment each R^{A} is a substituent on any available C at X^{A}, X^{B}, X^{C} or X^{D} selected from F, Cl, OMe and —OCHF₂.

In one embodiment h is 0.

In one embodiment h is 1.

In one embodiment h is 2.

In one embodiment the Linker is attached to X^{C}, where X^{C} is C; and X^{A} is C attached to R^{A} where R^{A} is OMe where said methyl is optionally substituted by one or more F (e.g. —OCHF₂).

In one embodiment the Linker is attached to X^{C}, where X^{C} is C; and X^{A} is C attached to R^{A} where R^{A} is OMe or —OCHF₂.

In one embodiment —Z—(R^{A})ₕ together represent any one of groups 1, 4, 16, 17 and 19 (referring to the specific —Z—(R^{A})ₕ groups 1 to 21 whose structures are drawn out below).

In one embodiment h is 1 and R^{A} is C₁₋₃alkoxy [for example OMe].

In one embodiment h is 1 or 2, one R^{A} is OMe and the other R^{A} (when present, i.e. when h is 2) is Cl.

In one embodiment h is 1 or 2, one R^{A} is Cl and the other R^{A} (when present, i.e. when h is 2) is OMe.

In one embodiment h is 1 and R^{A} is F.

In one embodiment h is 1 and R^{A} is Cl.

In one embodiment h is 1 and R^{A} is —OCHF₂.

In one embodiment the Linker is attached to a C at X^{A} or X^{B}, and h is 0.

In one embodiment the Linker is attached to a C at X^{A} or X^{B}, h is 1 and R^{A} is a substituent on X^{C} where X^{C} is C and R^{A} is F.

In one embodiment the Linker is attached to a C at X^{A} or X^{B}, h is 1 and R^{A} is a substituent on X^{D} where X^{D} is C and R^{A} is OMe or —OCHF₂.

In one embodiment the Linker is attached to a C at X^{B}, h is 0, 1 or 2; the first R^{A} (when present, i.e. when h is 1 or 2) is a substituent on an available C at X^{D} and the second R^{A} (when present, i.e. when h is 2) is on an available C at X^{A}.

In one embodiment the Linker is attached to a C at X^{B}, h is 0, 1 or 2; the first R^{A} (when present, i.e. when h is 1 or 2) is a substituent on an available C at X^{D} selected from Cl, OMe and —OCHF₂; and the second R^{A}(when present, i.e. when h is 2) is on an available C at X^{A} and is selected from Cl and OMe.

In one embodiment —Z—(R^{A})ₕ together represent any of the groups 1 to 21, 27 and 32 shown below:

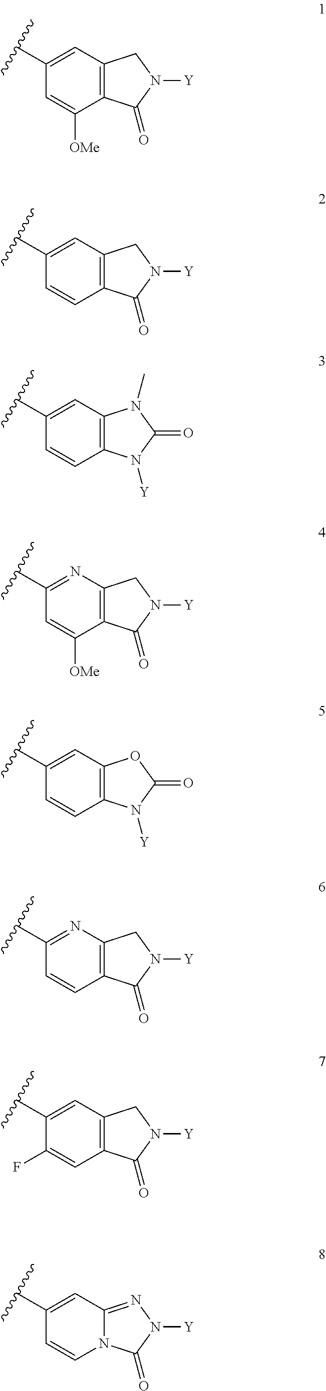

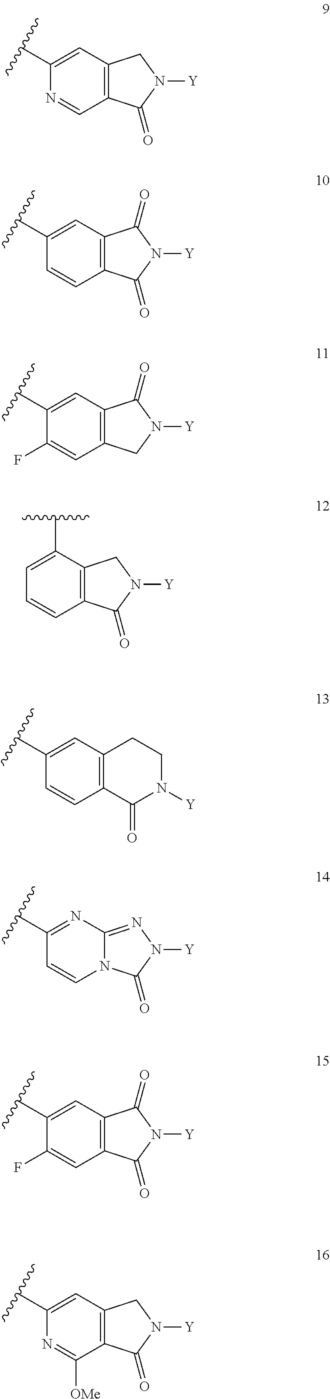

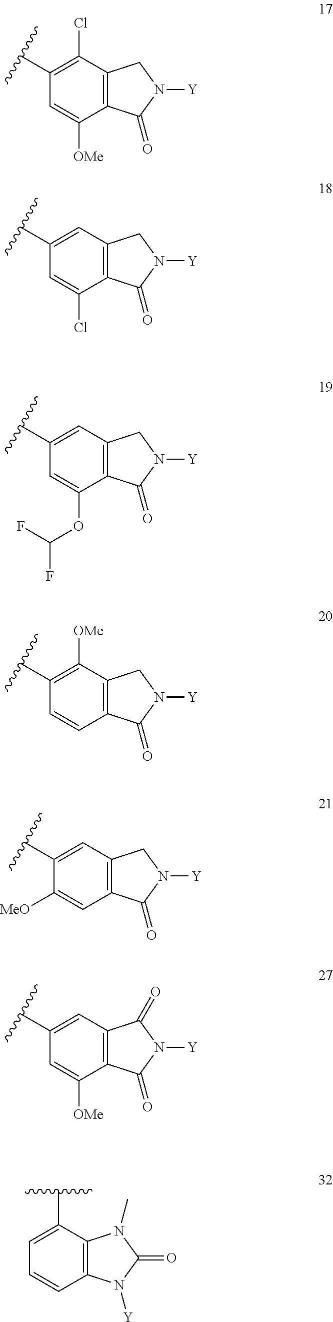

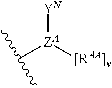

In one embodiment W is W2 which is
Z^{A} is:

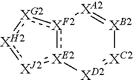

wherein
[symbol] represents a single covalent bond or a double covalent bond;
1 of X^{A2}, X^{B2}, X^{C2} & X^{D2} is C and covalently bound to Y^{N}.
0, 1 or 2 of X^{A2}, X^{B2}, X^{C2}, X^{D2}, X^{E2} & X^{F2} is/are N (where X^{E2} & X^{F2} are not both N) and are otherwise C;
1 or 2 of X^{G2}, X^{H2} & X^{J2} is/are N; and are otherwise C;
each R^{AA} is a substituent on any available C or N atom of Z—in each case independently selected from R^{AA1} optionally substituted by one or more R^{AA2}; where R^{AA} is further selected from R^{AA2} when R^{AA} is a substituent on an available C atom of Z^{A};
each R^{AA1} is independently C₁₋₄alkyl, C₂₋₃alkenyl, C₂₋₃alkynyl, C₁₋₃alkoxyC₁₋₃alkyl, carboxyC₁₋₃alkyl, C₅₋₇carbocyclyl or a 4-6 membered heterocyclyl;
each R^{AA2} is independently selected from F, Cl, Br, CN, NH₂, C₁₋₃alkyl, O(C₁₋₃alkyl), NH(C₁₋₃alkyl) and N(C₁₋₃alkyl)₂; wherein said C₁₋₃alkyls are optionally substituted by one or more F;
v is 0, 1 or 2;
Y^{N} is 2,4-dioxohexahydropyrimidin-1-yl.
Y^{N} is:

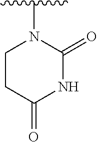 [SMILES: *N1CCC(=O)NC1=O]

In one embodiment Z^{A} is:

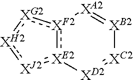

wherein
[symbol] represents a single covalent bond or a double covalent bond;
1 of X^{A2} & X^{B2} is C and covalently bound to Y^{N} and the other of X^{A2} & X^{B2} is C;
0 or 1 of X^{C2} & X^{D2} is N and is/are otherwise C;
1 of X^{G2} & X^{J2} is N and the other of X^{G2}& X^{J2} is C; and
X^{H2}, X^{E2} & X^{F2} are all C.

In one embodiment —Z^{A}—Y^{N} together represent any one or more of the groups A1 to A5 shown below, where in each case said Z^{A} group is optionally substituted on available C and/or N atom(s) by —[R^{AA}]v as further defined herein.

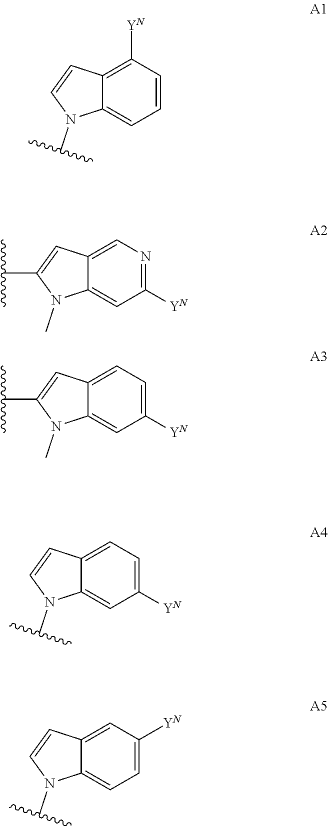

In one embodiment each R^{AA} is a substituent on any available C or N atom of Z^{A}—in each case independently selected from R^{AA1} optionally substituted by one or more R^{AA2}; where R^{AA} is further selected from R^{AA2} when R^{AA} is a substituent on an available C atom of Z; wherein each R^{AA1} is independently C₁₋₄alkyl or a 4-6 membered heterocyclyl; and each R^{AA2} is independently selected from F, Cl, CN and C₁₋₃ alkyl.

In one embodiment each R^{AA} is a substituent on any available C or N atom of Z^{A}—in each case independently selected from R^{AA1} optionally substituted by one or more R^{AA2}; where R^{AA} is further selected from R^{AA2} when R^{AA} is a substituent on an available C atom of Z^{A}; wherein each R^{AA1} is independently methyl, isopropyl, cyclopropyl, pyridinyl or pyrazolyl; and each R^{AA2} is independently F, Cl, CN or methyl.

In one embodiment each R^{AA} is a substituent on an available C atom of Z^{A}, each independently selected from methyl, isopropyl, cyclopropyl, pyridin-2-yl, 1-methylpyrazol-4-yl, —CH₂CN, F, Cl, CN; and/or a methyl substituent on an available N atom of Z^{A}.

In one embodiment v is 0.

In one embodiment v is 1.

In one embodiment v is 2.

In one embodiment v is 1 or 2.

In one embodiment v is 0 or 1.

In one embodiment v is 1; X^{A1} is a C atom; and R^{AA} is a substituent on X^{A1}.

In one embodiment v is 1; X^{A1} is a C atom; and R^{AA} is a C₁₋₄alkyl substituent on X^{A1}.

In one embodiment v is 1; X^{B1} is a C atom; and R^{AA} is a substituent on X^{B1}.

In one embodiment v is 1; X^{B1} is a C atom; and R^{AA} is a C₁₋₄alkyl (e.g. methyl) substituent on X^{B1}.

In one embodiment v is 1; X^{C1} is a C atom; and R^{AA} is a substituent on X^{C1} In one embodiment v is 1; X^{C1} is a C atom; and R^{AA} is a C₁₋₄alkyl (e.g. methyl) substituent on X^{C1}.

In one embodiment v is 1; X^{D1} is a C atom; and R^{AA} is a substituent on X^{D1}.

In one embodiment v is 1; X^{D1} is a C atom; and R^{AA} is a C₁₋₄alkyl (e.g. methyl) or F substituent on X^{D1}.

In one embodiment v is 1; X^{G1} is a C atom; and R^{AA} is a substituent on X^{G1}.

In one embodiment v is 1; X^{G1} is a C atom; and R^{AA} is a substituent on X^{G1}; where R^{AA} is selected from R^{AA1} optionally substituted by one or more R^{AA2}; or R^{AA} is selected from R^{AA2}; wherein R^{AA1} is C¹⁻⁴alkyl or a 4-6 membered heterocyclyl; and R^{AA2} is selected from F, Cl, CN and C₁₋₃alkyl.

In one embodiment v is 1; X^{G1} is a C atom; and R^{AA} is a substituent on X^{G1}; where R^{AA} is selected from methyl, isopropyl, cyclopropyl, pyridine-2-yl, 1-methylpyrazol-4-yl, —CH₂CN, F, Cl and CN.

In one embodiment v is 1 or 2; X^{G1} is a C atom; and one/the R^{AA} is a substituent on X^{G1}; where R^{AA} is selected from R^{AA} optionally substituted by one or more R^{AA2}; or R^{AA} is selected from R^{AA2}; wherein R^{AA1} is C₁₋₄alkyl or a 4-6 membered heterocyclyl; and R^{AA2} is selected from F, Cl, CN and C₁₋₃alkyl; and when v is 2, the additional R^{AA} is a fluoro substituted on an available C atom of Z^{A}.

In one embodiment v is 1; X^{G1} is a N atom; and R^{AA} is a substituent on X^{G1}.

In one embodiment v is 1; X^{G1} is a N atom; and R^{AA} is a C₁₋₄alkyl (e.g. methyl) substituent on X^{G1}.

In one embodiment v is 1; X^{H1} is a C atom; and R^{AA} is a substituent on X^{H1}.

In one embodiment v is 1; X^{H1} is a C atom; and R^{AA} is a substituent on X^{H1} that is CN or C₁₋₄alkyl (e.g. methyl).

In one embodiment v is 1 or 2; X¹ and X^{G1} are both C atoms, one or both of which are substituted by R^{AA} where each R^{A} is independently selected from CN or C₁₋₄alkyl (e.g. methyl).

In one embodiment the group:

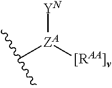

together represents one or more of the groups A1 to A9, shown below:

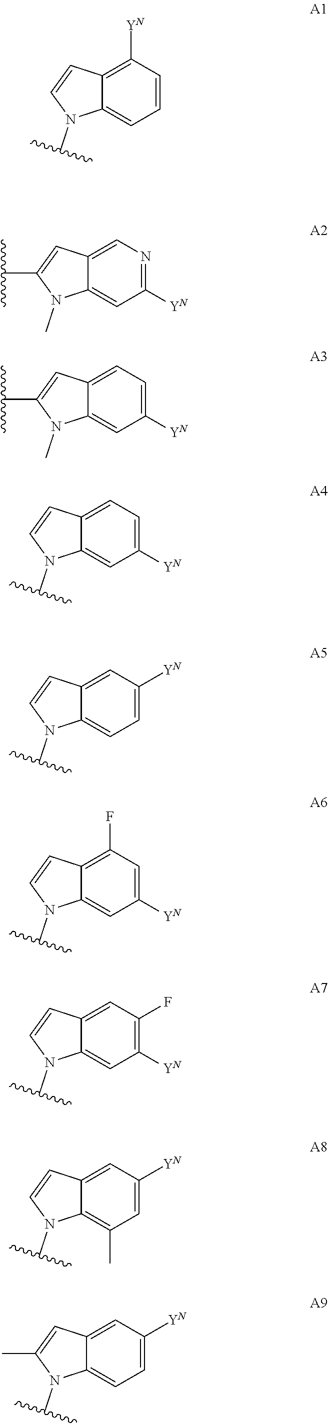

In some of these embodiments W is W2-1

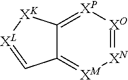

wherein:
X^{K} and X^{L} are either N-linker and CH, N-linker and CMe, or NMe and C-linker respectively;
1 of X^{M} and X^{O} is C-2,4-dioxohexahydropyrimidin-1-yl (Y^{N});
0 or 1 of X^{M} and X^{N} is C—F;
X^{N} may be N if X^{M} is not C—F;
the remainder of X^{N}, X^{M} and X^{O} are CH;
X^{P} is CH or CMe.

In one embodiment W represents any of the groups 22 to 26, 29 and 31 shown below:

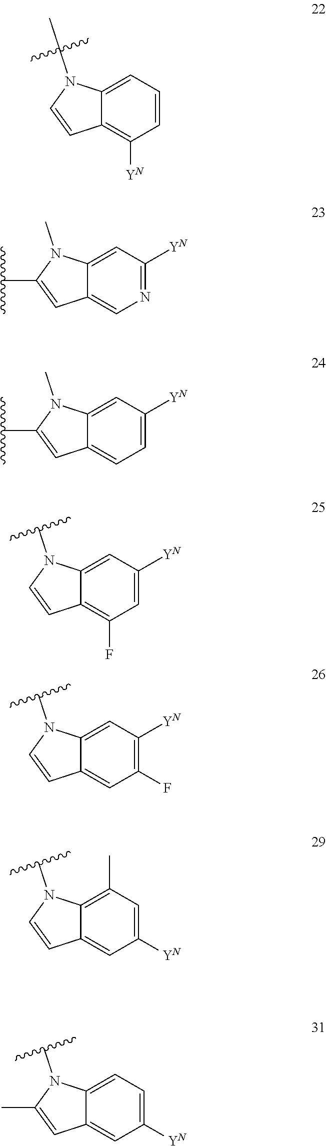

In one embodiment W is W3 which is: —Z^{B}
where Z^{B} is:

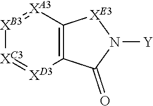

X^{E3} is selected from C(═O) and CR^{E1}R^{E2}, where R^{E1} and R^{E2} are independendently selected from the group consisting of H and C₁₋₃ alkyl, or R^{E1} and R^{E2} taken together with the carbons to be they are attached form a C₃₋₆ cycloalkane ring;
Y is N-(2,6-dioxopiperidin-3-yl);
all of X^{A3}, X^{B3}, X^{C3} and X^{D3} are C;
one pair of X^{A3} and X^{B3}, X^{B3} and X^{C3} and X^{C3} and X^{D3} form a five or six membered fully unsaturated or partially unsaturated N containing heterocylic ring, which is attached to the ‘b’ end of the Linker via a N atom;
the others of X^{A3}, X^{B3}, X^{C3} and X^{D3} bear a group RB, where each RB is selected from H, F, Cl, C₁₋₃ alkyl and C₁₋₃ alkoxy.

In one embodiment X^{E3} is C(═O).

In one embodiment X^{E3} is CR^{E1}R^{E2}, where R^{E1} and R^{E2} are independendently selected from the group consisting of H and C₁₋₃ alkyl, or R^{E1} and R^{E2} taken together with the carbons to be they are attached form a C₃₋₆ cycloalkane ring.

In one embodiment X^{E3} is CR^{E1}R^{E2}, where R^{E1} and R^{E2} are independendently selected from the group consisting of H and C₁₋₃ alkyl, such as methyl.

In one embodiment X^{A3} and X^{B3} form a five or six membered fully unsaturated or partially unsaturated N containing heterocylic ring, which is attached to the ‘b’ end of the Linker via a N atom.

In one embodiment X^{B3} and X^{C3} form a five or six membered fully unsaturated or partially unsaturated N containing heterocylic ring, which is attached to the ‘b’ end of the Linker via a N atom.

In one embodiment X^{C3} and X^{D3} form a five or six membered fully unsaturated or partially unsaturated N containing heterocylic ring, which is attached to the ‘b’ end of the Linker via a N atom.

In one embodiment, the five or six membered fully unsaturated or partially unsaturated N containing heterocylic ring is a five membered ring, where there is no additional unsaturation.

In one embodiment, each R^{B} is selected from H, F, Cl, methyl and methoxy.

In one embodiment, each R^{B} is H.

In one embodiment, W is the group 28:

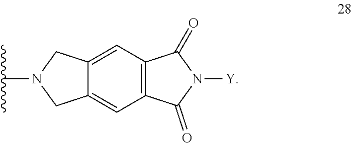

In one embodiment W is W4 which is: —Z^{C}
where Z^{C} is:

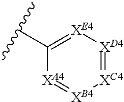

One of X^{B4} and X^{C4} is C—C(═O)—NH—Y;

One of X^{A4}, X^{B4}, X^{C4}, X^{D4} and X^{E4} may be C—F; or

One or two of X^{A4}, X^{B4}, X^{C4}, X^{D4} and X^{E4} may be N;
the remainder of X^{A4}, X^{B4}, X^{C4}, X^{D4} and X^{E4} are CH.

In one embodiment one of X^{A4}, X^{B4}, X^{C4}, X^{D4} and X^{E4} is C—F.

In one embodiment X^{B4} is C—F.

In one embodiment one of X^{B4} is C—F and X^{C4} is C—C(═O)—NH—Y.

In one embodiment two of X^{A4}, X^{B4}, X^{C4}, X^{D4} and X^{E4} are N.

In one embodiment one of X^{A4}, X^{B4}, X^{C4}, X^{D4} and X^{E4} is N.

In one embodiment none of X^{A4}, X^{B4}, X^{C4}, X^{D4} and X^{E4} are N.

In one embodiment none of X^{A4}, X^{B4}, X^{C4}, X^{D4} and X^{E4} are N, and one of X^{A4}, X^{B4}, X^{C4}, X^{D4} and X^{E4} is C—F.

In one embodiment X^{B4} is C—F, X^{C4} is C—C(═O)—NH—Y and X^{A4}, X^{D4} and X^{E4} are CH.

In one embodiment W is the group 30:

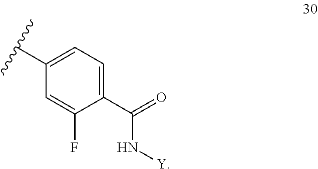

In a further aspect of the specification Formula (Ia) is selected from any one or more of the following groups (1) to (43):
(1) 4-[1-(4-chloro-3-cyano-1H-indol-7-yl)-4-piperidyl]phenyl;
(2) 4-[1-(3-cyano-4-fluoro-1H-indol-7-yl)-4-piperidyl]phenyl;
(3) 4-[1-(3-cyano-4-methyl-1H-indol-7-yl)-4-piperidyl]phenyl;
(4) 4-[1-(4-chloro-3-cyano-1H-indazol-7-yl)-3-piperidyl]phenyl;
(5) 4-[1-(4-chloro-3-cyano-1H-indol-7-yl)-3-piperidyl]phenyl;
(6) 4-[1-(3-cyano-4-fluoro-1H-indol-7-yl)-3-piperidyl]phenyl;
(7) 4-[1-(3-cyano-4-methyl-1H-indol-7-yl)-4-piperidyl]-3-fluoro-phenyl;
(8) 4-[1-(3-cyano-1H-indol-7-yl)-4-piperidyl]phenyl;
(9) 6-[1-(4-chloro-3-cyano-1H-indol-7-yl)-4-piperidyl]-3-pyridyl;
(10) 5-[1-(4-chloro-3-cyano-1H-indol-7-yl)-4-piperidyl]pyrazin-2-yl;
(11) 4-[3-(3-cyano-4-methyl-1H-indol-7-yl)-1-piperidyl]phenyl;
(12) 3-[3-(3-cyano-4-methyl-1H-indol-7-yl)-1-piperidyl]phenyl;
(13) 4-[3-(4-chloro-3-cyano-1H-indol-7-yl)-1-piperidyl]phenyl;
(14) 4-[4-(3-cyano-4-methyl-1H-indol-7-yl)piperazin-1-yl]phenyl;
(15) 5-[1-(4-chloro-3-cyano-1H-indol-7-yl)-4-piperidyl]-2-pyridyl;
(16) 4-[1-(4-chloro-3-cyano-1-methyl-indol-7-yl)-4-piperidyl]phenyl;
(17) 3-[1-(4-chloro-3-cyano-1H-indol-7-yl)-4-piperidyl]phenyl;
(18) 5-[1-(4-chloro-3-cyano-1H-indol-7-yl)-3-piperidyl]-2-pyridyl;
(19) 4-[4-(4-chloro-3-cyano-1H-indol-7-yl)morpholin-2-yl]phenyl;
(20) 4-[4-(4-chloro-3-cyano-1H-indazol-7-yl)morpholin-2-yl]phenyl;
(21) 6-[1-(4-chloro-3-cyano-1H-indol-7-yl)-3-piperidyl]pyridazin-3-yl;
(22) 4-[1-(3-cyano-4-fluoro-1H-indazol-7-yl)-3-piperidyl]phenyl;
(23) 4-[1-(4-chloro-3-cyano-1H-indazol-7-yl)-4-piperidyl]phenyl;
(24) 4-[1-(3-cyano-4-fluoro-1H-indazol-7-yl)-4-piperidyl]phenyl;
(25) 4-[1-(3-cyano-4-methyl-1H-indazol-7-yl)-4-piperidyl]phenyl;
(26) 4-[1-(3-cyano-4-methyl-1H-indol-7-yl)pyrrolidin-3-yl]phenyl;
(27) 4-[[1-(4-chloro-3-cyano-1H-indazol-7-yl)-4-piperidyl]carbamoyl]-3-fluoro-phenyl;
(28) 4-[[1-(4-chloro-3-cyano-1H-indol-7-yl)-4-piperidyl]carbamoyl]-3-fluoro-phenyl;
(29) 4-[[1-(4-chloro-3-cyano-1H-indol-7-yl)-4-piperidyl]carbamoyl]-3-cyano-phenyl;
(30) 4-[[1-(4-chloro-3-cyano-1H-indol-7-yl)-4-piperidyl]carbamoyl]-3-fluoro-5-methyl-phenyl;
(31) 4-[[1-(4-chloro-3-cyano-1H-indol-7-yl)-4-piperidyl]carbamoyl]-2-methyl-phenyl;
(32) 4-[[1-(4-chloro-3-cyano-1H-indol-7-yl)-4-piperidyl]carbamoyl]-2-cyano-phenyl;
(33) 6-[[1-(4-chloro-3-cyano-1H-indol-7-yl)-4-piperidyl]carbamoyl]-3-pyridyl;
(34) 6-[[1-(4-chloro-3-cyano-1H-indazol-7-yl)-4-piperidyl]carbamoyl]-3-pyridyl;
(35) 6-[[1-(3-cyano-4-methyl-1H-indol-7-yl)-4-piperidyl]carbamoyl]-3-pyridyl;
(36) 4-[[1-(4-chloro-3-cyano-1H-indol-7-yl)-4-piperidyl]carbamoyl]phenyl;
(37) 4-[[4-(4-chloro-3-cyano-1H-indol-7-yl)cyclohexyl]carbamoyl]-3-fluoro-phenyl;
(38) 4-[[1-(4-chloro-3-cyano-1H-indol-7-yl)-4-piperidyl]carbamoyl]-3,5-difluoro-phenyl;
(39) 5-[[1-(4-chloro-3-cyano-1H-indol-7-yl)-4-piperidyl]carbamoyl]pyrazin-2-yl;
(40) 2-[[1-(4-chloro-3-cyano-1H-indol-7-yl)-4-piperidyl]carbamoyl]pyrimidin-5-yl;
(41) 4-[[1-(4-chloro-3-cyano-1H-indol-7-yl)-4-piperidyl]carbamoyl]-3-methyl-phenyl;
(42) 6-[[1-(4-chloro-3-cyano-1H-indol-7-yl)-4-piperidyl]carbamoyl]pyridazin-3-yl;
(43) 6-[[(3R)-1-(4-chloro-3-cyano-1H-indol-7-yl)-3-piperidyl]carbamoyl]-3-pyridyl.

In a further aspect of the specification there is provided a PROTAC compound or a pharmaceutically acceptable salt thereof, containing an E3 ubiquitin ligase cereblon binder unit and an AR binding unit of Formula (Ia) or Formula (Ib):

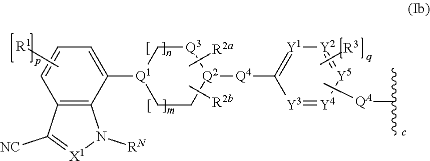

where:
Q^{A} is -G-Q^{H}- or -G-(C₁₋₅alkylene)-;
G is a direct bond, —CH₂—, —C(═O)—, —O—, or —N(R^{G})— where each R^{G} is independently H or C₁₋₃alkyl;
Q^{H} is a 4-12-membered nitrogen-containing saturated or partially unsaturated heterocyclic group;
and where R¹, p, X¹, R^{N}, n, m, R^{2a}, R^{ab}, Q¹, Q², Q³, Q⁴, Y¹, Y², Y³, Y⁴, Y⁵, R³ and q may take any of the values defined herein for each of these groups/variables respectively.

In one embodiment Q^{H} is a 4-12-membered nitrogen-containing saturated heterocyclic group.

For avoidance of doubt

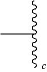

in Formula (Ib) indicates a point of connection via a single covalent bond to the remainder of the PROTAC compound.

In a further aspect of the specification there is provided a PROTAC compound or a pharmaceutically acceptable salt thereof, containing an E3 ubiquitin ligase cereblon binder unit linked to an AR binding unit of Formula (Ia) of Formula (Ib), as defined herein.

In further embodiments of this specification Formula (Ib) may be:

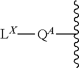

where L^{X} may be any of the groups (1) to (43) listed hereinabove in connection with Formula (Ia); and where Q^{A} may take any value(s) disclosed herein for Q^{A}.

In a further aspect of the specification there is provided a PROTAC compound or a pharmaceutically acceptable salt thereof, containing an E3 ubiquitin ligase cereblon binder unit and an AR binding unit of Formula (Ia) of Formula (Ic):

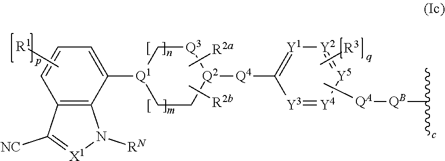

where:
Q^{A} is -G-Q^{H}- or -G-(C₁₋₅alkylene)-;
G is a direct bond, —CH₂—, —C(═O)—, —O—, or —N(R^{G})— where R^{G} is H or C₁₋₃alkyl;
Q^{B} is a direct bond, -Q^{B1}-Q^{B2}-Q^{B3}- or C₁₋₃alkylene optionally substituted by one or more F (e.g. 1 or 2);
   where:
   Q^{B1} & Q^{B3} each independently represent a direct bond or C₁₋₂alkylene;
   Q^{B2} is Q^{H}, —O—CH₂CH₂—O—, —O— or —N(R^{J})— where R^{J} is H or C₁₋₃alkyl;
and each Q^{H} is independently a 4-12-membered nitrogen-containing saturated or partially unsaturated heterocyclic group;
where the values of Q^{A} and Q^{B} are selected so that Formula (Ic) does not contain any N—N or N—O bonds; and where R¹, p, X¹, R^{N}, n, M, R^{2a}, R^{2b}, Q¹, Q², Q³, Q⁴, Y¹, Y², Y³, Y⁴, Y⁵, R³ and q may otherwise take any of the values defined herein for each of these groups/variables respectively.

In one embodiment Q^{H} is a 4-12-membered nitrogen-containing saturated heterocyclic group. For avoidance of doubt

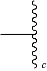

in Formula (Ic) indicates a point of connection via a single covalent bond to the remainder of the PROTAC compound.

In a further aspect of the specification there is provided a PROTAC compound or a pharmaceutically acceptable salt thereof, containing an E3 ubiquitin ligase cereblon binder unit linked to an AR binding unit of Formula (Ia) or Formula (Ic), as defined herein.

In further embodiments of this specification Formula (Tb) may be:

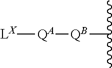

where L^{X} may be any of the groups (1) to (43) listed hereinabove in connection with Formula (Ia); and where Q^{A} and Q^{B} may take any value(s) disclosed herein for Q^{A} and Q^{B}.

In further embodiments there is/are provided compound(s) or pharmaceutically acceptable salt thereof wherein said compound(s) is/are selected from one or more of the “Examples” listed hereinafter. Thus, these embodiments include one or more specific Examples (for instance one Example, or two or three specific Examples) selected from the group consisting of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 153, 164, 165, 166, 167, 168, 169, 170, 171, 172, 173, 174, 175, 176, 177, 178, 179, 180 and 181.

It is to be understood that the compound of an Example listed hereinafter relates to the title compound name, and is not limited in any way by the method of preparation nor whether a given compound was isolated in the form of a salt rather than as a neutral molecule.

In some embodiments there is provided a compound of Formula (VI):

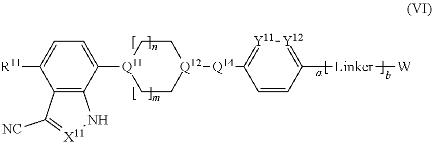

or a pharmaceutically acceptable salt thereof,
wherein:
X¹¹ is CH or N;
R¹¹ is F, Cl or Me;
Q¹¹ and Q¹² are either N and CH or CH and N respectively;
n and m are either 1 and 1 or 2 and 0 respectively;
Q¹⁴ is a single bond or —NHC(═O);
Y¹¹ and Y¹² are selected from CH and CH, CF and CH, N and CH, and N and N respectively;
Linker is selected from linkers 3, 47, 49, 54 and 58:

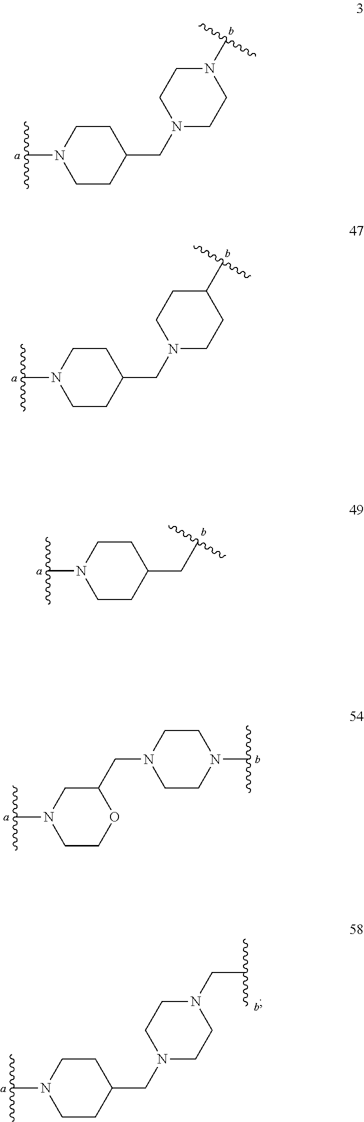 [SMILES: *N1CCC(CN2CCN(*)CC2)CC1 ; *C1CCN(CC2CCN(*)CC2)CC1 ; *CC1CCN(*)CC1 ; *N1CCN(CC2CN(*)CCO2)CC1 ; *CN1CCN(CC2CCN(*)CC2)CC1]

and
W is selected from groups 1, 2, 3, 15, 16, 22, 24, 25, 26, 28, 31 and 32:

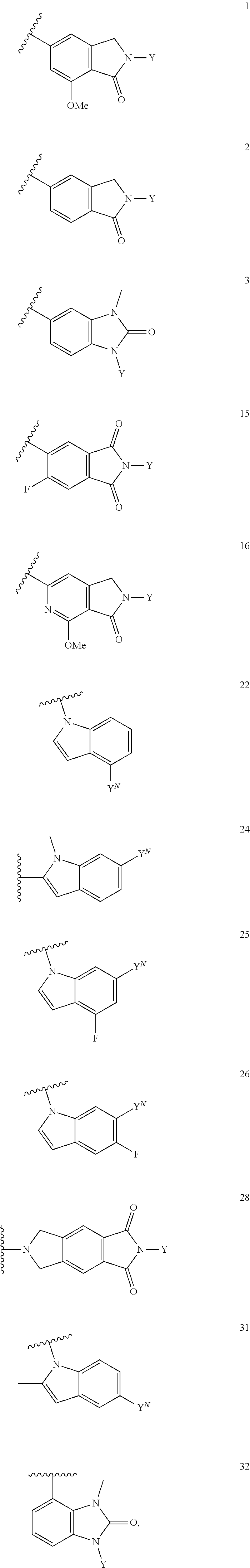

where Y is 2,6-dioxopiperidin-3-yl and Y^{N} is 2,4-dioxohexahydropyrimidin-1-yl.

In some embodiments there is provided a compound of Formula (VI-1):

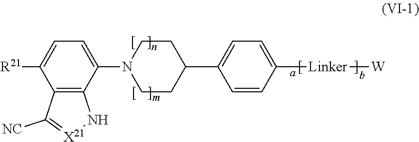

or a pharmaceutically acceptable salt thereof,
wherein:
X²¹ is CH or N;
R²¹ F and Cl;
n and m are either 1 and 1 or 2 and 0 respectively;
Linker is selected from linkers 3 and 47:

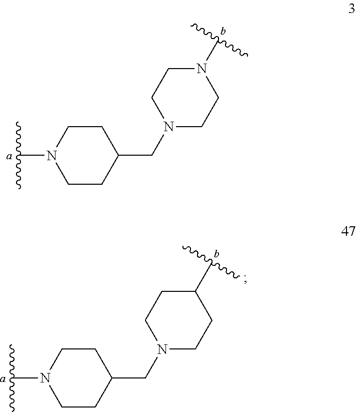 [SMILES: *N1CCC(CN2CCN(*)CC2)CC1 ; *C1CCN(CC2CCN(*)CC2)CC1]

and
W is selected from groups 2, 22 and 24:

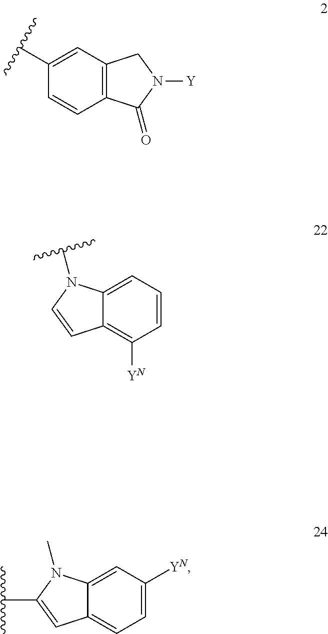

where Y is 2,6-dioxopiperidin-3-yl and Y^{N} is 2,4-dioxohexahydropyrimidin-1-yl.

In some embodiments there is provided a compound of Formula (VI-2):

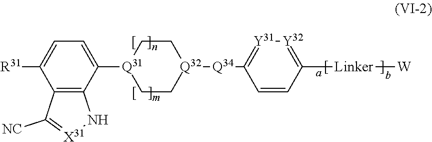

or a pharmaceutically acceptable salt thereof,
wherein:
X³¹ is CH or N;
R³¹ F, Cl and methyl;
n and m are either 1 and 1 or 2 and 0 respectively;
Q³¹ and Q³² are either N and CH or CH and N respectively;
Q³⁴ is a single bond or —NHC(═O);
Y³¹ and Y³² are selected from CH and CH, CF and CH, N and CH, and N and N respectively;
Linker is selected from linkers 3, 47, 49, 54 and 58:

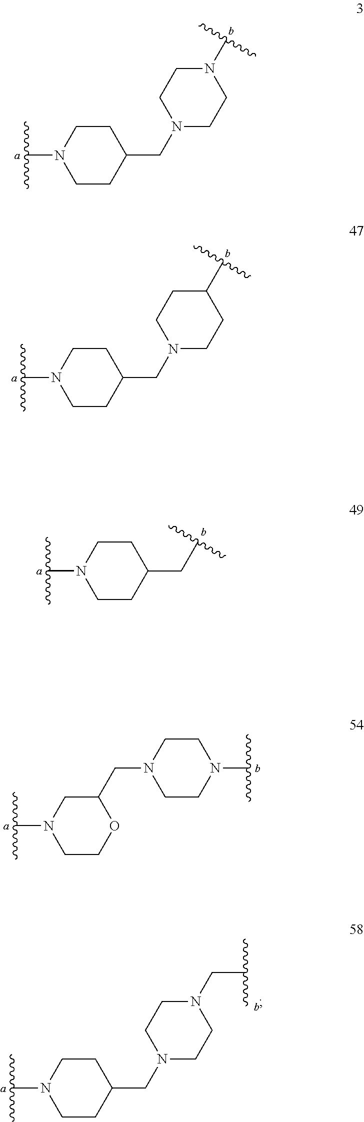 [SMILES: *N1CCC(CN2CCN(*)CC2)CC1 ; *C1CCN(CC2CCN(*)CC2)CC1 ; *CC1CCN(*)CC1 ; *N1CCN(CC2CN(*)CCO2)CC1 ; *CN1CCN(CC2CCN(*)CC2)CC1]

and
W is selected from groups 1, 2, 3, 15, 16, 22, 24, 25, 26, 28, 31 and 32:

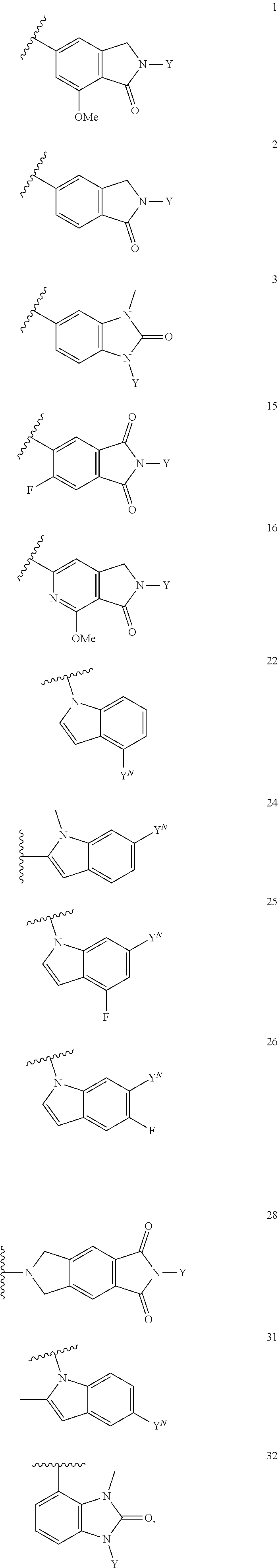

where Y is 2,6-dioxopiperidin-3-yl and Y^{N} is 2,4-dioxohexahydropyrimidin-1-yl.

In some embodiments there is provided a compound of Formula (VII):

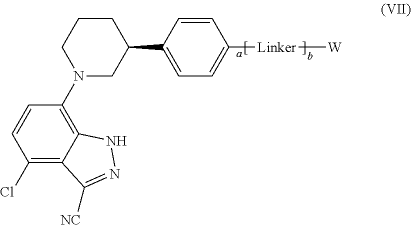

or a pharmaceutically acceptable salt thereof,
wherein:
Linker is a saturated or a partially or fully unsaturated framework comprising C and H atoms and at least one heteroatom, wherein said framework has end points of attachment ‘a’ and ‘b’ and a minimum length of from 5 to 26 atoms between ‘a’ and ‘b’; wherein said framework may include one or more straight and/or branched chains and/or rings and is optionally substituted on any available C atom(s) by one or more F; and
W is an E3 ubiquitin ligase cereblon binder unit.

In some embodiments there is provided a compound of Formula (VII-I):

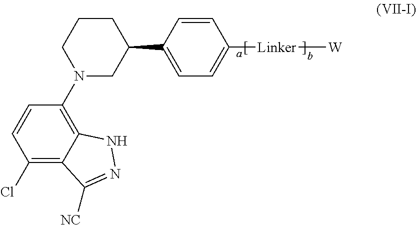

or a pharmaceutically acceptable salt thereof,
wherein:
Linker is selected from linkers 3, 47, 49, 54 and 58:

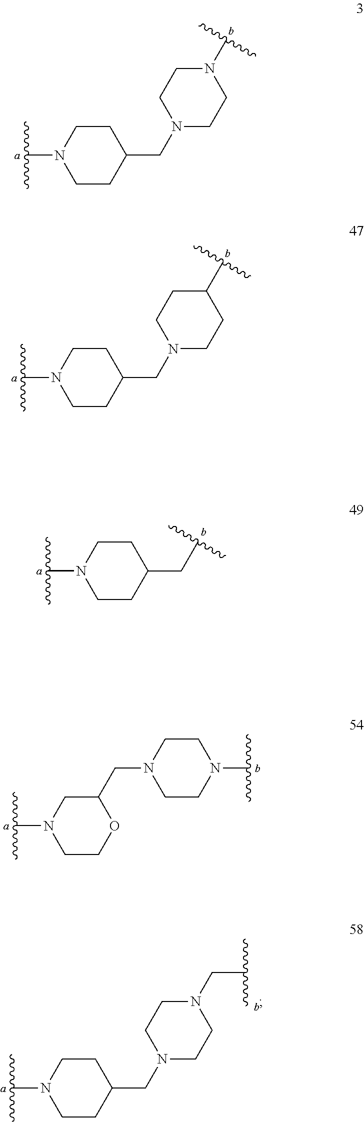 [SMILES: *N1CCC(CN2CCN(*)CC2)CC1 ; *C1CCN(CC2CCN(*)CC2)CC1 ; *CC1CCN(*)CC1 ; *N1CCN(CC2CN(*)CCO2)CC1 ; *CN1CCN(CC2CCN(*)CC2)CC1]

and
W is selected from groups 1, 2, 3, 15, 16, 22, 24, 25, 26, 28, 31 and 32:

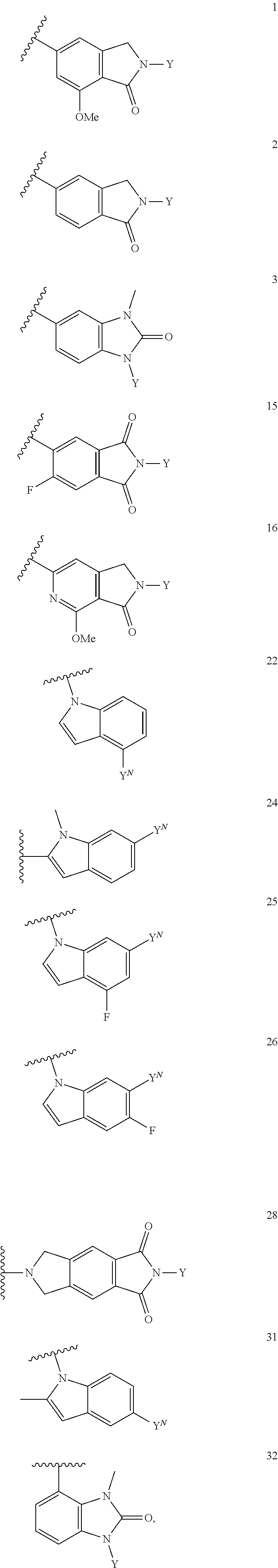

where Y is 2,6-dioxopiperidin-3-yl and Y^{N} is 2,4-dioxohexahydropyrimidin-1-yl.

In some embodiments there is provided a compound of Formula (VII-II):

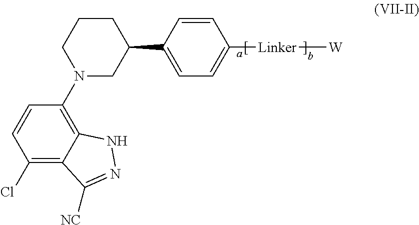

or a pharmaceutically acceptable salt thereof,
wherein:
Linker is selected from linkers 3 and 47:

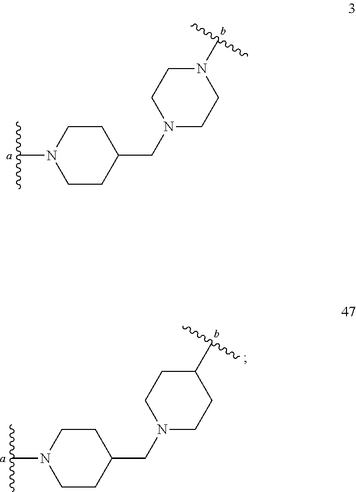 [SMILES: *N1CCC(CN2CCN(*)CC2)CC1 ; *C1CCN(CC2CCN(*)CC2)CC1]

and
W is selected from groups 2, 22 and 24:

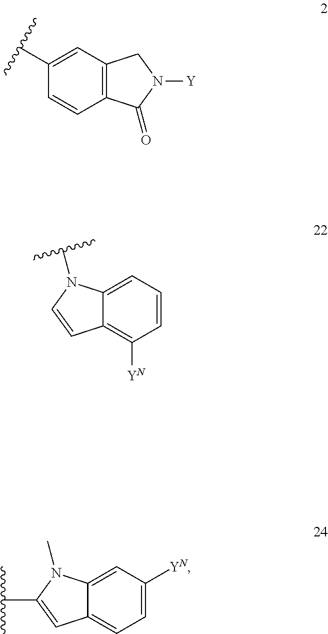

where Y is 2,6-dioxopiperidin-3-yl and Y^{N} is 2,4-dioxohexahydropyrimidin-1-yl.

In some embodiments there is provided a compound of Formula (VII-III):

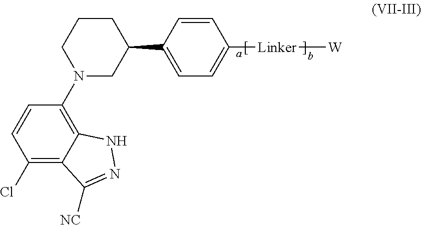

or a pharmaceutically acceptable salt thereof,
wherein:
Linker is selected from linkers 3, 47, 49, 54 and 58:

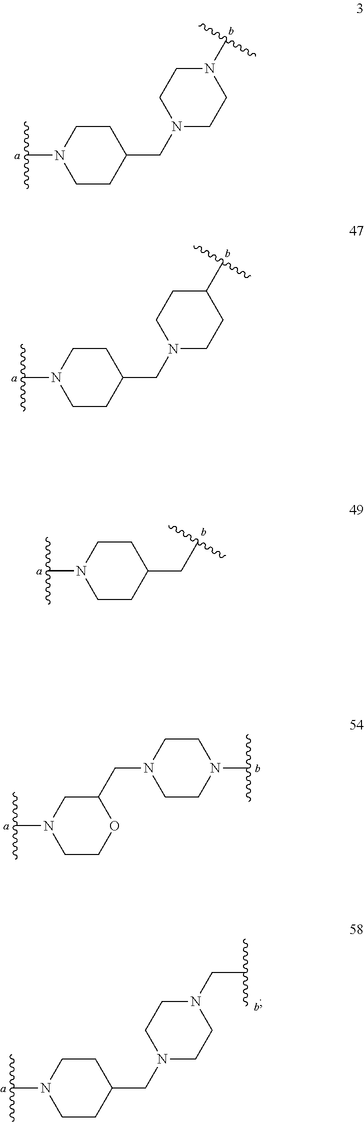 [SMILES: *N1CCC(CN2CCN(*)CC2)CC1 ; *C1CCN(CC2CCN(*)CC2)CC1 ; *CC1CCN(*)CC1 ; *N1CCN(CC2CN(*)CCO2)CC1 ; *CN1CCN(CC2CCN(*)CC2)CC1]

and
W is selected from groups 1, 2, 3, 15, 16, 22, 24, 25, 26, 28, 31 and 32:

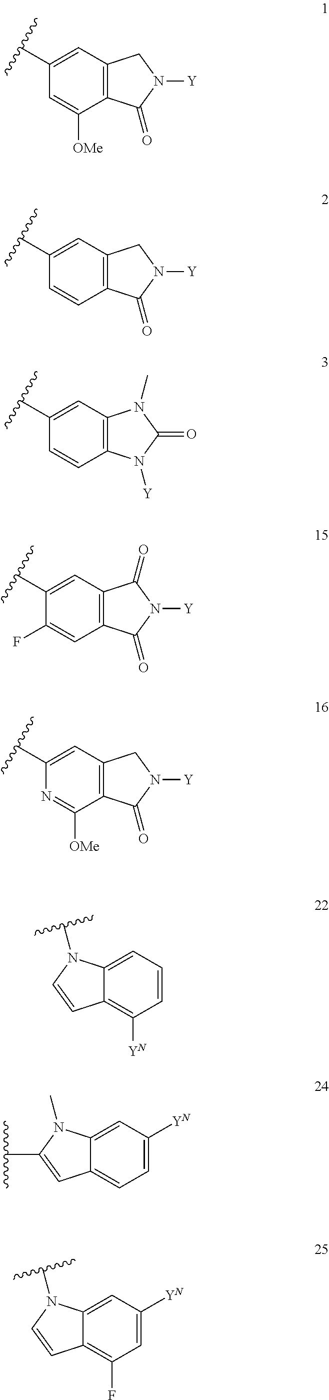

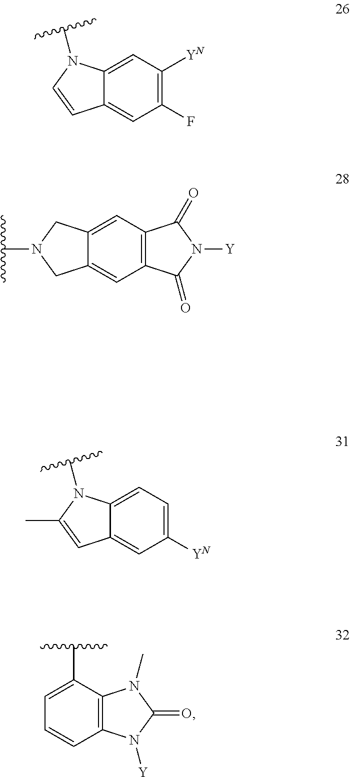

where Y is 2,6-dioxopiperidin-3-yl and Y^{N} is 2,4-dioxohexahydropyrimidin-1-yl.

In some embodiments there is provided a compound of Formula (VIII):

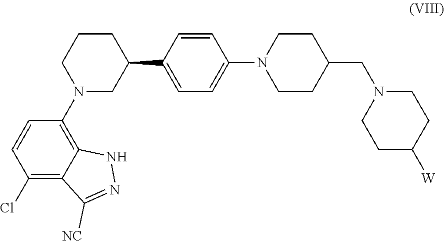

or a pharmaceutically acceptable salt thereof,
wherein:
W is an E3 ubiquitin ligase cereblon binder unit.

In some embodiments there is provided a compound of Formula (VIII-I):

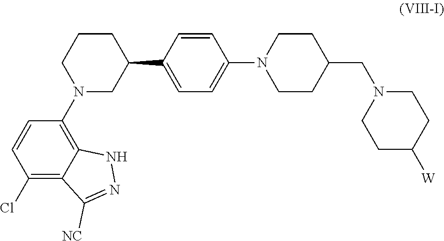

or a pharmaceutically acceptable salt thereof,
wherein:
W is selected from groups 1, 2, 3, 15, 16, 22, 24, 25, 26, 28, 31 and 32:

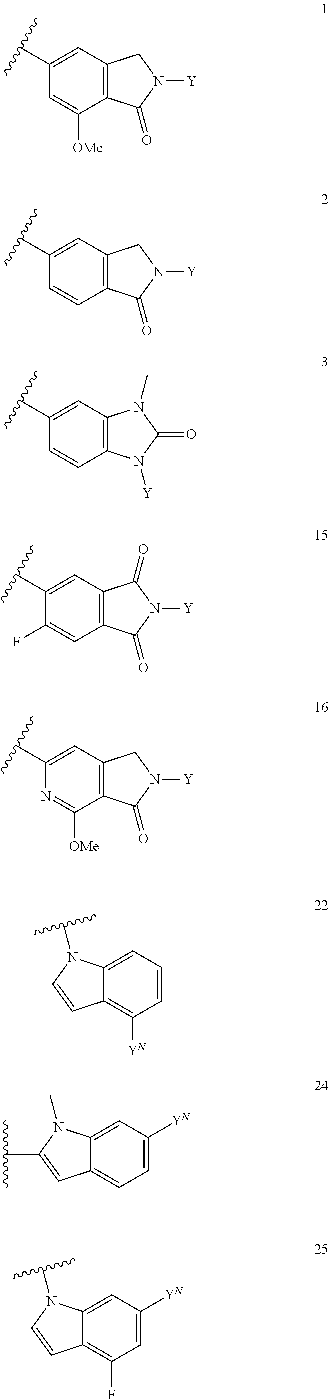

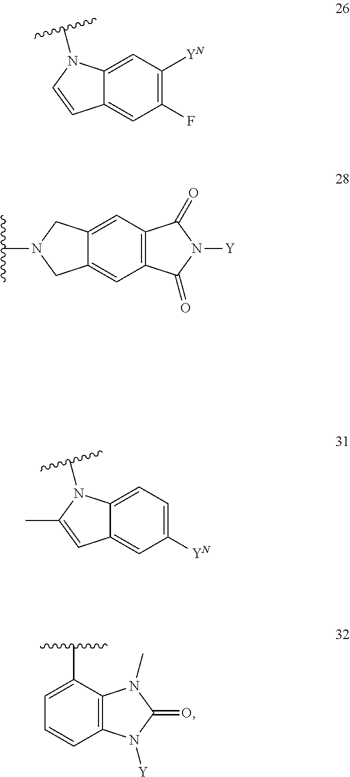

where Y is 2,6-dioxopiperidin-3-yl and Y^{N} is 2,4-dioxohexahydropyrimidin-1-yl.

In some embodiments there is provided a compound of Formula (VIII-II):

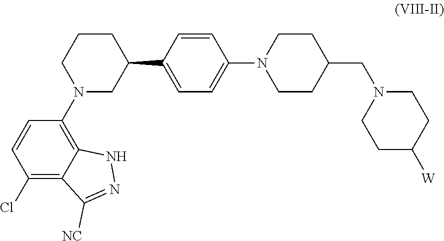

or a pharmaceutically acceptable salt thereof,
wherein:
W is selected from groups 2, 22 and 24:

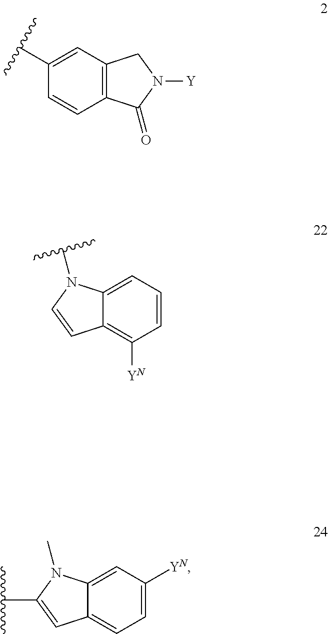

where Y is 2,6-dioxopiperidin-3-yl and Y^{N} is 2,4-dioxohexahydropyrimidin-1-yl.

Specific compounds of this specification include:
4-Chloro-7-(4-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-methyl-1H-indole-3-carbonitrile;
4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile;
4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indazole-3-carbonitrile;
4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile;
4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indole-3-carbonitrile;
4-Chloro-7-[4-(4-{4-[(4-{2-[(3R*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3R*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-[(3R)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile;
7-(4-{4-[4-({1-[2-(2,6-Dioxopiperidin-3-yl)-1-oxo-2,3-dihydro-1H-isoindol-5-yl]piperidin-4-yl}methyl)piperazin-1-yl]phenyl}piperidin-1-yl)-4-fluoro-1H-indole-3-carbonitrile;
7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indole-3-carbonitrile;
7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indole-3-carbonitrile;
4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile;
4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile;
7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[2-(2,6-dioxopiperidin-3-yl)-1-oxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-4-methyl-1H-indole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-methyl-1H-indole-3-carbonitrile;
7-{4-[4-(4-{4-[2-(2,6-Dioxopiperidin-3-yl)-4-methoxy-3-oxo-2,3-dihydro-1H-pyrrolo[3,4-c]pyridin-6-yl]piperazin-1-yl}butoxy)phenyl]piperidin-1-yl}-4-methyl-1H-indole-3-carbonitrile;
4-Chloro-7-{4-[4-(4-{4-[2-(2,6-dioxopiperidin-3-yl)-4-methoxy-3-oxo-2,3-dihydro-1H-pyrrolo[3,4-c]pyridin-6-yl]piperazin-1-yl}butoxy)phenyl]piperidin-1-yl}-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-4-fluoro-1H-indole-3-carbonitrile;
4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indole-3-carbonitrile;
4-Chloro-7-[4-(4-{4-[(4-{2-[(3R*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indole-3-carbonitrile;
4-Chloro-7-[(3S)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile;
4-Chloro-7-[(3S)-3-(4-{4-[(4-{2-[(3R*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
4-Chloro-7-[(3S)-3-(4-{4-[(4-{2-[(3S*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]-2-fluorophenyl}piperidin-1-yl)-4-methyl-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]-2-fluorophenyl}piperidin-1-yl)-4-methyl-1H-indole-3-carbonitrile;
7-(4-{4-[4-({4-[2-(2,6-Dioxopiperidin-3-yl)-7-methoxy-1,3-dioxo-2,3-dihydro-1H-isoindol-5-yl]piperazin-1-yl}methyl)piperidin-1-yl]-2-fluorophenyl}piperidin-1-yl)-4-methyl-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperidin-1-yl}-4-fluoro-1H-indole-3-carbonitrile;
4-Chloro-7-{4-[4-(4-{4-[2-(2,6-dioxopiperidin-3-yl)-1-oxo-2,3-dihydro-1H-isoindol-5-yl]piperazin-1-yl}butoxy)phenyl]piperidin-1-yl}-1H-indole-3-carbonitrile;
4-Chloro-7-{4-[4-(4-{4-[4-(2,4-dioxo-1,3-diazinan-1-yl)-1H-indol-1-yl]piperidin-1-yl}butoxy)phenyl]piperidin-1-yl}-1H-indole-3-carbonitrile;
4-Chloro-7-[(3R)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile;
7-{4-[4-(4-{4-[2-(2,6-Dioxopiperidin-3-yl)-7-methoxy-1,3-dioxo-2,3-dihydro-1H-isoindol-5-yl]piperazin-1-yl}butoxy)phenyl]piperidin-1-yl}-4-methyl-1H-indole-3-carbonitrile;
4-Chloro-7-(4-{4-[4-({4-[2-(2,6-dioxopiperidin-3-yl)-7-methoxy-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-{4-[4-(4-{4-[2-(2,6-Dioxopiperidin-3-yl)-6-fluoro-1,3-dioxo-2,3-dihydro-1H-isoindol-5-yl]piperazin-1-yl}butoxy)phenyl]piperidin-1-yl}-4-methyl-1H-indole-3-carbonitrile;
4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[2-(2,6-Dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-[(3S)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-[(3R)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-(4-{4-[4-({4-[2-(2,6-dioxopiperidin-3-yl)-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-{4-[4-(4-{4-[2-(2,6-Dioxopiperidin-3-yl)-1,3-dioxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperidin-1-yl}-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-1H-indole-3-carbonitrile;
4-Chloro-7-(4-{5-[4-({4-[2-(2,6-dioxopiperidin-3-yl)-7-methoxy-1-oxo-2,3-dihydro-1H-isoindol-5-yl]piperazin-1-yl}methyl)piperidin-1-yl]pyridin-2-yl}piperidin-1-yl)-1H-indole-3-carbonitrile;
4-Chloro-7-(4-{5-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]pyridin-2-yl}piperidin-1-yl)-1H-indole-3-carbonitrile;
4-Chloro-7-(4-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl)-1H-indole-3-carbonitrile;
4-Chloro-7-(4-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl)-1H-indole-3-carbonitrile;
4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridazin-3-yl}piperidin-1-yl)-1H-indole-3-carbonitrile;
4-Chloro-7-[(3R*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile;
4-Chloro-7-[(3S*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile;
4-Chloro-7-(4-{5-[4-({4-[4-(2,4-dioxo-1,3-diazinan-1-yl)-1H-indol-1-yl]piperidin-1-yl}methyl)piperidin-1-yl]pyrazin-2-yl}piperidin-1-yl)-1H-indole-3-carbonitrile;
4-Chloro-7-(4-{5-[4-({4-[2-(2,6-dioxopiperidin-3-yl)-1-oxo-2,3-dihydro-1H-isoindol-5-yl]piperazin-1-yl}methyl)piperidin-1-yl]pyrazin-2-yl}piperidin-1-yl)-1H-indole-3-carbonitrile;
7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-methyl-1H-indole-3-carbonitrile;
4-Chloro-7-[4-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile;
7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile;
4-Chloro-7-(4-{4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]phenyl}piperidin-1-yl)-1H-indole-3-carbonitrile;
4-Chloro-7-[(3S)-3-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile;
7-[(3S)-3-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile;
4-Chloro-7-{4-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile;
4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indazole-3-carbonitrile;
4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile;
7-{(3R*)-1-[4-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-{(3S*)-1-[4-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-{(3R*)-1-[3-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-{(3S*)-1-[3-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-[(3R*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-4-methyl-1H-indole-3-carbonitrile;
7-[(3S*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-4-methyl-1H-indole-3-carbonitrile;
4-Chloro-7-(4-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl)-1H-indole-3-carbonitrile;
4-Chloro-7-(4-{4-[4-({4-[2-(2,6-dioxopiperidin-3-yl)-1-oxo-2,3-dihydro-1H-isoindol-5-yl]piperazin-1-yl}methyl)piperidine-1-carbonyl]phenyl}piperidin-1-yl)-1H-indole-3-carbonitrile;
4-Chloro-7-{4-[4-(4-{4-[2-(2,6-dioxopiperidin-3-yl)-4-methoxy-3-oxo-2,3-dihydro-1H-pyrrolo[3,4-c]pyridin-6-yl]piperazin-1-yl}piperidin-1-yl)phenyl]piperidin-1-yl}-1H-indole-3-carbonitrile;
4-Chloro-7-(4-{4-[4-(2-{4-[2-(2,6-dioxopiperidin-3-yl)-4-methoxy-3-oxo-2,3-dihydro-1H-pyrrolo[3,4-c]pyridin-6-yl]piperazin-1-yl}ethyl)piperidin-1-yl]phenyl}piperidin-1-yl)-1H-indole-3-carbonitrile;
4-Chloro-7-(4-{4-[4-(3-{4-[2-(2,6-dioxopiperidin-3-yl)-4-methoxy-3-oxo-2,3-dihydro-1H-pyrrolo[3,4-c]pyridin-6-yl]piperazin-1-yl}propyl)piperidin-1-yl]phenyl}piperidin-1-yl)-1H-indole-3-carbonitrile;
7-(4-{4-[4-({4-[5-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[4-({4-[5-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile;
4-Chloro-7-[(3R*)-1-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-3-yl]-1H-indole-3-carbonitrile;
4-Chloro-7-[(3S*)-1-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-3-yl]-1H-indole-3-carbonitrile;
4-Chloro-7-[(3R*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-1H-indole-3-carbonitrile;
4-Chloro-7-[(3S*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperazin-1-yl}-4-methyl-1H-indole-3-carbonitrile;
4-Chloro-7-[(3R*)-1-{4-[1-({1-[2-(2,6-dioxopiperidin-3-yl)-1-oxo-2,3-dihydro-1H-isoindol-5-yl]piperidin-4-yl}methyl)piperidin-4-yl]phenyl}piperidin-3-yl]-1H-indole-3-carbonitrile;
4-Chloro-7-[(3S*)-1-{4-[1-({1-[2-(2,6-dioxopiperidin-3-yl)-1-oxo-2,3-dihydro-1H-isoindol-5-yl]piperidin-4-yl}methyl)piperidin-4-yl]phenyl}piperidin-3-yl]-1H-indole-3-carbonitrile;
4-Chloro-7-[(3R*)-1-{4-[1-({1-[2-(2,6-dioxopiperidin-3-yl)-4-methoxy-3-oxo-2,3-dihydro-1H-pyrrolo[3,4-c]pyridin-6-yl]piperidin-4-yl}methyl)piperidin-4-yl]phenyl}piperidin-3-yl]-1H-indole-3-carbonitrile;
4-Chloro-7-[(3S*)-1-{4-[1-({1-[2-(2,6-dioxopiperidin-3-yl)-4-methoxy-3-oxo-2,3-dihydro-1H-pyrrolo[3,4-c]pyridin-6-yl]piperidin-4-yl}methyl)piperidin-4-yl]phenyl}piperidin-3-yl]-1H-indole-3-carbonitrile;
4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidine-1-carbonyl]phenyl}piperidin-1-yl)-1H-indole-3-carbonitrile;
7-[4-(4-{[1-({1-[2-(2,6-Dioxopiperidin-3-yl)-1-oxo-2,3-dihydro-1H-isoindol-5-yl]piperidin-4-yl}methyl)piperidin-4-yl]oxy}phenyl)piperidin-1-yl]-4-fluoro-1H-indole-3-carbonitrile;
4-Chloro-7-(4-{4-[4-({4-[5-(2,4-dioxo-1,3-diazinan-1-yl)-7-methyl-1H-indol-1-yl]piperidin-1-yl}methyl)piperidin-1-yl]phenyl}piperidin-1-yl)-1H-indole-3-carbonitrile;
4-Chloro-7-(4-{4-[4-({4-[6-(2,4-dioxo-1,3-diazinan-1-yl)-1-methyl-1H-pyrrolo[3,2-b]pyridin-2-yl]piperidin-1-yl}methyl)piperidin-1-yl]phenyl}piperidin-1-yl)-1H-indole-3-carbonitrile;
4-{4-[(1-{4-[(3S)-1-(4-chloro-3-cyano-1H-indol-7-yl)piperidin-3-yl]phenyl}piperidin-4-yl)methyl]piperazin-1-yl}-N-(2,6-dioxopiperidin-3-yl)-2-fluorobenzamide;
4-{4-[(1-{4-[1-(3-Cyano-4-fluoro-1H-indol-7-yl)piperidin-4-yl]phenyl}piperidin-4-yl)methyl]piperazin-1-yl}-N-(2,6-dioxopiperidin-3-yl)-2-fluorobenzamide;
4-{4-[(1-{4-[1-(4-Chloro-3-cyano-1H-indol-7-yl)piperidin-4-yl]phenyl}piperidin-4-yl)methyl]piperazin-1-yl}-N-(2,6-dioxopiperidin-3-yl)-2-fluorobenzamide;
7-[4-(4-{[4-({1-[2-(2,6-Dioxopiperidin-3-yl)-1-oxo-2,3-dihydro-1H-isoindol-5-yl]piperidin-4-yl}methyl)piperazin-1-yl]methyl}phenyl)piperidin-1-yl]-4-fluoro-1H-indole-3-carbonitrile;
4-Chloro-7-(4-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl)-1H-indole-3-carbonitrile;
4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridin-3-yl}piperidin-1-yl)-1H-indole-3-carbonitrile;
4-Chloro-7-{4-[4-(4-{4-[2-(2,6-dioxopiperidin-3-yl)-1-oxo-2,3-dihydro-1H-isoindol-5-yl]piperazin-1-yl}piperidin-1-yl)phenyl]piperidin-1-yl}-1H-indole-3-carbonitrile;
4-Chloro-7-[4-(4-{4-[4-(2,4-dioxo-1,3-diazinan-1-yl)-1H-indol-1-yl][1,4′-bipiperidin]-1′-yl}phenyl)piperidin-1-yl]-1H-indole-3-carbonitrile;
4-chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1-methyl-1H-indole-3-carbonitrile;
4-Chloro-7-(4-{3-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile;
4-Chloro-7-[(3S)-3-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl]-1H-indole-3-carbonitrile;
4-Chloro-7-[(3S*)-3-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl]-1H-indole-3-carbonitrile;
4-Chloro-7-[(3R*)-3-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl]-1H-indole-3-carbonitrile;
4-Chloro-7-[(2S*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indole-3-carbonitrile;
4-Chloro-7-[(2R*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indole-3-carbonitrile;
4-Chloro-7-[(2S*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indazole-3-carbonitrile;
4-Chloro-7-[(2R*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indazole-3-carbonitrile;
4-Chloro-7-[(3R*)-3-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl]-1H-indole-3-carbonitrile;
4-Chloro-7-[(3S*)-3-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl]-1H-indole-3-carbonitrile;
4-Chloro-7-(4-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl)-1H-indole-3-carbonitrile;
4-Chloro-7-(4-{2-[4-({4-[2-(2,6-dioxopiperidin-3-yl)-1-oxo-2,3-dihydro-1H-isoindol-5-yl]piperazin-1-yl}methyl)piperidin-1-yl]pyrimidin-5-yl}piperidin-1-yl)-1H-indole-3-carbonitrile;
4-Chloro-7-(4-{2-[4-({4-[4-(2,4-dioxo-1,3-diazinan-1-yl)-1H-indol-1-yl]piperidin-1-yl}methyl)piperidin-1-yl]pyrimidin-5-yl}piperidin-1-yl)-1H-indole-3-carbonitrile;
7-(4-{4-[(2R)-2-({4-[2-(2,6-dioxopiperidin-3-yl)-1-oxo-2,3-dihydro-1H-isoindol-5-yl]piperazin-1-yl}methyl)morpholin-4-yl]phenyl}piperidin-1-yl)-4-fluoro-1H-indole-3-carbonitrile;
4-Chloro-7-(4-{4-[(2R)-2-({4-[2-(2,6-dioxopiperidin-3-yl)-1-oxo-2,3-dihydro-1H-isoindol-5-yl]piperazin-1-yl}methyl)morpholin-4-yl]phenyl}piperidin-1-yl)-1H-indole-3-carbonitrile;
7-(4-{4-[7-({4-[2-(2,6-Dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl)-4-fluoro-1H-indole-3-carbonitrile;
4-Chloro-7-(4-{4-[7-({4-[2-(2,6-dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl)-1H-indole-3-carbonitrile;
4-Chloro-7-[(3S)-3-{4-[7-({4-[2-(2,6-dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl]-1H-indole-3-carbonitrile;
4-Chloro-7-[(3S)-3-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl]-1H-indole-3-carbonitrile;
4-Chloro-7-[(3R)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile;
7-[(3S)-3-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indazole-3-carbonitrile;
7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indazole-3-carbonitrile;
7-(4-{4-[7-({4-[4-(2,4-Dioxo-1,3-diazinan-1-yl)-1H-indol-1-yl]piperidin-1-yl}methyl)-2-azaspiro[3.5]nonan-2-yl]phenyl}piperidin-1-yl)-4-fluoro-1H-indole-3-carbonitrile;
4-Chloro-7-[(3R)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indole-3-carbonitrile;
4-Chloro-7-[(3R)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indazole-3-carbonitrile;
4-Chloro-7-[(3S)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indazole-3-carbonitrile;
7-(4-{4-[4-({4-[6-(2,4-Dioxo-1,3-diazinan-1-yl)-1-methyl-1H-indol-2-yl]piperidin-1-yl}methyl)piperidin-1-yl]phenyl}piperidin-1-yl)-4-fluoro-1H-indazole-3-carbonitrile;
7-(4-{4-[4-({4-[4-(2,4-Dioxo-1,3-diazinan-1-yl)-2-methyl-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl)-1H-indole-3-carbonitrile;
4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indazole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indazole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indazole-3-carbonitrile;
4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indazole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indazole-3-carbonitrile;
7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indazole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indazole-3-carbonitrile;
7-{(3S*)-3-[4-(4-{4-[2-(2,6-Dioxopiperidin-3-yl)-1-oxo-2,3-dihydro-1H-isoindol-5-yl]piperazin-1-yl}butoxy)phenyl]pyrrolidin-1-yl}-4-methyl-1H-indole-3-carbonitrile;
7-{(3R*)-3-[4-(4-{4-[2-(2,6-Dioxopiperidin-3-yl)-1-oxo-2,3-dihydro-1H-isoindol-5-yl]piperazin-1-yl}butoxy)phenyl]pyrrolidin-1-yl}-4-methyl-1H-indole-3-carbonitrile;
N-[1-(4-Chloro-3-cyano-1H-indazol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[5-(2,4-dioxo-1,3-diazinan-1-yl)-1H-indol-1-yl]piperidin-1-yl}methyl)piperidin-1-yl]-2-fluorobenzamide;
N-[1-(4-chloro-3-cyano-1-{H}-indol-7-yl)-4-piperidyl]-4-[4-[[4-[4-[(2,6-dioxo-3-piperidyl)carbamoyl]-3-fluoro-phenyl]piperazin-1-yl]methyl]-1-piperidyl]-2-fluorobenzamide;
N-[1-(4-Chloro-3-cyano-1H-indol-7-yl)piperidin-4-yl]-4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)-2-fluorobenzamide;
N-[1-(4-Chloro-3-cyano-1H-indol-7-yl)piperidin-4-yl]-4-[4-({4-[6-(2,4-dioxo-1,3-diazinan-1-yl)-5-fluoro-1-methyl-1H-indol-2-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]-2-fluorobenzamide;
N-[1-(4-Chloro-3-cyano-1H-indol-7-yl)piperidin-4-yl]-4-[4-({4-[6-(2,4-dioxo-1,3-diazinan-1-yl)-1-methyl-1H-indol-2-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[2-(2,6-dioxopiperidin-3-yl)-1-oxo-2,3-dihydro-1H-isoindol-5-yl]piperazin-1-yl}methyl)piperidin-1-yl]-2-fluorobenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]-2-fluorobenzamide;
N-[1-(4-Chloro-3-cyano-1H-indol-7-yl)piperidin-4-yl]-2-cyano-4-[4-({4-[4-(2,4-dioxo-1,3-diazinan-1-yl)-1H-indol-1-yl]piperidin-1-yl}methyl)piperidin-1-yl]benzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluoro-6-methylbenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-3-methylbenzamide;
N-[1-(4-Chloro-3-cyano-1H-indol-7-yl)piperidin-4-yl]-3-cyano-4-[4-({4-[4-(2,4-dioxo-1,3-diazinan-1-yl)-1H-indol-1-yl]piperidin-1-yl}methyl)piperidin-1-yl]benzamide;
N-[1-(4-Chloro-3-cyano-1H-indol-7-yl)piperidin-4-yl]-5-(4-{4-[2-(2,6-dioxopiperidin-3-yl)-6-fluoro-1,3-dioxo-2,3-dihydro-1H-isoindol-5-yl]piperazin-1-yl}piperidin-1-yl)pyridine-2-carboxamide;
N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyridine-2-carboxamide;
N-[1-(4-Chloro-3-cyano-1H-indol-7-yl)piperidin-4-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide;
N-[1-(4-Chloro-3-cyano-1H-indol-7-yl)piperidin-4-yl]-5-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]pyridine-2-carboxamide;
N-[1-(4-Chloro-3-cyano-1H-indazol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyridine-2-carboxamide;
N-[1-(3-Cyano-4-methyl-1H-indol-7-yl)piperidin-4-yl]-5-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]pyridine-2-carboxamide;
N-[1-(3-Cyano-4-methyl-1H-indol-7-yl)piperidin-4-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]benzamide;
N-[4-(4-Chloro-3-cyano-1H-indol-7-yl)cyclohexyl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2,6-difluorobenzamide;
N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyrazine-2-carboxamide;
N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyrimidine-2-carboxamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-methylbenzamide;
N-[1-(4-Chloro-3-cyano-1H-indol-7-yl)piperidin-4-yl]-6-[4-({4-[4-(2,4-dioxo-1,3-diazinan-1-yl)-1H-indol-1-yl]piperidin-1-yl}methyl)piperidin-1-yl]pyridazine-3-carboxamide;
N-[(3R*)-1-(4-Chloro-3-cyano-1H-indol-7-yl)piperidin-3-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide; and
N-[(3S*)-1-(4-Chloro-3-cyano-1H-indol-7-yl)piperidin-3-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide;
or pharmaceutically acceptable salts thereof.
Specific compounds of this specification include:
4-Chloro-7-(4-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-methyl-1H-indole-3-carbonitrile;
4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile;
4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indazole-3-carbonitrile;
4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile;
4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indole-3-carbonitrile;
4-Chloro-7-[4-(4-{4-[(4-{2-[(3R*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3R*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-[(3R)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile;
7-(4-{4-[4-({1-[2-(2,6-Dioxopiperidin-3-yl)-1-oxo-2,3-dihydro-1H-isoindol-5-yl]piperidin-4-yl}methyl)piperazin-1-yl]phenyl}piperidin-1-yl)-4-fluoro-1H-indole-3-carbonitrile;
7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indole-3-carbonitrile;
7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indole-3-carbonitrile;
4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile;
4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile;
7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[2-(2,6-dioxopiperidin-3-yl)-1-oxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-4-methyl-1H-indole-3-carbonitrile;
or pharmaceutically acceptable salts thereof.
Further specific compounds of this specification include:
4-Chloro-7-(4-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl)-1H-indole-3-carbonitrile;
4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indazole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indazole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indazole-3-carbonitrile;
4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indazole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indazole-3-carbonitrile;
7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indazole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indazole-3-carbonitrile;
7-{(3S*)-3-[4-(4-{4-[2-(2,6-Dioxopiperidin-3-yl)-l-oxo-2,3-dihydro-1H-isoindol-5-yl]piperazin-1-yl}butoxy)phenyl]pyrrolidin-1-yl}-4-methyl-1H-indole-3-carbonitrile;
7-{(3R*)-3-[4-(4-{4-[2-(2,6-Dioxopiperidin-3-yl)-l-oxo-2,3-dihydro-1H-isoindol-5-yl]piperazin-1-yl}butoxy)phenyl]pyrrolidin-1-yl}-4-methyl-1H-indole-3-carbonitrile;
N-[1-(4-Chloro-3-cyano-1H-indazol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[5-(2,4-dioxo-1,3-diazinan-1-yl)-1H-indol-1-yl]piperidin-1-yl}methyl)piperidin-1-yl]-2-fluorobenzamide;
N-[1-(4-chloro-3-cyano-1-{H}-indol-7-yl)-4-piperidyl]-4-[4-[[4-[4-[(2,6-dioxo-3-piperidyl)carbamoyl]-3-fluoro-phenyl]piperazin-1-yl]methyl]-1-piperidyl]-2-fluorobenzamide;
N-[1-(4-Chloro-3-cyano-1H-indol-7-yl)piperidin-4-yl]-4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)-2-fluorobenzamide;
N-[1-(4-Chloro-3-cyano-1H-indol-7-yl)piperidin-4-yl]-4-[4-({4-[6-(2,4-dioxo-1,3-diazinan-1-yl)-5-fluoro-1-methyl-1H-indol-2-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]-2-fluorobenzamide;
N-[1-(4-Chloro-3-cyano-1H-indol-7-yl)piperidin-4-yl]-4-[4-({4-[6-(2,4-dioxo-1,3-diazinan-1-yl)-l-methyl-1H-indol-2-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[2-(2,6-dioxopiperidin-3-yl)-1-oxo-2,3-dihydro-1H-isoindol-5-yl]piperazin-1-yl}methyl)piperidin-1-yl]-2-fluorobenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]-2-fluorobenzamide; and
N-[1-(4-Chloro-3-cyano-1H-indol-7-yl)piperidin-4-yl]-2-cyano-4-[4-({4-[4-(2,4-dioxo-1,3-diazinan-1-yl)-1H-indol-1-yl]piperidin-1-yl}methyl)piperidin-1-yl]benzamide;
   or pharmaceutically acceptable salts thereof.

Further specific compounds of this specification include:
4-Chloro-7-[(3R)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile;
7-{4-[4-(4-{4-[2-(2,6-Dioxopiperidin-3-yl)-7-methoxy-1,3-dioxo-2,3-dihydro-1H-isoindol-5-yl]piperazin-1-yl}butoxy)phenyl]piperidin-1-yl}-4-methyl-1H-indole-3-carbonitrile;
4-Chloro-7-(4-{4-[4-({4-[2-(2,6-dioxopiperidin-3-yl)-7-methoxy-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-{4-[4-(4-{4-[2-(2,6-Dioxopiperidin-3-yl)-6-fluoro-1,3-dioxo-2,3-dihydro-1H-isoindol-5-yl]piperazin-1-yl}butoxy)phenyl]piperidin-1-yl}-4-methyl-1H-indole-3-carbonitrile;
4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[2-(2,6-Dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-[(3S)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-[(3R)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-(4-{4-[4-({4-[2-(2,6-dioxopiperidin-3-yl)-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-{4-[4-(4-{4-[2-(2,6-Dioxopiperidin-3-yl)-1,3-dioxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperidin-1-yl}-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-1H-indole-3-carbonitrile;
4-Chloro-7-(4-{5-[4-({4-[2-(2,6-dioxopiperidin-3-yl)-7-methoxy-1-oxo-2,3-dihydro-1H-isoindol-5-yl]piperazin-1-yl}methyl)piperidin-1-yl]pyridin-2-yl}piperidin-1-yl)-1H-indole-3-carbonitrile;
4-Chloro-7-(4-{5-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]pyridin-2-yl}piperidin-1-yl)-1H-indole-3-carbonitrile;
4-Chloro-7-(4-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl)-1H-indole-3-carbonitrile;
4-Chloro-7-(4-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl)-1H-indole-3-carbonitrile;
4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridazin-3-yl}piperidin-1-yl)-1H-indole-3-carbonitrile;
4-Chloro-7-[(3R*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile;
4-Chloro-7-[(3S*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile;
4-Chloro-7-(4-{5-[4-({4-[4-(2,4-dioxo-1,3-diazinan-1-yl)-1H-indol-1-yl]piperidin-1-yl}methyl)piperidin-1-yl]pyrazin-2-yl}piperidin-1-yl)-1H-indole-3-carbonitrile;
4-Chloro-7-(4-{5-[4-({4-[2-(2,6-dioxopiperidin-3-yl)-1-oxo-2,3-dihydro-1H-isoindol-5-yl]piperazin-1-yl}methyl)piperidin-1-yl]pyrazin-2-yl}piperidin-1-yl)-1H-indole-3-carbonitrile;
7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-methyl-1H-indole-3-carbonitrile;
4-Chloro-7-[4-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile;
7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile;
4-Chloro-7-(4-{4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]phenyl}piperidin-1-yl)-1H-indole-3-carbonitrile;
4-Chloro-7-[(3S)-3-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile;
7-[(3S)-3-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile;
4-Chloro-7-{4-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile;
4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indazole-3-carbonitrile; and
4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile;
or pharmaceutically acceptable salts thereof.

In one embodiment, this specification relates to a compound which is 4-Chloro-7-(4-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile

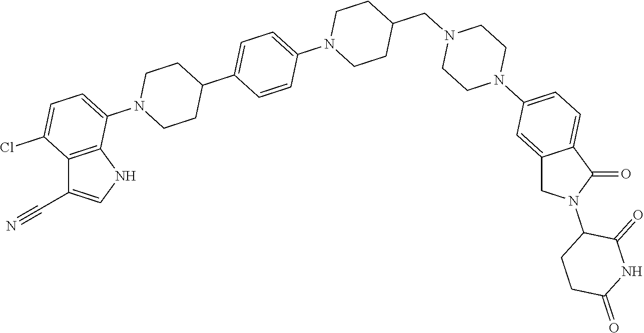 [SMILES: N#Cc1c[nH]c2c(N3CCC(c4ccc(N5CCC(CN6CCN(c7ccc8c(c7)CN(C7CCC(=O)NC7=O)C8=O)CC6)CC5)cc4)CC3)ccc(Cl)c12]

or a pharmaceutically acceptable salt thereof.

In one embodiment, this specification relates to a compound which is 7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile

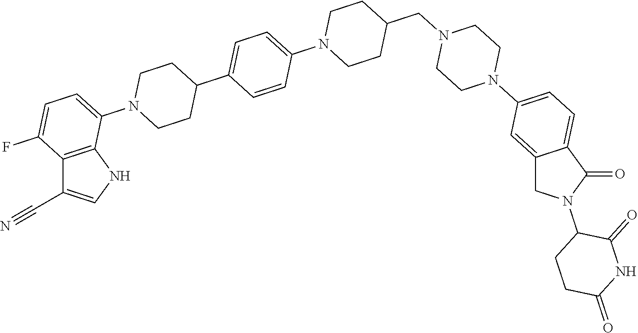 [SMILES: N#Cc1c[nH]c2c(N3CCC(c4ccc(N5CCC(CN6CCN(c7ccc8c(c7)CN(C7CCC(=O)NC7=O)C8=O)CC6)CC5)cc4)CC3)ccc(F)c12]

or a pharmaceutically acceptable salt thereof.

In one embodiment, this specification relates to a compound which is 7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile

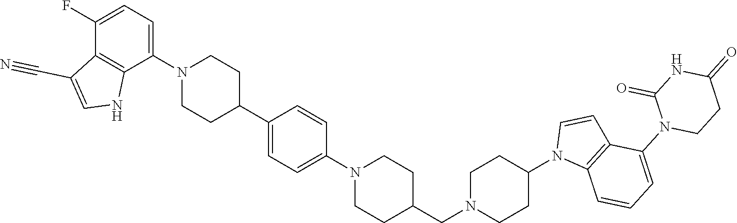 [SMILES: N#Cc1c[nH]c2c(N3CCC(c4ccc(N5CCC(CN6CCC(n7ccc8c(N9CCC(=O)NC9=O)cccc87)CC6)CC5)cc4)CC3)ccc(F)c12]

or a pharmaceutically acceptable salt thereof.

In one embodiment, this specification relates to a compound which is 4-Chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1H-indole-3-carbonitrile

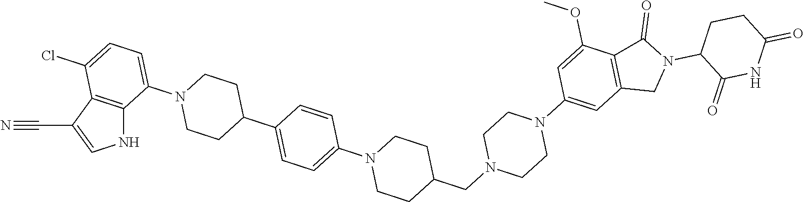 [SMILES: COc1cc(N2CCN(CC3CCN(c4ccc(C5CCN(c6ccc(Cl)c7c(C#N)c[nH]c67)CC5)cc4)CC3)CC2)cc2c1C(=O)N(C1CCC(=O)NC1=O)C2]

or a pharmaceutically acceptable salt thereof.

In one embodiment, this specification relates to a compound which is 7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-methyl-1H-indole-3-carbonitrile

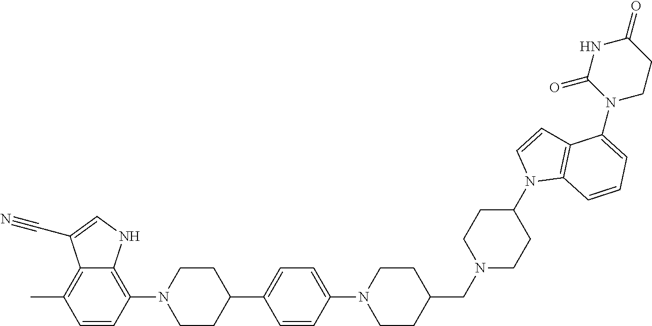 [SMILES: Cc1ccc(N2CCC(c3ccc(N4CCC(CN5CCC(n6ccc7c(N8CCC(=O)NC8=O)cccc76)CC5)CC4)cc3)CC2)c2[nH]cc(C#N)c12]

or a pharmaceutically acceptable salt thereof.

In another embodiment, this specification relates to a compound which is 4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile

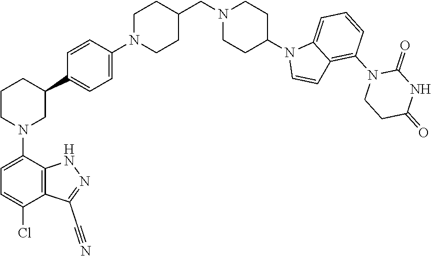 [SMILES: N#Cc1n[nH]c2c(N3CCC[C@@H](c4ccc(N5CCC(CN6CCC(n7ccc8c(N9CCC(=O)NC9=O)cccc87)CC6)CC5)cc4)C3)ccc(Cl)c12]

or a pharmaceutically acceptable salt thereof.

In another embodiment, this specification relates to a compound which is 4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indazole-3-carbonitrile

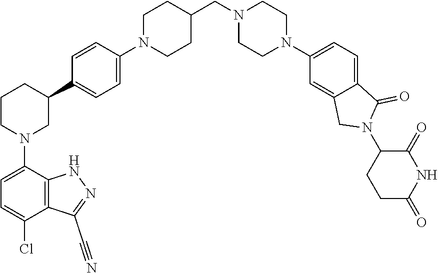 [SMILES: N#Cc1n[nH]c2c(N3CCC[C@@H](c4ccc(N5CCC(CN6CCN(c7ccc8c(c7)CN(C7CCC(=O)NC7=O)C8=O)CC6)CC5)cc4)C3)ccc(Cl)c12]

or a pharmaceutically acceptable salt thereof.

In another embodiment, this specification relates to a compound which is 4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile

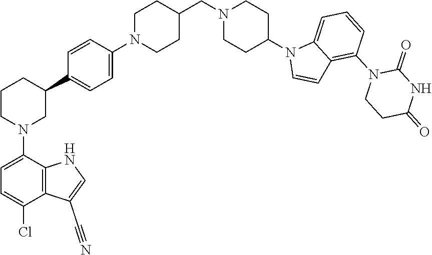 [SMILES: N#Cc1c[nH]c2c(N3CCC[C@@H](c4ccc(N5CCC(CN6CCC(n7ccc8c(N9CCC(=O)NC9=O)cccc87)CC6)CC5)cc4)C3)ccc(Cl)c12]

or a pharmaceutically acceptable salt thereof.

In another embodiment, this specification relates to a compound which is 4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile

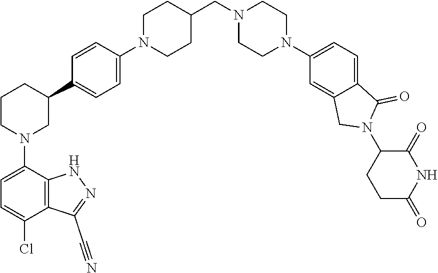 [SMILES: N#Cc1n[nH]c2c(N3CCC[C@@H](c4ccc(N5CCC(CN6CCN(c7ccc8c(c7)CN(C7CCC(=O)NC7=O)C8=O)CC6)CC5)cc4)C3)ccc(Cl)c12]

or a pharmaceutically acceptable salt thereof.

In another embodiment, this specification relates to a compound which is 4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indole-3-carbonitrile

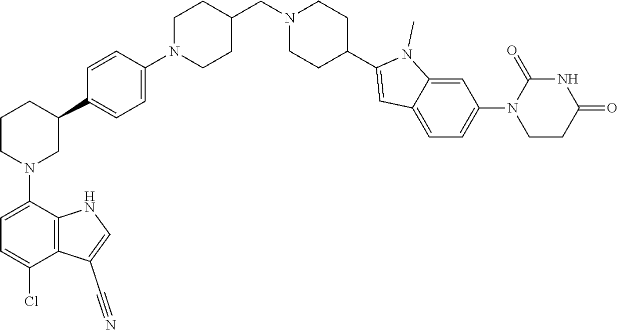 [SMILES: Cn1c(C2CCN(CC3CCN(c4ccc([C@@H]5CCCN(c6ccc(Cl)c7c(C#N)c[nH]c67)C5)cc4)CC3)CC2)cc2ccc(N3CCC(=O)NC3=O)cc21]

or a pharmaceutically acceptable salt thereof.

In another embodiment, this specification relates to a compound which is 4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile

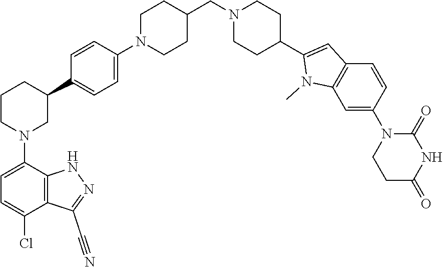 [SMILES: Cn1c(C2CCN(CC3CCN(c4ccc([C@@H]5CCCN(c6ccc(Cl)c7c(C#N)n[nH]c67)C5)cc4)CC3)CC2)cc2ccc(N3CCC(=O)NC3=O)cc21]

or a pharmaceutically acceptable salt thereof.

In another embodiment, this specification relates to a compound which is 7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indole-3-carbonitrile

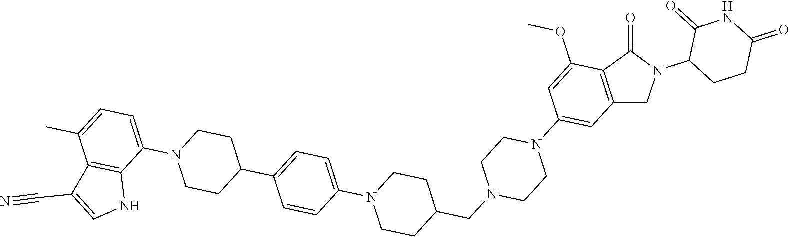 [SMILES: COc1cc(N2CCN(CC3CCN(c4ccc(C5CCN(c6ccc(C)c7c(C#N)c[nH]c67)CC5)cc4)CC3)CC2)cc2c1C(=O)N(C1CCC(=O)NC1=O)C2]

or a pharmaceutically acceptable salt thereof.

In another embodiment, this specification relates to a compound which is 7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indole-3-carbonitrile

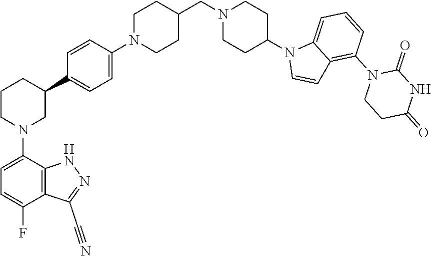 [SMILES: N#Cc1n[nH]c2c(N3CCC[C@@H](c4ccc(N5CCC(CN6CCC(n7ccc8c(N9CCC(=O)NC9=O)cccc87)CC6)CC5)cc4)C3)ccc(F)c12]

or a pharmaceutically acceptable salt thereof.

In another embodiment, this specification relates to a compound which is 7-{4-[4-(4-{4-[4-(2,4-Dioxo-1,3-diazinan-1-yl)-1H-indol-1-yl]piperidin-1-yl}butoxy)phenyl]piperidin-1-yl}-4-fluoro-1H-indole-3-carbonitrile

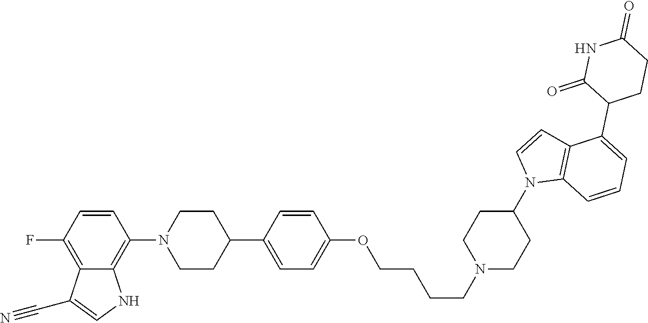 [SMILES: N#Cc1c[nH]c2c(N3CCC(c4ccc(OCCCCN5CCC(n6ccc7c(C8CCC(=O)NC8=O)cccc76)CC5)cc4)CC3)ccc(F)c12]

or a pharmaceutically acceptable salt thereof.

In another embodiment, this specification relates to a compound which is 4-Chloro-7-[(3S)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile

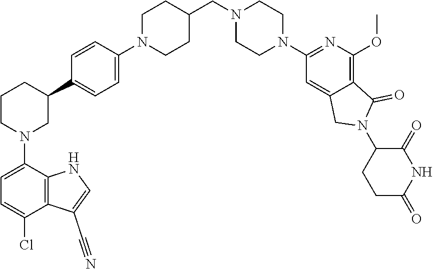 [SMILES: COc1nc(N2CCN(CC3CCN(c4ccc([C@@H]5CCCN(c6ccc(Cl)c7c(C#N)c[nH]c67)C5)cc4)CC3)CC2)cc2c1C(=O)N(C1CCC(=O)NC1=O)C2]

or a pharmaceutically acceptable salt thereof.

In another embodiment, this specification relates to a compound which is 4-Chloro-7-[(3S)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile

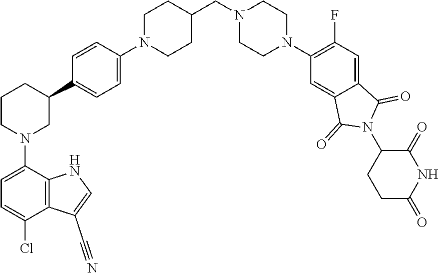 [SMILES: N#Cc1c[nH]c2c(N3CCC[C@@H](c4ccc(N5CCC(CN6CCN(c7cc8c(cc7F)C(=O)N(C7CCC(=O)NC7=O)C8=O)CC6)CC5)cc4)C3)ccc(Cl)c12]

or a pharmaceutically acceptable salt thereof.

In another embodiment, this specification relates to a compound which is 4-Chloro-7-[(3S)-3-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl]-1H-indole-3-carbonitrile

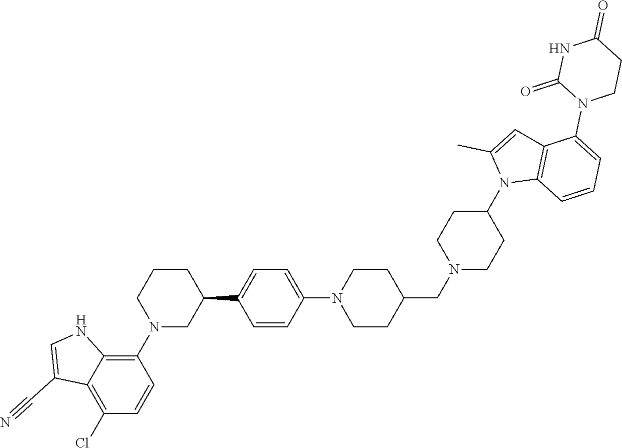 [SMILES: Cc1cc2c(N3CCC(=O)NC3=O)cccc2n1C1CCN(CC2CCN(c3ccc([C@@H]4CCCN(c5ccc(Cl)c6c(C#N)c[nH]c56)C4)cc3)CC2)CC1]

or a pharmaceutically acceptable salt thereof.

A further embodiment provides any of the embodiments, claims or aspects defined herein with the proviso that one or more specific Examples (for instance one Example, or two or three specific Examples) selected from the group consisting of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 153, 164, 165, 166, 167, 168, 169, 170, 171, 172, 173, 174, 175, 176, 177, 178, 179, 180 and 181 is individually disclaimed.

The compounds of Formula (I) and PROTAC compounds containing binding units of Formula (Ia) may have one or more chiral centres and it will be recognised that such compounds may be prepared, isolated and/or supplied with or without the presence of one or more of the other possible enantiomeric and/or diastereomeric isomers of said compounds or that such isomers may be provided in any relative proportions.

The preparation of enantioenriched/enantiopure and/or diastereoenriched/diastereopure compounds may be carried out by standard techniques of organic chemistry that are well known in the art, for example by synthesis from enantioenriched or enantiopure starting materials, and/or by use of an appropriately enantioenriched or enantiopure catalyst during synthesis, and/or by resolution of a racemic or partially enriched mixture of stereoisomers, for example via chiral chromatography.

Accordingly, in one embodiment there is provided a composition comprising a compound of Formula (I) [or PROTAC compound containing a unit of Formula (Ia)] or a pharmaceutically acceptable salt thereof, optionally together with one or more of the other stereoisomeric forms of the compound of Formula (I) [or PROTAC compound containing a unit of Formula (Ia)] or pharmaceutically acceptable salt thereof, wherein the compound of Formula (I) [or PROTAC compound containing a unit of Formula (Ia)] or pharmaceutically acceptable salt thereof is present within the composition with a diastereomeric excess (% de) of 90%.

In a further embodiment the % de in the above-mentioned composition is 95%.

In a further embodiment the % de in the above-mentioned composition is 98%.

In a further embodiment the % de in the above-mentioned composition is 99%.

In a further embodiment there is provided a composition comprising a compound of Formula (I) [or PROTAC compound containing a unit of Formula (Ia) as defined herein] or a pharmaceutically acceptable salt thereof, optionally together with one or more of the other stereoisomeric forms of the compound of Formula (I) [or PROTAC compound containing a unit of Formula (Ia) as defined herein] or pharmaceutically acceptable salt thereof, wherein the compound of Formula (I) [or PROTAC compound containing a unit of Formula (Ia) as defined herein] or pharmaceutically acceptable salt thereof is present within the composition with an enantiomeric excess (% ee) of 90%.

In a further embodiment the % ee in the above-mentioned composition is 95%.

In a further embodiment the % ee in the above-mentioned composition is 98%.

In a further embodiment the % ee in the above-mentioned composition is 99%.

In a further embodiment there is provided a composition comprising a compound of Formula (I) [or PROTAC compound containing a unit of Formula (Ia) as defined herein] or a pharmaceutically acceptable salt thereof, optionally together with one or more of the other stereoisomeric forms of the compound of Formula (I) [or PROTAC compound containing a unit of Formula (Ia) as defined herein], or pharmaceutically acceptable salt thereof, wherein the compound of Formula (I) [or PROTAC compound containing a unit of Formula (Ia) as defined herein], or pharmaceutically acceptable salt thereof is present within the composition with an enantiomeric excess (% ee) of 90% and a diastereomeric excess (% de) of 90%.

In further embodiments of the above-mentioned composition the % ee and % de may take any combination of values as listed below:
The % ee is ≤5% and the % de is ≤80%.
The % ee is ≤5% and the % de is ≤90%.
The % ee is ≤5% and the % de is ≤95%.
The % ee is ≤5% and the % de is ≤98%.
The % ee is ≤95% and the % de is ≤95%.
The % ee is ≤98% and the % de is ≤98%.
The % ee is ≤99% and the % de is ≤99%.

In a further embodiment there is provided a pharmaceutical composition which comprises a compound of the Formula (I) [or PROTAC compound containing a unit of Formula (Ia) as defined herein], or a pharmaceutically acceptable salt thereof, in association with a pharmaceutically acceptable excipient.

In one embodiment there is provided a pharmaceutical composition which comprises a compound of the Formula (I) [or PROTAC compound containing a unit of Formula (Ia) as defined herein], or a pharmaceutically acceptable salt thereof, in association with a pharmaceutically acceptable excipient, optionally further comprising one or more of the other stereoisomeric forms of the compound of Formula (I) [or PROTAC compound containing a unit of Formula (Ia) as defined herein], or pharmaceutically acceptable salt thereof, wherein the compound of Formula (I) [or PROTAC compound containing a unit of Formula (Ia) as defined herein], or pharmaceutically acceptable salt thereof is present within the composition with an enantiomeric excess (% ee) of ≥90%.

In a further embodiment the % ee in the above-mentioned composition is ≥95%.

In a further embodiment the % ee in the above-mentioned composition is ≥98%.

In a further embodiment the % ee in the above-mentioned composition is ≥99%.

In one embodiment there is provided a pharmaceutical composition which comprises a compound of the Formula (I) [or PROTAC compound containing a unit of Formula (Ia) as defined herein], or a pharmaceutically acceptable salt thereof, in association with a pharmaceutically acceptable excipient, optionally further comprising one or more of the other stereoisomeric forms of the compound of Formula (I) [or PROTAC compound containing a unit of Formula (Ia) as defined herein], or pharmaceutically acceptable salt thereof, wherein the compound of Formula (I) [or PROTAC compound containing a unit of Formula (Ia) as defined herein], or pharmaceutically acceptable salt thereof is present within the composition with a diastereomeric excess (% de) of ≥90%.

In a further embodiment the % de in the above-mentioned composition is ≥95%.

In a further embodiment the % de in the above-mentioned composition is ≥98%.

In a further embodiment the % de in the above-mentioned composition is ≥99%.

In one embodiment there is provided a pharmaceutical composition which comprises a compound of the Formula (I) [or PROTAC compound containing a unit of Formula (Ia) as defined herein], or a pharmaceutically acceptable salt thereof, in association with a pharmaceutically acceptable excipient, optionally further comprising one or more of the other stereoisomeric forms of the compound of Formula (I) [or PROTAC compound containing a unit of Formula (Ia) as defined herein], or pharmaceutically acceptable salt thereof, wherein the compound of Formula (I) [or PROTAC compound containing a unit of Formula (Ia) as defined herein], or pharmaceutically acceptable salt thereof is present within the composition with an enantiomeric excess (% ee) of ≥90% and a diastereomeric excess (% de) of ≥90%.

In further embodiments of the above-mentioned pharmaceutical composition the % ee and % de may take any combination of values as listed below:
The % ee is ≥95% and the % de is ≥95%.
The % ee is ≥98% and the % de is ≥98%.
The % ee is ≥99% and the % de is ≥99%.

The compounds of Formula (I) [or PROTAC compound containing a unit of Formula (Ia) as defined herein], and pharmaceutically acceptable salts thereof may be prepared, used or supplied in amorphous form, crystalline form, or semicrystalline form and any given compound of Formula (I) [or PROTAC compound containing a unit of Formula (Ia) as defined herein], or pharmaceutically acceptable salt thereof may be capable of being formed into more than one crystalline/polymorphic form, including hydrated (e.g. hemi-hydrate, a mono-hydrate, a di-hydrate, a tri-hydrate or other stoichiometry of hydrate) and/or solvated forms. It is to be understood that the present specification encompasses any and all such solid forms of the compound of Formula (I) [or PROTAC compound containing a unit of Formula (Ia) as defined herein], and pharmaceutically acceptable salts thereof.

In further embodiments there is provided a compound of Formula (I) [or PROTAC compound containing a unit of Formula (Ia) as defined herein] which is obtainable by the methods described in the ‘Examples’ section hereinafter.

### Intermediate Compounds

As demonstrated in the experimental section hereinafter, the compounds of Formula (I) or PROTACs of Formula (Ia) may be prepared, for example, by the following methods.

A compound of Formula (I) [or salt thereof] or a PROTAC compound of Formula (Ia) [or a salt thereof] may be prepared from a compound of Formula (II):

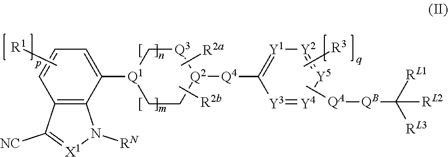

or a salt thereof, where:
Q^{A} is -G-Q^{H}- or -G-(C₁₋₅alkylene)-;
G is a direct bond, —CH₂—, —C(═O)—, —O—, or —N(R^{G})— where R^{G} is H or C₁₋₃alkyl;
Q^{H} is a 4 to 12-membered nitrogen-containing saturated or partially unsaturated heterocyclic group;
Q^{D} is a direct bond or C₁₋₂alkylene optionally substituted by one or more F;
R^{L1} and R^{L2} together form “═O”, R^{L3} is H;
where when Q^{D} is a direct bond and Q^{A} is -G-Q^{H}-, the value of Q^{H} is selected so that Q^{D} connects to a C atom of Q^{H}; and
R¹, p, X¹, R^{N}, n, M, R^{2a}, R^{2b}, Q¹, Q², Q³, Q⁴, Y¹, Y², Y³, Y⁴, Y⁵, R³ and q may take any of the values disclosed herein for each of those groups/variables respectively.

In one embodiment Q^{H} is a 4-12-membered nitrogen-containing saturated heterocyclic group.

Accordingly such compound of Formula (II) as described above is an aldehyde which may be reacted with a suitable molecule containing an amine group (for example a secondary amine group) to form the compound of Formula (I) or PROTAC compound of Formula (Ia)—either directly, or after one or more additional reaction steps. Such reaction of said aldehyde with said amine can be carried out under reductive amination conditions, using for example NaBH(OAc)₃ or another reductive amination protocol known to the skilled person.

Such aldehyde may in turn be prepared from the corresponding acetal, for example a compound of Formula (II), or salt thereof, as defined above, except that: R^{L1} and R^{L2} are each independently C₁₋₆alkoxy (for example C₁₋₃alkoxy) or R^{L1} & R^{L2} together form —O—(CH₂)ₖ—O— where k is 2 or 3. Such acetal may be converted to the corresponding aldehyde under acidic conditions, for example using formic acid under conditions well known to the skilled person.

Alternatively, as shown in the experimental section hereinafter, the above-mentioned aldehyde may be prepared by oxidation of the corresponding primary alcohol, i.e. a compound of Formula (II), or salt thereof, as defined above except that R^{L1} is OH and R^{L2} is H. Such oxidation may be carried out using mild oxidising conditions, for example Dess-Martin periodinane or some other mild oxidation protocol known to the skilled person.

As shown in the experimental section hereinafter, a compound of Formula (I) [or salt thereof] or a PROTAC compound of Formula (Ia) [or a salt thereof] may be prepared from a compound of Formula (II) or a salt thereof, as described above except that R^{L1} is a leaving group and R^{L2} is H. Accordingly, such compound of Formula (II) is an electrophile that may be reacted with a molecule containing an amine group (for example a secondary amine group) via an alkylation reaction to form the compound of Formula (I) or PROTAC compound of Formula (Ia)—either directly, or after one or more additional reaction steps. Suitable leaving groups for alkylation reactions are well known to the skilled person and include Cl, Br, I, trifluoromethanesulfonate, mesylate and tosylate. Alkylation reaction conditions are well known to the skilled person and generally involve a non-nucleophilic base (e.g. DIPEA) and a polar aprotic solvent (e.g. MeCN).

As demonstrated in the experimental section hereinafter, in cases where the leaving group is not I, a metal iodide salt may be used in the reaction mixture to form the corresponding iodide in-situ (i.e. where R^{L1} is I) to facilitate the overall alkylation process. As shown in the experimental section hereinafter, a compound of Formula (II) where R^{L1} is a bromo leaving group may be prepared from the corresponding primary alcohol (i.e. as already described above where R^{L1} is OH, and R^{L2} & R^{L3} are both H.

In turn, the above-mentioned primary alcohol compound of Formula (II) may be prepared by the reduction of the corresponding ester compound, i.e. a compound of Formula (II), or salt thereof, as defined above, except that: R^{L1} and R^{L2} together form “═O” and R^{L3} is C₁₋₆alkoxy (for example C₁₋₃alkoxy). Such reduction may be carried out using strong reduction conditions, for example using DIBAL or other stronger reducing conditions which are well known to the skilled person.

Alternatively, as demonstrated in the experimental section hereinafter, it may be convenient to form the above-mentioned primary alcohol compound of Formula (II) via deprotection of a protected form of the alcohol. For example, deprotection of a compound where there alcohol is protected by a silicon-based protecting group, using a source of fluoride in order to achieve the deprotection, for example TBAF or other deprotection methods well-known to the skilled person.

Therefore, as described above, various compounds of Formula (II), and salts thereof, may be useful as intermediates in the synthesis of the compounds of Formula (I) or PROTAC compounds of Formula (Ia), and accordingly such intermediate compounds provide a further aspect of the specification.

Therefore, in a further aspect there is provided a compound of Formula (II), as shown above, or a salt thereof, wherein:
Q^{A} is -G-Q^{H}- or -G-(C₁₋₅alkylene)-;
G is a direct bond, —CH₂—, —C(═O)—, —O—, or —N(R^{G})— where R^{G} is H or C₁₋₃alkyl;
Q^{H} is a 4-12-membered nitrogen-containing saturated or partially unsaturated heterocyclic group;
Q^{D} is a direct bond or C₁₋₂alkylene optionally substituted by one or more F (for example 1 or 2 F); and
   (i) R^{L1} & R^{L2} together form “═O” and R^{L3} is H or C₁₋₆alkoxy (for example OMe);
   (ii) R^{L1} & R^{L2} are each independently C₁₋₆alkoxy (for example OMe), and R^{L3} is H;
   (iii) R^{L1} & R^{L2} together form —O—(CH₂)ₖ—O— where k is 2 or 3, and R^{L3} is H; or
   (iv) R^{L1} is OH, OPG¹ (where PG¹ is a protecting group), or LG¹ (where LG¹ is a leaving group), and R^{L2} & R^{L3} are both H;
where when Q^{D} is a direct bond and Q^{A} is -G-Q^{H}-, the value of Q^{H} is selected so that Q^{D} connects to a C atom of Q^{H}.
and R¹, p, X¹, X², X³, X⁴, n, m, Q, R^{2a}, R^{2b}, Y¹, Y², Y³, Y⁴, R³ and q may take any of the values disclosed herein for each of those groups/variables respectively.

In one embodiment Q^{H} is a 4-12-membered nitrogen-containing saturated heterocyclic group.

The skilled person is aware of suitable protecting groups for alcohol groups and therefore is aware of suitable values of PG¹. For example PG¹ is an alcohol protecting group.

In one embodiment PG¹ is a silicon-based alcohol protecting group.

In one embodiment PG¹ is Si(R^{Si})₃ where each R^{Si} is independently a C₁₋₆hydrocarbyl group.

In one embodiment PG¹ is tert-butyldimethylsilyl or tert-butyldiphenylsilyl.

In one embodiment the LG¹ is selected from Cl, Br, I, trifluoromethanesulfonate and C₁₋₇hydrocarbylsulfonate (for example mesylate orp-toluenesulfonate).

In one embodiment LG¹ is Br or I.

In one embodiment LG¹ is Br.

In one embodiment LG¹ is Cl.

In one embodiment LG¹ is I.

In one embodiment LG¹ is trifluoromethanesulfonate.

In one embodiment LG¹ is C₁₋₇hydrocarbylsulfonate.

In one embodiment LG¹ is mesylate.

In one embodiment LG¹ is p-toluenesulfonate.

In one embodiment Q^{D} is a direct bond, —CH₂—, —CH₂CH₂— or —CF₂CH₂—.

In one embodiment Q¹ is a direct bond.

In one embodiment Q^{D} is CH₂.

In one embodiment Q^{D} is C₁₋₂alkylene optionally substituted by 1 or 2 F.

In one embodiment Q^{D} is C₁₋₂alkylene.

In one embodiment Q^{D} is —CH₂CH₂—.

In one embodiment Q^{D} is —CF₂CH₂—.

In further embodiments of this specification Formula (II) may be Formula (IIa):

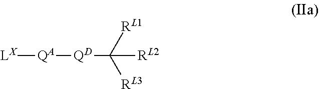

where L^{X} may be any of the groups (1) to (43) listed hereinabove in connection with Formula (Ia); and where Q^{A}, Q^{D}, R^{L1}, R^{L2} and R^{L3} may take any of the values disclosed herein for each of said groups respectively.

As demonstrated in the experimental section hereinafter, certain compounds of Formula (I) and certain PROTACS of Formula (Ia) and certain intermediate compounds of Formula (II) may be prepared using a compound of Formula (III):

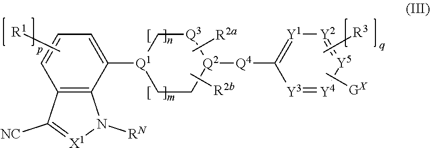

or a salt thereof, where G^{X} is OH, where the oxygen atom of said OH is alkylated by a suitable molecule to form certain compounds of Formula (I) or PROTACs of Formula (Ia)—either directly, or after one or more further reaction steps (for example via Formula (II) as described hereinabove). Such alkylation reactions may be carried out under conditions well known to the skilled person, for example using a primary alkyl bromide (or using some other leaving group in place of Br) using a non-nucleophilic base such as a metal carbonate (e.g. K₂CO₃) in a polar aprotic solvent such as MeCN, optionally in the presence of a metal iodide salt such as KI.

Alternatively, as shown in the experimental section hereinafter, a compound of Formula (III) where G^{X} is —NH(R^{G}) [where R^{G} is H or C₁₋₃alkyl (for example Me)] may be used as an intermediate to prepare compounds of Formula (I) or PROTACs of Formula (Ia) via reductive amination chemistry with an appropriate aldehyde-containing compound.

Alternatively, as shown in the experimental section hereinafter, a compound of Formula (III) where G^{X} is bromo may be coupled with a secondary amine compound to give certain compounds of Formula (I) or PROTACs of Formula (Ia)—either directly, or after one or more further reaction steps (for example via Formula (II) as described hereinabove). Similarly, compounds of Formula (III) where G^{X} is chloro or trifluoromethanesulfonate may also be used in a coupling reaction with the relevant secondary amine to give certain compounds of Formula (I) or PROTACs of Formula (Ia). Such coupling may be carried out under palladium-based coupling conditions (e.g. using ‘Ruphos Pd G3’ and ‘Ruphos’) in the presence of a base and an anhydrous solvent such as 1,4-dioxane under an inert atmosphere, or by heating with CuI in the presence of a base such as K₃PO₄ in a polar solvent such as DMSO. Alternatively, also as shown in the experimental section herein, a compound of Formula (III) where G^{X} is bromo may be coupled with a suitable alcohol to form certain compounds of Formula (I) or PROTACs of Formula (Ia)—either directly, or after one or more further reaction steps (for example via Formula (II) as described hereinabove). Such coupling may be carried out using a palladium-based reagent such as ‘Rockphos Pd G3’ in the presence of a base such as Cs₂CO₃ in a solvent such as toluene.

Accordingly, compounds of Formula (III), and salts thereof, may be useful as intermediates in the synthesis of certain compounds of Formula (I) or PROTAC compounds of Formula (Ia) or compounds of Formula (II), and accordingly such intermediate compounds provide a further aspect of the specification.

Therefore, in a further aspect there is provided a compound of Formula (III), as shown above, or a salt thereof, wherein:
G^{X} is OH, Cl, Br, triflouoromethanesulfonate or —NH(R^{G}) where R^{G} is H or C₁₋₃alkyl; and
R¹, p, X¹, R^{N}, n, m, R^{2a}, R^{2b}, Q¹, Q², Q³, Q⁴, Y¹, Y², Y³, Y⁴, Y⁵, R³ and q may take any of the values disclosed herein for each of those groups/variables respectively.

G^{X} is OH, Cl, Br, triflouoromethanesulfonate or —NH(R^{G}) where R^{G} is C₁₋₃alkyl (for example Me).

In one embodiment G^{X} is OH, Br, trifluoromethanesulfonate or —NH(R^{G}) where R^{G} is C₁₋₃alkyl (for example Me).

In one embodiment G^{X} is OH, Br or —NH(R^{G}) where R^{G} is C₁₋₃alkyl (for example Me).

In one embodiment G^{X} is OH or Br.

In one embodiment G^{X} is OH.

In one embodiment G^{X} is Br.

In one embodiment G^{X} is Cl.

In one embodiment G^{X} is trifluoromethanesulfonate.

In one embodiment G^{X} is —NH(R^{G}) where R^{G} is H or C₁₋₃alkyl.

In one embodiment G^{X} is —NH(R^{G}) where R^{G} is C₁₋₃alkyl.

In one embodiment G^{X} is —NH(Me).

In further embodiments of this specification, Formula (III) may be L^{X}-G^{X} wherein L^{X} may be any of the groups (1) to (43) listed hereinabove in connection with Formula (Ia); and where G^{X} may take any value(s) disclosed herein for G^{X}.

As demonstrated in the experimental section hereinafter, certain compounds of Formula (I) and PROTACs of Formula (Ia) may be prepared using an intermediate compound of Formula (IV):

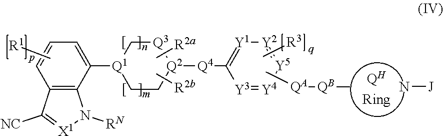

or a salt thereof, as further defined below. For example, a compound of Formula (IV), or salt thereof, where J is H (i.e. a secondary amine compound) may be coupled to a further chemical fragment, using chemistry well known to the skilled person and exemplified in the experimental section hereinafter, to provide a compound of Formula (I) or PROTAC of Formula (Ia), either directly, or after one or more further reaction steps.

In turn, such a compound of Formula (IV) where J is H may conveniently be prepared via deprotection of an N-protected form of the aforementioned amine compound. Accordingly compounds of Formula (IV) where J is H may be conveniently be prepared using a compound of Formula (IV) where J is PG² where PG² is a nitrogen protecting group (for example a C₁₋₆alkoxycarbonyl group such as tert-butoxycarbonyl). Therefore, compounds of Formula (IV) where J is PG² are useful intermediates in the preparation of the compound of Formula (I) and PROTAC of Formula (Ia) and provide a further aspect of the specification.

Therefore in a further aspect of the specification there is provided a compound of Formula (IV) as depicted above, or a salt thereof, wherein:
J is H or PG² where PG² is a nitrogen protecting group (for example a tert-butoxycarbonyl group);
Q^{A} is -G-Q^{H}- or -G-(C₁₋₅alkylene)-;
G is a direct bond, —CH₂—, —C(═O)—, —O—, or —N(R^{G})— where R^{G} is H or C₁₋₃alkyl;
Q^{B} is a direct bbond, -Q^{B1}-Q^{B2}-Q^{B3}- or C₁₋₃alkylene optionally substituted by one or more F (e.g. 1 or 2);
   where:
   Q^{B1} & Q^{B3} each independently represent a direct bond or C₁₋₂alkylene;
   Q^{B2} is Q^{H}, —O—CH₂CH₂—O—, —O— or —N(R^{J})— where R^{J} is H or C₁₋₃alkyl;
and each Q^{H} (including the “Q^{H} Ring” attached to J) is independently a 4-12-membered nitrogen-containing saturated or partially unsaturated heterocyclic group;
where the values of Q^{A}, Q^{B} and Q^{H} Ring are selected so that Formula (TV) does not contain any N—N or N—O bonds;
and where R¹, p, X¹, R^{N}, n, m, R^{2a}, R^{2b}, Q¹, Q², Q³, Q⁴, Y¹, Y², Y³, Y⁴, Y⁵, R³ and q may take any of the values defined herein for each of these groups/variables respectively.

In one embodiment each Q^{H} (including the “Q^{H} Ring” attached to J) is independently a 4-12-membered nitrogen-containing saturated heterocyclic group.

In one embodiment the Q^{H} Ring is piperidin-1,4-diyl or piperazin-1,4-diyl.

In one embodiment J is H.

In one embodiment J is PG².

In one embodiment PG² is C₁₋₆alkoxycarbonyl.

In one embodiment PG² is tert-butoxycarbonyl.

In one embodiment where PG² is C₁₋₆alkoxycarbonyl (e.g. tert-butoxycarbonyl) the ‘and salts thereof’ element of the claim is excluded.

In further embodiments the compound of Formula (IV) may take any combination of alternative values mentioned in relation to Q^{A}, Q^{B} and Q^{H} in any other context, embodiment, aspect or claim found herein.

In further embodiments of this specification Formula (IV) may be Formula (IVa):

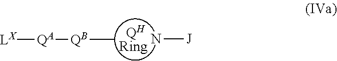

where L^{X} may be any of the groups (1) to (43) listed hereinabove in connection with Formula (Ia); and where Q^{A}, Q^{B}, Q^{H} and J may take any of the values disclosed herein for each of said groups respectively.

As also demonstrated in the experimental section hereinafter, certain compounds of Formula (I) and PROTACs of Formula (Ia) may be prepared using an intermediate compound of Formula (V):

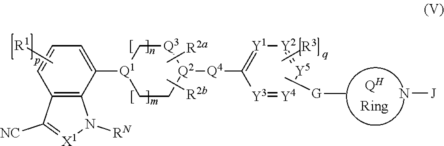

or a salt thereof, wherein:
X^{X} is N substituted by J where J is H;
G is a direct bond, —CH₂—, —C(═O)—, —O—, or —N(R^{G})— where R^{G} is H or C₁₋₃alkyl;
Q^{H} Ring is a 4-12-membered nitrogen-containing saturated or partially unsaturated heterocyclic group;
where the values of G and Q^{H} Ring are selected so that Formula (V) does not contain any N—N or N—O bonds; and where R¹, p, X¹, R^{N}, n, m, R^{2a}, R^{2b}, Q¹, Q², Q³, Q⁴, Y¹, Y², Y³, Y⁴, Y⁵, R³ and q may take any of the values defined herein for each of these groups/variables respectively.

Such a compound of Formula (V) may be converted to a compound of Formula (I) or PROTAC of Formula (Ia) via reductive amination or alkylation or other coupling chemistry known to the skilled person—to provide the compound of Formula (I) or PROTAC of Formula (Ia) either directly, or after one or more additional steps.

In turn, as shown in the experimental section hereinafter, such a compound of Formula (V), or a salt thereof, may be conveniently prepared by deprotection of the corresponding N-protected compound. Such N-protected form may be a BOC-protected form (i.e. tert-butoxycarbonyl) or may use another N-protecting group known to the skilled person. Accordingly, such N-protected compounds are also useful intermediates in the preparation of the compounds of Formula (I) and PROTACs of Formula (Ia) and provide a further aspect of the specification.

Accordingly one aspect of the specification provides a compound of Formula (V), or a salt thereof, as described above except that X^{X} is N substituted by J where J is PG³ and PG³ is a protecting group. In one embodiment PG³ is C₁₋₆alkoxycarbonyl. In one embodiment PG³ is tert-butoxycarbonyl. In one embodiment where PG³ is C₁₋₆alkoxycarbonyl (e.g. tert-butoxycarbonyl) the ‘and salts thereof’ element of the claim is excluded.

Other compounds of Formula (I) and PROTACs of Formula (Ia)—and salts thereof—may be prepared from an intermediate compound of Formula (V), or a salt thereof, as described above except that X^{X} is C═O. Such an intermediate may be converted to a compound of Formula (I) or PROTAC of Formula (Ia) by reductive amination chemistry using an appropriate amine-containing compound, using reductive amination conditions that well-known to the skilled person - either directly or via one or more additional synthetic steps.

In turn, as demonstrated in the experimental section hereinafter, such compounds of Formula (V) where X^{X} is C═O may be conveniently prepared from the corresponding compound where the ketone is protected/masked as a ketal. Accordingly such ketal compounds, and salts thereof are useful intermediates in the preparation of a compound of Formula (I) or a PROTAC of Formula (Ia) and provide a further aspect of the specification.

Accordingly such ketal compound may be represented as a compound of Formula (V), or a salt thereof, as described above except that X^{X} is C substituted by R^{U1} and R^{U2}; where R^{U1} and R^{U2} are each C₁₋₆alkoxy; or R^{U1} and R^{U2} together represent —O—(CH₂)ᵤ—O— where u is 2 or 3.

Therefore, in a further aspect of the specification there is provided compound of Formula (V), as depicted above, or a salt thereof, wherein:
X^{X} is selected from:
   (i) N substituted by J where J is H or PG³ where PG³ is a protecting group; and
   (ii) C substituted by oxo, or by R^{U1} and R^{U2}; where R^{U1} and R^{U2} are each C₁₋₆alkoxy; or R^{U1} and R^{U2} together represent —O—(CH₂)ᵤ—O— where u is 2 or 3;
G is a direct bond, —CH₂—, —C(═O)—, —O—, or —N(R^{G})— where R^{G} is H or C₁₋₃alkyl;
Q^{H} Ring is a 4-12-membered nitrogen-containing saturated or partially unsaturated heterocyclic group; and the values of G and Q^{H} Ring are selected so that Formula (V) does not contain any N—N or N—O bonds; and where R¹, p, X¹, R^{N}, n, m, R^{2a}, R^{2b}, Q¹, Q², Q³, Q⁴, Y¹, Y², Y³, Y⁴, Y⁵, R³ and q may take any of the values defined herein for each of these groups/variables respectively.

In one embodiment J is H.

In one embodiment J is PG³.

In one embodiment PG³ is C₁₋₆alkoxycarbonyl.

In one embodiment PG³ is tert-butoxycarbonyl.

In one embodiment X^{X} is C═O.

In one embodiment X^{X} is C substituted by R^{U1} and R^{U2}; where R^{U1} and R^{U2} are each C₁₋₆alkoxy.

In one embodiment X^{X} is C substituted by R^{U1} and R^{U2}; where R^{U1} and R^{U2} together represent —O—(CH₂)ᵤ—O— where u is 2 or 3 (for example u=2).

In one embodiment G is a direct bond or —O—.

In one embodiment Q^{H} Ring is a 4-12-membered nitrogen-containing saturated heterocyclic group.

The values of Q^{H} Ring may take any of the values mentioned herein for Q^{H}.

In one embodiment Q^{H} Ring is a piperidine ring, a piperazine ring, a 9-azaspiro[5.5]undecane ring or a 3,9-diazaspiro[5.5]undecane ring.

In further embodiments of this specification Formula (V) may be Formula (Va):

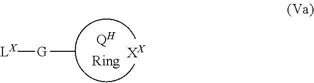

where L^{X} may be any of the groups (1) to (43) listed hereinabove in connection with Formula (Ia); and where G, Q^{H} Ring and X^{X} may take any of the values disclosed herein for each of said groups respectively.

In addition to the methods described above, the compounds of Formulae (I), (II), (III), (IV) & (V) and PROTAC compounds including Formula (Ia) may be prepared according to the general procedures and chemical transformations demonstrated in the experimental section hereinafter and using standard procedures and knowledge known to the skilled chemist.

In further embodiments of this specification there is/are provided compound(s), or a salt thereof, wherein said compound(s) is/are selected from one or more of the “Intermediates” listed hereinafter in the experimental section.

It is to be understood that the compound of an Intermediate listed hereinafter relates to the title chemical name listed in the experimental section, and is not limited in any way by the method of preparation nor whether a given intermediate compound was isolated in the form of a salt rather than as a neutral molecule.

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, in association with a pharmaceutically acceptable excipient.

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of cancer.

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of a solid tumour.

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of an AR-sensitive tumour type.

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of tumour types that harbour one or more mutated forms of the androgen receptor.

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of prostate cancer (for example CRPC, for example metastatic CRPC).

According to a further aspect of the specification there is provided a pharmaceutical composition, which comprises a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of AR-mutated cancer.

In any embodiment, aspect or claim herein that mentions “cancer” without further specificity, further embodiments, aspects or claims may be provided where said cancer is (or includes) AR+ breast cancer.

The compositions may be in a form suitable for oral use (for example as tablets, lozenges, hard or soft capsules, aqueous or oily suspensions, emulsions, dispersible powders or granules, syrups or elixirs) or for parenteral administration (for example as a sterile aqueous or oily solution for intravenous, subcutaneous or intramuscular dosing). The compositions may be obtained by conventional procedures using conventional pharmaceutical excipients that are well known in the art. Thus, compositions intended for oral use may contain, for example, one or more colouring, sweetening, flavouring and/or preservative agents.

For further information on formulations the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host being treated and the particular route of administration.

The size of the dose for therapeutic purposes of compounds of the present specification will naturally vary according to the nature and severity of the disease state, the age and sex of the animal or patient and the route of administration, according to well-known principles of medicine.

As explained hereinabove, the compounds of the present specification may be of value as anti-tumour agents, in particular as selective inhibitors of the proliferation, survival, motility, dissemination and invasiveness of mammalian cancer cells leading to inhibition of tumour growth and survival and to inhibition of metastatic tumour growth. Particularly, the compounds of the present specification may be of value as anti-proliferative and anti-invasive agents in the containment and/or treatment of solid tumour disease.

Accordingly, the compounds of the present specification may be useful in the prevention or treatment of those tumours which are sensitive to degradation of the androgen receptor and that are involved in the signal transduction steps which lead to the proliferation and survival of tumour cells and the migratory ability and invasiveness of metastasising tumour cells. Further, the compounds of the present specification may be useful in the prevention or treatment of those tumours which are treatable by degradation of androgen receptors, i.e. the compounds may be used to produce an androgen receptor degradation effect in a warm-blooded animal in need of such treatment.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use as a medicament.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in therapy.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in a method of treatment of the human or animal body by therapy.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein for use in the production of an anti-proliferative effect (for example, in a warm-blooded animal such as man).

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for the production of an anti-proliferative effect (for example, in a warm-blooded animal such as man).

According to a further aspect of the specification, there is provided a method for producing an anti-proliferative effect in a warm-blooded animal, such as man, in need of such effect, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use as an anti-invasive agent in the containment and/or treatment of solid tumour disease (for example: in a warm-blooded animal such as man).

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for use as an anti-invasive agent in the containment and/or treatment of solid tumour disease (for example: in a warm-blooded animal such as man).

According to a further aspect of the specification, there is provided a method for producing an anti-invasive effect by the containment and/or treatment of solid tumour disease, in a warm-blooded animal, such as man, in need of such effect, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the prevention or treatment of cancer (for example: in a warm-blooded animal such as man).

According to a further aspect of the specification there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for the prevention or treatment of cancer (for example: in a warm-blooded animal such as man).

According to a further aspect of the specification there is provided a method for the prevention or treatment of cancer in a warm-blooded animal, such as man, in need of such treatment, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the prevention or treatment of solid tumour(s) (for example, in a warm-blooded animal such as man).

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for the prevention or treatment of solid tumour(s) (for example, in a warm-blooded animal such as man).

According to a further aspect of the specification, there is provided a method for the prevention or treatment of solid tumour(s) in a warm-blooded animal, such as man, in need of such treatment, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the prevention or treatment of tumour types that are sensitive to degradation of androgen receptors.

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for the prevention or treatment of those tumour types that are sensitive to degradation of androgen receptors.

According to a further aspect of the specification, there is provided a method for the prevention or treatment of those tumour types that are sensitive to degradation of androgen receptors in a warm-blooded animal, such as man, in need of such treatment, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

As explained hereinabove, tumour types that are sensitive to degradation of androgen receptors include prostate cancer (for example castrate-resistant prostate cancer (CRPC), for example metastatic CRPC).

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in providing a degrading effect on androgen receptors (for example in a warm-blooded animal such as man).

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for providing a degrading effect on androgen receptors (for example in a warm-blooded animal such as man).

According to a further aspect of the specification, there is provided a method for providing a degrading effect on androgen receptors in a warm-blooded animal, such as man, in need of such effect, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in providing a selective degrading effect on androgen receptors (for example in a warm-blooded animal such as man).

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for providing a selective degrading effect on androgen receptors (for example in a warm-blooded animal such as man).

According to a further aspect of the specification, there is provided a method for providing a selective degrading effect on androgen receptors in a warm-blooded animal, such as man, in need of such effect, which comprises administering an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of tumour types that harbour androgen receptor mutations.

According to a further aspect of the specification, there is provided the use of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for the prevention or treatment of those tumour types that harbour androgen receptor mutations.

According to a further aspect of the specification, there is provided a method for the prevention or treatment of those tumour types that harbour androgen receptor mutations in a warm-blooded animal, such as man, in need of such prevention or treatment, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

As mentioned above, tumour types known to harbour androgen receptor mutations include prostate tumours and therefore prostate cancer, castrate-resistant prostate cancer (CRPC), and metastatic (CRPC).

According to a further aspect of the specification, there is provided a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for use in the treatment of prostate cancer (for example castrate-resistant prostate cancer (CRPC), for example metastatic CRPC).

According to a further aspect of the specification, there is provided the use of a compound of the Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein, for the manufacture of a medicament for the treatment of prostate cancer (for example castrate-resistant prostate cancer (CRPC), for example metastatic CRPC).

According to a further aspect of the specification, there is provided a method for treating prostate cancer (for example castrate-resistant prostate cancer (CRPC), for example metastatic CRPC) in a warm-blooded animal, such as man, in need of such treatment, which comprises administering to said animal an effective amount of a compound of Formula (I), [or a PROTAC compound containing an E3 ubiquitin ligase cereblon binder unit and (e.g. linked to) an AR binding unit of Formula (Ia)] or a pharmaceutically acceptable salt thereof, as defined herein.

In one embodiment where cancer is mentioned herein, said cancer is prostate cancer.

In one embodiment where cancer is mentioned herein, said cancer is CRPC.

In one embodiment where cancer is mentioned herein, said cancer is metastatic CRPC.

### GENERAL EXPERIMENTAL CONDITIONS AND ABBREVIATIONS

The following abbreviations are used: AcOH=Acetic acid; AIBN=2,2′-azobis(2-methylpropionitrile); aq.=aqueous; Boc=butoxycarbonyl; Brettphos=2-(Dicyclohexylphosphino)3,6-dimethoxy-2′,4′,6′-triisopropyl-1,1′-biphenyl; Brettphos Pd G3=[(2-Di-cyclohexylphosphino-3,6-dimethoxy-2′,4′,6′- triisopropyl-1,1′-biphenyl)-2-(2′-amino-1,1′-biphenyl)]palladium(II) methanesulfonate; tert-butylBrettPhos=Di-tert-butyl(2′,4′,6′-triisopropyl-3,6-dimethoxy-[1,1′-biphenyl]-2-yl)phosphine; tert-BuOH=tert-butanol; CDI=1,1′-Carbonyldiimidazole; CPhos=2-Dicyclohexylphosphino-2′,6′-bis(N,N-dimethylamino)biphenyl; Dave-phos-Pd G3=Methanesulfonato 2-dicyclohexylphosphino-2-(N,N-dimethylamino)biphenyl(2′-amino-1,1′-biphenyl-2-yl) palladium(II); DCM=dichloromethane; DEA=Diethylamine; DIAD=Diisopropyl azodicarboxylate; DIPEA=N,N-diisopropylethylamine; Cbz=carboxybenzyl; DMAP=4-(dimethylamino)pyridine; Dess-Martinperiodinane=3-Oxo-115-benzo[d][1,2]iodaoxole-1,1,1(3H)-triyl triacetate; DMF=N,N-dimethylformamide; DMSO=dimethylsulfoxide; EDC=(1-ethyl-3-(3-dimethylaminopropyl)carbodiimide hydrochloride); Ephos=dicyclohexyl(3-isopropoxy-2′,4′,6′-triisopropyl-[1,1′-biphenyl]-2-yl)phosphane; Et₂O=diethyl ether; EtOAc=ethyl acetate; EtOH=ethanol; FSC=flash silica chromatography; h=hour(s); HATU=1-[Bis(dimethylamino)methylene]-1H-1,2,3-triazolo[4,5-b]pyridinium 3-oxid hexafluorophosphate; HOBt=hydroxybenzotriazole; HPLC=high-performance liquid chromatography; IPA=isopropyl alcohol; LHMDS=Lithium bis(trimethylsilyl)amide; MeCN=acetonitrile; MeOH=methanol; mins.=minutes; m/z=mass to charge ratio observed for major mass spectrometry peak(s); MTBE=methyl-tert-butyl-ether; 2-MeTHF=2-Methyltetrahydrofuran; NBS═N bromosuccinimide; NMP=N-methyl-2-pyrrolidone; NMR=nuclear magnetic resonance; [Pd(cinnamyl)Cl]₂=Di-chlorobis[(1,2,3-)-1-phenyl-2-propenyl]dipalladium(II); PdCl₂(dtbpf)=[1,1′-Bis(di-tert-butylphosphino)ferrocene]dichloropalladium(II); Pd₂(dba)₃=tris(dibenzylideneacetone)dipalladium; PdCl₂(PPh₃)₂=Bis(triphenylphosphine)palladium(II) dichloride; Pd-PEPPSI-IHept^{C1}=dichloro[1,3-bis(2,6-di-4-heptylphenyl)imidazol-2-yldiene(3-chloropyridyl)palladium(II); Pd-PEPPSI-IPent=dichloro[1,3-bis(2,6-Di-3-pentylphenyl)imidazol-2-ylidene](3-chloropyridyl)palladium(II), Pd(dppf)₂Cl₂-DCM=1,1′-bis(diphenylphosphino)ferrocene-palladium(II)dichloride dichloromethane complex; Pd(dppf)Cl₂=[1,1′-Bis(diphenylphosphino)ferrocene]dichloropalladium(II); Pd(t-Bu₃P)₂=Bis(tri-tert-butylphosphine)palladium(0); RockPhos=2-di(tert-butyl)phosphino-2′,4′,6′-triisopropyl-3-methoxy-6-methylbiphenyl; RockPhos Pd G3=[(2-di-tert-butylphosphino-3-methoxy-6-methyl-2′,4′,6′-triisopropyl-1,1′-biphenyl)-2-(2-aminobiphenyl)]-palladium(II) methanesulfonate; RT=room temperature (~17-25° C.); RuPhos=2-dicyclohexylphosphino-2′,6′-diisopropoxybiphenyl; RuPhos Pd G3=methanesulfonato(2-dicyclohexylphosphino-2′,6′-di-iso-propoxy-1,1′-biphenyl)(2′-amino-1,1′-biphenyl-2-yl)-palladium(II); TFA=trifluoroacetic acid; THF=tetrahydrofuran; sat.=saturated; SFC=supercritical fluid chromatography; S Phos=2-dicyclohexylphosphino-2,6-di-methyloxy-1,1-biphenyl; Xantphos=4,5-bis(diphenylphosphino)-9,9-dimethylxanthene; XPhos=2-dicyclo-hexylphosphino-2′,4′,6′-triisopropylbiphenyl.

NMR was carried out at 300-500 MHz in deuterated DMSO and at a temperature of 20-30° C. unless otherwise stated. The following standard abbreviations are used for NMR data: s=singlet, d=doublet, m=multiplet, br=broad, dd=doublet of doublets, q=quartet, dt=doublet of triplets, etc.

Preparative reverse phase HPLC (RP HPLC) using decreasingly polar mixture of eluents (e.g. water and MeCN) may typically involve a gradient over 10-20 minutes, at 40-50 mL per minute, from a 95:5 mixture of solvents a 5:95 mixture. The following Column and Eluent conditions are used herein:

Column A: Waters XSelect CSH C18 ODB column, 5 μm silica, 30 mm diameter, 100 mm length

Eluent A: Decreasingly polar mixtures of water (containing 0.1% formic acid) and MeCN

Eluent B: Decreasingly polar mixtures of water (containing 0.1% NH₃) and MeCN

After HPLC (which often involves the presence of formic acid or trifluoroacetic acid in the eluent), the fractions containing desired product were in some cases treated with a suitable base as part of a further work-up step in order to ensure delivery of the title compound as a neutral molecule rather than a salt accordingly where “Basic Work-Up A” is mentioned: fractions containing the desired compound were concentrated to remove MeCN. The resulting, mainly-aqueous fractions were basified with NaHCO₃ solution (e.g. 50 mL) and extracted into DCM (e.g. 3×100 mL). The combined organic solutions were washed with NaCl solution (e.g. 100 mL), dried (e.g. with Na₂SO₄ or MgSO₄) and concentrated to give the title compound.

Concentration/Evaporation: where solutions or mixtures are described as being concentrated or evaporated, this is generally performed on a rotary evaporator under reduced pressure using a warm or hot water bath.

Salts: Where certain compounds were obtained as an acid-addition salt, for example a mono-hydrochloride salt or a bis-hydrochloride salt, the stoichiometry of the salt is assumed, based on the number and nature of the basic groups in the compound, and may not have been determined experimentally e.g. by means of elemental analysis data.

Chemical naming: In general Examples and Intermediate compounds were named using ACD Name, “Structure to Name” part of ChemDraw Ultra (CambridgeSoft) or Biovia Draw 2016.

### Example 1

### Intermediate 1a

### 7-Bromo-4-chloro-1H-indole

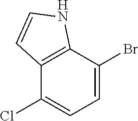 [SMILES: Clc1ccc(Br)c2[nH]ccc12]

1-Bromo-4-chloro-2-nitro-benzene (7×100 g, 422.9 mmol) was added to THF (7×700 mL) followed by dropwise addition of vinylmagnesium bromide (1M, 7×1.69 L) at −60° C. and the mixture was stirred for 1 h. NH₄Cl (aq, 2.0 L) was added to the solution at 0° C. and the product mixtures were combined and extracted into EtOAc (2.0 L). The organic layer was washed with NaCl solution (1.0 L) and the solvent was evaporated to dryness to afford crude product which was purified by column chromatography, elution gradient 10 to 25% Et₂O:EtOAc to give the title compound (260 g, 34%) as a yellow solid. ¹H NMR (CDCl₃) δ 6.64 (1H, s), 6.93 (1H, m), 7.18 (2H, m), 8.35 (1H, s).

### Intermediate 1b

### 7-Bromo-4-chloro-1H-indole-3-carbonitrile

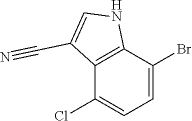 [SMILES: N#Cc1c[nH]c2c(Br)ccc(Cl)c12]

7-Bromo-4-chloro-1H-indole (4×70.0 g, 303.7 mmol) was added to MeCN (4×560 mL), followed by dropwise addition of chlorosulfonyl isocyanate (4×51.6 g, 364.4 mmol, 4×31.6 mL) at 0° C. and stirred for 2 h. DMF (4×266.0 g, 3.64 mol, 4×280.0 mL) was then added dropwise and the solution was continued to stir at 0° C. for 2 h and then to RT for 16 h. Water was added dropwise at 10° C. until a solid formed which was isolated by filtration. The batches of solid were combined and triturated with Et₂O:EtOAc:DCM (4:1:1) to give the title compound (105.0 g, 33.3%) as a white solid. ¹H NMR δ 7.22-7.24 (1H, d, J=8.4 Hz), 7.50-7.52 (1H, d, J=8.4 Hz), 8.46 (1H, s); m/z: ES⁺ [M+H]⁺=255.1.

### Intermediate 1c

### Benzyl 4-(4-bromophenyl)piperidine-1-carboxylate

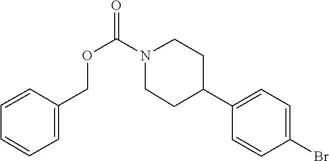 [SMILES: O=C(OCc1ccccc1)N1CCC(c2ccc(Br)cc2)CC1]

Benzyl carbonochloridate (14.27 mL, 96.17 mmol) was added dropwise over 5 mins to a solution of 4-(4-bromophenyl)piperidine (25.0 g, 100.98 mmol) and DIPEA (42.0 mL, 240.43 mmol) in 2-MeTHF (250 mL) at 0° C. The resulting mixture was stirred at RT for 24 h and then quenched with water (250 mL). The layers were separated and the organic layer was washed with water (250 mL), NaCl solution (50 mL), dried using MgSO₄, filtered and evaporated to afford crude product as a colourless oil. The oil was diluted with IPA:water (1:1, 200 mL) and was stirred at RT for 1 h. The resulting solid was collected by filtration, washed with water (2×20 mL) and dried under vacuum at 50° C. for 16 h to give the title compound (32.9 g, 91%) as a white solid. ¹H NMR δ 1.50 (2H, qd), 1.75 (2H, d), 2.72 (1H, m), 2.90 (2H, s), 4.14 (2H, d), 5.10 (2H, s), 7.18-7.26 (2H, m), 7.29-7.44 (5H, m), 7.45-7.53 (2H, m).

### Intermediate 1d

### Benzyl 4-{4-[4-(1,3-dioxolan-2-yl)piperidin-1-yl]phenyl}piperidine-1-carboxylate

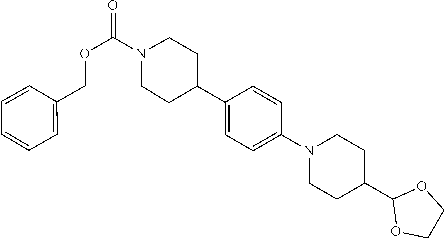 [SMILES: O=C(OCc1ccccc1)N1CCC(c2ccc(N3CCC(C4OCCO4)CC3)cc2)CC1]

To 4-(1,3-dioxolan-2-yl)piperidine (15.02 g, 95.52 mmol), benzyl 4-(4-bromophenyl)piperidine-1-carboxylate (32.5 g, 86.83 mmol) and Cs₂CO₃ (56.6 g, 173.67 mmol) in 1,4-dioxane (325 ml) under nitrogen was added tri-tert-butylphosphonium tetrafluoroborate (2.52 g, 8.68 mmol). The mixture was degassed with nitrogen followed by addition of Pd(t-Bu₃P)₂ (2.21 g, 4.34 mmol). The resulting mixture was stirred at 100° C. for 20 h, cooled to RT and the solid was filtered under vacuum. The solid was washed with 1,4-dioxane (3×65 ml) and the filtrate was evaporated to afford a pale yellow solid. The solid was suspended in cyclopentyl methyl ether:heptane (1:10, 300 ml), stirred at 50° C. for 30 mins and then left to cool to RT for a further 2 h. The solid was filtered to give the title compound (28.9 g, 73.9%) as a cream solid. ¹H NMR δ 1.43 (4H, m), 1.57-1.67 (1H, m), 1.73 (4H, m), 2.54-2.63 (3H, m), 2.89 (2H, s), 3.65 (2H, m), 3.75-3.93 (4H, m), 4.13 (2H, m), 4.61 (1H, d), 5.09 (2H, s), 6.85 (2H, m), 7.06 (2H, m), 7.29-7.44 (5H, m); m/z: ES⁺ [M+H]⁺=451.5.

### Intermediate 1e

### 4-(1,3-Dioxolan-2-yl)-1-[4-(piperidin-4-yl)phenyl]piperidine

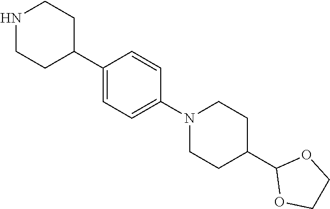 [SMILES: c1cc(N2CCC(C3OCCO3)CC2)ccc1C1CCNCC1]

Benzyl 4-{4-[4-(1,3-dioxolan-2-yl)piperidin-1-yl]phenyl}piperidine-1-carboxylate (762 mg, 1.69 mmol) was dissolved in EtOH (20 mL) and Pd/C (10%, 180 mg, 0.17 mmol) was added under nitrogen. The reaction was stirred under an atmosphere of hydrogen at 4 bar for 16 h. The catalyst was then filtered off through a pad of Celite®, the solvent was removed under reduced pressure and azeotroped with excess MeCN. The product was dried in a vacuum oven for 2 h to give the title compound (449 mg, 84%) as a waxy white solid. ¹H NMR δ 1.33-1.51 (4H, m), 1.54-1.68 (3H, m), 1.72 (2H, d), 2.4-2.46 (1H, m), 2.54-2.64 (4H, m), 3.01 (2H, d), 3.64 (2H, d), 3.74-3.94 (4H, m), 4.61 (1H, d), 6.85 (2H, d), 7.04 (2H, d); m/z: ES⁺ [M+H]⁺=317.7.

### Intermediate 1f

### 4-Chloro-7-(4-{4-[4-(1,3-dioxolan-2-yl)piperidin-1-yl]phenyl}piperidin-1-yl)-1H-indole-3-carbonitrile

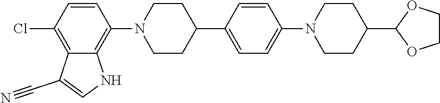 [SMILES: N#Cc1c[nH]c2c(N3CCC(c4ccc(N5CCC(C6OCCO6)CC5)cc4)CC3)ccc(Cl)c12]

[Pd(cinnamyl)Cl]₂ (0.218 g, 0.42 mmol) and CPhos (0.735 g, 1.68 mmol) were added to a degassed mixture of 4-(1,3-dioxolan-2-yl)-1-[4-(4-piperidyl)phenyl]piperidine (11.72 g, 37.03 mmol) and 7-bromo-4-chloro-1H-indole-3-carbonitrile (intermediate 1b) (8.6 g, 33.66 mmol) in 2-MeTHF (86 mL). LHMDS (1M in THF, 118 mL, 117.81 mmol) was added dropwise and the reaction was stirred at 50° C. for 1.5 h. The reaction was left to cool to RT, diluted with water (200 mL) and stirred for 10 mins. A precipitate formed which sat between the two layers, this solid was collected by filtration, was washed with water (25 mL) and vacuum oven dried at 50° C. overnight to give the title compound (13.95 g, 84%) as a cream solid. ¹H NMR δ 1.39 (2H, m), 1.56-1.67 (1H, m), 1.67-1.78 (2H, m), 1.83 (2H, m), 1.88-2.03 (2H, m), 2.58 (3H, td), 2.71-2.82 (2H, m), 3.42 (2H, d), 3.66 (2H, d), 3.72-3.82 (2H, m), 3.82-3.91 (2H, m), 4.60 (1H, d), 6.83 (1H, d), 6.88 (2H, d), 7.04-7.21 (3H, m), 8.28 (1H, s), 12.21 (1H, s); m/z: ES⁺ [M+H]⁺=491.5.

### Intermediate 1g

### 4-Chloro-7-{4-[4-(4-formylpiperidin-1-yl)phenyl]piperidin-1-yl}-1H-indole-3-carbonitrile

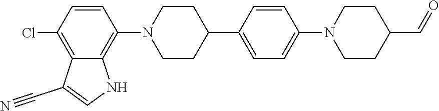 [SMILES: N#Cc1c[nH]c2c(N3CCC(c4ccc(N5CCC(C=O)CC5)cc4)CC3)ccc(Cl)c12]

HCl (2M, 274 mL, 547.83 mmol) was added portion wise to a suspension of 4-chloro-7-(4-{4-[4-(1,3-dioxolan-2-yl)piperidin-1-yl]phenyl}piperidin-1-yl)-1H-indole-3-carbonitrile (13.45 g, 27.39 mmol) in THF (135 mL) at RT. The resulting solution was stirred at 60° C. for 4 h. The resulting solution was pooled in an ice bath and neutralised with NaOH (2M, 180 mL) until ~pH7 is reached. The product was filtered off and the solid was washed with water (20 mL) and dried under vacuum to give the title compound (12.85 g) as a cream solid which was used without further purification. ¹H NMR δ 1.64 (2H, s), 1.83 (2H, d), 1.88-2.06 (4H, m), 2.71-2.92 (4H, m), 3.40 (3H, d), 3.51-3.62 (3H, m), 6.85 (1H, d), 6.99 (2H, s), 7.15 (3H, d), 8.30 (1H, d), 9.63 (1H, s), 12.25 (1H, s); m/z: ES⁺ [M+H]⁺=447.4.

### Intermediate 1h

### 3-(5-Bromo-1-oxo-1,3-dihydro-2H-isoindol-2-yl)piperidine-2,6-dione

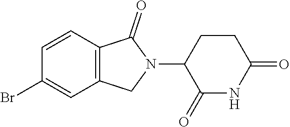 [SMILES: O=C1CCC(N2Cc3cc(Br)ccc3C2=O)C(=O)N1]

DIPEA (25 mL, 143.52 mmol) was added to methyl 4-bromo-2-(bromomethyl)benzoate (14.65 g, 47.57 mmol) and 3-aminopiperidine-2,6-dione hydrochloride (11.74 g, 71.35 mmol) in MeCN (200 mL) under nitrogen. The resulting suspension was stirred at 80° C. for 48 h. The reaction mixture was cooled to RT and filtered. The solid was washed with MeCN (60 mL), MeCN:Et₂O (2:3, 50 mL) and Et₂O (2×50 mL) to give the title compound (13.1 g, 85%) as a dark blue solid; ¹H NMR δ 1.95-2.08 (1H, m), 2.34-2.46 (1H, m), 2.57-2.65 (1H, m), 2.91 (1H, m), 4.35 (1H, d), 4.48 (1H, d), 5.11 (1H, dd), 7.67 (1H, d), 7.72 (1H, dd), 7.83-7.96 (1H, m), 10.98 (1H, s).m/z: ES⁺ [M+H]⁺=323.0.

### Intermediate 1i

### tert-Butyl 4-[2-(2,6-dioxopiperidin-3-yl)-1-oxo-2,3-dihydro-1H-isoindol-5-yl]piperazine-1-carboxylate

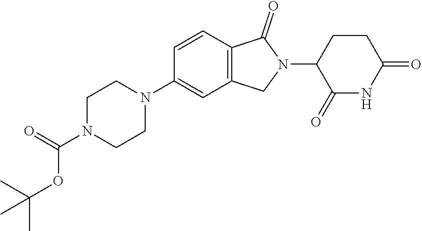 [SMILES: CC(C)(C)OC(=O)N1CCN(c2ccc3c(c2)CN(C2CCC(=O)NC2=O)C3=O)CC1]

Cs₂CO₃ (57.4 g, 176 mmol) and Pd-PEPPSI-IPent (2.33 g, 2.94 mmol) were added in one portion to a degassed solution of tert-butyl piperazine-1-carboxylate (14.22 g, 76.36 mmol) and 3-(5-bromo-1-oxo-1,3-dihydro-2H-isoindol-2-yl)piperidine-2,6-dione (19.0 g, 58.74 mmol) in 1,4-dioxane (590 mL) under nitrogen.

The resulting mixture was stirred at 90° C. for 24 h. The reaction mixture was cooled to RT, diluted with DCM (1L) and washed sequentially with 5% AcOH in water (500 mL) and NaCl solution (500 mL). The organic layer was dried over MgSO₄, filtered and evaporated to afford crude product. The crude product was triturated with EtOAc (250 mL), the solid collected by filtration, washed with Et₂O (100 mL) and dried under vacuum to give the title compound (22.1 g, 88%) as a grey solid; ¹H NMR δ 1.43 (9H, s), 1.96 (1H, d), 2.31-2.41 (1H, m), 2.59 (1H, d), 2.87 (1H, s), 3.29 (4H, d), 3.47 (4H, d), 4.22 (1H, d), 4.34 (1H, d), 5.05 (1H, dd), 7.07 (2H, d), 7.54 (1H, d), 10.92 (1H, s); m/z: ES⁺ [M+H]⁺=429.2.

### Intermediate 1j

### 3-[1-Oxo-5-(piperazin-1-yl)-1,3-dihydro-2H-isoindol-2-yl]piperidine-2,6-dione hydrochloride

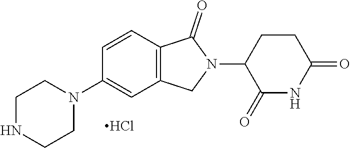 [SMILES: O=C1CCC(N2Cc3cc(N4CCNCC4)ccc3C2=O)C(=O)N1 ; Cl]

A solution of 4M HCl in 1,4-dioxane (8.75 mL, 35.0 mmol) was added to tert-Butyl 4-[2-(2,6-dioxopiperidin-3-yl)-1-oxo-2,3-dihydro-1H-isoindol-5-yl]piperazine-1-carboxylate (1.50 g, 3.50 mmol) in 1,4-dioxane (2 mL) at RT. The reaction was stirred at RT for 1 h. EtOAc (5 mL) was added and the reaction mixture stirred for 10 mins. The resulting precipitate was collected by filtration and the solid washed with EtOAc (2×5 mL) and then dried under vacuum to give the title compound (1.08 g, 85%) as a dark grey solid (HCl salt); ¹H NMR δ 1.97 (1H, dd), 2.36-2.44 (1H, m), 2.60 (1H, d), 2.84-2.99 (1H, m), 3.23 (4H, s), 3.5-3.57 (4H, m), 4.27 (1H, s), 4.34 (1H, s), 5.06 (1H, dd), 7.11-7.18 (2H, m), 7.59 (1H, d), 9.17 (2H, s), 10.93 (1H, s); m/z: ES' [M+H]⁺=329.0.

### Example 1

### 4-Chloro-7-(4-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile

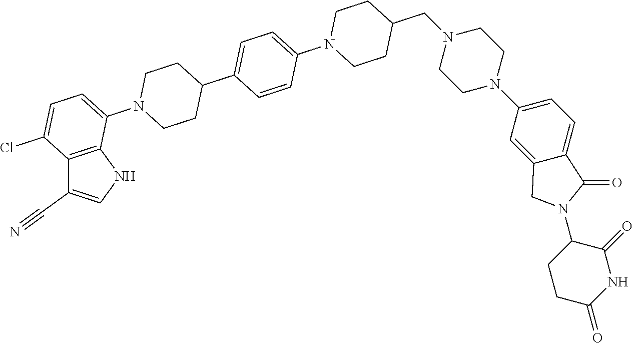 [SMILES: N#Cc1c[nH]c2c(N3CCC(c4ccc(N5CCC(CN6CCN(c7ccc8c(c7)CN(C7CCC(=O)NC7=O)C8=O)CC6)CC5)cc4)CC3)ccc(Cl)c12]

4-Chloro-7-{4-[4-(4-formylpiperidin-1-yl)phenyl]piperidin-1-yl}-1H-indole-3-carbonitrile (intermediate 1g) (9.3 g, 20.81 mmol) and 3-[1-oxo-5-(piperazin-1-yl)-1,3-dihydro-2H-isoindol-2-yl]piperidine-2,6-dione hydrochloride (intermediate 1j) (7.97 g, 21.85 mmol) were stirred in NMP (65.0 mL) at RT followed by DIPEA (3.81 mL, 21.85 mmol). After stirring for 5 h, sodium triacetoxyborohydride (5.29 g, 24.97 mmol) was added in one portion and was stirred at RT 30 mins. The reaction was quenched with water (260 mL), stirred for 30 mins and then filtered under vacuum. The solid was washed with water (10 mL) and was dissolved in IPA:DCM (1:3, 720 mL), was washed with NaHCO₃ solution (180 mL), NaCl solution (180 mL), dried over MgSO₄ and filtered to afford crude product. The crude product was purified by flash silica chromatography, elution gradient 50 to 100% EtOAc in heptane followed by 0-7% MeOH in DCM to afford a cream solid. The solid was suspended in MeCN (65 mL) and stirred at 80° C. for 1 h. The suspension was left to cool to RT for 16 h and was filtered under vacuum to give the title compound (6.06 g, 38.4%) as a white solid. ¹H NMR δ 1.17-1.29 (2H, m), 1.64-1.74 (1H, m), 1.83 (4H, br t), 1.91-2.02 (3H, m), 2.23 (2H, br d), 2.31-2.43 (2H, m), 2.51-2.54 (4H, m), 2.54-2.66 (4H, m), 2.78 (2H, br t), 2.85-2.95 (1H, m), 3.29-3.31 (4H, m), 3.42 (2H, br d), 3.64 (2H, br d), 4.21 (1H, d), 4.33 (1H, d), 5.05 (1H, dd), 6.86 (1H, d), 6.91 (2H, d), 7.05-7.08 (1H, m), 7.07 (1H, s), 7.13 (2H, br d), 7.16 (1H, d), 7.53 (1H, d), 8.32 (1H, s), 10.94 (1H, s), 12.26 (1H, br s); m/z: ES⁺ [M+H]⁺=759.8.

### Example 2

### Intermediate 2a

### 7-Bromo-4-fluoro-1H-indole

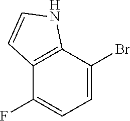 [SMILES: Fc1ccc(Br)c2[nH]ccc12]

1-Bromo-4-fluoro-2-nitro-benzene (6×100.0 g, 454 mmol) was dissolved in THF (6×700.0 mL) at −45° C. followed by dropwise addition of vinylmagnesium bromide (1M, 6×1.82 L) under nitrogen and stirred for 30 mins. NH₄Cl (aq, 2.0 L) was added to the solution and the 6 batches were combined. THF was evaporated and the product was extracted with EtOAc (9.0 L), washed with NaCl solution (1.0 L), dried with Na₂SO₄, filtered and the solvent evaporated. The crude product was purified by column chromatography, elution gradient 0 to 20% Et₂O:EtOAc to give the title compound (148.5 g, 24.3%) as a brown oil. ¹H NMR δ 6.64 (1H, s), 6.78 (1H, m), 7.27 (1H, m), 7.45 (1H, s), 11.65 (1H, s).

### Intermediate 2b

### 7-Bromo-4-fluoro-1H-indole-3-carbonitrile

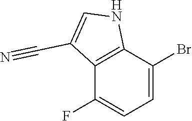 [SMILES: N#Cc1c[nH]c2c(Br)ccc(F)c12]

7-Bromo-4-fluoro-1H-indole (140.0 g, 654 mmol) was added to MeCN (848 mL) followed by chlorosulfonyl isocyanate (111.1 g, 785 mmol, 68.2 mL) dropwise at 0° C. and stirred for 2 h. DMF (574.0 g, 7.85 mol, 604 mL) was added dropwise into the solution at 0° C. and stirred for a further 2 h. Water (1.6 L) was added and the resulting solid was washed with water (100.0 mL) to obtain a yellow solid. The yellow solid was extracted into EtOAc (500.0 mL), dried with Na₂SO₄ and concentrated under reduced pressure. The crude product was triturated with Et₂O:EtOAc (4:1, 800.0 mL) to give the title compound (100.78 g, 62.9%) as a yellow solid. ¹H NMR δ 7.03 (1H, m), 7.50 (1H, m), 8.40 (1H, m), 12.77 (1H, s); m/z: ES⁻ [M−H]⁻=237.0.

### Intermediate 2c

### 7-(4-{4-[4-(1,3-Dioxolan-2-yl)piperidin-1-yl]phenyl}piperidin-1-yl)-4-fluoro-1H-indole-3-carbonitrile

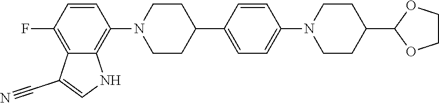 [SMILES: N#Cc1c[nH]c2c(N3CCC(c4ccc(N5CCC(C6OCCO6)CC5)cc4)CC3)ccc(F)c12]

[Pd(cinnamyl)Cl]₂ (0.068 g, 0.13 mmol), CPhos (0.228 g, 0.52 mmol), 4-(1,3-dioxolan-2-yl)-1-[4-(piperidin-4-yl)phenyl]piperidine intermediate 1e (3.64 g, 11.50 mmol) and 7-bromo-4-fluoro-1H-indole-3-carbonitrile intermediate 2b (2.5 g, 10.46 mmol) were degassed in 2-MeTHF (25 mL) for 10 mins. LHMDS (1M in THF, 36.6 mL, 36.60 mmol) was added in one portion and the reaction was stirred at 50° C. for 1.5 h. The reaction mixture was then cooled to RT, diluted with water (65 mL) and stirred for 10 mins. A precipitate formed which sat between the two layers, this was collected by filtration and was washed with water (25 mL) and vacuum oven dried at 50° C. for 16 h to give the title compound (3.41 g, 68.7%) as a cream solid. ¹H NMR δ 1.39 (2H, qd), 1.56-1.67 (1H, m), 1.67-1.77 (2H, m), 1.77-1.86 (2H, m), 1.94 (2H, qd), 2.52-2.64 (3H, m), 2.68-2.8 (2H, m), 3.36 (2H, d), 3.66 (2H, d), 3.72-3.91 (4H, m), 4.60 (1H, d), 6.80 (1H, dd), 6.85-6.94 (3H, m), 7.12 (2H, d), 8.24 (1H, s), 12.19 (1H, s); m/z: ES⁺ [M+H]⁺=475.5.

### Intermediate 2d

### 4-Fluoro-7-{4-[4-(4-formylpiperidin-1-yl)phenyl]piperidin-1-yl}-1H-indole-3-carbonitrile

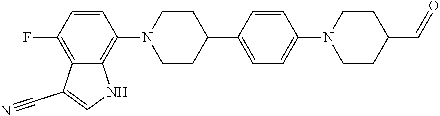 [SMILES: N#Cc1c[nH]c2c(N3CCC(c4ccc(N5CCC(C=O)CC5)cc4)CC3)ccc(F)c12]

To a stirred suspension of 7-(4-{4-[4-(1,3-dioxolan-2-yl)piperidin-1-yl]phenyl}piperidin-1-yl)-4-fluoro-1H-indole-3-carbonitrile (3.41 g, 7.19 mmol) in THF (34.0 mL) at RT was added HCl (2M, 71.9 mL, 143.71 mmol). The mixture was stirred at 60° C. for 2.5 h, cooled in an ice bath and neutralised with NaOH (2M) until pH7 was reached. The mixture was extracted with 2-MeTHF (2×100 mL) then the organic extracts were washed with NaCl solution:water (1:1, 50 mL), dried by passing through a phase separating cartridge, filtered and evaporated to afford the crude product. The crude product was slurried in MeCN (50 mL) at 80° C. for 1 h and then cooled to RT over 16 h. The solid was collected by filtration, washed with excess MeCN and dried under vacuum at 45° C. to give the title compound (2.45 g, 79%) as a cream solid. ¹H NMR δ 1.53-1.66 (2H, m), 1.78-1.88 (2H, m), 1.88-2.03 (4H, m), 2.43-2.48 (1H, m), 2.53-2.63 (1H, m), 2.7-2.85 (4H, m), 3.37 (2H, d), 3.55 (2H, dt), 6.82 (1H, dd), 6.87-6.96 (3H, m), 7.14 (2H, d), 8.26 (1H, d), 9.64 (1H, d), 12.23 (1H, s); m/z: ES⁻ [M−H]⁻=429.3.

### Example 2

### 7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile

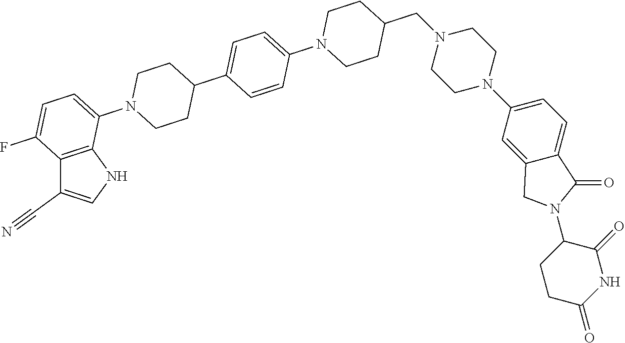 [SMILES: N#Cc1c[nH]c2c(N3CCC(c4ccc(N5CCC(CN6CCN(c7ccc8c(c7)CN(C7CCC(=O)NC7=O)C8=O)CC6)CC5)cc4)CC3)ccc(F)c12]

4-Fluoro-7-{4-[4-(4-formylpiperidin-1-yl)phenyl]piperidin-1-yl}-1H-indole-3-carbonitrile (3.46 g, 8.04 mmol) and 3-[1-oxo-5-(piperazin-1-yl)-1,3-dihydro-2H-isoindol-2-yl]piperidine-2,6-dione HCl intermediate 1j (3.08 g, 8.44 mmol) were suspended in DCM (25.0 mL) and IPA (8.3 mL) at RT. DIPEA (1.47 mL, 8.44 mmol) was added and the suspension was stirred at RT for 2 h. Sodium triacetoxyborohydride (2.04 g, 9.64 mmol) was then added and the suspension was stirred at RT for 5 mins. The reaction mixture was diluted with IPA:DCM (1:3, 135 mL), washed with water (70 mL), NaHCO₃ solution (70 mL), NaCl solution (75 mL) and dried by passing through a phase separating cartridge. The solvent was evaporated to afford the crude product and was purified by flash silica chromatography, elution gradient 50 to 100% EtOAc in heptane followed by 0 to 10% MeOH in DCM. The fractions were evaporated and the product was stirred in MeCN (35 mL) at 80° C. for 1 h and then cooled to RT for 16 h. The solid was collected by filtration, washed with MeCN (10 mL) and dried under vacuum at 45° C. to give the title compound (3.5 g, 58.6%) as a cream solid. ¹H NMR δ 1.16-1.3 (2H, m), 1.63-1.75 (1H, m), 1.76-1.88 (4H, m), 1.9-2.03 (3H, m), 2.23 (2H, d), 2.31-2.42 (1H, m), 2.52 (4H, d), 2.54-2.68 (4H, m), 2.71-2.81 (2H, m), 2.85-2.96 (1H, m), 3.31 (4H, s), 3.37 (2H, d), 3.64 (2H, d), 4.21 (1H, d), 4.33 (1H, d), 5.05 (1H, dd), 6.82 (1H, dd), 6.86-6.97 (3H, m), 7.06 (2H, d), 7.13 (2H, d), 7.52 (1H, d), 8.26 (1H, s), 10.93 (1H, s), 12.23 (1H, s); m/z: ES⁺ [M+H]⁺=743.5.

### Examples 3 & 4

The enantiomers of 7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile (example 2) (30 mg, 0.04 mmol) were separated on a Sepiatec 100 SFC using SFC conditions Regis (R,R) Whelk-01, 21.1×250 mm, 5 micron, 50% MeOH/MeCN 3:7/50% scCO2 at a flow rate of 60 m/min, 40° C. to give, in order of elution, example 3 (isomer 1, 9.0 mg, 30.0%) and example 4 (isomer 2, 7.2 mg, 24.0%) as white solids.

### Example 3

### 7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile [Absolute Stereochemistry not Yet Confirmed]

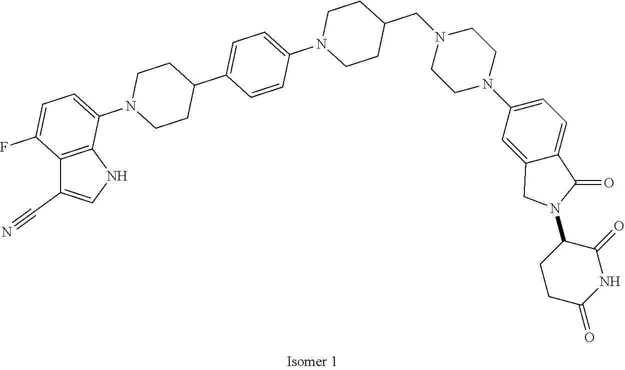

¹H NMR δ 1.17-1.31 (2H, m), 1.64-1.76 (1H, m), 1.78-1.88 (4H, m), 1.89-2.03 (3H, m), 2.23 (2H, d), 2.34-2.4 (1H, m), 2.51-2.53 (4H, m), 2.53-2.55 (1H, m), 2.55-2.59 (1H, m), 2.61 (2H, d), 2.71-2.81 (2H, m), 2.84-2.96 (1H, m), 3.25-3.31 (4H, m), 3.37 (2H, d), 3.64 (2H, d), 4.16-4.37 (2H, m), 5.04 (1H, dd), 6.82 (1H, dd), 6.87-6.96 (3H, m), 7.02-7.09 (2H, m), 7.13 (2H, d), 7.52 (1H, d), 8.26 (1H, d), 10.93 (1H, s), 12.23 (1H, s); m/z: ES⁺ [M+H]⁺=743.5; >99% ee.

### Example 4

### 7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile [Absolute Stereochemistry not Yet Confirmed]

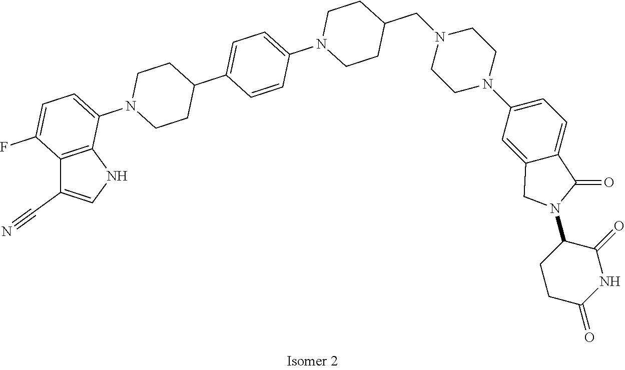

¹H NMR δ 1.24 (3H, s), 1.63-1.76 (1H, m), 1.77-1.88 (4H, m), 1.89-2.03 (3H, m), 2.23 (2H, d), 2.34-2.4 (1H, m), 2.51-2.56 (4H, m), 2.55-2.59 (1H, m), 2.59-2.65 (2H, m), 2.71-2.81 (2H, m), 2.84-2.96 (1H, m), 3.30 (4H, s), 3.34-3.41 (2H, m), 3.6-3.68 (2H, m), 4.16-4.38 (2H, m), 5.04 (1H, dd), 6.82 (1H, dd), 6.88-6.96 (3H, m), 7.03-7.09 (2H, m), 7.13 (2H, d), 7.52 (1H, d), 8.26 (1H, d), 10.93 (1H, s), 12.24 (1H, s); m/z: ES⁺ [M+H]⁺=743.5; >99% cc.

### Example 5

### Intermediate 5a

### tert-Butyl 4-(4-nitro-1H-indol-1-yl)piperidine-1-carboxylate

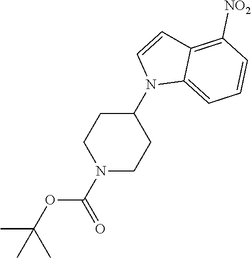 [SMILES: CC(C)(C)OC(=O)N1CCC(n2ccc3c([N+](=O)[O-])cccc32)CC1]

4-Nitro-1H-indole (5×95.0 g, 585 mmol), tert-butyl 4-[(methanesulfonyl)oxy]piperidine-1-carboxylate (5×409.0 g, 1.46 mol) and Cs₂CO₃ (5×573.0 g, 1.76 mol) were added to DMF (1.5 L). The solution was degassed with nitrogen and stirred at 80° C. for 12 h. Water (10.0 L) was added to each mixture and the batches were combined by extracting with EtOAc (6.0 L). The organic layer was washed with NaCl solution (10.0 L×3) and dried with Na₂SO₄. The solvent was evaporated and the crude product was purified by re-crystallisation from MTBE:Et₂O (1:4, 500.0 mL) at RT for 20 mins. The solid was filtered and dried under vacuum to give the title compound (910.0 g) as a brown solid. ¹H NMR δ 1.39 (s, 9H), 1.82-1.93 (m, 4H), 2.85 (s, 2H), 4.08-4.11 (m, 2H), 4.69-4.75 (m, 1H), 7.02 (d, J=3.2 Hz, 1H), 7.32 (t, J=8.4 Hz, 1H), 7.92 (d, J=3.6 Hz, 1H), 8.05 (d, J=7.6 Hz, 1H), 8.13 (d, J=8.4 Hz, 1H).

### Intermediate 5b

### tert-Butyl 4-(4-amino-1H-indol-1-yl)piperidine-1-carboxylate

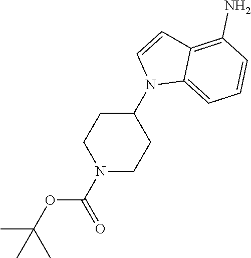 [SMILES: CC(C)(C)OC(=O)N1CCC(n2ccc3c(N)cccc32)CC1]

tert-Butyl 4-(4-nitro-1H-indol-1-yl)piperidine-1-carboxylate (7×140.0 g, 405 mmol) was added to 7 separate solutions of Pd/C (20.0 g, 10% purity) in MeOH:THF (1:1, 1.0 L). The solutions were degassed with nitrogen and stirred for 12 h under an atmosphere of hydrogen (30 PSI). The 7 batches were combined by filtering off Pd/C and evaporating the solvent. The resulting solid was used without further purification to give the title compound (770.0 g, 86%) as a brown solid. ¹H NMR δ 1.43 (s, 9H), 1.86-1.98 (m, 4H), 3.46-3.82 (m, 2H), 4.12 (d, J=10.4 Hz, 2H), 4.59 (t, J=3.6 Hz, 1H), 5.36 (s, 2H), 6.34 (d, J=3.2 Hz, 1H), 6.89 (d, J=7.2 Hz, 1H), 7.16 (t, J=7.6 Hz, 1H), 7.56 (t, J=3.6 Hz, 1H).

### Intermediate 5c

### tert-Butyl 4-{4-[(3-methoxy-3-oxopropyl)amino]-1H-indol-1-yl}piperidine-1-carboxylate

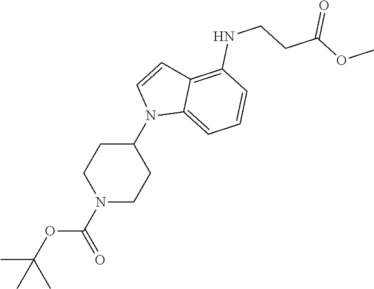 [SMILES: COC(=O)CCNc1cccc2c1ccn2C1CCN(C(=O)OC(C)(C)C)CC1]

tert-Butyl 4-(4-amino-1H-indol-1-yl)piperidine-1-carboxylate (5×175.0 g, 555 mmol) was added to 5 separate portions of MeOH (1.2 L). AcOH (5×499.0 g, 8.32 mol, 476 mL) was added to each mixture followed by methyl acrylate (471.0 g, 5.47 mol, 4923 mL) and was stirred at 80° C. for 18 h. The batches were combined and the solvent was evaporated to afford crude product which was extracted with EtOAc (10.0 L) and washed with NaHCO₃ (15.0 L). The aqueous layer was back extracted with EtOAc (5.0 L×3). The combined organics were washed with NaCl solution and dried with Na₂SO₄. The solvent was evaporated to give the title compound (1.08 kg, crude) as a black oil and was used without further purification. ¹H NMR δ 1.79 (s, 9H), 1.89-1.91 (m, 2H), 1.98 (s, 1H), 2.65 (t, 6.8 Hz, 1H), 2.67 (s, 2H), 3.32-3.42 (m, 1H), 3.51 (s, 1H), 3.61 (s, 2H), 4.02-4.09 (m, 2H), 4.11-4.41 (m, 1H), 6.10 (d, 7.2 Hz, 1H), 6.39-6.54 (m, 1H), 6.78 (d, J=8.4 Hz, 1H), 6.77-6.89 (m, 1H), 7.23 (s, 1H), 11.89 (s, 1H).

### Intermediate 5d

### tert-Butyl 4-{4-[carbamoyl(3-methoxy-3-oxopropyl)amino]-1H-indol-1-yl}piperidine-1-carboxylate

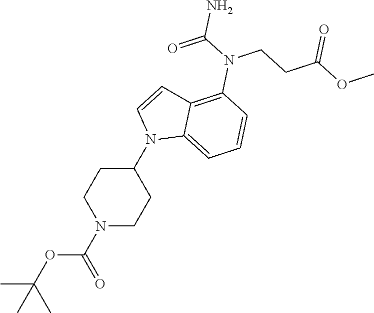 [SMILES: COC(=O)CCN(C(N)=O)c1cccc2c1ccn2C1CCN(C(=O)OC(C)(C)C)CC1]

tert-Butyl 4-{4-[(3-methoxy-3-oxopropyl)amino]-1H-indol-1-yl}piperidine-1-carboxylate (6×200 g, 498 mmol) were added to DCM (6×1.0 L). AcOH (6×2.1 kg, 34.97 mol, 2.0 L) in DCM (6×1.0 L) was added to the mixture followed by potassium cyanate (6×40.4 g, 498 mmol) and stirred at RT for 2 h. The 6 batches were combined and water (5.0 L) was added to the mixture. DCM (8 L×2) was added to the extraction which was washed with NaCl solution. The organic layer was dried with Na₂SO₄ and evaporated. The residue was purified by column chromatography, elution gradient 30:1 to 1:1 Et₂O:EtOAc to give the title compound (260.0 g, 19.6%) as a brown solid. ¹H NMR δ 1.82 (s, 9H), 1.93-1.98 (m, 4H), 2.44-2.50 (m, 2H), 2.98 (s, 2H), 3.46 (s, 3H), 3.84 (s, 2H), 4.66 (d, J=8.4 Hz, 2H), 4.66-4.78 (m, 1H), 5.36 (s, 2H), 6.34 (d, J=3.2 Hz, 1H), 6.90 (d, J=7.6 Hz, 1H), 7.16 (t, J=8.4 Hz, 1H), 7.56 (t, J=4 Hz, 2H).

### Intermediate 5e

### tert-Butyl 4-[4-(2,4-dioxo-1,3-diazinan-1-yl)-1H-indol-1-yl]piperidine-1-carboxylate

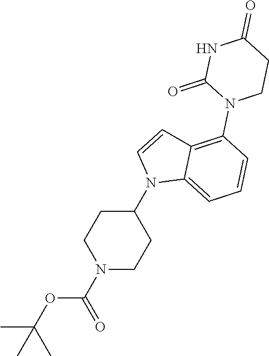 [SMILES: CC(C)(C)OC(=O)N1CCC(n2ccc3c(N4CCC(=O)NC4=O)cccc32)CC1]

tert-Butyl 4-{4-[carbamoyl(3-methoxy-3-oxopropyl)amino]-1H-indol-1-yl}piperidine-1-carboxylate (3×105.0 g, 236 mmol) in MeOH (3×779.0 g, 24.3 mol, 3×984 mL) were added to 3 separate vessels of MeOH (1.0 L) followed by MeONa/MeOH (42.5 g, 236 mmol, 30% purity). The reaction was stirred at RT for 2 h and the combined solids were collected by filtration. The filtrate was evaporated and purified by column chromatography, elution gradient 10:1 DCM:MeOH to give the title compound (180 g, 70.7%) as a white solid. ¹H NMR δ 1.43 (s, 9H), 1.92-1.81 (m, 4H), 2.75 (t, J=6.8 Hz, 2H), 2.7 5 (s, 2H), 3.37-3.77 (m, 2H), 4.12 (d, J=5.6 Hz, 2H), 4.59-4.60 (m, 1H), 6.41 (d, J=3.2 Hz, 1H), 6.96 (d, J=7.6 Hz, 1H), 7.15 (t, J=7.6 Hz, 1H), 7.52-7.54 (m, 2H), 10.3 (s, 1H).

### Intermediate 5f

### 1-[1-(Piperidin-4-yl)-1H-indol-4-yl]-1,3-diazinane-2,4-dione tosylate

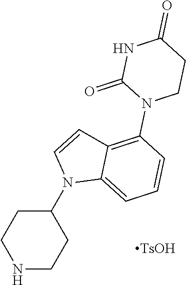

tert-Butyl 4-[4-(2,4-dioxo-1,3-diazinan-1-yl)-1H-indol-1-yl]piperidine-1-carboxylate (140.0 g, 339.41 mmol) in a solution of MeCN (300 mL) was added to p-toluenesulfonic acid hydrate (82.6 g, 434 mmol) in MeCN (560 mL) dropwise at RT. The reaction was stirred at 60° C. for 1 h. p-toluenesulfonic acid hydrate (6.46 g, 33.9 mmol) was added to the mixture and stirred at 60° C. for a further 1 h. The reaction was then filtered and dried under vacuum to give the title compound (120.0 g, 70.8%) as a grey solid. ¹H NMR δ 2.06-2.16 (m, 4H), 2.29 (s, 3H), 2.76 (t, J=6.8 Hz, 2H), 3.19-3.45 (m, 2H), 3.45-3.48 (m, 3H), 3.77 (t, J=6.4 Hz, 2H), 4.42-4.79 (m, 1H), 6.46 (d, J=3.2 Hz, 1H), 6.99 (d, J=7.6 Hz, 1H), 7.10 (d, J=44.0 Hz, 2H), 7.14 (t, J=14.0 Hz, 1H), 7.40 (d, J=3.6 Hz, 1H), 7.52 (d, J=8.0 Hz, 1H), 7.56 (d, J=8.4 Hz, 2H), 8.41 (d, J=10.0 Hz, 1H), 8.68 (d, J=10.0 Hz, 1H), 10.3 (s, 1H); m/z: ES⁺ [M+H]⁺=313.1.

### Example 5

### 4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile

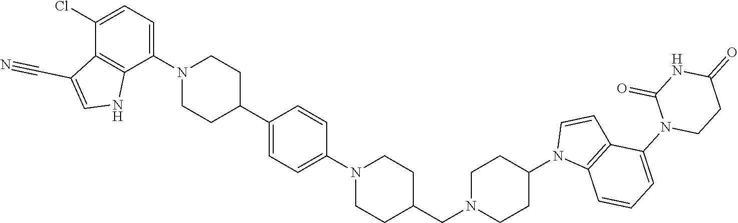 [SMILES: N#Cc1c[nH]c2c(N3CCC(c4ccc(N5CCC(CN6CCC(n7ccc8c(N9CCC(=O)NC9=O)cccc87)CC6)CC5)cc4)CC3)ccc(Cl)c12]

A suspension of intermediate 1g (2.79 g, 6.24 mmol) and 1-[1-(piperidin-4-yl)-1H-indol-4-yl]-1,3-diazinane-2,4-dione TsOH (intermediate 5f) (3.18 g, 6.55 mmol) in NMP (27.0 mL) was stirred at RT for 2.5 h. Sodium triacetoxyborohydride (1.58 g, 7.49 mmol) was added and the solution was continued to stir at RT for 1 h. The reaction mixture was quenched with water (100 mL) and the suspension was stirred at RT for 30 mins. The solid was collected by filtration, washed with water (100 mL) and extracted into DCM:IPA (3:1, 500 mL). The organics were washed with NaHCO₃ solution (150 mL) and NaCl solution (100 mL), dried over a phase separating cartridge and the solvent was evaporated to dryness. The crude product was purified by flash silica chromatography, elution gradient 50 to 100% EtOAc in heptane followed by 100% EtOAc for 15 mins, 0 to 10% MeOH in DCM over 25 mins and 10% MeOH in DCM for 20 mins to afford product as a solid. The product was slurried in MeCN (100 mL) at 80° C. for 1 h and then left to stir to RT for 18 h. The solid was collected by filtration, washing with MeCN and dried under vacuum at 50° C. to give the title compound (2.52 g, 54.3%) as an off white solid. ¹H NMR δ 1.15-1.32 (3H, m), 1.61-1.74 (1H, m), 1.79-1.9 (4H, m), 1.89-2.04 (6H, m), 2.13-2.22 (2H, m), 2.26 (2H, d), 2.51-2.53 (1H, m), 2.53-2.65 (2H, m), 2.73-2.82 (3H, m), 3.01 (2H, d), 3.42 (2H, d), 3.65 (2H, d), 3.78 (2H, t), 4.32-4.43 (1H, m), 6.42 (1H, d), 6.86 (1H, d), 6.88-6.95 (2H, m), 6.96 (1H, d), 7.09-7.2 (4H, m), 7.51 (1H, d), 7.54 (1H, d), 8.31 (1H, s), 10.31 (1H, s), 12.26 (1H, s); m/z: ES⁺ [M+H]⁺=743.4.

### Example 6

### 7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile

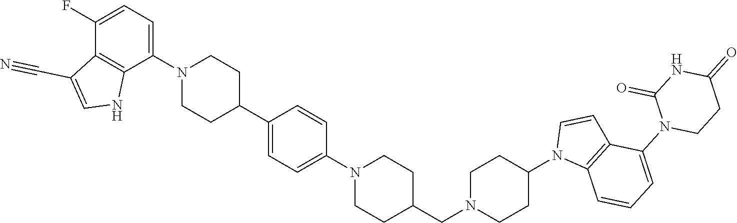 [SMILES: N#Cc1c[nH]c2c(N3CCC(c4ccc(N5CCC(CN6CCC(n7ccc8c(N9CCC(=O)NC9=O)cccc87)CC6)CC5)cc4)CC3)ccc(F)c12]

A suspension of intermediate 2d (2.34 g, 5.44 mmol) and intermediate 5f (2.77 g, 5.71 mmol) in NMP (23.0 mL) was stirred at RT for 2.5 h. Sodium triacetoxyborohydride (1.38 g, 6.52 mmol) was added and the solution was continued to stir at RT for 1 h. The reaction mixture was quenched with water (100 mL) and the suspension was stirred at RT for 30 mins. The solid was collected by filtration and was washed with water. The solid was slurried in MeCN (50 mL) at 80° C. for 1 h and then left to cool to RT for 18 h. The suspension was filtered under vacuum and washed with MeCN to afford crude product. The crude product was purified by flash silica chromatography, elution gradient 50 to 100% EtOAc in heptane over 15 mins followed by 100% EtOAc for 15 mins, 0 to 10% MeOH in DCM over 25 mins and 10% MeOH in DCM for 20 mins to afford product as a solid. The product was slurried in MeCN (100 mL) at 80° C. for 1 h and then left to stir to RT for 18 h. The solid was collected by filtration, washing with MeCN and dried under vacuum at 50° C. to give the title compound (2.22 g, 56.2%) as an off white solid. ¹H NMR δ 1.18-1.3 (2H, m), 1.6-1.73 (1H, m), 1.84 (4H, d), 1.9-2.06 (6H, m), 2.13-2.23 (2H, m), 2.26 (2H, d), 2.55-2.58 (1H, m), 2.58-2.68 (2H, m), 2.71-2.82 (4H, m), 3.01 (2H, d), 3.38 (2H, d), 3.65 (2H, d), 3.78 (2H, t), 4.3-4.46 (1H, m), 6.42 (1H, d), 6.82 (1H, dd), 6.86-7.04 (4H, m), 7.14 (3H, t), 7.44-7.6 (2H, m), 8.26 (1H, s), 10.31 (1H, s), 12.24 (1H, s); m/z: ES⁺ [M+H]⁺=727.5.

### Example 7

### Intermediate 7a

### Methyl 4-bromo-2-methoxy-6-methylbenzoate

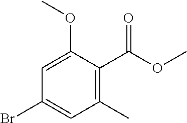

A 25% methanolic solution of sodium methanolate (512 μL, 2.24 mmol) was added dropwise to a stirred solution of methyl 4-bromo-2-fluoro-6-methylbenzoate (527 mg, 2.13 mmol) in DMF (10 mL) at RT under nitrogen. The resulting mixture was stirred at RT for 18 h. The reaction mixture was cooled to 0° C. and quenched with EtOAc (20 mL) and HCl (1M, 10 mL). The phases were separated and the aqueous phase was extracted with EtOAc (2×30 mL). The organic phases were combined, dried over MgSO₄, filtered and evaporated to afford crude product. The crude product was purified by flash silica chromatography, elution gradient 0 to 25% EtOAc in heptane to give the title compound (0.410 g, 74%) as a colourless oil which solidified on standing; ¹H NMR (CDCl₃) δ 2.25 (3H, s), 3.81 (3H, s), 3.90 (3H, s), 6.91 (1H, d), 6.98 (1H, dd); m/z: ES⁺ [M+H]⁺=227.3.

### Intermediate 7b

### 3-(5-Bromo-7-methoxy-1-oxo-1,3-dihydro-2H-isoindol-2-yl)piperidine-2,6-dione

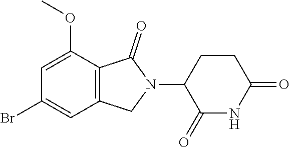 [SMILES: COc1cc(Br)cc2c1C(=O)N(C1CCC(=O)NC1=O)C2]

NBS (1.57 g, 8.84 mmol) was added to a stirred solution of methyl 4-bromo-2-methoxy-6-methylbenzoate (1.43 g, 5.53 mmol) and AIBN (0.182 g, 1.11 mmol) in t-butyl acetate (20 mL). The reaction was stirred at 100° C. for 3 h. The reaction was cooled to RT, diluted with EtOAc (50 mL) and washed with water (50 mL). The organic layer was passed through a phase sep cartridge and concentrated. The crude product was purified by flash silica chromatography, elution gradient 0 to 15% EtOAc in heptane to afford 4-bromo-2-(bromomethyl)-6-methoxybenzoate (1.49 g, 80%) as a yellow gum, that was 70% pure. The solid was added to MeCN (20 mL) followed by DIPEA (1.65 ml, 9.29 mmol) and 3-aminopiperidine-2,6-dione hydrochloride (0.510 g, 3.10 mmol) at RT. The resulting solution was stirred at 80° C. for 16 h. The reaction mixture was cooled to 0° C. and the solid was collected by filtration. The solid was washed with MeCN (50 mL) and Et₂O (50 mL) and dried under vacuum to give the title compound (0.761 g, 39% over 2 steps) as a mauve solid. ¹H NMR δ 1.97 (1H, dtd), 2.34 (1H, qd), 2.54-2.63 (1H, m), 2.90 (1H, m), 3.90 (3H, s), 4.25 (1H, d), 4.38 (1H, d), 5.02 (1H, dd), 7.26 (1H, d), 7.39 (1H, d), 10.94 (1H, s); m/z: ES⁺ [M+H]⁺=353.0.

### Intermediate 7c

### tert-Butyl 4-[2-(2,6-dioxopiperidin-3-yl)-7-methoxy-1-oxo-2,3-dihydro-1H-isoindol-5-yl]piperazine-1-carboxylate

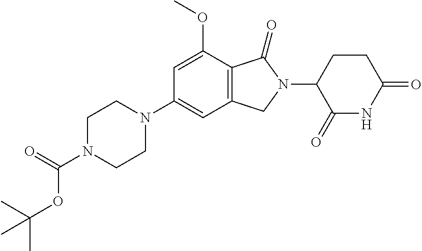 [SMILES: COc1cc(N2CCN(C(=O)OC(C)(C)C)CC2)cc2c1C(=O)N(C1CCC(=O)NC1=O)C2]

Pd-PEPPSI-IHept^{C1} (0.380 g, 0.39 mmol) was added to tert-butyl piperazine-1-carboxylate (2.18 g, 11.72 mmol), Cs₂CO₃ (3.82 g, 11.72 mmol) and 3-(5-bromo-7-methoxy-1-oxo-1,3-dihydro-2H-isoindol-2-yl)piperidine-2,6-dione (1.38 g, 3.91 mmol) in degassed 1,4-dioxane (39 mL) at RT under nitrogen. The resulting suspension was stirred at 100° C. for 6 h. The reaction mixture was diluted with DCM (100 mL) and washed sequentially with 5% AcOH in water (100 mL), water (100 mL), NaHCO₃ solution (100 mL) and NaCl solution (100 mL). The organic layer was dried with MgSO₄, filtered and evaporated to afford crude product. The crude product was triturated with EtOAc (40 mL) and washed with Et₂O (50 mL) to give a solid which was collected by filtration and dried under vacuum to give the title compound (1.15 g, 64%) as a dark grey solid. ¹H NMR δ 1.43 (9H, s), 1.79-1.97 (1H, m), 2.28 (1H, dd), 2.53-2.62 (1H, m), 2.76-2.94 (1H, m), 3.32 (4H, s), 3.41-3.54 (4H, m), 3.85 (3H, s), 4.12 (1H, d), 4.24 (1H, d), 4.95 (1H, dd), 6.51 (1H, d), 6.62 (1H, s), 10.87 (1H, s); m/z: ES⁺ [M+H]⁺=459.2.

### Example 7

### 4-Chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1H-indole-3-carbonitrile

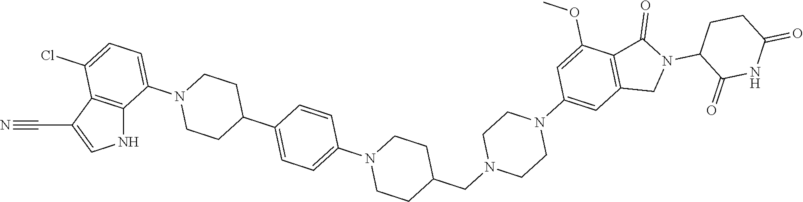 [SMILES: COc1cc(N2CCN(CC3CCN(c4ccc(C5CCN(c6ccc(Cl)c7c(C#N)c[nH]c67)CC5)cc4)CC3)CC2)cc2c1C(=O)N(C1CCC(=O)NC1=O)C2]

Intermediate 1f (780 mg, 1.59 mmol) and tert-butyl 4-[2-(2,6-dioxopiperidin-3-yl)-7-methoxy-1-oxo-2,3-dihydro-1H-isoindol-5-yl]piperazine-1-carboxylate (874 mg, 1.91 mmol) were heated in formic acid (5 mL, 132.54 mmol) at 60° C. for 2 h. The reaction mixture was concentrated and the crude residue was suspended in NMP (5 mL) at RT, stirred for 5 minutes and sodium triacetoxyborohydride (673 mg, 3.18 mmol) was added. The resulting suspension was stirred at RT for 10 mins. The reaction mixture was poured into NaHCO₃ solution (20 mL) and the resulting solid was collected by filtration, washed with MeCN (20 mL) and EtOAc (20 mL). The solid was purified by preparative HPLC (Column A, Eluent A). The fractions containing product were evaporated and dissolved in DCM (250 mL), washed with NaHCO₃ solution (100 mL) and NaCl solution (100 mL). The organic layer was dried over MgSO₄ and evaporated to dryness to give the title compound (350 mg, 27.9%) as a white solid. ¹H NMR δ 1.19-1.3 (2H, m), 1.70 (1H, s), 1.79-1.88 (4H, m), 1.9-2.03 (3H, m), 2.24 (2H, d), 2.31 (1H, d), 2.53 (4H, d), 2.60 (4H, dd), 2.79 (2H, t), 2.85-2.97 (1H, m), 3.43 (2H, d), 3.65 (2H, d), 3.84 (3H, s), 4.11 (1H, d), 4.24 (1H, d), 4.97 (1H, dd), 6.50 (1H, s), 6.62 (1H, s), 6.89 (3H, dd), 7.16 (3H, dd), 8.32 (1H, s), 10.90 (1H, s), 12.26 (1H, s); m/z: ES⁺ [M+H]⁺=790.0.

### Example 8

### Intermediate 8a

### 7-Bromo-4-methyl-1H-indole-3-carbonitrile

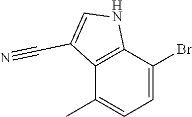

Chlorosulfonyl isocyanate (1.9 mL, 22.61 mmol) was added dropwise to a cooled solution of 7-bromo-4-methyl-1H-indole (5.0 g, 23.8 mmol) in MeCN (94 mL) and DMF (23 mL) at 0° C. The reaction was stirred to RT for 1.5 h. The reaction was then quenched with NaHCO₃ solution (50 mL) and diluted with DCM (100 mL). The organic layer was dried over a phase separating cartridge and the solvent evaporated to afford crude product which was purified by column chromatography, elution gradient 0 to 30% EtOAc in heptane to give the title compound (3.93 g, 70.2%) as a cream solid. ¹H NMR (CDCl₃) δ 2.75 (3H, d), 6.92 (1H, m), 7.35 (1H, m), 7.78 (1H, m), 8.73 (1H, s); m/z: ES⁺ [M+H]⁺=235.0.

### Intermediate 8b

### 7-(4-{4-[4-(1,3-Dioxolan-2-yl)piperidin-1-yl]phenyl}piperidin-1-yl)-4-methyl-1H-indole-3-carbonitrile

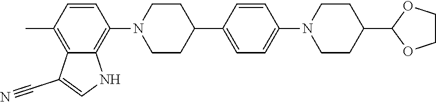 [SMILES: Cc1ccc(N2CCC(c3ccc(N4CCC(C5OCCO5)CC4)cc3)CC2)c2[nH]cc(C#N)c12]

To a mixture of 7-bromo-4-methyl-1H-indole-3-carbonitrile (459 mg, 1.95 mmol), intermediate 1e (1.23 g, 3.90 mmol), RuPhos (27.3 mg, 0.06 mmol) and RuPhos Pd G3 (49.0 mg, 0.06 mmol) in 1,4-dioxane (3 mL) under nitrogen was added LHMDS (1M in THF, 10.63 mL, 10.63 mmol) and the reaction was stirred at 90° C. for 1 h. The reaction mixture was cooled to RT, diluted with EtOAc (50 mL), washed with water (2×50 mL) and NaCl solution (25 mL). The organic layer was dried with a phase separating cartridge and evaporated to afford crude product. The crude product was purified by flash silica chromatography, elution gradient 0 to 100% EtOAc in heptane give the title compound (0.388 g, 42.2%) as a yellow solid. ¹H NMR δ 1.37-1.48 (3H, m), 1.57-1.7 (3H, m), 1.7-1.79 (3H, m), 2.57-2.63 (4H, m), 2.69-2.79 (2H, m), 3.38 (2H, d), 3.68 (2H, d), 3.74-3.95 (6H, m), 4.58-4.66 (1H, m), 6.78 (1H, d), 6.91 (3H, d), 7.14 (2H, d), 8.12-8.21 (1H, m), 11.90 (1H, s); m/z: ES⁺ [M+H]⁺=471.4.

### Example 8

### 7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-methyl-1H-indole-3-carbonitrile

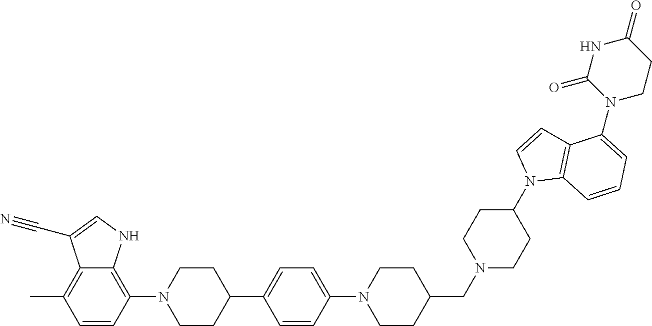 [SMILES: Cc1ccc(N2CCC(c3ccc(N4CCC(CN5CCC(n6ccc7c(N8CCC(=O)NC8=O)cccc76)CC5)CC4)cc3)CC2)c2[nH]cc(C#N)c12]

7-(4-{4-[4-(1,3-Dioxolan-2-yl)piperidin-1-yl]phenyl}piperidin-1-yl)-4-methyl-1H-indole-3-carbonitrile (424 mg, 0.76 mmol) was heated in formic acid (5 mL, 129.46 mmol) at 40° C. for 2.5 h. The reaction mixture was concentrated and the crude residue was suspended in NMP (2 mL) at RT. Intermediate 5f (528 mg, 1.14 mmol) was added and the reaction mixture was stirred at RT for 5 mins. Sodium triacetoxyborohydride (565 mg, 2.67 mmol) was added and the resulting suspension was continued to stir at RT for 10 mins. The reaction mixture was quenched with water (10 mL) and the product was extracted with DCM:IPA (9:1, 25 mL). The organic layer was washed with NaHCO₃ solution, NaCl solution (10 mL) and dried over a phase separating cartridge. The solvent was evaporated and the crude product was purified by flash silica chromatography, elution gradient 0 to 25% IPA in DCM to give the title compound (291 mg, 0.403 mmol, 52.9%) as a white solid. ¹H NMR δ 1.16-1.31 (3H, m), 1.61-1.75 (1H, m), 1.84 (4H, d), 1.89-2.09 (6H, m), 2.13-2.31 (4H, m), 2.55-2.61 (4H, m), 2.61-2.7 (1H, m), 2.7-2.82 (4H, m), 3.02 (2H, d), 3.38 (2H, d), 3.65 (2H, d), 3.72-3.83 (2H, m), 4.39 (1H, s), 6.42 (1H, d), 6.77 (1H, d), 6.84-6.94 (3H, m), 6.96 (1H, dd), 7.11-7.18 (3H, m), 7.51 (1H, d), 7.54 (1H, d), 8.16 (1H, d), 10.31 (1H, s), 11.89 (1H, s); m/z: ES⁺ [M+H]⁺=723.5.

### Example 9

### Intermediate 9a

### 7-Bromo-4-chloro-1H-indazole

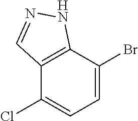 [SMILES: Clc1ccc(Br)c2[nH]ncc12]

3-Bromo-6-chloro-2-fluorobenzaldehyde (23.0 g, 96.86 mmol) was added to 1,2-dimethoxyethane (230 mL) and hydrazine hydrate (14.55 g, 290.58 mmol) was added. The mixture was stirred at reflux for 36 h, cooled to RT and then poured into rapidly stirred water (460 mL) for 30 mins. The resulting solid was filtered and washed with water (2×200 mL) and dried in a vacuum oven at 50° C. for 24 h to give the title compound (21.80 g, 97%) as a white solid. ¹H NMR δ 7.16 (1H, d), 7.60 (1H, d), 8.30 (1H, s), 13.02 (1H, s); m/z: ES⁺ [M+H]⁺=231.1.

### Intermediate 9b

### 7-Bromo-4-chloro-3-iodo-1H-indazole

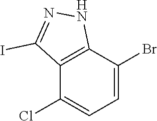 [SMILES: Clc1ccc(Br)c2[nH]nc(I)c12]

7-Bromo-4-chloro-1H-indazole (21.0 g, 90.72 mmol) was added to DMF (100 mL) and potassium hydroxide (20.36 g, 362.89 mmol) and cooled to 0° C. Iodine (29.9 g, 117.94 mmol) was added and after 5 mins the reaction was stirred to RT for 30 mins. The reaction was quenched with sodium thiosulfate (20% aq, 250 mL) and extracted with EtOAc (250 mL). The organic layer was washed with lithium chloride (10% aq, 250 mL), water (2×250 mL), NaCl solution (100 mL), dried over a phase separating cartridge and the solvent evaporated to give the title compound (31.2 g, 96%) as a cream solid. ¹H NMR δ 7.15 (1H, d), 7.63 (1H, d), 14.23 (1H, s); m/z: ES⁻ [M−H]⁻=355.0.

### Intermediate 9c

### 7-Bromo-4-chloro-1H-indazole-3-carbonitrile

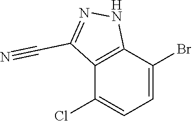 [SMILES: N#Cc1n[nH]c2c(Br)ccc(Cl)c12]

7-Bromo-4-chloro-3-iodo-1H-indazole (10.0 g, 27.98 mmol) and potassium hexacyanoferrate(II) trihydrate (4.73 g, 11.19 mmol) were added to dimethylacetamide (80 mL) under nitrogen and stirred at 60° C. for 10 mins. Water (60 mL) was added followed by Xantphos (0.810 g, 1.40 mmol) and allylpalladium(II) chloride dimer (0.256 g, 0.70 mmol) and the mixture was stirred at 95° C. for 4.5 h. The reaction was cooled to RT, filtered through Celite® and washed with 2-MeTHF (200 mL). The mother liquor was diluted with water (200 mL) and washed with water (2×200 mL), NaCl solution (100 mL), dried over a phase separating cartridge and the solvent was evaporated. The crude product was suspended in MeCN (50 mL) and stirred at reflux. Water (50 mL) was added followed by MeCN (20 mL). The solution was decanted hot through cotton wool and left to cool to RT. The resulting solid was filtered, dried under vacuum and slurried in DCM (20 mL) for 5 mins. The resulting solid was dried under vacuum to give the title compound (4.55 g, 63.4%) as an off-white solid. ¹H NMR δ 7.40 (1H, d), 7.79 (1H, d), 15.17 (1H, s); m/z: ES⁻ [M−H]⁻=254.0.

### Intermediate 9d

### Benzyl (3S)-3-(4-bromophenyl)piperidine-1-carboxylate

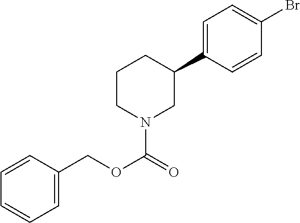 [SMILES: O=C(OCc1ccccc1)N1CCC[C@@H](c2ccc(Br)cc2)C1]

K₂CO₃ (2M, 159.0 mL, 318.01 mmol) was added portion wise to a solution of (S)-3-(4-bromophenyl)piperidine oxalate (35.0 g, 106.00 mmol) in THF (250 mL) and water (100 mL) at RT. 1-{[(Benzyloxy)carbonyl]oxy}pyrrolidine-2,5-dione (26.4 g, 106.00 mmol) was then added portion wise and was stirred at RT for 18 h. The mixture was diluted with EtOAc (500 mL) and water (250 mL), stirred for 10 mins and the layers separated. The organic layer was washed with citric acid (1M, 250 mL), NaCl solution (200 mL), dried over a phase separating cartridge and evaporated to dryness to afford crude product. The crude product was dissolved in EtOH (100 mL) and water (300 mL), stirred at 60° C. and then cooled to RT. The resulting solid was filtered under vacuum and dried in a vacuum oven at 45° C. to give the title compound (37.6 g, 94%) as a cream solid. ¹H NMR δ 1.48 (1H, m), 1.63 (1H, m), 1.72 (1H, m), 1.83-1.93 (1H, m), 2.65 (1H, tt), 2.87 (2H, s), 3.96-4.08 (2H, m), 5.10 (2H, s), 7.24 (2H, d), 7.28-7.42 (5H, m), 7.50 (2H, d); m/z: ES⁺ [M+H]⁺=374.1.

### Intermediate 9e

### Benzyl (3S)-3-{4-[4-(1,3-dioxolan-2-yl)piperidin-1-yl]phenyl}piperidine-1-carboxylate

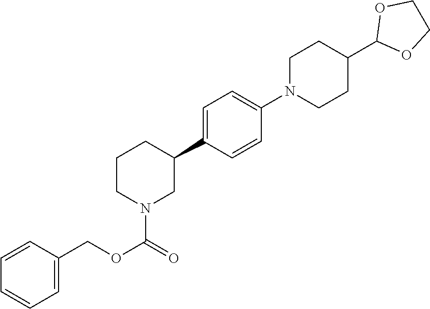 [SMILES: O=C(OCc1ccccc1)N1CCC[C@@H](c2ccc(N3CCC(C4OCCO4)CC3)cc2)C1]

4-(1,3-Dioxolan-2-yl)piperidine (25.9 g, 164.74 mmol), benzyl (3S)-3-(4-bromophenyl)piperidine-1-carboxylate (56.1 g, 149.77 mmol) and Cs₂CO₃ (98.0 g, 299.54 mmol) were degassed with nitrogen in 1,4-dioxane (500 mL) for 10 mins. Pd(t-Bu₃P)₂ (3.83 g, 7.49 mmol) and tri-tert-butylphosphonium tetrafluoroborate (4.35 g, 14.98 mmol) were then added and the mixture stirred at 100° C. for 20 h. The reaction mixture was cooled to RT and the solid was filtered through Celite®. The solid was washed with 1,4-dioxane (200 mL) and the filtrate was evaporated to afford crude product. The crude product was purified by flash silica chromatography, elution gradient 10 to 50% EtOAc in heptane to give the title compound (28.5 g, 42.2%) as a cream solid. ¹H NMR δ 1.33-1.52 (3H, m), 1.55-1.68 (2H, m), 1.68-1.78 (3H, m), 1.85 (1H, d), 2.54 (1H, s), 2.59 (2H, td), 2.84 (2H, s), 3.67 (2H, d), 3.74-3.83 (2H, m), 3.83-3.92 (2H, m), 3.94-4.09 (2H, m), 4.61 (1H, d), 5.09 (2H, s), 6.87 (2H, d), 7.08 (2H, d), 7.27-7.45 (5H, m); m/z: ES⁺ [M+H]⁺=451.3.

### Intermediate 9f

### 4-(1,3-Dioxolan-2-yl)-1-{4-[(3S)-piperidin-3-yl]phenyl}piperidine

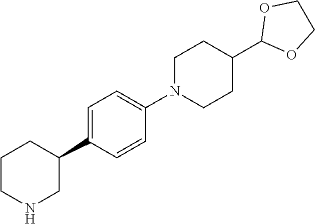 [SMILES: c1cc(N2CCC(C3OCCO3)CC2)ccc1[C@@H]1CCCNC1]

Benzyl (3S)-3-{4-[4-(1,3-dioxolan-2-yl)piperidin-1-yl]phenyl}piperidine-1-carboxylate (27.5 g, 61.03 mmol) was suspended in EtOH (100 mL) and DCM (20 mL). Pd/C (10%, 2.6 g, 2.44 mmol) was added and the reaction was hydrogenated at RT with 2 bar pressure for 16 h. DCM (50 mL) was added to the reaction mixture and was filtered through Celite®. The filtrate was evaporated to dryness and stirred in MTBE (50 mL) for 16 h, filtered and dried under vacuum to give the title compound (14.95 g, 77%) as a cream solid. ¹H NMR δ 1.33-1.44 (2H, m), 1.48 (2H, d), 1.62 (2H, m), 1.72 (2H, d), 1.82 (1H, d), 2.47 (4H, d), 2.58 (2H, m), 2.94 (2H, d), 3.65 (2H, d), 3.75-3.83 (2H, m), 3.83-3.9 (2H, m), 4.61 (1H, d), 6.84 (2H, d), 7.04 (2H, d); m/z: ES⁺ [M+H]⁺=317.2.

### Intermediate 9g

### 4-Chloro-7-[(3S)-3-{4-[4-(1,3-dioxolan-2-yl)piperidin-1-yl]phenyl}piperidin-1-yl]-1H-indazole-3-carbonitrile

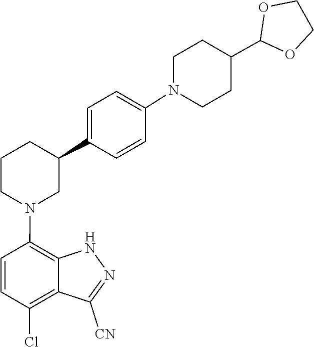 [SMILES: N#Cc1n[nH]c2c(N3CCC[C@@H](c4ccc(N5CCC(C6OCCO6)CC5)cc4)C3)ccc(Cl)c12]

Intermediate 9c (2.3 g, 8.98 mmol) and 4-(1,3-dioxolan-2-yl)-1-{4-[(3S)-piperidin-3-yl]phenyl}piperidine (2.90 g, 9.16 mmol) were suspended in 2-MeTHF (25 mL). The mixture was degassed with nitrogen for 10 mins followed by addition of CPhos (0.39 g, 0.90 mmol) and [Pd(cinnamyl)Cl]₂ (0.23 g, 0.45 mmol). LHMDS (1M in THF, 31.4 mL, 31.45 mmol) was added and the reaction was stirred at 50° C. for 1 h. The reaction was then cooled to RT and partitioned between water (25 mL) and 2-MeTHF (25 mL). The organic layer was washed with NaCl solution (25.0 mL), dried through a phase separating cartridge and the solvent was evaporated to afford crude product. The crude product was purified by flash silica chromatography, elution gradient 0 to 100% EtOAc in heptane to give the title compound (2.46 g, 55.6%) as a pale orange solid. ¹H NMR δ 1.39 (2H, m), 1.62 (2H, m), 1.72 (2H, d), 1.84-1.98 (3H, m), 2.59 (2H, m), 2.66-2.8 (2H, m), 3.00 (1H, t), 3.47 (2H, t), 3.67 (2H, d), 3.75-3.83 (2H, m), 3.83-3.9 (2H, m), 4.61 (1H, d), 6.87 (2H, d), 6.94 (1H, d), 7.18 (2H, d), 7.28 (1H, d), 14.69 (1H, s); m/z: ES⁺ [M+H]⁺=492.3.

### Intermediate 9h

### 4-Chloro-7-{(3S)-3-[4-(4-formylpiperidin-1-yl)phenyl]piperidin-1-yl}-1H-indazole-3-carbonitrile

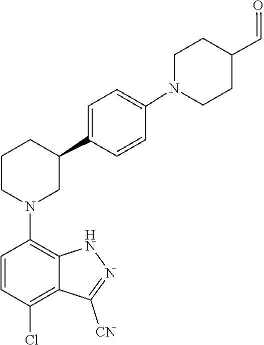 [SMILES: N#Cc1n[nH]c2c(N3CCC[C@@H](c4ccc(N5CCC(C=O)CC5)cc4)C3)ccc(Cl)c12]

To a stirred solution of 4-chloro-7-[(3S)-3-{4-[4-(1,3-dioxolan-2-yl)piperidin-1-yl]phenyl}piperidin-1-yl]-1H-indazole-3-carbonitrile (3.15 g, 6.40 mmol) in THF (32.0 mL) was added HCl (2M, 64.0 mL, 128.04 mmol). The reaction mixture was stirred at 60° C. for 4 h and then cooled to 0° C. in an ice bath. The mixture was neutralised to pH7 with NaOH (2M), extracted with 2-MeTHF (100 mL), washed with water (100 mL), NaCl solution (50 mL), dried over a phase separating cartridge and the solvent was evaporated to afford crude product. The crude product was purified by flash silica chromatography, elution gradient 0 to 100% EtOAc in heptane to give the title compound (2.5 g, 87%) as a yellow solid. ¹H NMR δ 1.58 (3H, m), 1.83-1.98 (5H, m), 2.46 (1H, m), 2.66-2.82 (4H, m), 2.94-3.06 (1H, m), 3.4-3.6 (4H, m), 6.89 (2H, d), 6.93 (1H, d), 7.18 (2H, d), 7.27 (1H, d), 9.63 (1H, d), 14.69 (1H, s); m/z: ES⁺ [M+H]⁺=448.3.

### Example 9

### 4-Chloro-7-[(3S)-3-{4-[4-({4-[4-(2,4-dioxo-1,3-diazinan-1-yl)-1H-indol-1-yl]piperidin-1-yl}methyl)piperidin-1-ylphenyl}piperidin-1-yl]-1H-indazole-3-carbonitrile

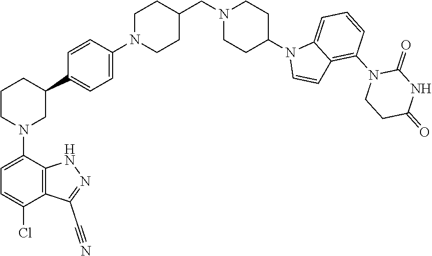 [SMILES: N#Cc1n[nH]c2c(N3CCC[C@@H](c4ccc(N5CCC(CN6CCC(n7ccc8c(N9CCC(=O)NC9=O)cccc87)CC6)CC5)cc4)C3)ccc(Cl)c12]

A suspension of 4-chloro-7-{(3S)-3-[4-(4-formylpiperidin-1-yl)phenyl]piperidin-1-yl}-1H-indazole-3-carbonitrile (9.22 g, 20.58 mmol) and intermediate 5f (10.47 g, 21.61 mmol) in NMP (92 mL) was stirred at RT for 18 h. Sodium triacetoxyborohydride (5.23 g, 24.70 mmol) was added and the solution was continued to stir at RT for 2 h. The reaction mixture was quenched with water (250 mL) and the suspension was stirred at RT for 30 mins. The solid was collected by filtration, was washed with excess water and extracted with DCM:IPA (3:1, 250 mL). The organic layer was washed with NaHCO₃ solution (100 mL), NaCl solution (100 mL) and dried over a phase separating cartridge. The solvent was evaporated and the crude product was purified by flash silica chromatography, elution gradient 0 to 100% EtOAc in heptane followed by 0 to 10% MeOH in EtOAc. The product was then slurried in MeCN (225 mL) at 80° C. for 3 h and then left to cool to RT for 16 h. The solid was collected by filtration, washing with excess MeCN and dried under vacuum at 45° C. to give the title compound (11.57 g, 76%) as a pale orange solid. ¹H NMR δ 1.17-1.29 (2H, m), 1.53-1.72 (2H, m), 1.79-1.85 (2H, m), 1.85-1.99 (5H, m), 1.98-2.06 (2H, m), 2.15-2.24 (2H, m), 2.27 (2H, d), 2.57-2.65 (2H, m), 2.66-2.79 (4H, m), 2.95-3.05 (3H, m), 3.43-3.55 (2H, m), 3.63 (2H, d), 3.78 (2H, t), 4.33-4.43 (1H, m), 6.41 (1H, d), 6.88 (2H, d), 6.92 (1H, d), 6.96 (1H, d), 7.12-7.2 (3H, m), 7.26 (1H, d), 7.51 (1H, d), 7.53 (1H, d), 10.31 (1H, s), 14.55 (1H, s); m/z: ES⁺ [M+H]⁺=744.4.

### Example 10

### 4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indazole-3-carbonitrile

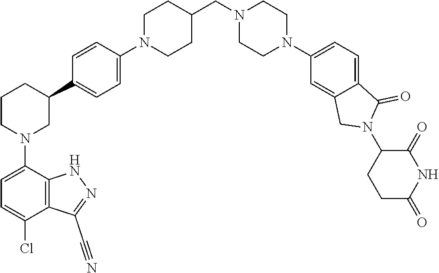 [SMILES: N#Cc1n[nH]c2c(N3CCC[C@@H](c4ccc(N5CCC(CN6CCN(c7ccc8c(c7)CN(C7CCC(=O)NC7=O)C8=O)CC6)CC5)cc4)C3)ccc(Cl)c12]

Intermediate 9g (2.0 g, 3.46 mmol) and intermediate 1i (1.48 g, 3.46 mmol) were heated in formic acid (20 mL) at 40° C. for 2 h. The reaction mixture was cooled, evaporated to dryness and the residue was stirred in NMP (20 mL) at RT for 16 h. The reaction mixture was poured into NaHCO₃ solution (300 mL) and the precipitate was collected by filtration, washed with water (100 mL) and dried under vacuum to afford crude product which was purified by flash silica chromatography, elution gradient 0 to 6% DCM in EtOH. Pure fractions were evaporated to dryness to afford product which was slurried in MeCN (50 mL) and filtered under vacuum to give the title compound (1.17 g, 44.5%) as a yellow solid. ¹H NMR δ 1.12-1.27 (2H, m), 1.49-1.59 (1H, m), 1.59-1.71 (1H, m), 1.73-2.01 (6H, m), 2.19 (2H, d), 2.28-2.43 (1H, m), 2.49 (4H, dd), 2.54-2.78 (5H, m), 2.83-3.04 (2H, m), 3.27 (4H, t), 3.46 (2H, t), 3.60 (2H, d), 4.16-4.37 (2H, m), 5.05 (1H, dd), 6.88 (3H, dd), 7.04 (2H, d), 7.15 (2H, d), 7.25 (1H, d), 7.52 (1H, d), 10.95 (1H, s), 14.62 (1H, s); m/z: ES⁺ [M+H]⁺=760.0.

### Example 11

### Intermediate 11a

### Benzyl (3S)-3-{4-[4-(dibutoxymethyl)piperidin-1-yl]phenyl}piperidine-1-carboxylate

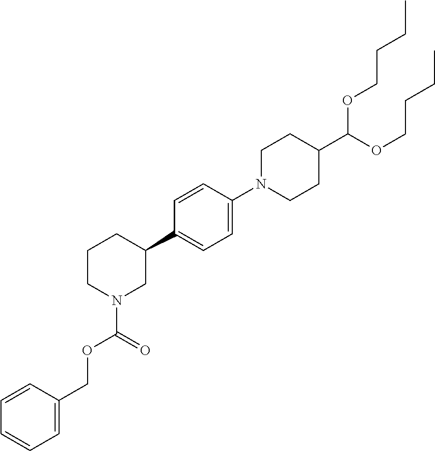 [SMILES: CCCCOC(OCCCC)C1CCN(c2ccc([C@@H]3CCCN(C(=O)OCc4ccccc4)C3)cc2)CC1]

Pd(t-Bu₃P)₂ (0.55 g, 1.07 mmol) was added to intermediate 9d (4.0 g, 10.69 mmol), 4-(dibutoxymethyl)piperidine (2.6 g, 10.69 mmol), Cs₂CO₃ (6.96 g, 21.37 mmol) and tri-tert-butylphosphonium tetrafluoroborate (0.310 g, 1.07 mmol) in 1,4-dioxane (50 mL) under nitrogen. The resulting mixture was stirred at 100° C. for 2 h. The mixture was then cooled to RT and the solvent evaporated to afford crude product. The crude product was purified by flash silica chromatography, elution gradient 0 to 40% EtOAc in Et₂O to give the title compound (2.3 g, 40.1%) as a yellow oil. ¹H NMR δ 0.89 (6H, t), 1.24-1.55 (12H, m), 1.57-1.91 (6H, m), 2.59 (1H, s), 2.82 (2H, s), 3.32-3.46 (3H, m), 3.49-3.71 (4H, m), 4.01 (2H, t), 4.19 (1H, d), 5.09 (2H, s), 6.86 (2H, d), 7.07 (2H, d), 7.35 (5H, q); m/z: (ES⁺), [M+H]⁺=537.5.

### Intermediate 11b

### 4-(Dibutoxymethyl)-1-{4-[(3S)-piperidin-3-yl]phenyl}piperidine

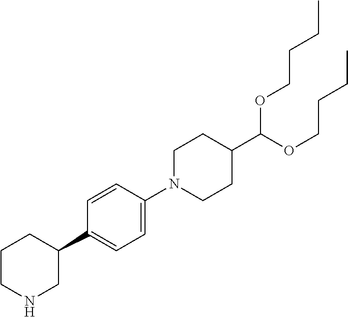 [SMILES: CCCCOC(OCCCC)C1CCN(c2ccc([C@@H]3CCCNC3)cc2)CC1]

Pd/C (10%, 0.912 g, 0.86 mmol) was added to benzyl (3S)-3-{4-[4-(dibutoxymethyl)piperidin-1-yl]phenyl}piperidine-1-carboxylate (2.3 g, 4.28 mmol) in EtOAc:MeOH (5:1, 30 mL) at RT under an atmosphere of hydrogen for 3 h. The mixture was filtered through Celite® and evaporated to dryness to give the title compound (1.6 g, 93%) as a white oil which was used without further purification. ¹H NMR δ 0.89 (6H, t), 1.24-1.42 (4H, m), 1.43-1.57 (5H, m), 1.59-1.87 (4H, m), 2.43 (4H, d), 2.57 (1H, s), 2.91 (2H, d), 3.22-3.47 (7H, m), 3.49-3.69 (4H, m), 4.19 (1H, d), 6.84 (2H, d), 7.04 (2H, d); m/z: (ES⁺), [M+H]⁺=403.1.

### Intermediate tic

### 4-Chloro-7-[(3S)-3-{4-[4-(dibutoxymethyl)piperidin-1-yl]phenyl}piperidin-1-yl]-1H-indole-3-carbonitrile

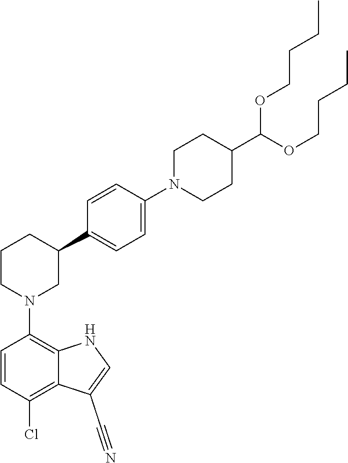 [SMILES: CCCCOC(OCCCC)C1CCN(c2ccc([C@@H]3CCCN(c4ccc(Cl)c5c(C#N)c[nH]c45)C3)cc2)CC1]

LHMDS (1M in THF, 10.43 mL, 10.43 mmol) was added to 4-(dibutoxymethyl)-1-{4-[(3S)-piperidin-3-yl]phenyl}piperidine (1.2 g, 2.98 mmol), intermediate 1b (0.838 g, 3.28 mmol) and Pd-PEPPSI-IHept^{C1} (0.145 g, 0.15 mmol) in THF (5.0 mL). The resulting mixture was stirred at 60° C. for 2 h. The reaction mixture was cooled to RT, quenched with NH₄Cl solution (10 mL), extracted with EtOAc (2×20 mL), dried over Na₂SO₄, filtered and evaporated to afford to crude product. The crude product was purified by flash silica chromatography, elution gradient 0 to 30% EtOAc in Et₂O to give the title compound (1.4 g, 81%) as a brown solid. ¹H NMR δ 0.89 (6H, t), 1.27-1.4 (6H, m), 1.43-1.54 (5H, m), 1.72 (4H, d), 1.79-1.98 (3H, m), 2.53-2.8 (4H, m), 2.98 (1H, d), 3.36-3.43 (3H, m), 3.49-3.7 (4H, m), 4.18 (1H, d), 6.8-6.9 (3H, m), 7.09-7.2 (3H, m), 8.31 (1H, d), 12.28 (1H, d); m/z: (ES⁺), [M+H]⁺=577.3.

### Example 11

### 4-Chloro-7-[(3S)-3-{4-[4-({4-[4-(2,4-dioxo-1,3-diazinan-1-yl)-1H-indol-1-yl]piperidin-1-yl}methyl)piperidin-1-ylphenyl}piperidin-1-yl]-1H-indole-3-carbonitrile

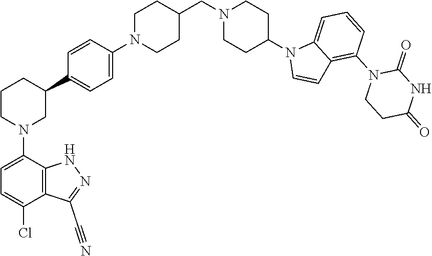 [SMILES: N#Cc1n[nH]c2c(N3CCC[C@@H](c4ccc(N5CCC(CN6CCC(n7ccc8c(N9CCC(=O)NC9=O)cccc87)CC6)CC5)cc4)C3)ccc(Cl)c12]

4-Chloro-7-[(3S)-3-{4-[4-(dibutoxymethyl)piperidin-1-yl]phenyl}piperidin-1-yl]-1H-indole-3-carbonitrile (1.2 g, 2.08 mmol) was heated in formic acid (16.0 ml, 417.16 mmol) at 40° C. for 1 h. The mixture was cooled and evaporated and diluted in NMP (16.0 mL). Intermediate 5f (1.01 g, 2.08 mmol) and sodium acetate (0.205 g, 2.49 mmol) was added and the resulting mixture was stirred at 40° C. for 2 h. The reaction mixture was then poured into Na₂CO₃ solution (150 mL) and the precipitate was collected by filtration, washed with water (50 mL) and dried under vacuum to afford crude product. The crude product was purified by flash silica chromatography, elution gradient 0 to 10% DCM in EtOH to afford product which was slurried in MeCN (50 mL) and filtered under vacuum to give the title compound (1.16 g, 75%) as a white solid. ¹H NMR δ 1.21 (2H, m), 1.46-1.72 (2H, m), 1.82 (3H, dd), 1.87-2.05 (6H, m), 2.17 (4H, dd), 2.63 (4H, dq), 2.76 (2H, t), 2.97 (3H, d), 3.30 (1H, d), 3.36 (1H, s), 3.61 (2H, d), 3.77 (2H, t), 4.36 (1H, tt), 6.41 (1H, d), 6.84 (3H, dd), 6.96 (1H, d), 7.14 (4H, dd), 7.44-7.61 (2H, m), 8.31 (1H, s), 10.34 (1H, s), 12.28 (1H, s); m/z: ES⁺ [M+H]⁺=743.3.

### Example 12

### 4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile

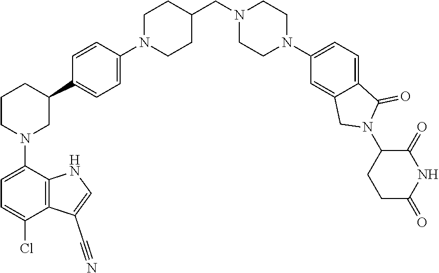 [SMILES: N#Cc1c[nH]c2c(N3CCC[C@@H](c4ccc(N5CCC(CN6CCN(c7ccc8c(c7)CN(C7CCC(=O)NC7=O)C8=O)CC6)CC5)cc4)C3)ccc(Cl)c12]

Intermediate 11c (2.3 g, 3.98 mmol) and intermediate 1i (1.7 g, 3.98 mmol) were heated in formic acid (20 mL) at 40° C. for 1 h. The reaction was then cooled and evaporated to afford a residue which was diluted with NMP (20 mL). The resulting mixture was stirred at 40° C. for 16 h and then poured into Na₂CO₃ solution (300 mL). The precipitate was collected by filtration, washed with water (50 mL) and dried under vacuum to afford crude product. The crude product was purified by flash silica chromatography, elution gradient 0 to 6% DCM in EtOH to afford product which was slurried in MeCN and filtered under vacuum to give the title compound (0.93 g, 30.7%) as a white solid. ¹H NMR δ 1.14-1.28 (2H, m), 1.46-1.61 (1H, m), 1.68 (1H, d), 1.74-1.87 (3H, m), 1.88-2 (3H, m), 2.20 (2H, d), 2.28-2.44 (1H, m), 2.45-2.5 (3H, m), 2.51 (1H, s), 2.54-2.73 (5H, m), 2.83-3.03 (2H, m), 3.24-3.31 (4H, m), 3.35 (2H, d), 3.61 (2H, d), 4.21 (1H, d), 4.33 (1H, d), 5.05 (1H, dd), 6.84 (3H, dd), 7.05 (2H, d), 7.14 (3H, dd), 7.52 (1H, d), 8.31 (1H, s), 10.94 (1H, s), 12.27 (1H, s); m/z: ES⁺ [M+H]⁺=759.5.

### Example 13

### Intermediate 13a

### tert-Butyl 4-[(2-amino-4-bromophenyl)ethynyl]piperidine-1-carboxylate

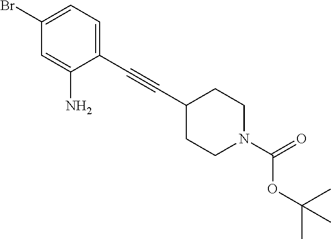 [SMILES: CC(C)(C)OC(=O)N1CCC(C#Cc2ccc(Br)cc2N)CC1]

PdCl₂(PPh₃)₂ (3.36 g, 4.78 mmol) was added in one portion to tert-butyl 4-ethynylpiperidine-1-carboxylate (10.01 g, 47.83 mmol), 5-bromo-2-iodoaniline (14.25 g, 47.83 mmol), copper(I) iodide (1.36 g, 7.17 mmol) and triethylamine (19.97 ml, 143.49 mmol) in DMF (145 mL) at RT under nitrogen. The resulting dark brown solution was stirred at RT for 18 h. The reaction mixture was diluted with water (150 mL) and the product extracted into MTBE (150 mL). The organic layer was washed with water (150 mL), NaCl solution (50 mL), dried with MgSO₄ and evaporated to afford crude product. The crude product was purified by column chromatography, elution gradient 5 to 20% heptane:EtOAc to give the title compound (24.14 g, 55%) as an orange oil. ¹H NMR δ 1.46 (9H, s), 1.68 (2H, m), 1.88 (2H, m), 2.83 (1H, m), 3.18-3.26 (2H, m), 3.76 (2H, m), 4.18 (2H, s), 6.78 (1H, m), 6.84 (1H, m), 7.08 (1H, m); m/z: ES⁺ [M+H]⁺=379.1.

### Intermediate 13b

### tert-Butyl 4-(6-bromo-1-methyl-1H-indol-2-yl)piperidine-1-carboxylate

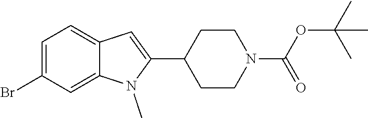 [SMILES: Cn1c(C2CCN(C(=O)OC(C)(C)C)CC2)cc2ccc(Br)cc21]

Potassium tert-butoxide (13.30 g, 118.48 mmol) was added portion wise to tert-butyl 4-[(2-amino-4-bromophenyl)ethynyl]piperidine-1-carboxylate (21.4 g, 39.49 mmol) in NMP (200 mL) and stirred at RT for 3 h. Iodomethane (7.38 ml, 118.48 mmol) was added portion wise and stirred at RT for a further 1 h. The reaction mixture was then quenched with NH₄Cl solution (100 mL) and water (100 mL). The product was extracted with 2-MeTHF (2×200 mL) and the combined organic layers were washed with water (2×100 mL), NaCl solution (100 mL), dried over MgSO₄ and evaporated to afford crude product as a brown oil. The crude product was purified by flash silica chromatography, elution gradient 0 to 50% EtOAc in heptane. Pure fractions were evaporated to afford a solid which was triturated with heptane (50 mL). The solid was collected by filtration and dried in a vacuum oven at 45° C. to give the title compound (13.74 g, 88%) as a white solid. ¹H NMR δ 1.51 (9H, s), 1.68 (2H, qd), 1.99 (2H, d), 2.79-2.96 (3H, m), 3.70 (3H, s), 4.28 (2H, m), 6.24 (1H, s), 7.19 (1H, m), 7.39-7.44 (1H, m), 7.45 (1H, m); m/z: ES⁺ [M+H]⁺=393.3.

### Intermediate 13c

### tert-Butyl 4-[6-(2,4-dioxo-1,3-diazinan-1-yl)-1-methyl-1H-indol-2-yl]piperidine-1-carboxylate

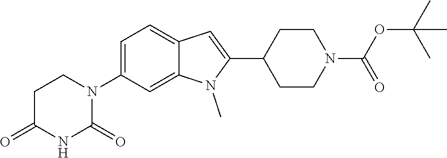 [SMILES: Cn1c(C2CCN(C(=O)OC(C)(C)C)CC2)cc2ccc(N3CCC(=O)NC3=O)cc21]

tert-Butyl 4-(6-bromo-1-methyl-1H-indol-2-yl)piperidine-1-carboxylate (10.82 g, 27.51 mmol) was dissolved in 1,4-dioxane (180 mL) followed by addition of hexahydropyrimidine-2,4-dione (9.42 g, 82.53 mmol), K₂CO₃ (11.41 g, 82.53 mmol) and tert-butylBrettPhos (1.33 g, 2.75 mmol). The mixture was degassed with nitrogen then tert-butylBrettPhos Pd G3 (2.350 g, 2.75 mmol) was added and the mixture stirred at 100° C. for 42 h. The reaction mixture was cooled, water (180 mL) and 2-MeTHF (180 mL) were added and the layers separated. The organic layer was washed with water (180 mL), NaCl solution (50 mL), dried with MgSO₄, filtered and evaporated to afford crude product as a dark brown solid. The solid was slurried in EtOAc (30 mL) and the resulting brown suspension was filtered and washed with a EtOAc (10 mL) to give the title compound (6.59 g, 56.2%) as a pale brown solid. ¹H NMR δ 1.43 (11H, m), 1.94 (2H, dm, 2.73 (2H, t), 2.82-3.07 (3H, m), 3.70 (3H, s), 3.79 (2H, m), 4.06 (2H, dd), 6.24 (1H, s), 6.94 (1H, dd), 7.37 (1H, d), 7.43 (1H, d), 10.26 (1H, s); m/z: ES⁺ [M-tert-Bu+H]⁺=371.1.

### Example 13

### 4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indole-3-carbonitrile

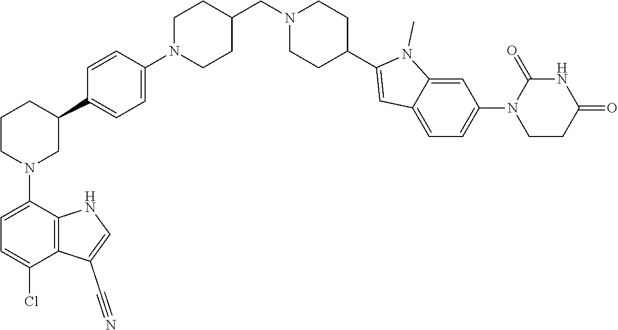 [SMILES: Cn1c(C2CCN(CC3CCN(c4ccc([C@@H]5CCCN(c6ccc(Cl)c7c(C#N)c[nH]c67)C5)cc4)CC3)CC2)cc2ccc(N3CCC(=O)NC3=O)cc21]

tert-Butyl 4-[6-(2,4-dioxo-1,3-diazinan-1-yl)-1-methyl-1H-indol-2-yl]piperidine-1-carboxylate (1.55 g, 3.64 mmol) and intermediate 11c (2.1 g, 3.64 mmol) were heated in formic acid (20 mL, 3.64 mmol) at 40° C. for 2 h. The reaction was then cooled and evaporated to afford a residue which was diluted with NMP (20 mL). The resulting mixture was stirred at 40° C. for 16 h. The reaction mixture was then poured into Na₂CO₃ solution (150 mL) and the precipitate was collected by filtration, washed with water (50 mL) and dried under vacuum to afford crude product. The crude product was purified by flash silica chromatography, elution gradient 0 to 10% DCM in EtOH to afford product which was slurried in MeCN and filtered under vacuum to give the title compound (1.10 g, 39.9%) as a pale yellow solid. ¹H NMR δ 1.12-1.28 (2H, m), 1.47-1.58 (1H, m), 1.58-1.73 (3H, m), 1.73-1.88 (3H, m), 1.88-1.99 (4H, m), 2.04 (2H, t), 2.18 (2H, d), 2.53-2.69 (4H, m), 2.73 (3H, t), 2.85-3.05 (3H, m), 3.30 (1H, d), 3.36 (1H, d), 3.61 (2H, d), 3.68 (3H, s), 3.78 (2H, t), 6.22 (1H, s), 6.79-6.9 (3H, m), 6.93 (1H, dd), 7.14 (3H, dd), 7.36 (1H, d), 7.42 (1H, d), 8.31 (1H, s), 10.30 (1H, s), 12.28 (1H, s); m z; ES⁺ [M+H]⁺=757.4.

### Example 14

### Example 14a

### 1-[1-Methyl-2-(piperidin-4-yl)-1H-indol-6-yl]-1,3-diazinane-2,4-dione tosylate

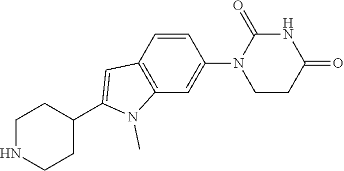 [SMILES: Cn1c(C2CCNCC2)cc2ccc(N3CCC(=O)NC3=O)cc21]

A mixture of intermediate 13c (1.6 g, 3.75 mmol) and 4-methylbenzenesulfonic acid hydrate (0.856 g, 4.50 mmol) in MeCN (16.0 mL) was stirred at 70° C. for 2.5 h. The reaction mixture was cooled to RT, diluted with MTBE (16 mL) and stirred for 15 mins. The solid was filtered, washing the solid with excess MTBE. The solid was dried under vacuum at 40° C. to give the title compound (1.65 g, 88%) as a cream solid. ¹H NMR δ 1.71-1.85 (2H, m), 2.12 (2H, m), 2.29 (3H, m), 2.74 (2H, t), 3.03-3.24 (3H, m), 3.41 (1H, m), 3.71 (3H, s), 3.79 (2H, t), 6.26 (1H, s), 7.45-7.52 (3H, m); m/z: ES⁺ [M+H]⁺=327.3.

### Example 14

### 4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile

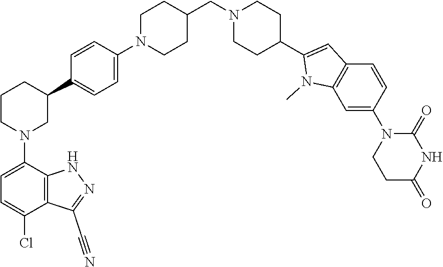 [SMILES: Cn1c(C2CCN(CC3CCN(c4ccc([C@@H]5CCCN(c6ccc(Cl)c7c(C#N)n[nH]c67)C5)cc4)CC3)CC2)cc2ccc(N3CCC(=O)NC3=O)cc21]

A suspension of intermediate 9h (1.38 g, 3.08 mmol) and 1-[1-methyl-2-(piperidin-4-yl)-1H-indol-6-yl]-1,3-diazinane-2,4-dione TsOH (1.61 g, 3.23 mmol) in NMP (14.0 mL) was stirred at RT for 18 h. Sodium triacetoxyborohydride (0.78 g, 3.70 mmol) was added and the solution was stirred at RT for 2 h. The reaction mixture was quenched with water (40 mL) and the suspension was stirred at RT for 30 mins. The solid was collected by filtration and was washed with water. The solid was dissolved in DCM:IPA (3:1, 150 mL) and washed with NaHCO₃ solution (150 mL), NaCl solution (100 mL), dried over a phase separating cartridge and evaporated to dryness to afford crude product. The crude product was purified by flash silica chromatography, elution gradient 0 to 100% EtOAc in heptane followed by 0 to 10% MeOH in DCM to afford the desired product which was slurried in MeCN (30 mL) at 80° C. for 3 h and then left to cool to RT for 18 h. The solid was filtered under vacuum to give the title compound (1.27 g, 54.4%) as a pale pink solid. ¹H NMR δ 1.16-1.28 (2H, m), 1.53-1.73 (4H, m), 1.76-1.98 (7H, m), 2.07-2.17 (2H, m), 2.25 (2H, d), 2.56-2.65 (2H, m), 2.66-2.84 (5H, m), 2.93-3.04 (3H, m), 3.47-3.57 (2H, m), 3.63 (2H, d), 3.68 (3H, s), 3.78 (2H, t), 6.23 (1H, s), 6.85-6.91 (3H, m), 6.93 (1H, dd), 7.18 (2H, d), 7.24 (1H, d), 7.36 (1H, s), 7.42 (1H, d), 10.27 (1H, s), 14.43 (1H, s); m/z: ES⁺ [M+H]⁺=758.4.

### Example 15

### 7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indole-3-carbonitrile

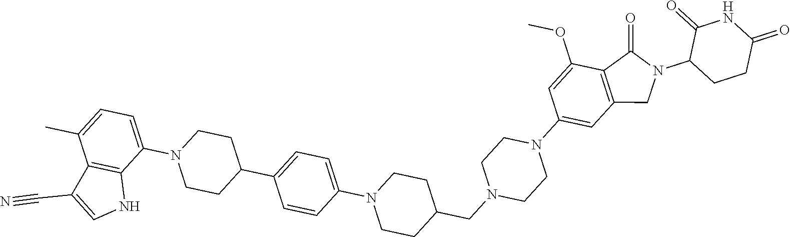 [SMILES: COc1cc(N2CCN(CC3CCN(c4ccc(C5CCN(c6ccc(C)c7c(C#N)c[nH]c67)CC5)cc4)CC3)CC2)cc2c1C(=O)N(C1CCC(=O)NC1=O)C2]

Intermediate 8b was reacted with Intermediate 7c using the general synthetic method illustrated by Example 7 after purification by HPLC (Column A, Eluent A) to give the title compound (formate salt) (31 mg, 18%) as a white solid. ¹H NMR δ 1.20-1.27 (3H, m), 1.68 (2H, s), 1.82 (4H, d), 1.95 (3H, s), 2.23 (2H, s), 2.31 (1H, d), 2.51 (4H, s), 2.56-2.68 (7H, m), 2.74 (2H, t), 2.88 (1H, q), 3.30 (2H, s), 3.38 (2H, d), 3.64 (2H, d), 3.84 (3H, s), 4.05-4.27 (2H, m), 4.96 (1H, dd), 6.49 (1H, s), 6.61 (1H, s), 6.77 (1H, d), 6.89 (3H, dd), 7.13 (2H, d), 8.16 (1H, d), 10.91 (1H, s), 11.90 (1H, s); m/z: ES⁺ [M+H]⁺=769.4.

### Examples 16 and 17

The enantiomers of Example 7 were separated by SFC purification on a Sepiatec 100 SFC using SFC conditions Regis (R,R) Whelk-01, 21.1×250 mm, 5 micron, 50% MeOH/MeCN 3:7/50% scCO2 at a flow rate of 60 m/min, 40° C. to give, in order of elution, example 16 (isomer 1, 12 mg, 28.6%) and example 17 (isomer 2, 13 mg, 31.0%) as white solids.

### Example 16

### 4-Chloro-7-[4-(4-{4-[(4-{2-[(3R*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile [Absolute Stereochemistry not Yet Confirmed]

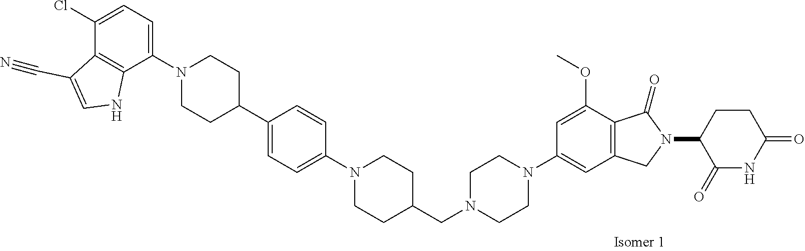 [SMILES: COc1cc(N2CCN(CC3CCN(c4ccc(C5CCN(c6ccc(Cl)c7c(C#N)c[nH]c67)CC5)cc4)CC3)CC2)cc2c1C(=O)N([C@H]1CCC(=O)NC1=O)C2]

¹H NMR δ 1.26 (2H, s), 1.70 (1H, s), 1.84 (4H, s), 1.97 (3H, d), 2.24 (3H, d), 2.54-2.65 (4H, m), 2.72-2.85 (2H, m), 2.88 (1H, d), 3.43 (2H, d), 3.65 (2H, d), 3.84 (3H, s), 4.11 (1H, d), 4.24 (1H, d), 4.97 (1H, dd), 6.50 (1H, s), 6.62 (1H, s), 6.87 (1H, d), 6.91 (2H, d), 7.15 (3H, dd), 8.32 (1H, s), 10.90 (1H, s), 12.26 (1H, s); m/z: ES⁺ [M+H]⁺=789.6; >99% cc.

### Example 17

### 4-Chloro-7-[4-(4-{4-[(4-{2-[(3S*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile [Absolute Stereochemistry not Yet Confirmed]

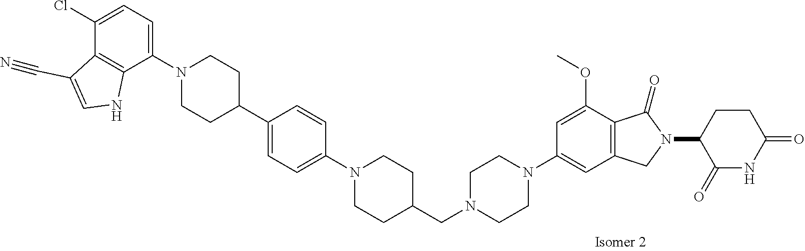 [SMILES: COc1cc(N2CCN(CC3CCN(c4ccc(C5CCN(c6ccc(Cl)c7c(C#N)c[nH]c67)CC5)cc4)CC3)CC2)cc2c1C(=O)N([C@H]1CCC(=O)NC1=O)C2]

¹H NMR δ 1.26 (2H, s), 1.70 (1H, s), 1.84 (4H, s), 1.98 (3H, d), 2.24 (2H, d), 2.31 (1H, s), 2.62 (4H, d), 2.73-2.83 (2H, m), 2.83-2.95 (1H, m), 3.43 (2H, d), 3.65 (2H, d), 3.84 (3H, s), 4.11 (1H, d), 4.24 (1H, d), 4.97 (1H, dd), 6.50 (1H, s), 6.62 (1H, s), 6.89 (3H, dd), 7.15 (3H, dd), 8.32 (1H, s), 10.90 (1H, s), 12.26 (1H, s); m/z: ES⁺ [M+H]⁺=789.6; >99% ee.

### Examples 18 and 19

The enantiomers of Example 1 were separated by SFC purification on a Sepiatec 100 SFC using SFC conditions Regis (R,R) Whelk-01, 21.1×250 mm, 5 micron, 50% MeOH/MeCN 3:7/50% scCO2 at a flow rate of 60 m/min, 40° C. to give, in order of elution, example 18 (isomer 1, 410 mg, 20.8%) and example 19 (isomer 2, 400 mg, 20.3%) as white solids.

### Example 18

### 4-Chloro-7-[4-(4-{4-[(4-{2-[(3R*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile [Absolute Stereochemistry not Yet Confirmed]

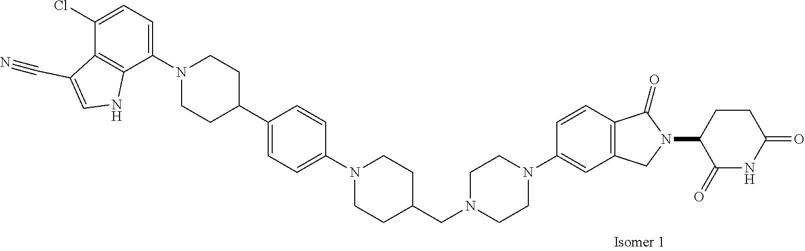 [SMILES: N#Cc1c[nH]c2c(N3CCC(c4ccc(N5CCC(CN6CCN(c7ccc8c(c7)CN([C@H]7CCC(=O)NC7=O)C8=O)CC6)CC5)cc4)CC3)ccc(Cl)c12]

¹H NMR δ 1.25 (3H, s), 1.70 (1H, s), 1.84 (4H, s), 1.97 (4H, d), 2.24 (2H, d), 2.35-2.43 (2H, m), 2.55-2.65 (3H, m), 2.78 (2H, t), 2.84-2.97 (1H, m), 3.43 (2H, d), 3.65 (2H, d), 4.22 (1H, d), 4.34 (1H, d), 5.05 (1H, dd), 6.86 (1H, d), 6.91 (2H, d), 7.07 (2H, d), 7.11-7.19 (3H, m), 7.53 (1H, d), 8.31 (1H, s), 10.94 (1H, s), 12.26 (1H, s); m/z: ES⁺ [M+H]⁺=759.7; >99% ee.

### Example 19

### 4-Chloro-7-[4-(4-{4-[(4-{2-[(3S*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile [Absolute Stereochemistry not Yet Confirmed]

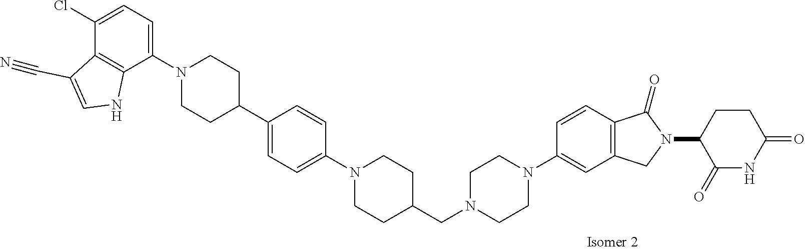 [SMILES: N#Cc1c[nH]c2c(N3CCC(c4ccc(N5CCC(CN6CCN(c7ccc8c(c7)CN([C@H]7CCC(=O)NC7=O)C8=O)CC6)CC5)cc4)CC3)ccc(Cl)c12]

¹H NMR δ 1.16-1.32 (2H, m), 1.70 (1H, s), 1.78-1.9 (4H, m), 1.97 (3H, d), 2.24 (2H, d), 2.37 (2H, dd), 2.55-2.59 (1H, m), 2.62 (2H, d), 2.78 (2H, t), 2.84-2.96 (1H, m), 3.43 (2H, d), 3.65 (2H, d), 4.22 (1H, d), 4.34 (1H, d), 5.05 (1H, dd), 6.87 (1H, d), 6.91 (2H, d), 7.07 (2H, d), 7.15 (3H, dd), 7.53 (1H, d), 8.32 (1H, s), 10.94 (1H, s), 12.26 (1H, s); m/z: ES⁺ [M+H]⁺=759.7; >99% ee.

### Example 20

### 7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile

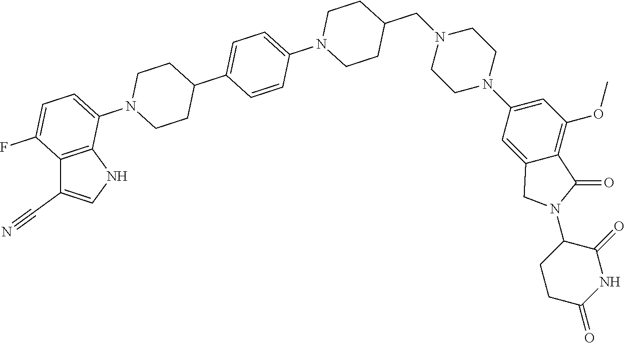 [SMILES: COc1cc(N2CCN(CC3CCN(c4ccc(C5CCN(c6ccc(F)c7c(C#N)c[nH]c67)CC5)cc4)CC3)CC2)cc2c1C(=O)N(C1CCC(=O)NC1=O)C2]

Intermediate 2c was reacted with Intermediate 7c using the general synthetic method illustrated by Example 7 after purification by HPLC (Column A, Eluent A) to give the title compound in the form of a formate salt (20 mg, 14%) as a white solid. ¹H NMR δ 1.24 (2H, d), 1.69 (1H, s), 1.85 (4H, s), 1.88-2.05 (3H, m), 2.23 (2H, d), 2.25-2.37 (1H, m), 2.56-2.74 (4H, m), 2.78 (2H, d), 2.88 (1H, d), 3.30 (8H, d), 3.39 (1H, s), 3.65 (3H, d), 3.84 (3H, s), 4.03-4.29 (2H, m), 4.91-5.03 (1H, m), 6.50 (1H, s), 6.62 (1H, s), 6.77-6.87 (1H, m), 6.87-7.00 (3H, m), 7.14 (2H, d), 8.28 (1H, s), 10.92 (1H, s), 12.26 (1H, s); m/z: ES⁺ [M+H]⁺=773.0.

### Example 21

### Intermediate 21a

### Benzyl (3R)-3-(4-bromophenyl)piperidine-1-carboxylate

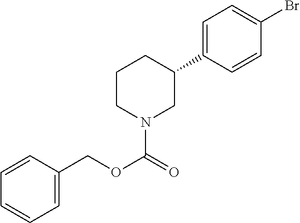 [SMILES: O=C(OCc1ccccc1)N1CCC[C@H](c2ccc(Br)cc2)C1]

(R)-3-(4-Bromophenyl)piperidine oxalate was reacted with benzyl carbonochloridate using the general synthetic method illustrated by example 1c to give the title compound (1.0 g, 64.2%) as a colourless oil. ¹H NMR δ 1.4-1.54 (1H, m), 1.58-1.67 (1H, m), 1.67-1.77 (1H, m), 1.87 (1H, dd), 2.59-2.71 (1H, m), 2.87 (2H, s), 3.9-4.12 (2H, m), 5.09 (2H, d), 7.24 (2H, d), 7.34 (5H, q), 7.47-7.53 (2H, m); m/z: ES⁺ [M+H]⁺=374.0.

### Intermediate 21b

### Benzyl (3R)-3-{4-[4-(dibutoxymethyl)piperidin-1-yl]phenyl}piperidine-1-carboxylate

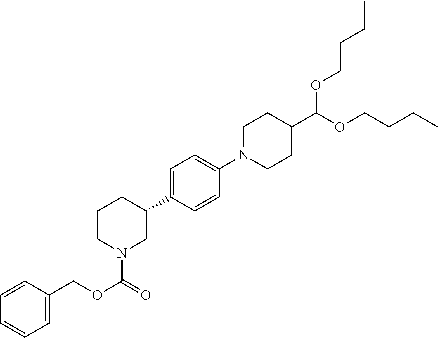 [SMILES: CCCCOC(OCCCC)C1CCN(c2ccc([C@H]3CCCN(C(=O)OCc4ccccc4)C3)cc2)CC1]

Benzyl (3R)-3-(4-bromophenyl)piperidine-1-carboxylate was reacted with 4-(dibutoxymethyl)piperidine using the general synthetic method illustrated by example 11a to give the title compound (462 mg, 32.1%) as a yellow oil. ¹H NMR δ 0.88 (6H, t), 1.24-1.37 (7H, m), 1.41-1.54 (6H, m), 1.54-1.76 (6H, m), 1.84 (1H, d), 2.52-2.59 (2H, m), 3.35-3.45 (2H, m), 3.51-3.6 (2H, m), 3.63 (2H, d), 4.01 (2H, t), 4.18 (1H, d), 5.08 (2H, s), 6.85 (2H, d), 7.07 (2H, d), 7.27-7.47 (5H, m); m/z: ES⁺ [M+H]⁺=537.2.

### Intermediate 21c

### 4-(Dibutoxymethyl)-1-{4-[(3R)-piperidin-3-yl]phenyl}piperidine

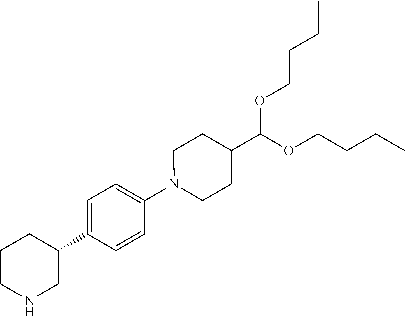 [SMILES: CCCCOC(OCCCC)C1CCN(c2ccc([C@H]3CCCNC3)cc2)CC1]

Benzyl (3R)-3-{4-[4-(dibutoxymethyl)piperidin-1-yl]phenyl}piperidine-1-carboxylate was reacted with Pd/C (10%) and hydrogen gas using the general synthetic method illustrated by example 11b to give the title compound (300 mg, 87.0%) as a colourless oil. ¹H NMR δ 0.88 (6H, t), 1.23-1.4 (7H, m), 1.41-1.55 (6H, m), 1.58-1.89 (5H, m), 2.42 (3H, q), 2.54 (2H, d), 2.90 (2H, d), 3.34-3.43 (2H, m), 3.52-3.57 (2H, m), 3.58-3.65 (2H, m), 4.18 (1H, d), 6.76-6.9 (2H, m), 6.99-7.1 (2H, m); m/z: ES⁺ [M+H]⁺=403.2.

### Intermediate 21d

### 4-Chloro-7-[(3R)-3-{4-[4-(dibutoxymethyl)piperidin-1-yl]phenyl}piperidin-1-yl]-1H-indole-3-carbonitrile

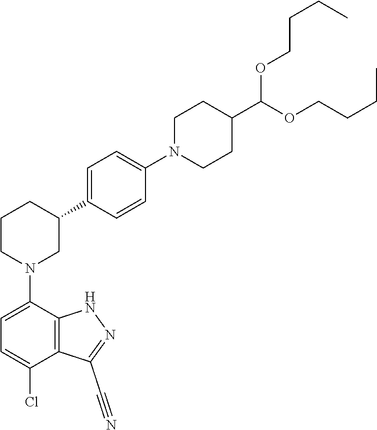 [SMILES: CCCCOC(OCCCC)C1CCN(c2ccc([C@H]3CCCN(c4ccc(Cl)c5c(C#N)n[nH]c45)C3)cc2)CC1]

4-(Dibutoxymethyl)-1-{4-[(3R)-piperidin-3-yl]phenyl}piperidine was reacted with intermediate 1b using the general synthetic method illustrated by example 11c to give the title compound (60 mg, 27.9%) as a brown solid. ¹H NMR δ 0.89 (6H, t), 1.3-1.42 (5H, m), 1.48 (5H, q), 1.72 (3H, d), 1.8-2.06 (4H, m), 2.66 (3H, d), 3.00 (1H, s), 3.41 (5H, s), 3.5-3.72 (4H, m), 4.19 (1H, d), 6.86 (3H, t), 7.15 (3H, t), 8.31 (1H, d), 12.28 (1H, s); m/z: ES⁺ [M+H]⁺=577.2.

### Example 21

### 4-Chloro-7-[(3R)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile

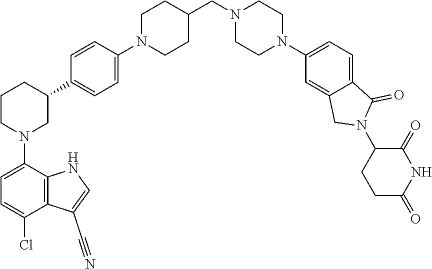 [SMILES: N#Cc1c[nH]c2c(N3CCC[C@H](c4ccc(N5CCC(CN6CCN(c7ccc8c(c7)CN(C7CCC(=O)NC7=O)C8=O)CC6)CC5)cc4)C3)ccc(Cl)c12]

Intermediate 21d was reacted with Intermediate 1i using the general synthetic method illustrated by Example 7 after purification by HPLC (Column A, Eluent A) gave the title compound in the form of a formate salt (30 mg, 28%) as a white solid. ¹H NMR δ 1.21 (2, q), 1.48-1.62 (1H, m), 1.68 (1H, s), 1.72-2.03 (7H, m), 2.21 (2H, d), 2.28-2.48 (2H, m), 2.5-2.75 (6H, m), 2.84-3.07 (2H, m), 3.28 (5H, s), 3.35 (2H, s), 3.62 (2H, d), 4.20 (1H, d), 4.33 (1H, d), 5.05 (1H, dd), 6.85 (3H, dd), 7.07 (2H, d), 7.15 (3H, dd), 7.52 (1H, d), 8.31 (1H, d), 10.95 (1H, s), 12.28 (1H, d); m/z: ES⁺ [M+H]⁺=759.4.

### Example 22

### Intermediate 22a

### tert-Butyl 4-(4-{1-[(benzyloxy)carbonyl]piperidin-4-yl}phenyl)piperazine-1-carboxylate

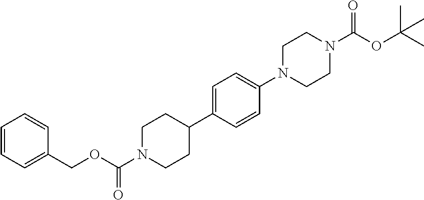 [SMILES: CC(C)(C)OC(=O)N1CCN(c2ccc(C3CCN(C(=O)OCc4ccccc4)CC3)cc2)CC1]

Pd(t-Bu₃P)₂ (0.137 g, 0.27 mmol) was added to tri-tert-butylphosphonium tetrafluoroborate (0.078 g, 0.27 mmol), Cs₂CO₃ (1.741 g, 5.34 mmol), tert-butyl piperazine-1-carboxylate (0.498 g, 2.67 mmol) and intermediate 1c (1.00 g, 2.67 mmol) in 1,4-dioxane (10 mL) under nitrogen. The resulting mixture was stirred at 100° C. for 19 h. The reaction was then cooled to RT and the solvent was evaporated to afford crude product. The crude product was purified by flash silica chromatography, elution gradient 0 to 40% EtOAc in Et₂O to give the title compound (1.0 g, 78%) as a yellow solid. m/z: ES⁺ [M+H]⁺=480.4.

### Intermediate 22b

### tert-Butyl 4-[4-(piperidin-4-yl)phenyl]piperazine-1-carboxylate

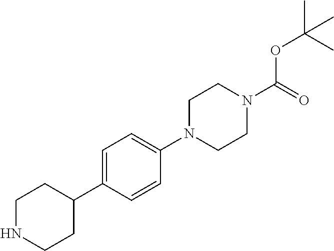 [SMILES: CC(C)(C)OC(=O)N1CCN(c2ccc(C3CCNCC3)cc2)CC1]

Pd/C (10%, 0.67 g, 0.63 mmol) and tert-butyl 4-(4-{1-[(benzyloxy)carbonyl]piperidin-4-yl}phenyl)piperazine-1-carboxylate (1.00 g, 2.08 mmol) in MeOH:EtOAc (1:5, 1.2 mL) was stirred under an atmosphere of hydrogen at RT for 2 h. The mixture was filtered through Celite® and evaporated to dryness to give the title compound which was used without further purification. m/z: ES⁺ [M+H]⁺=346.3.

### Intermediate 22c

### tert-Butyl 4-{4-[1-(3-cyano-4-fluoro-1H-indol-7-yl)piperidin-4-yl]phenyl}piperazine-1-carboxylate

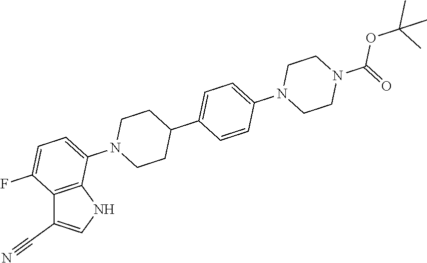 [SMILES: CC(C)(C)OC(=O)N1CCN(c2ccc(C3CCN(c4ccc(F)c5c(C#N)c[nH]c45)CC3)cc2)CC1]

(DiMeIHeptCl)Pd(cinnamyl)^{C1} (152 mg, 0.14 mmol) was added to tert-butyl 4-(4-(piperidin-4-yl)phenyl)piperazine-1-carboxylate (500 mg, 1.45 mmol), intermediate 2b (346 mg, 1.45 mmol) and Cs₂CO₃ (1.6 g, 5.07 mmol) in 1,4-dioxane (1 mL) under nitrogen and stirred at 100° C. for 3 h. The reaction was cooled to RT and the solvent was evaporated to afford crude product. The crude product was purified by flash silica chromatography, elution gradient 0 to 50% EtOAc in Et₂O to give the title compound as a yellow solid. m/z: ES⁺ [M+H]⁺=504.3.

### Intermediate 22d

### 3-{5-[4-(Dibutoxymethyl)piperidin-1-yl]-1-oxo-1,3-dihydro-2H-isoindol-2-yl}piperidine-2,6-dione

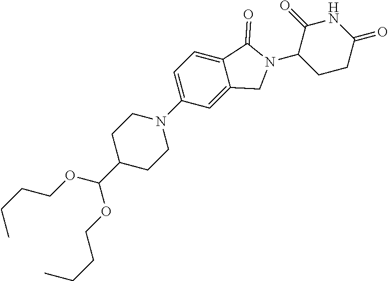 [SMILES: CCCCOC(OCCCC)C1CCN(c2ccc3c(c2)CN(C2CCC(=O)NC2=O)C3=O)CC1]

Pd-PEPPSI-IHept^{C1} (0.23 g, 0.24 mmol) was added to a degassed mixture of intermediate 1h (1.5 g, 4.64 mmol), 4-(dibutoxymethyl)piperidine (1.5 g, 6.16 mmol) and Cs₂CO₃ (4.54 g, 13.93 mmol) in 1,4-dioxane (45 mL) at RT under nitrogen. The resulting mixture was stirred at 100° C. for 3 h under nitrogen. The reaction mixture was cooled to RT, diluted with DCM (75 mL) and AcOH (10%, 50 mL). The organic layer was washed with NaHCO₃ solution (50 mL), dried with MgSO₄, filtered and evaporated to afford crude product. The crude product was triturated with EtOAc (15 mL) to give a solid which was collected by filtration, washed with EtOAc (2×5 mL), EtOAc:Et₂O (1:1; 5 mL), Et₂O (5 mL) and dried under vacuum to give the title compound (1.66 g, 73.6%) as a white solid. ¹H NMR δ 0.89 (6H, t), 1.23-1.42 (6H, m), 1.43-1.56 (4H, m), 1.67-1.85 (3H, m), 1.96 (1H, m), 2.28-2.44 (1H, m), 2.55-2.64 (1H, m), 2.72-2.84 (2H, m), 2.90 (1H, m), 3.40 (2H, dt), 3.56 (2H, dt), 3.89 (2H, d), 4.09-4.25 (2H, m), 4.32 (1H, d), 5.04 (1H, dd), 7.04 (2H, d), 7.50 (1H, d), 10.91 (1H, s); m/z: ES⁺ [M+H]⁺=486.0.

### Example 22

### 7-(4-{4-[4-({1-[2-(2,6-Dioxopiperidin-3-yl)-1-oxo-2,3-dihydro-1H-isoindol-5-yl]piperidin-4-yl}methyl)piperazin-1-ylphenyl}piperidin-1-yl)-4-fluoro-1H-indole-3-carbonitrile

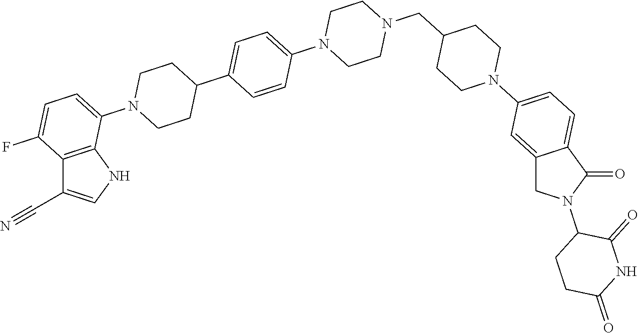 [SMILES: N#Cc1c[nH]c2c(N3CCC(c4ccc(N5CCN(CC6CCN(c7ccc8c(c7)CN(C7CCC(=O)NC7=O)C8=O)CC6)CC5)cc4)CC3)ccc(F)c12]

Intermediate 22c was reacted with intermediate 22d using the general synthetic method illustrated by Example 7 after purification by HPLC (Column A, Eluent A) gave the title compound in the form of a formate salt (25.0 mg, 14.12%) as a yellow solid. ¹H NMR δ 1.19 (2H, q), 1.82 (5H, t), 1.89-2.03 (3H, m), 2.20 (2H, d), 2.29-2.44 (1H, m), 2.54-2.62 (2H, m), 2.65-2.8 (3H, m), 2.83 (2H, t), 2.85-2.97 (1H, m), 3.06-3.13 (4H, m), 3.26-3.38 (5H, m), 3.88 (2H, d), 4.19 (1H, d), 4.32 (1H, d), 5.05 (1H, dd), 6.82 (1H, dd), 6.87-6.97 (3H, m), 7.04 (2H, d), 7.14 (2H, d), 7.50 (1H, d), 8.27 (1H, s), 10.95 (1H, s), 12.26 (1H, s); m/z: ES⁺ [M+H]⁺=743.5.

### Example 23

### Intermediate 23a

### 7-[(3S)-3-{4-[4-(Dibutoxymethyl)piperidin-1-yl]phenyl}piperidin-1-yl]-4-fluoro-1H-indole-3-carbonitrile

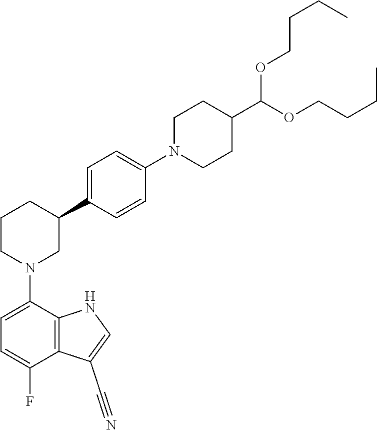 [SMILES: CCCCOC(OCCCC)C1CCN(c2ccc([C@@H]3CCCN(c4ccc(F)c5c(C#N)c[nH]c45)C3)cc2)CC1]

LHMDS (1M in THF, 2.61 mL, 2.61 mmol) was added to intermediate 11b (300 mg, 0.75 mmol), intermediate 2b (178 mg, 0.75 mmol), and Pd-PEPPSI-IHept^{C1} (21.74 mg, 0.02 mmol) in THF (2 mL) under nitrogen. The resulting mixture was stirred at 60° C. for 2 h. The reaction mixture was quenched with saturated NH₄Cl (25 mL) and extracted with EtOAc (3×50 mL). The organic layer was dried over Na₂SO₄, filtered and evaporated to afford crude product. The crude product was purified by flash silica chromatography, elution gradient 0 to 60% EtOAc in Et₂O to give the title compound (280 mg, 67.0%) as a yellow solid. ¹H NMR δ 0.87 (6H, t), 1.27-1.4 (6H, m), 1.42-1.52 (5H, m), 1.59-1.76 (3H, m), 1.79-1.98 (3H, m), 2.54 (2H, dd), 2.59-2.7 (2H, m), 2.94-3.04 (1H, m), 3.27 (2H, d), 3.35-3.41 (2H, m), 3.54 (2H, dt), 3.62 (2H, d), 4.16 (1H, d), 6.74-6.93 (4H, m), 7.11-7.18 (2H, m), 8.26 (1H, d), 12.26 (1H, s); m/z: ES⁺ [M+H]⁺=561.0.

### Example 23

### 7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indole-3-carbonitrile

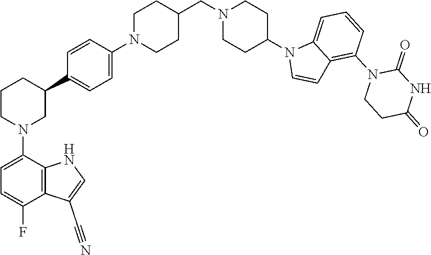 [SMILES: N#Cc1c[nH]c2c(N3CCC[C@@H](c4ccc(N5CCC(CN6CCC(n7ccc8c(N9CCC(=O)NC9=O)cccc87)CC6)CC5)cc4)C3)ccc(F)c12]

Formic acid (2 ml, 52.15 mmol) was added to 7-[(3S)-3-{4-[4-(dibutoxymethyl)piperidin-1-yl]phenyl}piperidin-1-yl]-4-fluoro-1H-indole-3-carbonitrile (150 mg, 0.27 mmol) at RT and the mixture was stirred for 1 h. The solvent was then evaporated and the residue combined with intermediate 5f (84 mg, 0.27 mmol), in NMP (2 mL). The mixture was stirred at RT for 1 h. Purification of the reaction mixture by preparative HPLC (Column A, Eluent A) gave the title compound in the form of a formate salt (27.0 mg, 13.89%) as a white solid. ¹H NMR δ 1.21 (2H, q), 1.47-1.6 (1H, m), 1.65 (1H, s), 1.76-1.88 (3H, m), 1.88-2.06 (6H, m), 2.16 (2H, t), 2.23 (2H, d), 2.54-2.72 (4H, m), 2.76 (2H, t), 2.89-3.07 (3H, m), 3.40 (3H, s), 3.62 (2H, d), 3.77 (2H, t), 4.3-4.46 (1H, m), 6.41 (1H, d), 6.70 (1H, dd), 6.84 (3H, dd), 6.96 (1H, d), 7.14 (3H, t), 7.47-7.6 (2H, m), 8.17 (1H, s), 10.34 (1H, s); m/z: ES⁺ [M+H]+=727.0.

### Example 24

### 7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indole-3-carbonitrile

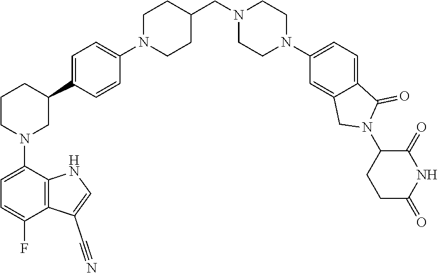 [SMILES: N#Cc1c[nH]c2c(N3CCC[C@@H](c4ccc(N5CCC(CN6CCN(c7ccc8c(c7)CN(C7CCC(=O)NC7=O)C8=O)CC6)CC5)cc4)C3)ccc(F)c12]

Intermediate 23a (140 mg, 0.25 mmol) was stirred in formic acid (1 mL) at RT for 1 h and then evaporated to dryness. Intermediate 1i (107 mg, 0.25 mmol) was also stirred in formic acid (1 mL) at RT for 1 h and then evaporated to dryness. The two residues were then combined in NMP (2 mL) and the resulting mixture was stirred at RT for 1 h. The reaction mixture was purified by C18-flash chromatography, elution gradient 5 to 100% MeCN in water (1% NH₄HCO₃) to give the title compound (53.0 mg, 28.6%) as a white solid. ¹H NMR δ 1.11-1.32 (2H, m), 1.44-1.6 (1H, m), 1.66 (1H, s), 1.74-2 (6H, m), 2.20 (2H, d), 2.3-2.43 (1H, m), 2.48 (3H, d), 2.52-2.72 (6H, m), 2.84-3.04 (2H, m), 3.23-3.33 (6H, m), 3.61 (2H, d), 4.16-4.38 (2H, m), 5.05 (1H, dd), 6.79 (1H, dd), 6.83-6.93 (3H, m), 7.05 (2H, d), 7.15 (2H, d), 7.52 (1H, d), 8.26 (1H, s), 10.96 (1H, s), 12.27 (1H, s); m/z: ES⁺ [M+H]⁺=743.0.

### Example 25

### 4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile

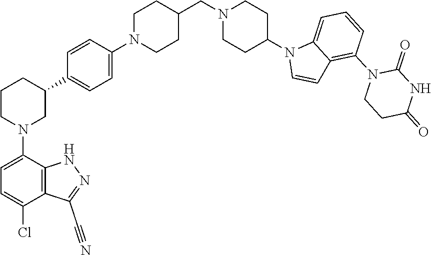 [SMILES: N#Cc1n[nH]c2c(N3CCC[C@H](c4ccc(N5CCC(CN6CCC(n7ccc8c(N9CCC(=O)NC9=O)cccc87)CC6)CC5)cc4)C3)ccc(Cl)c12]

The title compound was prepared according to the procedure described in Example 9 but using (S)-3-(4-bromophenyl)piperidine oxalate in step 9d. Purification by HPLC (Column A, Eluent A) gave the title compound in the form of a formate salt (43.0 mg, 33.4%) as a yellow solid; ¹H NMR δ 0.85 (1H, q), 1.16-1.3 (2H, m), 1.52-1.72 (2H, m), 1.78-1.91 (2H, m), 1.9-2.1 (6H, m), 2.24 (4H, dd), 2.63 (2H, dd), 2.67-2.8 (4H, m), 3.02 (3H, d), 3.51 (3H, q), 3.64 (2H, d), 3.78 (2H, t), 4.39 (1H, tt), 6.42 (1H, d), 6.85-7 (4H, m), 7.11-7.3 (4H, m), 7.53 (2H, dd), 10.33 (1H, s); m/z: ES⁺ [M+H]⁺=744.5.

### Example 26

### 4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile

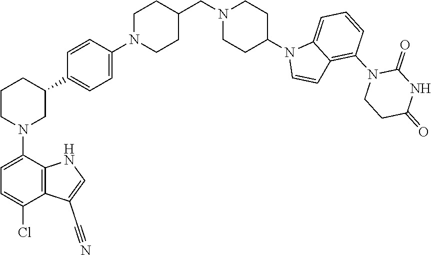 [SMILES: N#Cc1c[nH]c2c(N3CCC[C@H](c4ccc(N5CCC(CN6CCC(n7ccc8c(N9CCC(=O)NC9=O)cccc87)CC6)CC5)cc4)C3)ccc(Cl)c12]

Intermediate 21d was reacted with intermediate 5f using the general synthetic method illustrated by example 23 after purification by HPLC (Column A, Eluent A) to give the title compound in the form of a formate salt (42.0 mg, 54.4%) as a white solid; ¹H NMR δ 0.84 (1H, d), 1.22 (3H, d), 1.57 (1H, t), 1.66 (1H, s), 1.81 (3H, d), 1.94 (5H, d), 2.00 (1H, d), 2.07 (1H, s), 2.17 (2H, t), 2.24 (2H, d), 2.55-2.7 (3H, m), 2.76 (2H, t), 2.99 (3H, d), 3.63 (2H, d), 3.78 (2H, t), 4.37 (1H, s), 6.41 (1H, d), 6.85 (3H, dd), 6.96 (1H, d), 7.1-7.19 (4H, m), 7.47-7.56 (2H, m), 8.31 (1H, s), 10.32 (1H, s), 12.28 (1H, s); m/z: ES⁺ [M+H]⁺=743.0.

### Example 27

### Intermediate 27a

### tert-Butyl 4-[4-(benzyloxy)phenyl]piperidine-1-carboxylate

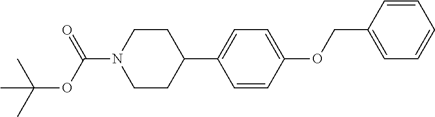 [SMILES: CC(C)(C)OC(=O)N1CCC(c2ccc(OCc3ccccc3)cc2)CC1]

Benzyl bromide (2.89 g, 16.87 mmol) was added to tert-butyl 4-(4-hydroxyphenyl)piperidine-1-carboxylate (3.6 g, 12.98 mmol) and K₂CO₃ (5.38 g, 38.94 mmol) in DMF (52 mL). The resulting mixture was stirred at RT for 5 h. The reaction mixture was diluted with EtOAc (200 mL), washed water (100 mL) and NaCl solution (50 mL). The organic layer was dried over Na₂SO₄, filtered and evaporated to afford crude product. The crude product was purified by flash silica chromatography, elution gradient 0 to 20% EtOAc in Et₂O to give the title compound (4.50 g, 94%) as a white solid. ¹H NMR δ 1.29 (11H, d), 1.59 (2H, d), 2.40-2.55 (1H, m), 2.65 (2H, s), 3.93 (2H, d), 4.94 (2H, d), 6.76-6.86 (2H, m), 6.97-7.08 (2H, m), 7.14-7.37 (5H, m); m/z: ES⁺ [M+H]⁺=353.2.

### Intermediate 27b

### 4-[4-(Benzyloxy)phenyl]piperidine

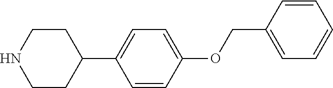 [SMILES: c1ccc(COc2ccc(C3CCNCC3)cc2)cc1]

tert-Butyl 4-[4-(benzyloxy)phenyl]piperidine-1-carboxylate (4.5 g, 12.25 mmol) was stirred in formic acid (50 mL) at RT for 2 h. The reaction mixture was then evaporated to dryness to afford crude product. The crude product was purified by ion exchange chromatography, using an SCX column. The desired product was eluted from the column using 7M NH₃/MeOH to give the title compound (3.20 g, 98%) as a white solid. ¹H NMR δ 1.33-1.52 (2H, m), 1.56-1.68 (2H, m), 2.39-2.60 (2H, m), 2.63 (2H, s), 2.92-3.03 (2H, m), 5.04 (2H, s), 6.85-6.96 (2H, m), 7.05-7.16 (2H, m), 7.24-7.47 (5H, m); m/z: ES⁺ [M+H]⁺=268.2.

### Intermediate 27c

### 7-{4-[4-(Benzyloxy)phenyl]piperidin-1-yl}-4-methyl-1H-indole-3-carbonitrile

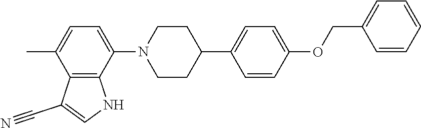 [SMILES: Cc1ccc(N2CCC(c3ccc(OCc4ccccc4)cc3)CC2)c2[nH]cc(C#N)c12]

Pd-PEPPSI-IPent (0.304 g, 0.31 mmol) was added to 4-[4-(benzyloxy)phenyl]piperidine (3.20 g, 11.99 mmol), intermediate 8a (2.45 g, 10.42 mmol) and LHMDS (1M in THF, 36.5 ml, 36.48 mmol) in 1,4-dioxane (25 mL) under nitrogen. The resulting mixture was stirred at 60° C. for 2 h. The reaction mixture was cooled to RT and was quenched with water (5 mL). The solvent was removed under reduced pressure to afford crude product. The crude product was purified by flash silica chromatography, elution gradient 0 to 100% DCM in Et₂O followed by 0 to 10% EtOAc in DCM to give the title compound (2.60 g, 59.2%) as a yellow solid. ¹H NMR δ 1.85 (2H, d), 1.89-2.08 (2H, m), 2.60 (4H, s), 2.67-2.82 (2H, m), 3.38 (2H, d), 5.09 (2H, s), 6.77 (1H, d), 6.88 (1H, d), 6.93-7.04 (2H, m), 7.17-7.28 (2H, m), 7.30-7.51 (5H, m), 8.17 (1H, d), 11.88-11.95 (1H, m); m/z: ES⁺ [M+H]⁺=422.3.

### Intermediate 27d

### 7-{4-[4-(Benzyloxy)phenyl]piperidin-1-yl}-4-methyl-1-{[2-(trimethylsilyl)ethoxy]methyl}-1H-indole-3-carbonitrile

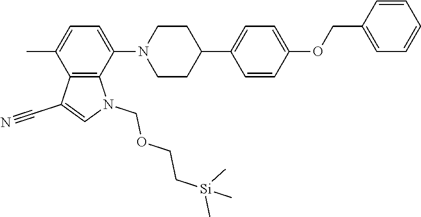 [SMILES: Cc1ccc(N2CCC(c3ccc(OCc4ccccc4)cc3)CC2)c2c1c(C#N)cn2COCC[Si](C)(C)C]

Sodium hydride (0.144 g, 3.61 mmol) was added to 7-{4-[4-(benzyloxy)phenyl]piperidin-1-yl}-4-methyl-1H-indole-3-carbonitrile (1.17 g, 2.78 mmol) in THF (18 mL) at 0° C. and stirred for 1 h. 2-(Trimethylsilyl)ethoxymethyl chloride (0.591 ml, 3.33 mmol) was added and the resulting solution was stirred at RT for 2 h. The reaction mixture was quenched with water and the reaction was evaporated to afford crude product. The crude product was purified by flash silica chromatography, elution gradient 0 to 90% DCM in Et₂O to give the title compound (1.3 g, 85%) as a white solid. ¹H NMR δ −0.09 (9H, s), 0.76-0.87 (2H, m), 1.85 (4H, s), 2.57 (4H, d), 2.84 (2H, s), 3.16 (2H, d), 3.48-3.59 (2H, m), 5.09 (2H, s), 5.97 (2H, s), 6.92-7.04 (3H, m), 7.12 (1H, d), 7.18-7.26 (2H, m), 7.28-7.51 (5H, m), 8.36 (1H, s); m/z: ES⁺ [M+H]⁺=552.4.

### Intermediate 27e

### 7-[4-(4-Hydroxyphenyl)piperidin-1-yl]-4-methyl-1-{[2-(trimethylsilyl)ethoxy]methyl}-1H-indole-3-carbonitrile

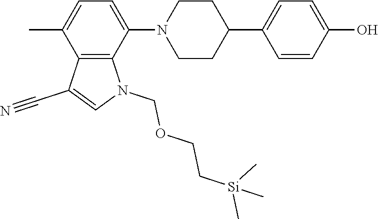 [SMILES: Cc1ccc(N2CCC(c3ccc(O)cc3)CC2)c2c1c(C#N)cn2COCC[Si](C)(C)C]

Pd/C (10%, 1.24 g, 11.65 mmol) was added to 7-{4-[4-(benzyloxy)phenyl]piperidin-1-yl}-4-methyl-1-{[2-(trimethylsilyl)ethoxy]methyl}-1H-indole-3-carbonitrile (1.24 g, 2.25 mmol) in EtOAc (60 mL) under hydrogen and stirred at RT for 2 h. The reaction mixture was filtered through Celite® and the solvent was evaporated to give the title compound (0.94 g, 91%) as a white solid. ¹H NMR (CDCl₃) δ −0.03 (9H, s), 0.83-1.01 (2H, m), 1.83-2.01 (4H, m), 2.56-2.69 (1H, m), 2.77 (3H, s), 2.93 (2H, td), 3.29 (2H, d), 3.50-3.61 (2H, m), 6.06 (2H, s), 6.79-6.90 (2H, m), 6.96-7.04 (1H, m), 7.12 (1H, d), 7.15-7.23 (2H, m), 7.73 (1H, s) OH not observed; m/z: ES⁺ [M+H]⁺=462.3.

### Intermediate 27f

### 7-(4-{4-[3-(1,3-Dioxolan-2-yl)propoxy]phenyl}piperidin-1-yl)-4-methyl-1-{[2-(trimethylsilyl)ethoxy]methyl}-1H-indole-3-carbonitrile

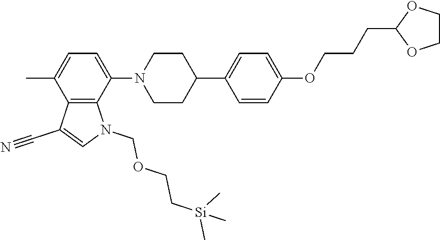 [SMILES: Cc1ccc(N2CCC(c3ccc(OCCCC4OCCO4)cc3)CC2)c2c1c(C#N)cn2COCC[Si](C)(C)C]

K₂CO₃ (0.808 g, 5.85 mmol) was added to 7-[4-(4-hydroxyphenyl)piperidin-1-yl]-4-methyl-1-{[2-(trimethylsilyl)ethoxy]methyl}-1H-indole-3-carbonitrile (0.90 g, 1.95 mmol) and 2-(3-bromopropyl)-1,3-dioxolane (0.760 g, 3.90 mmol) in DMF (10 mL). The resulting mixture was stirred at 80° C. for 8 h. The reaction mixture was cooled to RT and purified by flash C18-flash chromatography, elution gradient 0 to 100% MeOH in water (0.1% NH₄HCO₃) to give the title compound (0.75 g, 66.8%) as a yellow solid. ¹H NMR (MeOD) δ 0.70 (9H, s), 1.60 (2H, t), 2.02 (1H, s), 2.46-2.60 (3H, m), 2.63 (5H, s), 3.41 (3H, s), 3.90-4.00 (2H, m), 4.11 (2H, s), 4.32 (2H, t), 4.48-4.81 (6H, m), 5.64 (1H, t), 6.76 (2H, s), 7.66 (2H, d), 7.78 (1H, d), 7.91 (1H, d), 7.99 (2H, d), 9.15 (1H, s); m/z: ES⁺ [M+H]⁺=576.5.

### Intermediate 27g

### 7-(4-{4-[3-(1,3-Dioxolan-2-yl)propoxy]phenyl}piperidin-1-yl)-4-methyl-1H-indole-3-carbonitrile

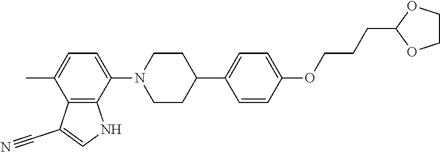 [SMILES: Cc1ccc(N2CCC(c3ccc(OCCCC4OCCO4)cc3)CC2)c2[nH]cc(C#N)c12]

Tetrabutylammonium fluoride (1M in THF, 9.3 mL, 9.38 mmol) was added to 7-(4-{4-[3-(1,3-dioxolan-2-yl)propoxy]phenyl}piperidin-1-yl)-4-methyl-1-{[2-(trimethylsilyl)ethoxy]methyl}-1H-indole-3-carbonitrile (360 mg, 0.63 mmol) and the resulting mixture was stirred at 40° C. for 16 h. The reaction was cooled to RT and the mixture was evaporated to dryness to afford crude product. The crude product was purified by flash silica chromatography, elution gradient 0 to 100% DCM in Et₂O followed by 0 to 12% EtOAc in DCM to give the title compound (0.19 g, 68.2%) as a white solid. ¹H NMR δ 1.73 (3H, m), 1.81-1.89 (3H, m), 1.98 (2H, qd), 2.61 (4H, d), 2.70-2.80 (2H, m), 3.38 (2H, d), 3.73-3.82 (2H, m), 3.87-3.92 (2H, m), 3.97 (2H, t), 4.86 (1H, t), 6.77 (1H, d), 6.89 (3H, dd), 7.20 (2H, d), 8.17 (1H, d), 11.91 (1H, d); m/z: ES⁺ [M+H]⁺=446.35.

### Example 27

### 7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indole-3-carbonitrile

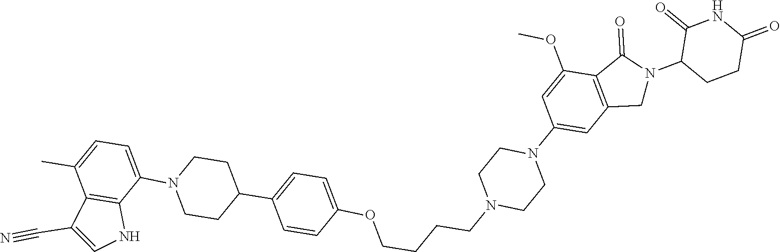 [SMILES: COc1cc(N2CCN(CCCCOc3ccc(C4CCN(c5ccc(C)c6c(C#N)c[nH]c56)CC4)cc3)CC2)cc2c1C(=O)N(C1CCC(=O)NC1=O)C2]

7-(4-{4-[3-(1,3-Dioxolan-2-yl)propoxy]phenyl}piperidin-1-yl)-4-methyl-1H-indole-3-carbonitrile (0.159 g, 0.31 mmol) and intermediate 7c (0.162 g, 0.35 mmol) were heated in formic acid (4 mL) to 40° C. for 1 h. The reaction was cooled to RT and evaporated to dryness. NMP (4 mL) was added to the residue and the resulting solution was stirred at RT for 16 h. The reaction mixture was quenched with NaHCO₃ solution (10 mL), extracted with DCM (30 mL) and the organic layer was dried over MgSO₄, filtered and evaporated to afford crude product. The crude product was purified by flash silica chromatography, elution gradient 0 to 10% MeOH in DCM to give the title compound (0.11 g, 48.2%) as a white solid. ¹H NMR δ 1.50-1.65 (2H, m), 1.67-1.84 (2H, m), 1.87-2.00 (5H, m), 2.18-2.46 (6H, m), 2.57 (5H, s), 2.59-2.78 (3H, m), 2.80-2.90 (1H, m), 3.26 (4H, d), 3.33-3.35 (2H, m), 3.81 (3H, s), 3.97 (2H, t), 4.02-4.27 (2H, m), 4.94 (1H, dd), 6.47 (1H, d), 6.58 (1H, s), 6.75 (1H, d), 6.82-6.93 (3H, m), 7.18 (2H, d), 8.15 (1H, d), 10.88 (1H, s), 11.89 (1H, s); m/z: ES⁺ [M+H]⁺=744.4.

### Example 28

### 7-{4-[4-(4-{4-[2-(2,6-dioxopiperidin-3-yl)-1-oxo-2,3-dihydro-1H-isoindol-5-yl]piperazin-1-yl}butoxy)phenyl]piperidin-1-yl}-4-methyl-1H-indole-3-carbonitrile

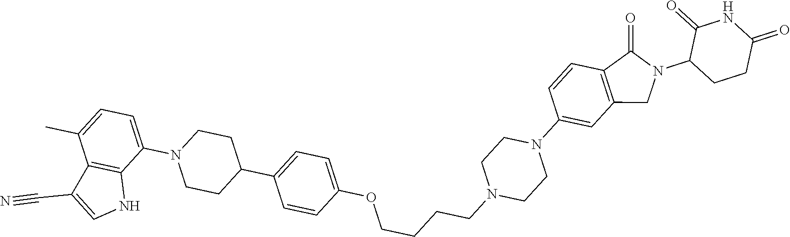 [SMILES: Cc1ccc(N2CCC(c3ccc(OCCCCN4CCN(c5ccc6c(c5)CN(C5CCC(=O)NC5=O)C6=O)CC4)cc3)CC2)c2[nH]cc(C#N)c12]

Intermediate 27g was reacted with intermediate 1i using the general synthetic method illustrated by example 27 to give the title compound (31.6 mg, 12.75%) as a white solid. H NMR δ 1.51 (2H, q), 1.58-1.76 (4H, m), 1.77-1.90 (3H, m), 2.25 (6H, q), 2.47 (5H, s), 2.62 (3H, t), 2.75 (1H, d), 3.15 (4H, t), 3.26 (2H, d), 3.87 (2H, t), 4.03-4.26 (2H, m), 4.92 (1H, dd), 6.65 (1H, d), 6.71-6.83 (3H, m), 6.93 (2H, d), 7.08 (2H, d), 7.40 (1H, d), 8.04 (1H, d), 10.81 (1H, s), 11.77 (1H, s); m/z: ES⁺ [M+H]⁺=714.3.

### Example 29

### 7-{4-[4-(4-{4-[4-(2,4-Dioxo-1,3-diazinan-1-yl)-1H-indol-1-yl]piperidin-1-yl}butoxy)phenyl]piperidin-1-yl}-4-methyl-1H-indole-3-carbonitrile

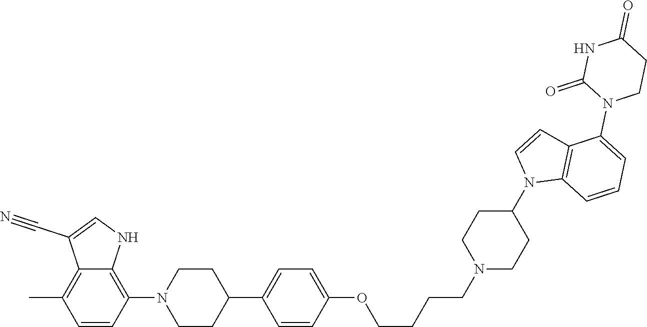 [SMILES: Cc1ccc(N2CCC(c3ccc(OCCCCN4CCC(n5ccc6c(N7CCC(=O)NC7=O)cccc65)CC4)cc3)CC2)c2[nH]cc(C#N)c12]

The title compound was prepared using methodology described in example 23 using intermediate 27g and intermediate 5f to give the title compound in the form of a formate salt (14.4 mg, 53.0%) as a beige solid. ¹H NMR δ 1.77-1.91 (6H, m), 1.93-2.05 (3H, m), 2.16-2.29 (4H, m), 2.55-2.66 (4H, m), 2.72-2.83 (4H, m), 3.09-3.29 (3H, m), 3.40 (2H, d), 3.64-3.75 (1H, m), 3.79 (2H, t), 3.99-4.11 (2H, m), 4.62-4.87 (1H, m), 6.41-6.55 (1H, m), 6.79 (1H, d), 6.87-6.91 (1H, m), 6.94 (2H, d), 7.01 (1H, d), 7.1-7.3 (3H, m), 7.37-7.49 (1H, m), 7.56 (1H, d), 8.17 (1H, d), 9.09-9.33 (1H, m), 10.34 (1H, s), 11.84-11.98 (1H, m); m/z: ES⁺ [M+H]⁺=698.4.

### Example 30

### Intermediate 30a

### 6-Chloro-2-methoxy-4-methylpyridine-3-carboxylic acid

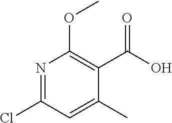

To a stirred solution sodium methoxide (20.67 ml, 112 mmol) in THF (50.0 mL) was added 2,6-dichloro-4-methylpyridine-3-carboxylic acid (10.0 g, 48.5 mmol) in THF (50.0 ml) at 0° C. The reaction mixture was stirred at 70° C. for 16 h. The reaction was then cooled to RT and acidified with a HCl (2M, aq) to pH 3-4, then extracted with EtOAc (160 mL). The organic layer was washed with water (100 mL), NaCl solution (100 mL), dried over Na₂SO₄, filtered and evaporated to dryness to give the title compound (8.0 g, 78% yield) which was used without further purification. ¹H NMR δ 2.27 (3H, s), 3.87 (3H, s), 7.10 (1H, s), 13.42 (1H, s). no mass ion.

### Intermediate 30b

### Methyl 6-chloro-2-methoxy-4-methylpyridine-3-carboxylate

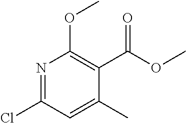

To a stirred solution of 6-chloro-2-methoxy-4-methylpyridine-3-carboxylic acid (8.0 g, 39.7 mmol) in DMF (80 mL) was added K₂CO₃ (6.58 g, 47.6 mmol) and methyl iodide (8.45 g, 59.5 mmol) which was stirred at RT for 16 h. The reaction mixture was diluted with water (80 mL) and extracted with MTBE (240 mL). The organic layer was washed with NaCl solution (160 mL, dried over Na₂SO₄, filtered and evaporated to dryness to give the title compound (8.5 g, 37.1 mmol, 93% yield) as pale yellow solid. ¹H NMR δ 2.26 (3H, s), 3.84 (3H, s), 3.87 (3H, s), 7.13 (1H, s).

### Intermediate 30c

### Methyl 4-(bromomethyl)-6-chloro-2-methoxypyridine-3-carboxylate

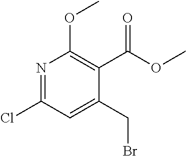

Methyl 6-chloro-2-methoxy-4-methylpyridine-3-carboxylate (8.0 g, 37.1 mmol) was added to tert-butyl acetate (150 mL) under nitrogen. NBS (9.24 g, 51.9 mmol) and benzoyl peroxide (1.79 g, 7.42 mmol) were added at RT and was then stirred at 110° C. for 14 h. Additional NBS (2.64 g, 14.84 mmol) was added and was continued to stir at 110° C. for 12 h. The reaction mixture was cooled to RT and diluted with NaHCO₃ solution (10%, 50 mL) and extracted with EtOAc (120 mL). The organic layer was washed with NaCl solution (80 mL), dried over Na₂SO₄, filtered and evaporated to dryness to give the crude title compound (11.0 g, 10.01 mmol, 27.0% yield) as brown liquid which was used without further purification. m/z: ES⁺ [M+H]⁺=294.0. no nmr data

### Intermediate 30d

### 3-(6-Chloro-4-methoxy-3-oxo-1,3-dihydro-2H-pyrrolo[3,4-c]pyridin-2-yl)piperidine-2,6-dione

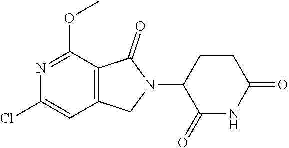 [SMILES: COc1nc(Cl)cc2c1C(=O)N(C1CCC(=O)NC1=O)C2]

Methyl 4-(bromomethyl)-6-chloro-2-methoxypyridine-3-carboxylate (11.0 g, 10.01 mmol) was dissolved in MeCN (80 mL). 3-Aminopiperidine-2,6-dione hydrochloride (1.65 g, 10.01 mmol) and DIPEA (5.17 g, 40.0 mmol) were added and the reaction mixture was stirred at 85° C. for 16 h. The reaction mixture was then cooled to RT and was diluted with DCM (200 mL), washed AcOH (5%, aq 200 mL), water (200 mL), NaHCO₃ solution (200 mL), NaCl solution (200 mL), dried with Na₂SO₄, filtered and evaporated to afford crude product. The crude product was triturated with EtOAc (10 mL) and washed with MTBE (30 mL) to afford solid which was collected by filtration and dried under vacuum to give the title compound (1.6 g, 4.35 mmol, 43.5% yield) as brown solid. ¹H NMR δ 1.89-2.02 (1H, m), 2.30-2.35 (1H, m), 2.50-2.59 (1H, m), 2.84-2.98 (1H, m), 3.99 (3H, s), 4.26-4.39 (1H, m), 4.40-4.53 (1H, m), 5.05 (1H, dd), 7.40 (1H, s), 10.99 (1H, br s); m/z: ES⁺ [M+H]⁺=310.2.

### Intermediate 30e

### tert-Butyl 4-[2-(2,6-dioxopiperidin-3-yl)-4-methoxy-3-oxo-2,3-dihydro-1H-pyrrolo[3,4-c]pyridin-6-ylpiperazine-1-carboxylate

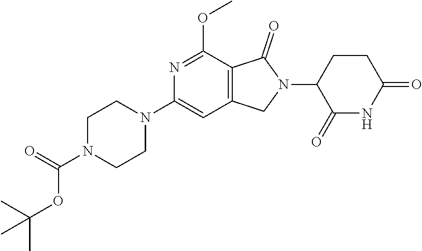 [SMILES: COc1nc(N2CCN(C(=O)OC(C)(C)C)CC2)cc2c1C(=O)N(C1CCC(=O)NC1=O)C2]

Pd-PEPPSI-IPent (47.1 mg, 0.048 mmol) was added to tert-butyl piperazine-1-carboxylate (541 mg, 2.91 mmol), Cs₂CO₃ (947 mg, 2.91 mmol) and 3-(6-chloro-4-methoxy-3-oxo-1,3-dihydro-2H-pyrrolo[3,4-c]pyridin-2-yl)piperidine-2,6-dione (300 mg, 0.969 mmol) in 1,4-dioxane (10 mL) at RT under nitrogen. The resulting suspension was stirred at 120° C. for 2 h. The reaction mixture was cooled to RT and diluted with DCM (20 mL), washed with AcOH (5%, aq 20 mL), water (20 mL), NaHCO₃ solution (20 mL), NaCl solution (20 mL), dried with Na₂SO₄, filtered and evaporated to afford crude product. The crude product was triturated with EtOAc (4 mL) and washed with MTBE (16 mL) to afford solid which was collected by filtration and dried under vacuum to give the title compound (260 mg, 0.485 mmol, 50.1% yield) as brown solid. ¹H NMR δ 1.43 (9H, s), 1.88-2.00 (1H, m), 2.29-2.39 (1H, m), 2.58-2.68 (1H, m), 2.86-2.96 (1H, m), 3.34-3.44 (4H, m), 3.53-3.63 (4H, m), 3.90 (3H, s), 4.09-4.18 (1H, m), 4.24-4.34 (1H, m), 4.97 (1H, br dd), 6.50 (1H, s), 10.92 (1H, br s); m/z: ES⁺ [M+H]⁺=460.2.

### Example 30

### 7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-methyl-1H-indole-3-carbonitrile

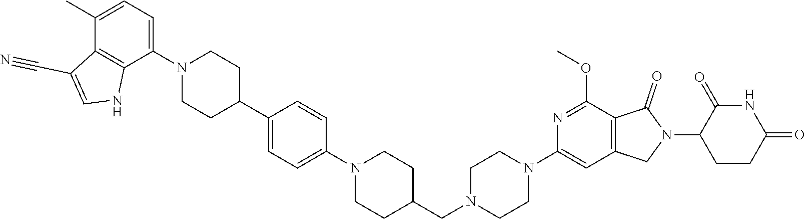 [SMILES: COc1nc(N2CCN(CC3CCN(c4ccc(C5CCN(c6ccc(C)c7c(C#N)c[nH]c67)CC5)cc4)CC3)CC2)cc2c1C(=O)N(C1CCC(=O)NC1=O)C2]

Intermediate 8b (59 mg, 0.13 mmol) and tert-butyl 4-[2-(2,6-dioxopiperidin-3-yl)-4-methoxy-3-oxo-2,3-dihydro-1H-pyrrolo[3,4-c]pyridin-6-yl]piperazine-1-carboxylate (63.4 mg, 0.14 mmol) were heated in formic acid (3 mL, 79.52 mmol) at 60° C. for 2 h. The reaction mixture was concentrated and the crude residue was suspended in NMP (2 mL) at RT. The reaction mixture was stirred for 5 mins and sodium triacetoxyborohydride (66.4 mg, 0.31 mmol) was added. The resulting suspension was stirred at RT for 16 h. NaHCO₃ solution was added and the solids formed were collected by filtration, washed with water (5 mL) and dried over vacuum to afford crude product. Purification by preparative HPLC (Column A, Eluent A) gave the title compound in the form of a formate salt (3.0 mg, 3%) as a yellow solid. ¹H NMR δ 1.17-1.39 (9H, m), 1.75-2.05 (11H, m), 2.23 (3H, d), 2.60 (3H, s), 2.75 (1H, t), 3.35-3.41 (2H, m), 3.58-3.7 (6H, m), 3.90 (3H, s), 4.08-4.2 (1H, m), 4.22-4.34 (1H, m), 4.9-5.02 (1H, m), 6.49 (1H, s), 6.78 (1H, d), 6.86-6.95 (3H, m), 7.14 (2H, d), 8.11-8.23 (1H, m), 10.91 (1H, s), 11.90 (1H, s); m/z: ES⁺ [M+H]⁺=770.5.

### Example 31

### 7-{4-[4-(4-{4-[2-(2,6-Dioxopiperidin-3-yl)-4-methoxy-3-oxo-2,3-dihydro-1H-pyrrolo[3,4-c]pyridin-6-yl]piperazin-1-yl}butoxy)phenyl]piperidin-1-yl}-4-methyl-1H-indole-3-carbonitrile

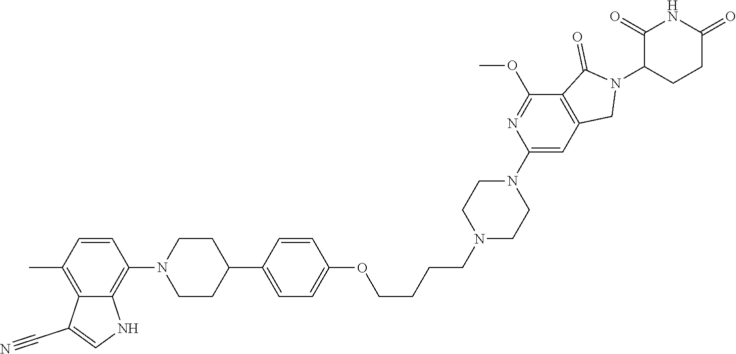 [SMILES: COc1nc(N2CCN(CCCCOc3ccc(C4CCN(c5ccc(C)c6c(C#N)c[nH]c56)CC4)cc3)CC2)cc2c1C(=O)N(C1CCC(=O)NC1=O)C2]

Intermediate 27g (28 mg, 0.07 mmol) and intermediate 30e (32 mg, 0.07 mmol) were stirred in formic acid (6 mL) at 60° C. for 1 h. The mixture was evaporated and diluted with NMP (1 mL). Sodium triacetoxyborohydride (37 mg, 0.17 mmol) was added and the mixture was stirred at RT for 30 mins. The reaction was neutralised with NaHCO₃ solution until pH 12. The solid was filtered under vacuum and washed with water (5 mL). The solid was added to a mixture of EtOAc:Heptane (1:1, 70 mL) with addition of DCM (5 mL) and stirred at 50° C. for 16 h. The suspension was filtered hot and the solids were washed with cold EtOAc:Heptane (1:1) and dried under vacuum to give the title compound (20.0 mg, 36.3%) as a pale green solid. ¹H NMR (CDCl₃) δ 1.77-1.89 (4H, m), 1.91-2.04 (4H, m), 2.1-2.2 (1H, m), 2.26 (1H, qd), 2.63-2.71 (3H, m), 2.74 (3H, s), 2.75-2.96 (8H, m), 3.42 (2H, d), 3.69-3.85 (4H, m), 3.95-4.04 (5H, m), 4.18 (1H, d), 4.35 (1H, d), 5.13 (1H, dd), 6.17 (1H, s), 6.77-6.9 (2H, m), 6.91 (1H, d), 6.94-6.98 (1H, m), 7.19 (2H, d), 7.73 (1H, d), 7.96 (1H, s), 8.19 (1H, s), 9.20 (1H, s); m/z: ES⁺ [M+H]⁺=745.4.

### Example 32

### Intermediate 32a

### Benzyl 4-(4-hydroxyphenyl)piperidine-1-carboxylate

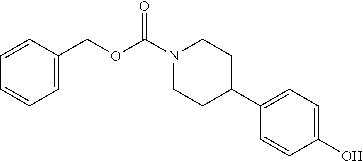 [SMILES: O=C(OCc1ccccc1)N1CCC(c2ccc(O)cc2)CC1]

Benzyl carbonochloridate (7.3 ml, 51.47 mmol) was added to triethylamine (19.5 ml, 140.38 mmol) and 4-(piperidin-4-yl)phenol HCl (10 g, 46.79 mmol) in THF (441.0 mL). The resulting mixture was stirred at RT for 16 h. The reaction mixture was diluted with EtOAc (100 mL) and washed with NaHCO₃ solution (2×100 mL). The organic layer was dried over Na2SO4, filtered and evaporated to afford crude product. The crude product was purified by flash silica chromatography, elution gradient 0 to 40% EtOAc in Et₂O to give the title compound (1.5 g, 10.29%) as a white solid. ¹H NMR δ 1.32-1.55 (2H, m), 1.70 (2H, d), 2.49-2.63 (1H, m), 2.85 (2H, s), 4.10 (2H, d), 5.07 (2H, s), 6.60-6.71 (2H, m), 6.94-7.05 (2H, m), 7.36 (5H, d), 9.14 (1H, s); m/z: ES⁺ [M+H]⁺=312.2.

### Intermediate 32b

### Benzyl 4-{4-[3-(1,3-dioxolan-2-yl)propoxy]phenyl}piperidine-1-carboxylate

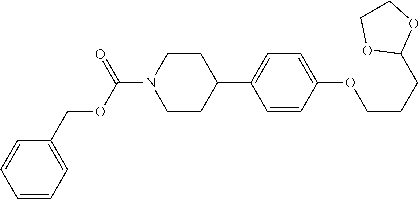 [SMILES: O=C(OCc1ccccc1)N1CCC(c2ccc(OCCCC3OCCO3)cc2)CC1]

K₂CO₃ (1.59 g, 11.56 mmol) was added to benzyl 4-(4-hydroxyphenyl)piperidine-1-carboxylate (1.8 g, 5.78 mmol) and 2-(3-bromopropyl)-1,3-dioxolane (2.25 g, 11.56 mmol) in MeCN (15 mL). The resulting mixture was stirred at 80° C. for 16 h. The reaction was cooled to RT, filtered under vacuum and evaporated to afford crude product. The crude product was purified by flash silica chromatography, elution gradient 0 to 50% EtOAc in Et₂O to give the title compound (1.7 g, 69.1%) as a white solid. ¹H NMR (CDCl₃) δ 1.58-1.71 (3H, m), 1.82 (2H, s), 1.81-1.99 (3H, m), 2.57-2.69 (1H, m), 2.89 (2H, s), 3.78-3.99 (2H, m), 3.96-4.06 (3H, m), 4.33 (3H, s), 4.95 (1H, t), 5.18 (2H, s), 6.77-6.90 (2H, m), 7.03-7.16 (2H, m), 7.29-7.38 (1H, m), 7.34-7.44 (4H, m); m/z: ES⁺ [M+H]⁺=426.2.

### Intermediate 32c

### 4-{4-[3-(1,3-Dioxolan-2-yl)propoxy]phenyl}piperidine

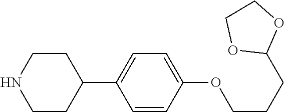 [SMILES: c1cc(C2CCNCC2)ccc1OCCCC1OCCO1]

Pd/C (10%, 0.85 g, 0.80 mmol) was added to benzyl 4-{4-[3-(1,3-dioxolan-2-yl)propoxy]phenyl}piperidine-1-carboxylate (1.7 g, 4.00 mmol) in EtOAc (15.0 mL) under an atmosphere of hydrogen. The resulting mixture was stirred at RT for 4 h. The mixture was filtered through Celite® and evaporated to dryness to give the title compound (1.1 g, 94%) as a white solid, which was used without further purification. ¹H NMR (CDCl₃) δ 1.65 (3H, m), 1.88 (5H, s), 2.33 (2H, s), 2.52-2.66 (1H, m), 2.77 (2H, td), 3.23 (2H, d), 3.79-3.97 (2H, m), 3.95-4.02 (3H, m), 4.96 (1H, t), 6.82-6.91 (2H, m), 7.10-7.24 (2H, m); m/z: ES⁺ [M+H]⁺=292.1.

### Intermediate 32d

### 4-Chloro-7-(4-{4-[3-(1,3-dioxolan-2-yl)propoxy]phenyl}piperidin-1-yl)-1H-indole-3-carbonitrile

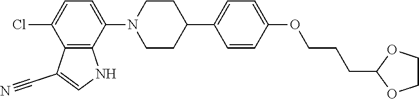 [SMILES: N#Cc1c[nH]c2c(N3CCC(c4ccc(OCCCC5OCCO5)cc4)CC3)ccc(Cl)c12]

4-{4-[3-(1,3-Dioxolan-2-yl)propoxy]phenyl}piperidine was reacted with intermediate 1b using the general synthetic method illustrated by intermediate 11c to give the title compound (266 mg, 55%) as a white solid.

¹H NMR (CDCl₃) δ 1.79-1.97 (3H, m), 1.98 (6H, s), 2.65 (1H, d), 2.90 (2H, s), 3.40 (2H, d), 3.79-3.96 (2H, m), 3.98-4.01 (3H, m), 4.95 (1H, t), 6.81-6.95 (3H, m), 7.13-7.23 (3H, m), 7.76 (1H, s), 9.02 (1H, s); m/z: ES⁺ [M+H]⁺=466.0.

### Example 32

### 4-Chloro-7-{4-[4-(4-{4-[2-(2,6-dioxopiperidin-3-yl)-4-methoxy-3-oxo-2,3-dihydro-1H-pyrrolo[3,4-c]pyridin-6-yl]piperazin-1-yl}butoxy)phenyl]piperidin-1-yl}-1H-indole-3-carbonitrile

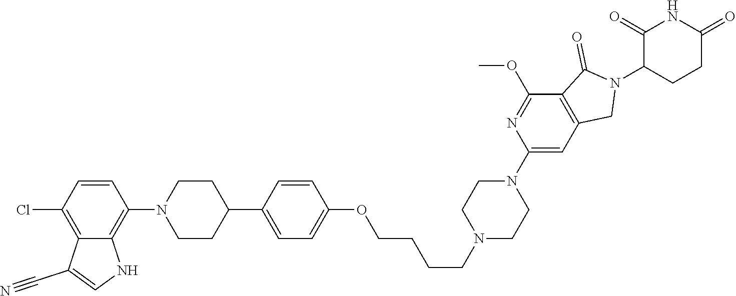 [SMILES: COc1nc(N2CCN(CCCCOc3ccc(C4CCN(c5ccc(Cl)c6c(C#N)c[nH]c56)CC4)cc3)CC2)cc2c1C(=O)N(C1CCC(=O)NC1=O)C2]

4-Chloro-7-(4-{4-[3-(1,3-dioxolan-2-yl)propoxy]phenyl}piperidin-1-yl)-1H-indole-3-carbonitrile was reacted with intermediate 30e using the general synthetic method illustrated by example 23 to give the title compound in the form of a formate salt (20.0 mg, 36.3%) as a white solid. ¹H NMR δ 1.56-1.76 (2H, m), 1.76-1.87 (2H, m), 1.87-2.11 (5H, m), 2.31-2.49 (4H, m), 2.84 (2H, t), 2.9-3.05 (1H, m), 3.48 (3H, d), 3.6-3.76 (4H, m), 3.95 (3H, s), 4.05 (2H, t), 4.19 (1H, d), 4.33 (1H, d), 5.02 (1H, dd), 6.54 (1H, s), 6.83-7.07 (3H, m), 7.15-7.38 (3H, m), 8.25 (2H, s), 8.38 (1H, s), 10.96 (1H, s) 3H under DMSO; m/z: ES⁺ [M+H]⁺=766.6.

### Example 33

### Intermediate 33a

### 7-(4-{4-[3-(1,3-Dioxolan-2-yl)propoxy]phenyl}piperidin-1-yl)-4-fluoro-1H-indole-3-carbonitrile

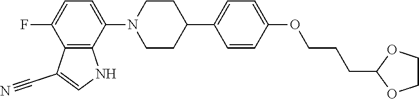 [SMILES: N#Cc1c[nH]c2c(N3CCC(c4ccc(OCCCC5OCCO5)cc4)CC3)ccc(F)c12]

Intermediate 32c was reacted with intermediate 2b using the general synthetic method illustrated by intermediate 11c to give the title compound (240 mg, 52%) as a yellow solid. ¹H NMR δ 1.77 (4H, s), 1.65-2.04 (4H, m), 2.60 (1H, s), 2.67-2.81 (2H, m), 3.36 (2H, d), 3.75 (1H, s), 3.73-3.84 (1H, m), 3.84-4.03 (4H, m), 4.83 (1H, t), 6.80 (1H, dd), 6.86 (1H, d), 6.85-6.97 (2H, m), 7.14-7.23 (2H, m), 8.25 (1H, d), 12.24 (1H, s); m/z: ES⁺ [M+H]⁺=450.2.

### Example 33

### 7-{4-[4-(4-{4-[4-(2,4-Dioxo-1,3-diazinan-1-yl)-1H-indol-1-yl]piperidin-1-yl}butoxy)phenyl]piperidin-1-yl}-4-fluoro-1H-indole-3-carbonitrile

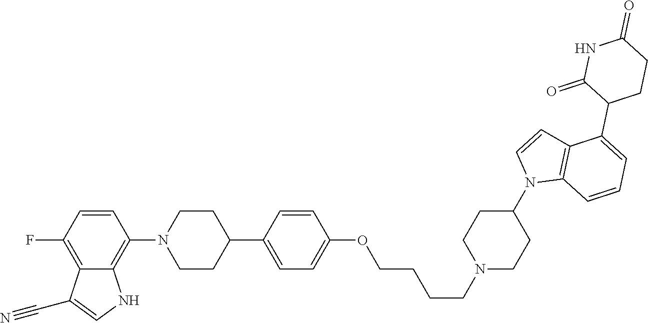 [SMILES: N#Cc1c[nH]c2c(N3CCC(c4ccc(OCCCCN5CCC(n6ccc7c(C8CCC(=O)NC8=O)cccc76)CC5)cc4)CC3)ccc(F)c12]

7-(4-{4-[3-(1,3-Dioxolan-2-yl)propoxy]phenyl}piperidin-1-yl)-4-fluoro-1H-indole-3-carbonitrile was reacted with intermediate 5f using the general synthetic method illustrated by example 23 and purification by HPLC (Column A, Eluent A) gave the title compound in the form of a formate salt (24 mg, 36%) as a white solid. ¹H NMR δ 1.64-1.76 (2H, m), 1.78-1.96 (4H, m), 1.96-2.1 (6H, m), 2.21-2.34 (2H, m), 2.49-2.53 (2H, m), 2.64-2.7 (1H, m), 2.78-2.88 (4H, m), 3.12 (2H, d), 3.42-3.5 (2H, m), 3.83 (2H, t), 4.06 (2H, t), 4.38-4.54 (1H, m), 6.43-6.54 (1H, m), 6.84-6.92 (1H, m), 6.95-7.01 (3H, m), 7.01-7.04 (1H, m), 7.16-7.25 (1H, m), 7.25-7.31 (2H, m), 7.52-7.63 (2H, m), 8.20 (1H, s), 8.32 (1H, s), 10.37 (1H, s); m/z: ES⁺ [M+H]⁺=702.9.

### Example 34

### 4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indole-3-carbonitrile

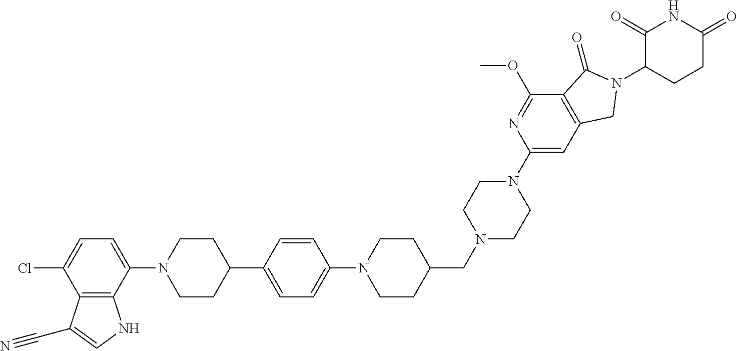 [SMILES: COc1nc(N2CCN(CC3CCN(c4ccc(C5CCN(c6ccc(Cl)c7c(C#N)c[nH]c67)CC5)cc4)CC3)CC2)cc2c1C(=O)N(C1CCC(=O)NC1=O)C2]

Intermediate 1f was reacted with intermediate 30e using the general synthetic method illustrated by example 7 to give the title compound (59.0 mg, 43%) as a yellow solid. ¹H NMR δ 1.19-1.31 (3H, m), 1.70 (1H, s), 1.84 (4H, s), 1.9-2 (3H, m), 2.23 (2H, d), 2.26-2.32 (1H, m), 2.47 (4H, d), 2.54-2.67 (3H, m), 2.79 (2H, t), 2.84-2.95 (1H, m), 3.38-3.47 (2H, m), 3.65 (6H, d), 3.90 (3H, s), 4.14 (1H, d), 4.28 (1H, d), 4.96 (1H, dd), 6.49 (1H, s), 6.89 (3H, dd), 7.16 (3H, dd), 8.32 (1H, s), 10.91 (1H, s), 12.26 (1H, s); m/z: ES⁺ [M+H]⁺=790.8.

### Examples 35 and 36

The enantiomers of Example 34 were separated by SFC purification on a Sepiatec 100 SFC using SFC conditions Regis (R,R) Whelk-01, 21.1×250 mm, 5 micron, 50% MeOH/DCM/50% scCO2 at a flow rate of 60 m/min, 40° C. to give, in order of elution, example 35 (isomer1, 61 mg, 29%) and example 36 (isomer 2, 68 mg, 32%) as white solids.

### Example 35

### 4-Chloro-7-[4-(4-{4-[(4-{2-[(3R*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile [Absolute Stereochemistry not Yet Confirmed]

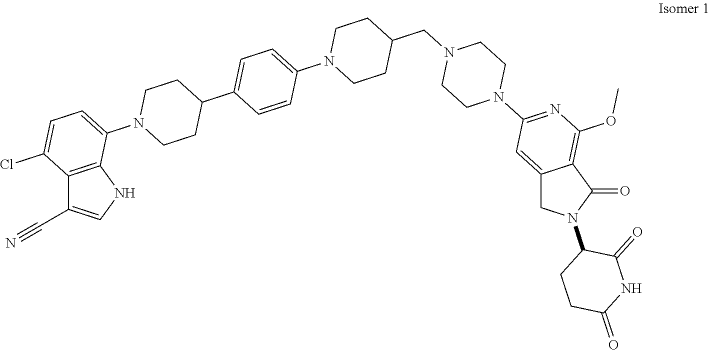

¹H NMR δ 1.19-1.29 (2H, m), 1.67 (1H, s), 1.77-1.91 (4H, m), 1.88-2.03 (3H, m), 2.20 (2H, d), 2.31 (1H, dd), 2.44 (4H, s), 2.59 (4H, q), 2.76 (2H, t), 2.84-2.95 (1H, m), 3.41 (2H, d), 3.63 (6H, d), 3.89 (3H, s), 4.13 (1H, d), 4.27 (1H, d), 4.97 (1H, dd), 6.48 (1H, s), 6.79-6.96 (3H, m), 7.09-7.2 (3H, m), 8.34 (1H, s), 10.94 (1H, s), 12.28 (1H, s); m/z: ES⁺ [M+H]⁺=790.5; >99% cc.

### Example 36

### 4-Chloro-7-[4-(4-{4-[(4-{2-[(3S*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile [Absolute Stereochemistry not Yet Confirmed]

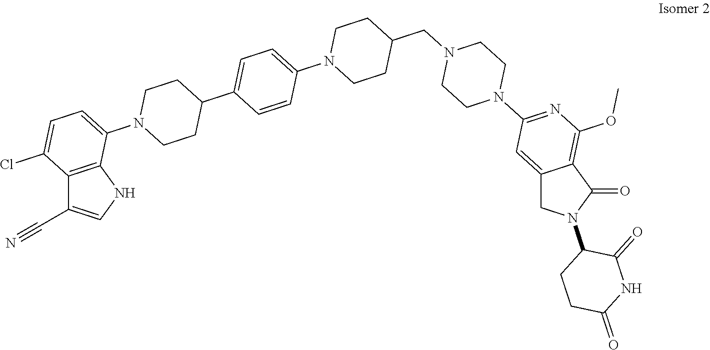

¹H NMR δ 1.19-1.29 (2H, m), 1.67 (1H, s), 1.77-1.91 (4H, m), 1.88-2.03 (3H, m), 2.20 (2H, d), 2.31 (1H, dd), 2.44 (4H, s), 2.59 (4H, q), 2.76 (2H, t), 2.84-2.95 (1H, m), 3.41 (2H, d), 3.63 (6H, d), 3.89 (3H, s), 4.13 (1H, d), 4.27 (1H, d), 4.97 (1H, dd), 6.48 (1H, s), 6.79-6.96 (3H, m), 7.09-7.2 (3H, m), 8.34 (1H, s), 10.94 (1H, s), 12.28 (1H, s); m/z: ES⁺ [M+H]⁺=790.4; >99% ee.

### Example 37

### 7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indole-3-carbonitrile

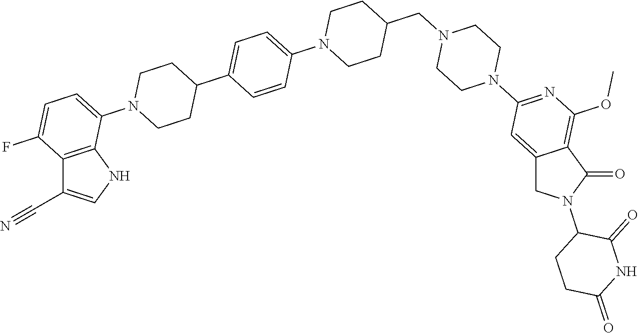 [SMILES: COc1nc(N2CCN(CC3CCN(c4ccc(C5CCN(c6ccc(F)c7c(C#N)c[nH]c67)CC5)cc4)CC3)CC2)cc2c1C(=O)N(C1CCC(=O)NC1=O)C2]

Intermediate 2c was reacted with intermediate 30e using the general synthetic method illustrated by example 24 to give the title compound (37.0 mg, 19%) as a white solid. ¹H NMR δ 1.16-1.3 (2H, m), 1.69 (1H, s), 1.83 (4H, d), 1.86-2.02 (3H, m), 2.21 (2H, d), 2.24-2.37 (1H, m), 2.45 (4H, t), 2.53-2.6 (4H, m), 2.75 (2H, t), 2.82-2.96 (1H, m), 3.37 (2H, d), 3.58-3.68 (6H, m), 3.89 (3H, s), 4.13 (1H, d), 4.27 (1H, d), 4.96 (1H, dd), 6.48 (1H, s), 6.82 (1H, dd), 6.87-6.97 (3H, m), 7.13 (2H, d), 8.27 (1H, s), 10.92 (1H, s), 12.26 (1H, s); m/z: ES⁺ [M+H]⁺=774.4.

### Example 38

### 4-Chloro-7-[(3S)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile

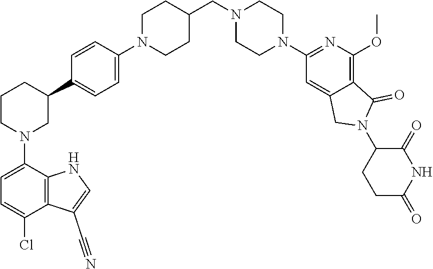 [SMILES: COc1nc(N2CCN(CC3CCN(c4ccc([C@@H]5CCCN(c6ccc(Cl)c7c(C#N)c[nH]c67)C5)cc4)CC3)CC2)cc2c1C(=O)N(C1CCC(=O)NC1=O)C2]

Intermediate 11c was reacted with intermediate 30e using the general synthetic method illustrated by example 24 to give the title compound (70.0 mg, 64%) as a white solid. ¹H NMR δ 1.21 (2H, q), 1.55 (1H, q), 1.68 (1H, s), 1.82 (2H, d), 1.89-1.94 (4H, m), 2.21 (2H, s), 2.23-2.35 (1H, m), 2.45 (3H, s), 2.54 (1H, s), 2.56-2.73 (5H, m), 2.82-2.95 (1H, m), 2.99 (1H, t), 3.30 (1H, s), 3.33 (1H, s), 3.54-3.68 (6H, m), 3.88 (3H, s), 4.13 (1H, d), 4.27 (1H, d), 4.96 (1H, dd), 6.48 (1H, s), 6.85 (3H, dd), 7.14 (3H, dd), 8.31 (1H, d), 10.92 (1H, s), 12.29 (1H, s); m/z: ES⁺ [M+H]⁺=790.4.

### Examples 39 & 40

The enantiomers of Example 38 were separated by SFC purification on a Sepiatec 100 SFC using SFC conditions Regis (R,R) Whelk-01, 21.1×250 mm, 5 micron, 50% MeOH/MeCN/50% scCO2 at a flow rate of 60 m/min, 40° C. to give, in order of elution, example 39 (isomer 1, 6.6 mg, 22%) and example 40 (isomer 2, 5.7 mg, 19%) as white solids.

### Example 39

### 4-Chloro-7-[(3S)-3-(4-{4-[(4-{2-[(3R*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile [Absolute Stereochemistry not Yet Confirmed]

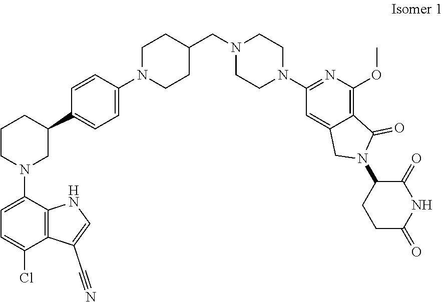

¹H NMR δ 1.13-1.3 (2H, m), 1.49-1.63 (1H, m), 1.62-1.74 (1H, m), 1.73-1.85 (2H, m), 1.85-1.98 (4H, m), 2.21 (2H, d), 2.24-2.31 (1H, m), 2.45 (4H, d), 2.53-2.76 (5H, m), 2.82-2.95 (1H, m), 2.95-3.04 (1H, m), 3.33-3.43 (2H, m), 3.54-3.69 (6H, m), 3.88 (3H, s), 4.08-4.33 (2H, m), 4.95 (1H, dd), 6.48 (1H, s), 6.77-6.96 (3H, m), 7.04-7.22 (3H, m), 8.29 (1H, s), 10.90 (1H, s), 12.27 (1H, s); m/z: ES⁺ [M+H]⁺=790.5; >99% cc.

### Example 40

### 4-Chloro-7-[(3S)-3-(4-{4-[(4-{2-[(3S*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile [Absolute Stereochemistry not Yet Confirmed]

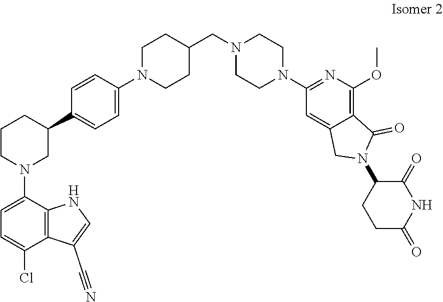

¹H NMR δ 1.15-1.3 (2H, m), 1.5-1.62 (1H, m), 1.62-1.74 (1H, m), 1.75-1.85 (2H, m), 1.84-1.97 (4H, m), 2.21 (2H, d), 2.23-2.3 (1H, m), 2.4-2.47 (4H, m), 2.52-2.66 (4H, m), 2.68-2.74 (1H, m), 2.83-2.94 (1H, m), 2.94-3.05 (1H, m), 3.31-3.4 (2H, m), 3.56-3.67 (6H, m), 3.88 (3H, s), 4.1-4.31 (2H, m), 4.95 (1H, dd), 6.48 (1H, s), 6.77-6.93 (3H, m), 7.09-7.21 (3H, m), 8.30 (1H, d), 10.90 (1H, s), 12.26 (1H, d); m/z: ES⁺ [M+H]⁺=790.5; >99% ee.

### Example 41

### Intermediate 41a: Benzyl 4-(4-bromo-2-fluorophenyl)-3,6-dihydropyridine-1(2H)-carboxylate

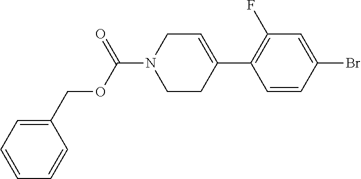 [SMILES: O=C(OCc1ccccc1)N1CC=C(c2ccc(Br)cc2F)CC1]

4-Bromo-2-fluoro-1-iodobenzene (1.00 g, 3.32 mmol) and benzyl 4-(4,4,5,5-tetramethyl-1,3,2-dioxaborolan-2-yl)-3,6-dihydropyridine-1(2H)-carboxylate (1.36 g, 3.99 mmol) were added to toluene:EtOH (2:1, 15 mL). Na₂CO₃ (2M, 5 mL, 9.97 mmol) was added and the mixture was degassed with nitrogen. Pd(dppf)Cl₂ (0.271 g, 0.33 mmol) was added and the mixture was stirred at 100° C. for 1 h. The reaction mixture cooled to RT, diluted with water (25 mL), extracted with EtOAc (60 mL) and the organic layer was washed with NaCl solution (30 mL). The organics were dried with MgSO₄, filtered and evaporated to afford crude product. The crude product was purified by flash silica chromatography, elution gradient 0 to 10% EtOAc in heptane to give the title compound (0.988 g, 76%) as a pale brown gum. ¹H NMR (CDCl₃) δ 2.49 (2H, s), 3.69 (2H, t), 4.14 (2H, q), 5.18 (2H, s), 5.92 (1H, s), 7.01-7.16 (1H, m), 7.19-7.26 (2H, m), 7.29-7.43 (5H, m).

### Intermediate 41b

### Benzyl 4-{4-[4-(dibutoxymethyl)piperidin-1-yl]-2-fluorophenyl}-3,6-dihydropyridine-1(2H)-carboxylate

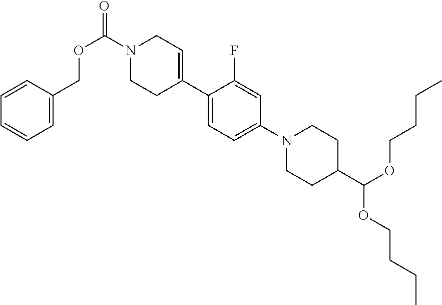 [SMILES: CCCCOC(OCCCC)C1CCN(c2ccc(C3=CCN(C(=O)OCc4ccccc4)CC3)c(F)c2)CC1]

Benzyl 4-(4-bromo-2-fluorophenyl)-3,6-dihydropyridine-1(2H)-carboxylate (1.06 g, 2.73 mmol), 4-(dibutoxymethyl)piperidine (0.785 g, 2.87 mmol) and sodium tert-butoxide (0.788 g, 8.20 mmol) were added to 1,4-dioxane (15.0 mL) and was degassed with nitrogen for 5 minutes, RuPhos (0.128 g, 0.27 mmol) and RuPhos Pd G3 (0.229 g, 0.27 mmol) were then added and the reaction was stirred at 80° C. for 2 h. The reaction was then cooled to RT and the solvent was evaporated. The residue was suspended in DCM (10 mL), filtered and the filtrate was purified by flash silica chromatography, elution gradient 0 to 25% EtOAc in heptane to give the title compound (0.349 g, 23.09%) as a yellow gum. ¹H NMR (CDCl₃) δ 0.93 (6H, t), 1.40 (5H, m), 1.5-1.65 (4H, m), 1.7-1.97 (3H, m), 2.50 (2H, s), 2.69 (2H, td), 3.44 (2H, dt), 3.55-3.84 (5H, m), 4.03-4.26 (3H, m), 5.18 (2H, d), 5.85 (1H, s), 6.46-6.73 (2H, m), 7.03-7.16 (1H, m), 7.27-7.5 (5H, m); m/z: ES+[M+H]⁺=553.0.

### Intermediate 41c

### 4-(Dibutoxymethyl)-1-[3-fluoro-4-(piperidin-4-yl)phenyl]piperidine

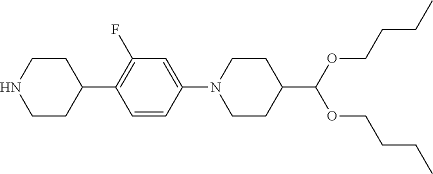 [SMILES: CCCCOC(OCCCC)C1CCN(c2ccc(C3CCNCC3)c(F)c2)CC1]

Benzyl 4-{4-[4-(dibutoxymethyl)piperidin-1-yl]-2-fluorophenyl}-3,6-dihydropyridine-1(2H)-carboxylate (2.39 g, 4.31 mmol) was dissolved in EtOH (25 mL) and Pd/C (10%, 0.458 g, 4.31 mmol) was added. The reaction was stirred under an atmosphere of hydrogen at 2 bar pressure for 4 h at RT. The catalyst was filtered off through a pad of Celite® and evaporated to dryness to give the title compound (1.70 g, 94%) as a pale yellow solid which was used without further purification. ¹H NMR (CDCl₃) δ 0.93 (6H, t), 1.33-1.49 (6H, m), 1.5-1.61 (4H, m), 1.62-1.9 (7H, m), 2.64 (2H, td), 2.7-2.81 (2H, m), 2.86 (1H, m), 3.19 (2H, dd), 3.43 (2H, dt), 3.62 (4H, dt), 4.18 (1H, d), 6.57 (1H, dd), 6.65 (1H, dd), 7.04-7.1 (1H, m).

### Intermediate 41d

### 7-(4-{4-[4-(Dibutoxymethyl)piperidin-1-yl]-2-fluorophenyl}piperidin-1-yl)-4-methyl-1H-indole-3-carbonitrile

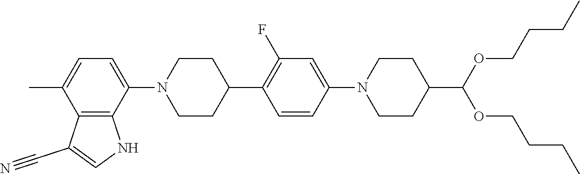 [SMILES: CCCCOC(OCCCC)C1CCN(c2ccc(C3CCN(c4ccc(C)c5c(C#N)c[nH]c45)CC3)c(F)c2)CC1]

The intermediate was prepared using methodology described in intermediate 11c using intermediate 8a and 4-(dibutoxymethyl)-1-[3-fluoro-4-(piperidin-4-yl)phenyl]piperidine to give the title compound (22 mg, 4.5%) as a white solid. ¹H NMR (CDCl₃) δ 0.93 (6H, t), 1.33-1.5 (6H, m), 1.52-1.62 (4H, m), 1.69-1.82 (1H, m), 1.87 (2H, d), 1.93-2.05 (4H, m), 2.67 (2H, td), 2.72-2.75 (3H, m), 2.86-3 (3H, m), 3.36 (2H, d), 3.44 (2H, dt), 3.57-3.71 (4H, m), 4.19 (1H, d), 6.61 (1H, dd), 6.68 (1H, dd), 6.90 (1H, d), 6.95 (1H, dd), 7.12 (1H, t), 7.70 (1H, s), 8.74-8.82 (1H, m); m/z: ES⁺ [M+H]⁺=575.6.

### Example 41

### 7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]-2-fluorophenyl}piperidin-1-yl)-4-methyl-1H-indole-3-carbonitrile

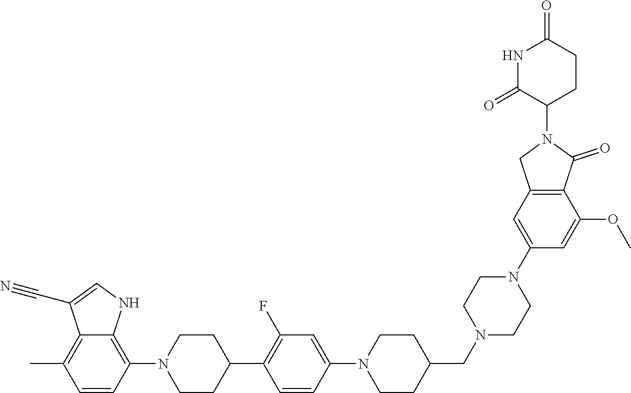 [SMILES: COc1cc(N2CCN(CC3CCN(c4ccc(C5CCN(c6ccc(C)c7c(C#N)c[nH]c67)CC5)c(F)c4)CC3)CC2)cc2c1C(=O)N(C1CCC(=O)NC1=O)C2]

The title compound was prepared using methodology described in example 23 using 7-(4-{4-[4-(dibutoxymethyl)piperidin-1-yl]-2-fluorophenyl}piperidin-1-yl)-4-methyl-1H-indole-3-carbonitrile and intermediate 7c to give the title compound in the form of a formate salt (15 mg, 53.3%) as a white solid. ¹H NMR δ 1.12-1.35 (3H, m), 1.67-1.87 (5H, m), 1.89-1.96 (1H, m), 1.98-2.12 (3H, m), 2.17-2.38 (5H, m), 2.45-2.57 (5H, m), 2.58-2.63 (3H, m), 2.64-2.98 (6H, m), 3.39 (2H, d), 3.70 (2H, d), 3.84 (3H, s), 4.11 (1H, d), 4.24 (1H, d), 4.97 (1H, dd), 6.50 (1H, s), 6.62 (1H, s), 6.67-6.81 (3H, m), 6.88 (1H, d), 7.16 (1H, t), 8.17 (1H, s), 10.90 (1H, s), 11.91 (1H, s); m/z: ES⁺ [M+H]⁺=787.5.

### Example 42

### 7-(4-{4-[4-({4-[4-(2,4-Dioxo-1,3-diazinan-1-yl)-1H-indol-1-yl]piperidin-1-yl}methyl)piperidin-1-yl]-2-fluorophenyl}piperidin-1-yl)-4-methyl-1H-indole-3-carbonitrile

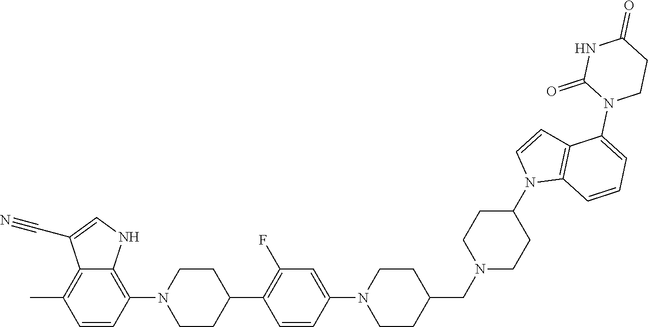 [SMILES: Cc1ccc(N2CCC(c3ccc(N4CCC(CN5CCC(n6ccc7c(N8CCC(=O)NC8=O)cccc76)CC5)CC4)cc3F)CC2)c2[nH]cc(C#N)c12]

The title compound was prepared using methodology described in example 24 using intermediate 41d and intermediate 5f to give the title compound (17 mg, 44%) as a white solid. ¹H NMR δ 1.13-1.32 (2H, m), 1.62-1.76 (1H, m), 1.82 (4H, t), 1.91-2.12 (6H, m), 2.14-2.29 (4H, m), 2.57-2.62 (3H, m), 2.64-2.91 (7H, m), 3.01 (2H, d), 3.42 (2H, d), 3.71 (2H, d), 3.79 (2H, t), 4.33-4.48 (1H, m), 6.42 (1H, d), 6.67-6.81 (3H, m), 6.87 (1H, d), 6.97 (1H, dd), 7.16 (2H, td), 7.52 (1H, d), 7.55 (1H, d), 8.15 (1H, s), 10.32 (1H, s), 11.71-12.17 (1H, m); m/z: ES⁺ [M+H]⁺=741.5.

### Example 43

### Intermediate 43a

### 5-Bromo-2,3-dimethylphenol

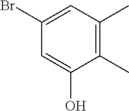

To a stirred solution of 5-bromo-2,3-dimethylaniline (3.0 g, 14.99 mmol) and H₂SO₄ (4.0 ml, 75.0 mmol) in water (24 mL) was added sodium nitrite (1.2 g, 17.39 mmol) at 0° C. The reaction mixture was stirred at 0° C. for 1 h then stirred to 50° C. for 5 h. The reaction mixture was diluted with water (50 mL) and extracted with EtOAc (200 mL). The organic layer was dried over Na₂SO₄, filtered and evaporated to afford crude product. The crude product was purified by flash silica chromatography, elution gradient 5 to 7% EtOAc in heptane to give the title compound (1.5 g, 41.7%) as a pale brown solid. ¹H NMR (CDCl₃) δ 2.12 (3H, s), 2.26 (3H, s), 4.84 (1H, br s), 6.82 (1H, s), 6.93 (1H, s); m/z: ES⁻ [M−H]⁻=199.0.

### Intermediate 43b

### 5-Bromo-1-methoxy-2,3-dimethylbenzene

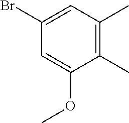

To a stirred solution of 5-bromo-2,3-dimethylphenol (1.5 g, 7.46 mmol) in acetone (12 mL) was added K₂CO₃(2.06 g, 14.92 mmol) and methyl iodide (0.46 mL, 7.46 mmol) at RT. The reaction mixture was stirred at 70° C. for 6 h. The reaction mixture was evaporated under vacuum, diluted with water (50 mL) and extracted with EtOAc (150 mL). The organic layer was washed with NaCl solution (30 mL), dried over Na₂SO₄, filtered and evaporated to dryness to afford crude product. The crude product was flash silica chromatography, elution gradient 1 to 3% EtOAc in heptane to give the title compound (1.21 g, 74.3%) as a pale brown liquid. ¹H NMR (CDCl₃) δ 2.10 (3H, s), 2.26 (3H, s), 3.82 (3H, s), 6.86 (1H, s), 6.95 (1H, s).

### Intermediate 43c

### 5-Bromo-3-methoxybenzene-1,2-dicarboxylic acid

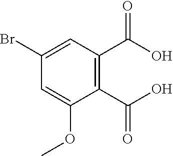

To a stirred solution of 5-bromo-1-methoxy-2,3-dimethylbenzene (1.2 g, 5.58 mmol) in tert-BuOH (5 mL) and water (20 mL) was added KMnO₄ (5.73 g, 36.3 mmol) at RT. The reaction mixture was stirred at 110° C. for 24 h. The reaction mixture was filtered over Celite® and washed with water (80 mL). The filtrate was acidified to pH 1 with HCl and the product was extracted with EtOAc (200 mL). The organic layer was dried over Na₂SO₄, filtered and evaporated to dryness to give the title compound (1.4 g, 83%) as a white solid. ¹H NMR δ 3.85 (3H, s), 7.55 (1H, s), 7.58 (1H, d), 13.36 (2H, s); m/z: ES⁺ [M+H]⁺=273.0.

### Intermediate 43d

### Dimethyl 5-bromo-3-methoxybenzene-1,2-dicarboxylate

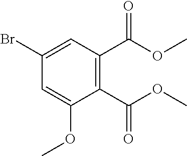 [SMILES: COC(=O)c1cc(Br)cc(OC)c1C(=O)OC]

To a stirred solution of 5-bromo-3-methoxybenzene-1,2-dicarboxylic acid (1.3 g, 4.73 mmol) in MeOH (30 mL) was added H₂SO₄ (5.0 ml, 94 mmol) at 0° C. The reaction mixture stirred at 70° C. for 24 h. The reaction mixture was evaporated and diluted with water (50 mL), basified to pH8 with Na₂CO₃ (2M), extracted with EtOAc (200 mL). The organic layer was dried over Na₂SO₄, filtered and evaporated to dryness to give the title compound (1.4 g, 92%). ¹H NMR (CDCl₃) δ 3.88 (3H, s), 3.91 (3H, s), 3.96 (3H, s), 7.27 (1H, d), 7.77 (1H, d). no mass ion

### Intermediate 43e

### Dimethyl 5-[4-(tert-butoxycarbonyl)piperazin-1-yl]-3-methoxybenzene-1,2-dicarboxylate

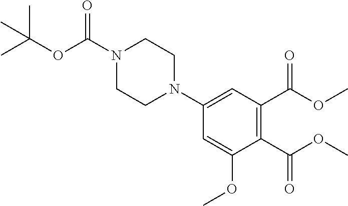 [SMILES: COC(=O)c1cc(N2CCN(C(=O)OC(C)(C)C)CC2)cc(OC)c1C(=O)OC]

To a stirred solution of Dimethyl 5-bromo-3-methoxybenzene-1,2-dicarboxylate (1.2 g, 3.96 mmol) in toluene (10 mL) was added Cs₂CO₃ (3.87 g, 11.88 mmol), tert-butyl piperazine-1-carboxylate (1.10 g, 5.94 mmol) and RuPhos (0.37 g, 0.436 mmol). The reaction mixture was degassed with nitrogen for 15 mins and RuPhos Pd G3 (0.310 mg, 1.321 mmol) was added to the reaction mixture followed by stirring at 100° C. for 12 h. The reaction mixture was diluted with water (100 mL) and extracted with EtOAc (300 mL). The organic layer was washed with NaCl solution (30 mL), dried over Na₂SO₄, filtered and evaporated to dryness afford crude product. The crude product was triturated with hexane (100 mL) to give the title compound (1.05 g, 48.7%) as a pale brown liquid. ¹H NMR (CDCl₃) δ 1.51 (9H, s), 3.25 (3H, s), 3.60 (3H, s), 3.86-3.94 (9H, m), 6.60 (1H, s), 7.07 (1H, s); m/z: ES⁺ [M+H]⁺=409.2.

### Intermediate 43f

### 5-[4-(tert-Butoxycarbonyl)piperazin-1-yl]-3-methoxybenzene-1,2-dicarboxylic acid

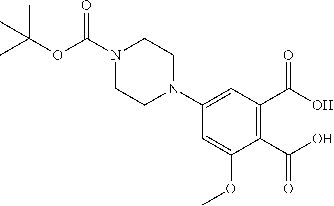 [SMILES: COc1cc(N2CCN(C(=O)OC(C)(C)C)CC2)cc(C(=O)O)c1C(=O)O]

To a stirred solution of dimethyl 5-[4-(tert-butoxycarbonyl)piperazin-1-yl]-3-methoxybenzene-1,2-dicarboxylate (1.0 g, 2.448 mmol) in MeOH (5 mL), THF (5 mL) and water (5 mL) was added NaOH (0.294 g, 7.34 mmol) at RT and stirred for 72 h. The mixture was evaporated under vacuum, diluted with water (50 mL) and acidified with citric acid (1M) to pH3. The product was extracted with EtOAc (300 mL), dried over Na₂SO₄, filtered and evaporated under vacuum to give the title compound (900 mg, 70.7%) as a pale yellow solid. ¹H NMR δ 1.41-1.44 (9H, m), 3.18-3.25 (4H, m), 3.44-3.49 (4H, m), 3.80 (3H, s), 6.78 (1H, d), 6.91 (1H, d); m/z: ES⁻ [M−H]⁻=379.2.

### Intermediate 43g

### tert-Butyl 4-[2-(2,6-dioxopiperidin-3-yl)-7-methoxy-1,3-dioxo-2,3-dihydro-1H-isoindol-5-yl]piperazine-1-carboxylate

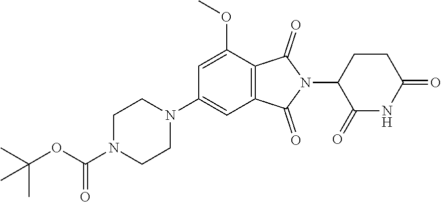 [SMILES: COc1cc(N2CCN(C(=O)OC(C)(C)C)CC2)cc2c1C(=O)N(C1CCC(=O)NC1=O)C2=O]

A stirred solution of 5-[4-(tert-butoxycarbonyl)piperazin-1-yl]-3-methoxybenzene-1,2-dicarboxylic acid (900 mg, 2.366 mmol) and 3-aminopiperidine-2,6-dione hydrochloride (506 mg, 3.08 mmol) in pyridine (10 mL) was heated to 120° C. for 4 h. The reaction mixture was cooled to RT and diluted with water (70 mL) and extracted with EtOAc (150 mL). The organic layer was dried over Na₂SO₄, filtered and evaporated to dryness to afford crude product. The crude product was purified by flash silica chromatography, elution gradient 1 to 2% MeOH in DCM to give the title compound (580 mg, 48.1%) as a pale yellow solid. ¹H NMR δ 1.21-1.28 (2H, m), 1.43 (9H, s), 1.93-2.03 (1H, m), 2.54-2.62 (2H, m), 2.81-2.94 (1H, m), 3.49 (8H, d), 3.93 (3H, s), 5.01 (1H, dd), 6.68 (1H, s), 6.97 (1H, s), 11.05 (1H, s). no mass ion.

### Example 43

### 7-(4-{4-[4-({4-[2-(2,6-Dioxopiperidin-3-yl)-7-methoxy-1,3-dioxo-2,3-dihydro-1H-isoindol-5-yl]piperazin-1-yl}methyl)piperidin-1-yl]-2-fluorophenyl}piperidin-1-yl)-4-methyl-1H-indole-3-carbonitrile

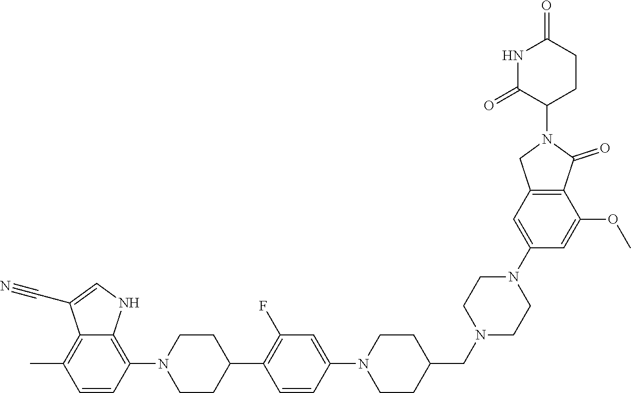 [SMILES: COc1cc(N2CCN(CC3CCN(c4ccc(C5CCN(c6ccc(C)c7c(C#N)c[nH]c67)CC5)c(F)c4)CC3)CC2)cc2c1C(=O)N(C1CCC(=O)NC1=O)C2]

The title compound was prepared using methodology described in example 23 using intermediate 41d and tert-butyl 4-[2-(2,6-dioxopiperidin-3-yl)-7-methoxy-1,3-dioxo-2,3-dihydro-1H-isoindol-5-yl]piperazine-1-carboxylate to give the title compound in the form of a formate salt (17 mg, 44%) as a white solid. ¹H NMR δ 1.15-1.31 (2H, m), 1.67-1.87 (5H, m), 1.93-2.1 (4H, m), 2.24 (2H, d), 2.45-2.54 (4H, m), 2.58-2.64 (3H, m), 2.64-2.96 (6H, m), 3.36-3.43 (3H, m), 3.45-3.53 (4H, m), 3.71 (2H, d), 3.93 (3H, s), 5.01 (1H, dd), 6.66-6.82 (4H, m), 6.88 (1H, d), 6.97 (1H, s), 7.17 (1H, t), 8.17 (1H, d), 11.04 (1H, s), 11.91 (1H, s); m/z: ES⁺ [M+H]⁺=801.5.

### Example 44

### 7-{4-[4-(4-{4-[2-(2,6-Dioxopiperidin-3-yl)-1-oxo-2,3-dihydro-1H-isoindol-5-yl]piperazin-1-yl}butoxy)phenyl]piperidin-1-yl}-4-fluoro-1H-indole-3-carbonitrile

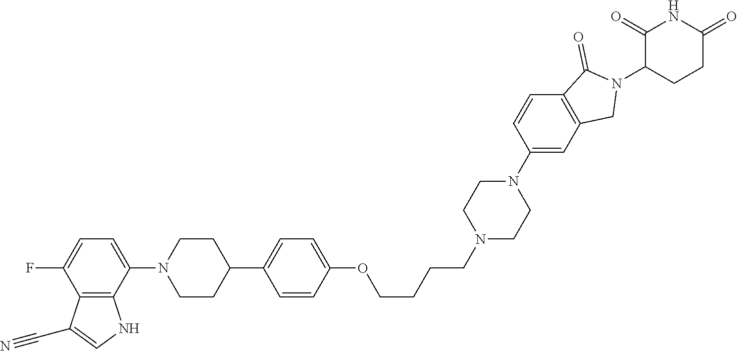 [SMILES: N#Cc1c[nH]c2c(N3CCC(c4ccc(OCCCCN5CCN(c6ccc7c(c6)CN(C6CCC(=O)NC6=O)C7=O)CC5)cc4)CC3)ccc(F)c12]

Intermediate 33a was reacted with intermediate 1i using the general synthetic method illustrated by example 24 to give the title compound (20.0 mg, 36.3%) as a white solid. ¹H NMR δ 1.60 (2H, t), 1.72 (2H, d), 1.73-1.87 (2H, m), 1.93 (3H, t), 2.20-2.41 (3H, m), 2.55 (6H, d), 2.70-2.93 (3H, m), 3.25 (4H, d), 3.36 (2H, d), 3.97 (2H, t), 4.08-4.46 (2H, m), 5.03 (1H, dd), 6.54-7.01 (4H, m), 7.04 (2H, d), 7.18 (2H, d), 7.51 (1H, d), 8.25 (1H, d), 10.92 (1H, s), 12.23 (1H, s); m/z: ES⁺ [M+H]⁺=718.4.

### Example 45

### 4-Chloro-7-{4-[4-(4-{4-[2-(2,6-dioxopiperidin-3-yl)-1-oxo-2,3-dihydro-1H-isoindol-5-yl]piperazin-1-yl}butoxy)phenylpiperidin-1-yl}-1H-indole-3-carbonitrile

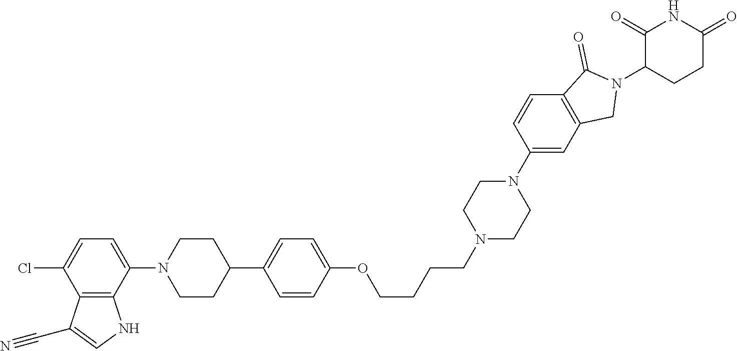 [SMILES: N#Cc1c[nH]c2c(N3CCC(c4ccc(OCCCCN5CCN(c6ccc7c(c6)CN(C6CCC(=O)NC6=O)C7=O)CC5)cc4)CC3)ccc(Cl)c12]

Intermediate 32d was reacted with intermediate 1i using the general synthetic method illustrated by example 24 to give the title compound (47.0 mg, 30%) as a white solid. ¹H NMR δ 1.45-2.05 (8H, m), 2.36 (3H, dt), 2.53 (1H, s), 2.63 (2H, d), 2.71-2.99 (3H, m), 3.28 (4H, s), 3.41 (2H, d), 3.99 (2H, t), 4.20 (1H, d), 4.33 (1H, d), 5.04 (1H, dd), 6.81-6.95 (3H, m), 7.05 (2H, d), 7.12-7.24 (3H, m), 7.52 (1H, d), 8.32 (1H, s), 10.93 (1H, s), 12.26 (1H, s) 4H under DMSO; m/z: ES⁺ [M+H]⁺=734.3.

### Example 46

### 4-Chloro-7-{4-[4-(4-{4-[4-(2,4-dioxo-1,3-diazinan-1-yl)-1H-indol-1-yl]piperidin-1-yl}butoxy)phenyl]piperidin-1-yl}-1H-indole-3-carbonitrile

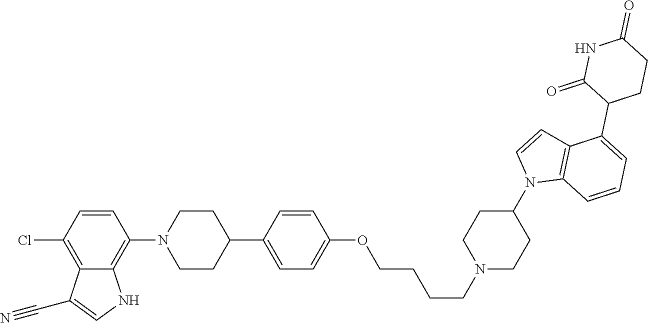 [SMILES: N#Cc1c[nH]c2c(N3CCC(c4ccc(OCCCCN5CCC(n6ccc7c(C8CCC(=O)NC8=O)cccc76)CC5)cc4)CC3)ccc(Cl)c12]

Intermediate 32d was reacted with intermediate 5f using the general synthetic method illustrated by example 23 to give the title compound in the form of a formate salt (27.0 mg, 41%) as a white solid. ¹H NMR δ 1.56-1.7 (2H, m), 1.72-1.82 (2H, m), 1.81-1.91 (2H, m), 1.91-2.05 (7H, m), 2.19 (3H, t), 2.42-2.47 (2H, m), 2.72-2.81 (3H, m), 3.05 (2H, d), 3.43 (2H, d), 3.78 (2H, t), 4.00 (2H, t), 4.28-4.55 (1H, m), 6.42 (1H, d), 6.83-7.02 (4H, m), 7.1-7.25 (4H, m), 7.45-7.6 (2H, m), 8.15 (1H, s), 8.32 (1H, s), 10.31 (1H, s); m/z: ES⁺ [M+H]⁺=718.6.

### Example 47

### 4-Chloro-7-[(3R)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile

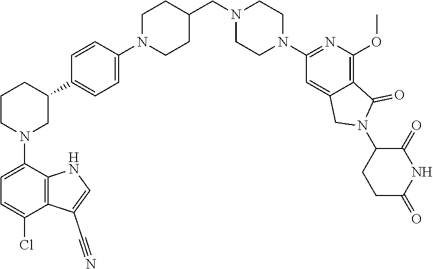 [SMILES: COc1nc(N2CCN(CC3CCN(c4ccc([C@H]5CCCN(c6ccc(Cl)c7c(C#N)c[nH]c67)C5)cc4)CC3)CC2)cc2c1C(=O)N(C1CCC(=O)NC1=O)C2]

Intermediate 21d was reacted with intermediate 30e using the general synthetic method illustrated by example 24 to give the title compound (31.0 mg, 28%) as a white solid. ¹H NMR δ 1.21 (2H, d), 1.55 (1H, q), 1.67 (1H, s), 1.75-1.93 (6H, m), 2.20 (2H, d), 2.31 (1H, tt), 2.44 (4H, s), 2.49 (1H, s), 2.51-2.74 (5H, m), 2.82-2.98 (1H, m), 3.01 (1H, d), 3.37 (1H, d), 3.60 (6H, s), 3.88 (3H, s), 4.13 (1H, d), 4.27 (1H, d), 4.96 (1H, dd), 6.48 (1H, s), 6.85 (3H, dd), 7.14 (3H, dd), 8.31 (1H, d), 10.92 (1H, s), 12.28 (1H, d); m/z: ES⁺ [M+H]⁺=790.4.

### Example 48

### Intermediate 48a

### 4-Methyl-7-{4-[4-(4-oxobutoxy)phenyl]piperidin-1-yl}-1H-indole-3-carbonitrile

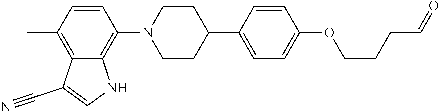 [SMILES: Cc1ccc(N2CCC(c3ccc(OCCCC=O)cc3)CC2)c2[nH]cc(C#N)c12]

Intermediate 27g (377 mg, 0.85 mmol) was stirred at 60° C. in formic acid (5 mL) for 1 h. The mixture was evaporated, diluted with DCM (50 mL), washed with NaHCO₃ solution (20 mL) and the organic layer was dried over a phase separating cartridge. The solvent was evaporated to give the title compound (0.377 g, 111%) which was used without further purification. ¹H NMR (CDCl₃) δ 1.84-2.03 (4H, m), 2.07-2.18 (2H, m), 2.6-2.7 (3H, m), 2.71-2.75 (3H, m), 2.89 (2H, td), 3.31-3.41 (2H, m), 4.01 (2H, t), 6.84-6.88 (2H, m), 6.90 (1H, d), 6.96 (1H, dd), 7.17-7.22 (2H, m), 7.71 (1H, d), 8.75 (1H, s), 9.85 (1H, t); m/z: ES⁺ [M+H]⁺=402.3.

### Example 48

### 7-{4-[4-(4-{4-[2-(2,6-Dioxopiperidin-3-yl)-7-methoxy-1,3-dioxo-2,3-dihydro-1H-isoindol-5-yl]piperazin-1-yl}butoxy)phenyl]piperidin-1-yl}-4-methyl-1H-indole-3-carbonitrile

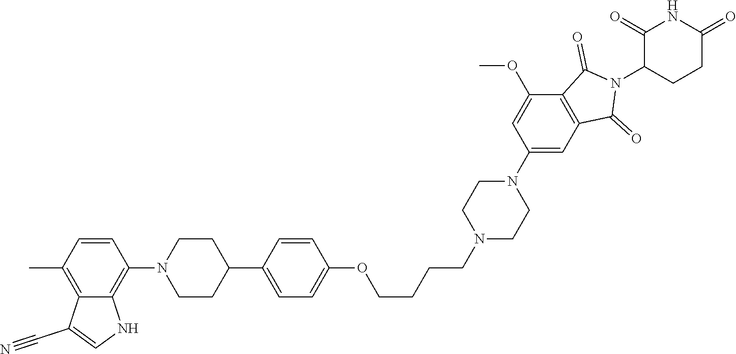 [SMILES: COc1cc(N2CCN(CCCCOc3ccc(C4CCN(c5ccc(C)c6c(C#N)c[nH]c56)CC4)cc3)CC2)cc2c1C(=O)N(C1CCC(=O)NC1=O)C2=O]

Intermediate 43g (133 mg, 0.28 mmol) was stirred in formic acid (2 mL) at 60° C. for 1 h. The mixture was evaporated to dryness and the residue was dissolved in NMP (1 mL) at RT followed by the addition of 4-methyl-7-{4-[4-(4-oxobutoxy)phenyl]piperidin-1-yl}-1H-indole-3-carbonitrile (94 mg, 0.23 mmol). Sodium triacetoxyborohydride (124 mg, 0.59 mmol) was added after 5 minutes and the resulting suspension was stirred at RT for 20 mins. Purification of the reaction mixture by preparative HPLC (Column A, Eluent A) gave the title compound in the form of a formate salt (0.061 g, 34.4%) as a yellow solid. ¹H NMR δ 1.56-1.69 (2H, m), 1.7-1.81 (2H, m), 1.8-1.88 (2H, m), 1.98 (3H, q), 2.37-2.46 (3H, m), 2.49-2.56 (4H, m), 2.58-2.64 (4H, m), 2.72-2.8 (2H, m), 2.8-2.96 (1H, m), 3.35-3.43 (3H, m), 3.43-3.52 (4H, m), 3.93 (3H, s), 4.00 (2H, t), 5.00 (1H, dd), 6.68 (1H, d), 6.78 (1H, d), 6.85-6.93 (3H, m), 6.96 (1H, d), 7.21 (2H, d), 8.15 (1H, s), 8.17 (1H, d), 11.04 (1H, s); m/z: ES⁺ [M+H]⁺=758.5.

### Example 49

### 4-Chloro-7-(4-{4-[4-({4-[2-(2,6-dioxopiperidin-3-yl)-7-methoxy-1,3-dioxo-2,3-dihydro-1H-isoindol-5-yl]piperazin-1-yl}methyl)piperidin-1-yl]phenyl}piperidin-1-yl)-1H-indole-3-carbonitrile

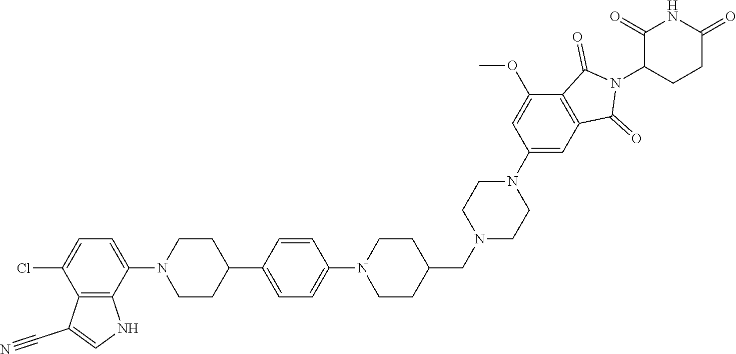 [SMILES: COc1cc(N2CCN(CC3CCN(c4ccc(C5CCN(c6ccc(Cl)c7c(C#N)c[nH]c67)CC5)cc4)CC3)CC2)cc2c1C(=O)N(C1CCC(=O)NC1=O)C2=O]

The title compound was prepared using methodology described in example 23 using intermediate 1f and intermediate 43g to give the title compound in the form of a formate salt (28 mg, 31%) as a yellow solid. ¹H NMR δ 1.22-1.4 (3H, m), 1.71-1.82 (1H, m), 1.85-1.96 (4H, m), 1.96-2.1 (4H, m), 2.30 (2H, d), 2.65-2.71 (3H, m), 2.79-3.02 (7H, m), 3.45-3.59 (6H, m), 3.71 (2H, d), 3.99 (3H, s), 5.06 (1H, dd), 6.66-6.81 (1H, m), 6.88-7.1 (4H, m), 7.15-7.28 (3H, m), 8.22 (1H, s), 8.38 (1H, s), 11.10 (1H, s); m/z: ES⁺ [M+H]⁺=803.6.

### Example 50

### Intermediate 50a

### 2-(2,6-Dioxopiperidin-3-yl)-5,6-difluoroisoindoline-1,3-dione

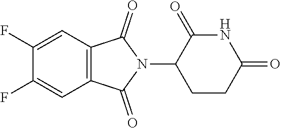 [SMILES: O=C1CCC(N2C(=O)c3cc(F)c(F)cc3C2=O)C(=O)N1]

Potassium acetate (5.04 g, 51.37 mmol) was added in one portion to 3-aminopiperidine-2,6-dione hydrochloride salt (3 g, 18.23 mmol) and 4,5-difluorophthalic acid (3.35 g, 16.57 mmol) in AcOH (30 mL) at RT. The resulting solution was stirred at 90° C. for 18 h. The mixture was then cooled to RT and a solid formed. Water (30 mL) was added. The solid was collected by filtration, washed with water (2×5 mL) and dried at 45° C. under vacuum overnight to give the title compound (3.43 g, 70%) as a dark grey solid; ¹H NMR δ 2.01-2.12 (1H, m), 2.53-2.7 (2H, m), 2.83-2.95 (1H, m), 5.17 (1H, dd), 8.15 (2H, t), 11.13 (1H, s); m/z: ES⁻ [M−H]-293.

### Intermediate 50b

### tert-Butyl 4-[2-(2,6-dioxo-3-piperidyl)-6-fluoro-1,3-dioxo-isoindolin-5-yl]piperazine-1-carboxylate

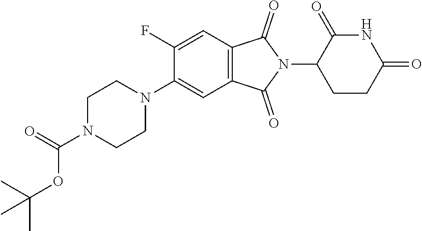 [SMILES: CC(C)(C)OC(=O)N1CCN(c2cc3c(cc2F)C(=O)N(C2CCC(=O)NC2=O)C3=O)CC1]

DMSO (0.5 mL) and then DIPEA (118 μL, 0.68 mmol) were added to a mixture of 2-(2,6-dioxopiperidin-3-yl)-5,6-difluoroisoindoline-1,3-dione (100 mg, 0.34 mmol) and tert-butyl piperazine-1-carboxylate (69.6 mg, 0.37 mmol). This mixture was then heated at 100° C. for 2h. Sat. NH₄Cl solution (0.5 mL) and water (1.5 mL) were then added. A brown precipitate was collected by filtration, washed with water and dried in a vacuum oven to give the title compounds (86 mg, 55%) as dark brown solid; ¹H NMR δ 1.44 (9H, s), 2.04 (1H, dq), 2.55-2.62 (1H, m), 2.89 (1H, m), 3.09 (1H, d), 3.16-3.26 (4H, m), 3.45-3.55 (4H, m), 5.11 (1H, dd), 7.49 (1H, d), 7.75 (1H, d), 11.09 (1H, s); m/z: ES⁻ [M−H]- 459.

### Example 50

### 7-{4-[4-(4-{4-[2-(2,6-Dioxopiperidin-3-yl)-6-fluoro-1,3-dioxo-2,3-dihydro-1H-isoindol-5-yl]piperazin-1-yl}butoxy)phenyl]piperidin-1-yl}-4-methyl-1H-indole-3-carbonitrile

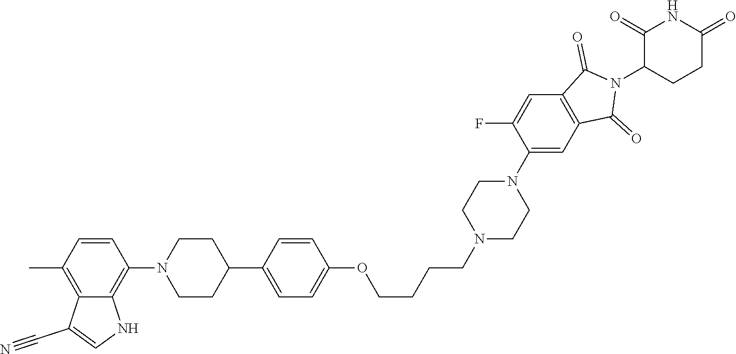 [SMILES: Cc1ccc(N2CCC(c3ccc(OCCCCN4CCN(c5cc6c(cc5F)C(=O)N(C5CCC(=O)NC5=O)C6=O)CC4)cc3)CC2)c2[nH]cc(C#N)c12]

The title compound was prepared using methodology described in example 48 using tert-butyl 4-[2-(2,6-dioxo-3-piperidyl)-6-fluoro-1,3-dioxo-isoindolin-5-yl]piperazine-1-carboxylate and intermediate 48a to give the title compound in the form of a formate salt (95 mg, 54.4%) as a white solid. ¹H NMR (CDCl₃) δ 1.71-1.88 (4H, m), 1.89-2.03 (4H, m), 2.08-2.16 (1H, m), 2.5-2.95 (15H, m), 3.26-3.37 (4H, m), 3.42 (2H, d), 3.99 (2H, t), 4.92 (1H, dd), 6.84-6.92 (3H, m), 6.93-6.97 (1H, m), 7.19 (2H, d), 7.38 (1H, d), 7.46 (1H, d), 7.70 (1H, s), 8.77 (1H, s), 9.25 (1H, s); m/z: ES⁺ [M+H]⁺=746.5.

### Example 51

### 4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-1H-indole-3-carbonitrile

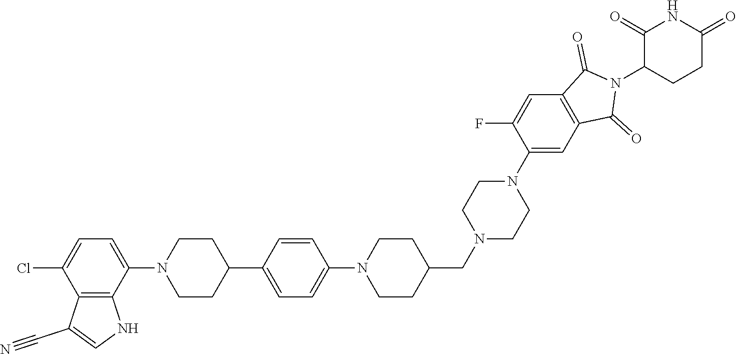 [SMILES: N#Cc1c[nH]c2c(N3CCC(c4ccc(N5CCC(CN6CCN(c7cc8c(cc7F)C(=O)N(C7CCC(=O)NC7=O)C8=O)CC6)CC5)cc4)CC3)ccc(Cl)c12]

The title compound was prepared using methodology described in example 7 using intermediate 1f and intermediate 50b to give the title compound (35 mg, 35.5%) as a yellow solid. ¹H NMR (90° C.) S 1.27-1.38 (3H, m), 1.68-1.79 (1H, m), 1.82-1.93 (4H, m), 1.95-2.04 (2H, m), 2.09-2.15 (1H, m), 2.31 (2H, d), 2.6-2.65 (4H, m), 2.71-2.79 (2H, m), 2.83-2.91 (4H, m), 3.3-3.37 (4H, m), 3.46 (2H, d), 3.65 (2H, d), 5-5.2 (1H, m), 6.67 (1H, d), 6.85-6.97 (3H, m), 7.02 (1H, d), 7.1-7.23 (3H, m), 7.46 (1H, d), 7.64 (1H, d), 8.04-8.24 (2H, m); m/z: ES⁺ [M+H]⁺=791.7.

### Example 52

### 7-(4-{4-[4-({4-[2-(2,6-Dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-4-fluoro-1H-indole-3-carbonitrile

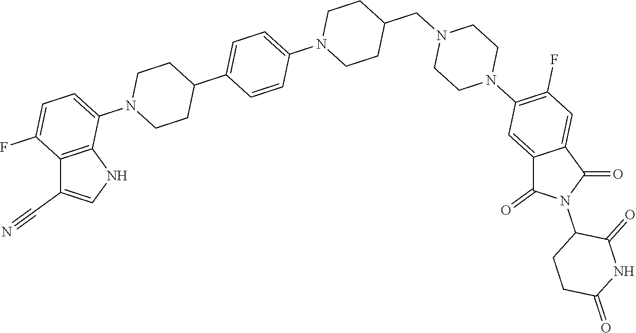 [SMILES: N#Cc1c[nH]c2c(N3CCC(c4ccc(N5CCC(CN6CCN(c7cc8c(cc7F)C(=O)N(C7CCC(=O)NC7=O)C8=O)CC6)CC5)cc4)CC3)ccc(F)c12]

The title compound was prepared using methodology described in example 7 using intermediate 2c and intermediate 50b to give the title compound (58 mg, 29.2%) as a yellow solid. ¹H NMR δ 1.24 (2H, q), 1.68 (1H, s), 1.77-1.88 (4H, m), 1.88-2.1 (3H, m), 2.24 (2H, d), 2.51-2.56 (6H, m), 2.62 (3H, t), 2.75 (2H, t), 2.82-2.96 (1H, m), 3.25 (4H, t), 3.38 (2H, d), 3.64 (2H, d), 5.11 (1H, dd), 6.82 (1H, dd), 6.87-6.97 (3H, m), 7.13 (2H, d), 7.45 (1H, d), 7.73 (1H, d), 8.27 (1H, s), 11.12 (1H, s), 12.25 (1H, s); m/z: ES⁺ [M+H]⁺=775.3.

### Example 53

### 4-Chloro-7-[(3S)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile

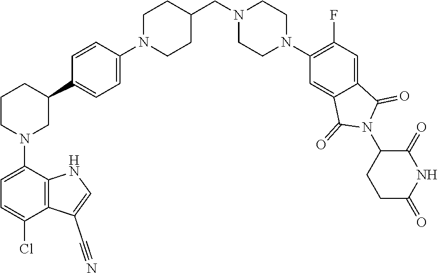 [SMILES: N#Cc1c[nH]c2c(N3CCC[C@@H](c4ccc(N5CCC(CN6CCN(c7cc8c(cc7F)C(=O)N(C7CCC(=O)NC7=O)C8=O)CC6)CC5)cc4)C3)ccc(Cl)c12]

The title compound was prepared using methodology described in example 7 using intermediate 11c and intermediate 50b to give the title compound (53 mg, 48.3%) as a yellow solid. ¹H NMR δ 1.21 (2H, q), 1.54 (1H, q), 1.67 (1H, s), 1.74-1.87 (3H, m), 1.93 (2H, d), 2.04 (1H, dd), 2.22 (2H, s), 2.49 (1H, s), 2.51-2.79 (9H, m), 2.81-3.05 (2H, m), 3.13-3.31 (5H, m), 3.33 (1H, s), 3.62 (2H, d), 5.11 (1H, dd), 6.85 (3H, dd), 7.14 (3H, dd), 7.45 (1H, d), 7.73 (1H, d), 8.31 (1H, d), 11.12 (1H, s), 12.29 (1H, s); m/z: ES⁺ [M+H]⁺=791.3.

### Example 54

### 4-Chloro-7-[(3R)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile

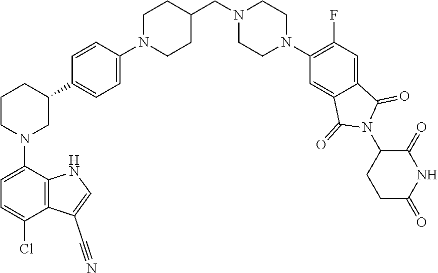 [SMILES: N#Cc1c[nH]c2c(N3CCC[C@H](c4ccc(N5CCC(CN6CCN(c7cc8c(cc7F)C(=O)N(C7CCC(=O)NC7=O)C8=O)CC6)CC5)cc4)C3)ccc(Cl)c12]

The title compound was prepared using methodology described in example 7 using intermediate 21d and intermediate 50b to give the title compound (30 mg, 27.4%) as a yellow solid. ¹H NMR δ 1.22 (2H, q), 1.55 (1H, d), 1.67 (1H, s), 1.73-1.99 (5H, m), 2-2.09 (1H, m), 2.23 (2H, d), 2.50 (1H, s), 2.53-2.58 (4H, m), 2.65 (5H, dd), 2.84-3.05 (2H, m), 3.2-3.33 (5H, m), 3.37 (1H, s), 3.62 (2H, d), 5.11 (1H, dd), 6.86 (3H, dd), 7.15 (3H, dd), 7.45 (1H, d), 7.73 (1H, d), 8.32 (1H, s), 11.12 (1H, s), 12.29 (1H, s); m/z: ES⁺ [M+H]⁺=791.35.

### Example 55

### Intermediate 55a

### 2-(2,6-Dioxopiperidin-3-yl)-5-fluoro-1H-isoindole-1,3(2H)-dione

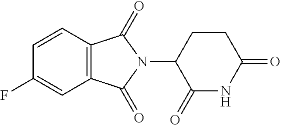 [SMILES: O=C1CCC(N2C(=O)c3ccc(F)cc3C2=O)C(=O)N1]

Sodium acetate (14.82 g, 180.61 mmol) was added to 3-aminopiperidine-2,6-dione hydrochloride (14.86 g, 90.30 mmol) and 5-fluoro-2-benzofuran-1,3-dione (15 g, 90.30 mmol) in AcOH (200 mL). The resulting mixture was stirred at 120° C. for 16 h. The solvent was evaporated and the residue was poured into water (300 mL) which was stirred for 10 mins. The solid was filtered under vacuum and was washed with water (100 mL) to give the title compound (22.0 g, 88%) as a grey solid. ¹H NMR δ 2.06 (1H, ddt, J=3.2, 5.8, 11.0 Hz), 2.50-2.65 (2H, m), 2.88 (1H, m, J=5.5, 14.0, 17.5 Hz), 5.15 (1H, dd, J=5.4, 12.9 Hz), 7.71 (1H, m, J=2.3, 8.2, 9.4 Hz), 7.83 (1H, dd, J=2.3, 7.5 Hz), 7.99 (1H, dd, J=4.5, 8.3 Hz), 11.11 (1H, s); m/z: ES⁺ [M+H]⁺=277.0.

### Intermediate 55b

### tert-Butyl 4-[2-(2,6-dioxopiperidin-3-yl)-1,3-dioxo-2,3-dihydro-1H-isoindol-5-yl]piperazine-1-carboxylate

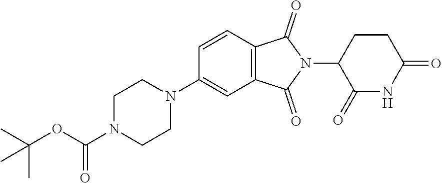 [SMILES: CC(C)(C)OC(=O)N1CCN(c2ccc3c(c2)C(=O)N(C2CCC(=O)NC2=O)C3=O)CC1]

tert-Butyl piperazine-1-carboxylate (0.742 g, 3.98 mmol), DIPEA (1.95 mL, 10.86 mmol) and 2-(2,6-Dioxopiperidin-3-yl)-5-fluoro-1H-isoindole-1,3(2H)-dione (1.0 g, 3.62 mmol) were dissolved in NMP (15 mL) and was stirred at 140° C. under microwave heating for 2 h. The mixture was cooled to RT, diluted with water (50 mL) and extracted with EtOAc (2×50 mL). The combined organics were washed with NaCl solution (2×50 mL) and dried over Na₂SO₄, filtered and evaporated to afford crude product. The crude product was purified by flash silica chromatography, elution gradient 0 to 100% EtOAc in heptane to give the title compound (2.400 g, 32.6%) as a yellow solid. ¹H NMR δ 1.43 (9H, s), 2.03 (1H, dd), 2.54-2.65 (2H, m), 2.89 (1H, m), 3.48 (8H, s), 5.08 (1H, dd), 7.25 (1H, dd), 7.35 (1H, d), 7.70 (1H, d), 11.06 (1H, s); m/z: ES⁺ [M-BOC]=343.0.

### Example 55

### 4-Chloro-7-(4-{4-[4-({4-[2-(2,6-dioxopiperidin-3-yl)-1,3-dioxo-2,3-dihydro-1H-isoindol-5-yl]piperazin-1-yl}methyl)piperidin-1-yl]phenyl}piperidin-1-yl)-1H-indole-3-carbonitrile

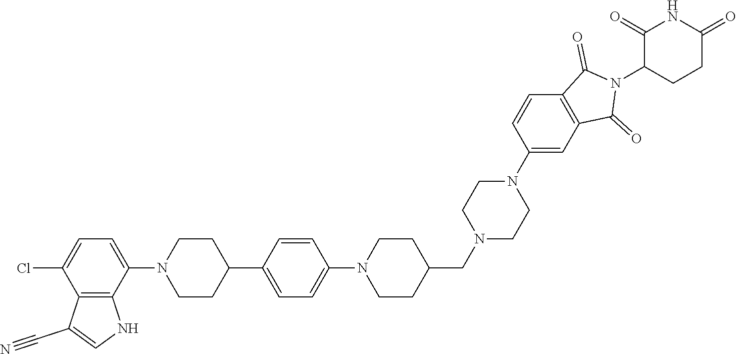 [SMILES: N#Cc1c[nH]c2c(N3CCC(c4ccc(N5CCC(CN6CCN(c7ccc8c(c7)C(=O)N(C7CCC(=O)NC7=O)C8=O)CC6)CC5)cc4)CC3)ccc(Cl)c12]

The title compound was prepared using methodology described in example 7 using intermediate 1f and tert-butyl 4-[2-(2,6-dioxopiperidin-3-yl)-1,3-dioxo-2,3-dihydro-1H-isoindol-5-yl]piperazine-1-carboxylate to give the title compound (27 mg, 28.0%) as a yellow solid. ¹H NMR δ 1.23-1.4 (3H, m), 1.68-1.81 (1H, m), 1.83-1.95 (4H, m), 1.96-2.16 (3H, m), 2.29 (2H, d), 2.62-2.72 (4H, m), 2.84 (2H, t), 2.9-3.03 (1H, m), 3.46-3.53 (6H, m), 3.71 (2H, d), 5.04-5.23 (1H, m), 6.83-7.01 (3H, m), 7.14-7.25 (3H, m), 7.29-7.36 (1H, m), 7.41 (1H, d), 7.75 (1H, d), 8.23 (1H, s), 8.38 (1H, s); m/z: ES⁺ [M+H]⁺=773.7.

### Example 56

### 7-{4-[4-(4-{4-[2-(2,6-Dioxopiperidin-3-yl)-1,3-dioxo-2,3-dihydro-1H-isoindol-5-yl]piperazin-1-yl}butoxy)phenyl]piperidin-1-yl}-4-methyl-1H-indole-3-carbonitrile

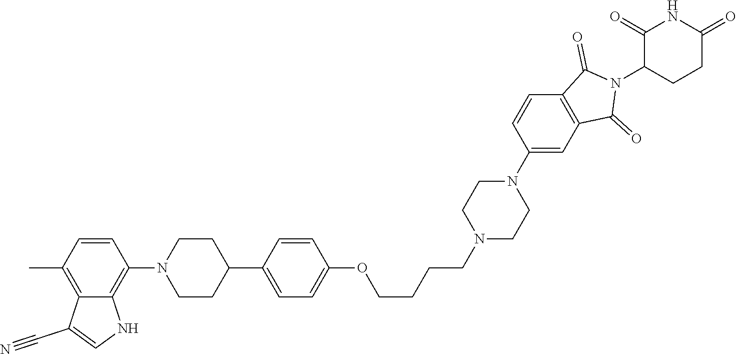 [SMILES: Cc1ccc(N2CCC(c3ccc(OCCCCN4CCN(c5ccc6c(c5)C(=O)N(C5CCC(=O)NC5=O)C6=O)CC4)cc3)CC2)c2[nH]cc(C#N)c12]

The title compound was prepared using methodology described in example 48 using intermediate 48a and intermediate 55b to give the title compound in the form of a formate salt (70 mg, 38.6%) as a yellow solid. ¹H NMR δ 1.57-1.69 (2H, m), 1.7-1.8 (2H, m), 1.85 (2H, d), 1.92-2.06 (3H, m), 2.36-2.47 (3H, m), 2.49-2.54 (4H, m), 2.57-2.63 (4H, m), 2.75 (2H, t), 2.83-2.95 (1H, m), 3.34-3.48 (7H, m), 4.00 (2H, t), 5.07 (1H, dd), 6.78 (1H, d), 6.83-6.96 (3H, m), 7.17-7.24 (2H, m), 7.24-7.29 (1H, m), 7.34 (1H, d), 7.68 (1H, d), 8.15 (1H, s), 8.16 (1H, d), 11.07 (1H, s), 11.85-11.95 (1H, m); m/z: ES⁺ [M+H]⁺=728.5.

### Example 57

### Intermediate 57a

### 7-Bromo-1H-indole-3-carbonitrile

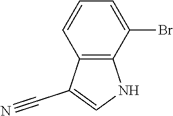 [SMILES: N#Cc1c[nH]c2c(Br)cccc12]

Sulfurisocyanatidic chloride (1.33 ml, 15.30 mmol) was added dropwise to 7-bromo-1H-indole (1.0 g, 5.10 mmol) in MeCN (8.0 mL) and DMF (2.0 mL) at 0° C. The resulting mixture was stirred at RT for 2 h. The reaction mixture was diluted with EtOAc (20 mL), washed with NaHCO₃ solution (20 mL), water (20 mL) and NaCl solution. The organic layer was dried over Na₂SO₄, filtered and evaporated to afford crude product. The crude product was purified by flash silica chromatography, elution gradient 0 to 50% EtOAc in Et₂O to give the title compound (0.900 g, 80%) as a white solid. ¹H NMR (CDCl₃) δ 7.19 (1H, t), 7.49 (1H, dd), 7.72 (1H, dt), 7.80 (1H, d), 8.94 (1H, s); m/z: ES⁺ [M+H]⁺=221.15.

### Intermediate 57b

### 7-(4-{4-[3-(1,3-Dioxolan-2-yl)propoxy]phenyl}piperidin-1-yl)-1H-indole-3-carbonitrile

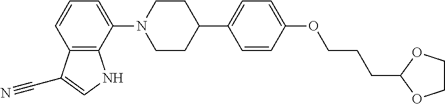 [SMILES: N#Cc1c[nH]c2c(N3CCC(c4ccc(OCCCC5OCCO5)cc4)CC3)cccc12]

Pd-PEPPSI-IHept^{C1} (30.0 mg, 0.03 mmol) was added to intermediate 32c (300 mg, 1.03 mmol), 7-bromo-1H-indole-3-carbonitrile (273 mg, 1.24 mmol) and LHMDS (1M in THF, 3.6 mL, 3.60 mmol) in THF (4 mL) under nitrogen. The resulting mixture was stirred at 60° C. for 2 h. The reaction was evaporated to dryness and purified by flash silica chromatography, elution gradient 0 to 50% EtOAc in Et₂O to give the title compound (0.266 g, 59.9%) as a yellow solid. ¹H NMR (CDCl₃) δ 1.19-1.32 (1H, m), 1.79-1.98 (3H, m), 2.00 (4H, s), 2.66 (1H, d), 2.94 (2H, s), 3.47 (2H, d), 3.80-3.91 (2H, m), 3.91-4.06 (4H, m), 4.95 (1H, t), 6.82-6.93 (2H, m), 7.00 (1H, d), 7.16-7.30 (3H, m), 7.49 (1H, d), 7.71 (1H, d), 8.81 (1H, s); m/z: ES⁺ [M+H]⁺=432.3.

### Example 57

### 7-{4-[4-(4-{4-[2-(2,6-Dioxopiperidin-3-yl)-1-oxo-2,3-dihydro-1H-isoindol-5-yl]piperazin-1-yl}butoxy)phenylpiperidin-1-yl}-1H-indole-3-carbonitrile

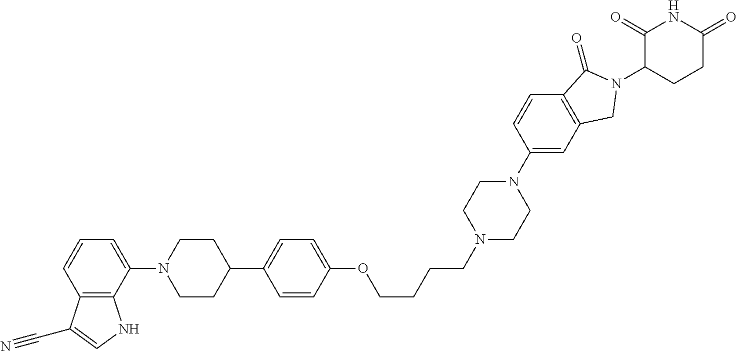 [SMILES: N#Cc1c[nH]c2c(N3CCC(c4ccc(OCCCCN5CCN(c6ccc7c(c6)CN(C6CCC(=O)NC6=O)C7=O)CC5)cc4)CC3)cccc12]

The title compound was prepared using methodology described in example 7 using 7-(4-{4-[3-(1,3-dioxolan-2-yl)propoxy]phenyl}piperidin-1-yl)-1H-indole-3-carbonitrile and intermediate 1i and purified by C18-flash chromatography, elution gradient 5 to 100% MeCN in water (1% NH₄HCO₃) to give the title compound (47 mg, 29.0%) as a white solid. ¹H NMR δ 1.23 (1H, s), 1.53-2.06 (10H, m), 2.17-2.46 (3H, m), 2.53 (2H, s), 2.56-2.70 (2H, m), 2.70-2.99 (3H, m), 3.28 (4H, t), 3.46 (2H, d), 3.99 (2H, t), 4.06-4.42 (2H, m), 4.98-5.10 (1H, m), 6.89 (3H, t), 7.05 (2H, d), 7.10-7.31 (4H, m), 7.52 (1H, d), 8.18 (1H, d), 10.93 (1H, s), 11.95 (1H, s); m/z: ES⁺ [M+H]⁺=700.4.

### Example 58

### 7-{4-[4-(4-{4-[4-(2,4-Dioxo-1,3-diazinan-1-yl)-1H-indol-1-yl]piperidin-1-yl}butoxy)phenyl]piperidin-1-yl}-1H-indole-3-carbonitrile

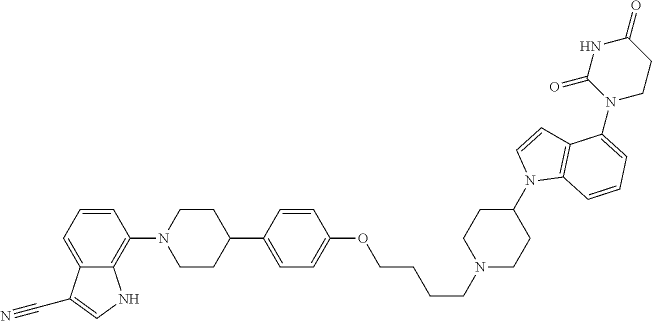 [SMILES: N#Cc1c[nH]c2c(N3CCC(c4ccc(OCCCCN5CCC(n6ccc7c(N8CCC(=O)NC8=O)cccc76)CC5)cc4)CC3)cccc12]

The title compound was prepared using methodology described in example 23 using intermediate 57b and intermediate 5f and purified by C18-flash chromatography, elution gradient 5 to 100% MeCN in water (1% NH₄HCO₃) to give the title compound (25 mg, 56.8%) as a white solid. ¹H NMR δ 1.24-1.36 (1H, m), 1.72-1.87 (4H, m), 1.89-1.95 (2H, m), 1.98-2.06 (2H, m), 2.09-2.17 (4H, m), 2.63-2.72 (1H, m), 2.76-2.93 (5H, m), 3.53 (2H, d), 3.84 (2H, t), 4.08 (2H, t), 4.46-4.73 (1H, m), 6.50 (1H, d), 6.9-7.07 (4H, m), 7.17-7.42 (5H, m), 7.49-7.67 (2H, m), 8.20 (1H, s), 8.25 (1H, d), 10.38 (1H, s) (4H under water); m/z: ES⁺ [M+H]⁺=684.5.

### Example 59

### Intermediate 59a

### 7-(4-{4-[4-(1,3-Dioxolan-2-yl)piperidin-1-yl]phenyl}piperidin-1-yl)-1H-indole-3-carbonitrile

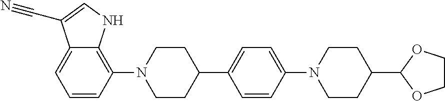 [SMILES: N#Cc1c[nH]c2c(N3CCC(c4ccc(N5CCC(C6OCCO6)CC5)cc4)CC3)cccc12]

The title compound was prepared using methodology described in intermediate 11c using intermediate 57a and intermediate 1e to give the title compound (110 mg, 25.6%) as a white solid. m/z: ES⁺ [M+H]⁺=457.3.

### Example 59

### 7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-1H-indole-3-carbonitrile

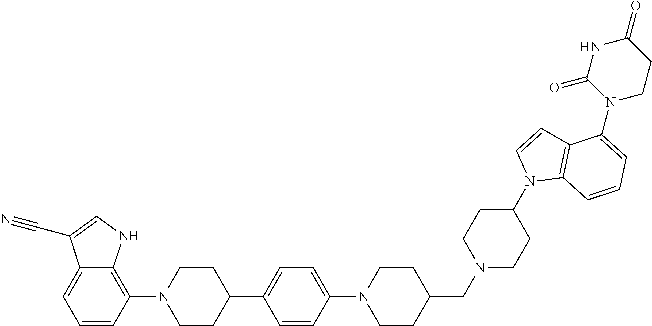 [SMILES: N#Cc1c[nH]c2c(N3CCC(c4ccc(N5CCC(CN6CCC(n7ccc8c(N9CCC(=O)NC9=O)cccc87)CC6)CC5)cc4)CC3)cccc12]

The title compound was prepared using methodology described in example 23 using 7-(4-{4-[4-(1,3-dioxolan-2-yl)piperidin-1-yl]phenyl}piperidin-1-yl)-1H-indole-3-carbonitrile and intermediate 5f and purified by C18-flash chromatography, elution gradient 5 to 100% MeCN in water (1% NH₄HCO₃) to give the title compound (55 mg, 35.4%) as a white solid. ¹H NMR δ 1.24 (2H, q), 1.67 (1H, s), 1.84 (4H, d), 1.91-2.07 (6H, m), 2.18 (2H, t), 2.25 (2H, d), 2.53-2.68 (3H, m), 2.72-2.83 (4H, m), 3.00 (2H, d), 3.46 (2H, d), 3.65 (2H, d), 3.78 (2H, t), 4.28-4.45 (1H, m), 6.42 (1H, d), 6.85-6.99 (4H, m), 7.1-7.2 (4H, m), 7.27 (1H, d), 7.48-7.57 (2H, m), 8.18 (1H, s), 10.33 (1H, s), 11.93 (1H, s); m/z: ES⁺ [M+H]⁺=709.4.

### Example 60

### Intermediate 60a

### Benzyl 4-(5-bromopyridin-2-yl)piperidine-1-carboxylate

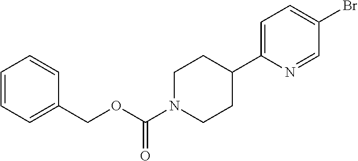 [SMILES: O=C(OCc1ccccc1)N1CCC(c2ccc(Br)cn2)CC1]

Benzyl carbonchloridate (1.1 g, 6.47 mmol) was added to 5-bromo-2-(piperidin-4-yl)pyridine (1.3 g, 5.39 mmol) and TEA (1.88 mL, 13.48 mmol) in THF (30 mL) at RT for 16 h. The mixture was then poured into water (50 mL), extracted with EtOAc (50 mL), dried over Na₂SO₄, filtered and evaporated to afford crude product. The crude product was purified by flash silica chromatography, elution gradient 0 to 30% EtOAc in Et₂O to give the title compound (1.5 g, 74.1%) as a colourless gum. ¹H NMR δ 1.46-1.66 (2H, m), 1.77-1.87 (2H, m), 2.82-3.02 (3H, m), 3.95-4.16 (2H, m), 5.08 (2H, s), 7.25-7.43 (6H, m), 7.91-8 (1H, m), 8.60 (1H, d); m/z: ES⁺ [M+H]⁺=375.1.

### Intermediate 60b

### Benzyl 4-{5-[4-(dibutoxymethyl)piperidin-1-yl]pyridin-2-yl}piperidine-1-carboxylate

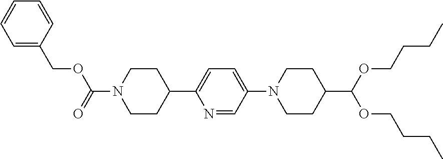 [SMILES: CCCCOC(OCCCC)C1CCN(c2ccc(C3CCN(C(=O)OCc4ccccc4)CC3)nc2)CC1]

Benzyl 4-(5-bromopyridin-2-yl)piperidine-1-carboxylate (1.45 g, 3.86 mmol) was added to 4-(dibutoxymethyl)piperidine (1.13 g, 4.64 mmol), Cs₂CO₃ (2.52 g, 7.73 mmol), Bis(tri-t-butylphosphine)palladium(0) (0.197 g, 0.39 mmol) and tri-tert-butylphosphonium tetrafluoroborate (0.112 g, 0.39 mmol) in 1,4-dioxane (1 mL) under nitrogen. The resulting mixture was stirred at 100° C. for 16 h. The reaction was then cooled to RT and evaporated to dryness to afford crude product. The crude product was purified by flash silica chromatography, elution gradient 0 to 40% EtOAc in Et₂O to give the title compound (0.85 g, 40.9%) as a yellow gum. No nmr data m/z: ES⁺ [M+H]⁺=538.5.

### Intermediate 60c

### 5-[4-Dibutoxymethyl)piperidin-1-yl]-2-(piperidin-4-yl)pyridine

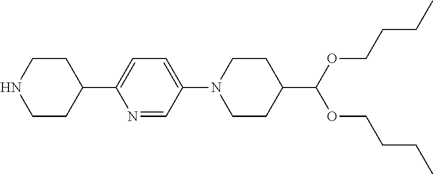 [SMILES: CCCCOC(OCCCC)C1CCN(c2ccc(C3CCNCC3)nc2)CC1]

Pd/C (10%, 0.317 g, 0.30 mmol) was added to benzyl 4-{5-[4-(dibutoxymethyl)piperidin-1-yl]pyridin-2-yl}piperidine-1-carboxylate (0.80 g, 1.49 mmol) in MeOH (15 mL) at RT under an atmosphere of hydrogen for 2 h. The mixture was filtered through Celite® and evaporated to dryness to give the title compound (0.55 g, 92%) as a yellow gum. ¹H NMR δ 0.89 (6H, t), 1.3-1.38 (5H, m), 1.46-1.53 (5H, m), 1.65-1.77 (6H, m), 2.59 (4H, t), 2.99 (2H, d), 3.17 (2H, d), 3.39 (2H, dt), 3.56 (2H, dt), 3.68 (2H, d), 4.09 (1H, d), 4.20 (1H, d), 7.04 (1H, d), 7.24 (1H, dd), 8.17 (1H, d); m/z: ES⁺ [M+H]⁺=404.4.

### Intermediate 60d: 4-Chloro-7-(4-{5-[4-(dibutoxymethyl)piperidin-1-yl]pyridin-2-yl}piperidin-1-yl)-1H-indole-3-carbonitrile

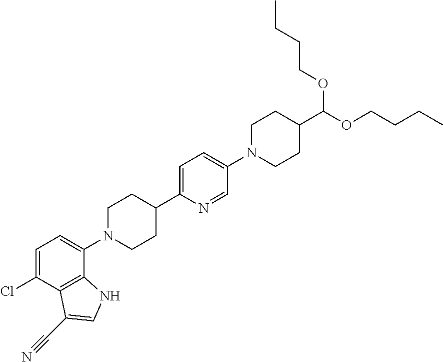 [SMILES: CCCCOC(OCCCC)C1CCN(c2ccc(C3CCN(c4ccc(Cl)c5c(C#N)c[nH]c45)CC3)nc2)CC1]

Pd-PEPPSI-IHept^{C1} (60.3 mg, 0.06 mmol) was added to 5-[4-dibutoxymethyl)piperidin-1-yl]-2-(piperidin-4-yl)pyridine (500 mg, 1.24 mmol), intermediate 1b (380 mg, 1.49 mmol) and LHMDS (1M in THF, 4.34 mL, 4.34 mmol) in THF (8 mL) under nitrogen. The resulting mixture was stirred at 60° C. for 3 h. The reaction was cooled to RT and quenched with MeOH. The solvent was removed under reduced pressure to afford crude product. The crude product was purified by flash silica chromatography, elution gradient 0 to 50% EtOAc in Et₂O to give the title compound (450 mg, 62.8%) as a yellow solid. ¹H NMR δ 0.87 (6H, t), 1.24-1.41 (6H, m), 1.41-1.55 (4H, m), 1.63-1.78 (3H, m), 1.82-1.92 (2H, m), 1.94-2.15 (2H, m), 2.53-2.67 (2H, m), 2.68-2.83 (3H, m), 3.33-3.44 (4H, m), 3.48-3.61 (2H, m), 3.69 (2H, d), 4.18 (1H, d), 6.83 (1H, d), 7.12 (2H, dd), 7.26 (1H, dd), 8.21 (1H, d), 8.29 (1H, d), 12.28 (1H, s); m/z: ES⁺ [M+H]⁺=578.3.

### Example 60: 4-Chloro-7-(4-{5-[4-({4-[2-(2,6-dioxopiperidin-3-yl)-7-methoxy-1-oxo-2,3-dihydro-1H-isoindol-5-yl]piperazin-1-yl}methyl)piperidin-1-yl]pyridin-2-yl}piperidin-1-yl)-1H-indole-3-carbonitrile

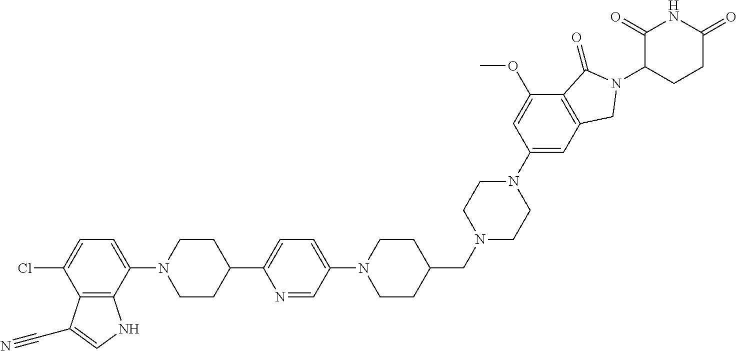 [SMILES: COc1cc(N2CCN(CC3CCN(c4ccc(C5CCN(c6ccc(Cl)c7c(C#N)c[nH]c67)CC5)nc4)CC3)CC2)cc2c1C(=O)N(C1CCC(=O)NC1=O)C2]

The title compound was prepared using methodology described in example 24 using 4-chloro-7-(4-{5-[4-(dibutoxymethyl)piperidin-1-yl]pyridin-2-yl}piperidin-1-yl)-1H-indole-3-carbonitrile and intermediate 7c to give the title compound (41 mg, 30.0%) as a white solid. ¹H NMR δ 1.23 (2H, q), 1.71 (1H, s), 1.83 (2H, d), 1.85-1.95 (3H, m), 2-2.15 (2H, m), 2.23 (2H, d), 2.25-2.39 (1H, m), 2.49 (2H, s), 2.51 (1H, s), 2.54 (1H, d), 2.58 (1H, d), 2.63-2.73 (3H, m), 2.77 (2H, t), 2.83-2.96 (1H, m), 3.30 (4H, s), 3.41 (2H, d), 3.70 (2H, d), 3.83 (3H, s), 4.10 (1H, d), 4.23 (1H, d), 4.97 (1H, dd), 6.49 (1H, d), 6.61 (1H, s), 6.85 (1H, d), 7.14 (2H, dd), 7.29 (1H, dd), 8.24 (1H, d), 8.31 (1H, s), 10.92 (1H, s), 12.30 (1H, s); m/z: ES⁺ [M+H]⁺=790.3.

### Example 61

### 4-Chloro-7-(4-{5-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]pyridin-2-yl}piperidin-1-yl)-1H-indole-3-carbonitrile

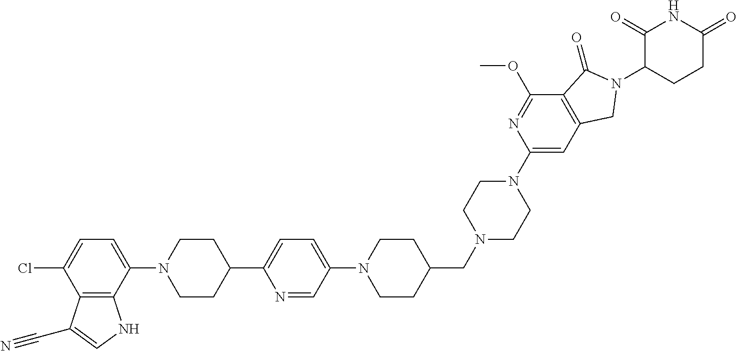 [SMILES: COc1nc(N2CCN(CC3CCN(c4ccc(C5CCN(c6ccc(Cl)c7c(C#N)c[nH]c67)CC5)nc4)CC3)CC2)cc2c1C(=O)N(C1CCC(=O)NC1=O)C2]

The title compound was prepared using methodology described in example 24 using intermediate 60d and intermediate 30e to give the title compound (40 mg, 29.2%) as a white solid. ¹H NMR δ 1.23 (2H, q), 1.71 (1H, s), 1.81 (2H, d), 1.85-1.94 (3H, m), 2.07 (2H, q), 2.14-2.24 (2H, m), 2.24-2.37 (1H, m), 2.46 (3H, s), 2.52-2.6 (1H, m), 2.62-2.73 (3H, m), 2.72-2.82 (3H, m), 2.82-2.96 (1H, m), 3.41 (2H, d), 3.51-3.77 (6H, m), 3.89 (3H, s), 4.13 (1H, d), 4.27 (1H, d), 4.97 (1H, dd), 6.48 (1H, s), 6.85 (1H, d), 7.14 (2H, dd), 7.29 (1H, dd), 8.22-8.34 (2H, m), 10.93 (1H, s), 12.30 (1H, s); m/z: ES⁺ [M+H]⁺=791.3.

### Example 62: 4-Chloro-7-(4-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl)-1H-indole-3-carbonitrile

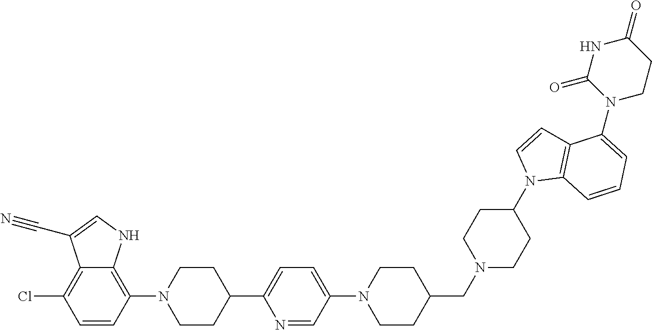 [SMILES: N#Cc1c[nH]c2c(N3CCC(c4ccc(N5CCC(CN6CCC(n7ccc8c(N9CCC(=O)NC9=O)cccc87)CC6)CC5)cn4)CC3)ccc(Cl)c12]

Intermediate 60d (100 mg, 0.17 mmol) was stirred in formic acid (1 mL) at 60° C. for 1 h, cooled to RT and evaporated to dryness. The residue was added to a mixture of sodium acetate (42.6 mg, 0.52 mmol) and intermediate 5f (96 mg, 0.21 mmol) in NMP (2 mL). The resulting mixture was stirred at RT for 16 h. The reaction mixture was quenched with NaHCO₃ solution (10 mL) and purified by flash C18-flash chromatography, elution gradient 0 to 70% MeCN in water (0.1% NH₄HCO₃) to give the title compound (36.0 mg, 28.0%) as a yellow solid. ¹H NMR δ 1.26 (2H, d), 1.73 (1H, s), 1.81-2.16 (10H, m), 2.28 (3H, s), 2.65-2.84 (7H, m), 3.04 (2H, s), 3.42 (2H, d), 3.72 (2H, d), 3.79 (2H, t), 4.42 (1H, s), 6.43 (1H, d), 6.87 (1H, d), 6.97 (1H, d), 7.16 (4H, t), 7.31 (1H, dd), 7.5-7.57 (2H, m), 8.22-8.35 (2H, m), 10.35 (1H, s), 12.30 (1H, s); m/z: ES⁺ [M+H]⁺=744.3.

### Example 63

### Intermediate 63a

### 3-Bromo-6-[4-(dibutoxymethyl)piperidin-1-yl]pyridazine

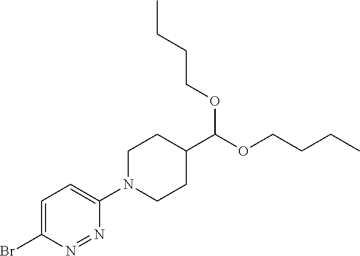 [SMILES: CCCCOC(OCCCC)C1CCN(c2ccc(Br)nn2)CC1]

DIPEA (2.71 mL, 15.51 mmol) was added to 4-(dibutoxymethyl)piperidine (1.258 g, 5.17 mmol) and 3-bromo-6-chloropyridazine (1.0 g, 5.17 mmol) in DMSO (20 mL) at RT and was stirred at 100° C. for 3 h. The reaction mixture was cooled to RT, diluted with EtOAc (100 mL), washed with saturated NH₄Cl solution (20 mL), water (25 mL), NaCl solution (25 mL), dried over Na₂SO₄ and filtered under gravity. The solvent was evaporated to afford crude product. The crude product was purified by flash silica chromatography, elution gradient 0 to 30% EtOAc in Et₂O to give the title compound (1.8 g, 87%) as a colourless gum. ¹H NMR (CDCl₃) δ 0.94 (6H, t), 1.33-1.46 (6H, m), 1.53-1.62 (4H, m), 1.84-1.98 (3H, m), 2.94 (2H, tt), 3.45 (2H, dt), 3.64 (2H, dt), 4.17 (1H, dd), 4.39 (2H, d), 6.89 (1H, dd), 7.12-7.29 (1H, m); m/z: ES⁺ [M+H]⁺=400.0.

### Intermediate 63b

### Benzyl 4-{6-[4-(dibutoxymethyl)piperidin-1-yl]pyridazin-3-yl}-3,6-dihydropyridine-1(2H)-carboxylate

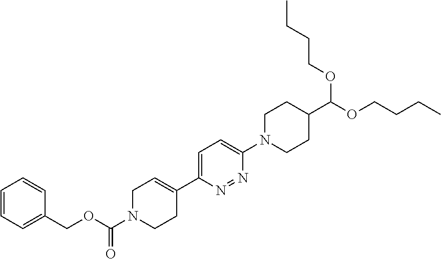 [SMILES: CCCCOC(OCCCC)C1CCN(c2ccc(C3=CCN(C(=O)OCc4ccccc4)CC3)nn2)CC1]

PdCl₂(dtbpf) (0.277 g, 0.42 mmol) was added to 3-bromo-6-[4-(dibutoxymethyl)piperidin-1-yl]pyridazine (1.7 g, 4.25 mmol), benzyl 4-(4,4,5,5-tetramethyl-1,3,2-dioxaborolan-2-yl)-3,6-dihydropyridine-1(2H)-carboxylate (1.60 g, 4.67 mmol) and K₂CO₃ (1.76 g, 12.74 mmol) in 1,4-dioxane:water (4:1, 20 mL) at RT under nitrogen. The resulting mixture was stirred at 80° C. for 1 h. The reaction was cooled to RT, filtered through Celite® and evaporated to dryness to afford crude product. The crude product was purified by flash silica chromatography, elution gradient 0 to 60% EtOAc in Et₂O to give the title compound (1.7 g, 74.6%) as a colourless gum. ¹H NMR (CDCl₃) δ 0.94 (6H, t), 1.35-1.45 (6H, m), 1.52-1.62 (4H, m), 1.92 (4H, t), 2.83 (2H, s), 2.95 (2H, t), 3.44 (2H, dt), 3.64 (2H, dt), 3.74 (2H, dd), 4.17 (1H, d), 4.22 (2H, q), 4.46 (2H, d), 5.19 (2H, s), 6.31 (1H, s), 6.94 (1H, d), 7.35-7.45 (5H, m); m/z: ES⁺ [M+H]⁺=537.1.

### Intermediate 63c

### 3-[4-(Dibutoxymethyl)piperidin-1-yl]-6-(piperidin-4-yl)pyridazine

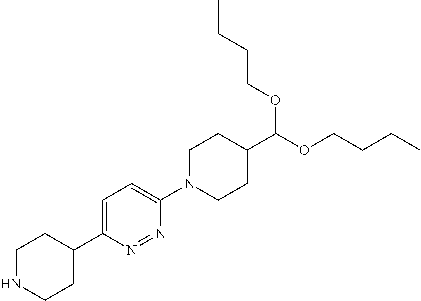 [SMILES: CCCCOC(OCCCC)C1CCN(c2ccc(C3CCNCC3)nn2)CC1]

Pd/C (10%, 1.58 g, 1.49 mmol) was added to benzyl 4-{6-[4-(dibutoxymethyl)piperidin-1-yl]pyridazin-3-yl}-3,6-dihydropyridine-1(2H)-carboxylate (1.6 g, 2.98 mmol) in MeOH (20 mL) at RT under an atmosphere of hydrogen for 3 h. The mixture was filtered through Celite® and evaporated to dryness to give the title compound (1.0 g, 83%) as a yellow gum. ¹H NMR (CDCl₃) δ 0.92 (6H, t), 1.31-1.43 (6H, m), 1.49-1.61 (4H, m), 1.71-2 (7H, m), 2.73-3 (5H, m), 3.27 (2H, d), 3.37-3.45 (2H, m), 3.62 (2H, dt), 4.16 (1H, d), 4.36 (2H, d), 6.91 (1H, d), 7.12 (1H, d) NH not observed; m/z: ES⁺ [M+H]⁺=405.2.

### Intermediate 63d

### 4-Chloro-7-(4-{6-[4-(dibutoxymethyl)piperidin-1-yl]pyridazin-3-yl}piperidin-1-yl)-1H-indole-3-carbonitrile

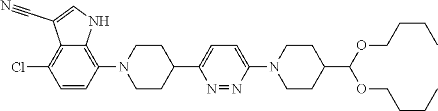 [SMILES: CCCCOC(OCCCC)C1CCN(c2ccc(C3CCN(c4ccc(Cl)c5c(C#N)c[nH]c45)CC3)nn2)CC1]

LHMDS (1M in THF, 6.29 mL, 6.29 mmol) was added to 3-[4-(dibutoxymethyl)piperidin-1-yl]-6-(piperidin-4-yl)pyridazine (800 mg, 1.98 mmol), intermediate 1b (459 mg, 1.80 mmol), CPhos (78 mg, 0.18 mmol) and [Pd(cinnamyl)Cl]₂ (93 mg, 0.18 mmol) in THF (7 mL) under nitrogen. The resulting mixture was stirred at 60° C. for 2 h. The reaction mixture was cooled to RT, diluted with EtOAc (100 mL), washed with saturated NH₄Cl solution (50 mL), water (50 mL), NaCl solution (50 mL) and dried over Na₂SO₄. The solvent was evaporated to afford crude product. The crude product was purified by flash silica chromatography, elution gradient 0 to 80% EtOAc in Et₂O to give the title compound (500 mg, 48.0%) as a yellow solid. ¹H NMR (CDCl₃) δ 0.94 (6H, t), 1.38-1.43 (4H, m), 1.54-1.62 (4H, m), 1.89 (3H, d), 1.99 (2H, d), 2.21-2.43 (3H, m), 2.63 (1H, d), 2.87 (5H, dt), 3.37-3.56 (4H, m), 3.64 (2H, dt), 4.19 (1H, d), 4.35 (2H, t), 6.82 (1H, d), 6.96 (1H, d), 7.14 (2H, dd), 7.71 (1H, d), 11.09 (1H, s); m/z: ES⁺ [M+H]⁺=579.0.

### Example 63

### 4-Chloro-7-(4-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl)-1H-indole-3-carbonitrile

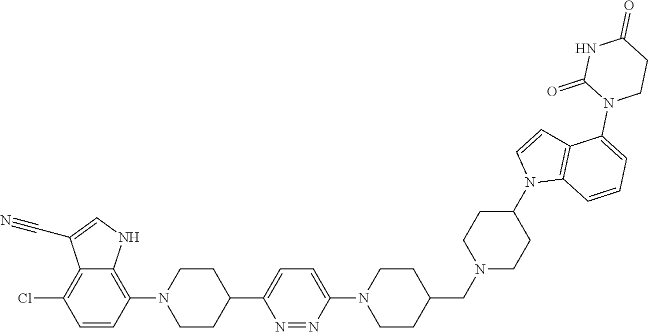 [SMILES: N#Cc1c[nH]c2c(N3CCC(c4ccc(N5CCC(CN6CCC(n7ccc8c(N9CCC(=O)NC9=O)cccc87)CC6)CC5)nn4)CC3)ccc(Cl)c12]

4-Chloro-7-(4-{6-[4-(dibutoxymethyl)piperidin-1-yl]pyridazin-3-yl}piperidin-1-yl)-1H-indole-3-carbonitrile (120 mg, 0.21 mmol) was added to formic acid (2 mL). The resulting mixture was stirred at 40° C. for 1 h and then evaporated to dryness. Intermediate 5f (100 mg, 0.21 mmol) and sodium acetate (51.0 mg, 0.62 mmol) were added to the residue in NMP (2 mL) which was stirred at 40° C. for 16 h. The reaction mixture was purified by flash C18-flash chromatography, elution gradient 0 to 30% MeCN in water (0.1% formic acid). The MeCN was removed under reduced pressure and the aqueous solution was basified with NaHCO₃ solution. The mixture was filtered through Celite® and the solvent was evaporated to give the title compound (65.0 mg, 42.1%) as a white solid. ¹H NMR δ 1.15 (2H, d), 1.84 (3H, d), 1.9-2 (4H, m), 2-2.31 (8H, m), 2.69-2.93 (7H, m), 3.02 (2H, s), 3.44 (2H, d), 3.78 (2H, t), 4.34 (3H, d), 6.42 (1H, d), 6.87 (1H, d), 6.96 (1H, d), 7.07-7.2 (2H, m), 7.26 (1H, d), 7.33 (1H, d), 7.45-7.66 (2H, m), 8.32 (1H, d), 10.33 (1H, s), 12.30 (1H, s); m/z: ES⁺ [M+H]⁺=745.4.

### Example 64

### 4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridazin-3-yl}piperidin-1-yl)-1H-indole-3-carbonitrile

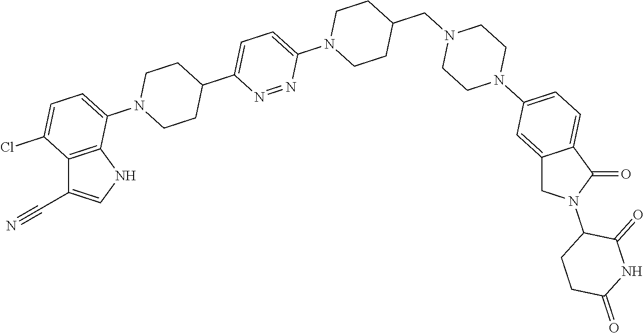 [SMILES: N#Cc1c[nH]c2c(N3CCC(c4ccc(N5CCC(CN6CCN(c7ccc8c(c7)CN(C7CCC(=O)NC7=O)C8=O)CC6)CC5)nn4)CC3)ccc(Cl)c12]

The title compound was prepared using methodology described in example 63 using intermediate 63d and intermediate 1j to give the title compound (55 mg, 21%) as a white solid. ¹H NMR δ 1.06-1.27 (2H, m), 1.75-1.9 (3H, m), 1.9-2.01 (3H, m), 2.01-2.14 (2H, m), 2.21 (2H, d), 2.29-2.46 (2H, m), 2.54-2.66 (2H, m), 2.72-2.99 (6H, m), 3.26-3.37 (6H, m), 3.59 (2H, d), 4.11-4.43 (4H, m), 5.05 (1H, dd), 6.76 (1H, d), 7.08 (3H, d), 7.18-7.39 (2H, m), 7.52 (1H, d), 8.21 (1H, s), 10.95 (1H, s), 12.25 (1H, s); m/z: ES⁺ [M+H]⁺=761.3.

### Example 65

### Intermediate 65a

### tert-Butyl 5-chloro-5′,6′-dihydro[2,3′-bipyridine]-1′(4′H)-carboxylate

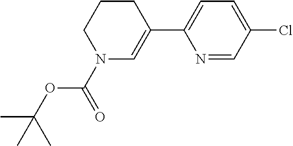 [SMILES: CC(C)(C)OC(=O)N1C=C(c2ccc(Cl)cn2)CCC1]

The title compound was prepared using methodology described in intermediate 63b using 2-bromo-5-chloropyridine and tert-butyl 5-(4,4,5,5-tetramethyl-1,3,2-dioxaborolan-2-yl)-3,4-dihydropyridine-1(2H)-carboxylate to give the title compound (1.9 g, 99%) as a brown solid. ¹H NMR δ 1.43 (9H, s), 2.30 (2H, tq), 3.47 (2H, t), 4.31 (2H, q), 6.83 (1H, dp), 7.64-7.76 (1H, m), 7.90 (1H, dd), 8.58 (1H, dd); m/z: ES⁺ [M+H]⁺=295.0.

### Intermediate 65b

### 5-Chloro-1′,4′,5′,6′-tetrahydro-2,3′-bipyridine

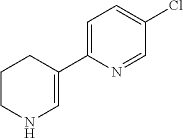 [SMILES: Clc1ccc(C2=CNCCC2)nc1]

tert-Butyl 5-chloro-5′,6′-dihydro[2,3′-bipyridine]-1′(4′H)-carboxylate (1.9 g, 6.45 mmol) was added to HCl (4M in 1,4-dioxane, 32 mL, 128.91 mmol) at RT under nitrogen and stirred for 16 h. The reaction was filtered through Celite® and evaporated to dryness to afford crude product. The crude product was purified by flash silica chromatography, elution gradient 0 to 60% EtOAc in Et₂O to give the title compound (1.2 g, 96%) as a brown solid. ¹H NMR δ 1.09-1.29 (1H, m), 2.34 (2H, qd), 3.42-3.61 (2H, m), 4.39 (2H, s), 6.84 (1H, td), 7.71 (1H, d), 7.89 (1H, dd), 8.57 (1H, s); m/z: ES⁺ [M+H]⁺=195.0.

### Intermediate 65c

### Benzyl 5-chloro-5′,6′-dihydro[2,3′-bipyridine]-1′(4′H)-carboxylate

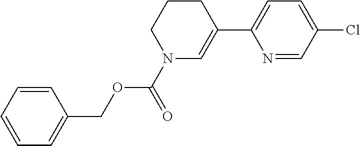 [SMILES: O=C(OCc1ccccc1)N1C=C(c2ccc(Cl)cn2)CCC1]

The title compound was prepared using methodology described in intermediate 60a using 5-chloro-1′,4′,5′,6′-tetrahydro-2,3′-bipyridine to give the title compound (1.9 g, 99%) as a brown solid. ¹H NMR δ 1.20 (1H, s), 1.49 (1H, d), 2.50 (1H, p), 3.17 (3H, dt), 3.97 (3H, dq), 6.92 (1H, td), 7.78 (1H, d), 7.94 (1H, dd), 8.57 (1H, d), 9.86 (3H, s), 11.50 (1H, s); m/z: ES⁺ [M+H]⁺=329.0.

### Intermediate 65d

### Benzyl 5-[4-(dibutoxymethyl)piperidin-1-yl]-5′,6′-dihydro[2,3′-bipyridine]-1′(4′H)-carboxylate

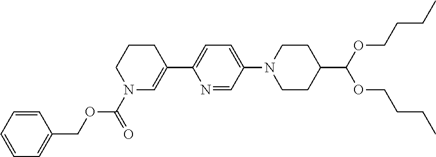 [SMILES: CCCCOC(OCCCC)C1CCN(c2ccc(C3=CN(C(=O)OCc4ccccc4)CCC3)nc2)CC1]

The title compound was prepared using methodology described in intermediate 11a using benzyl 5-chloro-5′,6′-dihydro[2,3′-bipyridine]-1′(4′H)-carboxylate to give the title compound (1.0 g, 47%) as a brown solid. ¹H NMR δ 0.88 (6H, td), 1.19-1.41 (7H, m), 1.41-1.57 (5H, m), 1.73 (3H, d), 2.29 (2H, s), 2.6-2.72 (2H, m), 3.39 (3H, dt), 3.56 (4H, dt), 3.75-3.83 (1H, m), 4.19 (1H, d), 4.38 (1H, s), 5.13 (2H, s), 6.55 (1H, d), 7.22-7.44 (6H, m), 8.21 (1H, s); m/z: ES⁺ [M+H]⁺=536.4.

### Intermediate 65e

### 5-[4-(Dibutoxymethyl)piperidin-1-yl]-2-(piperidin-3-yl)pyridine

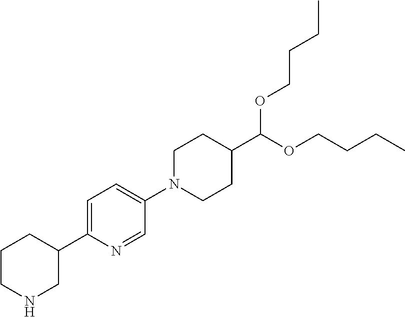 [SMILES: CCCCOC(OCCCC)C1CCN(c2ccc(C3CCCNC3)nc2)CC1]

The title compound was prepared using methodology described in intermediate 11b using benzyl 5-[4-(dibutoxymethyl)piperidin-1-yl]-5′,6′-dihydro[2,3′-bipyridine]-1′(4′H)-carboxylate to give the title compound (610 mg, 67%) as a yellow solid. ¹H NMR δ 0.88 (6H, t), 1.28-1.41 (6H, m), 1.48 (6H, dq), 1.54-1.67 (3H, m), 1.66-1.75 (3H, m), 1.87 (1H, d), 2.54-2.66 (3H, m), 2.94 (1H, s), 3.02 (1H, d), 3.37-3.42 (3H, m), 3.55 (2H, dt), 3.68 (2H, d), 4.19 (1H, d), 7.05 (1H, d), 7.18-7.32 (1H, m), 8.17 (1H, d); m/z: ES⁺ [M+H]⁺=404.4.

### Intermediates 65f and 66a

The title compounds were prepared using methodology described in intermediate 11c using 5-[4-(dibutoxymethyl)piperidin-1-yl]-2-(piperidin-3-yl)pyridine and intermediate 1b to give a racemic mixture. The enantiomers were separated by preparative chiral-HPLC on a Column: CHIRALPAK IF-3, 4.6*50 mm, 3 um; Flow rate: 1 mL/min; Gradient: 0% B; Mobile Phase A: Hex (0.5% 2M NH3-MeOH), Mobile Phase B: EtOH; Flow rate: 20 mL/min; Gradient: 25% B to give, in order of elution, intermediate 65f (isomer 1, 66 mg, 8%) and intermediate 66a (isomer 2, 88 mg, 10%) as yellow gums.

### Intermediate 65f

### 4-Chloro-7-[(3R*)-3-{5-[4-(dibutoxymethyl)piperidin-1-yl]pyridin-2-yl}piperidin-1-yl]-1H-indole-3-carbonitrile [Absolute Stereochemistry not Yet Confirmed]

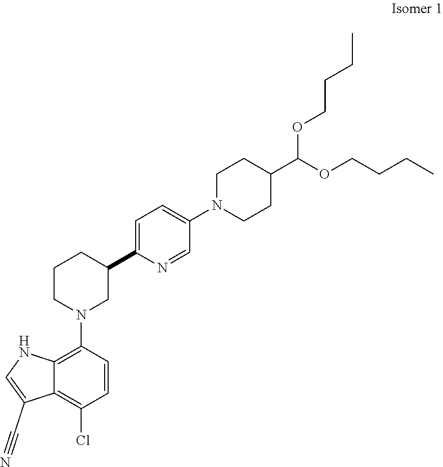

¹H NMR δ 0.88 (6H, t), 1.19-1.42 (6H, m), 1.48 (4H, dq), 1.64-1.79 (4H, m), 1.85 (2H, s), 1.98 (1H, d), 2.63 (2H, t), 2.81 (1H, d), 3.22 (4H, d), 3.39 (2H, dt), 3.56 (2H, dt), 3.74 (2H, d), 4.19 (1H, d), 6.80 (1H, d), 7.13 (1H, d), 7.22-7.29 (1H, m), 7.32 (1H, dd), 8.28 (1H, d), 8.41 (1H, d), 12.94 (1H, s); m/z: ES⁺ [M+H]⁺=578.2.

### Intermediate 66a

### 4-Chloro-7-[(3S*)-3-{5-[4-(dibutoxymethyl)piperidin-1-yl]pyridin-2-yl}piperidin-1-yl]-1H-indole-3-carbonitrile [Absolute Stereochemistry not Yet Confirmed]

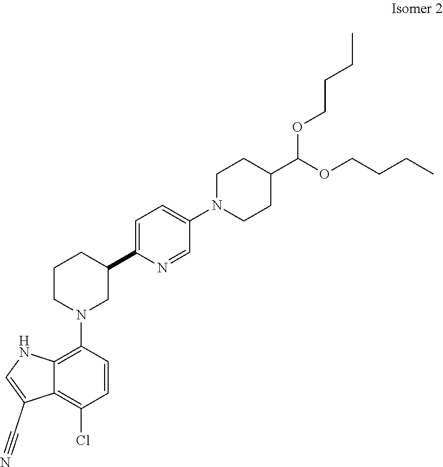

¹H NMR δ 0.88 (6H, t), 1.19-1.4 (6H, m), 1.48 (4H, dq), 1.73 (4H, d), 1.85 (2H, s), 1.98 (1H, d), 2.62 (2H, t), 2.81 (1H, d), 3.11-3.28 (4H, m), 3.39 (2H, dt), 3.55 (2H, dt), 3.73 (2H, d), 4.19 (1H, d), 6.80 (1H, d), 7.13 (1H, d), 7.25 (1H, d), 7.32 (1H, dd), 8.28 (1H, d), 8.41 (1H, d), 12.93 (1H, s); m/z: ES⁺ [M+H]⁺=578.3.

### Example 65

### 4-Chloro-7-[(3R*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile [Absolute Stereochemistry not Yet Confirmed]

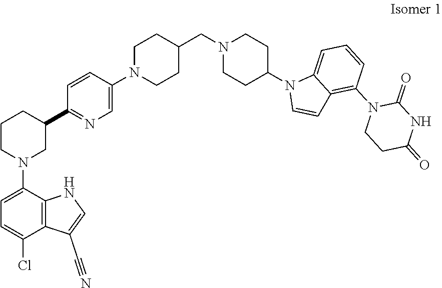

The title compound was prepared using methodology described in example 23 using intermediate 65f and intermediate 5f to give the title compound in the form of a formate salt (36 mg, 42%) as a white solid. ¹H NMR δ 1.18-1.28 (2H, m), 1.72 (2H, s), 1.8-1.89 (4H, m), 1.91-2.02 (5H, m), 2.17 (2H, t), 2.25 (2H, d), 2.63-2.83 (5H, m), 3.00 (2H, d), 3.09-3.3 (4H, m), 3.76 (4H, dt), 4.38 (1H, dt), 6.42 (1H, d), 6.79 (1H, d), 6.96 (1H, d), 7.09-7.17 (2H, m), 7.26 (1H, d), 7.34 (1H, dd), 7.47-7.56 (2H, m), 8.29 (1H, d), 8.39 (1H, s), 10.32 (1H, s), 12.93 (1H, s); m/z: ES⁺ [M+H]⁺=744.4.

### Example 66

### 4-Chloro-7-[(3S*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile [Absolute Stereochemistry not Yet Confirmed]

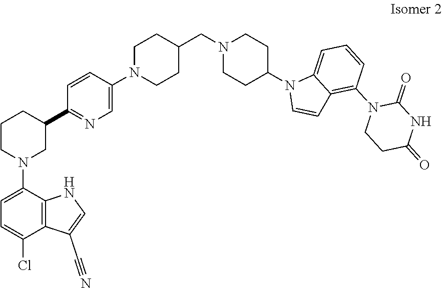

The title compound was prepared using methodology described in example 23 using intermediate 66a and intermediate 5f to give the title compound in the form of a formate salt (29 mg, 25%) as a white solid. ¹H NMR δ 1.23 (2H, q), 1.72 (2H, s), 1.84 (4H, d), 1.91-2.07 (5H, m), 2.20 (4H, dd), 2.65-2.82 (5H, m), 3.00 (2H, d), 3.11-3.3 (4H, m), 3.76 (4H, dt), 4.29-4.43 (1H, m), 6.41 (1H, d), 6.80 (1H, d), 6.96 (1H, d), 7.14 (2H, t), 7.26 (1H, d), 7.34 (1H, dd), 7.52 (2H, dd), 8.29 (1H, d), 8.41 (1H, s), 10.32 (1H, s), 12.93 (1H, s); m/z: ES⁺ [M+H]⁺=744.4.

### Example 67

### Intermediate 67a

### Benzyl 4-(5-fluoropyrazin-2-yl)-3,6-dihydropyridine-1(2H)-carboxylate

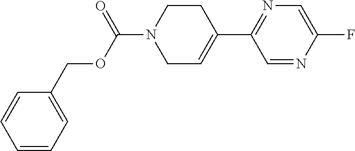 [SMILES: O=C(OCc1ccccc1)N1CC=C(c2cnc(F)cn2)CC1]

The title compound was prepared using methodology described in intermediate 63b using 2-bromo-5-fluoropyrazine and benzyl 4-(4,4,5,5-tetramethyl-1,3,2-dioxaborolan-2-yl)-3,6-dihydropyridine-1(2H)-carboxylate to give the title compound (2.6 g, 95%) as a colourless gum. ¹H NMR δ 2.59 (2H, tt), 3.64 (2H, s), 4.16 (2H, s), 5.12 (2H, s), 6.77 (1H, s), 7.27-7.42 (5H, m), 8.51 (1H, t), 8.63 (1H, dd); m/z: ES⁺ [M+H]⁺=314.1.

### Intermediate 67b

### Benzyl 4-{5-[4-(dibutoxymethyl)piperidin-1-yl]pyrazin-2-yl}-3,6-dihydropyridine-1(2H)-carboxylate

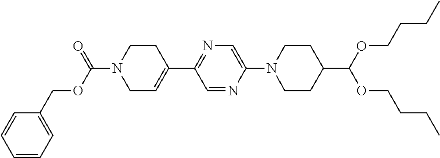 [SMILES: CCCCOC(OCCCC)C1CCN(c2cnc(C3=CCN(C(=O)OCc4ccccc4)CC3)cn2)CC1]

The title compound was prepared using methodology described in intermediate 63a using benzyl 4-(5-fluoropyrazin-2-yl)-3,6-dihydropyridine-1(2H)-carboxylate and 4-(dibutoxymethyl)piperidine to give the title compound (4.0 g, 93%) as a colourless gum. ¹H NMR δ 0.87 (6H, t), 1.08-1.29 (4H, m), 1.33 (4H, dq), 1.44-1.53 (4H, m), 1.64-1.86 (2H, m), 1.99 (1H, s), 2.51 (2H, dp), 2.82 (2H, td), 3.21-3.7 (4H, m), 3.76-4.21 (2H, m), 4.35 (3H, d), 5.11 (2H, s), 6.45 (1H, s), 7.16-7.59 (5H, m), 7.96-8.66 (2H, m); m/z: ES⁺ [M+H]⁺=537.4.

### Intermediate 67c

### 2-[4-(Dibutoxymethyl)piperidin-1-yl]-5-(piperidin-4-yl)pyrazine

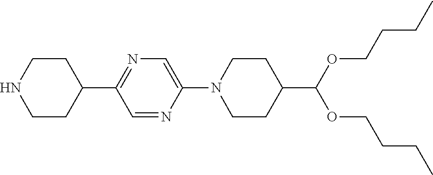 [SMILES: CCCCOC(OCCCC)C1CCN(c2cnc(C3CCNCC3)cn2)CC1]

The title compound was prepared using methodology described in intermediate 63c using benzyl 4-{5-[4-(dibutoxymethyl)piperidin-1-yl]pyrazin-2-yl}-3,6-dihydropyridine-1(2H)-carboxylate to give the title compound (3.0 g, 99%) as a colourless gum. ¹H NMR δ 0.87 (6H, td), 1.11-1.3 (2H, m), 1.31 (2H, d), 1.31-1.4 (2H, m), 1.4-1.53 (5H, m), 1.59 (2H, td), 1.67-1.82 (4H, m), 2.41-2.62 (5H, m), 2.55-3.08 (4H, m), 3.31-3.56 (4H, m), 4.17 (1H, d), 4.26 (1H, s), 7.14-8 (1H, m), 8.21 (1H, d); m/z: ES⁺ [M+H]⁺=405.4.

### Intermediate 67d

### 4-Chloro-7-(4-{5-[4-(dibutoxymethyl)piperidin-1-yl]pyrazin-2-yl}piperidin-1-yl)-1H-indole-3-carbonitrile

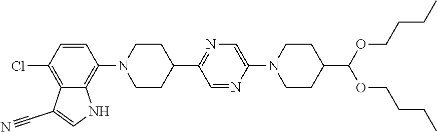 [SMILES: CCCCOC(OCCCC)C1CCN(c2cnc(C3CCN(c4ccc(Cl)c5c(C#N)c[nH]c45)CC3)cn2)CC1]

The title compound was prepared using methodology described in intermediate 11c using 2-[4-(dibutoxymethyl)piperidin-1-yl]-5-(piperidin-4-yl)pyrazine and intermediate 1b to give the title compound (0.219 g, 25%) as a white solid. ¹H NMR δ 0.88 (6H, t), 1.14-1.35 (4H, m), 1.32-1.4 (2H, m), 1.48 (4H, dq), 1.73 (2H, d), 1.76-1.92 (2H, m), 1.97-2.25 (2H, m), 2.68-2.87 (5H, m), 3.28-3.46 (5H, m), 3.55 (2H, dt), 4.18 (1H, d), 4.32 (2H, d), 6.86 (1H, d), 7.16 (1H, d), 8.03 (1H, d), 8.29 (2H, dd), 12.32 (1H, s); m/z: ES⁺ [M+H]⁺=579.3.

### Example 67

### 4-Chloro-7-(4-{5-[4-({4-[4-(2,4-dioxo-1,3-diazinan-1-yl)-1H-indol-1-yl]piperidin-1-yl}methyl)piperidin-1-ylpyrazin-2-yl}piperidin-1-yl)-1H-indole-3-carbonitrile

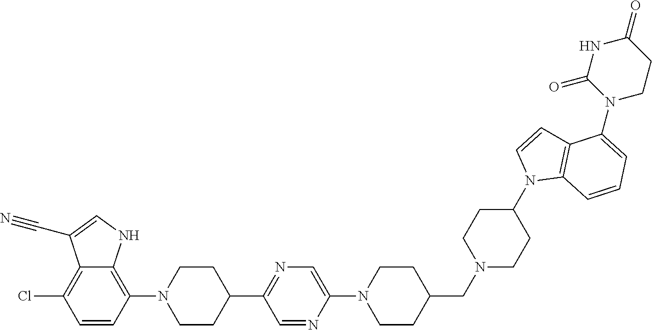 [SMILES: N#Cc1c[nH]c2c(N3CCC(c4cnc(N5CCC(CN6CCC(n7ccc8c(N9CCC(=O)NC9=O)cccc87)CC6)CC5)cn4)CC3)ccc(Cl)c12]

The title compound was prepared using methodology described in example 23 using 4-chloro-7-(4-{5-[4-(dibutoxymethyl)piperidin-1-yl]pyrazin-2-yl}piperidin-1-yl)-1H-indole-3-carbonitrile and intermediate 5f to give the title compound in the form of a formate salt (71 mg, 36.8%) as a white solid. ¹H NMR δ 1.13 (2H, d), 1.71-1.92 (8H, m), 1.99 (2H, d), 2.05-2.28 (5H, m), 2.6-2.9 (7H, m), 3.01 (2H, d), 3.42 (2H, d), 3.78 (2H, t), 4.35 (3H, dd), 6.42 (1H, d), 6.86 (1H, d), 6.96 (1H, d), 7.1-7.2 (2H, m), 7.46-7.58 (2H, m), 8.04 (1H, d), 8.26-8.36 (2H, m), 10.35 (1H, s), 12.33 (1H, s); m/z: ES⁺ [M+H]⁺=745.4.

### Example 68

### 4-Chloro-7-(4-{5-[4-({4-[2-(2,6-dioxopiperidin-3-yl)-1-oxo-2,3-dihydro-1H-isoindol-5-yl]piperazin-1-yl}methyl)piperidin-1-yl]pyrazin-2-yl}piperidin-1-yl)-1H-indole-3-carbonitrile

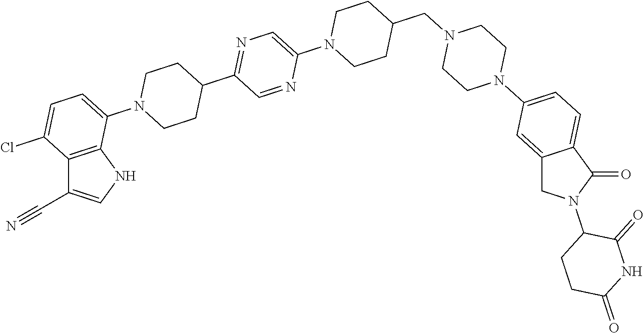 [SMILES: N#Cc1c[nH]c2c(N3CCC(c4cnc(N5CCC(CN6CCN(c7ccc8c(c7)CN(C7CCC(=O)NC7=O)C8=O)CC6)CC5)cn4)CC3)ccc(Cl)c12]

The title compound was prepared using methodology described in example 24 using intermediate 67d and intermediate 1i to give the title compound (112 mg, 56.8%) as a white solid. ¹H NMR δ 1.12 (2H, d), 1.89 (6H, m), 2.09 (2H, td), 2.29 (2H, dd), 2.49 (1H, s), 2.55-2.63 (1H, m), 2.83 (6H, m), 3.29 (4H, t), 3.40 (6H, s), 4.02-4.38 (4H, m), 5.06 (1H, dd), 6.86 (1H, d), 7.06 (2H, d), 7.17 (1H, d), 7.53 (1H, d), 8.04 (1H, d), 8.30 (2H, d), 10.97 (1H, s), 12.32 (1H, s); m/z: ES⁻ [M−H]⁻=759.3.

### Example 69

### Intermediate 69a

### 7-Ethenyl-1-methyl-1,3-dihydro-2H-benzimidazol-2-one

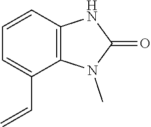

Pd(dppf)₂Cl₂-DCM (0.575 g, 0.70 mmol) was added to 7-bromo-1-methyl-1,3-dihydro-2H-benzimidazol-2-one (2.0 g, 8.81 mmol), 4,4,5,5-tetramethyl-2-vinyl-1,3,2-dioxaborolane (2.03 g, 13.21 mmol) and K₂CO₃(3.04 g, 22.02 mmol) in 1,4-dioxane:water (4:1, 48 mL) under nitrogen. The resulting mixture was stirred at 80° C. for 18 h. The reaction was cooled to RT and evaporated to dryness to afford crude product. The crude product was purified by flash silica chromatography, elution gradient 0 to 50% EtOAc in Et₂O to give the title compound (1.1 g, 71.7%) as a white solid. ¹H NMR δ 3.36 (3H, s), 5.24 (1H, dd), 5.57 (1H, dd), 6.74-6.94 (2H, m), 7.00 (1H, m), 7.26 (1H, dd), 10.80 (1H, s); m/z: ES⁺ [M+H]⁺=175.2.

### Intermediate 69b

### 3-Methyl-2-oxo-2,3-dihydro-1H-benzimidazole-4-carbaldehyde

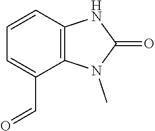

Potassium osmate dihydrate (0.212 g, 0.57 mmol) was added to 7-ethenyl-1-methyl-1,3-dihydro-2H-benzimidazol-2-one (1.0 g, 5.74 mmol), sodium meta periodate (3.68 g, 17.22 mmol) and 2,6-lutidine (1.33 ml, 11.48 mmol) in 1,4-dioxane:water (3:1, 40 mL) at RT and stirred for 2 h. The reaction mixture was quenched with water (100 mL), extracted with EtOAc (3×100 mL), dried over Na₂SO₄, filtered and evaporated to afford crude product. The crude product was purified by flash silica chromatography, elution gradient 0 to 4% MeOH in DCM to give the title compound (0.95 g, 94%) as a grey solid. ¹H NMR δ 3.58 (3H, s), 7.11 (1H, t), 7.19-7.28 (1H, m), 7.51 (1H, dd), 10.34 (1H, s), 11.30 (1H, s); m/z: ES⁺ [M+H]⁺=177.2.

### Intermediate 69c

### 1-(2,6-Dioxopiperidin-3-yl)-3-methyl-2-oxo-2,3-dihydro-1H-benzimidazole-4-carbaldehyde

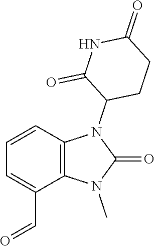 [SMILES: Cn1c(=O)n(C2CCC(=O)NC2=O)c2cccc(C=O)c21]

LHMDS (153 ml, 153.26 mmol) was added to 3-methyl-2-oxo-2,3-dihydro-1H-benzimidazole-4-carbaldehyde (9.0 g, 51.09 mmol) in THF (80 mL) at 0° C. under nitrogen. The resulting mixture was stirred at RT for 1 h. This mixture was added dropwise to a solution of 1-bromo-1,3-diazinane-2,4-dione (19.62 g, 102.17 mmol) in THF (80 mL) at RT. The resulting mixture was stirred at 60° C. for 3 h. The mixture was cooled to 10° C., quenched with saturated NH₄Cl solution (20 mL), water (200 mL) and the resulting solid was collected by filtration. The solid was washed with water (50 mL) and EtOAc (50 ml). The aqueous was extracted with EtOAc (300 mL) and the solvent was evaporated to afford a brown solid. The brown solid was triturated with water to give a solid which was collected by filtration and dried under vacuum to give crude product. The two solid batches were combined, triturated with Et₂O, collected by filtration and dried under vacuum to give the title compound (10.0 g, 68.1%) as a grey solid. ¹H NMR δ 2.64 (2H, d), 2.89 (2H, d), 3.54 (3H, s), 5.39-5.48 (1H, m), 7.07 (1H, d), 7.13 (1H, t), 7.27 (1H, d), 9.27 (1H, s), 10.41 (1H, s); m/z: ES⁺ [M+H]⁺=288.2.

### Intermediate 69d

### tert-Butyl 4-{[1-(2,6-dioxopiperidin-3-yl)-3-methyl-2-oxo-2,3-dihydro-1H-benzimidazol-4-yl]methyl}piperazine-1-carboxylate

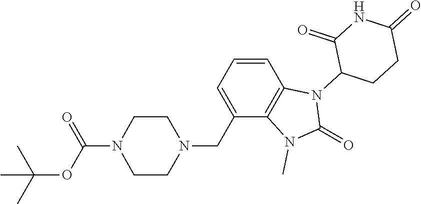 [SMILES: Cn1c(=O)n(C2CCC(=O)NC2=O)c2cccc(CN3CCN(C(=O)OC(C)(C)C)CC3)c21]

Sodium triacetoxyborohydride (3.69 g, 17.40 mmol) was added to 1-(2,6-dioxopiperidin-3-yl)-3-methyl-2-oxo-2,3-dihydro-1H-benzimidazole-4-carbaldehyde (2.0 g, 3.48 mmol) and tert-butyl piperazine-1-carboxylate (1.94 g, 10.44 mmol) in DCM:MeOH (1:2, 24 mL) and stirred at RT for 16 h. The solvent was reduced in volume, poured into NaCl Solution (25 mL), extracted with EtOAc (300 mL) and the layers were separated. The organic layer was dried over Na₂SO₄, filtered and evaporated to afford crude product. The crude product was purified by flash silica chromatography, elution gradient 50 to 100% EtOAc in Et₂O to give the title compound (0.800 g, 50.2%) as a yellow solid. ¹H NMR δ 1.40 (9H, s), 1.95-2.08 (1H, m), 2.29-2.41 (4H, m), 2.56-2.95 (3H, m), 3.24-3.32 (4H, m), 3.59-3.65 (2H, m), 3.68 (3H, s), 5.30-5.44 (1H, m), 6.86-6.93 (1H, m), 6.94-7.01 (1H, m), 7.06-7.12 (1H, m), 11.11 (1H, s); m/z: ES⁺ [M+H]⁺=458.3.

### Intermediate 69e

### 3-{3-Methyl-2-oxo-4-[(piperazin-1-yl)methyl]-2,3-dihydro-1H-benzimidazol-1-yl}piperidine-2,6-dione

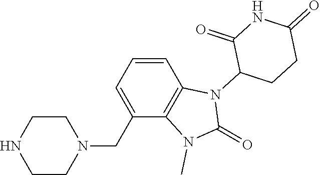 [SMILES: Cn1c(=O)n(C2CCC(=O)NC2=O)c2cccc(CN3CCNCC3)c21]

TFA (2.5 mL) was added to tert-butyl 4-{[1-(2,6-dioxopiperidin-3-yl)-3-methyl-2-oxo-2,3-dihydro-1H-benzimidazol-4-yl]methyl}piperazine-1-carboxylate (750 mg, 1.64 mmol) in DCM (10 mL) and stirred at RT for 1 h. The solvent was evaporated to give the title compound (1.1 g, 96%) as yellow oil which was used without further purification. ¹H NMR δ 1.92-2.06 (2H, m), 2.62-2.94 (9H, m), 3.65 (3H, s), 3.83-4.13 (3H, m), 5.36-5.45 (1H, m), 6.96-7.02 (2H, m), 7.15 (1H, d), 11.11 (1H, s) NH not observed; m/z: ES⁺ [M+H]⁺=358.2.

### Example 69

### 7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-methyl-1H-indole-3-carbonitrile

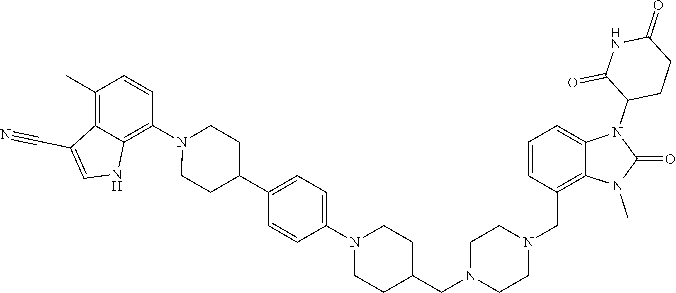 [SMILES: Cc1ccc(N2CCC(c3ccc(N4CCC(CN5CCN(Cc6cccc7c6n(C)c(=O)n7C6CCC(=O)NC6=O)CC5)CC4)cc3)CC2)c2[nH]cc(C#N)c12]

Intermediate 8b (59.3 mg, 0.13 mmol) was stirred in formic acid (2 mL) at 60° C. for 2 h and then cooled to RT. The mixture was evaporated, diluted with NMP (2 mL) and 3-{3-methyl-2-oxo-4-[(piperazin-1-yl)methyl]-2,3-dihydro-1H-benzimidazol-1-yl}piperidine-2,6-dione (45 mg, 0.13 mmol) was added followed by sodium triacetoxyborohydride (66.7 mg, 0.31 mmol) after 5 minutes. The resulting suspension was stirred at RT for 30 mins. Purification of the reaction mixture by preparative HPLC (Column A, Eluent B) gave the title compound (0.027 g, 27.9%) as beige solid. ¹H NMR (CDCl₃) δ 1.11-1.3 (2H, m), 1.53-1.68 (1H, m), 1.7-1.88 (5H, m), 1.89-2.06 (4H, m), 2.14 (2H, d), 2.44 (6H, d), 2.53-2.68 (8H, m), 2.7-2.8 (3H, m), 2.83-2.96 (1H, m), 3.37 (2H, d), 3.55-3.65 (4H, m), 3.67 (3H, s), 5.37 (1H, dd), 6.77 (1H, d), 6.84-6.92 (4H, m), 6.96 (1H, t), 7.07 (1H, d), 7.12 (2H, d), 8.15 (1H, s), 11.09 (1H, s); m/z: ES⁺ [M+H]⁺=768.4.

### Example 70

### 4-Chloro-7-[4-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile

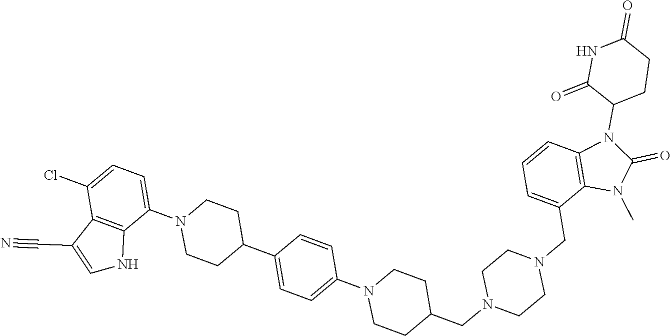 [SMILES: Cn1c(=O)n(C2CCC(=O)NC2=O)c2cccc(CN3CCN(CC4CCN(c5ccc(C6CCN(c7ccc(Cl)c8c(C#N)c[nH]c78)CC6)cc5)CC4)CC3)c21]

Intermediate 1f (35.6 mg, 0.06 mmol) and intermediate 69d (31 mg, 0.07 mmol) were heated in formic acid (1 mL, 26.51 mmol) at 60° C. for 2 h. The reaction mixture was evaporated and the residue was diluted with NMP (1 mL). The reaction mixture was stirred for 5 mins followed by the addition of sodium triacetoxyhydroborate (32.6 mg, 0.15 mmol). The resulting suspension was stirred at RT for 2 h. Purification of the reaction mixture by preparative HPLC (Column A, Eluent B) gave fractions containing the desired compound which were extracted with DCM (100 mL), dried over MgSO₄, filtered and evaporated to dryness to give the title compound (9.0 mg, 18.53%) as a white solid. ¹H NMR δ 1.19 (2H, t), 1.25 (2H, s), 1.61 (1H, s), 1.78 (2H, d), 1.84 (2H, d), 1.91-2.05 (3H, m), 2.11-2.2 (2H, m), 2.38-2.47 (4H, m), 2.54-2.63 (4H, m), 2.66 (1H, s), 2.66-2.84 (4H, m), 2.85-2.97 (1H, m), 3.42 (2H, d), 3.59-3.66 (4H, m), 3.68 (3H, s), 5.38 (1H, dd), 6.84-6.93 (4H, m), 6.97 (1H, t), 7.08 (1H, d), 7.15 (3H, dd), 8.32 (1H, d), 11.09 (1H, s), 12.26 (1H, s); m/z: ES⁺ [M+H]⁺=788.6.

### Example 71

### 7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile

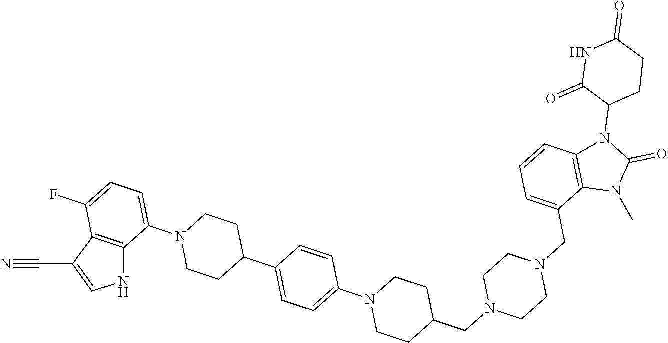 [SMILES: Cn1c(=O)n(C2CCC(=O)NC2=O)c2cccc(CN3CCN(CC4CCN(c5ccc(C6CCN(c7ccc(F)c8c(C#N)c[nH]c78)CC6)cc5)CC4)CC3)c21]

The title compound was prepared using methodology described in example 70 using intermediate 67d and intermediate 2c to give the title compound (20 mg, 23%) as a white solid. ¹H NMR (90° C.) S 1.17-1.37 (2H, m), 1.56-1.73 (1H, m), 1.77-1.93 (4H, m), 1.93-2.02 (2H, m), 2.02-2.11 (1H, m), 2.15-2.25 (2H, m), 2.38-2.43 (4H, m), 2.44-2.48 (4H, m), 2.63-2.74 (4H, m), 2.78-2.99 (4H, m), 3.40 (2H, d), 3.61 (2H, d), 3.67-3.71 (5H, m), 5.26-5.37 (1H, m), 6.82-7.09 (7H, m), 7.14 (2H, d), 8.10 (1H, s), 8.21 (1H, s), 10.71 (1H, s); m/z: ES⁺ [M+H]⁺=772.9.

### Example 72

### Intermediate 72a

### 3-(4-Bromo-3-methyl-2-oxo-2,3-dihydro-1H-benzimidazol-1-yl)piperidine-2,6-dione

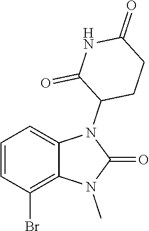 [SMILES: Cn1c(=O)n(C2CCC(=O)NC2=O)c2cccc(Br)c21]

The title compound was prepared using methodology described in intermediate 69c using 7-bromo-1-methyl-1,3-dihydro-2H-benzimidazol-2-one and 1-bromo-1,3-diazinane-2,4-dione to give the title compound (743 mg, 50%) as a grey solid. ¹H NMR (CDCl₃) δ 2.23 (1H, m), 2.67-3 (3H, m), 3.79 (3H, s), 5.19 (1H, dd), 6.73 (1H, dd), 6.92 (1H, t), 7.24 (1H, dd), 8.08 (1H, s); m/z: ES⁺ [M+H]⁺=338.0.

### Intermediate 72b

### tert-Butyl 4-[1-(2,6-dioxopiperidin-3-yl)-3-methyl-2-oxo-2,3-dihydro-1H-benzimidazol-4-yl]piperazine-1-carboxylate

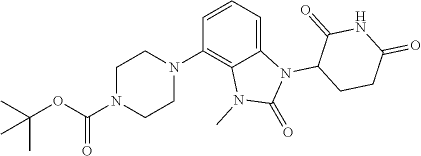 [SMILES: Cn1c(=O)n(C2CCC(=O)NC2=O)c2cccc(N3CCN(C(=O)OC(C)(C)C)CC3)c21]

3-(4-Bromo-3-methyl-2-oxo-2,3-dihydro-1H-benzimidazol-1-yl)piperidine-2,6-dione (2.21 g, 6.55 mmol), Pd-PEPPSI-IPent (0.550 g, 0.65 mmol), tert-butyl piperazine-1-carboxylate (3.05 g, 16.37 mmol) and sodium tert-butoxide (1.88 g, 19.64 mmol) were suspended in 1,4-dioxane (60 mL) under nitrogen. The reaction was stirred at 50° C. for 1 h. The reaction was cooled to RT, diluted with DCM (100 mL), washed with NH₄Cl solution (50 mL), water (50 mL), NaHCO₃ solution (50 mL) and NaCl solution (50 mL). The organic layer was dried with Na₂SO₄, filtered and evaporated to afford crude product. The crude product was triturated with Hexane:EtOAc (1:1, 100 mL) and filtered under vacuum. The solid was then slurried in EtOH (50 mL) for 20 mins. The solid was filtered under vacuum and then slurried in MeCN (50 mL). The solid was filtered under vacuum to give the title compound (1.48 g, 51.2%) as a beige solid. ¹H NMR δ 1.42 (9H, s), 1.98 (1H, br t), 2.56-2.76 (4H, m), 2.79-2.96 (2H, m), 2.96-3.20 (4H, m), 3.62 (3H, s), 3.94 (1H, br s), 5.30-5.40 (1H, m), 6.88-6.94 (2H, m), 6.94-7.00 (1H, m), 11.09 (1H, br s); m/z: ES⁺ [M+Na]=466.1.

### Example 72

### 4-Chloro-7-(4-{4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]phenyl}piperidin-1-yl)-1H-indole-3-carbonitrile

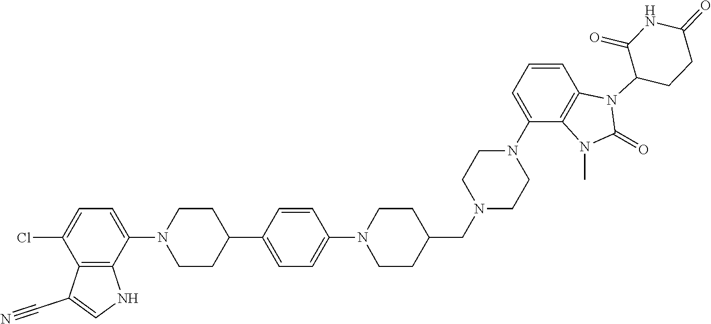 [SMILES: Cn1c(=O)n(C2CCC(=O)NC2=O)c2cccc(N3CCN(CC4CCN(c5ccc(C6CCN(c7ccc(Cl)c8c(C#N)c[nH]c78)CC6)cc5)CC4)CC3)c21]

The title compound was prepared using methodology described in example 7 using tert-butyl 4-[1-(2,6-dioxopiperidin-3-yl)-3-methyl-2-oxo-2,3-dihydro-1H-benzimidazol-4-yl]piperazine-1-carboxylate and intermediate 1f to give the title compound (60 mg, 47%) as a white solid. ¹H NMR δ 1.18-1.31 (3H, m), 1.63-1.76 (1H, m), 1.76-1.88 (4H, m), 1.89-2.06 (4H, m), 2.22-2.35 (3H, m), 2.55-2.65 (4H, m), 2.68-2.83 (3H, m), 2.83-3.06 (6H, m), 3.42 (2H, d), 3.59-3.7 (5H, m), 5.35 (1H, dd), 6.8-7.05 (6H, m), 7.15 (3H, dd), 8.31 (1H, d), 11.08 (1H, s), 12.25 (1H, s); m/z: ES⁺ [M+H]⁺=774.4.

### Example 73

### 4-Chloro-7-[(3S)-3-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile

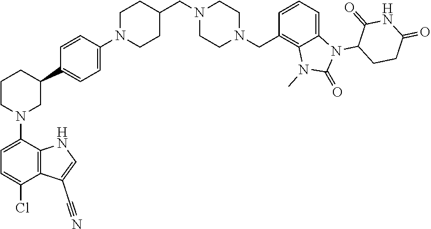 [SMILES: Cn1c(=O)n(C2CCC(=O)NC2=O)c2cccc(CN3CCN(CC4CCN(c5ccc([C@@H]6CCCN(c7ccc(Cl)c8c(C#N)c[nH]c78)C6)cc5)CC4)CC3)c21]

The title compound was prepared using methodology described in example 69 using intermediate 11c and intermediate 69d to give the title compound (6.0 mg, 5%) as a white solid. ¹H NMR δ 1.07-1.22 (2H, m), 1.24 (1H, s), 1.56 (2H, d), 1.76 (2H, d), 1.96 (4H, dt), 2.14 (2H, d), 2.27-2.47 (7H, m), 2.51-2.77 (7H, m), 2.79-3.09 (2H, m), 3.32 (1H, s), 3.47-3.7 (7H, m), 5.38 (1H, dd), 6.8-7.01 (5H, m), 7.03-7.2 (4H, m), 8.31 (1H, d), 11.10 (1H, s), 12.29 (1H, s); m/z: ES⁺ [M+H]⁺=788.4.

### Example 74

### 7-[(3S)-3-(4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile

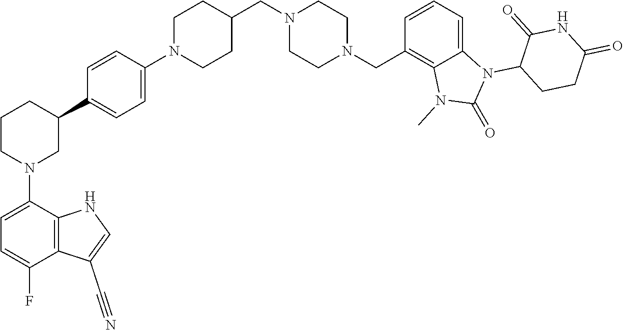 [SMILES: Cn1c(=O)n(C2CCC(=O)NC2=O)c2cccc(CN3CCN(CC4CCN(c5ccc([C@@H]6CCCN(c7ccc(F)c8c(C#N)c[nH]c78)C6)cc5)CC4)CC3)c21]

The title compound was prepared using methodology described in example 69 using intermediate 23a and intermediate 69d to give the title compound (26 mg, 19%) as a white solid. ¹H NMR δ 1.07-1.27 (2H, m), 1.46-1.61 (2H, m), 1.75 (2H, d), 1.8-2.05 (5H, m), 2.12 (2H, d), 2.37 (6H, d), 2.52-2.64 (5H, m), 2.64-2.77 (2H, m), 2.82-2.91 (1H, m), 2.99 (1H, t), 3.28 (2H, d), 3.53-3.72 (7H, m), 5.37 (1H, dd), 6.75-6.98 (5H, m), 7.07 (1H, d), 7.15 (2H, d), 8.25 (1H, s), 11.10 (1H, s), 12.25 (1H, s); m/z: ES⁺ [M+H]⁺=772.4.

### Example 75

### Intermediate 75a

### tert-Butyl diprop-2-yn-1-ylcarbamate

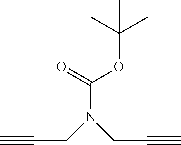

To tert-butyl prop-2-yn-1-ylcarbamate (5.0 g, 32.22 mmol) in DMF (150 mL) under nitrogen at 0° C. was added sodium hydride (60% in mineral oil, 2.58 g, 64.43 mmol) and stirred for 20 mins. 3-Bromoprop-1-yne (80% in toluene, 7.18 ml, 64.43 mmol) was added dropwise and the reaction mixture was stirred to RT for 16 h. The reaction was quenched with ice and extracted with Et₂O (300 mL). The organic layer was washed with water (2×200 mL), NaCl solution (100 mL) and dried over a phase separating cartridge. The solvent was evaporated to give the title compound (2.71 g, 43.5%) as a yellow oil. ¹H NMR δ 1.42 (9H, s), 3.22 (2H, t), 4.05 (4H, d). no mass ion data

### Intermediate 75b

### 2-tert-Butyl 5,6-dimethyl 1,3-dihydro-2H-isoindole-2,5,6-tricarboxylate

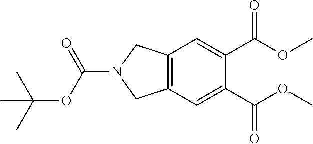 [SMILES: COC(=O)c1cc2c(cc1C(=O)OC)CN(C(=O)OC(C)(C)C)C2]

tert-Butyl diprop-2-yn-1-ylcarbamate (2.71 g, 14.02 mmol) and dimethyl but-2-ynedioate (6.90 ml, 56.09 mmol) in EtOH (30 mL) was degassed using nitrogen for 10 minutes. Chlorotris(triphenylphosphine)rhodium(I) (0.260 g, 0.28 mmol) was then added and the reaction was heated to 80° C. for 48 h. The reaction was cooled to RT and the solvent was evaporated. Et₂O (200 mL) was added and the resulting solid was filtered under vacuum to afford crude product. The crude product was purified by flash silica chromatography, elution gradient 0 to 100% EtOAc in heptane to give the title compound (1.10 g, 23.39%) as a yellow oil. ¹H NMR δ 1.47 (9H, s), 3.82 (6H, s), 4.66 (4H, d), 7.71 (2H, d); m/z: ES⁻ [M−H]⁻=334.0.

### Intermediate 75c

### 2-(tert-Butoxycarbonyl)-2,3-dihydro-1H-isoindole-5,6-dicarboxylic acid

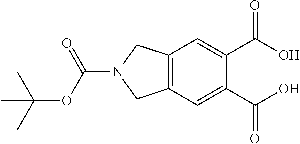 [SMILES: CC(C)(C)OC(=O)N1Cc2cc(C(=O)O)c(C(=O)O)cc2C1]

To 2-tert-butyl 5,6-dimethyl 1,3-dihydro-2H-isoindole-2,5,6-tricarboxylate (1.1 g, 3.28 mmol) in MeOH (10 mL) and water (2.5 mL) was added sodium hydroxide (2M, 4.92 ml, 9.84 mmol) and stirred at 80° C. for 2 h.

The solvent was removed under reduced pressure and the residue was acidified to pH 4 using HCl (2M). The product was extracted with EtOAc (100 mL), washed with NaCl solution (50 mL), dried over a phase separating cartridge and evaporated to give the title compound (0.452 g, 44.8%) as a yellow solid which was used without further purification. ¹H NMR δ 1.47 (9H, s), 4.64 (4H, d), 7.67 (2H, d), 13.22 (2H, s). m/z: ES⁻ [M−H]⁻=306.0.

### Intermediate 75d

### tert-Butyl 1,3-dioxo-5,7-dihydro-1H-furo[3,4-f]isoindole-6(3H)-carboxylate

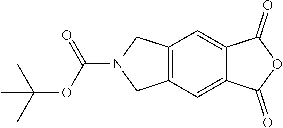 [SMILES: CC(C)(C)OC(=O)N1Cc2cc3c(cc2C1)C(=O)OC3=O]

2-(tert-Butoxycarbonyl)-2,3-dihydro-1H-isoindole-5,6-dicarboxylic acid (452 mg, 1.47 mmol) was stirred at 100° C. in acetic anhydride (3 mL, 31.74 mmol) for 2 h. The reaction mixture was cooled to RT, diluted with EtOAc (50 mL), washed with water (2×50 mL) and NaCl solution (50 mL). The organic layer was dried with a phase separating cartridge, filtered and evaporated to afford crude product. The crude product was purified by flash silica chromatography, elution gradient 0 to 100% EtOAc in heptane to give the title compound (0.318 g, 74.7%) as a yellow solid. ¹H NMR δ 1.48 (9H, s), 4.75 (4H, d), 8.05 (2H, d); m/z: ES⁺ [M+H]⁺=290.1.

### Intermediate 75e

### tert-Butyl 6-(2,6-dioxopiperidin-3-yl)-5,7-dioxo-3,5,6,7-tetrahydropyrrolo[3,4-f]isoindole-2(1H)-carboxylate

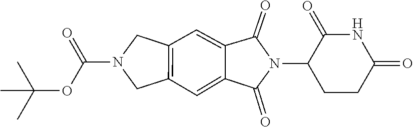 [SMILES: CC(C)(C)OC(=O)N1Cc2cc3c(cc2C1)C(=O)N(C1CCC(=O)NC1=O)C3=O]

To tert-butyl 1,3-dioxo-5,7-dihydro-1H-furo[3,4-f]isoindole-6(3H)-carboxylate (318 mg, 1.10 mmol) in toluene (5 mL) was added 3-aminopiperidine-2,6-dione hydrochloride (199 mg, 1.21 mmol) and triethylamine (460 μl, 3.30 mmol). The reaction was heated to 80° C. for 3 h and was then cooled to RT, diluted with EtOAc (50 mL), washed with water (2×50 mL), NaCl solution (20 mL) and dried over a phase separating cartridge. The solvent was evaporated to afford crude product. The crude product was purified by flash silica chromatography, elution gradient 0 to 100% EtOAc in heptane to give the title compound (0.290 g, 66.1%) as a white solid. ¹H NMR δ 1.54 (9H, s), 2.1-2.18 (1H, m), 2.65-2.74 (2H, m), 2.88-3.04 (1H, m), 4.79 (4H, d), 5.13-5.31 (1H, m), 7.96 (2H, s), 11.17 (1H, s); m/z: ES⁻[M−H]⁻=398.3.

### Intermediate 75f

### 2-(2,6-Dioxopiperidin-3-yl)-6,7-dihydropyrrolo[3,4-f]isoindole-1,3(2H,5H)-dione

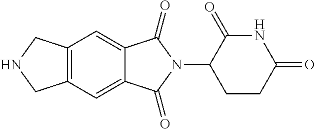 [SMILES: O=C1CCC(N2C(=O)c3cc4c(cc3C2=O)CNC4)C(=O)N1]

HCl (4M in dioxane, 1 mL, 4.27 mmol) was added to tert-butyl 6-(2,6-dioxopiperidin-3-yl)-5,7-dioxo-3,5,6,7-tetrahydropyrrolo[3,4-f]isoindole-2(1H)-carboxylate (284 mg, 0.71 mmol) and the mixture was stirred at RT for 2 h. The solvent was evaporated to give the title compound (0.149 g, 62.4%) as a white solid and was used without further purification. ¹H NMR δ 2.03-2.17 (2H, m), 2.83-2.98 (1H, m), 3.58 (1H, s), 4.66 (4H, s), 5.1-5.22 (1H, m), 7.98 (2H, s), 9.67 (1H, s), 11.14 (1H, s); m/z: ES⁺ [M+H]⁺=300.3.

### Example 75

### 4-Chloro-7-{4-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile

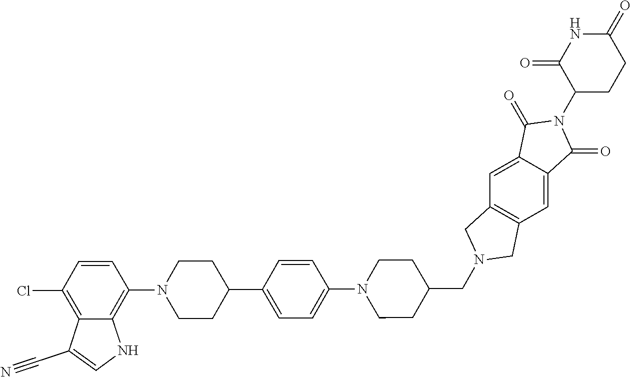 [SMILES: N#Cc1c[nH]c2c(N3CCC(c4ccc(N5CCC(CN6Cc7cc8c(cc7C6)C(=O)N(C6CCC(=O)NC6=O)C8=O)CC5)cc4)CC3)ccc(Cl)c12]

The title compound was prepared using methodology described in example 23 using intermediate if and 2-(2,6-dioxopiperidin-3-yl)-6,7-dihydropyrrolo[3,4-f]isoindole-1,3(2H,5H)-dione to give the title compound in the form of a formate salt (44 mg, 36%) as a pale yellow solid. ¹H NMR (90° C.) δ 1.24-1.45 (3H, m), 1.67-1.78 (1H, m), 1.85-1.93 (4H, m), 1.95-2.04 (2H, m), 2.07-2.16 (1H, m), 2.62-2.69 (4H, m), 2.7-2.79 (2H, m), 2.81-2.93 (4H, m), 3.45 (2H, d), 3.65 (2H, d), 4.05 (4H, s), 4.99-5.19 (1H, m), 6.79-6.96 (3H, m), 7.1-7.2 (3H, m), 7.76 (2H, s), 8.16 (1H, s); m/z: ES⁺ [M+H]⁺=730.8.

### Example 76

### 4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indazole-3-carbonitrile

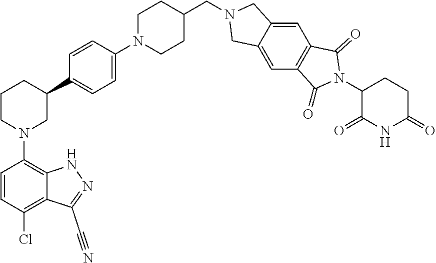 [SMILES: N#Cc1n[nH]c2c(N3CCC[C@@H](c4ccc(N5CCC(CN6Cc7cc8c(cc7C6)C(=O)N(C6CCC(=O)NC6=O)C8=O)CC5)cc4)C3)ccc(Cl)c12]

The title compound was prepared using methodology described in example 7 using intermediate 9g and intermediate 75f to give the title compound (38 mg, 16%) as a white solid. ¹H NMR δ 1.17-1.36 (2H, m), 1.47-1.7 (2H, m), 1.75-1.99 (5H, m), 2.01-2.14 (1H, m), 2.51-2.8 (8H, m), 2.82-3.06 (2H, m), 3.47 (2H, t), 3.64 (2H, d), 3.99 (4H, s), 5.14 (1H, dd), 6.91 (3H, dd), 7.18 (2H, d), 7.27 (1H, d), 7.79 (2H, s), 11.12 (1H, s), 14.69 (1H, s); m/z: ES⁺ [M+H]⁺=731.0.

### Example 77

### 4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile

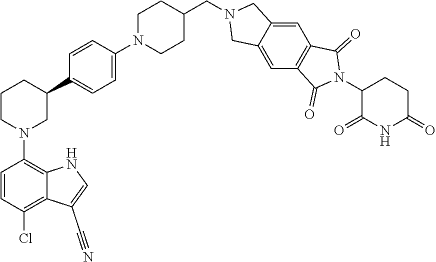 [SMILES: N#Cc1c[nH]c2c(N3CCC[C@@H](c4ccc(N5CCC(CN6Cc7cc8c(cc7C6)C(=O)N(C6CCC(=O)NC6=O)C8=O)CC5)cc4)C3)ccc(Cl)c12]

The title compound was prepared using methodology described in example 7 using intermediate 11c and intermediate 75f to give the title compound (36 mg, 28%) as a white solid. ¹H NMR δ 1.03-1.37 (2H, m), 1.49-1.72 (2H, m), 1.78-1.98 (5H, m), 2.01-2.14 (1H, m), 2.53-2.77 (8H, m), 2.82-3.11 (2H, m), 3.27-3.32 (1H, m), 3.37 (1H, s), 3.64 (2H, d), 3.97 (4H, s), 5-5.24 (1H, m), 6.71-6.97 (3H, m), 7.04-7.29 (3H, m), 7.79 (2H, s), 8.32 (1H, s), 11.14 (1H, s), 12.29 (1H, s); m/z: ES⁺ [M+H]⁺=730.0.

### Example 78

### Intermediate 78a

### tert-Butyl 5-(3-cyano-4-methyl-1H-indol-7-yl)-3,6-dihydropyridine-1(2H)-carboxylate

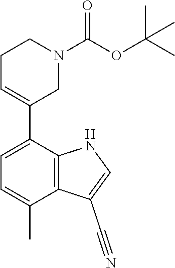 [SMILES: Cc1ccc(C2=CCCN(C(=O)OC(C)(C)C)C2)c2[nH]cc(C#N)c12]

The title compound was prepared using methodology described in intermediate 63b using intermediate 8a and tert-butyl 5-(4,4,5,5-tetramethyl-1,3,2-dioxaborolan-2-yl)-3,4-dihydropyridine-1(2H)-carboxylate to give the title compound (1.9 g, 99%) as a brown solid. ¹H NMR δ 1.42 (9H, s), 2.25-2.35 (2H, m), 2.65 (3H, s), 3.54 (2H, t), 4.12 (2H, q), 6.02-6.11 (1H, m), 6.92-7.07 (2H, m), 8.23 (1H, d), 11.96 (1H, s); m/z: ES⁺ [M+H]⁺=338.3.

### Intermediate 78b

### tert-Butyl 3-(3-cyano-4-methyl-1H-indol-7-yl)piperidine-1-carboxylate

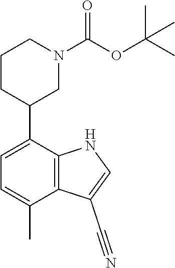 [SMILES: Cc1ccc(C2CCCN(C(=O)OC(C)(C)C)C2)c2[nH]cc(C#N)c12]

The title compound was prepared using methodology described in intermediate 63c using tert-butyl 5-(3-cyano-4-methyl-1H-indol-7-yl)-3,6-dihydropyridine-1(2H)-carboxylate to give the title compound (2.8 g, 69%) as a white foam. ¹H NMR δ 1.39 (9H, s), 1.49-1.65 (2H, m), 1.67-1.76 (1H, m), 1.88-1.97 (1H, m), 2.62 (3H, s), 2.74-2.88 (1H, m), 2.94-3.16 (2H, m), 3.95-4.06 (2H, m), 6.93 (1H, d), 7.03 (1H, d), 8.25 (1H, d), 12.19 (1H, s); m/z: ES⁺ [M+H]⁺=340.3.

### Intermediate 78c

### 4-Methyl-7-(piperidin-3-yl)-1H-indole-3-carbonitrile

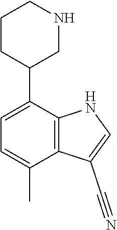 [SMILES: Cc1ccc(C2CCCNC2)c2[nH]cc(C#N)c12]

Formic acid (30 mL) was added to tert-butyl 3-(3-cyano-4-methyl-1H-indol-7-yl)piperidine-1-carboxylate (2.8 g, 8.25 mmol) at RT and was stirred for 3 h. The solvent was evaporated and the crude product was purified by ion exchange chromatography, using an SCX column. The desired product was eluted from the column using 7M NH₃:MeOH to give the title compound (1.9 g, 96%) as a white foam. ¹H NMR (CDCl₃) δ 1.50-1.69 (2H, m), 1.83-2.07 (3H, m), 2.76 (3H, s), 2.87-2.99 (1H, m), 3.11-3.27 (3H, m), 3.27-3.35 (1H, m), 6.93 (2H, d), 7.72 (1H, s) one proton exchanged; m/z: ES⁺ [M+H]⁺=240.1.

### Intermediate 78d

### 2-[3-(4-Bromophenoxy)propyl]-1,3-dioxolane

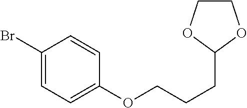 [SMILES: Brc1ccc(OCCCC2OCCO2)cc1]

2-(3-Bromopropyl)-1,3-dioxolane (1.69 g, 8.67 mmol) was added to 4-bromophenol (1.0 g, 5.78 mmol) and K₂CO₃ (2.39 g, 17.34 mmol) in MeCN (20 mL) and the mixture was stirred at 80° C. for 2 h. The reaction was cooled to RT and the solvent was evaporated to afford crude product which was purified by flash silica chromatography, elution gradient 0 to 100% DCM in Et₂O to give the title compound (1.5 g, 90%) as a colourless oil. ¹H NMR δ 1.65-1.77 (4H, m), 3.73-3.87 (4H, m), 3.94-4.10 (2H, m), 4.78-4.89 (1H, m), 6.89 (2H, d), 7.43 (2H, d); m/z: ES⁺ [M+H]⁺=287.1.

### Intermediate 78e and 79a

The title compounds were prepared using methodology described in intermediate 11c using 4-methyl-7-(piperidin-3-yl)-1H-indole-3-carbonitrile and 2-[3-(4-bromophenoxy)propyl]-1,3-dioxolane to give a racemic mixture. The enantiomers were separated by preparative chiral-HPLC on a Column: CHIRAL ART Cellulose-SB, 2*25 cm, 5 μm; Mobile Phase A: Hex (0.5% 2M NH3-MeOH), Mobile Phase B: EtOH; Flow rate: 20 mL/min; Gradient: 25% B in 10.5 mins to give, in order of elution, intermediate 78e (isomer 1, 86 mg, 13%) and intermediate 79a (isomer 2, 77 mg, 11%) as yellow foams.

### Intermediate 78e

### 7-[(3R*)-1-{4-[3-(1,3-dioxolan-2-yl)propoxy]phenyl}piperidin-3-yl]-4-methyl-1H-indole-3-carbonitrile [Absolute Stereochemistry not Yet Confirmed]

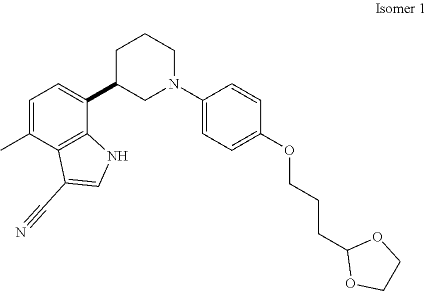 [SMILES: Cc1ccc([C@H]2CCCN(c3ccc(OCCCC4OCCO4)cc3)C2)c2[nH]cc(C#N)c12]

¹H NMR δ 1.42-1.89 (8H, m), 2.51 (3H, d), 2.55-2.69 (1H, m), 3.24-3.3 (2H, m), 3.36-3.47 (2H, m), 3.58-3.68 (2H, m), 3.68-3.81 (4H, m), 4.71 (1H, d), 6.61-6.71 (2H, m), 6.73-6.86 (3H, m), 6.93-7.02 (1H, m), 8.08 (1H, t), 12.14 (1H, s); m/z: ES⁺ [M+H]⁺=446.2; >99% ee.

### Intermediate 79a

### 7-[(3S*)-1-{4-[3-(1,3-dioxolan-2-yl)propoxy]phenyl}piperidin-3-yl]-4-methyl-1H-indole-3-carbonitrile [Absolute Stereochemistry not Yet Confirmed]

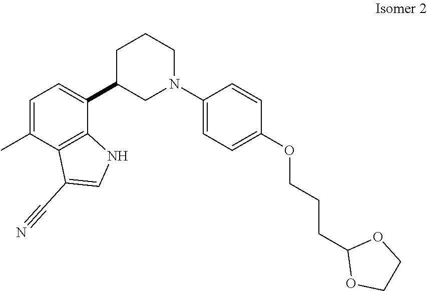 [SMILES: Cc1ccc([C@H]2CCCN(c3ccc(OCCCC4OCCO4)cc3)C2)c2[nH]cc(C#N)c12]

¹H NMR δ 1.47-1.79 (8H, m), 2.51 (3H, s), 2.55-2.69 (1H, m), 3.24-3.34 (2H, m), 3.4-3.46 (2H, m), 3.58-3.68 (2H, m), 3.68-3.82 (4H, m), 4.71 (1H, t), 6.62-6.71 (2H, m), 6.74-6.86 (3H, m), 6.98 (1H, d), 8.09 (1H, d), 12.14 (1H, s); m/z: ES⁺ [M+H]⁺=446.3; >99% ee.

### Example 78

### 7-{(3R*)-1-[4-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile [Absolute Stereochemistry not Yet Confirmed]

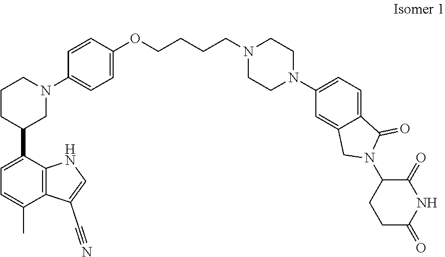 [SMILES: Cc1ccc([C@H]2CCCN(c3ccc(OCCCCN4CCN(c5ccc6c(c5)CN(C5CCC(=O)NC5=O)C6=O)CC4)cc3)C2)c2[nH]cc(C#N)c12]

The title compound was prepared using methodology described in example 24 using intermediate 78e and intermediate 1i followed by purification by flash C18-flash chromatography, elution gradient 0 to 100% MeCN in water modified with 0.1% NH₄HCO₃ followed by purification by preparative TLC DCM:MeOH (14:1) to give the title compound (32 mg, 25%) as a white solid. ¹H NMR δ 1.52-1.74 (5H, m), 1.81-1.87 (2H, m), 1.89-1.98 (2H, m), 2.38 (3H, q), 2.49 (4H, s), 2.55 (1H, s), 2.63 (5H, s), 2.67-2.8 (1H, m), 2.81-2.93 (1H, m), 3.21-3.29 (4H, m), 3.52-3.58 (2H, m), 3.90 (2H, t), 4.12-4.38 (2H, m), 5.05 (1H, dd), 6.75-6.85 (2H, m), 6.86-6.98 (3H, m), 7.01-7.14 (3H, m), 7.52 (1H, d), 8.22 (1H, d), 10.96 (1H, s), 12.27 (1H, d); m/z: ES⁺ [M+H]⁺=714.4.

### Example 79

### 7-{(3S*)-1-[4-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile [Absolute Stereochemistry not Yet Confirmed]

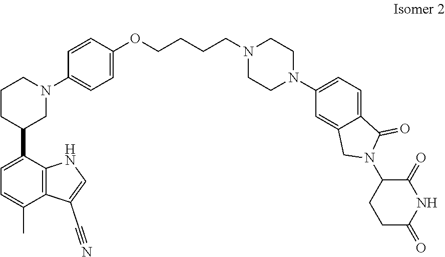 [SMILES: Cc1ccc([C@H]2CCCN(c3ccc(OCCCCN4CCN(c5ccc6c(c5)CN(C5CCC(=O)NC5=O)C6=O)CC4)cc3)C2)c2[nH]cc(C#N)c12]

The title compound was prepared using methodology described in example 24 using intermediate 79a and intermediate 1i followed by purification by flash C18-flash chromatography, elution gradient 0 to 100% MeCN in water modified with 0.1% NH₄HCO₃ followed by purification by preparative TLC DCM:MeOH (14:1) to give the title compound (21 mg, 18%) as a white solid. ¹H NMR δ 1.52-1.76 (5H, m), 1.79-1.99 (4H, m), 2.36 (3H, t), 2.46-2.49 (4H, m), 2.56 (1H, s), 2.59-2.66 (5H, m), 2.67-2.8 (1H, m), 2.81-2.93 (1H, m), 3.24-3.3 (4H, m), 3.5-3.6 (2H, m), 3.90 (2H, t), 4.13-4.37 (2H, m), 5.05 (1H, dd), 6.75-6.85 (2H, m), 6.86-6.98 (3H, m), 7.01-7.14 (3H, m), 7.52 (1H, d), 8.22 (1H, d), 10.96 (1H, s), 12.27 (1H, s); m/z: ES⁺ [M+H]⁺=714.4.

### Example 80

### Intermediate 80a

### 2-[3-(3-Bromophenoxy)propyl]-1,3-dioxolane

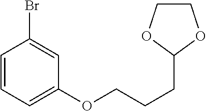 [SMILES: Brc1cccc(OCCCC2OCCO2)c1]

The title compound was prepared using methodology described in intermediate 78d using 3-bromophenol to give the title compound (1.2 g, 72%) as a colourless gum. ¹H NMR δ 1.65-1.83 (5H, m), 1.81-1.96 (1H, m), 3.56 (1H, td), 3.79 (1H, d), 3.89 (1H, s), 4.01 (1H, td), 4.79-4.89 (1H, m), 6.94 (1H, dt), 7.07-7.15 (2H, m), 7.23 (1H, m); m/z: ES⁺ [M+H]⁺=287.0.

### Intermediate 80b

### 7-[(3R*)-1-{3-[3-(1,3-dioxolan-2-yl)propoxy]phenyl}piperidin-3-yl]-4-methyl-1H-indole-3-carbonitrile [Absolute Stereochemistry not Yet Confirmed]

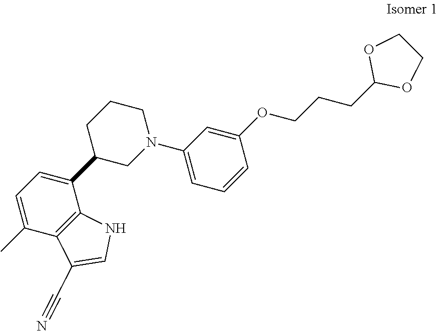 [SMILES: Cc1ccc([C@H]2CCCN(c3cccc(OCCCC4OCCO4)c3)C2)c2[nH]cc(C#N)c12]

### and

### Intermediate 81a

### 7-[(3S*)-1-{3-[3-(1,3-dioxolan-2-yl)propoxy]phenyl}piperidin-3-yl]-4-methyl-1H-indole-3-carbonitrile [Absolute Stereochemistry not Yet Confirmed]

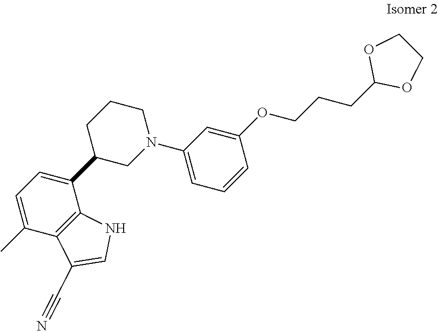 [SMILES: Cc1ccc([C@H]2CCCN(c3cccc(OCCCC4OCCO4)c3)C2)c2[nH]cc(C#N)c12]

The title compounds were prepared using methodology described in intermediate 78e and intermediate 79a using 4-methyl-7-(piperidin-3-yl)-1H-indole-3-carbonitrile and 2-[3-(3-bromophenoxy)propyl]-1,3-dioxolane to give a racemic mixture. The enantiomers were separated by preparative chiral-HPLC on a Column: CHIRAL ART Cellulose-SB, 2*25 cm, 5 μm; Mobile Phase A: Hex (0.5% 2M NH3-MeOH), Mobile Phase B: EtOH; Flow rate: 20 m/min; Gradient: 30% B in 10.5 mins to give, in order of elution, intermediate 80b (isomer 1, 138 mg, 45%, >99% ee) and intermediate 81a (isomer 2, 120 mg, 42%, >99% ee) as yellow solids. No nmr or lcms data collected at this stage.

### Example 80

### 7-{(3R*)-1-[3-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile [Absolute Stereochemistry not Yet Confirmed]

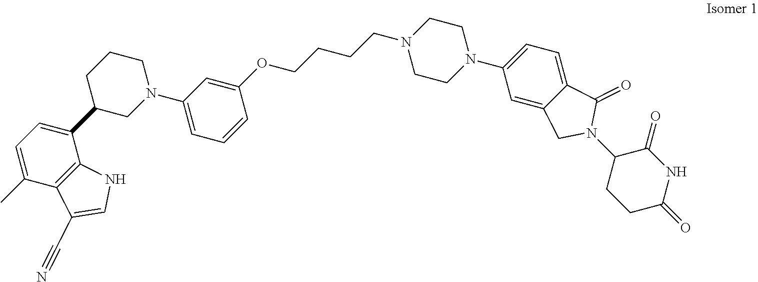 [SMILES: Cc1ccc([C@H]2CCCN(c3cccc(OCCCCN4CCN(c5ccc6c(c5)CN(C5CCC(=O)NC5=O)C6=O)CC4)c3)C2)c2[nH]cc(C#N)c12]

The title compound was prepared using methodology described in example 24 using intermediate 80b and intermediate 1i and purified by flash C18-flash chromatography, elution gradient 0 to 90% MeCN in water (0.1% NH₄HCO₃) to give the title compound (27 mg, 12%) as a white solid. ¹H NMR δ 1.59 (3H, s), 1.70 (3H, s), 1.81 (2H, s), 1.96 (2H, s), 2.31-2.39 (4H, m), 2.63 (5H, s), 2.78 (2H, s), 2.88-2.93 (3H, m), 3.26 (4H, s), 3.74 (2H, t), 3.9-3.97 (2H, m), 4.16-4.25 (1H, m), 4.32 (1H, d), 5.02-5.1 (1H, m), 6.34 (1H, d), 6.46 (1H, d), 6.54 (1H, d), 6.95 (1H, t), 7.01-7.13 (4H, m), 7.52 (1H, t), 8.23 (1H, d), 10.96 (1H, s), 12.27 (1H, s); m/z: ES⁺ [M+H]⁺=714.4.

### Example 81

### 7-{(3S*)-1-[3-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile [Absolute Stereochemistry not Yet Confirmed]

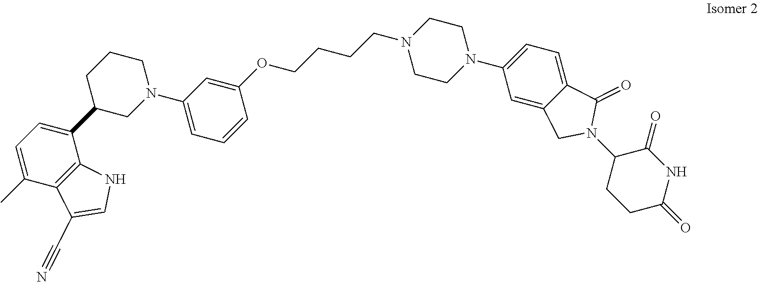 [SMILES: Cc1ccc([C@H]2CCCN(c3cccc(OCCCCN4CCN(c5ccc6c(c5)CN(C5CCC(=O)NC5=O)C6=O)CC4)c3)C2)c2[nH]cc(C#N)c12]

The title compound was prepared using methodology described in example 24 using intermediate 81a and intermediate 1i and purified by flash C18-flash chromatography, elution gradient 0 to 90% MeCN in water (0.1% NH₄HCO₃) to give the title compound (11 mg, 6%) as a white solid. ¹H NMR δ 1.54-1.64 (2H, m), 1.64-1.74 (2H, m), 1.80 (2H, s), 1.91-1.99 (2H, m), 2.31-2.41 (3H, m), 2.44-2.5 (4H, m), 2.53-2.65 (5H, m), 2.75-2.79 (1H, m), 2.84-2.97 (2H, m), 3.25 (4H, t), 3.74 (2H, t), 3.94 (2H, t), 4.20 (1H, d), 4.32 (1H, d), 5.05 (1H, dd), 6.3-6.37 (1H, m), 6.45 (1H, t), 6.5-6.57 (1H, m), 6.94 (1H, d), 7-7.13 (4H, m), 7.52 (1H, d), 8.23 (1H, d), 10.96 (1H, s), 12.26 (1H, s); m/z: ES⁺ [M+H]⁺=714.4.

### Example 82

### Intermediate 82a

### 1-(4-Bromophenyl)-4-(1,3-dioxolan-2-yl)piperidine

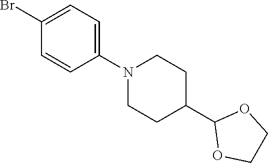 [SMILES: Brc1ccc(N2CCC(C3OCCO3)CC2)cc1]

Pd₂(dba)₃ (175 mg, 0.19 mmol) was added to Xantphos (110 mg, 0.19 mmol), Cs₂CO₃ (3.1 g, 9.54 mmol), 1-bromo-4-iodobenzene (1.1 g, 4.20 mmol) and 4-(1,3-dioxolan-2-yl)piperidine (600 mg, 3.82 mmol) in 1,4-dioxane (20 mL) under nitrogen. The mixture was stirred at 80° C. for 3 h and then cooled to RT. The solvent was evaporated to afford crude product. The crude product was purified by flash silica chromatography, elution gradient 0 to 55% EtOAc in Et₂O to give the title compound (0.30 g, 25.2%) as a yellow solid. ¹H NMR δ 1.30-1.44 (2H, m), 1.59-1.76 (3H, m), 2.58-2.69 (2H, m), 3.66-3.74 (2H, m), 3.74-3.84 (2H, m), 3.81-3.93 (2H, m), 4.56-4.63 (1H, m), 6.84-6.91 (2H, m), 7.27-7.36 (2H, m); m/z: ES⁺ [M+H]⁺=312.1.

### Intermediate 82b

### 7-[(3R*)-1-{4-[4-(1,3-dioxolan-2-yl)piperidin-1-yl]phenyl}piperidin-3-yl]-4-methyl-1H-indole-3-carbonitrile [Absolute Stereochemistry not Yet Confirmed]

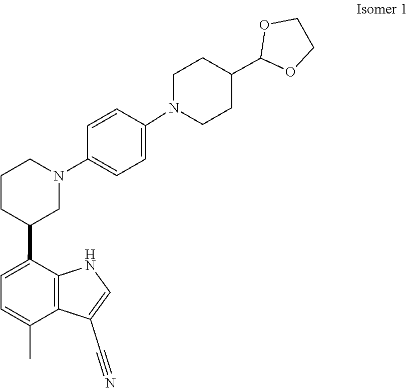

### and

### Intermediate 83a

### 7-[(3S*)-1-{4-[4-(1,3-dioxolan-2-yl)piperidin-1-yl]phenyl}piperidin-3-yl]-4-methyl-1H-indole-3-carbonitrile [Absolute Stereochemistry not Yet Confirmed]

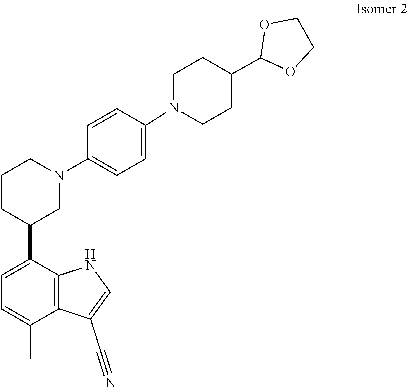

The title compounds were prepared using methodology described in intermediate 11c using 1-(4-bromophenyl)-4-(1,3-dioxolan-2-yl)piperidine and intermediate 78c to give a racemic mixture. The enantiomers were separated by preparative chiral-HPLC on a Column: CHIRAL ART Cellulose-SB, 2*25 cm, 5 μm; Mobile Phase A: Hex (0.5% 2M NH3-MeOH), Mobile Phase B: EtOH; Flow rate: 20 m/min; Gradient: 25% B in 10.5 mins to give, in order of elution, intermediate 82b (isomer 1, 70 mg, 15%, >99% ee) and intermediate 83a (isomer 2, 75 mg, 17%, >99% ee) as yellow solids.

### Example 82

### 7-[(3R*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-4-methyl-1H-indole-3-carbonitrile [Absolute Stereochemistry not Yet Confirmed]

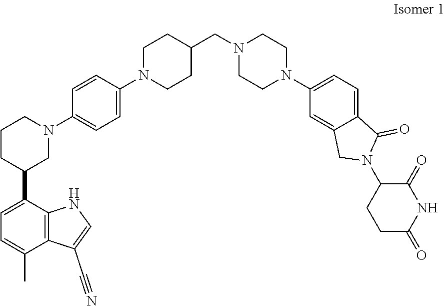 [SMILES: Cc1ccc([C@H]2CCCN(c3ccc(N4CCC(CN5CCN(c6ccc7c(c6)CN(C6CCC(=O)NC6=O)C7=O)CC5)CC4)cc3)C2)c2[nH]cc(C#N)c12]

The title compound was prepared using methodology described in example 24 using intermediate 82b and intermediate 1i and purified by flash C18-flash chromatography, elution gradient 0 to 90% MeCN in water (0.1% NHHCO₃) to give the title compound (35 mg, 32%) as a white solid. ¹H NMR δ 1.22 (3H, q), 1.6-1.64 (2H, m), 1.75-1.86 (4H, m), 1.9-1.98 (2H, m), 2.21 (2H, d), 2.36 (2H, dd), 2.50 (1H, s), 2.51-2.76 (8H, m), 2.83-2.96 (1H, m), 3.24-3.31 (4H, m), 3.36-3.43 (1H, m), 3.48 (2H, d), 3.55 (2H, t), 4.20 (1H, d), 4.33 (1H, d), 5.05 (1H, dd), 6.84 (4H, q), 6.93 (1H, d), 7.02-7.13 (3H, m), 7.52 (1H, d), 8.22 (1H, d), 10.96 (1H, s), 12.28 (1H, d); m/z: ES⁺ [M+H]⁺=739.4.

### Example 83

### 7-[(3S*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-4-methyl-1H-indole-3-carbonitrile [Absolute Stereochemistry not Yet Confirmed]

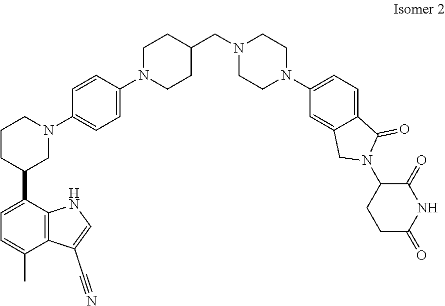 [SMILES: Cc1ccc([C@H]2CCCN(c3ccc(N4CCC(CN5CCN(c6ccc7c(c6)CN(C6CCC(=O)NC6=O)C7=O)CC5)CC4)cc3)C2)c2[nH]cc(C#N)c12]

The title compound was prepared using methodology described in example 24 using intermediate 83a and intermediate 1i and purified by flash C18-flash chromatography, elution gradient 0 to 90% MeCN in water (0.1% NH₄HCO₃) to give the title compound (11 mg, 9%) as a white solid. ¹H NMR δ 1.22 (3H, t), 1.62 (2H, s), 1.79 (4H, d), 1.94 (2H, s), 2.20 (2H, d), 2.32-2.4 (2H, m), 2.49-2.51 (2H, m), 2.54-2.65 (5H, m), 2.73-2.78 (2H, m), 2.90 (2H, t), 3.28 (4H, s), 3.34 (1H, s), 3.47 (2H, d), 3.52-3.59 (2H, m), 4.19 (1H, d), 4.32 (1H, d), 5.01-5.09 (1H, m), 6.77-6.89 (4H, m), 6.9-6.97 (1H, m), 7.02-7.13 (3H, m), 7.52 (1H, dd), 8.21 (1H, t), 10.95 (1H, d), 12.27 (1H, s); m/z: ES⁺ [M+H]⁺=739.4.

### Example 84

### Intermediate 84a

### 1-[1-Methyl-2-(piperidin-4-yl)-1H-indol-6-yl]-1,3-diazinane-2,4-dione

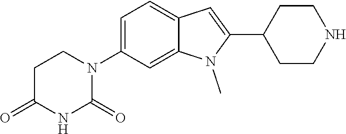 [SMILES: Cn1c(C2CCNCC2)cc2ccc(N3CCC(=O)NC3=O)cc21]

tert-Butyldimethylsilyl trifluoromethanesulfonate (81 μl, 0.35 mmol) was added dropwise to a slurry of tert-butyl 4-[6-(2,4-dioxo-1,3-diazinan-1-yl)-1-methyl-1H-indol-2-yl]piperidine-1-carboxylate (intermediate 13c) (100 mg, 0.23 mmol) in DCM (2 mL) at 0° C. The reaction was stirred to RT for 1 h and evaporated to dryness to give the title compound as a grey solid in quantitative yield which was used without further purification. m/z: ES⁺ [M+H]⁺=327.4.

### Example 84

### 4-Chloro-7-(4-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl)-1H-indole-3-carbonitrile

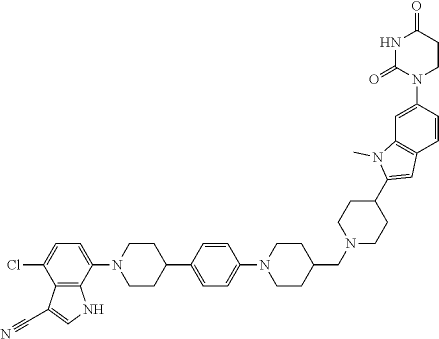 [SMILES: Cn1c(C2CCN(CC3CCN(c4ccc(C5CCN(c6ccc(Cl)c7c(C#N)c[nH]c67)CC5)cc4)CC3)CC2)cc2ccc(N3CCC(=O)NC3=O)cc21]

The title compound was prepared using methodology described in example 70 using 1-[1-methyl-2-(piperidin-4-yl)-1H-indol-6-yl]-1,3-diazinane-2,4-dione and intermediate if to give the title compound (6 mg, 5%) as a white solid. ¹H NMR δ 1.19-1.31 (3H, m), 1.62-1.74 (3H, m), 1.79-1.88 (4H, m), 1.89-2 (4H, m), 2.03-2.13 (2H, m), 2.23 (2H, d), 2.56-2.64 (2H, m), 2.7-2.85 (5H, m), 2.98 (2H, d), 3.45 (2H, d), 3.61-3.73 (5H, m), 3.79 (2H, t), 6.25 (1H, s), 6.85 (1H, d), 6.88-6.98 (3H, m), 7.1-7.21 (3H, m), 7.36 (1H, s), 7.43 (1H, d), 8.30 (1H, s), 10.27 (1H, s), 12.24 (1H, s); m/z: ES⁺ [M+H]⁺=757.8.

### Example 85

### Intermediate 85a

### tert-Butyl 4-{4-[4-(1,3-dioxolan-2-yl)piperidine-1-carbonyl]phenyl}piperidine-1-carboxylate

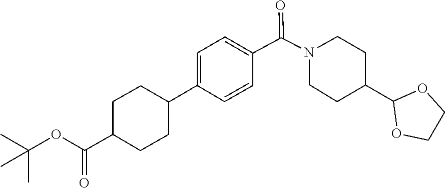 [SMILES: CC(C)(C)OC(=O)C1CCC(c2ccc(C(=O)N3CCC(C4OCCO4)CC3)cc2)CC1]

4-(1,3-Dioxolan-2-yl)piperidine (1.23 g, 7.86 mmol), HOBt (1.605 g, 10.48 mmol), 1-ethyl-3-carbodiimide hydrochloride (1.88 g, 9.82 mmol) and DIPEA (3.43 ml, 19.65 mmol) was added to 4-[1-(tert-butoxycarbonyl)piperidin-4-yl]benzoic acid (0.51 g, 6.55 mmol) in DMF (30 mL). The resulting mixture was stirred at RT for 16 h. The solvent was removed under reduced pressure to afford crude product which was purified by flash silica chromatography, elution gradient 0 to 30% EtOAc in Et₂O to give the title compound (0.51 g, 17.5%) as a white solid. ¹H NMR δ 1.23 (2H, d), 1.42 (9H, s), 1.44-1.58 (1H, m), 1.70 (2H, s), 1.77 (3H, d), 2.70 (2H, d), 2.77 (1H, s), 2.82 (2H, s), 3.17 (4H, d), 3.72-3.92 (3H, m), 4.10 (2H, q), 4.61 (1H, d), 7.30 (4H, s); m/z: ES⁺ [M+H]⁺=277.2.

### Intermediate 85b

### [4-(1,3-Dioxolan-2-yl)piperidin-1-yl][4-(piperidin-4-yl)phenyl]methanone

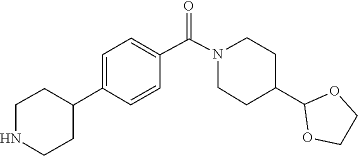 [SMILES: O=C(c1ccc(C2CCNCC2)cc1)N1CCC(C2OCCO2)CC1]

The title compound was prepared using methodology described in intermediate 27b using [4-(1,3-dioxolan-2-yl)piperidin-1-yl][4-(piperidin-4-yl)phenyl]methanone to give the title compound which was used without further purification. no analytical data

### Intermediate 85c

### 4-Chloro-7-(4-{4-[4-(1,3-dioxolan-2-yl)piperidine-1-carbonyl]phenyl}piperidin-1-yl)-1H-indole-3-carbonitrile

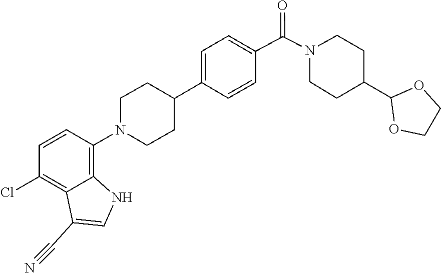 [SMILES: N#Cc1c[nH]c2c(N3CCC(c4ccc(C(=O)N5CCC(C6OCCO6)CC5)cc4)CC3)ccc(Cl)c12]

The title compound was prepared using methodology described in intermediate 11c using [4-(1,3-dioxolan-2-yl)piperidin-1-yl][4-(piperidin-4-yl)phenyl]methanone and intermediate 1b to give the title compound (271 mg, 45%) as a white solid. ¹H NMR δ −0.12 (8H, s), 1.03 (1H, s), 2.71 (5H, d), 3.15 (4H, s), 3.59-4.05 (4H, m), 4.50 (1H, s), 5.64 (1H, s), 6.40 (1H, s), 6.75 (1H, s), 7.05 (1H, d), 7.24 (2H, s), 8.22 (1H, d), 12.17 (1H, s); m/z: ES⁺ [M+H]⁺=681.5.

### Example 85

### 4-Chloro-7-(4-{4-[4-({4-[2-(2,6-dioxopiperidin-3-yl)-1-oxo-2,3-dihydro-1H-isoindol-5-yl]piperazin-1-yl}methyl)piperidine-1-carbonyl]phenyl}piperidin-1-yl)-1H-indole-3-carbonitrile

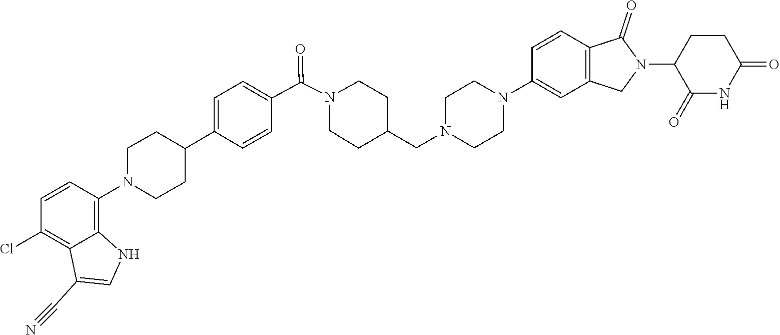 [SMILES: N#Cc1c[nH]c2c(N3CCC(c4ccc(C(=O)N5CCC(CN6CCN(c7ccc8c(c7)CN(C7CCC(=O)NC7=O)C8=O)CC6)CC5)cc4)CC3)ccc(Cl)c12]

The title compound was prepared using methodology described in example 24 using 4-chloro-7-(4-{4-[4-(1,3-dioxolan-2-yl)piperidine-1-carbonyl]phenyl}piperidin-1-yl)-1H-indole-3-carbonitrile and intermediate 1i followed by purification by flash C18-flash chromatography, elution gradient 0 to 100% MeCN in water (0.1% NH₄HCO₃) followed by purification by preparative TLC DCM:MeOH (14:1) to give the title compound (93 mg, 61%) as a white solid. ¹H NMR δ 1.01-1.11 (2H, m), 1.6-2.1 (8H, m), 2.18 (2H, d), 2.34 (1H, dd), 2.42-2.48 (4H, m), 2.5-2.62 (1H, m), 2.65-3.11 (6H, m), 3.21-3.29 (4H, m), 3.38-3.65 (3H, m), 4.12-4.36 (2H, m), 4.44 (1H, s), 5.03 (1H, dd), 6.84 (1H, d), 6.98-7.07 (2H, m), 7.15 (1H, d), 7.31-7.37 (4H, m), 7.50 (1H, d), 8.32 (1H, s), 10.95 (1H, s), 12.24 (1H, s); m/z: ES⁺ [M+H]⁺=787.4.

### Example 86

### Intermediate 86a

### Benzyl 4-[4-(1,4-dioxa-8-azaspiro[4.5]decan-8-yl)phenyl]piperidine-1-carboxylate

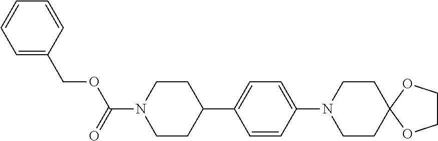 [SMILES: O=C(OCc1ccccc1)N1CCC(c2ccc(N3CCC4(CC3)OCCO4)cc2)CC1]

The title compound was prepared using methodology described in intermediate 9g using 1,4-dioxa-8-azaspiro[4.5]decane and intermediate 1c to give the title compound (5.5 g, 94%) as a white solid. ¹H NMR δ 1.59 (8H, dt), 2.52-2.66 (1H, m), 3.15-3.26 (4H, m), 4.01-4.09 (8H, m), 5.10 (2H, s), 6.87 (2H, d), 7.06 (2H, d), 7.22-7.46 (5H, m); m/z: ES⁺ [M+H]⁺=437.3.

### Intermediate 86b

### 8-[4-(Piperidin-4-yl)phenyl]-1,4-dioxa-8-azaspiro[4.5]decane

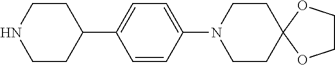 [SMILES: c1cc(N2CCC3(CC2)OCCO3)ccc1C1CCNCC1]

The title compound was prepared using methodology described in intermediate 1b using benzyl 4-[4-(1,4-dioxa-8-azaspiro[4.5]decan-8-yl)phenyl]piperidine-1-carboxylate to give the title compound (300 mg, 71%) as a white solid. ¹H NMR δ 1.34 (2H, dd), 1.44-1.63 (6H, m), 2.37 (4H, s), 2.45 (1H, d), 2.88 (2H, d), 3.07 (2H, t), 3.78 (4H, s), 6.7-6.79 (2H, m), 6.92 (2H, d); m/z: ES⁺ [M+H]⁺=303.2.

### Intermediate 86c

### 4-Chloro-7-{4-[4-(1,4-dioxa-8-azaspiro[4.5]decan-8-yl)phenyl]piperidin-1-yl}-1H-indole-3-carbonitrile

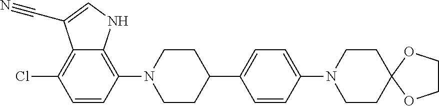 [SMILES: N#Cc1c[nH]c2c(N3CCC(c4ccc(N5CCC6(CC5)OCCO6)cc4)CC3)ccc(Cl)c12]

The title compound was prepared using methodology described in intermediate 23a using 8-[4-(piperidin-4-yl)phenyl]-1,4-dioxa-8-azaspiro[4.5]decane and intermediate 1b to give the title compound (211 mg, 45%) as a white solid. ¹H NMR δ 1.69 (2H, s), 1.80 (2H, s), 1.84 (2H, s), 1.91 (2H, d), 1.99 (2H, d), 3.19 (2H, s), 3.21 (2H, s), 3.23 (1H, s), 3.89 (4H, s), 6.85 (1H, s), 6.91 (2H, d), 7.10 (2H, s), 7.13 (2H, s), 7.16 (1H, s), 8.31 (1H, d); m/z: ES⁺ [M+H]⁺=477.2.

### Example 86

### 4-Chloro-7-{4-[4-(4-{4-[2-(2,6-dioxopiperidin-3-yl)-4-methoxy-3-oxo-2,3-dihydro-1H-pyrrolo[3,4-c]pyridin-6-yl]piperazin-1-yl}piperidin-1-yl)phenyl]piperidin-1-yl}-1H-indole-3-carbonitrile

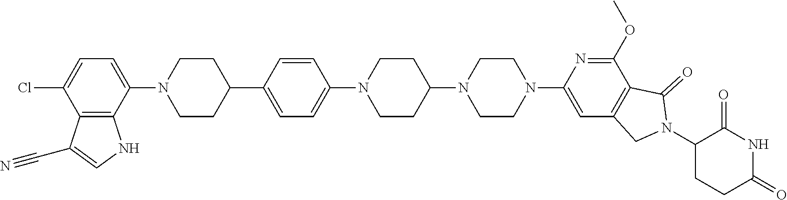 [SMILES: COc1nc(N2CCN(C3CCN(c4ccc(C5CCN(c6ccc(Cl)c7c(C#N)c[nH]c67)CC5)cc4)CC3)CC2)cc2c1C(=O)N(C1CCC(=O)NC1=O)C2]

The title compound was prepared using methodology described in example 24 using 4-chloro-7-{4-[4-(1,4-dioxa-8-azaspiro[4.5]decan-8-yl)phenyl]piperidin-1-yl}-1H-indole-3-carbonitrile and intermediate 30e to give the title compound (35 mg, 17%) as a white solid. ¹H NMR δ 1.51-1.58 (2H, m), 1.76-2.04 (7H, m), 2.26-2.4 (2H, m), 2.59-2.64 (8H, m), 2.78 (2H, t), 2.84-2.96 (1H, m), 3.38-3.45 (2H, m), 3.59-3.63 (4H, m), 3.67-3.74 (2H, m), 3.89 (3H, s), 4.13 (1H, d), 4.28 (1H, d), 4.93-5.01 (1H, m), 6.49 (1H, s), 6.84-6.96 (3H, m), 7.11-7.2 (3H, m), 8.34 (1H, s), 10.94 (1H, s), 12.29 (1H, s); m/z: ES⁺ [M+H]⁺=776.6.

### Example 87

### Intermediate 87a

### Benzyl 4-(4-{4-[(1,3-dioxolan-2-yl)methyl]piperidin-1-yl}phenyl)piperidine-1-carboxylate

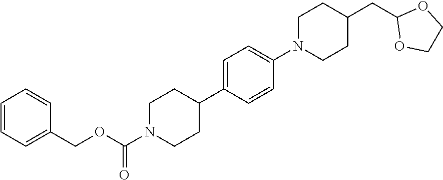 [SMILES: O=C(OCc1ccccc1)N1CCC(c2ccc(N3CCC(CC4OCCO4)CC3)cc2)CC1]

The title compound was prepared using methodology described in intermediate 9g using 4-[(1,3-dioxolan-2-yl)methyl]piperidine and intermediate 1c to give the title compound (1.6 g, 81%) as a yellow solid. ¹H NMR S 1.21-1.34 (2H, m), 1.38-1.6 (5H, m), 1.66-1.82 (4H, m), 2.52-2.65 (3H, m), 2.83-2.96 (2H, m), 3.53-3.62 (2H, m), 3.69-3.82 (2H, m), 3.82-3.94 (2H, m), 4.08-4.16 (2H, m), 4.87 (1H, t), 5.09 (2H, s), 6.84 (2H, d), 7.05 (2H, d), 7.28-7.43 (5H, m); m/z: ES⁺ [M+H]⁺=465.3.

### Intermediate 87b

### 4-[(1,3-Dioxolan-2-yl)methyl]-1-[4-(piperidin-4-yl)phenyl]piperidine

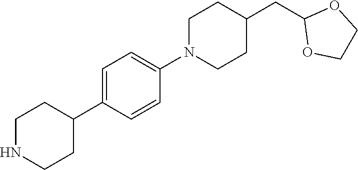 [SMILES: c1cc(N2CCC(CC3OCCO3)CC2)ccc1C1CCNCC1]

The title compound was prepared using methodology described in intermediate 1b using benzyl 4-(4-{4-[(1,3-dioxolan-2-yl)methyl]piperidin-1-yl}phenyl)piperidine-1-carboxylate to give the title compound (410 mg, 72%) as a white solid. ¹H NMR δ 1.22-1.33 (2H, m), 1.38-1.46 (1H, m), 1.42-1.55 (1H, m), 1.51-1.66 (5H, m), 1.78 (2H, d), 2.37-2.48 (1H, m), 2.52-2.6 (4H, m), 2.94-3.03 (2H, m), 3.52-3.61 (2H, m), 3.69-3.82 (2H, m), 3.82-3.94 (2H, m), 4.87 (1H, t), 6.8-6.9 (2H, m), 7-7.07 (2H, m); m/z: ES⁺ [M+H]⁺=331.2.

### Intermediate 87c

### 4-Chloro-7-[4-(4-{4-[(1,3-dioxolan-2-yl)methyl]piperidin-1-yl}phenyl)piperidin-1-yl]-1H-indole-3-carbonitrile

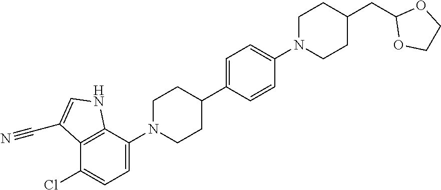 [SMILES: N#Cc1c[nH]c2c(N3CCC(c4ccc(N5CCC(CC6OCCO6)CC5)cc4)CC3)ccc(Cl)c12]

The title compound was prepared using methodology described in intermediate 23a using 4-[(1,3-dioxolan-2-yl)methyl]-1-[4-(piperidin-4-yl)phenyl]piperidine and intermediate 1b to give the title compound (120 mg, 19%) as a white solid. ¹H NMR δ 1.24-1.36 (2H, m), 1.51-1.57 (3H, m), 1.75-1.87 (4H, m), 1.96 (2H, q), 2.56-2.63 (2H, m), 2.77 (2H, t), 3.17 (1H, d), 3.41 (2H, d), 3.60 (2H, d), 3.72-3.86 (2H, m), 3.86-3.91 (2H, m), 4.88 (1H, t), 6.82-6.93 (3H, m), 7.08-7.2 (3H, m), 8.33 (1H, s), 12.28 (1H, s); m/z: ES⁺ [M+H]⁺=505.3.

### Example 87

### 4-Chloro-7-(4-{4-[4-(2-{4-[2-(2,6-dioxopiperidin-3-yl)-4-methoxy-3-oxo-2,3-dihydro-1H-pyrrolo[3,4-c]pyridin-6-yl]piperazin-1-yl}ethyl)piperidin-1-yl]phenyl}piperidin-1-yl)-1H-indole-3-carbonitrile

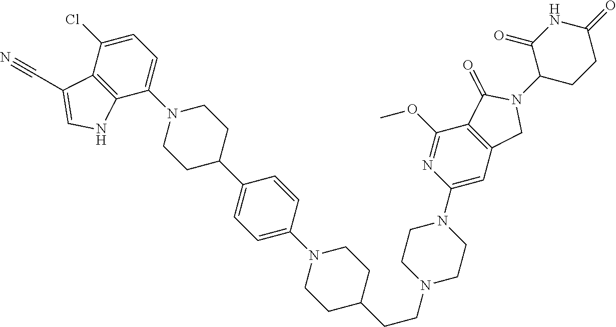 [SMILES: COc1nc(N2CCN(CCC3CCN(c4ccc(C5CCN(c6ccc(Cl)c7c(C#N)c[nH]c67)CC5)cc4)CC3)CC2)cc2c1C(=O)N(C1CCC(=O)NC1=O)C2]

The title compound was prepared using methodology described in example 24 using 4-chloro-7-[4-(4-{4-[(1,3-dioxolan-2-yl)methyl]piperidin-1-yl}phenyl)piperidin-1-yl]-1H-indole-3-carbonitrile and intermediate 30e to give the title compound (51 mg, 29%) as a white solid. ¹H NMR δ 1.21-1.31 (2H, m), 1.42-1.48 (3H, m), 1.7-2.05 (7H, m), 2.25-2.47 (7H, m), 2.53-2.67 (4H, m), 2.71-3.02 (3H, m), 3.37-3.47 (2H, m), 3.58-3.64 (6H, m), 3.89 (3H, s), 4.07-4.33 (2H, m), 4.97 (1H, dd), 6.49 (1H, s), 6.82-6.95 (3H, m), 7.09-7.21 (3H, m), 8.33 (1H, s), 10.93 (1H, s), 12.28 (1H, s); m/z: ES⁺ [M+H]⁺=804.4.

### Example 88

### Intermediate 88a

### Benzyl 4-(3-oxopropyl)piperidine-1-carboxylate

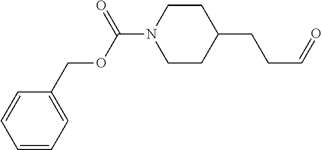 [SMILES: O=CCCC1CCN(C(=O)OCc2ccccc2)CC1]

Pyridinium chlorochromate (4.66 g, 21.63 mmol) was added to benzyl 4-(3-hydroxypropyl)piperidine-1-carboxylate (4.0 g, 14.42 mmol) in DCM (75 mL) at RT for 2 h. The solvent was evaporated to afford crude product. The crude product was purified by flash silica chromatography, elution gradient 0 to 30% EtOAc in Et₂O to give the title compound (2.8 g, 70.5%) as a colourless oil. ¹H NMR δ 0.87-1.06 (2H, m), 1.28-1.52 (3H, m), 1.57-1.67 (2H, m), 2.39-2.5 (2H, m), 2.73 (2H, s), 3.92-4.04 (2H, m), 5.04 (2H, s), 7.24-7.42 (5H, m), 9.66 (1H, t); m/z: ES⁺ [M+H]⁺=276.2.

### Intermediate 88b

### Benzyl 4-[2-(1,3-dioxolan-2-yl)ethyl]piperidine-1-carboxylate

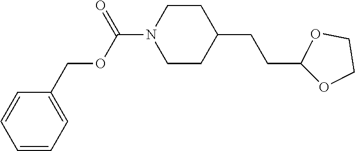 [SMILES: O=C(OCc1ccccc1)N1CCC(CCC2OCCO2)CC1]

p-Toluenesulfonic acid (0.093 g, 0.49 mmol) was added to benzyl 4-(3-oxopropyl)piperidine-1-carboxylate (2.7 g, 9.81 mmol) in glycol (50 mL) at RT under nitrogen and stirred at 100° C. for 16 h. The reaction mixture was diluted with EtOAc (500 mL), washed with water (100 mL), NaHCO₃ solution, NaCl solution and dried over Na₂SO₄. The organic layer was filtered and evaporated to afford crude product. The crude product was purified by flash silica chromatography, elution gradient 0 to 30% EtOAc in Et₂O to give the title compound (1.2 g, 38.3%) as a colourless liquid; m/z: ES⁺ [M+H]⁺=276.2.

### Intermediate 88c

### 4-[2-(1,3-Dioxolan-2-yl)ethyl]piperidine

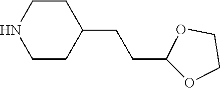 [SMILES: C1CC(CCC2OCCO2)CCN1]

The title compound was prepared using methodology described in intermediate 1b using benzyl 4-[2-(1,3-dioxolan-2-yl)ethyl]piperidine-1-carboxylate to give the title compound (811 mg, 88%) as a white solid. ¹H NMR δ 1.41 (4H, s), 1.44 (5H, s), 2.26 (1H, s), 2.74 (2H, s), 2.78 (2H, s), 3.62 (2H, s), 3.74 (2H, s), 4.62 (1H, s); m/z: ES⁺ [M+H]⁺=186.3.

### Intermediate 88d

### Benzyl 4-(4-{4-[2-(1,3-dioxolan-2-yl)ethyl]piperidin-1-yl}phenyl)piperidine-1-carboxylate

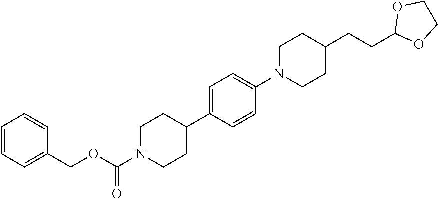 [SMILES: O=C(OCc1ccccc1)N1CCC(c2ccc(N3CCC(CCC4OCCO4)CC3)cc2)CC1]

The title compound was prepared using methodology described in intermediate 11a using 4-[2-(1,3-dioxolan-2-yl)ethyl]piperidine and intermediate 1c to give the title compound (547 mg, 39%) as a white solid; m/z: ES⁺ [M+H]⁺=237.0.

### Intermediate 88e

### 4-[2-(1,3-Dioxolan-2-yl)ethyl]-1-[4-(piperidin-4-yl)phenyl]piperidine

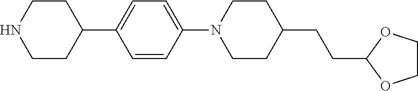 [SMILES: c1cc(N2CCC(CCC3OCCO3)CC2)ccc1C1CCNCC1]

The title compound was prepared using methodology described in intermediate 1b using benzyl 4-(4-{4-[2-(1,3-dioxolan-2-yl)ethyl]piperidin-1-yl}phenyl)piperidine-1-carboxylate to give the title compound (350 mg, 97%) as a white solid. ¹H NMR δ 1.25 (4H, dd), 1.34 (5H, s), 1.58-1.74 (4H, m), 2.17 (1H, s), 2.55 (8H, d), 2.98 (1H, d), 3.67-3.77 (2H, m), 3.81-3.91 (2H, m), 4.73 (1H, s), 6.81 (1H, s), 6.84 (1H, d), 7.00 (1H, s), 7.03 (1H, s); m/z: ES⁺ [M+H]⁺=324.3.

### Intermediate 88f

### 4-Chloro-7-[4-(4-{4-[2-(1,3-dioxolan-2-yl)ethyl]piperidin-1-yl}phenyl)piperidin-1-yl]-1H-indole-3-carbonitrile

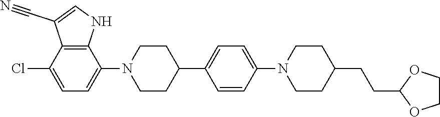 [SMILES: N#Cc1c[nH]c2c(N3CCC(c4ccc(N5CCC(CCC6OCCO6)CC5)cc4)CC3)ccc(Cl)c12]

The title compound was prepared using methodology described in intermediate 23a using 4-[2-(1,3-dioxolan-2-yl)ethyl]-1-[4-(piperidin-4-yl)phenyl]piperidine and intermediate 1b to give the title compound (200 mg, 33%) as a white solid; m/z: ES⁺ [M+H]⁺=519.4. no nmr data

### Example 88

### 4-Chloro-7-(4-{4-[4-(3-{4-[2-(2,6-dioxopiperidin-3-yl)-4-methoxy-3-oxo-2,3-dihydro-1H-pyrrolo[3,4-c]pyridin-6-yl]piperazin-1-yl}propyl)piperidin-1-yl]phenyl}piperidin-1-yl)-1H-indole-3-carbonitrile

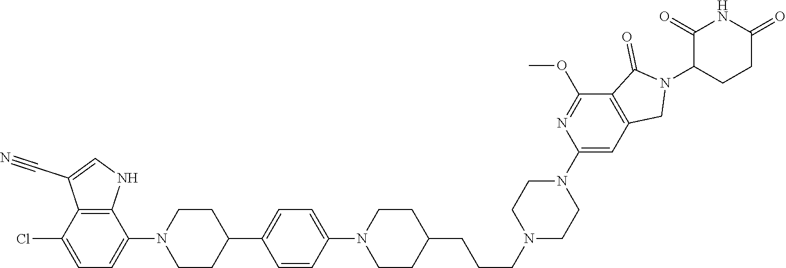 [SMILES: COc1nc(N2CCN(CCCC3CCN(c4ccc(C5CCN(c6ccc(Cl)c7c(C#N)c[nH]c67)CC5)cc4)CC3)CC2)cc2c1C(=O)N(C1CCC(=O)NC1=O)C2]

The title compound was prepared using methodology described in example 78 using 4-chloro-7-[4-(4-{4-[2-(1,3-dioxolan-2-yl)ethyl]piperidin-1-yl}phenyl)piperidin-1-yl]-1H-indole-3-carbonitrile and intermediate 30e to give the title compound (49 mg, 26%) as a white solid. ¹H NMR δ 1.18-1.29 (4H, m), 1.35 (1H, s), 1.51 (2H, s), 1.75 (2H, d), 1.83 (2H, d), 1.89-2.03 (3H, m), 2.27-2.35 (3H, m), 2.45 (4H, s), 2.56 (4H, d), 2.76 (2H, t), 2.89 (1H, t), 3.41 (2H, d), 3.62 (6H, d), 3.89 (3H, s), 4.13 (1H, d), 4.27 (1H, d), 4.97 (1H, dd), 6.48 (1H, s), 6.87 (3H, dd), 7.14 (3H, dd), 8.33 (1H, s), 10.94 (1H, s), 12.29 (1H, s); m/z: ES⁺ [M+H]⁺=818.5.

### Example 89

### Intermediate 89a

### tert-Butyl 4-(5-bromo-1H-indol-1-yl)piperidine-1-carboxylate

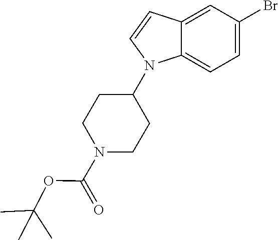

tert-Butyl 4-[(methanesulfonyl)oxy]piperidine-1-carboxylate (534 g, 1912.81 mmol) and potassium tert-butoxide (258 g, 2295.37 mmol) were added portion wise to 5-bromo-1H-indole (150 g, 765.12 mmol) in heptane (2400 mL) and stirred at 80° C. for 3 h. The reaction mixture was cooled to RT and was diluted with MTBE. The solid was filtered and the filtrate was evaporated to dryness. The residue was redissolved in EtOAc, washed with water, dried over Na₂SO₄, filtered and evaporated to afford crude product. The crude product was purified by flash silica chromatography, elution gradient 0 to 30% EtOAc in Et₂O to give a colourless foam which was crystallised from MTBE:Et₂O (1:5) to give the title compound (85 g, 29.3%) as a white solid. ¹H NMR (CDCl₃) δ 1.36-1.42 (9H, s), 1.69-1.89 (2H, m), 1.92-2.02 (2H, d), 2.73-2.89 (2H, m), 4.14-4.29 (3H, m), 6.33-6.40 (1H, m), 7.04-7.12 (1H, d), 7.09-7.23 (2H, m), 7.60-7.69 (1H, m); m/z: ES⁺ [M-tBu]⁺=323.0.

### Intermediate 89b

### tert-Butyl 4-[5-(2,4-dioxo-1,3-diazinan-1-yl)-1H-indol-1-yl]piperidine-1-carboxylate

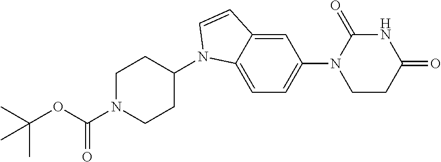 [SMILES: CC(C)(C)OC(=O)N1CCC(n2ccc3cc(N4CCC(=O)NC4=O)ccc32)CC1]

[Pd(cinnamyl)Cl]₂ (0.913 g, 1.65 mmol) was added to tert-butyl 4-(5-bromo-1H-indol-1-yl)piperidine-1-carboxylate (25 g, 65.91 mmol), 1,3-diazinane-2,4-dione (15.04 g, 131.82 mmol), tert-butylBrettPhos (1.59 g, 3.30 mmol) and Cs₂CO₃ (43 g, 131.82 mmol) in 1,4-dioxane (750 mL) at RT under nitrogen. The resulting mixture was stirred at 90° C. for 16 h. The reaction mixture was diluted with EtOAc (2 L) and water (2 L). The organic layer was dried over Na₂SO₄, filtered and evaporated to afford crude product. The crude product was purified by crystallisation from MTBE to give the title compound (44.0 g, 57.1%) as a white solid. ¹H NMR (CDCl₃) δ 1.49-1.54 (9H, s), 1.85-2.00 (2H, qd), 2.04-2.13 (2H, m), 2.84-3.00 (4H, m), 3.88-3.95 (2H, m), 4.32-4.44 (3H, m), 6.52-6.58 (1H, m), 7.12-7.19 (1H, m), 7.22-7.27 (1H, d), 7.38-7.46 (2H, m), 7.52-7.57 (1H, d); m/z: ES⁺ [M+H]⁺=413.0.

### Intermediate 89c

### 1-[1-(Piperidin-4-yl)-1H-indol-5-yl]-1,3-diazinane-2,4-dione

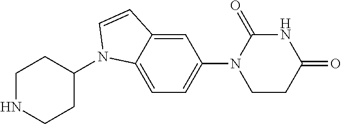 [SMILES: O=C1CCN(c2ccc3c(ccn3C3CCNCC3)c2)C(=O)N1]

The title compound was prepared using methodology described in intermediate 5f using tert-butyl 4-[5-(2,4-dioxo-1,3-diazinan-1-yl)-1H-indol-1-yl]piperidine-1-carboxylate to give the title compound as a tosylate salt (73 g, 82%) as a white solid. ¹H NMR δ 2.05-2.20 (4H, m), 2.27-2.32 (3H, d), 2.69-2.77 (2H, m), 3.15-3.20 (2H, s), 3.44-3.51 (2H, d), 3.73-3.81 (2H, m), 4.71-4.75 (1H, s), 6.48-6.54 (1H, m), 7.08-7.16 (3H, m), 7.39-7.47 (1H, m), 7.47-7.53 (3H, m), 7.53-7.63 (1H, m), 8.37-8.42 (1H, s), 8.61-8.65 (1H, s), 10.24-10.29 (1H, d); m/z: ES⁺ [M+H]⁺=313.0.

### Example 89

### 7-(4-{4-[4-({4-[5-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile

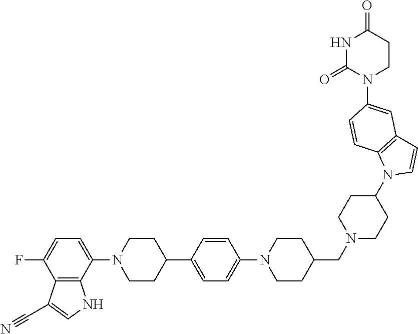 [SMILES: N#Cc1c[nH]c2c(N3CCC(c4ccc(N5CCC(CN6CCC(n7ccc8cc(N9CCC(=O)NC9=O)ccc87)CC6)CC5)cc4)CC3)ccc(F)c12]

The title compound was prepared using methodology described in example 23 using 1-[1-(piperidin-4-yl)-1H-indol-5-yl]-1,3-diazinane-2,4-dione and intermediate 2c to give the title compound in the form of a formate salt (46 mg, 56%) as a white solid. ¹H NMR (90° C.) δ 1.25-1.37 (2H, m), 1.66-1.77 (1H, m), 1.83-1.92 (4H, m), 1.94-2.09 (7H, m), 2.2-2.35 (5H, m), 2.69-2.76 (4H, m), 2.84 (2H, t), 3.03 (2H, d), 3.40 (2H, d), 3.64 (2H, d), 3.79 (2H, t), 4.29-4.43 (1H, m), 6.47 (1H, d), 6.82-6.89 (2H, m), 6.91 (3H, d), 7.07-7.13 (1H, m), 7.16 (2H, d), 7.45-7.55 (3H, m), 8.11 (1H, s), 9.85 (1H, s); m/z: ES⁺ [M+H]⁺=727.8.

### Example 90

### 4-Chloro-7-(4-{4-[4-({4-[5-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile

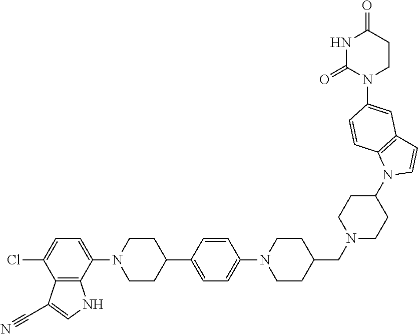 [SMILES: N#Cc1c[nH]c2c(N3CCC(c4ccc(N5CCC(CN6CCC(n7ccc8cc(N9CCC(=O)NC9=O)ccc87)CC6)CC5)cc4)CC3)ccc(Cl)c12]

The title compound was prepared using methodology described in example 23 using intermediate 89c and intermediate if to give the title compound in the form of a formate salt (34 mg, 53%) as a white solid. ¹H NMR δ 1.17-1.33 (2H, m), 1.59-1.74 (1H, m), 1.77-1.89 (4H, m), 1.89-2.09 (6H, m), 2.18 (2H, t), 2.26 (2H, d), 2.6-2.83 (7H, m), 3.01 (2H, d), 3.39-3.46 (2H, m), 3.61-3.71 (2H, m), 3.77 (2H, t), 4.31-4.44 (1H, m), 6.47 (1H, d), 6.79-6.99 (3H, m), 7.04-7.23 (4H, m), 7.47 (1H, d), 7.5-7.63 (2H, m), 8.32 (1H, s), 10.25 (1H, s), 12.26 (1H, s); m/z: ES⁺ [M+H]⁺=743.9.

### Example 91

### Intermediate 91a

### tert-Butyl 5-(4-chloro-3-cyano-1H-indol-7-yl)-3,6-dihydropyridine-1(2H)-carboxylate

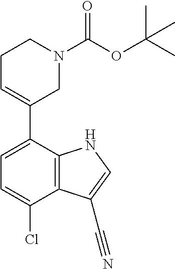 [SMILES: CC(C)(C)OC(=O)N1CCC=C(c2ccc(Cl)c3c(C#N)c[nH]c23)C1]

The title compound was prepared using methodology described in intermediate 63b using intermediate 1b and tert-butyl 5-(4,4,5,5-tetramethyl-1,3,2-dioxaborolan-2-yl)-3,4-dihydropyridine-1(2H)-carboxylate to give the title compound (2.5 g, 59%) as a yellow solid. ¹H NMR (CDCl₃) δ 1.15-1.54 (9H, m), 2.29 (2H, d), 3.57 (2H, s), 4.05 (2H, s), 6.90 (1H, d), 7.06 (1H, s), 7.16 (1H, d), 7.73 (1H, s), 11.02 (1H, s); m/z: ES⁻[M−H]⁻=356.1.

### Intermediate 91b

### tert-Butyl 3-(4-chloro-3-cyano-1H-indol-7-yl)piperidine-1-carboxylate

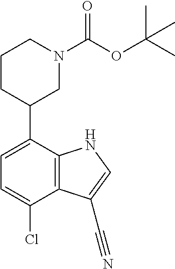 [SMILES: CC(C)(C)OC(=O)N1CCCC(c2ccc(Cl)c3c(C#N)c[nH]c23)C1]

Royer® Rhodium Catalyst Beads (1.26 g, 0.61 mmol) was added to tert-butyl 5-(4-chloro-3-cyano-1H-indol-7-yl)-3,6-dihydropyridine-1(2H)-carboxylate (2.2 g, 6.15 mmol) in MeOH (10 mL) and THF (10 mL) under an atmosphere of hydrogen. The resulting mixture was stirred at 80° C. for 16 h. The catalyst was filtered off through a pad of Celite® and evaporated to dryness to afford crude product. The crude product was purified by flash silica chromatography, elution gradient 0 to 80% EtOAc in Et₂O to give the title compound (1.4 g, 63.3%) as a yellow solid. ¹H NMR δ 1.41 (9H, d), 1.60 (1H, t), 1.71 (1H, s), 1.94 (1H, d), 2.85 (1H, s), 3.18 (2H, d), 3.34 (2H, s), 4.00 (2H, d), 4.07-4.17 (1H, m), 7.16 (1H, d), 7.19-7.31 (1H, m), 8.43 (1H, d), 12.56-12.63 (1H, m); m/z: ES⁺ [M+H]⁺=360.0.

### Intermediate 91c

### 4-Chloro-7-(piperidin-3-yl)-1H-indole-3-carbonitrile

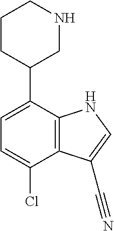 [SMILES: N#Cc1c[nH]c2c(C3CCCNC3)ccc(Cl)c12]

The title compound was prepared using methodology described in intermediate 78c using tert-butyl 3-(4-chloro-3-cyano-1H-indol-7-yl)piperidine-1-carboxylate (780 mg, 77%) as a white solid. ¹H NMR δ 1.65-1.87 (3H, m), 1.93 (1H, s), 2.70-2.87 (2H, m), 3.10-3.28 (2H, m), 7.04-7.17 (1H, m), 7.23 (1H, d), 8.44 (2H, d); m/z: ES⁺ [M+H]⁺=260.0.

### Intermediate 91d

### 1-(4-Bromophenyl)-4-(dibutoxymethyl)piperidine

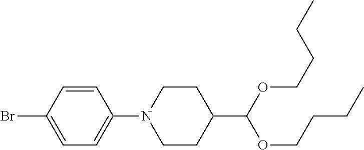 [SMILES: CCCCOC(OCCCC)C1CCN(c2ccc(Br)cc2)CC1]

The title compound was prepared using methodology described in intermediate 82a using bromo-4-iodobenzene and 4-(dibutoxymethyl)piperidine to give the title compound (1.4 g, 49%) as a yellow solid. ¹H NMR δ 0.89 (3H, t), 1.20-1.36 (2H, m), 1.32-1.58 (5H, m), 1.65-1.76 (2H, m), 2.47-2.55 (1H, m), 2.56-2.67 (1H, m), 3.32-3.45 (1H, m), 3.49-3.62 (1H, m), 3.68 (1H, d), 4.14-4.23 (1H, m), 6.83-6.96 (1H, m), 7.25-7.37 (1H, m); m/z: ES⁺ [M+H]⁺=398.2.

### Intermediate 91e

### 4-Chloro-7-[(3R*)-1-{4-[4-(dibutoxymethyl)piperidin-1-yl]phenyl}piperidin-3-yl]-1H-indole-3-carbonitrile [Absolute Stereochemistry not Yet Confirmed]

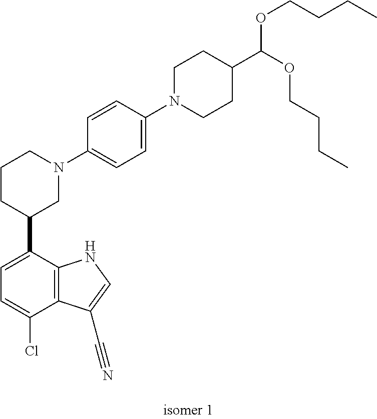

### and

### Intermediate 92a

### 4-Chloro-7-[(3S*)-1-{4-[4-(dibutoxymethyl)piperidin-1-yl]phenyl}piperidin-3-yl]-1H-indole-3-carbonitrile [Absolute Stereochemistry not Yet Confirmed]

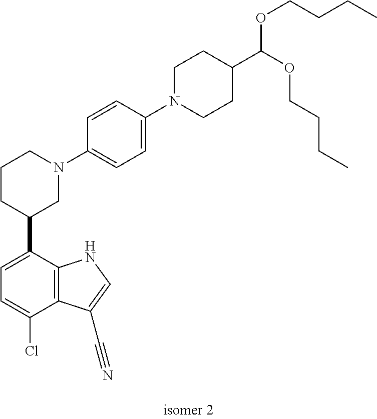

The title compounds were prepared using methodology described in intermediate 78e and intermediate 79a using 4-chloro-7-(piperidin-3-yl)-1H-indole-3-carbonitrile and 1-(4-bromophenyl)-4-(dibutoxymethyl)piperidine to give a racemic mixture. The enantiomers were separated by preparative chiral-HPLC on a Column: CHIRAL PAK A1-3, 4.6*250 cm, 3 μm; Mobile Phase A: Hex (0.1% DEA), Mobile Phase B: EtOH; Flow rate: 1 mL/min; Gradient: 20% B in 10.5 mins to give, in order of elution, intermediate 91e (isomer 1, 80 mg, 4.5%, >99% ee, m/z: ES⁺, [M+H]⁺=577.0.) and intermediate 92a (isomer 2, 85 mg, 5%, >99% ee, m/z: ES⁺, [M+H]⁺=577.0.) as yellow solids.

### Example 91

### 4-Chloro-7-[(3R*)-1-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-3-yl]-1H-indole-3-carbonitrile [Absolute Stereochemistry not Yet Confirmed]

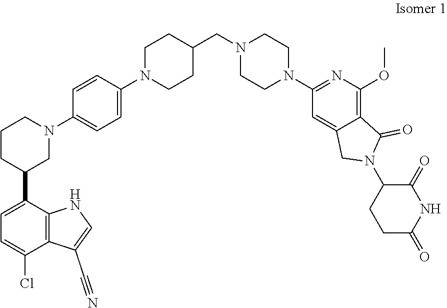 [SMILES: COc1nc(N2CCN(CC3CCN(c4ccc(N5CCC[C@H](c6ccc(Cl)c7c(C#N)c[nH]c67)C5)cc4)CC3)CC2)cc2c1C(=O)N(C1CCC(=O)NC1=O)C2]

The title compound was prepared using methodology described in example 24 using intermediate 91e and intermediate 30e and purified by flash C18-flash chromatography, elution gradient 0 to 90% MeCN in water (0.1% NH₄HCO₃) to give the title compound (24 mg, 44%) as a pale yellow solid. ¹H NMR δ 1.22 (3H, d), 1.64 (2H, s), 1.72-2.02 (6H, m), 2.21 (2H, d), 2.25-2.37 (1H, m), 2.44 (3H, s), 2.62 (2H, d), 2.76-2.95 (4H, m), 3.42-3.70 (9H, m), 3.89 (3H, s), 4.11-4.31 (2H, m), 4.91-5.03 (1H, m), 6.49 (1H, s), 6.78-6.93 (4H, m), 7.24 (2H, s), 8.39 (1H, s), 10.93 (1H, s), 12.67 (1H, s); m/z: ES⁺ [M+H]⁺=790.0.

### Example 92

### 4-Chloro-7-[(3S*)-1-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-3-yl]-1H-indole-3-carbonitrile [Absolute Stereochemistry not Yet Confirmed]

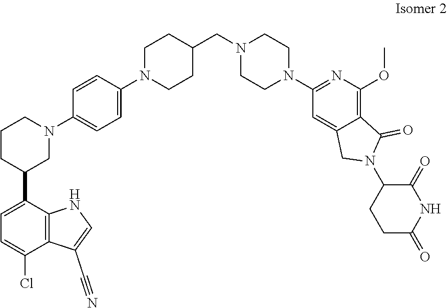 [SMILES: COc1nc(N2CCN(CC3CCN(c4ccc(N5CCC[C@H](c6ccc(Cl)c7c(C#N)c[nH]c67)C5)cc4)CC3)CC2)cc2c1C(=O)N(C1CCC(=O)NC1=O)C2]

The title compound was prepared using methodology described in example 24 using intermediate 92a and intermediate 30e and purified by flash C18-flash chromatography, elution gradient 0 to 90% MeCN in water (0.1% NH₄HCO₃) to give the title compound (26 mg, 47%) as a pale yellow solid. ¹H NMR δ 1.22 (3H, d), 1.64 (2H, s), 1.72-2.02 (6H, m), 2.21 (2H, d), 2.25-2.37 (1H, m), 2.44 (3H, s), 2.62 (2H, d), 2.76-2.95 (4H, m), 3.42-3.70 (9H, m), 3.89 (3H, s), 4.11-4.31 (2H, m), 4.91-5.03 (1H, m), 6.49 (1H, s), 6.78-6.93 (4H, m), 7.24 (2H, s), 8.39 (1H, s), 10.93 (1H, s), 12.67 (1H, s); m/z: ES⁺ [M+H]⁺=790.4.

### Example 93: 4-Chloro-7-[(3R*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-1H-indole-3-carbonitrile

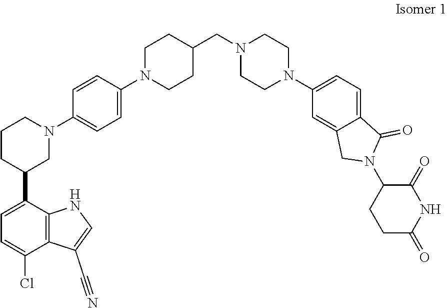 [SMILES: N#Cc1c[nH]c2c([C@H]3CCCN(c4ccc(N5CCC(CN6CCN(c7ccc8c(c7)CN(C7CCC(=O)NC7=O)C8=O)CC6)CC5)cc4)C3)ccc(Cl)c12]

The title compound was prepared using methodology described in example 24 using intermediate 91e and intermediate 1i to give the title compound (10 mg, 19%) as a pale yellow solid. ¹H NMR δ 1.22 (2H, s), 1.63 (2H, s), 1.80 (4H, d), 1.91-2.01 (2H, m), 2.22 (2H, d), 2.31-2.42 (1H, m), 2.47-2.96 (6H, m), 3.33 (7H, s), 3.44-3.61 (5H, m), 4.16-4.36 (2H, m), 4.99-5.11 (1H, m), 6.78-6.93 (4H, m), 7.06 (2H, d), 7.22 (2H, s), 7.53 (1H, d), 8.37 (1H, s), 10.95 (1H, s), 12.65 (1H, s); m/z: ES⁺ [M+H]⁺=759.3.

### Example 94

### 4-Chloro-7-[(3S*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-1H-indole-3-carbonitrile [Absolute Stereochemistry not Yet Confirmed]

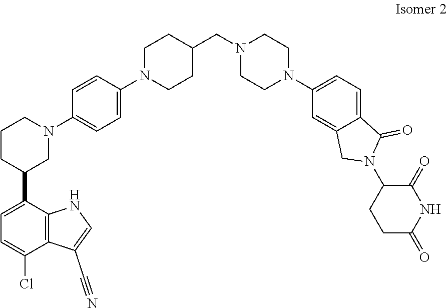 [SMILES: N#Cc1c[nH]c2c([C@H]3CCCN(c4ccc(N5CCC(CN6CCN(c7ccc8c(c7)CN(C7CCC(=O)NC7=O)C8=O)CC6)CC5)cc4)C3)ccc(Cl)c12]

The title compound was prepared using methodology described in example 24 using intermediate 92a and intermediate 1i to give the title compound (14 mg, 27%) as a pale yellow solid. ¹H NMR δ 1.22 (2H, s), 1.63 (2H, s), 1.80 (4H, d), 1.91-2.01 (2H, m), 2.22 (2H, d), 2.31-2.42 (1H, m), 2.47-2.96 (6H, m), 3.33 (7H, s), 3.44-3.61 (5H, m), 4.16-4.36 (2H, m), 4.99-5.11 (1H, m), 6.78-6.93 (4H, m), 7.06 (2H, d), 7.22 (2H, s), 7.53 (1H, d), 8.37 (1H, s), 10.95 (1H, s), 12.65 (1H, s); m/z: ES⁺ [M+H]⁺=759.3.

### Example 95

### Intermediate 95a

### 1-[4-(4,4-Dimethoxybutoxy)phenyl]piperazine

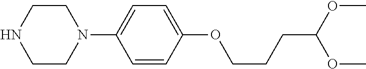 [SMILES: COC(CCCOc1ccc(N2CCNCC2)cc1)OC]

Triphenylphosphine (1.4 g, 5.61 mmol) was added to DIAD (818 μL, 4.21 mmol), 4-(piperazin-1-yl)phenol (500 mg, 2.81 mmol) and 3-(1,3-dioxolan-2-yl)propan-1-ol (556 mg, 3.37 mmol) in THF (13 mL) and was stirred at RT for 2 h under nitrogen. The solvent was evaporated to afford the crude product. The crude product was purified by flash C18-flash chromatography, elution gradient 0 to 50% MeOH in water (0.1% NH₄HCO₃) followed by ion exchange chromatography, using an SCX column. The desired product was eluted from the column using 7M NH₃/MeOH to give the title compound (0.380 g, 46.0%) as a yellow oil. ¹H NMR δ 1.63-1.73 (4H, m), 2.81 (4H, t), 2.89 (4H, d), 3.23 (6H, s), 3.89 (2H, d), 4.39 (1H, t), 6.80-6.86 (4H, m) 1H not observed; m/z: ES⁺ [M+H]⁺=295.2.

### Intermediate 95b

### 7-{4-[4-(4,4-Dimethoxybutoxy)phenyl]piperazin-1-yl}-4-methyl-1H-indole-3-carbonitrile

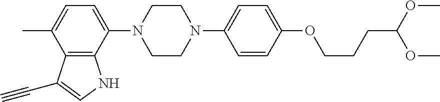 [SMILES: C#Cc1c[nH]c2c(N3CCN(c4ccc(OCCCC(OC)OC)cc4)CC3)ccc(C)c12]

The title compound was prepared using methodology described in intermediate 27c using 1-[4-(4,4-Dimethoxybutoxy)phenyl]piperazine and intermediate 8a to give the title compound (370 mg, 75%) as a white solid. ¹H NMR (CDCl₃) δ 1.83-1.90 (4H, m), 2.77 (3H, s), 3.15-3.27 (4H, m), 3.31 (5H, d), 3.37 (5H, s), 3.99 (2H, d), 4.47 (1H, t), 6.89 (2H, d), 6.99 (4H, h), 7.75 (1H, d), 8.91 (1H, s); m/z: ES⁺ [M+H]⁺=449.2.

### Example 95

### 7-{4-[4-(4-{4-[2-(2,6-Dioxopiperidin-3-yl)-1-oxo-2,3-dihydro-1H-isoindol-5-yl]piperazin-1-yl}butoxy)phenyl]piperazin-1-yl}-4-methyl-1H-indole-3-carbonitrile

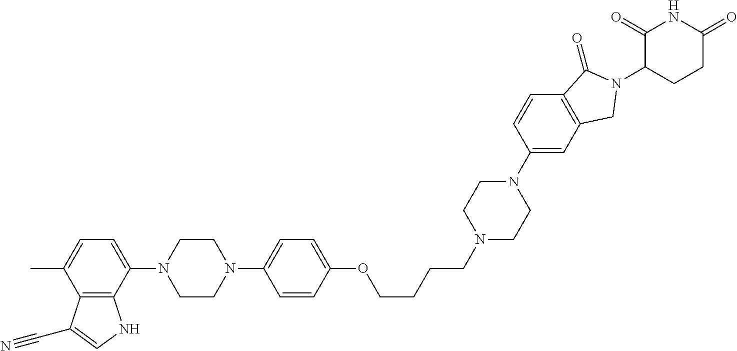 [SMILES: Cc1ccc(N2CCN(c3ccc(OCCCCN4CCN(c5ccc6c(c5)CN(C5CCC(=O)NC5=O)C6=O)CC4)cc3)CC2)c2[nH]cc(C#N)c12]

Intermediate 1i (172 mg, 0.40 mmol) was added to 7-{4-[4-(4,4-dimethoxybutoxy)phenyl]piperazin-1-yl}-4-methyl-1H-indole-3-carbonitrile (150 mg, 0.33 mmol) in formic acid (2.5 mL) under nitrogen. The resulting mixture was stirred at 40° C. for 1 h. The solvent was evaporated and diluted with DCM (1.7 mL). Sodium triacetoxyhydroborate (354 mg, 1.67 mmol) was added and the mixture was stirred at RT for 16 h. The reaction mixture was quenched with saturated NaHCO₃ solution (5 mL) and the product was extracted with DCM (5 mL), dried over MgSO₄, filtered and evaporated to afford crude product. The crude product was purified by preparative TLC (DCM:MeOH=12:1) to give the title compound (0.026 g, 10.8%) as a white solid. ¹H NMR δ 1.56-1.67 (2H, m), 1.69-1.80 (2H, m), 1.95 (1H, d), 2.22-2.42 (3H, m), 2.55 (3H, s), 2.61 (4H, s), 2.69-3.04 (2H, m), 3.11 (4H, t), 3.28 (8H, q), 3.95 (2H, t), 4.14-4.39 (2H, m), 5.05 (1H, dd), 6.75-6.98 (6H, m), 7.06 (2H, d), 7.53 (1H, d), 8.19 (1H, d), 10.94 (1H, s), 11.97 (1H, s); m/z: ES⁺ [M+H]⁺=715.4.

### Example 96

### Intermediate 96a

### 1-Benzyl-4-(4-bromophenyl)piperidine

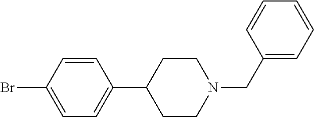 [SMILES: Brc1ccc(C2CCN(Cc3ccccc3)CC2)cc1]

Triethylamine (3.48 mL, 24.99 mmol) was added to 4-(4-bromophenyl)piperidine (2.0 g, 8.33 mmol) and (bromomethyl)benzene (2.85 g, 16.66 mmol) in THF (15 mL) under nitrogen and was stirred at RT for 16 h. The solvent was evaporated to afford crude product. The crude product was purified by flash silica chromatography, elution gradient 0 to 60% EtOAc in Et₂O to give the title compound (2.2 g, 80%) as a yellow gum. ¹H NMR δ 1.50-1.74 (4H, m), 1.93-2.07 (2H, m), 2.82-2.94 (2H, m), 3.47 (2H, s), 7.14-7.35 (7H, m), 7.39-7.52 (2H, m); m/z: ES⁺ [M+H]⁺=330.0.

### Intermediate 96b

### 7-{1-[4-(1-Benzylpiperidin-4-yl)phenyl]piperidin-3-yl}-4-chloro-1H-indole-3-carbonitrile

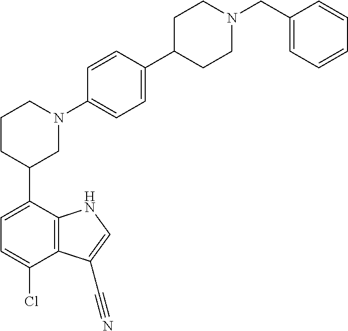 [SMILES: N#Cc1c[nH]c2c(C3CCCN(c4ccc(C5CCN(Cc6ccccc6)CC5)cc4)C3)ccc(Cl)c12]

The title compound was prepared using methodology described in intermediate 1c using 1-benzyl-4-(4-bromophenyl)piperidine and intermediate 91c to give the title compound (400 mg, 36%) as a yellow solid. ¹H NMR δ 1.6-1.68 (5H, m), 1.81 (2H, s), 1.99 (4H, s), 2.21-2.47 (2H, m), 2.68-2.81 (1H, m), 2.90 (4H, d), 3.68 (2H, d), 6.45-6.52 (1H, m), 6.85-6.9 (2H, m), 7.01-7.09 (2H, m), 7.25 (4H, d), 7.33 (2H, s), 8.39 (1H, s), 12.65 (1H, s); m/z: ES⁺ [M+H]⁺=509.3.

Intermediates 96c and 97a

1-Chloroethyl carbonochloridate (1.4 g, 9.82 mmol) was added to 7-{1-[4-(1-benzylpiperidin-4-yl)phenyl]piperidin-3-yl}-4-chloro-1H-indole-3-carbonitrile (500 mg, 0.98 mmol) in 1,2-dichloroethane (10 mL) and was stirred at 80° C. for 16 h. The solvent was evaporated to afford the crude product. The crude product was purified by flash silica chromatography, elution gradient 0 to 100% EtOAc in Et₂O to give the racemic mixture (200 mg) as a yellow solid. The enantiomers were separated by preparative HPLC on a Column: CHIRALPAK IE-3, 4.6*50 mm, 3 micron; Mobile Phase 25% Hexane:DCM (0.1% DEA): EtOH:MeCN 50%; Flow rate: 1 ml/min. to give, in order of elution, intermediate 96c (isomer 1, 70 mg, 17%, >99% ee) and intermediate 97a (isomer 2, 72 mg, 17.5%, >99% ee) as colourless gums.

### Intermediate 96c

### 4-Chloro-7-{(3R*)-1-[4-(piperidin-4-yl)phenyl]piperidin-3-yl}-1H-indole-3-carbonitrile [Absolute Stereochemistry not Yet Confirmed]

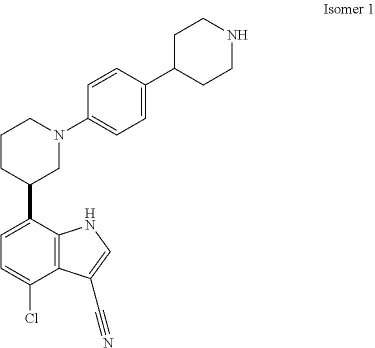

¹H NMR δ 1.43-1.54 (2H, m), 1.67 (3H, d), 1.81 (2H, s), 1.98 (2H, t), 2.58-2.68 (2H, m), 2.76 (1H, s), 2.89 (2H, t), 3.05 (2H, d), 3.69 (2H, d), 6.90 (2H, d), 7-7.07 (2H, m), 7.22 (2H, s), 8.37 (1H, s) 2H not observed; m/z: ES⁺ [M+H]⁺=419.2.

### Intermediate 97a

### 4-Chloro-7-{(3S*)-1-[4-(piperidin-4-yl)phenyl]piperidin-3-yl}-1H-indole-3-carbonitrile [Absolute Stereochemistry not Yet Confirmed]

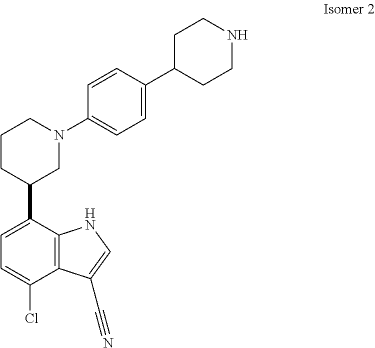

¹H NMR δ 1.4-1.49 (2H, m), 1.64 (3H, d), 1.81 (2H, s), 1.96 (1H, d), 2.44 (1H, d), 2.53-2.62 (2H, m), 2.76 (1H, t), 2.88 (1H, t), 3.01 (2H, d), 3.41 (2H, d), 3.69 (2H, d), 6.90 (2H, d), 7.04 (2H, d), 7.20 (2H, s), 8.35 (1H, s) 1H not observed; m/z: ES⁺ [M+H]⁺=419.2.

### Example 96

### 4-Chloro-7-[(3R*)-1-{4-[1-({1-[2-(2,6-dioxopiperidin-3-yl)-1-oxo-2,3-dihydro-1H-isoindol-5-yl]piperidin-4-yl}methyl)piperidin-4-yl]phenyl}piperidin-3-yl]-1H-indole-3-carbonitrile

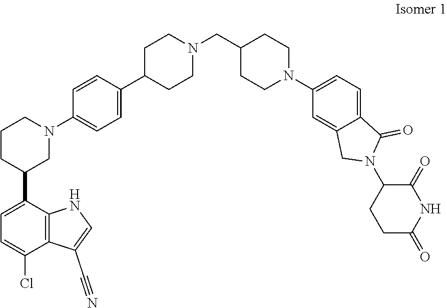 [SMILES: N#Cc1c[nH]c2c([C@H]3CCCN(c4ccc(C5CCN(CC6CCN(c7ccc8c(c7)CN(C7CCC(=O)NC7=O)C8=O)CC6)CC5)cc4)C3)ccc(Cl)c12]

Intermediate 22d (40.6 mg, 0.08 mmol) were stirred in formic acid (2 mL) at 40° C. for 1 h. The solvent was evaporated and the residue was added to NMP (2 mL) with intermediate 96c and stirred RT for 16 h. The reaction mixture was quenched with NaHCO₃ solution (10 mL) and purified by flash C18-flash chromatography, elution gradient 0 to 60% MeCN in water (0.1% NH₄HCO₃) to give the title compound (20.00 mg, 30.6%) as a white solid. ¹H NMR δ 1.12-1.25 (2H, m), 1.53-1.73 (5H, m), 1.79 (5H, d), 1.91-2.08 (4H, m), 2.21 (2H, d), 2.36 (2H, d), 2.58 (1H, d), 2.71-2.91 (5H, m), 2.96 (2H, d), 3.41 (1H, s), 3.68 (2H, d), 3.86 (2H, d), 4.19 (1H, d), 4.31 (1H, d), 5.04 (1H, dd), 6.89 (2H, d), 6.98-7.15 (4H, m), 7.22 (2H, s), 7.50 (1H, d), 8.38 (1H, s), 10.95 (1H, s), 12.67 (1H, s); m/z: ES⁺ [M+H]⁺=758.4.

### Example 97

### 4-Chloro-7-[(3S*)-1-{4-[1-({1-[2-(2,6-dioxopiperidin-3-yl)-1-oxo-2,3-dihydro-1H-isoindol-5-yl]piperidin-4-yl}methyl)piperidin-4-yl]phenyl}piperidin-3-yl]-1H-indole-3-carbonitrile [Absolute Stereochemistry not Yet Confirmed]

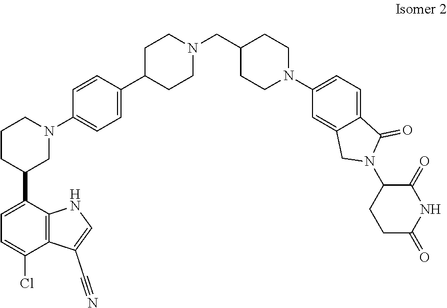 [SMILES: N#Cc1c[nH]c2c([C@H]3CCCN(c4ccc(C5CCN(CC6CCN(c7ccc8c(c7)CN(C7CCC(=O)NC7=O)C8=O)CC6)CC5)cc4)C3)ccc(Cl)c12]

The title compound was prepared using methodology described in example 96 using intermediate 97a and intermediate 22d to give the title compound (20 mg, 29%) as a white solid. ¹H NMR δ 1.11-1.25 (2H, m), 1.54-1.73 (5H, m), 1.79 (5H, d), 1.95 (4H, d), 2.18 (2H, d), 2.29-2.44 (2H, m), 2.58 (1H, d), 2.73 (2H, d), 2.81 (1H, s), 2.85 (1H, d), 2.88-2.97 (3H, m), 3.42 (1H, s), 3.68 (2H, d), 3.86 (2H, d), 4.19 (1H, d), 4.31 (1H, d), 5.04 (1H, dd), 6.89 (2H, d), 6.98-7.12 (4H, m), 7.23 (2H, s), 7.50 (1H, d), 8.38 (1H, s), 10.95 (1H, s), 12.67 (1H, s); m/z: ES⁺ [M+H]⁺=758.4.

### Example 98

### Intermediate 98a

### 3-{6-[4-(Dibutoxymethyl)piperidin-1-yl]-4-methoxy-3-oxo-1,3-dihydro-2H-pyrrolo[3,4-c]pyridin-2-yl}piperidine-2,6-dione

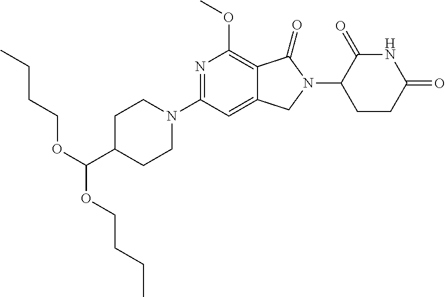 [SMILES: CCCCOC(OCCCC)C1CCN(c2cc3c(c(OC)n2)C(=O)N(C2CCC(=O)NC2=O)C3)CC1]

The title compound was prepared using methodology described in intermediate 11c using intermediate 30d and 4-(dibutoxymethyl)piperidine to give the title compound (200 mg, 30%) as a white solid. ¹H NMR δ 0.88 (6H, t), 1.15-1.24 (2H, m), 1.26-1.4 (4H, m), 1.48 (4H, dq), 1.72 (2H, d), 1.8-1.95 (2H, m), 2.23-2.39 (1H, m), 2.56 (1H, d), 2.87 (2H, dd), 3.34-3.44 (2H, m), 3.55 (3H, d), 3.87 (3H, s), 4.06-4.2 (2H, m), 4.25 (1H, d), 4.40 (2H, d), 4.95 (1H, dd), 6.47 (1H, s), 10.91 (1H, s); m/z: ES⁺ [M+H]⁺=517.5.

### Example 98

### 4-Chloro-7-[(3R*)-1-{4-[1-({1-[2-(2,6-dioxopiperidin-3-yl)-4-methoxy-3-oxo-2,3-dihydro-1H-pyrrolo[3,4-c]pyridin-6-yl]piperidin-4-yl}methyl)piperidin-4-yl]phenyl}piperidin-3-yl]-1H-indole-3-carbonitrile [Absolute Stereochemistry not Yet Confirmed]

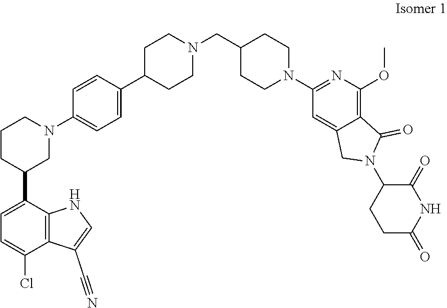 [SMILES: COc1nc(N2CCC(CN3CCC(c4ccc(N5CCC[C@H](c6ccc(Cl)c7c(C#N)c[nH]c67)C5)cc4)CC3)CC2)cc2c1C(=O)N(C1CCC(=O)NC1=O)C2]

The title compound was prepared using methodology described in example 96 using intermediate 96c and 3-{6-[4-(dibutoxymethyl)piperidin-1-yl]-4-methoxy-3-oxo-1,3-dihydro-2H-pyrrolo[3,4-c]pyridin-2-yl}piperidine-2,6-dione to give the title compound (20 mg, 30%) as a white solid. ¹H NMR δ 1.02-1.13 (2H, m), 1.51-1.72 (5H, m), 1.79 (5H, d), 1.9-2.04 (4H, m), 2.17 (2H, d), 2.26-2.41 (2H, m), 2.58 (1H, s), 2.75 (1H, s), 2.82-2.98 (6H, m), 3.43 (1H, d), 3.68 (2H, d), 3.88 (3H, s), 4.11 (1H, d), 4.25 (1H, d), 4.37 (2H, s), 4.95 (1H, dd), 6.46 (1H, s), 6.89 (2H, d), 7.06 (2H, d), 7.23 (2H, s), 8.38 (1H, s), 10.91 (1H, s), 12.67 (1H, s); m/z: ES⁺ [M+H]⁺=789.4.

### Example 99

### 4-Chloro-7-[(3S*)-1-{4-[1-({1-[2-(2,6-dioxopiperidin-3-yl)-4-methoxy-3-oxo-2,3-dihydro-1H-pyrrolo[3,4-c]pyridin-6-yl]piperidin-4-yl}methyl)piperidin-4-yl]phenyl}piperidin-3-yl]-1H-indole-3-carbonitrile [Absolute Stereochemistry not Yet Confirmed]

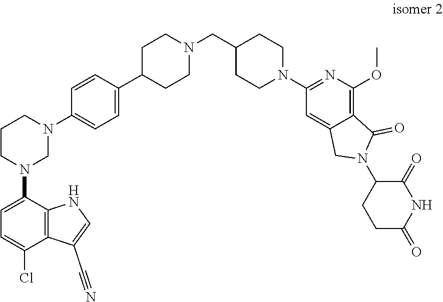

The title compound was prepared using methodology described in example 96 using intermediate 97a and intermediate 98a to give the title compound (20 mg, 29%) as a white solid. ¹H NMR δ 1.01-1.15 (2H, m), 1.52-1.72 (5H, m), 1.74-1.88 (5H, m), 1.88-2.02 (4H, m), 2.16 (2H, d), 2.32 (2H, dd), 2.58 (1H, s), 2.72 (1H, d), 2.90 (6H, q), 3.42 (1H, s), 3.68 (2H, d), 3.88 (3H, s), 4.11 (1H, d), 4.25 (1H, d), 4.37 (2H, s), 4.95 (1H, d), 6.47 (1H, s), 6.89 (2H, d), 7.06 (2H, d), 7.23 (2H, s), 8.38 (1H, s), 10.92 (1H, s), 12.68 (1H, s); m/z: ES⁺ [M+H]⁺=789.4.

### Example 100

### 4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidine-1-carbonyl]phenyl}piperidin-1-yl)-1H-indole-3-carbonitrile

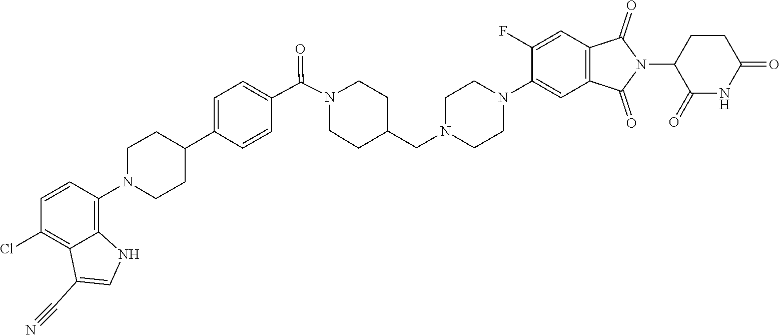 [SMILES: N#Cc1c[nH]c2c(N3CCC(c4ccc(C(=O)N5CCC(CN6CCN(c7cc8c(cc7F)C(=O)N(C7CCC(=O)NC7=O)C8=O)CC6)CC5)cc4)CC3)ccc(Cl)c12]

The title compound was prepared using methodology described in example 24 using intermediate 85c and intermediate 50b to give the title compound (20 mg, 12%) as a yellow solid. ¹H NMR δ 0.83 (1H, s), 1.09 (2H, q), 1.23 (1H, s), 1.84 (3H, s), 1.91 (2H, d), 1.97-2.07 (3H, m), 2.23 (2H, d), 2.51-2.64 (4H, m), 2.7-2.95 (5H, m), 3.03 (1H, s), 3.24 (4H, t), 3.45 (2H, d), 3.64 (1H, s), 4.46 (1H, s), 5.10 (1H, dd), 6.87 (1H, d), 7.17 (1H, d), 7.36 (4H, d), 7.45 (1H, d), 7.73 (1H, d), 8.33 (1H, s), 11.11 (1H, s), 12.29 (1H, s); m/z: ES⁺ [M+H]⁺=819.4.

### Example 101

### Intermediate 101a

### Benzyl 4-[4-(piperidin-4-yl)phenoxy]piperidine-1-carboxylate

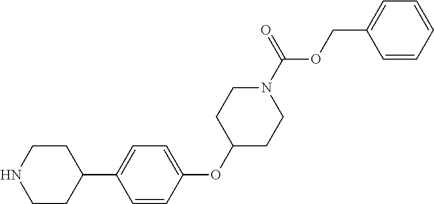 [SMILES: O=C(OCc1ccccc1)N1CCC(Oc2ccc(C3CCNCC3)cc2)CC1]

DIAD (2.10 mL, 10.82 mmol) was added to triphenylphosphine (2.84 g, 10.82 mmol), tert-butyl 4-(4-hydroxyphenyl)piperidine-1-carboxylate (2.0 g, 7.21 mmol) and 1-benzylpiperidin-4-ol (1.65 g, 8.65 mmol) in THF (20 mL) under nitrogen which was stirred at RT for 2 h. The reaction was diluted with EtOAc (50 mL), washed with NH₄Cl solution (25 mL), water (25 mL), NaCl solution (25 mL), dried over Na₂SO₄, filtered and evaporated to afford crude solid. The crude solid was stirred in formic acid (20 mL) at RT for 2 h and then evaporated to afford crude product. The crude product was purified by flash C18-flash chromatography, elution gradient 0 to 30% MeOH in water (0.1% formic acid). Product containing fractions were purified by ion exchange chromatography, using an SCX column. The product was eluted from the column using 7M NH₃/MeOH to give the title compound (300 mg, 11.8%) as a white solid. ¹H NMR δ 1.15-1.23 (2H, m), 1.4-1.47 (2H, m), 1.54-1.69 (4H, m), 1.91 (2H, t), 2.21 (2H, t), 2.61-2.7 (2H, m), 2.99 (2H, d), 3.45 (2H, d), 4.2-4.38 (1H, m), 4.77 (1H, d), 6.84 (2H, d), 7.09 (2H, d), 7.21-7.36 (5H, m) 1H not observed; m/z: ES⁺ [M+H]⁺=351.3.

### Intermediate 101b

### Benzyl 4-{4-[1-(3-cyano-4-fluoro-1H-indol-7-yl)piperidin-4-yl]phenoxy}piperidine-1-carboxylate

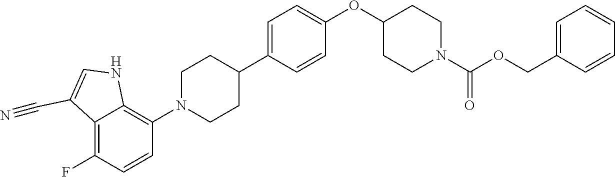 [SMILES: N#Cc1c[nH]c2c(N3CCC(c4ccc(OC5CCN(C(=O)OCc6ccccc6)CC5)cc4)CC3)ccc(F)c12]

The title compound was prepared using methodology described in intermediate 11c using benzyl 4-[4-(piperidin-4-yl)phenoxy]piperidine-1-carboxylate and intermediate 2b to give the title compound (150 mg, 41%) as a yellow solid. ¹H NMR δ 1.55-1.69 (2H, m), 1.79-1.88 (2H, m), 1.89-2 (4H, m), 2.23 (2H, t), 2.55-2.71 (3H, m), 2.71-2.81 (2H, m), 3.37 (2H, d), 3.49 (2H, s), 4.29-4.4 (1H, m), 6.82 (1H, dd), 6.88-6.96 (3H, m), 7.15-7.21 (2H, m), 7.21-7.28 (1H, m), 7.32 (4H, d), 8.27 (1H, d), 12.25 (1H, s); m/z: ES⁺ [M+H]⁺=509.3.

### Intermediate 101c

### 4-Fluoro-7-(4-{4-[(piperidin-4-yl)oxy]phenyl}piperidin-1-yl)-1H-indole-3-carbonitrile

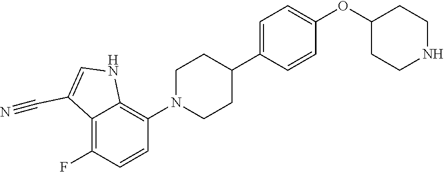 [SMILES: N#Cc1c[nH]c2c(N3CCC(c4ccc(OC5CCNCC5)cc4)CC3)ccc(F)c12]

The title compound was prepared using methodology described in intermediate 41c using benzyl 4-{4-[1-(3-cyano-4-fluoro-1H-indol-7-yl)piperidin-4-yl]phenoxy}piperidine-1-carboxylate to give the title compound (150 mg, 41%) as a yellow solid. ¹H NMR δ 0.84 (1H, d), 1.22 (4H, d), 1.85 (3H, d), 1.98 (2H, d), 2.09 (1H, s), 2.61 (1H, d), 2.76 (2H, t), 3.03 (1H, s), 3.17 (2H, d), 4.61 (1H, s), 6.82 (1H, dd), 6.88-7.04 (3H, m), 7.22 (2H, d), 8.26 (1H, s); m/z: ES⁺ [M+H]⁺=419.3.

### Example 101

### 7-[4-(4-{[1-({1-[2-(2,6-Dioxopiperidin-3-yl)-1-oxo-2,3-dihydro-1H-isoindol-5-yl]piperidin-4-yl}methyl)piperidin-4-yl]oxy}phenyl)piperidin-1-yl]-4-fluoro-1H-indole-3-carbonitrile

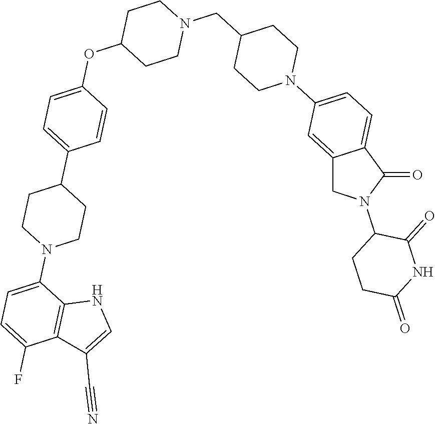 [SMILES: N#Cc1c[nH]c2c(N3CCC(c4ccc(OC5CCN(CC6CCN(c7ccc8c(c7)CN(C7CCC(=O)NC7=O)C8=O)CC6)CC5)cc4)CC3)ccc(F)c12]

Intermediate 22d (69.6 mg, 0.14 mmol) was stirred in formic acid (1.5 mL) at 40° C. for 1 h. The solvent was evaporated and the residue was added to 4-fluoro-7-(4-{4-[(piperidin-4-yl)oxy]phenyl}piperidin-1-yl)-1H-indole-3-carbonitrile (60 mg, 0.14 mmol) in NMP (1.5 mL) and stirred at RT for 16 h. The reaction mixture was purified by flash C18-flash chromatography, elution gradient 0 to 40% MeCN in water (0.1% formic acid). The fractions were combined and MeCN was removed under reduced pressure. The aqueous was basified with NaHCO₃ solution until pH 7 and extracted with DCM (6×50 mL), dried over Na₂SO₄, filtered and evaporated to afford yellow solid. The yellow solid was purified by preparative TLC (DCM:MeOH, 10:1), to give the title compound (7 mg, 6.4%) as a white solid. ¹H NMR δ 1.09-1.26 (3H, m), 1.61 (2H, d), 1.69-1.87 (5H, m), 1.87-2.03 (5H, m), 2.16 (4H, d), 2.3-2.42 (1H, m), 2.58 (2H, d), 2.64-2.98 (7H, m), 3.40 (2H, s), 3.87 (2H, d), 4.19 (1H, d), 4.32 (2H, d), 5.05 (1H, dd), 6.78 (1H, dd), 6.86-6.97 (3H, m), 7.04 (2H, d), 7.18 (2H, d), 7.50 (1H, d), 8.24 (1H, s), 10.95 (1H, s); m/z: ES⁺ [M+H]⁺=758.3.

### Intermediate 102a

### tert-Butyl 4-(5-bromo-7-methyl-1H-indol-1-yl)piperidine-1-carboxylate

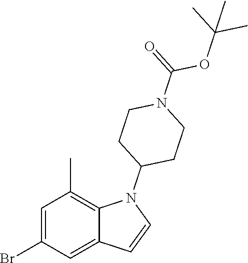 [SMILES: Cc1cc(Br)cc2ccn(C3CCN(C(=O)OC(C)(C)C)CC3)c12]

tert-Butyl 4-[(methanesulfonyl)oxy]piperidine-1-carboxylate (11.9 g, 42.84 mmol), 5-bromo-7-methyl-1H-indole (3.0 g, 14.28 mmol) and Cs₂CO₃ (13.96 g, 42.84 mmol) in DMF (57 mL) were stirred at 100° C. for 16 h. The reaction was cooled to RT and diluted with EtOAc (200 mL), washed with water (100 mL), NaCl solution (200 mL), dried with MgSO₄, filtered and evaporated to afford crude product. The crude product was purified by flash silica chromatography, elution gradient 0 to 25% EtOAc in heptane to give the title compound (1.05 g, 18.69%) as a pale yellow gum. ¹H NMR δ 1.43 (9H, s), 1.82 (2H, qd), 1.96 (2H, d), 2.69 (3H, s), 2.94 (2H, s), 4.03-4.18 (2H, m), 4.82 (1H, m), 6.43 (1H, d), 7.01 (1H, s), 7.51-7.58 (2H, m); m/z: ES⁺ [M-tBu]⁺=337.0.

### Intermediate 102b

### tert-Butyl 4-[5-(2,4-dioxo-1,3-diazinan-1-yl)-7-methyl-1H-indol-1-yl]piperidine-1-carboxylate

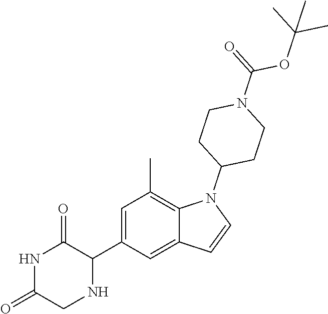 [SMILES: Cc1cc(C2NCC(=O)NC2=O)cc2ccn(C3CCN(C(=O)OC(C)(C)C)CC3)c12]

Ephos (0.143 g, 0.27 mmol) and Pd₂(dba)₃ (0.122 g, 0.13 mmol) were stirred in 1,4-dioxane (26 mL) and heated to 50° C. for 5 minutes under nitrogen. This solution was added to tert-butyl 4-(5-bromo-7-methyl-1H-indol-1-yl)piperidine-1-carboxylate (1.05 g, 2.67 mmol), 1,3-diazinane-2,4-dione (0.457 g, 4.00 mmol) and Cs₂CO₃ (2.61 g, 8.01 mmol) under nitrogen in 1,4-dioxane (20 mL) and stirred at 100° C. for 16 h. The mixture was cooled to RT, diluted with EtOAc (100 mL), washed with NaCl solution (100 mL), dried over MgSO₄, filtered and evaporated to afford crude product. The crude product was purified by flash silica chromatography, elution gradient 0 to 100% EtOAc in heptane to give the title compound (0.55 g, 48.3%) as a pale yellow solid. ¹H NMR δ 1.43 (9H, s), 1.83 (2H, qd), 1.97 (2H, s), 2.66-2.75 (5H, m), 2.96 (2H, s), 3.74 (2H, t), 4.11 (2H, d), 4.85 (1H, m), 6.45 (1H, d), 6.77-6.87 (1H, m), 7.28 (1H, d), 7.53 (1H, d), 10.23 (1H, s); m/z: ES⁺ [M-tBu]⁺=371.0.

### Example 102

### 4-Chloro-7-(4-{4-[4-({4-[5-(2,4-dioxo-1,3-diazinan-1-yl)-7-methyl-1H-indol-1-yl]piperidin-1-yl}methyl)piperidin-1-yl]phenyl}piperidin-1-yl)-1H-indole-3-carbonitrile

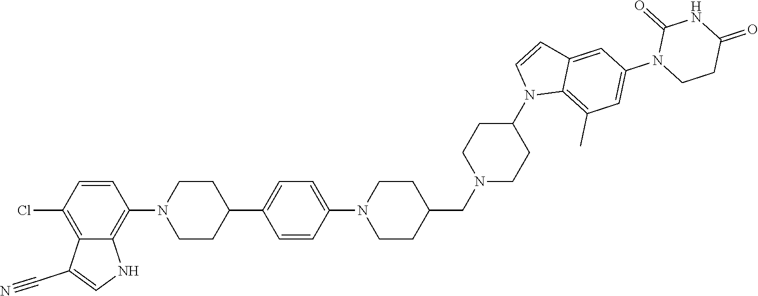 [SMILES: Cc1cc(N2CCC(=O)NC2=O)cc2ccn(C3CCN(CC4CCN(c5ccc(C6CCN(c7ccc(Cl)c8c(C#N)c[nH]c78)CC6)cc5)CC4)CC3)c12]

The title compound was prepared using methodology described in example 24 using tert-butyl 4-[5-(2,4-dioxo-1,3-diazinan-1-yl)-7-methyl-1H-indol-1-yl]piperidine-1-carboxylate and intermediate if to give the title compound (19 mg, 12%) as a white solid. ¹H NMR δ 1.23 (2H, m), 1.66 (1H, d), 1.82 (4H, dd), 1.89-2.07 (6H, m), 2.12 (2H, td), 2.24 (2H, d), 2.56-2.66 (3H, m), 2.62-2.74 (5H, m), 2.78 (2H, dd), 3.01 (2H, d), 3.38-3.45 (2H, m), 3.65 (2H, dq), 3.73 (2H, t), 4.66 (1H, td), 6.45 (1H, d), 6.79-6.95 (4H, m), 7.15 (3H, dd), 7.29 (1H, d), 7.55 (1H, d), 8.33 (1H, s), 10.26 (1H, s), 12.16 (1H, s); m/z: ES⁺ [M+H]⁺=757.4.

### Example 103

### Intermediate 103a

### tert-Butyl 4-[(3-amino-5-bromopyridin-2-yl)ethynyl]piperidine-1-carboxylate

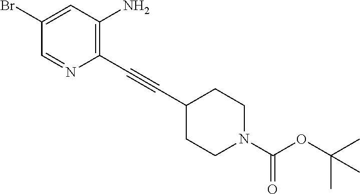 [SMILES: CC(C)(C)OC(=O)N1CCC(C#Cc2ncc(Br)cc2N)CC1]

Tetrakis(triphenylphosphine)palladium(0) (1.39 g, 1.20 mmol) was added to 5-bromo-2-iodopyridin-3-amine (3.0 g, 10.04 mmol), tert-butyl 4-ethynylpiperidine-1-carboxylate (2.1 g, 10.04 mmol) and copper(I) iodide (0.229 g, 1.20 mmol) in triethylamine (17 mL) at RT under nitrogen. The mixture was stirred at 80° C. for 16 h, cooled to RT. and the solvent was evaporated to afford crude product. The crude product was purified by flash silica chromatography, elution gradient 0 to 20% EtOAc in DCM to give the title compound (3.0 g, 79%) as a yellow solid. ¹H NMR (CDCl₃) δ 1.46 (9H, s), 1.70 (2H, dtd), 1.90 (2H, ddt), 2.86 (1H, tt), 3.19 (2H, m), 3.72-3.85 (2H, m), 4.33 (2H, s), 7.17 (1H, d), 7.98 (1H, d); m/z: ES⁺ [M+H]⁺=380.0.

### Intermediate 103b

### tert-Butyl 4-(6-bromo-1H-pyrrolo[3,2-b]pyridin-2-yl)piperidine-1-carboxylate

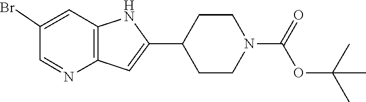 [SMILES: CC(C)(C)OC(=O)N1CCC(c2cc3ncc(Br)cc3[nH]2)CC1]

Dichlorobis(acetonitrile)palladium(II) (0.409 g, 1.58 mmol) was added to tert-butyl 4-[(3-amino-5-bromopyridin-2-yl)ethynyl]piperidine-1-carboxylate (3.0 g, 7.89 mmol) in DMF (30 mL) at RT under nitrogen. The resulting mixture was stirred at 120° C. for 16 h and then cooled to RT. The reaction mixture was diluted with EtOAc (75 mL), washed with NaHCO₃ solution (100 mL), dried over Na₂SO₄, filtered and evaporated to afford crude product. The crude product was purified by flash alumina chromatography, elution gradient 0 to 50% EtOAc in Et₂O to give the title compound (1.5 g, 50.0%) as a yellow oil. ¹H NMR δ 1.42 (9H, s), 1.96 (2H, d), 2.73 (2H, s), 2.89 (2H, s), 4.02-4.1 (3H, m), 6.35 (1H, s), 7.85 (1H, s), 8.30 (1H, s), 11.41 (1H, s); m/z: ES⁺ [M+H]⁺=380.0.

### Intermediate 103c

### tert-Butyl 4-(6-bromo-1-methyl-1H-pyrrolo[3,2-b]pyridin-2-yl)piperidine-1-carboxylate

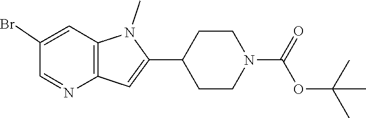 [SMILES: Cn1c(C2CCN(C(=O)OC(C)(C)C)CC2)cc2ncc(Br)cc21]

Sodium hydride (60% in mineral oil, 0.22 g, 5.52 mmol) was added to tert-butyl 4-(6-bromo-1H-pyrrolo[3,2-b]pyridin-2-yl)piperidine-1-carboxylate (1.4 g, 3.68 mmol) in DMF (15 mL) at 0° C. under nitrogen. Iodomethane (0.229 mL, 3.68 mmol) was added after 15 mins. The resulting mixture was stirred at RT for 2 h. The reaction mixture was quenched with water (1 mL) and diluted with EtOAc (50 mL). The organic layer was washed with water (150 mL), dried over Na₂SO₄, filtered and evaporated to afford crude product. The crude product was purified by flash silica chromatography, elution gradient 0 to 20% EtOAc in DCM to give the title compound (0.62 g, 42.7%) as a yellow solid. ¹H NMR (CDCl₃) δ 1.50 (9H, d), 1.64-1.78 (2H, m), 1.99 (2H, d), 2.84-2.94 (3H, m), 3.72 (3H, d), 4.30 (2H, s), 6.46 (1H, s), 7.74 (1H, s), 8.45 (1H, s); m/z: ES⁺ [M+H]⁺=394.0.

### Intermediate 103d

### tert-Butyl 4-[6-(2,4-dioxo-1,3-diazinan-1-yl)-1-methyl-1H-pyrrolo[3,2-b]pyridin-2-yl]piperidine-1-carboxylate

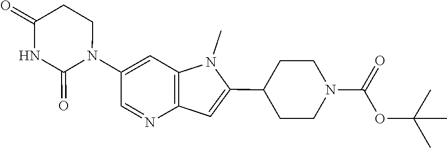 [SMILES: Cn1c(C2CCN(C(=O)OC(C)(C)C)CC2)cc2ncc(N3CCC(=O)NC3=O)cc21]

Ephos Pd G4 (280 mg, 0.30 mmol) was added to tert-butyl 4-(6-bromo-1-methyl-1H-pyrrolo[3,2-b]pyridin-2-yl)piperidine-1-carboxylate (600 mg, 1.52 mmol), Cs₂CO₃ (992 mg, 3.04 mmol), EPhos (163 mg, 0.30 mmol) and 1,3-diazinane-2,4-dione (521 mg, 4.56 mmol) in 1,4-dioxane (8 mL) under nitrogen. The mixture was stirred at 100° C. for 2 h, cooled to RT and evaporated to dryness to afford crude product. The crude product was purified by flash silica chromatography, elution gradient 0 to 4% MeOH in DCM to give the title compound (400 mg, 61.5%) as a red solid. ¹H NMR δ 1.43 (9H, s), 1.96 (2H, d), 2.77 (2H, t), 3.33 (5H, s), 3.83 (2H, t), 5.76 (1H, s), 6.40 (1H, s), 8.25 (1H, d), 10.42 (1H, s); m/z: ES⁺ [M+H]⁺=428.3.

### Example 103

### 4-Chloro-7-(4-{4-[4-({4-[6-(2,4-dioxo-1,3-diazinan-1-yl)-1-methyl-1H-pyrrolo[3,2-b]pyridin-2-yl]piperidin-1-yl}methyl)piperidin-1-yl]phenyl}piperidin-1-yl)-1H-indole-3-carbonitrile

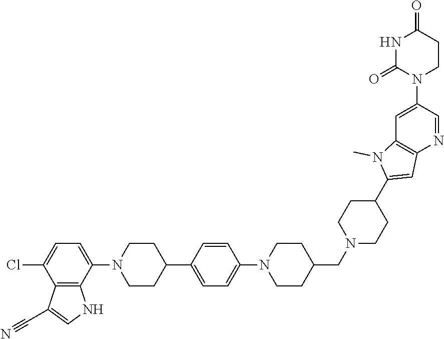 [SMILES: Cn1c(C2CCN(CC3CCN(c4ccc(C5CCN(c6ccc(Cl)c7c(C#N)c[nH]c67)CC5)cc4)CC3)CC2)cc2ncc(N3CCC(=O)NC3=O)cc21]

The title compound was prepared using methodology described in example 24 using tert-butyl 4-[6-(2,4-dioxo-1,3-diazinan-1-yl)-1-methyl-1H-pyrrolo[3,2-b]pyridin-2-yl]piperidine-1-carboxylate and intermediate if to give the title compound (20 mg, 11%) as a white solid. ¹H NMR δ 1.24 (2H, t), 1.68 (3H, q), 1.81 (4H, d), 1.92 (3H, t), 2.07 (5H, s), 2.22 (2H, d), 2.60 (3H, q), 2.67-2.78 (4H, m), 2.83 (1H, s), 2.97 (2H, d), 3.63 (2H, d), 3.73 (3H, s), 3.82 (2H, t), 6.39 (1H, s), 6.66 (1H, d), 6.89 (2H, d), 7.00 (1H, d), 7.11 (2H, d), 7.82 (1H, d), 8.12 (1H, s), 8.24 (1H, d), 10.42 (1H, s); m/z: ES⁺ [M+H]⁺=758.0.

### Example 104

### Intermediate 104a

### tert-Butyl 4-[3-fluoro-4-(methoxycarbonyl)phenyl]piperazine-1-carboxylate

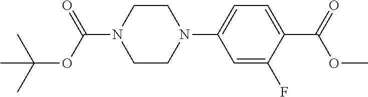 [SMILES: COC(=O)c1ccc(N2CCN(C(=O)OC(C)(C)C)CC2)cc1F]

Palladium (II) acetate (0.241 g, 1.07 mmol) was added to methyl 4-bromo-2-fluorobenzoate (5.0 g, 21.46 mmol), tert-butyl piperazine-1-carboxylate (4.4 g, 23.60 mmol), rac-2,2′-Bis(diphenylphosphino)-1,1′-binaphthyl (1.33 g, 2.15 mmol) and Cs₂CO₃ (15.3 g, 47.20 mmol) in 1,4-dioxane (20 mL) at RT under nitrogen. The mixture was stirred at 110° C. for 2 h, cooled to RT and evaporated to dryness to afford crude product. The crude product was purified by flash silica chromatography, elution gradient 0 to 30% EtOAc in Et₂O to give the title compound (6.0 g, 83%) as a pale yellow solid. ¹H NMR δ 1.42 (9H, d), 3.35 (4H, d), 3.42 (4H, s), 3.77 (3H, d), 6.67-6.84 (2H, m), 7.71 (1H, m); m/z: ES⁺ [M+H]⁺=339.0.

### Intermediate 104b

### 4-[4-(tert-Butoxycarbonyl)piperazin-1-yl]-2-fluorobenzoic acid

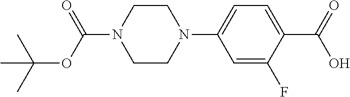 [SMILES: CC(C)(C)OC(=O)N1CCN(c2ccc(C(=O)O)c(F)c2)CC1]

LiOH (1.20 g, 50.18 mmol) was added to tert-butyl 4-[3-fluoro-4-(methoxycarbonyl)phenyl]piperazine-1-carboxylate (6.0 g, 17.73 mmol) in THF:water (1:1, 100 mL). The mixture was stirred at 60° C. for 1 h. The solvent was evaporated and the residue was acidified with HCl (2M, 25 ml). The solid was filtered and washed with water (100 mL) and dried under vacuum to give the title compound (5.0 g, 87%) as a white solid. ¹H NMR δ 1.4 (9H, s), 3.24-3.56 (8H, m), 6.7-6.88 (2H, m), 7.62-7.8 (1H, m) COOH not observed; m/z: ES⁺ [M+H]⁺=325.2.

### Intermediate 104c

### tert-Butyl 4-{4-[(2,6-dioxopiperidin-3-yl)carbamoyl]-3-fluorophenyl}piperazine-1-carboxylate

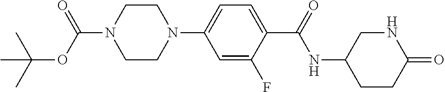 [SMILES: CC(C)(C)OC(=O)N1CCN(c2ccc(C(=O)NC3CCC(=O)NC3)c(F)c2)CC1]

Propane phosphonic acid anhydride (50% in EtOAc, 14.7 g, 23.12 mmol) was added to 4-[4-(tert-butoxycarbonyl)piperazin-1-yl]-2-fluorobenzoic acid (5.0 g, 15.42 mmol), 3-aminopiperidine-2,6-dione 2HCl (4.65 g, 23.12 mmol) and DIPEA (10.7 mL, 61.66 mmol) in DCM (100 mL) at RT. The mixture was stirred at 40° C. for 1 minute. The solvent was evaporated to afford crude product. The crude product was purified by flash silica chromatography, elution gradient 0 to 10% MeOH in DCM to give the title compound (6.0 g, 90%) as a cream solid. ¹H NMR δ 1.42 (9H, s), 1.92-2.22 (2H, m), 2.54 (1H, d), 2.78 (1H, m), 3.30 (4H, m), 3.44 (4H, m), 4.73 (1H, t), 6.82 (1H, s), 7.65 (1H, t), 8.10 (1H, dd), 9.86 (1H, s), 10.85 (1H, s); m/z: ES⁺ [M+H]⁺=435.0.

### Example 104

### 4-{4-[(1-{4-[(3S)-1-(4-chloro-3-cyano-1H-indol-7-yl)piperidin-3-yl]phenyl}piperidin-4-yl)methyl]piperazin-1-yl}-N-(2,6-dioxopiperidin-3-yl)-2-fluorobenzamide

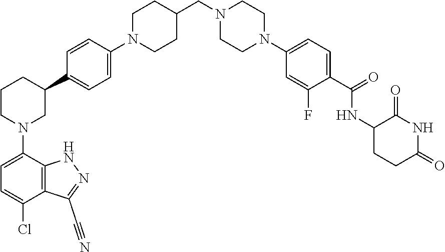 [SMILES: N#Cc1n[nH]c2c(N3CCC[C@@H](c4ccc(N5CCC(CN6CCN(c7ccc(C(=O)NC8CCC(=O)NC8=O)c(F)c7)CC6)CC5)cc4)C3)ccc(Cl)c12]

The title compound was prepared using methodology described in example 24 using tert-butyl 4-{4-[(2,6-dioxopiperidin-3-yl)carbamoyl]-3-fluorophenyl}piperazine-1-carboxylate and intermediate 11c to give the title compound (80 mg, 34%) as a white solid. ¹H NMR δ 1.21 (2H, d), 1.55 (1H, d), 1.67 (1H, s), 1.75-2.04 (6H, m), 2.06-2.16 (1H, m), 2.20 (2H, d), 2.46 (4H, s), 2.53-2.85 (6H, m), 2.97 (1H, d), 3.29 (4H, s), 3.36 (2H, s), 3.62 (2H, d), 4.73 (1H, s), 6.7-6.95 (5H, m), 7.14 (3H, dd), 7.63 (1H, t), 8.05 (1H, t), 8.29 (1H, s), 10.85 (1H, s), 12.30 (1H, s); m/z: ES⁺ [M+H]⁺=765.2.

### Example 105

### 4-{4-[(1-{4-[1-(3-Cyano-4-fluoro-1H-indol-7-yl)piperidin-4-yl]phenyl}piperidin-4-yl)methyl]piperazin-1-yl}-N-(2,6-dioxopiperidin-3-yl)-2-fluorobenzamide

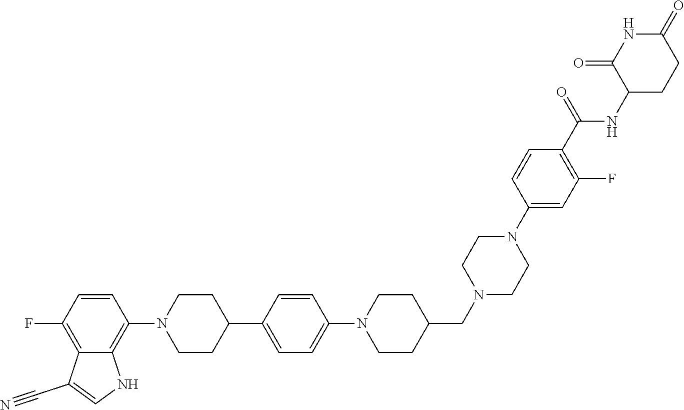 [SMILES: N#Cc1c[nH]c2c(N3CCC(c4ccc(N5CCC(CN6CCN(c7ccc(C(=O)NC8CCC(=O)NC8=O)c(F)c7)CC6)CC5)cc4)CC3)ccc(F)c12]

The title compound was prepared using methodology described in example 24 using intermediate 104c and intermediate 2c to give the title compound (82 mg, 44%) as a white solid. ¹H NMR δ 1.22 (2H, d), 1.68 (1H, s), 1.82 (4H, t), 1.89-2.05 (3H, m), 2.07-2.18 (1H, m), 2.21 (2H, d), 2.46 (4H, d), 2.60 (4H, q), 2.74 (3H, t), 3.23-3.31 (4H, m), 3.40 (2H, d), 3.63 (2H, d), 4.64-4.82 (1H, m), 6.74-6.85 (3H, m), 6.90 (3H, d), 7.12 (2H, d), 7.64 (1H, t), 8.04 (1H, t), 8.25 (1H, s), 10.85 (1H, s), 12.27 (1H, s); m/z: ES⁺ [M+H]⁺=749.2.

### Example 106

### 4-{4-[(1-{4-[1-(4-Chloro-3-cyano-1H-indol-7-yl)piperidin-4-yl]phenyl}piperidin-4-yl)methyl]piperazin-1-yl}-N-(2,6-dioxopiperidin-3-yl)-2-fluorobenzamide

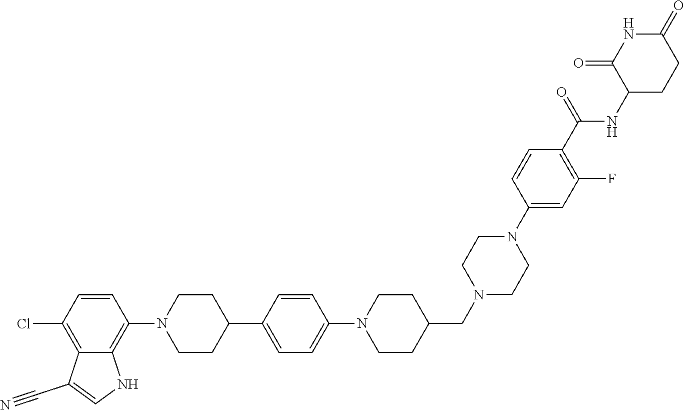 [SMILES: N#Cc1c[nH]c2c(N3CCC(c4ccc(N5CCC(CN6CCN(c7ccc(C(=O)NC8CCC(=O)NC8=O)c(F)c7)CC6)CC5)cc4)CC3)ccc(Cl)c12]

The title compound was prepared using methodology described in example 24 using intermediate 104c and intermediate if to give the title compound (55 mg, 34%) as a white solid. ¹H NMR δ 1.22 (2H, d), 1.68 (1H, s), 1.77-1.88 (4H, m), 1.89-2.05 (3H, m), 2.07-2.17 (1H, m), 2.21 (2H, d), 2.47 (4H, d), 2.54-2.7 (4H, m), 2.77 (3H, t), 3.27-3.31 (4H, m), 3.42 (2H, d), 3.64 (2H, d), 4.74 (1H, d), 6.73-6.94 (5H, m), 7.06-7.21 (3H, m), 7.63 (1H, t), 8.04 (1H, t), 8.32 (1H, s), 10.84 (1H, s), 12.27 (1H, s); m/z: ES⁺ [M+H]⁺=765.3.

### Example 107

### Intermediate 107a

### 1-Benzyl-4-[(4-bromophenyl)methyl]piperazine

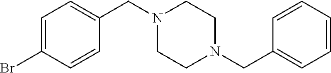 [SMILES: Brc1ccc(CN2CCN(Cc3ccccc3)CC2)cc1]

K₂CO₃ (2.84 g, 20.57 mmol) was added to 1-[(4-bromophenyl)methyl]piperazine (2.0 g, 6.86 mmol) and (bromomethyl)benzene (1.17 g, 6.86 mmol) in acetone (25 mL) at RT. The solution was stirred at RT for 2 h and was then diluted with EtOAc (100 mL), washed with NH₄Cl solution (50 mL), NaCl solution (50 mL), dried over Na₂SO₄, filtered and evaporated to afford crude product. The crude product was purified by flash silica chromatography, elution gradient 0 to 30% EtOAc in Et₂O to give the title compound (1.5 g, 63.3%) as a white solid. ¹H NMR δ 2.36 (8H, s), 3.43 (4H, d), 7.17-7.36 (7H, m), 7.44-7.55 (2H, m); m/z: ES⁺ [M+H]=344.9.

### Intermediate 107b

### tert-Butyl 4-{4-[(4-benzylpiperazin-1-yl)methyl]phenyl}-3,6-dihydropyridine-1(2H)-carboxylate

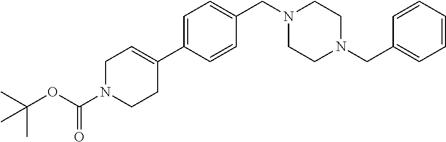 [SMILES: CC(C)(C)OC(=O)N1CC=C(c2ccc(CN3CCN(Cc4ccccc4)CC3)cc2)CC1]

PdCl₂(dtbpf) (0.264 g, 0.41 mmol) was added to K₂CO₃ (1.68 g, 12.16 mmol), 1-benzyl-4-[(4-bromophenyl)methyl]piperazine (1.4 g, 4.05 mmol) and tert-butyl 4-(4,4,5,5-tetramethyl-1,3,2-dioxaborolan-2-yl)-3,6-dihydropyridine-1(2H)-carboxylate (1.37 g, 4.46 mmol) in 1,4-dioxane:water (4:1, 20 mL) at RT under nitrogen. The mixture was stirred at 80° C. for 1 h, cooled to RT, filtered through Celite® and the solvent was evaporated to dryness to afford crude product. The crude product was purified by flash silica chromatography, elution gradient 0 to 60% EtOAc in Et₂O to give the title compound (1.5 g, 83%) as a brown solid; m/z: ES⁺ [M+H]⁺=448.1. no nmr data Intermediate 107c

### 1-Benzyl-4-{[4-(1,2,3,6-tetrahydropyridin-4-yl)phenyl]methyl}piperazine

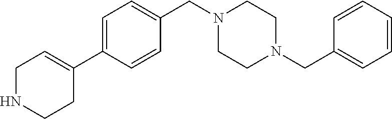 [SMILES: C1=C(c2ccc(CN3CCN(Cc4ccccc4)CC3)cc2)CCNC1]

tert-Butyl 4-{4-[(4-benzylpiperazin-1-yl)methyl]phenyl}-3,6-dihydropyridine-1(2H)-carboxylate (1.35 g, 3.02 mmol) was added to DCM (10 mL) and TFA (10 mL) at RT under nitrogen. The mixture was stirred at RT for 1 h and then evaporated to dryness to afford crude product. The crude product was purified by ion exchange chromatography, using an SCX column. The desired product was eluted from the column using 7M NH₃/MeOH to give the title compound (0.95 g, 91%) as a pale yellow solid. ¹H NMR δ 1.24 (1H, s), 2.00 (1H, s), 2.36 (9H, s), 2.96 (2H, t), 3.42 (6H, d), 6.17 (1H, s), 7.1-7.48 (9H, m); m/z: ES⁺ [M+H]⁺=348.1.

### Intermediate 107d

### 7-[4-{4-[(4-Benzylpiperazin-1-yl)methyl]phenyl}-3,6-dihydropyridin-1(2H)-yl]-4-fluoro-1H-indole-3-carbonitrile

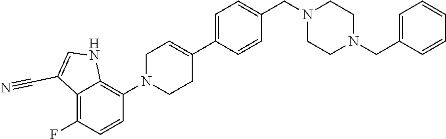 [SMILES: N#Cc1c[nH]c2c(N3CC=C(c4ccc(CN5CCN(Cc6ccccc6)CC5)cc4)CC3)ccc(F)c12]

The title compound was prepared using methodology described in intermediate 63d using 1-benzyl-4-{[4-(1,2,3,6-tetrahydropyridin-4-yl)phenyl]methyl}piperazine and intermediate 2b to give the title compound (850 mg, 65%) as a yellow solid. ¹H NMR δ 0.73-0.91 (2H, m), 2.38 (8H, s), 2.70 (2H, s), 3.29 (2H, t), 3.74 (2H, q), 6.33 (1H, d), 6.83-7.02 (2H, m), 7.19-7.37 (9H, m), 7.45 (2H, d), 8.26 (1H, s), 12.35 (1H, s); m/z: ES⁺ [M+H]⁺=506.0.

### Intermediate 107e

### 4-Fluoro-7-(4-{4-[(piperazin-1-yl)methyl]phenyl}piperidin-1-yl)-1H-indole-3-carbonitrile

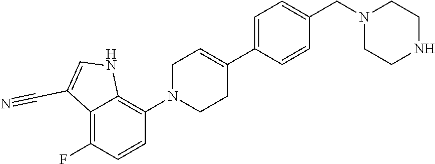 [SMILES: N#Cc1c[nH]c2c(N3CC=C(c4ccc(CN5CCNCC5)cc4)CC3)ccc(F)c12]

The title compound was prepared using methodology described in intermediate 41c using 7-[4-{4-[(4-benzylpiperazin-1-yl)methyl]phenyl}-3,6-dihydropyridin-1(2H)-yl]-4-fluoro-1H-indole-3-carbonitrile to give the title compound (250 mg, 36%) as a yellow solid. ¹H NMR δ 0.81 (2H, q), 1.79-2.08 (4H, m), 2.29 (2H, s), 2.44 (2H, s), 2.77 (2H, t), 2.86 (4H, s), 3.3-3.53 (4H, m), 6.82 (1H, dd), 6.93 (1H, dd), 7.17 (2H, q), 7.22-7.37 (3H, m), 8.27 (1H, s); m/z: ES⁺ [M+H]⁺=418.1.

### Example 107

### 7-[4-(4-{[4-({1-[2-(2,6-Dioxopiperidin-3-yl)-1-oxo-2,3-dihydro-1H-isoindol-5-yl]piperidin-4-yl}methyl)piperazin-1-yl]methyl}phenyl)piperidin-1-yl]-4-fluoro-1H-indole-3-carbonitrile

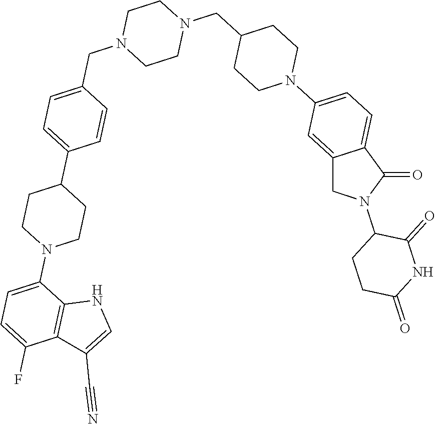 [SMILES: N#Cc1c[nH]c2c(N3CCC(c4ccc(CN5CCN(CC6CCN(c7ccc8c(c7)CN(C7CCC(=O)NC7=O)C8=O)CC6)CC5)cc4)CC3)ccc(F)c12]

The title compound was prepared using methodology described in example 24 using 4-fluoro-7-(4-{4-[(piperazin-1-yl)methyl]phenyl}piperidin-1-yl)-1H-indole-3-carbonitrile and intermediate 22d to give the title compound (20 mg, 9%) as a white solid. ¹H NMR δ 1.1-1.23 (2H, m), 1.66-1.81 (3H, m), 1.81-2 (5H, m), 2.13 (2H, d), 2.36 (8H, s), 2.53-2.74 (5H, m), 2.75-2.95 (3H, m), 3.42 (2H, s), 3.85 (4H, d), 4.19 (1H, d), 4.31 (1H, d), 5.04 (1H, dd), 6.46 (1H, s), 6.63 (1H, s), 7.03 (2H, d), 7.24 (4H, s), 7.49 (1H, d), 7.92 (1H, s), 10.93 (1H, s) 1H not observed; m/z: ES⁺ [M+H]⁺=757.5.

### Example 108

### Intermediate 108a

### 5-Bromo-2-[4-(dibutoxymethyl)piperidin-1-yl]pyridine

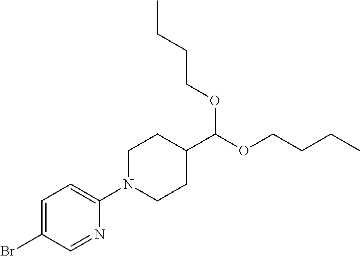 [SMILES: CCCCOC(OCCCC)C1CCN(c2ccc(Br)cn2)CC1]

The title compound was prepared using methodology described in intermediate 63a using 5-bromo-2-fluoropyridine and 4-(dibutoxymethyl)piperidine to give the title compound (1.9 g, 84%) as a colourless gum. ¹H NMR δ 0.93 (6H, t), 1.37-1.43 (4H, m), 1.53-1.6 (4H, m), 1.84 (4H, t), 2.72-2.79 (3H, m), 3.39-3.47 (2H, m), 3.63 (2H, dt), 4.17 (1H, d), 4.26 (2H, d), 6.63 (1H, d), 7.35 (1H, dd), 8.05 (1H, d); m/z: ES⁺ [M+H]⁺=399.1.

### Intermediate 108b

### Benzyl 6-[4-(dibutoxymethyl)piperidin-1-yl]-3′,6′-dihydro[3,4′-bipyridine]-1′(2′H)-carboxylate

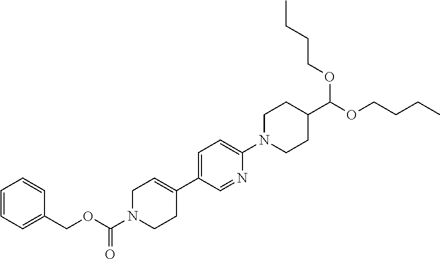 [SMILES: CCCCOC(OCCCC)C1CCN(c2ccc(C3=CCN(C(=O)OCc4ccccc4)CC3)cn2)CC1]

The title compound was prepared using methodology described in intermediate 63b using 5-bromo-2-[4-(dibutoxymethyl)piperidin-1-yl]pyridine and benzyl 4-(4,4,5,5-tetramethyl-1,3,2-dioxaborolan-2-yl)-3,6-dihydropyridine-1(2H)-carboxylate to give the title compound (1.7 g, 75%) as a brown solid. ¹H NMR δ 0.94 (6H, t), 1.31-1.46 (7H, m), 1.53-1.62 (4H, m), 1.82-1.94 (3H, m), 2.50 (2H, s), 2.83 (2H, t), 3.45 (2H, dt), 3.64 (2H, dt), 3.72 (2H, dd), 4.15-4.19 (2H, m), 4.34 (2H, d), 5.19 (2H, s), 5.92 (1H, d), 6.65 (1H, d), 7.3-7.42 (5H, m), 7.51 (1H, dd), 8.11-8.29 (1H, m); m/z: ES⁺ [M+H]⁺=536.1.

### Intermediate 108c

### 2-[4-(Dibutoxymethyl)piperidin-1-yl]-5-(piperidin-4-yl)pyridine

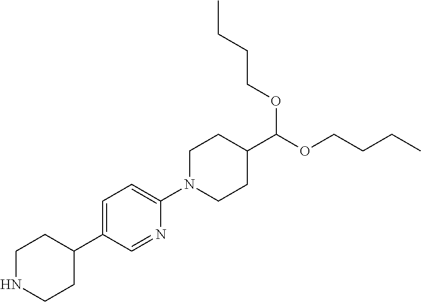 [SMILES: CCCCOC(OCCCC)C1CCN(c2ccc(C3CCNCC3)cn2)CC1]

The title compound was prepared using methodology described in intermediate 63c using benzyl 6-[4-(dibutoxymethyl)piperidin-1-yl]-3′,6′-dihydro[3,4′-bipyridine]-1′(2′H)-carboxylate to give the title compound (1.0 g, 83%) as a yellow gum. ¹H NMR δ (CDCl₃) δ 0.93 (6H, t), 1.23-1.48 (7H, m), 1.5-1.71 (6H, m), 1.74-1.9 (5H, m), 2.45-2.57 (1H, m), 2.69-2.81 (4H, m), 3.23 (2H, s), 3.44 (2H, dt), 3.63 (2H, dt), 4.17 (1H, d), 4.26 (2H, d), 6.63 (1H, d), 7.35 (1H, dd), 8.05 (1H, d); m/z: ES⁺ [M+H]⁺=404.2.

### Intermediate 108d

### 4-Chloro-7-(4-{6-[4-(dibutoxymethyl)piperidin-1-yl]pyridin-3-yl}piperidin-1-yl)-1H-indole-3-carbonitrile

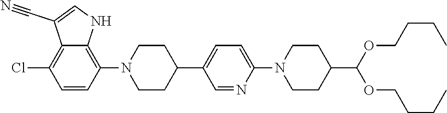 [SMILES: CCCCOC(OCCCC)C1CCN(c2ccc(C3CCN(c4ccc(Cl)c5c(C#N)c[nH]c45)CC3)cn2)CC1]

The title compound was prepared using methodology described in intermediate 63d using 2-[4-(dibutoxymethyl)piperidin-1-yl]-5-(piperidin-4-yl)pyridine and intermediate 1b to give the title compound (0.6 g, 51%) as a yellow solid. ¹H NMR δ (CDCl₃) δ 0.94 (6H, t), 1.32-1.46 (6H, m), 1.53-1.63 (4H, m), 1.8-2.01 (7H, m), 2.55-2.69 (1H, m), 2.74-2.83 (2H, m), 2.83-2.95 (2H, m), 3.34-3.53 (4H, m), 3.55-3.7 (2H, m), 4.18 (1H, d), 4.28 (2H, d), 6.67 (1H, d), 6.90 (1H, d), 7.17 (1H, d), 7.34-7.44 (1H, m), 7.78 (1H, d), 8.11 (1H, d), 9.59 (1H, s); m/z: ES⁺ [M+H]⁺=578.3.

### Example 108

### 4-Chloro-7-(4-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl)-1H-indole-3-carbonitrile

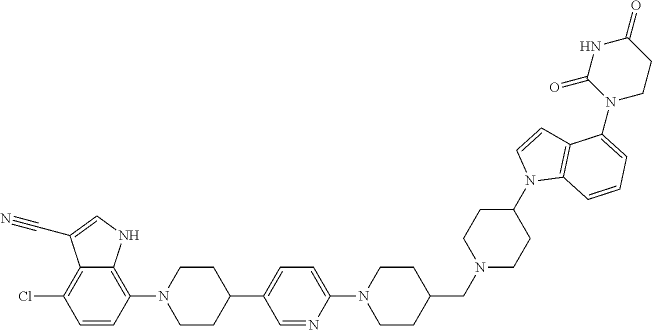 [SMILES: N#Cc1c[nH]c2c(N3CCC(c4ccc(N5CCC(CN6CCC(n7ccc8c(N9CCC(=O)NC9=O)cccc87)CC6)CC5)nc4)CC3)ccc(Cl)c12]

The title compound was prepared using methodology described in example 23 using 4-chloro-7-(4-{6-[4-(dibutoxymethyl)piperidin-1-yl]pyridin-3-yl}piperidin-1-yl)-1H-indole-3-carbonitrile and intermediate 5f to give the title compound (92 mg, 48%) as a white solid. ¹H NMR δ 1.11 (2H, d), 1.68-1.88 (5H, m), 1.88-2.08 (6H, m), 2.1-2.33 (4H, m), 2.53-2.63 (1H, m), 2.66-2.87 (6H, m), 3.00 (2H, d), 3.43 (2H, d), 3.78 (2H, t), 4.13-4.48 (3H, m), 6.42 (1H, d), 6.82 (2H, dd), 6.96 (1H, d), 7.14 (2H, t), 7.38-7.61 (3H, m), 8.04 (1H, d), 8.31 (1H, s), 10.33 (1H, s), 12.31 (1H, s); m/z: ES⁺ [M+H]⁺=744.8.

### Example 109

### 4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridin-3-yl}piperidin-1-yl)-1H-indole-3-carbonitrile

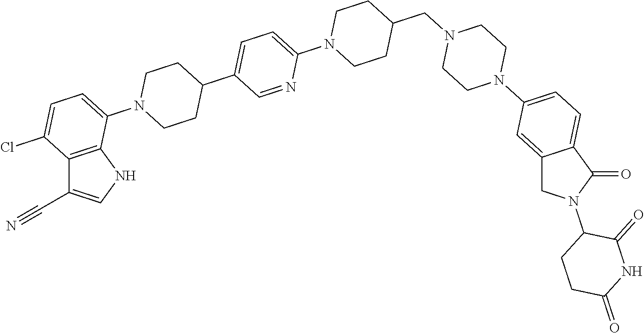 [SMILES: N#Cc1c[nH]c2c(N3CCC(c4ccc(N5CCC(CN6CCN(c7ccc8c(c7)CN(C7CCC(=O)NC7=O)C8=O)CC6)CC5)nc4)CC3)ccc(Cl)c12]

The title compound was prepared using methodology described in example 24 using intermediate 108d and intermediate 1i to give the title compound (74 mg, 28%) as a white solid. ¹H NMR δ 1.10 (2H, d), 1.80 (5H, t), 1.85-2.02 (3H, m), 2.20 (2H, d), 2.36 (2H, dd), 2.58 (4H, d), 2.64-2.82 (4H, m), 2.90 (1H, t), 3.29 (5H, s), 3.51 (2H, d), 4.06-4.4 (4H, m), 5.05 (1H, dd), 6.69-6.9 (2H, m), 7.08 (3H, q), 7.43 (1H, d), 7.52 (1H, d), 8.04 (1H, s), 8.25 (1H, s), 10.95 (1H, s), 12.31 (1H, s); m/z: ES⁺ [M+H]⁺=760.4.

### Example 110

### 4-Chloro-7-{4-[4-(4-{4-[2-(2,6-dioxopiperidin-3-yl)-1-oxo-2,3-dihydro-1H-isoindol-5-yl]piperazin-1-yl}piperidin-1-yl)phenyl]piperidin-1-yl}-1H-indole-3-carbonitrile

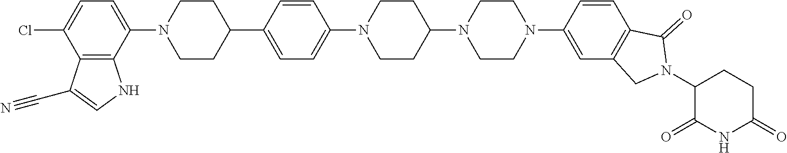 [SMILES: N#Cc1c[nH]c2c(N3CCC(c4ccc(N5CCC(N6CCN(c7ccc8c(c7)CN(C7CCC(=O)NC7=O)C8=O)CC6)CC5)cc4)CC3)ccc(Cl)c12]

Intermediate 86c (130 mg, 0.30 mmol) and intermediate 1i (129 mg, 0.30 mmol) was stirred in formic acid (1.0 mL) at 40° C. for 1 h. The mixture was evaporated to dryness and the residue was diluted with DMF (4 mL). Titanium isopropoxide (85 mg, 0.30 mmol) and sodium triacetoxyborohydride (764 mg, 3.60 mmol) was added and stirred at RT for 16 h. Purification of the reaction mixture by preparative HPLC (Column A, Eluent A) gave the title compound in the form of a formate salt (8.9 mg, 4%) as a pale yellow solid. ¹H NMR δ 0.73-0.9 (2H, m), 1.22 (3H, s), 1.53 (2H, d), 1.82 (3H, s), 1.87 (4H, s), 2.57 (1H, d), 2.65 (5H, s), 2.72 (2H, t), 2.83 (OH, d), 3.26 (4H, s), 3.53-3.8 (4H, m), 4.13-4.39 (2H, m), 4.98 (1H, dd), 6.58 (1H, d), 6.89 (3H, dd), 7.01 (2H, d), 7.10 (2H, d), 7.48 (1H, d), 7.98 (1H, s), 10.76 (1H, s); m/z: ES⁺ [M+H]⁺=745.3.

### Example 111 4-Chloro-7-[4-(4-{4-[4-(2,4-dioxo-1,3-diazinan-1-yl)-1H-indol-1-yl][1,4′-bipiperidin]-1′-yl}phenyl)piperidin-1-yl]-1H-indole-3-carbonitrile

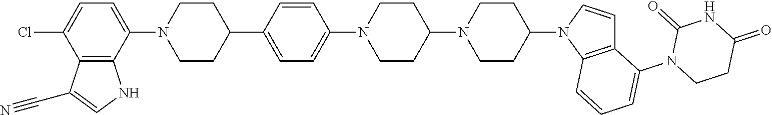 [SMILES: N#Cc1c[nH]c2c(N3CCC(c4ccc(N5CCC(N6CCC(n7ccc8c(N9CCC(=O)NC9=O)cccc87)CC6)CC5)cc4)CC3)ccc(Cl)c12]

The title compound was prepared using methodology described in example 110 using intermediate 86c and intermediate 5f to give the title compound in the form of a formate salt (17 mg, 5%) as a white solid. ¹H NMR δ 0.84 (1H, s), 1.24 (1H, s), 1.85 (3H, d), 1.91-2.04 (2H, m), 2.07 (1H, s), 2.19 (3H, s), 2.51 (5H, s), 2.67-2.84 (5H, m), 3.43 (3H, d), 3.67 (2H, s), 3.74-3.89 (4H, m), 4.79 (1H, s), 6.45-6.54 (1H, m), 6.87 (1H, d), 6.99 (3H, dd), 7.14-7.24 (4H, m), 7.42 (1H, s), 7.58 (1H, d), 8.33 (1H, d), 10.34 (1H, s), 12.27 (1H, s); m/z: ES⁺ [M+H]⁺=729.3.

### Example 112

### Intermediate 112a

### 4-Chloro-7-(4-{4-[4-(dibutoxymethyl)piperidin-1-yl]phenyl}piperidin-1-yl)-1-methyl-1H-indole-3-carbonitrile

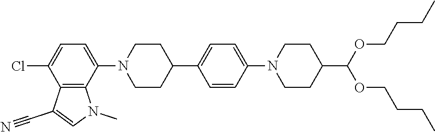 [SMILES: CCCCOC(OCCCC)C1CCN(c2ccc(C3CCN(c4ccc(Cl)c5c(C#N)cn(C)c45)CC3)cc2)CC1]

The title compound was prepared using methodology described in intermediate 57b using intermediate 143b and 7-Bromo-4-chloro-1-methyl-1H-indole-3-carbonitrile to give the title compound (90 mg, 41%) as a white solid. ¹H NMR (CDCl₃) δ 0.96 (7H, t), 1.28 (1H, d), 1.34-1.52 (4H, m), 1.5-1.85 (5H, m), 1.96 (6H, dt), 2.58-2.75 (3H, m), 2.91 (2H, td), 3.27 (2H, d), 3.47 (2H, dt), 3.67 (4H, dq), 4.25 (4H, d), 6.99 (3H, dd), 7.18 (3H, t), 7.54 (1H, s); m/z: ES⁺ [M+H]⁺=591.5.

### Example 112

### 4-chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1-methyl-1H-indole-3-carbonitrile

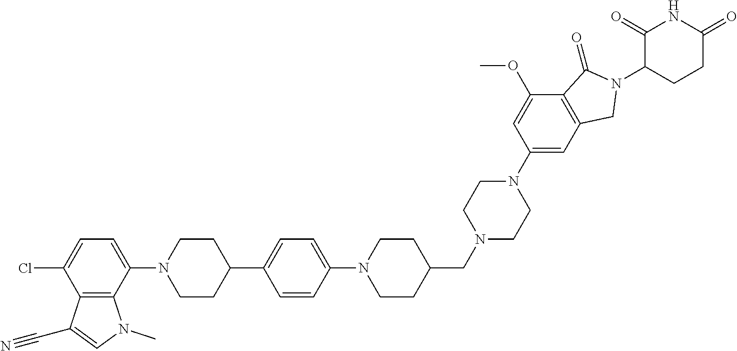 [SMILES: COc1cc(N2CCN(CC3CCN(c4ccc(C5CCN(c6ccc(Cl)c7c(C#N)cn(C)c67)CC5)cc4)CC3)CC2)cc2c1C(=O)N(C1CCC(=O)NC1=O)C2]

The title compound was prepared using methodology described in example 24 using 4-chloro-7-(4-{4-[4-(dibutoxymethyl)piperidin-1-yl]phenyl}piperidin-1-yl)-1-methyl-1H-indole-3-carbonitrile and intermediate 7c to give the title compound (55 mg, 35%) as a white solid. ¹H NMR δ 1.23 (3H, d), 1.69 (1H, s), 1.77-1.99 (8H, m), 2.19-2.38 (3H, m), 2.62 (4H, t), 2.79-2.93 (3H, m), 3.21 (2H, d), 3.31 (5H, s), 3.63 (2H, d), 3.84 (3H, s), 4.11 (1H, d), 4.21 (4H, s), 4.27 (1H, s), 4.97 (1H, dd), 6.49 (1H, s), 6.61 (1H, s), 6.89 (2H, d), 7.07-7.19 (3H, m), 7.22 (1H, d), 8.30 (1H, s), 10.92 (1H, s); m/z: ES⁺ [M+H]⁺=803.3.

### Example 113

### Intermediate 113a

### Benzyl 4-(3-bromophenyl)piperidine-1-carboxylate

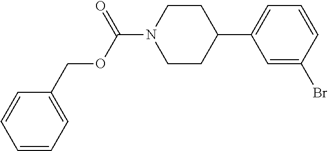 [SMILES: O=C(OCc1ccccc1)N1CCC(c2cccc(Br)c2)CC1]

Benzyl carbonochloridate (1.13 mL, 7.95 mmol) was added to 4-(3-bromophenyl)piperidine, HCl (2.0 g, 7.23 mmol) and Na₂CO₃ (2.29 g, 21.69 mmol) in water:THF (1:1, 40 mL) and was stirred at RT for 16 h. The solvent was evaporated to afford crude product. The crude product was purified by flash silica chromatography, elution gradient 0 to 40% EtOAc in Et₂O to give the title compound (1.5 g, 55.4%) as a colourless oil. ¹H NMR (CDCl₃) δ 1.65 (3H, tt), 1.86 (2H, d), 2.67 (1H, tt), 2.90 (2H, t), 4.36 (2H, d), 5.18 (2H, s), 7.1-7.24 (2H, m), 7.37 (2H, dt), 7.38-7.43 (4H, m); m/z: ES⁺ [M+H]⁺=373.7.

### Intermediate 113b

### Benzyl 4-{3-[4-(dibutoxymethyl)piperidin-1-yl]phenyl}piperidine-1-carboxylate

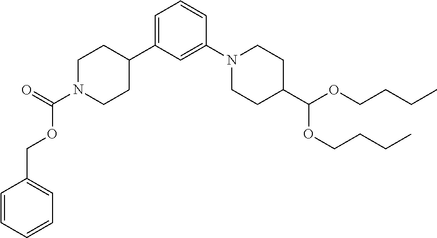 [SMILES: CCCCOC(OCCCC)C1CCN(c2cccc(C3CCN(C(=O)OCc4ccccc4)CC3)c2)CC1]

The title compound was prepared using methodology described in intermediate 11a using benzyl 4-(3-bromophenyl)piperidine-1-carboxylate and 4-(dibutoxymethyl)piperidine to give the title compound (1.1 g, 55%) as a yellow oil. ¹H NMR (CDCl₃) δ 0.95 (6H, t), 1.23-1.33 (1H, m), 1.33-1.53 (5H, m), 1.55-1.72 (6H, m), 1.87 (5H, t), 2.54-2.76 (3H, m), 2.89 (2H, s), 3.47 (2H, dt), 3.55-3.82 (4H, m), 4.22 (1H, d), 4.35 (2H, s), 5.18 (2H, s), 6.63-6.85 (2H, m), 7.16-7.25 (1H, m), 7.31-7.43 (6H, m); m/z: ES⁺ [M+H]⁺=537.2.

### Intermediate 113c

### 4-(Dibutoxymethyl)-1-[3-(piperidin-4-yl)phenyl]piperidine

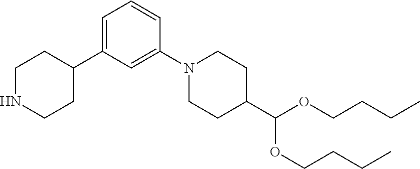 [SMILES: CCCCOC(OCCCC)C1CCN(c2cccc(C3CCNCC3)c2)CC1]

The title compound was prepared using methodology described in intermediate 11b using benzyl 4-{3-[4-(dibutoxymethyl)piperidin-1-yl]phenyl}piperidine-1-carboxylate to give the title compound (0.5 g, 66%) as a colourless gum. ¹H NMR (CDCl₃) δ 0.95 (6H, t), 1.35-1.54 (6H, m), 1.54-1.63 (4H, m), 1.69-1.85 (1H, m), 1.90 (2H, d), 2.07 (4H, d), 2.64-2.76 (3H, m), 3.02 (2H, s), 3.41-3.61 (4H, m), 3.61-3.75 (4H, m), 4.21 (1H, d), 6.72 (1H, d), 6.79-6.88 (2H, m), 7.22 (1H, t); m/z: ES⁺ [M+Na]⁺=403.1.

### Intermediate 113d

### 4-Chloro-7-(4-{3-[4-(dibutoxymethyl)piperidin-1-yl]phenyl}piperidin-1-yl)-1H-indole-3-carbonitrile

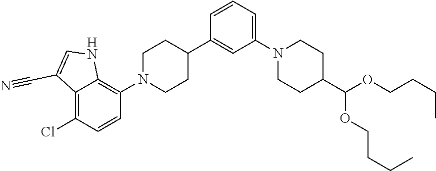 [SMILES: CCCCOC(OCCCC)C1CCN(c2cccc(C3CCN(c4ccc(Cl)c5c(C#N)c[nH]c45)CC3)c2)CC1]

LHMDS (1M in THF, 3.48 mL, 3.48 mmol) was added to 4-(dibutoxymethyl)-1-[3-(piperidin-4-yl)phenyl]piperidine (400 mg, 0.99 mmol), intermediate 1b (254 mg, 0.99 mmol) and Pd-PEPPSI-IHept^{Cl} (97 mg, 0.10 mmol) in 1,4-dioxane (4 mL) under nitrogen. The mixture was stirred at 80° C. for 2 h, cooled to RT And quenched with NH₄Cl solution (10 mL), extracted with EtOAc (30 mL), dried over Na₂SO₄, filtered and evaporated to afford crude product. The crude product was purified by flash C18-flash chromatography, elution gradient 0 to 100% MeOH in water (0.1% formic acid) to give the title compound (120 mg, 20.93%) as a yellow gum. ¹H NMR (CDCl₃) δ 0.96 (6H, t), 1.25-1.32 (3H, m), 1.41 (5H, dt), 1.55-1.68 (6H, m), 1.82 (2H, s), 1.94 (2H, s), 2.77 (3H, d), 2.96 (2H, s), 3.39-3.53 (4H, m), 3.6-3.81 (4H, m), 4.25 (1H, d), 6.95 (3H, d), 7.20 (1H, d), 7.32 (1H, s), 7.80 (1H, s) 1H not observed; m/z: ES⁺ [M+H]⁺=577.1.

### Example 113

### 4-Chloro-7-(4-{3-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile

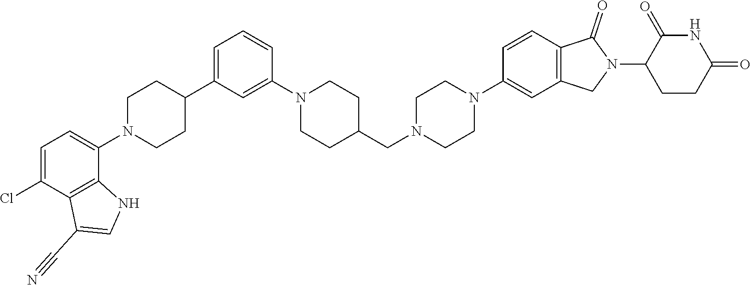 [SMILES: N#Cc1c[nH]c2c(N3CCC(c4cccc(N5CCC(CN6CCN(c7ccc8c(c7)CN(C7CCC(=O)NC7=O)C8=O)CC6)CC5)c4)CC3)ccc(Cl)c12]

The title compound was prepared using methodology described in example 24 using 4-chloro-7-(4-{3-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile and intermediate 1i to give the title compound (42 mg, 32%) as a white solid. ¹H NMR δ 1.23 (3H, d), 1.71 (1H, s), 1.83 (4H, t), 1.97 (3H, t), 2.23 (2H, d), 2.37 (1H, d), 2.54-2.8 (8H, m), 2.85-2.97 (1H, m), 3.29 (4H, s), 3.61 (2H, s), 3.69 (3H, d), 4.21 (1H, d), 4.33 (1H, d), 5.05 (1H, dd), 6.66-6.83 (3H, m), 6.85 (1H, s), 6.97-7.13 (3H, m), 7.16 (1H, d), 7.52 (1H, d), 8.19 (1H, s), 10.95 (1H, s), 12.28 (1H, s); m/z: ES⁺ [M+H]⁺=759.9.

### Example 114

### Intermediate 114a

### 3-Bromo-2-(prop-1-yn-1-yl)aniline

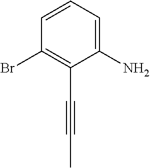

PdCl₂(PPh₃)₂ (4.72 g, 6.73 mmol) was added to a degassed mixture of 3-bromo-2-iodoaniline (20.05 g, 67.30 mmol), but-2-ynoic acid (16.97 g, 201.90 mmol) and 2,3,4,6,7,8,9,10-octahydropyrimido[1,2-a]azepine (27.7 mL, 201.90 mmol) in DMSO (200 mL). The reaction was stirred at 80° C. for 3 h. The reaction was cooled to RT and diluted with water (500 mL). The product was extracted with DCM (1 L), washed with NaCl solution (500 mL), dried over Na₂SO₄, filtered and evaporated to give crude product. The crude product was purified by flash silica chromatography, elution gradient 0 to 30% EtOAc in heptane to give the title compound (6.72 g, 47.5%) as a pale brown gum. ¹H NMR (CDCl₃) δ 4.30 (2H, s), 6.61 (1H, dd), 6.86-6.94 (2H, m). no mass spec

### Intermediate 114b

### tert-Butyl 4-[3-bromo-2-(prop-1-yn-1-yl)anilino]piperidine-1-carboxylate

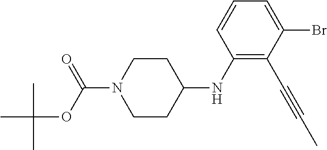 [SMILES: CC#Cc1c(Br)cccc1NC1CCN(C(=O)OC(C)(C)C)CC1]

Sodium triacetoxyborohydride (13.56 g, 63.98 mmol) was added to a solution of 3-bromo-2-(prop-1-yn-1-yl)aniline (6.72 g, 31.99 mmol) and tert-butyl 4-oxopiperidine-1-carboxylate (9.56 g, 47.98 mmol) in DCM (100 mL). AcOH (3.66 mL, 63.98 mmol) was added and the solution was stirred at 40° C. for 20 h. The reaction mixture was diluted with DCM (250 mL) and NaOH (2M, 250 mL) and the organic layer was evaporated to give the crude product. The crude product was purified by flash silica chromatography, elution gradient 0 to 30% EtOAc in heptane to give the title compound (7.09 g, 56.4%) as an oil which crystallised on standing. ¹H NMR (CDCl₃) δ 1.47 (9H, s), 1.95-2.07 (2H, m), 2.19 (4H, s), 2.98 (2H, t), 3.37-3.55 (1H, m), 3.99 (2H, s), 4.65 (1H, d), 6.51 (1H, d), 6.85 (1H, dd), 6.96 (1H, t) 1H not observed; m/z: ES⁺ [M-Boc]⁺=339.0.

### Intermediate 114c

### tert-Butyl 4-(4-bromo-2-methyl-1H-indol-1-yl)piperidine-1-carboxylate

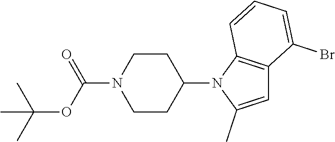 [SMILES: Cc1cc2c(Br)cccc2n1C1CCN(C(=O)OC(C)(C)C)CC1]

Sodium tetrachloroaurate(III) dihydrate (0.359 g, 0.90 mmol) was added to a solution of tert-butyl 4-[3-bromo-2-(prop-1-yn-1-yl)anilino]piperidine-1-carboxylate (7.09 g, 18.03 mmol) in EtOH (100 mL) and was stirred at 60° C. for 30 min. The solvent was evaporated and the residue was partitioned between water (250 mL) and DCM (250 mL). The organic layer was evaporated to afford crude product. The crude product was purified by flash silica chromatography, elution gradient 0 to 50% EtOAc in heptane to give the title compound (3.8 g, 54%) as a white solid. ¹H NMR (CDCl₃) δ 1.54 (9H, s), 1.83 (2H, d), 2.31-2.55 (5H, m), 2.86 (2H, t), 4.19-4.49 (3H, m), 6.31 (1H, d), 6.9-6.98 (1H, m), 7.22 (1H, dd), 7.35 (1H, t); m/z: ES⁺ [M+H]⁺=395.0.

### Intermediate 114d

### tert-Butyl 4-[4-(2,4-dioxo-1,3-diazinan-1-yl)-2-methyl-1H-indol-1-yl]piperidine-1-carboxylate

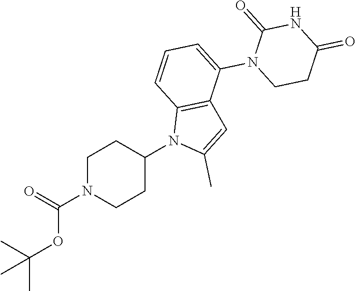 [SMILES: Cc1cc2c(N3CCC(=O)NC3=O)cccc2n1C1CCN(C(=O)OC(C)(C)C)CC1]

A suspension of tert-butyl 4-(4-bromo-2-methyl-1H-indol-1-yl)piperidine-1-carboxylate (1.18 g, 3.01 mmol), dihydropyrimidine-2,4(1H,3H)-dione (0.68 g, 6.03 mmol), tert-butylBrettPhos (0.073 g, 0.15 mmol) and Cs₂CO₃ (1.96 g, 6.03 mmol) was degassed with nitrogen. [Pd(cinnamyl)C1]₂ (0.039 g, 0.08 mmol) was then added and the reaction stirred at 90° C. for 6 h. The reaction mixture was cooled to RT and partitioned between EtOAc (20 mL) and water (20 mL). The aqueous phase was back extracted with EtOAc (10 mL) and the combined organics were washed with NaCl solution (15 mL), dried and evaporated to afford crude product. The crude product was purified by flash silica chromatography, elution gradient 0 to 100% EtOAc in heptane to give the title compound (0.409 g, 31.8%) as a brown solid. ¹H NMR δ 1.46 (9H, s), 1.80 (2H, d), 2.12-2.31 (2H, m), 2.44 (3H, s), 2.74 (2H, t), 2.96 (2H, s), 3.73 (2H, t), 4.05-4.26 (2H, m), 4.49 (1H, s), 6.16 (1H, s), 6.89 (1H, d), 6.98-7.09 (1H, m), 7.40 (1H, d), 10.29 (1H, s); m/z: ES⁺ [M+H]⁺=427.3.

### Intermediate 114e

### 1-[2-Methyl-1-(piperidin-4-yl)-1H-indol-4-yl]-1,3-diazinane-2,4-dione tosylate

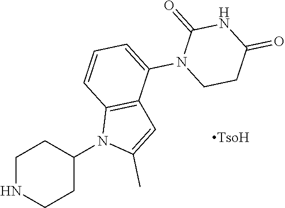 [SMILES: Cc1cc2c(N3CCC(=O)NC3=O)cccc2n1C1CCNCC1]

To a suspension of tert-butyl 4-[4-(2,4-dioxo-1,3-diazinan-1-yl)-2-methyl-1H-indol-1-yl]piperidine-1-carboxylate (1.70 g, 3.99 mmol) in MeCN (25 mL) was added 4-methylbenzenesulfonic acid hydrate (0.910 g, 4.78 mmol) and was stirred at 70° C. for 2 h. The reaction was cooled to RT, MTBE (25.00 ml) was added and the suspension was filtered and dried to give the title compound (1.86 g, 94%) as a grey solid (TsOH salt). ¹H NMR δ 1.95 (2H, d), 2.29 (3H, s), 2.45 (3H, s), 2.57-2.72 (2H, m), 2.74 (2H, t), 3.15 (2H, q), 3.49 (2H, d), 3.73 (2H, t), 4.61 (1H, t), 6.18 (1H, s), 6.93 (1H, d), 7.02-7.19 (3H, m), 7.43-7.53 (2H, m), 7.64 (1H, d), 8.24-8.65 (2H, m), 10.30 (1H, s); m/z: ES⁺ [M+H]⁺=327.4.

### Example 114

### 4-Chloro-7-[(3S)-3-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl]-1H-indole-3-carbonitrile

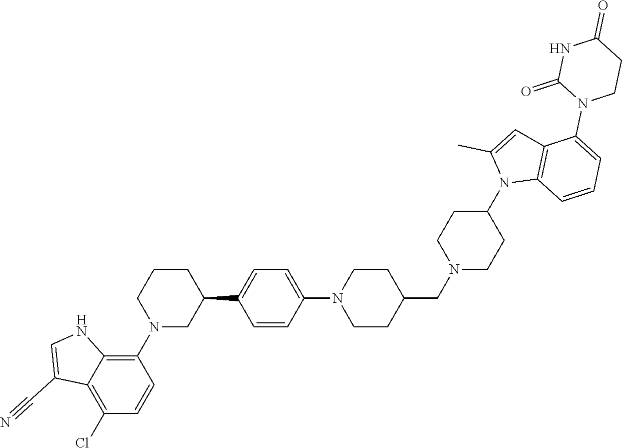 [SMILES: Cc1cc2c(N3CCC(=O)NC3=O)cccc2n1C1CCN(CC2CCN(c3ccc([C@@H]4CCCN(c5ccc(Cl)c6c(C#N)c[nH]c56)C4)cc3)CC2)CC1]

Intermediate 11c (100 mg, 0.18 mmol) was stirred in formic acid (2 mL) at 60° C. for 2 h. The solvent was evaporated and diluted with NMP (2 mL). 1-[2-Methyl-1-(piperidin-4-yl)-1H-indol-4-yl]-1,3-diazinane-2,4-dione (69.8 mg, 0.21 mmol) was added and stirred at RT for 16 h. The reaction was purified by flash C18-flash chromatography, elution gradient 0 to 60% MeCN in water (0.1% formic acid). The product was basified with NaHCO₃ solution, the MeCN was evaporated with the resulting solid collected by filtration and dried under vacuum to give the title compound (47.5 mg, 35.2%) as a white solid. ¹H NMR δ 0.83 (1H, s), 1.21-1.26 (3H, m), 1.59-1.71 (2H, m), 1.73-1.98 (7H, m), 2.14 (2H, t), 2.24 (2H, d), 2.44 (4H, s), 2.57-2.71 (4H, m), 2.74 (2H, t), 3.02 (3H, d), 3.33-3.4 (1H, m), 3.64 (2H, d), 3.74 (2H, t), 4.24 (1H, t), 6.16 (1H, s), 6.8-6.98 (4H, m), 7.05 (1H, t), 7.1-7.2 (3H, m), 7.47 (1H, d), 8.31 (1H, s), 10.30 (1H, s), 12.28 (1H, s); m/z: ES+ [M+H]⁺=757.4.

### Example 115

### Intermediate 115a

### tert-Butyl 6′-fluoro-5,6-dihydro[3,3′-bipyridine]-1(4H)-carboxylate

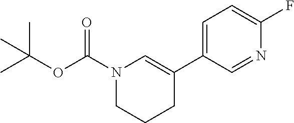 [SMILES: CC(C)(C)OC(=O)N1C=C(c2ccc(F)nc2)CCC1]

The title compound was prepared using methodology described in intermediate 107b using tert-butyl 4-(4,4,5,5-tetramethyl-1,3,2-dioxaborolan-2-yl)-3,6-dihydropyridine-1(2H)-carboxylate and 5-bromo-2-fluoropyridine to give the title compound (1.1 g, 87%) as a yellow solid. ¹H NMR (CDCl₃) δ 1.51 (9H, s), 2.34 (2H, q), 3.57 (2H, t), 4.24 (2H, q), 6.21 (1H, tt), 6.92 (1H, dd), 7.7-7.87 (1H, m), 8.16-8.31 (1H, m); m/z: ES⁺ [M+H]⁺=279.1.

### Intermediate 115b

### 6′-Fluoro-1,4,5,6-tetrahydro-3,3′-bipyridine

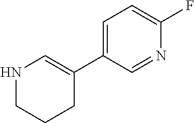 [SMILES: Fc1ccc(C2=CNCCC2)cn1]

tert-Butyl 6′-fluoro-5,6-dihydro[3,3′-bipyridine]-1(4H)-carboxylate (1.1 g, 3.95 mmol) was added to HCl (4M in 1,4-dioxane, 19.76 ml, 79.04 mmol) under nitrogen and was stirred at RT for 16 h. The reaction mixture was filtered through Celite® and the solvent was evaporated to give the title compound in the form of an HCl salt (1.0 g, 118%) as a white solid and was used without further purification. ¹H NMR δ 2.48 (2H, d), 3.17 (2H, dt), 3.97 (2H, s), 6.34-6.56 (1H, m), 7.21 (1H, dd), 8.10 (1H, td), 8.32 (1H, d), 9.89 (1H, s); m/z: ES⁺ [M+H]⁺=178.8.

### Intermediate 115c

### Benzyl 6′-fluoro-5,6-dihydro[3,3′-bipyridine]-1(4H)-carboxylate

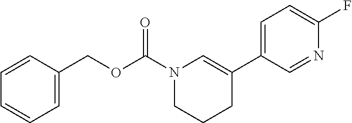 [SMILES: O=C(OCc1ccccc1)N1C=C(c2ccc(F)nc2)CCC1]

The title compound was prepared using methodology described in intermediate 32a using 6′-fluoro-1,4,5,6-tetrahydro-3,3′-bipyridine and benzyl carbonochloridate to give the title compound (1.0 g, 86%) as a yellow gum. ¹H NMR (CDCl₃) δ 2.37 (2H, s), 3.67 (2H, t), 4.33 (2H, s), 5.21 (2H, s), 6.15-6.27 (1H, m), 6.93 (1H, dd), 7.3-7.43 (5H, m), 7.77 (1H, s), 8.23 (1H, s); m z; ES⁺ [M+H]⁺=313.1.

### Intermediate 115d

### Benzyl 6′-[4-(dibutoxymethyl)piperidin-1-yl]-5,6-dihydro[3,3′-bipyridine]-1(4H)-carboxylate

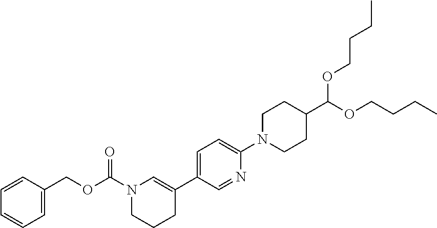 [SMILES: CCCCOC(OCCCC)C1CCN(c2ccc(C3=CN(C(=O)OCc4ccccc4)CCC3)cn2)CC1]

DIPEA (1.51 mL, 8.64 mmol) was added to benzyl 6′-fluoro-5,6-dihydro[3,3′-bipyridine]-1(4H)-carboxylate (0.9 g, 2.88 mmol) and 4-(dibutoxymethyl)piperidine (0.84 g, 3.46 mmol) in NMP (10 mL) under nitrogen. The mixture was stirred at 110° C. for 16 h. The reaction was cooled to RT and diluted with EtOAc (50 mL), washed with water (25 mL), NaCl solution (25 mL), dried over Na₂SO₄, filtered and evaporated to afford crude product. The crude product was purified by flash silica chromatography, elution gradient 0 to 30% EtOAc in Et₂O to give the title compound (0.7 g, 45.3%) as a colourless gum. ¹H NMR (CDCl₃) δ 0.94 (6H, t), 1.33-1.46 (6H, m), 1.53-1.63 (4H, m), 1.89 (3H, d), 2.33 (2H, s), 2.86 (2H, s), 3.45 (2H, dt), 3.59-3.68 (4H, m), 4.18 (1H, d), 4.33 (4H, d), 5.20 (2H, s), 6.09 (1H, s), 6.67 (1H, d), 7.3-7.44 (5H, m), 7.51 (1H, s), 8.20 (1H, d); m/z: ES⁺ [M+H]⁺=536.2.

### Intermediate 115e

### 2-[4-(Dibutoxymethyl)piperidin-1-yl]-5-(piperidin-3-yl)pyridine

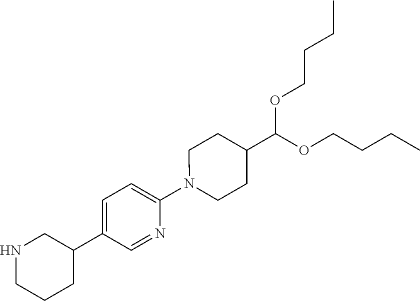 [SMILES: CCCCOC(OCCCC)C1CCN(c2ccc(C3CCCNC3)cn2)CC1]

The title compound was prepared using methodology described in intermediate 63c using benzyl 6′-[4-(dibutoxymethyl)piperidin-1-yl]-5,6-dihydro[3,3′-bipyridine]-1(4H)-carboxylate to give the title compound (0.4 g, 82%) as a colourless gum. ¹H NMR (CDCl₃) δ 0.94 (6H, t), 1.25-1.48 (7H, m), 1.53-1.62 (5H, m), 1.67-1.83 (2H, m), 1.84-1.9 (3H, m), 1.96 (2H, d), 2.54-2.71 (2H, m), 2.76 (2H, td), 2.85-3.05 (1H, m), 3.11 (1H, t), 3.45 (2H, dt), 3.63 (2H, dt), 4.17 (1H, d), 4.27 (2H, d), 6.62 (1H, dd), 7.3-7.39 (1H, m), 8.07 (1H, dd); m/z: ES⁺ [M+H]⁺=404.2.

### Intermediates 115f and 116a

The title compounds were prepared using methodology described in intermediate 11c using 2-[4-(dibutoxymethyl)piperidin-1-yl]-5-(piperidin-3-yl)pyridine and intermediate 1b to give a racemic mixture. The enantiomers were separated by preparative chiral-HPLC on a Column: CHIRALPAK IE-3, 4.6*50 mm 3 um; Mobile Phase A: Hex:DCM, 3:1)(0.1% DEA), Mobile Phase B: EtOH; Flow rate: 1 mL/min; Gradient: 30-70% B in 10.5 mins to give, in order of elution, intermediate 115f (isomer 1, 45 mg, 9%, >99% ee) and intermediate 116a (isomer 2, 45 mg, 9%, >99% ee) as white solids.

### Intermediate 115f

### 4-Chloro-7-[(3S*)-3-{6-[4-(dibutoxymethyl)piperidin-1-yl]pyridin-3-yl}piperidin-1-yl]-1H-indole-3-carbonitrile [Absolute Stereochemistry not Yet Confirmed]

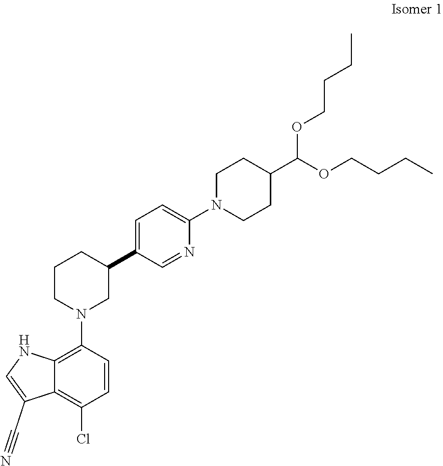 [SMILES: CCCCOC(OCCCC)C1CCN(c2ccc([C@@H]3CCCN(c4ccc(Cl)c5c(C#N)c[nH]c45)C3)cn2)CC1]

m/z: ES⁺ [M+H]⁺=578.3

### Intermediate 116a

### 4-Chloro-7-[(3R*)-3-{6-[4-(dibutoxymethyl)piperidin-1-yl]pyridin-3-yl}piperidin-1-yl]-1H-indole-3-carbonitrile [Absolute Stereochemistry not Yet Confirmed]

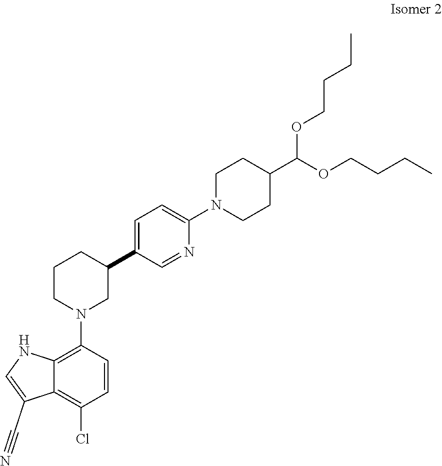 [SMILES: CCCCOC(OCCCC)C1CCN(c2ccc([C@@H]3CCCN(c4ccc(Cl)c5c(C#N)c[nH]c45)C3)cn2)CC1]

¹H NMR (CDCl₃) δ 1.39 (7H, dq), 1.58 (7H, d), 2.02 (6H, d), 3.14 (4H, d), 3.34-3.55 (4H, m), 3.58-3.69 (2H, m), 3.75 (5H, d), 4.20 (1H, d), 4.35 (2H, s), 6.87 (2H, d), 7.14 (1H, d), 7.73 (1H, s), 7.91 (1H, s) 1H not observed; m/z: ES⁺ [M+H]⁺=578.2

### Example 115

### 4-Chloro-7-[(3S*)-3-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl]-1H-indole-3-carbonitrile [Absolute Stereochemistry not Yet Confirmed]

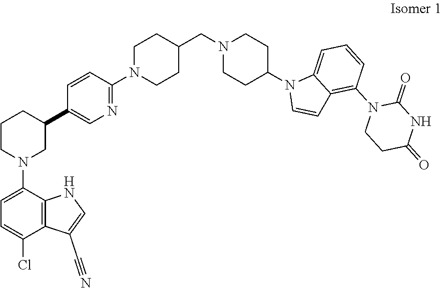

The title compound was prepared using methodology described in example 23 using intermediate 115f and intermediate 5f to give the title compound in the form of a formate salt (10 mg, 22%) as a white solid. ¹H NMR δ 0.99-1.16 (2H, m), 1.51-1.63 (1H, m), 1.7-1.83 (3H, m), 1.84-2.1 (7H, m), 2.1-2.27 (4H, m), 2.59-2.82 (6H, m), 2.99 (3H, d), 3.35-3.44 (2H, m), 3.78 (2H, t), 4.23 (2H, d), 4.3-4.42 (1H, m), 6.41 (1H, d), 6.79 (2H, dd), 6.96 (1H, d), 7.05-7.23 (2H, m), 7.43-7.59 (3H, m), 8.08 (1H, s), 8.28 (1H, s), 10.32 (1H, s), 12.27 (1H, s); m/z: ES⁺ [M+H]⁺=744.8.

### Example 116

### 4-Chloro-7-[(3R*)-3-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl]-1H-indole-3-carbonitrile [Absolute Stereochemistry not Yet Confirmed]

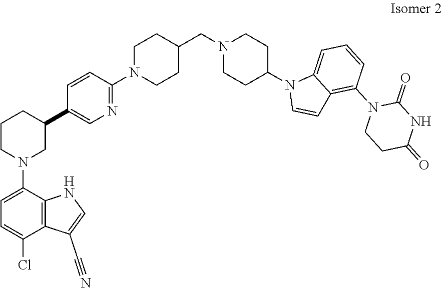

The title compound was prepared using methodology described in example 23 using intermediate 116a and intermediate 5f to give the title compound in the form of a formate salt (10 mg, 22%) as a white solid. ¹H NMR δ 1.01-1.17 (2H, m), 1.56 (1H, s), 1.67-2.1 (10H, m), 2.1-2.35 (4H, m), 2.56-2.69 (2H, m), 2.69-2.84 (4H, m), 2.86-3.07 (3H, m), 3.78 (4H, t), 4.24 (2H, d), 4.38 (1H, d), 6.41 (1H, d), 6.56 (1H, s), 6.76 (1H, d), 6.93 (2H, dd), 7.14 (1H, t), 7.4-7.6 (3H, m), 7.97-8.13 (2H, m), 10.32 (1H, s) 1H not observed; m/z: ES⁺ [M+H]⁺=744.3.

### Example 117

### Intermediate 117a

### Benzyl 2-(4-bromophenyl)morpholine-4-carboxylate

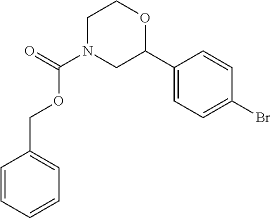 [SMILES: O=C(OCc1ccccc1)N1CCOC(c2ccc(Br)cc2)C1]

The title compound was prepared using methodology described in intermediate 32a using 2-(4-bromophenyl)morpholine and benzyl carbonochloridate to give the title compound (5.0 g, 73%) as a white solid. ¹H NMR (CDCl3) δ 1.86 (2H, d), 2.67 (1H, tt), 2.90 (2H, t), 4.36 (2H, d), 5.18 (2H, s), 7.1-7.25 (2H, m), 7.32-7.44 (7H, m); m/z: ES⁺ [M+H]⁺=376.1.

### Intermediate 117b

### Benzyl 2-{4-[4-(dibutoxymethyl)piperidin-1-yl]phenyl}morpholine-4-carboxylate

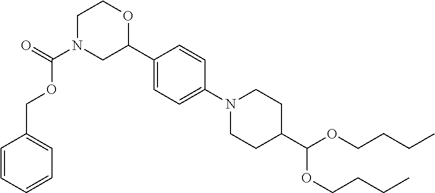 [SMILES: CCCCOC(OCCCC)C1CCN(c2ccc(C3CN(C(=O)OCc4ccccc4)CCO3)cc2)CC1]

The title compound was prepared using methodology described in intermediate 11a using benzyl 2-(4-bromophenyl)morpholine-4-carboxylate and 4-(dibutoxymethyl)piperidine to give the title compound (1.8 g, 25%) as a yellow oil. ¹H NMR (CDCl₃) δ 0.95 (6H, t), 1.36-1.52 (5H, m), 1.53-1.73 (6H, m), 1.87 (5H, t), 2.56-2.77 (3H, m), 2.89 (2H, s), 3.47 (2H, dt), 3.59-3.79 (3H, m), 4.16-4.49 (3H, m), 5.18 (2H, s), 6.61-6.87 (2H, m), 7.12-7.25 (1H, m), 7.29-7.51 (6H, m); m/z: ES⁺ [M+H]⁺=539.1.

### Intermediate 117c

### 2-{4-[4-(Dibutoxymethyl)piperidin-1-yl]phenyl}morpholine

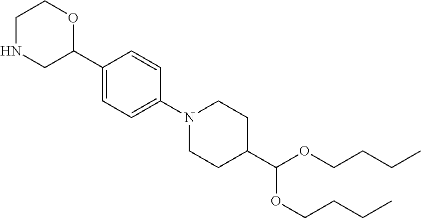 [SMILES: CCCCOC(OCCCC)C1CCN(c2ccc(C3CNCCO3)cc2)CC1]

The title compound was prepared using methodology described in intermediate 63c using benzyl 2-{4-[4-(dibutoxymethyl)piperidin-1-yl]phenyl}morpholine-4-carboxylate to give the title compound (1.2 g, 89%) as a yellow oil. ¹H NMR (CDCl₃) δ 0.94 (6H, td), 1.36-1.48 (5H, m), 1.53-1.64 (4H, m), 1.67-1.92 (3H, m), 2.66 (2H, tt), 2.75-3.16 (4H, m), 3.37-3.53 (4H, m), 3.55-3.87 (5H, m), 3.92-4.05 (1H, m), 4.19 (1H, dd), 4.42 (1H, dt), 6.92 (2H, dd), 7.24 (2H, dd); m/z: ES⁺ [M+H]⁺=405.2.

### Intermediate 117d and 118a

The title compounds were prepared using methodology described in intermediate 11c using 2-{4-[4-(dibutoxymethyl)piperidin-1-yl]phenyl}morpholine and intermediate 1b to give a racemic mixture. The enantiomers were separated by preparative chiral-HPLC on a Column: CHIRALPAK IA-3, 4.6*50 mm, 3 μm; Flow rate: 1 m/min; Gradient: 0% B; Mobile Phase A: Hex:DCM (3:1, 0.1% DEA), Mobile Phase B: MeCN; to give intermediate 117d (isomer 1, 120 mg, 14%, >99% ee) and intermediate 118a (isomer 2, 140 mg, 16%, >99% ee) as white solids.

### Intermediate 117d

### 4-Chloro-7-[(2S*)-2-{4-[4-(dibutoxymethyl)piperidin-1-yl]phenyl}morpholin-4-yl]-1H-indole-3-carbonitrile [Absolute Stereochemistry not Yet Confirmed]

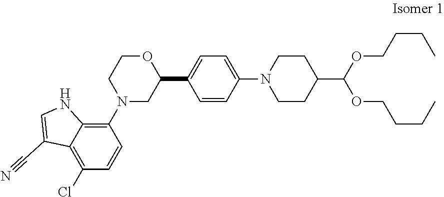

¹H NMR δ (CDCl₃) δ 0.95 (6H, t), 1.16-1.31 (2H, m), 1.41 (4H, h), 1.58 (6H, q), 2.88 (3H, s), 3.15 (2H, s), 3.33 (1H, s), 3.4-3.57 (2H, m), 3.57-3.85 (5H, m), 4.08 (1H, t), 4.21 (2H, d), 4.82 (1H, s), 6.89 (2H, d), 7.18 (2H, d), 7.40 (2H, s), 7.78 (1H, s) 1H not observed; m/z: ES⁺ [M+H]⁺=579.0.

### Intermediate 118a

### 4-Chloro-7-[(2R*)-2-{4-[4-(dibutoxymethyl)piperidin-1-yl]phenyl}morpholin-4-yl]-1H-indole-3-carbonitrile [Absolute Stereochemistry not Yet Confirmed]

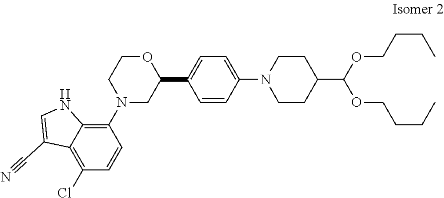

¹H NMR (CDCl₃) δ 0.95 (6H, t), 1.2-1.49 (8H, m), 1.59 (7H, t), 2.89 (4H, s), 3.15 (2H, s), 3.46 (3H, q), 3.69 (4H, dd), 4.08 (1H, s), 4.21 (2H, d), 6.89 (2H, d), 7.18 (2H, d), 7.78 (1H, s) 1H not observed; m/z: ES⁺ [M+H]⁺ =579.0.

### Example 117

### 4-Chloro-7-[(2S*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indole-3-carbonitrile [Absolute Stereochemistry not Yet Confirmed]

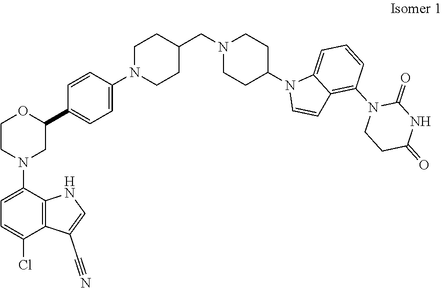

Intermediate 117d (100 mg, 0.17 mmol) was stirred in formic acid (2 mL) at 40° C. for 1 h. The mixture was then cooled to RT and evaporated to dryness. The residue was added to intermediate 5f (84 mg, 0.17 mmol) and sodium acetate (170 mg, 0.21 mmol) in NMP (1 mL) which was stirred at 40° C. for 2 h. The reaction mixture was purified by flash C18-flash chromatography, elution gradient 0 to 60% MeCN in water (0.1% formic acid). The fractions containing product were combined and adjusted to pH 8 NaHCO₃ solution. The solid was filtered and dried in a vacuum oven to give the title compound (20 mg, 15%) as a white solid. ¹H NMR δ 1.22 (3H, d), 1.68 (1H, s), 1.82 (2H, d), 1.88-2.09 (4H, m), 2.1-2.32 (4H, m), 2.57-2.7 (3H, m), 2.76 (2H, t), 2.88 (1H, t), 2.99 (2H, d), 3.26 (1H, s), 3.68 (2H, d), 3.78 (2H, t), 3.9-4.16 (2H, m), 4.37 (1H, s), 4.71 (1H, d), 6.41 (1H, d), 6.82 (1H, d), 6.93 (3H, dd), 7.14 (2H, t), 7.27 (2H, d), 7.43-7.57 (2H, m), 8.34 (1H, s), 10.32 (1H, s), 12.45 (1H, s); m/z: ES⁺ [M+H]⁺=745.3.

### Example 118

### 4-Chloro-7-[(2R*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indole-3-carbonitrile

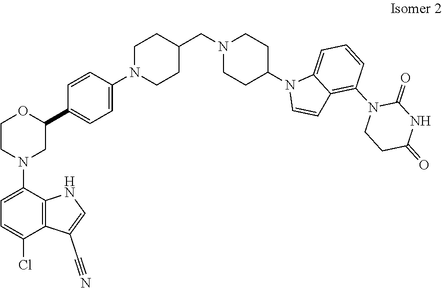

The title compound was prepared using methodology described in example 117 using intermediate 118a to give the title compound (20 mg, 15%) as a white solid. ¹H NMR δ 1.22 (2H, q), 1.68 (1H, s), 1.77-1.88 (2H, m), 1.89-2.06 (4H, m), 2.16 (2H, t), 2.22-2.3 (2H, m), 2.58-2.71 (3H, m), 2.76 (2H, t), 2.84 (1H, t), 2.99 (2H, d), 3.53 (1H, s), 3.68 (3H, d), 3.78 (2H, t), 3.93 (1H, t), 4-4.09 (1H, m), 4.29-4.46 (1H, m), 4.6-4.73 (1H, m), 6.41 (1H, d), 6.63 (1H, d), 6.87-7.02 (4H, m), 7.13 (1H, q), 7.25 (2H, d), 7.45-7.6 (2H, m), 8.14 (1H, d), 10.32 (1H, s) 1H not observed; m/z: ES⁺ [M+H]⁺=745.3.

### Intermediate 119a and 120a

The title compounds were prepared using methodology described in intermediate 11c using intermediate 117c and intermediate 9c to give a racemic mixture. The enantiomers were separated by preparative chiral-HPLC on a Column: CHIRAL ART Cellulose-SB, 2*25 cm, 5 μm; Flow rate: 20 m/min; Gradient: 20% B; Mobile Phase A: Hex (0.5% 2M NH₃-MeOH), Mobile Phase B: EtOH; to give, in order of elution, intermediate 119a (isomer 1, 110 mg, 12%, >99% ee) and intermediate 120a (isomer 2, 120 mg, 14%, >99% ee) as yellow solids.

### Intermediate 119a

### 4-Chloro-7-[(2S*)-2-{4-[4-(dibutoxymethyl)piperidin-1-yl]phenyl}morpholin-4-yl]-1H-indazole-3-carbonitrile [Absolute Stereochemistry not Yet Confirmed]

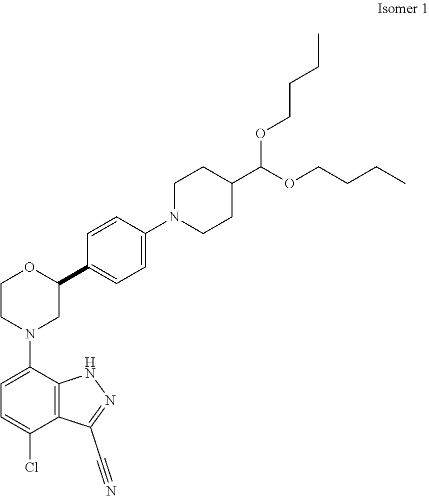 [SMILES: CCCCOC(OCCCC)C1CCN(c2ccc([C@H]3CN(c4ccc(Cl)c5c(C#N)n[nH]c45)CCO3)cc2)CC1]

¹H NMR (CDCl₃) δ 0.94 (6H, t), 1.42 (6H, p), 1.58 (5H, t), 1.85 (4H, d), 2.70 (2H, d), 2.96 (1H, s), 3.14 (1H, d), 3.26 (1H, s), 3.45 (2H, td), 3.65 (2H, q), 3.73 (2H, d), 4.10 (1H, t), 4.23 (2H, d), 4.75 (1H, s), 6.93 (3H, s), 7.21 (1H, d), 7.28 (2H, s); m/z: ES⁺ [M+H]⁺=580.2.

### Intermediate 120a

### 4-Chloro-7-[(2R*)-2-{4-[4-(dibutoxymethyl)piperidin-1-yl]phenyl}morpholin-4-yl]-1H-indazole-3-carbonitrile [Absolute Stereochemistry not Yet Confirmed]

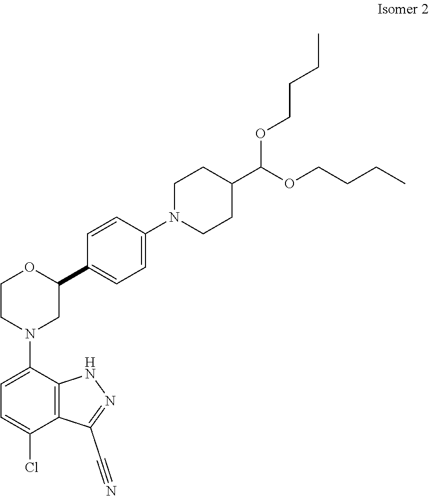 [SMILES: CCCCOC(OCCCC)C1CCN(c2ccc([C@H]3CN(c4ccc(Cl)c5c(C#N)n[nH]c45)CCO3)cc2)CC1]

¹H NMR (CDCl₃) δ 0.94 (6H, t), 1.35-1.49 (5H, m), 1.53-1.63 (5H, m), 1.83 (3H, d), 2.70 (2H, d), 2.96 (1H, s), 3.12 (1H, t), 3.27 (1H, d), 3.35 (1H, s), 3.46 (2H, dt), 3.64 (2H, dt), 3.73 (2H, d), 4.09 (1H, t), 4.22 (2H, d), 4.74 (1H, s), 6.91 (3H, d), 7.22 (1H, d), 7.28 (2H, s), 11.02 (1H, s); m/z: ES⁺ [M+H]⁺=579.4.

### Example 119

### 4-Chloro-7-[(2S*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indazole-3-carbonitrile [Absolute Stereochemistry not Yet Confirmed]

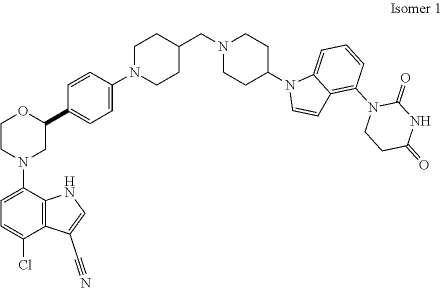

The title compound was prepared using methodology described in example 117 using intermediate 119a and intermediate 5f to give the title compound (6 mg, 5%) as a white solid. ¹H NMR δ 1.16-1.32 (2H, m), 1.70 (1H, s), 1.83 (2H, d), 1.91-2.11 (4H, m), 2.11-2.4 (4H, m), 2.59-2.73 (3H, m), 2.76 (2H, t), 2.85-2.98 (1H, m), 3.03 (2H, d), 3.41 (1H, d), 3.49 (1H, d), 3.69 (2H, d), 3.78 (2H, t), 3.91-4.04 (1H, m), 4.08 (1H, d), 4.39 (1H, s), 4.6-4.84 (1H, m), 6.42 (1H, d), 6.8-7.06 (4H, m), 7.15 (1H, t), 7.28 (3H, dd), 7.45-7.62 (2H, m), 10.32 (1H, s) 1H not observed; m/z: ES⁺ [M+H]⁺=746.3.

### Example 120

### 4-Chloro-7-[(2R*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indazole-3-carbonitrile

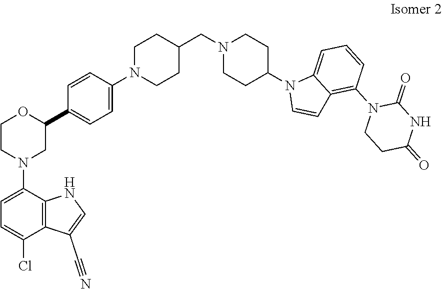

The title compound was prepared using methodology described in example 117 using intermediate 120a and intermediate 5f to give the title compound (49 mg, 38%) as a white solid. ¹H NMR δ 1.13-1.29 (2H, m), 1.69 (1H, s), 1.82 (2H, d), 1.88-2.11 (4H, m), 2.13-2.32 (4H, m), 2.56-2.72 (3H, m), 2.76 (2H, t), 2.87-2.97 (1H, m), 3.01 (2H, d), 3.52 (2H, d), 3.69 (2H, d), 3.78 (2H, t), 3.9-4.03 (1H, m), 4.08 (1H, d), 4.39 (1H, s), 4.70 (1H, d), 6.42 (1H, d), 6.79-7.02 (4H, m), 7.14 (1H, t), 7.26 (3H, dd), 7.45-7.62 (2H, m), 10.34 (1H, s) 1H not observed; m/z: ES⁺ [M+H]⁺=746.3.

### Example 121

### Intermediate 121a

### tert-Butyl 5-(6-chloropyridazin-3-yl)-3,4-dihydropyridine-1(2H)-carboxylate

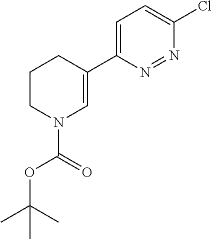 [SMILES: CC(C)(C)OC(=O)N1C=C(c2ccc(Cl)nn2)CCC1]

The title compound was prepared using methodology described in intermediate 107b using tert-butyl 4-(4,4,5,5-tetramethyl-1,3,2-dioxaborolan-2-yl)-3,6-dihydropyridine-1(2H)-carboxylate and 3-bromo-6-chloropyridazine to give the title compound (1.2 g, 62%) as a white solid. ¹H NMR δ 2.47-2.58 (4H, m), 3.18 (5H, s), 3.34 (2H, s), 4.09 (2H, s), 5.81 (2H, s), 7.96-8.58 (2H, m), 8.53 (1H, d); m/z: ES⁺ [M+H]⁺=296.2.

### Intermediate 121b

### 3-Chloro-6-(1,4,5,6-tetrahydropyridin-3-yl)pyridazine

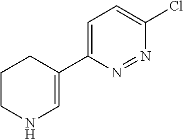 [SMILES: Clc1ccc(C2=CNCCC2)nn1]

The title compound was prepared using methodology described in intermediate 75f using tert-butyl 5-(6-chloropyridazin-3-yl)-3,4-dihydropyridine-1(2H)-carboxylate to give the title compound (650 mg, 82%) as a white solid. ¹H NMR δ 1.36 (3H, s), 2.52 (2H, s), 3.58 (2H, s), 6.98 (1H, d), 7.93-8.04 (1H, m), 8.22 (1H, d); m/z: ES⁺ [M+H]⁺=196.0.

### Intermediate 121c

### Benzyl 5-(6-chloropyridazin-3-yl)-3,4-dihydropyridine-1(2H)-carboxylate

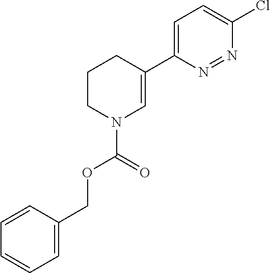 [SMILES: O=C(OCc1ccccc1)N1C=C(c2ccc(Cl)nn2)CCC1]

The title compound was prepared using methodology described in intermediate 32a using 3-chloro-6-(1,4,5,6-tetrahydropyridin-3-yl)pyridazine and benzyl carbonochloridate to give the title compound (610 mg, 66%) as a white solid. ¹H NMR δ 2.38 (2H, dp), 3.59 (2H, s), 4.51 (2H, s), 5.14 (2H, s), 6.95-7.02 (1H, m), 7.25-7.45 (5H, m), 7.88 (1H, d), 8.12 (1H, d); m/z: ES⁺ [M+H]⁺=330.1.

### Intermediate 121d

### Benzyl 5-{6-[4-(dibutoxymethyl)piperidin-1-yl]pyridazin-3-yl}-3,4-dihydropyridine-1(2H)-carboxylate

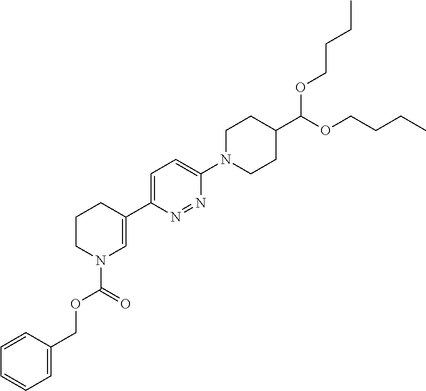 [SMILES: CCCCOC(OCCCC)C1CCN(c2ccc(C3=CN(C(=O)OCc4ccccc4)CCC3)nn2)CC1]

The title compound was prepared using methodology described in intermediate 63a using benzyl 5-{6-[4-(dibutoxymethyl)piperidin-1-yl]pyridazin-3-yl}-3,4-dihydropyridine-1(2H)-carboxylate and 4-(dibutoxymethyl)piperidine to give the title compound (690 mg, 71%) as a yellow gum. ¹H NMR δ 0.88 (6H, t), 1.17 (1H, t), 1.23 (2H, s), 1.33 (4H, dp), 1.48 (4H, dq), 1.72 (2H, d), 1.86 (1H, s), 1.99 (1H, s), 2.78-2.9 (2H, m), 3.38 (2H, dt), 3.55 (3H, dt), 4.03 (1H, q), 4.17 (1H, d), 4.39 (2H, d), 4.47 (2H, d), 5.13 (2H, s), 6.60 (1H, s), 7.25 (1H, d), 7.28-7.42 (5H, m), 7.69 (1H, d); m/z: ES⁺ [M+H]⁺=537.2.

### Intermediate 121e

### 3-[4-(Dibutoxymethyl)piperidin-1-yl]-6-(piperidin-3-yl)pyridazine

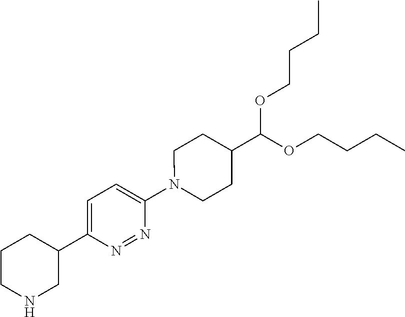 [SMILES: CCCCOC(OCCCC)C1CCN(c2ccc(C3CCCNC3)nn2)CC1]

The title compound was prepared using methodology described in intermediate 63c using benzyl 5-{6-[4-(dibutoxymethyl)piperidin-1-yl]pyridazin-3-yl}-3,4-dihydropyridine-1(2H)-carboxylate to give the title compound (500 mg, 96%) as a yellow gum. ¹H NMR δ 1.17 (4H, t), 1.23 (2H, s), 1.34 (4H, m), 1.48 (4H, dq), 1.59-1.81 (3H, m), 1.99 (4H, s), 2.06-2.35 (OH, m), 2.53 (3H, s), 2.67 (3H, s), 2.64-2.93 (3H, m), 3.55 (1H, d), 4.03 (3H, q), 4.17 (1H, d), 4.32 (2H, d), 5.11 (OH, d), 7.20 (1H, d), 7.33 (2H, dd); m/z: ES⁺ [M+H]⁺=405.2.

### Intermediates 121f and intermediate 122a

The title compound was prepared using methodology described in intermediate 11c using 3-[4-(dibutoxymethyl)piperidin-1-yl]-6-(piperidin-3-yl)pyridazine and intermediate 1b to give the title compound as a racemic mixture. The enantiomers were separated by preparative chiral-HPLC on a Column: CHIRALPAK IG-3, 4.6*50 mm, 3 μm; Flow rate: 1 mL/min; Mobile Phase A: Hex:DCM (3:1, 0.1% DEA), Mobile Phase B: EtOH; to give, in order of elution, intermediate 121f (isomer 1, 70 mg, 9%, >99% ee) and intermediate 122a (isomer 2, 70 mg, 9%, >99% ee) as yellow solids.

### Intermediate 121f

### 4-Chloro-7-[(3R*)-3-{6-[4-(dibutoxymethyl)piperidin-1-yl]pyridazin-3-yl}piperidin-1-yl]-1H-indole-3-carbonitrile [Absolute Stereochemistry not Yet Confirmed]

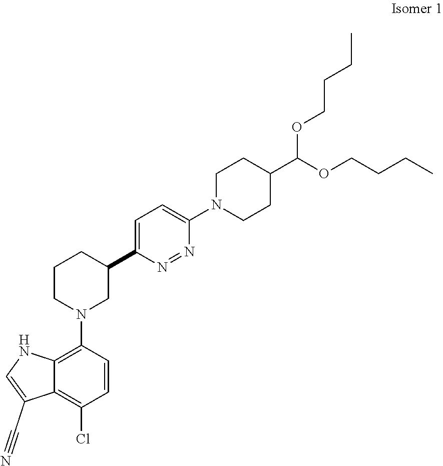 [SMILES: CCCCOC(OCCCC)C1CCN(c2ccc([C@@H]3CCCN(c4ccc(Cl)c5c(C#N)c[nH]c45)C3)nn2)CC1]

¹H NMR δ 0.88 (6H, t), 1.24 (4H, s), 1.27-1.42 (3H, m), 1.49 (4H, p), 1.74 (3H, d), 1.86 (4H, s), 2.03 (1H, s), 2.83 (3H, t), 3.27 (2H, s), 3.30 (1H, s), 3.34-3.46 (1H, m), 3.56 (2H, dt), 4.18 (1H, d), 4.38 (2H, d), 6.83 (1H, d), 7.15 (1H, d), 7.29 (1H, d), 7.47 (1H, d), 8.35 (1H, d), 12.94 (1H, s); m/z: ES⁺ [M+H]⁺=579.2.

### Intermediate 122a

### 4-Chloro-7-[(3S*)-3-{6-[4-(dibutoxymethyl)piperidin-1-yl]pyridazin-3-yl}piperidin-1-yl]-1H-indole-3-carbonitrile [Absolute Stereochemistry not Yet Confirmed]

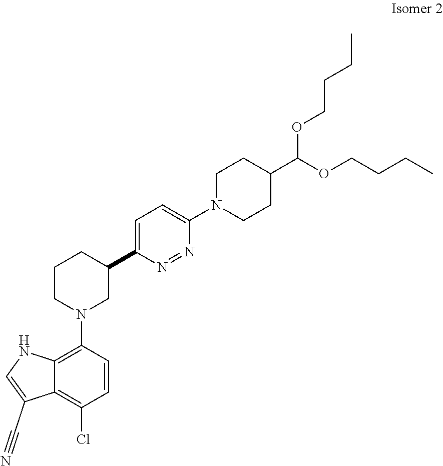 [SMILES: CCCCOC(OCCCC)C1CCN(c2ccc([C@@H]3CCCN(c4ccc(Cl)c5c(C#N)c[nH]c45)C3)nn2)CC1]

¹H NMR δ 0.88 (6H, t), 1.19-1.32 (4H, m), 1.33 (1H, d), 1.35 (1H, s), 1.33-1.42 (1H, m), 1.42-1.56 (4H, m), 1.72 (2H, s), 1.86 (4H, s), 2.82 (1H, s), 3.16-3.69 (12H, m), 4.18 (1H, d), 6.82 (1H, d), 7.13 (1H, d), 7.28 (1H, d), 7.47 (1H, d), 8.34 (1H, s), 12.93 (1H, s); m/z: ES⁺ [M+H]⁺=579.2.

### Example 121

### 4-Chloro-7-[(3R*)-3-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl]-1H-indole-3-carbonitrile [Absolute Stereochemistry not Yet Confirmed]

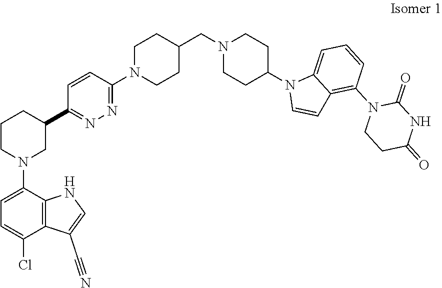

The title compound was prepared using methodology described in example 23 using intermediate 121f and intermediate 5f to give the title compound in the form of a formate salt (33 mg, 36%) as a white solid. ¹H NMR δ 1.18 (3H, t), 1.73 (1H, s), 1.86 (5H, s), 2.02 (5H, d), 2.28 (4H, s), 2.3-2.36 (2H, m), 2.76 (3H, t), 2.81-2.95 (1H, m), 3.07 (3H, s), 3.27 (1H, s), 3.78 (2H, t), 4.37 (3H, d), 6.43 (1H, d), 6.83 (1H, d), 6.97 (1H, d), 7.15 (2H, t), 7.30 (1H, d), 7.39-7.57 (3H, m), 8.35 (1H, d), 10.32 (1H, s), 12.93 (1H, s); m/z: ES⁺ [M+H]*=745.4.

### Example 122

### 4-Chloro-7-[(3S*)-3-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl]-1H-indole-3-carbonitrile

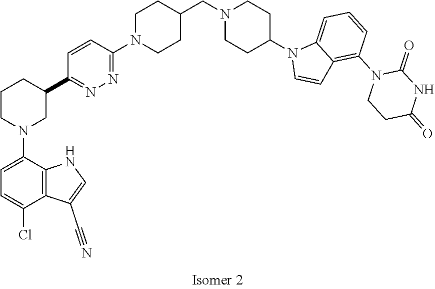 [SMILES: N#Cc1c[nH]c2c(N3CCC[C@@H](c4ccc(N5CCC(CN6CCC(n7ccc8c(N9CCC(=O)NC9=O)cccc87)CC6)CC5)nn4)C3)ccc(Cl)c12]

The title compound was prepared using methodology described in example 23 using intermediate 122a and intermediate 5f to give the title compound in the form of a formate salt (24 mg, 26%) as a white solid. ¹H NMR δ 1.06-1.2 (2H, m), 1.82 (1H, s), 1.85 (5H, s), 1.9-2.07 (5H, m), 2.12-2.21 (4H, m), 2.23 (2H, d), 2.87 (3H, q), 3.00 (2H, d), 3.28 (3H, s), 3.35 (1H, s), 3.78 (2H, t), 4.37 (3H, dd), 6.42 (1H, d), 6.81 (1H, d), 6.96 (1H, d), 7.09-7.19 (2H, m), 7.29 (1H, d), 7.42-7.61 (3H, m), 8.33 (1H, s), 10.32 (1H, s), 12.97 (1H, s); m/z: ES⁺ [M+H]⁺=745.4.

### Example 123

### 4-Chloro-7-(4-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl)-1H-indole-3-carbonitrile

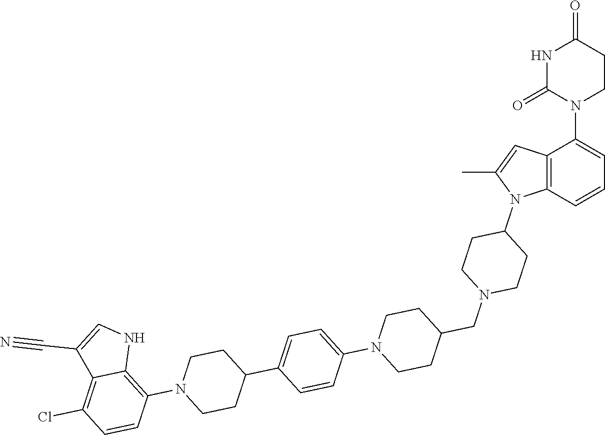 [SMILES: Cc1cc2c(N3CCC(=O)NC3=O)cccc2n1C1CCN(CC2CCN(c3ccc(C4CCN(c5ccc(Cl)c6c(C#N)c[nH]c56)CC4)cc3)CC2)CC1]

Intermediate 87c (103 mg, 0.18 mmol) was added to formic acid (2 mL) and stirred at 60° C. for 2 h. The solvent was evaporated and dissolved in NMP (2 mL). Intermediate 114e was added after the reaction was stirred for 16 h. Purification of the reaction mixture by preparative HPLC (Column A, Eluent A) gave the title compound in the form of a formate salt (16 mg, 12%) as a white solid. ¹H NMR δ 1.22-1.3 (3H, m), 1.73-2.01 (9H, m), 2.14 (2H, t), 2.26 (2H, d), 2.45 (4H, s), 2.6-2.65 (3H, m), 2.71-2.82 (4H, m), 3.03 (2H, d), 3.46 (2H, d), 3.67 (2H, d), 3.74 (2H, t), 4.25 (1H, t), 6.16 (1H, s), 6.79-7 (4H, m), 7.03-7.21 (4H, m), 7.48 (1H, d), 8.29 (1H, s), 10.30 (1H, s), 12.28 (1H, s); m/z: ES⁺ [M+H]⁺=757.3.

### Example 124

### Intermediate 124a

### Benzyl 4-(2-fluoropyrimidin-5-yl)-3,6-dihydropyridine-1(2H)-carboxylate

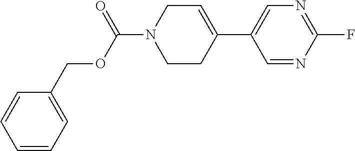 [SMILES: O=C(OCc1ccccc1)N1CC=C(c2cnc(F)nc2)CC1]

The title compound was prepared using methodology described in intermediate 63b using 4-(4,4,5,5-tetramethyl-1,3,2-dioxaborolan-2-yl)-3,6-dihydropyridine-1(2H)-carboxylate and 5-bromo-2-fluoropyrimidine to give the title compound (2.5 g, 91%) as a yellow gum. ¹H NMR δ 2.55 (2H, s), 3.32 (1H, s), 4.12 (3H, s), 5.13 (2H, s), 6.40 (1H, s), 7.27-7.43 (5H, m), 8.88 (2H, d); m/z: ES⁺ [M+H]⁺=314.0.

### Intermediate 124b

### Benzyl 4-{2-[4-(dibutoxymethyl)piperidin-1-yl]pyrimidin-5-yl}-3,6-dihydropyridine-1(2H)-carboxylate

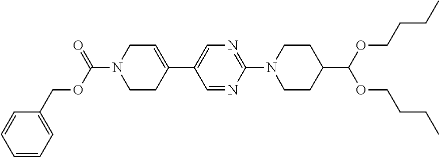 [SMILES: CCCCOC(OCCCC)C1CCN(c2ncc(C3=CCN(C(=O)OCc4ccccc4)CC3)cn2)CC1]

The title compound was prepared using methodology described in intermediate 63a using benzyl 4-(2-fluoropyrimidin-5-yl)-3,6-dihydropyridine-1(2H)-carboxylate and 4-(dibutoxymethyl)piperidine to give the title compound (3.5 g, 85%) as a white solid. ¹H NMR δ 0.88 (6H, t), 1.08-1.23 (2H, m), 1.24-1.41 (4H, m), 1.41-1.55 (4H, m), 1.71 (2H, d), 1.83 (1H, s), 2.44 (2H, s), 2.82 (2H, t), 3.29-3.45 (3H, m), 3.58 (3H, dd), 4.06 (2H, s), 4.16 (1H, d), 4.67 (2H, d), 5.12 (2H, s), 6.07 (1H, s), 7.26-7.42 (5H, m), 8.44 (2H, s); m/z: ES⁺ [M+H]⁺=537.2.

### Intermediate 124c

### 2-[4-(Dibutoxymethyl)piperidin-1-yl]-5-(piperidin-4-yl)pyrimidine

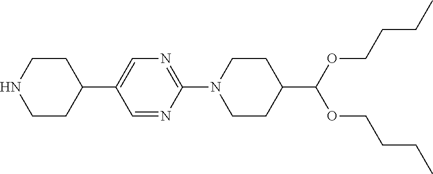 [SMILES: CCCCOC(OCCCC)C1CCN(c2ncc(C3CCNCC3)cn2)CC1]

The title compound was prepared using methodology described in intermediate 63c using benzyl 4-{2-[4-(dibutoxymethyl)piperidin-1-yl]pyrimidin-5-yl}-3,6-dihydropyridine-1(2H)-carboxylate to give the title compound (2.5 g, 95%) as a colourless gum. ¹H NMR δ 0.88 (6H, t), 1.25-1.41 (4H, m), 1.48 (6H, p), 1.62 (1H, s), 1.69 (3H, d), 2.77 (2H, t), 3.01 (2H, d), 3.25-3.39 (5H, m), 3.55 (2H, dt), 4.10 (2H, s), 4.16 (1H, d), 4.35 (2H, s), 4.63 (2H, d), 8.22 (2H, s); m/z: ES⁺ [M+H]⁺=405.2.

### Intermediate 124d

### 4-Chloro-7-(4-{2-[4-(dibutoxymethyl)piperidin-1-yl]pyrimidin-5-yl}piperidin-1-yl)-1H-indole-3-carbonitrile

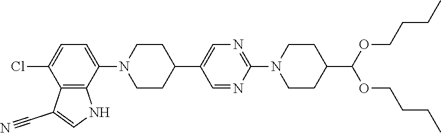 [SMILES: CCCCOC(OCCCC)C1CCN(c2ncc(C3CCN(c4ccc(Cl)c5c(C#N)c[nH]c45)CC3)cn2)CC1]

The title compound was prepared using methodology described in intermediate 63d using 2-[4-(dibutoxymethyl)piperidin-1-yl]-5-(piperidin-4-yl)pyrimidine and intermediate 1b to give the title compound (600 mg, 42%) as a pale yellow gum. ¹H NMR δ (84° C.) δ 0.88 (6H, t), 1.07-1.25 (2H, m), 1.33 (4H, dp), 1.48 (4H, dq), 1.71 (2H, d), 1.76-2.02 (4H, m), 2.54 (1H, d), 2.71-2.84 (4H, m), 3.32 (1H, s), 3.35-3.46 (4H, m), 3.55 (2H, dt), 4.17 (1H, d), 4.65 (2H, d), 6.86 (1H, d), 7.16 (1H, d), 8.31 (3H, d), 12.28 (1H, s); m/z: ES⁺ [M+H]⁺=579.4.

### Example 124

### 4-Chloro-7-(4-{2-[4-({4-[2-(2,6-dioxopiperidin-3-yl)-1-oxo-2,3-dihydro-1H-isoindol-5-yl]piperazin-1-yl}methyl)piperidin-1-yl]pyrimidin-5-yl}piperidin-1-yl)-1H-indole-3-carbonitrile

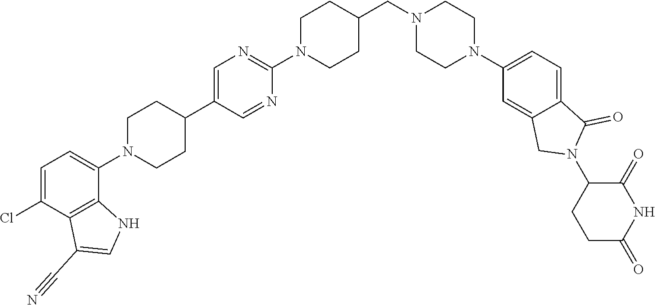 [SMILES: N#Cc1c[nH]c2c(N3CCC(c4cnc(N5CCC(CN6CCN(c7ccc8c(c7)CN(C7CCC(=O)NC7=O)C8=O)CC6)CC5)nc4)CC3)ccc(Cl)c12]

Intermediate 1i (144 mg, 0.34 mmol) was stirred in formic acid (1 mL) at 40° C. for 1 h. 4-Chloro-7-(4-{2-[4-(dibutoxymethyl)piperidin-1-yl]pyrimidin-5-yl}piperidin-1-yl)-1H-indole-3-carbonitrile (150 mg, 0.26 mmol) was also stirred in formic acid (1 mL) and stirred at 60° C. for 2 h. The two mixtures were evaporated to dryness, combined with NMP (2 mL) and stirred at RT for 16 h. The reaction mixture purified by flash C18-flash chromatography, elution gradient 0 to 40% MeCN in water (0.1% formic acid). The fractions containing product were combined and basified with NaHCO₃ solution. The resulting solid was filtered under vacuum and dried to give the title compound (30 mg, 15%) as a white solid. ¹H NMR δ 1.05 (2H, q), 1.84 (5H, dt), 1.91-2.03 (3H, m), 2.20 (2H, d), 2.36 (1H, tt), 2.51-2.63 (3H, m), 2.72-2.98 (5H, m), 3.29 (4H, t), 3.35 (3H, s), 3.42 (2H, d), 4.21 (1H, d), 4.33 (1H, d), 4.64 (2H, d), 5.06 (1H, dd), 6.86 (1H, d), 6.99-7.29 (3H, m), 7.53 (1H, d), 8.32 (3H, d), 10.97 (1H, s), 12.31 (1H, s); m/z: ES⁺ [M+H]⁺=761.4.

### Example 125

### 4-Chloro-7-(4-{2-[4-({4-[4-(2,4-dioxo-1,3-diazinan-1-yl)-1H-indol-1-yl]piperidin-1-yl}methyl)piperidin-1-yl]pyrimidin-5-yl}piperidin-1-yl)-1H-indole-3-carbonitrile

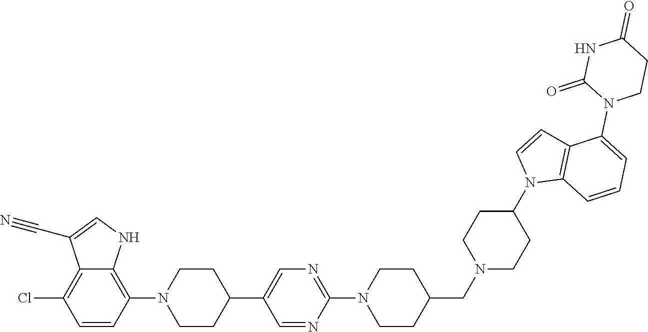 [SMILES: N#Cc1c[nH]c2c(N3CCC(c4cnc(N5CCC(CN6CCC(n7ccc8c(N9CCC(=O)NC9=O)cccc87)CC6)CC5)nc4)CC3)ccc(Cl)c12]

The title compound was prepared using methodology described in example 63 using intermediate 124d and intermediate 5f to give the title compound (30 mg, 15%) as a white solid. ¹H NMR δ 1.05 (2H, d), 1.83 (5H, t), 1.97 (6H, q), 2.17 (2H, s), 2.22 (2H, d), 2.55 (1H, d), 2.77 (4H, q), 2.86 (2H, t), 3.00 (2H, d), 3.42 (2H, d), 3.78 (2H, t), 4.37 (1H, s), 4.65 (2H, d), 6.42 (1H, d), 6.91 (2H, dd), 7.15 (2H, dd), 7.47-7.57 (2H, m), 8.31 (3H, d), 10.33 (1H, s), 12.29 (1H, s); m/z: ES⁺ [M+H]⁺=745.5.

### Example 126

### Intermediate 126a

### Benzyl (2S)-2-(hydroxymethyl)morpholine-4-carboxylate

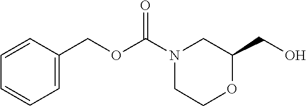 [SMILES: O=C(OCc1ccccc1)N1CCO[C@H](CO)C1]

HCl (1.25M in MeOH, 200 mL, 800.0 mmol) was added to tert-butyl (2S)-2-(hydroxymethyl)morpholine-4-carboxylate (15 g, 69.04 mmol) in MeOH (50 mL) at RT for 2 h. The solvent was evaporated to afford a colourless gum and was diluted in THF:water (1:1, 300 mL). Benzyl carbonochloridate (11.8 mL, 82.85 mmol) was added followed by Na₂CO₃ (21.95 g, 207.12 mmol) and the solution was stirred at RT for 16 h. The mixture was diluted with EtOAc (200 mL), washed with water (100 mL), NaCl solution (100 mL), dried over Na₂SO₄, filtered and evaporated to afford crude product. The crude product was purified by flash silica chromatography, elution gradient 0 to 50% EtOAc in Et₂O to give the title compound (15.4 g, 89%) as a colourless gum. ¹H NMR (CDCl₃) δ 2.86 (1H, t), 3.03 (1H, t), 3.46-3.73 (4H, m), 3.84-4.09 (3H, m), 5.17 (2H, d), 7.3-7.44 (5H, m) 1H not observed; m/z: ES⁺ [M+H]⁺=252.2.

### Intermediate 126b

### Benzyl (2S)-2-formylmorpholine-4-carboxylate

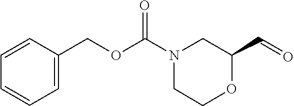 [SMILES: O=C[C@@H]1CN(C(=O)OCc2ccccc2)CCO1]

Oxalyl chloride (5.30 mL, 62.08 mmol) was added to DMSO (9.0 mL) and DCM (150 mL) at −78° C. under nitrogen. Benzyl (2S)-2-(hydroxymethyl)morpholine-4-carboxylate (13.0 g, 51.73 mmol) was added to the mixture followed by triethylamine (36.1 mL, 258.67 mmol). The solution was stirred at RT for 2 h and was then diluted with DCM (150 mL), washed with water (125 mL), NaCl solution (125 mL), dried over Na₂SO₄, filtered and evaporated to afford crude product. The crude product was purified by flash silica chromatography, elution gradient 0 to 60% EtOAc in Et₂O to give the title compound (10.4 g, 81%) as a colourless gum. ¹H NMR (CDCl₃) δ 2.72-3.24 (2H, m), 3.32-3.68 (2H, m), 3.89 (3H, dq), 5.06-5.18 (2H, m), 7.18-7.41 (5H, m), 9.59 (1H, s); m/z: ES⁺ [M+H]⁺=249.8.

### Intermediate 126c

### Benzyl (2S)-2-(dibutoxymethyl)morpholine-4-carboxylate

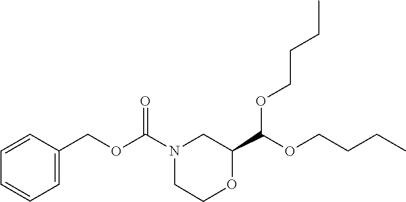 [SMILES: CCCCOC(OCCCC)[C@@H]1CN(C(=O)OCc2ccccc2)CCO1]

4-Methylbenzene-1-sulfonic acid (1.52 g, 8.02 mmol) was added to benzyl (2S)-2-formylmorpholine-4-carboxylate (10.0 g, 40.12 mmol) in 1-butanol (100 mL) at RT and was stirred at 90° C. for 2 h. The reaction was cooled to RT and the solvent was evaporated to afford crude product. The crude product was purified by flash silica chromatography, elution gradient 0 to 50% EtOAc in Et₂O to give the title compound (10.5 g, 69.0%) as a colourless oil. ¹H NMR (CDCl₃) δ 0.93 (6H, td), 1.31-1.5 (4H, m), 1.5-1.67 (4H, m), 2.86 (1H, s), 2.95-3.11 (1H, m), 3.37-3.76 (6H, m), 3.78-4.02 (2H, m), 4.13 (1H, dd), 4.41 (1H, d), 5.17 (2H, s), 7.29-7.43 (5H, m); m/z: ES⁺ [M+Na]=402.3.

### Intermediate 126d

### (2S)-2-(Dibutoxymethyl)morpholine

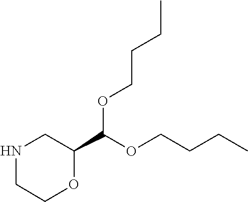 [SMILES: CCCCOC(OCCCC)[C@@H]1CNCCO1]

The title compound was prepared using methodology described in intermediate 41c using benzyl (2S)-2-(dibutoxymethyl)morpholine-4-carboxylate to give the title compound (3.0 g, 93%) as a colourless oil and was used without further purification. ¹H NMR δ 0.88 (6H, t), 1.24-1.4 (4H, m), 1.41-1.55 (4H, m), 2.34-2.46 (1H, m), 2.56-2.68 (2H, m), 2.73-2.81 (1H, m), 3.25-3.49 (5H, m), 3.51-3.62 (2H, m), 3.69-3.75 (1H, m), 4.27 (1H, d). no mass ion

### Intermediate 126e

### Benzyl 4-{4-[(2S)-2-(dibutoxymethyl)morpholin-4-yl]phenyl}piperidine-1-carboxylate

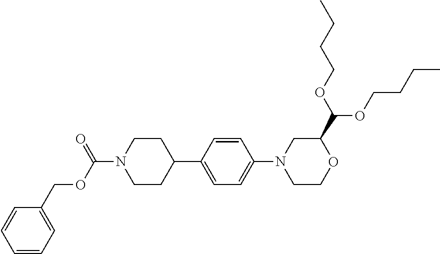 [SMILES: CCCCOC(OCCCC)[C@@H]1CN(c2ccc(C3CCN(C(=O)OCc4ccccc4)CC3)cc2)CCO1]

The title compound was prepared using methodology described in intermediate 11a using (2S)-2-(dibutoxymethyl)morpholine and intermediate 1c to give the title compound (2.8 g, 44%) as a yellow oil. ¹H NMR δ 0.89 (6H, m), 1.18 (1H, t), 1.33-1.52 (8H, m), 1.75 (1H, d), 1.99 (1H, s), 2.35-2.7 (4H, m), 3.26-3.72 (9H, m), 3.85-4.21 (4H, m), 4.44 (1H, d), 5.10 (2H, s), 6.72-6.91 (2H, m), 6.97-7.18 (2H, m), 7.27-7.5 (5H, m); m/z: ES⁺ [M+H]⁺=539.1.

### Intermediate 126f

### (2S)-2-(Dibutoxymethyl)-4-[4-(piperidin-4-yl)phenyl]morpholine

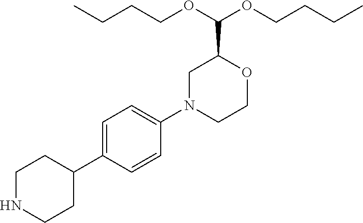 [SMILES: CCCCOC(OCCCC)[C@@H]1CN(c2ccc(C3CCNCC3)cc2)CCO1]

The title compound was prepared using methodology described in intermediate 11b using benzyl 4-{4-[(2S)-2-(dibutoxymethyl)morpholin-4-yl]phenyl}piperidine-1-carboxylate to give the title compound (1.9 g, 90%) as a brown oil. ¹H NMR δ 0.8-1.01 (6H, m), 1.18 (1H, t), 1.31-1.44 (5H, m), 1.45-1.54 (4H, m), 1.58-1.71 (2H, m), 1.99 (1H, s), 2.45-2.65 (5H, m), 3.00 (2H, d), 3.16-3.7 (8H, m), 3.75-4.23 (1H, m), 4.44 (1H, d), 6.71-6.92 (2H, m), 6.99-7.17 (2H, m); m/z: ES⁺ [M+H]⁺=405.1.

### Intermediate 126g

### 7-(4-{4-[(2S)-2-(Dibutoxymethyl)morpholin-4-yl]phenyl}piperidin-1-yl)-4-fluoro-1H-indole-3-carbonitrile

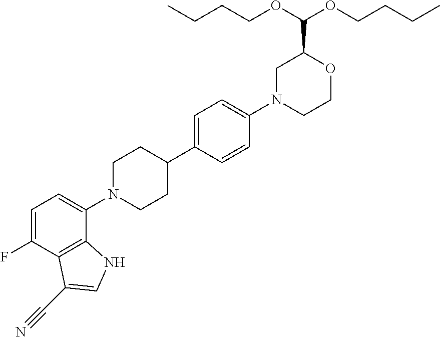 [SMILES: CCCCOC(OCCCC)[C@@H]1CN(c2ccc(C3CCN(c4ccc(F)c5c(C#N)c[nH]c45)CC3)cc2)CCO1]

The title compound was prepared using methodology described in intermediate 63d using (2S)-2-(dibutoxymethyl)-4-[4-(piperidin-4-yl)phenyl]morpholine and intermediate 2b to give the title compound (280 mg, 67%) as a brown foam. ¹H NMR δ 0.86-0.95 (6H, m), 1.29-1.41 (4H, m), 1.45-1.57 (4H, m), 1.78-1.88 (2H, m), 1.9-2.04 (2H, m), 2.51-2.71 (3H, m), 2.71-2.83 (2H, m), 3.34-3.42 (2H, m), 3.42-3.55 (4H, m), 3.55-3.69 (4H, m), 3.9-4 (1H, m), 4.45 (1H, d), 6.82 (1H, dd), 6.86-6.98 (3H, m), 7.17 (2H, d), 8.26 (1H, s), 12.24 (1H, s); m/z: ES⁺ [M+H]⁺=563.3.

### Example 126

### 7-(4-{4-[(2R)-2-({4-[2-(2,6-dioxopiperidin-3-yl)-1-oxo-2,3-dihydro-1H-isoindol-5-yl]piperazin-1-yl}methyl)morpholin-4-yl]phenyl}piperidin-1-yl)-4-fluoro-1H-indole-3-carbonitrile

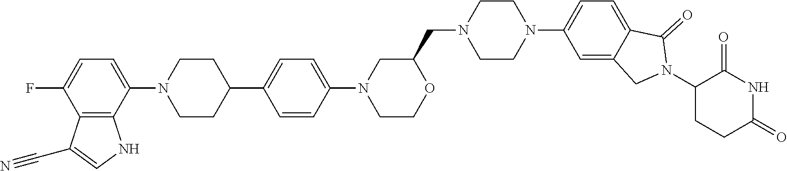 [SMILES: N#Cc1c[nH]c2c(N3CCC(c4ccc(N5CCO[C@H](CN6CCN(c7ccc8c(c7)CN(C7CCC(=O)NC7=O)C8=O)CC6)C5)cc4)CC3)ccc(F)c12]

HCl (2M aq, 2 ml, 4.00 mmol) was added to 7-(4-{4-[(2S)-2-(dibutoxymethyl)morpholin-4-yl]phenyl}piperidin-1-yl)-4-fluoro-1H-indole-3-carbonitrile (100 mg, 0.18 mmol) in THF (2 mL). The mixture was stirred at 60° C. for 24 h and then basified using NaOH (2M, 2 mL). The suspension was extracted with EtOAc (4 mL) and the layers were separated. The organic layer was dried over MgSO₄, filtered and evaporated to afford a residue. Separately, intermediate 1i (76 mg, 0.18 mmol) was stirred in formic acid (2 mL) at 40° C. for 1 h. The solvent was evaporated to afford a residue. The two residues were combined in NMP (2 mL) and stirred at 40° C. for 2 h. Purification of the reaction mixture by preparative HPLC (Column A, Eluent A) gave the title compound in the form of a formate salt (28 mg, 21%) as a white solid. ¹H NMR δ 1.84 (2H, d), 1.89-2.04 (3H, m), 2.29-2.46 (2H, m), 2.49 (2H, s), 2.51 (1H, s), 2.53-2.7 (7H, m), 2.71-2.81 (2H, m), 2.83-2.97 (1H, m), 3.29 (3H, s), 3.37 (2H, d), 3.46 (1H, d), 3.53-3.61 (1H, m), 3.61-3.72 (1H, m), 3.77 (1H, d), 3.9-3.98 (1H, m), 4.13-4.38 (2H, m), 5.05 (1H, dd), 6.82 (1H, dd), 6.89-6.98 (3H, m), 7.06 (2H, d), 7.17 (2H, d), 7.52 (1H, d), 8.26 (1H, s), 10.94 (1H, s), 12.24 (1H, s); m/z: ES⁺ [M+H]⁺=745.3.

### Example 127

### Intermediate 127a

### 4-Chloro-7-(4-{4-[(2S)-2-(dibutoxymethyl)morpholin-4-yl]phenyl}piperidin-1-yl)-1H-indole-3-carbonitrile

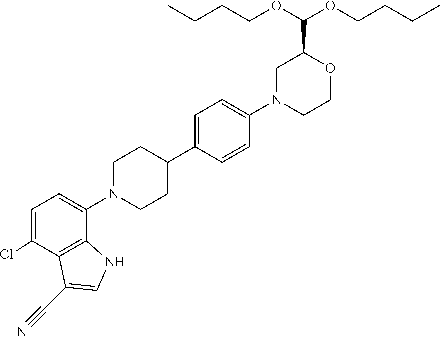 [SMILES: CCCCOC(OCCCC)[C@@H]1CN(c2ccc(C3CCN(c4ccc(Cl)c5c(C#N)c[nH]c45)CC3)cc2)CCO1]

The title compound was prepared using methodology described in intermediate 63d using intermediate 126f and intermediate 1b to give the title compound (230 mg, 53%) as an orange solid. ¹H NMR δ 0.86-0.91 (6H, m), 1.16 (1H, t), 1.23 (1H, s), 1.3-1.39 (4H, m), 1.50 (4H, q), 1.85 (2H, s), 2.77 (1H, s), 3.31-3.76 (14H, m), 3.92-4.02 (1H, m), 4.44 (1H, d), 6.8-6.9 (3H, m), 7.12-7.19 (3H, m), 8.31 (1H, d), 12.26 (1H, s); m/z: ES⁺ [M+H]⁺=579.0.

### Intermediate 127b

### 4-Chloro-7-(4-{4-[(2S)-2-formylmorpholin-4-yl]phenyl}piperidin-1-yl)-1H-indole-3-carbonitrile

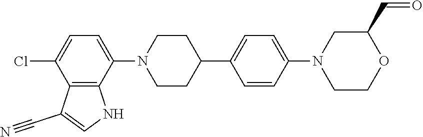 [SMILES: N#Cc1c[nH]c2c(N3CCC(c4ccc(N5CCO[C@H](C=O)C5)cc4)CC3)ccc(Cl)c12]

HCl (2M aq, 5.18 mL, 10.36 mmol) was added to 4-chloro-7-(4-{4-[(2S)-2-(dibutoxymethyl)morpholin-4-yl]phenyl}piperidin-1-yl)-1H-indole-3-carbonitrile (200 mg, 0.35 mmol) in THF (2 mL). The mixture was stirred at 60° C. for 12 h and then cooled to RT. The solution was basified using NaOH (2M, 2 ml) and the aqueous layer was extracted with EtOAc (100 mL). The organic layer was evaporated to dryness to give the title compound (120 mg, 77%) as a brown solid and was used in the next step without further purification. ¹H NMR δ 0.85 (1H, d), 1.06 (1H, t), 1.25 (5H, s), 1.58 (2H, d), 1.77 (1H, s), 1.90 (3H, s), 2.76 (3H, d), 5.33 (1H, s), 6.85-6.98 (2H, m), 7.19 (1H, d), 7.27 (1H, d), 8.15 (3H, s), 9.64 (1H, s). no mass ion

### Example 127

### 4-Chloro-7-(4-{4-[(2R)-2-({4-[2-(2,6-dioxopiperidin-3-yl)-1-oxo-2,3-dihydro-1H-isoindol-5-yl]piperazin-1-yl}methyl)morpholin-4-yl]phenyl}piperidin-1-yl)-1H-indole-3-carbonitrile

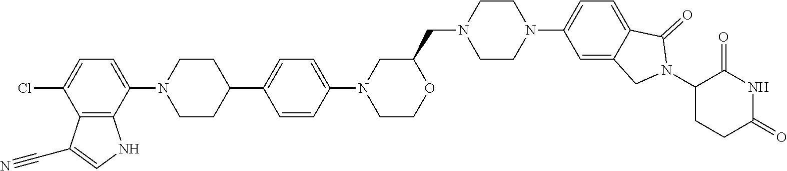 [SMILES: N#Cc1c[nH]c2c(N3CCC(c4ccc(N5CCO[C@H](CN6CCN(c7ccc8c(c7)CN(C7CCC(=O)NC7=O)C8=O)CC6)C5)cc4)CC3)ccc(Cl)c12]

The title compound was prepared using methodology described in example 23 using 4-chloro-7-(4-{4-[(2S)-2-formylmorpholin-4-yl]phenyl}piperidin-1-yl)-1H-indole-3-carbonitrile and intermediate 1i to give the title compound in the form of a formate salt (16 mg, 8%) as a white solid. ¹H NMR δ 1.84 (2H, d), 1.9-2.04 (3H, m), 2.28-2.47 (2H, m), 2.50 (2H, s), 2.52 (1H, s), 2.54-2.71 (7H, m), 2.71-2.83 (2H, m), 2.84-2.97 (1H, m), 3.29 (1H, s), 3.32 (1H, s), 3.37-3.52 (4H, m), 3.58 (1H, d), 3.62-3.71 (1H, m), 3.77 (1H, s), 3.9-3.99 (1H, m), 4.21 (1H, d), 4.34 (1H, d), 4.98-5.1 (1H, m), 6.86 (1H, d), 6.93 (2H, d), 7.07 (2H, d), 7.14-7.2 (3H, m), 7.53 (1H, d), 8.33 (1H, s), 10.96 (1H, s) 1H not observed; m/z: ES⁺ [M+H]⁺=761.4.

### Example 128

### Intermediate 128a

### tert-Butyl 7-methylidene-2-azaspiro[3.5]nonane-2-carboxylate

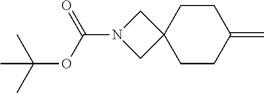 [SMILES: C=C1CCC2(CC1)CN(C(=O)OC(C)(C)C)C2]

Potassium tert-butoxide (3.4 g, 30.29 mmol) was added to methyltriphenylphosphonium bromide (10.8 g, 30.29 mmol) in THF (30 mL) at 0° C. under nitrogen. The mixture was stirred at RT for 1 h followed by the addition of tert-butyl 7-oxo-2-azaspiro[3.5]nonane-2-carboxylate (2.9 g, 12.12 mmol) and was stirred at 35° C. for 1 h. The reaction mixture was diluted with EtOAc (125 mL), washed with NH₄Cl solution (2×75 mL), NaCl solution (75 mL), dried over Na₂SO₄, filtered and evaporated to afford crude product. The crude product was purified by flash silica chromatography, elution gradient 0 to 4% EtOAc in DCM to give the title compound (2.7 g, 94%) as a white solid. ¹H NMR (CDCl₃) δ 1.46 (9H, s), 1.67-1.79 (4H, m), 2.06-2.19 (4H, m), 3.64 (4H, s), 4.61-4.69 (2H, m); m/z: ES⁺ [M-tBu]=181.9.

### Intermediate 128b

### tert-Butyl 7-(hydroxymethyl)-2-azaspiro[3.5]nonane-2-carboxylate

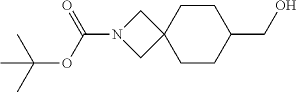 [SMILES: CC(C)(C)OC(=O)N1CC2(CCC(CO)CC2)C1]

Borane-tetrahydrofuran complex (28.4 mL, 28.44 mmol) was added to tert-butyl 7-methylidene-2-azaspiro[3.5]nonane-2-carboxylate (2.7 g, 11.38 mmol) in THF (100 mL) at 0° C. under nitrogen and stirred for 48 h. H₂O₂(5.81 mL, 56.88 mmol) and NaOH (2M, 17.06 mL, 34.13 mmol) was added and the mixture was stirred at RT for 3 h. The solvent was evaporated and diluted with DCM (100 mL). The organic layer was washed with NaCl solution (3×50 mL), dried over Na₂SO₄, filtered and evaporated to afford crude product. The crude product was purified by flash silica chromatography, elution gradient 0 to 4% THF in DCM to give the title compound (2.3 g, 79%) as a colourless oil. ¹H NMR (CDCl₃) δ 0.89-1.04 (2H, m), 1.38-1.50 (12H, m), 1.70-1.77 (2H, m), 1.85-1.98 (2H, m), 3.45 (2H, t), 3.56 (2H, s), 3.60 (2H, s), 5.27-5.34 (1H, m); m/z: ES⁺ [M+H]⁺=256.1.

### Intermediate 128c

### 2-Azaspiro[3.5]nonan-7-yl)methanol

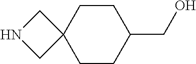 [SMILES: OCC1CCC2(CC1)CNC2]

The title compound was prepared using methodology described in intermediate 75f using tert-butyl 7-(hydroxymethyl)-2-azaspiro[3.5]nonane-2-carboxylate to give the title compound (540 mg, 76%) as a white solid. ¹H NMR δ 0.85 (2H, tdd), 1.19-1.43 (3H, m), 1.53-1.67 (2H, m), 1.91-2.07 (2H, m), 3.17 (2H, d), 3.47-3.67 (4H, m), 4.52 (1H, s), 9.39 (1H, s); m/z: ES⁺ [M+H]⁺=155.9.

### Intermediate 128d

### Benzyl 7-(hydroxymethyl)-2-azaspiro[3.5]nonane-2-carboxylate

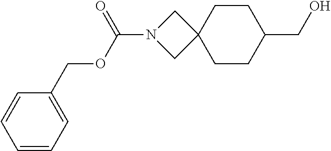 [SMILES: O=C(OCc1ccccc1)N1CC2(CCC(CO)CC2)C1]

The title compound was prepared using methodology described in intermediate 113a using (2-azaspiro[3.5]nonan-7-yl)methanol and benzyl carbonochloridate to give the title compound (500 mg, 66%) as a colourless gum. ¹H NMR (CDCl₃) δ 0.89-1.05 (2H, m), 1.46 (3H, td), 1.61 (1H, s), 1.66-1.81 (2H, m), 1.86-1.98 (2H, m), 3.45 (2H, d), 3.67 (4H, d), 5.11 (2H, s), 7.28-7.43 (5H, m); m/z: ES⁺ [M+H]⁺=290.2.

### Intermediate 128e

### Benzyl 7-formyl-2-azaspiro[3.5]nonane-2-carboxylate

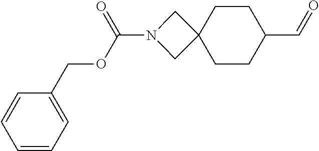 [SMILES: O=CC1CCC2(CC1)CN(C(=O)OCc1ccccc1)C2]

The title compound was prepared using methodology described in intermediate 126b using benzyl 7-(hydroxymethyl)-2-azaspiro[3.5]nonane-2-carboxylate to give the title compound (350 mg, 88%) as a colourless gum. ¹H NMR (CDCl₃) δ 1.32-1.64 (4H, m), 1.8-1.96 (4H, m), 2.22 (1H, tdt), 3.66 (4H, d), 5.09 (2H, s), 7.27-7.43 (5H, m), 9.62 (1H, d); m/z: ES⁺ [M+H]⁺=288.1.

### Intermediate 128f

### Benzyl 7-(dibutoxymethyl)-2-azaspiro[3.5]nonane-2-carboxylate

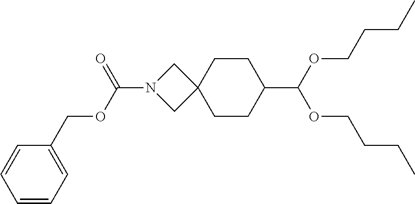 [SMILES: CCCCOC(OCCCC)C1CCC2(CC1)CN(C(=O)OCc1ccccc1)C2]

The title compound was prepared using methodology described in intermediate 126c using benzyl 7-formyl-2-azaspiro[3.5]nonane-2-carboxylate to give the title compound (5.5 g, 55%) as a colourless gum. ¹H NMR (CDCl₃) δ 0.84-1.09 (8H, m), 1.32-1.46 (6H, m), 1.48-1.64 (5H, m), 1.76 (2H, dd), 1.90 (2H, d), 3.39 (2H, dt), 3.51-3.71 (6H, m), 4.07 (1H, d), 5.09 (2H, s), 7.26-7.42 (5H, m); m/z: ES⁺ [M+H]⁺=418.4.

### Intermediate 128g

### 7-(Dibutoxymethyl)-2-azaspiro[3.5]nonane

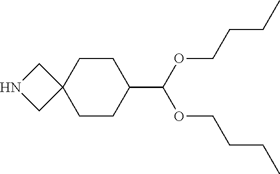 [SMILES: CCCCOC(OCCCC)C1CCC2(CC1)CNC2]

The title compound was prepared using methodology described in intermediate 60c using benzyl 7-(dibutoxymethyl)-2-azaspiro[3.5]nonane-2-carboxylate to give the title compound (3.5 g, 99%) as a yellow liquid. ¹H NMR δ 0.88 (6H, t), 0.94-1.02 (1H, m), 1.06 (1H, t), 1.19-1.54 (10H, m), 1.54-1.65 (2H, m), 1.94 (2H, d), 3.15-3.41 (5H, m), 3.39-3.57 (3H, m), 4.06 (1H, d), 4.87 (2H, s); m/z: ES⁺ [M+H]⁺=284.3.

### Intermediate 128h

### Benzyl 4-{4-[7-(dibutoxymethyl)-2-azaspiro[3.5]nonan-2-yl]phenyl}piperidine-1-carboxylate

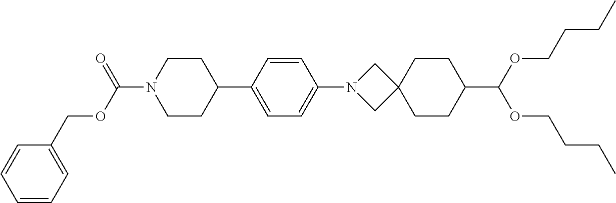 [SMILES: CCCCOC(OCCCC)C1CCC2(CC1)CN(c1ccc(C3CCN(C(=O)OCc4ccccc4)CC3)cc1)C2]

The title compound was prepared using methodology described in intermediate 11a using 7-(dibutoxymethyl)-2-azaspiro[3.5]nonane and intermediate 1c to give the title compound (1.5 g, 67%) as a yellow gum. ¹H NMR δ 0.89 (6H, t), 1.18 (1H, t), 1.43-1.59 (15H, m), 2.51 (5H, q), 3.29-3.61 (7H, m), 4.14 (5H, dd), 5.10 (4H, d), 7.25 (2H, d), 7.38 (5H, d), 7.39 (2H, t); m/z: ES⁺ [M+H]⁺=577.3.

### Intermediate 128i

### 7-(Dibutoxymethyl)-2-[4-(piperidin-4-yl)phenyl]-2-azaspiro[3.5]nonane

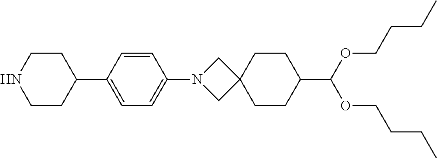 [SMILES: CCCCOC(OCCCC)C1CCC2(CC1)CN(c1ccc(C3CCNCC3)cc1)C2]

The title compound was prepared using methodology described in intermediate 60c using benzyl 4-{4-[7-(dibutoxymethyl)-2-azaspiro[3.5]nonan-2-yl]phenyl}piperidine-1-carboxylate to give the title compound (500 mg, 59%) as a yellow gum. ¹H NMR δ 0.89 (6H, t), 0.95-1.14 (2H, m), 1.21-1.57 (12H, m), 1.55-1.72 (5H, m), 1.87 (2H, d), 2.32-2.5 (1H, m), 2.5-2.65 (3H, m), 2.98 (2H, d), 3.28-3.59 (7H, m), 4.11 (1H, d), 6.23-6.44 (2H, m), 6.96-7.04 (2H, m) 1H not observed; m/z: ES⁺ [M+H]⁺=443.4.

### Intermediate 128j

### 7-(4-{4-[7-(Dibutoxymethyl)-2-azaspiro[3.5]nonan-2-yl]phenyl}piperidin-1-yl)-4-fluoro-1H-indole-3-carbonitrile

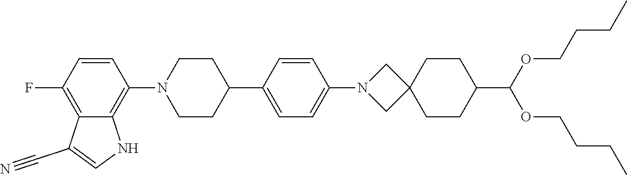 [SMILES: CCCCOC(OCCCC)C1CCC2(CC1)CN(c1ccc(C3CCN(c4ccc(F)c5c(C#N)c[nH]c45)CC3)cc1)C2]

The title compound was prepared using methodology described in intermediate 60d using 7-(dibutoxymethyl)-2-[4-(piperidin-4-yl)phenyl]-2-azaspiro[3.5]nonane and intermediate 2b to give the title compound (210 mg, 62%) as a yellow gum. ¹H NMR δ 0.90 (6H, d), 1.07 (2H, t), 1.18 (1H, t), 1.24 (1H, s), 1.28-1.39 (5H, m), 1.37-1.54 (7H, m), 1.78-1.93 (5H, m), 2.54 (1H, s), 2.75 (2H, t), 3.34-3.41 (3H, m), 3.43 (2H, s), 3.45-3.58 (4H, m), 4.04 (1H, q), 4.12 (1H, d), 6.37 (2H, dd), 6.84 (1H, d), 6.93 (1H, dd), 7.07 (2H, dd), 8.27 (1H, s), 12.25 (1H, s); m/z: ES⁺ [M+H]⁺=599.5.

### Example 128

### 7-(4-{4-[7-({4-[2-(2,6-Dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl)-4-fluoro-1H-indole-3-carbonitrile

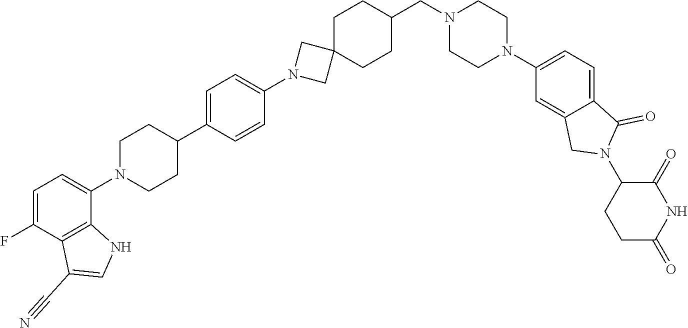 [SMILES: N#Cc1c[nH]c2c(N3CCC(c4ccc(N5CC6(CCC(CN7CCN(c8ccc9c(c8)CN(C8CCC(=O)NC8=O)C9=O)CC7)CC6)C5)cc4)CC3)ccc(F)c12]

7-(4-{4-[7-(Dibutoxymethyl)-2-azaspiro[3.5]nonan-2-yl]phenyl}piperidin-1-yl)-4-fluoro-1H-indole-3-carbonitrile (100 mg, 0.17 mmol) was stirred in formic acid (1 mL) at 60° C. for 2 h. The mixture was evaporated to dryness to afford a residue. Intermediate 1i (86 mg, 0.20 mmol) was stirred in formic acid (1 mL) at 40° C. for 1 h. The mixture was then evaporated to dryness to afford a residue. The two residues were dissolved in NMP (3 mL) and stirred at RT for 16 h. The reaction mixture was purified by flash C18-flash chromatography, elution gradient 0 to 40% MeCN in water (0.1% formic acid) to give the title compound in the form of a formate salt (18 mg, 14%) as a white solid. ¹H NMR δ 0.96 (2H, q), 1.39-1.6 (3H, m), 1.73 (2H, d), 1.78-2.02 (7H, m), 2.15 (2H, d), 2.3-2.44 (1H, m), 2.48 (4H, s), 2.52-2.63 (2H, m), 2.74 (2H, q), 2.91 (1H, m), 3.28 (4H, s), 3.37 (2H, d), 3.48 (4H, d), 4.13-4.38 (2H, m), 5.05 (1H, dd), 6.39 (2H, d), 6.82 (1H, dd), 6.93 (1H, dd), 7.03-7.13 (4H, m), 7.53 (1H, d), 8.27 (1H, d), 10.95 (1H, s), 12.25 (1H, s); m/z: ES⁺ [M+H]⁺=783.7.

### Example 129

### Intermediate 129a

### 4-Chloro-7-(4-{4-[7-(dibutoxymethyl)-2-azaspiro[3.5]nonan-2-yl]phenyl}piperidin-1-yl)-1H-indole-3-carbonitrile

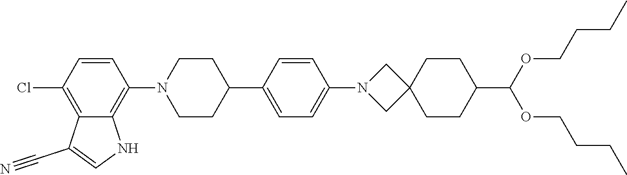 [SMILES: CCCCOC(OCCCC)C1CCC2(CC1)CN(c1ccc(C3CCN(c4ccc(Cl)c5c(C#N)c[nH]c45)CC3)cc1)C2]

The title compound was prepared using methodology described in intermediate 60d using intermediate 128i and intermediate 1b to give the title compound (500 mg, 44%) as a brown gum. ¹H NMR δ 0.78-0.93 (6H, m), 0.95-1.27 (2H, m), 1.34 (3H, h), 1.48 (4H, dt), 1.66 (2H, d), 1.79-1.89 (3H, m), 1.92 (2H, d), 1.98 (1H, d), 2.72-2.86 (2H, m), 3.33 (10H, s), 3.34-3.42 (2H, m), 3.53 (2H, dt), 4.12 (1H, d), 6.39 (2H, d), 6.87 (1H, t), 7.09 (2H, d), 7.14-7.27 (1H, m), 7.28-7.4 (1H, m), 8.3-8.37 (1H, m), 12.27 (1H, s); m/z: ES⁺ [M+H]⁺=617.0.

### Example 129

### 4-Chloro-7-(4-{4-[7-({4-[2-(2,6-dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl)-1H-indole-3-carbonitrile

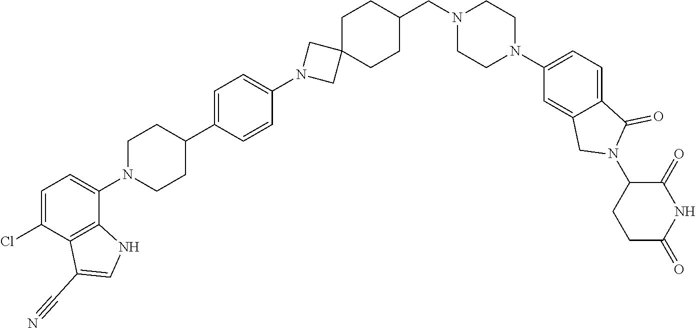 [SMILES: N#Cc1c[nH]c2c(N3CCC(c4ccc(N5CC6(CCC(CN7CCN(c8ccc9c(c8)CN(C8CCC(=O)NC8=O)C9=O)CC7)CC6)C5)cc4)CC3)ccc(Cl)c12]

The title compound was prepared using methodology described in example 128 using 4-chloro-7-(4-{4-[7-(dibutoxymethyl)-2-azaspiro[3.5]nonan-2-yl]phenyl}piperidin-1-yl)-1H-indole-3-carbonitrile and intermediate 1i to give the title compound in the form of a formate salt (9.5 mg, 9%) as a white solid. ¹H NMR δ 1.09 (2H, d), 1.54 (2H, t), 1.83 (5H, d), 1.89-2.11 (5H, m), 2.3-2.47 (1H, m), 2.60 (2H, d), 2.79 (2H, t), 2.85-2.99 (1H, m), 3.02 (2H, s), 3.13 (2H, d), 3.40 (4H, t), 3.56 (6H, d), 3.98 (2H, d), 4.24 (1H, d), 4.37 (1H, d), 5.07 (1H, dd), 6.51 (2H, s), 6.88 (1H, d), 7.1-7.21 (5H, m), 7.60 (1H, d), 8.34 (1H, d), 10.97 (1H, s), 12.33 (1H, s); m/z: ES⁺ [M+H]⁺=799.3.

### Example 130

### Intermediate 130a

### Benzyl (3S)-3-{4-[7-(dibutoxymethyl)-2-azaspiro[3.5]nonan-2-yl]phenyl}piperidine-1-carboxylate

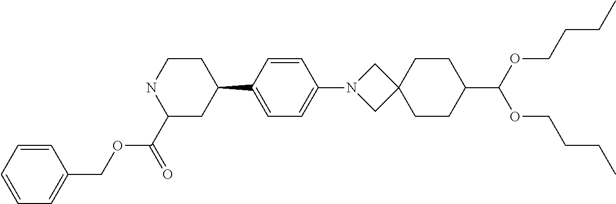 [SMILES: CCCCOC(OCCCC)C1CCC2(CC1)CN(c1ccc([C@@H]3CCNC(C(=O)OCc4ccccc4)C3)cc1)C2]

The title compound was prepared using methodology described in intermediate 11a using intermediate 128g and intermediate 9d to give the title compound (2.5 g, 90%) as a yellow gum. ¹H NMR δ 0.88 (5H, t), 1.03 (1H, q), 1.17 (2H, t), 1.32 (3H, qd), 1.36-1.46 (1H, m), 1.43-1.65 (5H, m), 1.68 (3H, dd), 1.85 (3H, t), 1.99 (2H, s), 2.42-2.84 (6H, m), 3.28-3.41 (4H, m), 3.46 (2H, s), 3.86-4.11 (2H, m), 5.09 (2H, d), 6.34 (2H, d), 7.02 (2H, d), 7.36 (5H, d); m/z: ES⁺ [M+H]⁺=577.4.

### Intermediate 130b

### 7-(Dibutoxymethyl)-2-{4-[(3S)-piperidin-3-yl]phenyl}-2-azaspiro[3.5]nonane

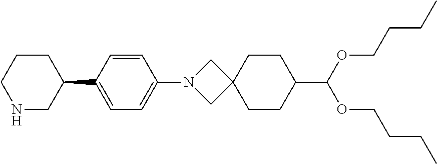 [SMILES: CCCCOC(OCCCC)C1CCC2(CC1)CN(c1ccc([C@@H]3CCCNC3)cc1)C2]

The title compound was prepared using methodology described in intermediate 60c using benzyl (3S)-3-{4-[7-(dibutoxymethyl)-2-azaspiro[3.5]nonan-2-yl]phenyl}piperidine-1-carboxylate to give the title compound (800 mg, 69%) as a yellow gum. ¹H NMR δ 0.89 (6H, t), 1.04 (2H, q), 1.13-1.55 (10H, m), 1.64 (3H, m), 1.73-1.94 (4H, m), 2.51 (3H, p), 2.66-2.99 (3H, m), 3.13-3.26 (2H, m), 3.28-3.92 (8H, m), 3.95-4.28 (1H, m), 6.37 (2H, d), 7-7.1 (2H, m); m/z: ES⁺ [M+H]⁺=443.2.

### Intermediate 130c

### 4-Chloro-7-[(3S)-3-{4-[7-(dibutoxymethyl)-2-azaspiro[3.5]nonan-2-yl]phenyl}piperidin-1-yl]-1H-indole-3-carbonitrile

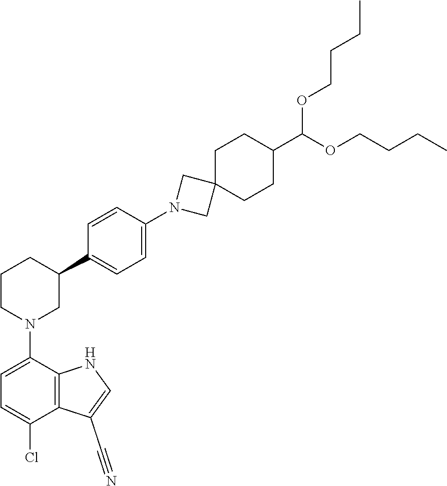 [SMILES: CCCCOC(OCCCC)C1CCC2(CC1)CN(c1ccc([C@@H]3CCCN(c4ccc(Cl)c5c(C#N)c[nH]c45)C3)cc1)C2]

The title compound was prepared using methodology described in intermediate 60d using 7-(dibutoxymethyl)-2-{4-[(3S)-piperidin-3-yl]phenyl}-2-azaspiro[3.5]nonane and intermediate 1b to give the title compound 150 mg, 43%) as a yellow gum. ¹H NMR δ 0.77 (1H, s), 0.88 (4H, t), 1.07 (1H, dd), 1.27-1.4 (2H, m), 1.4-1.53 (3H, m), 1.57 (2H, d), 1.65 (1H, d), 1.85-1.97 (7H, m), 2.18 (6H, t), 2.70 (8H, s), 3.36-3.6 (5H, m), 6.35 (1H, d), 6.83 (1H, dd), 6.96-7.26 (2H, m), 8.17 (3H, s), 8.40 (1H, d) 1H not observed; m/z: ES⁺ [M+H]⁺=617.3.

### Example 130

### 4-Chloro-7-[(3S)-3-{4-[7-({4-[2-(2,6-dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl]-1H-indole-3-carbonitrile

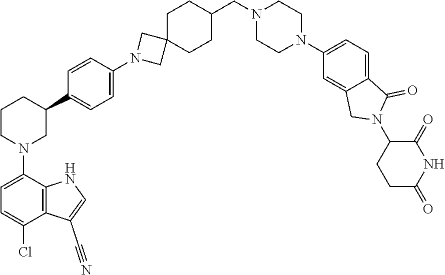 [SMILES: N#Cc1c[nH]c2c(N3CCC[C@@H](c4ccc(N5CC6(CCC(CN7CCN(c8ccc9c(c8)CN(C8CCC(=O)NC8=O)C9=O)CC7)CC6)C5)cc4)C3)ccc(Cl)c12]

The title compound was prepared using methodology described in example 128 using 4-chloro-7-[(3S)-3-{4-[7-(dibutoxymethyl)-2-azaspiro[3.5]nonan-2-yl]phenyl}piperidin-1-yl]-1H-indole-3-carbonitrile and intermediate 1i to give the title compound in the form of a formate salt (18 mg, 19%) as a white solid. ¹H NMR δ 1.04 (2H, s), 1.39 (1H, dd), 1.50 (4H, dt), 1.77 (3H, s), 1.88 (6H, d), 1.93-2.02 (1H, m), 2.31-2.49 (2H, m), 2.53-2.61 (3H, m), 2.62-2.74 (3H, m), 2.86-2.96 (2H, m), 2.99 (2H, d), 3.13 (1H, s), 3.37 (1H, s), 3.46 (4H, d), 3.58 (1H, s), 3.87-4.07 (1H, m), 4.23 (1H, d), 4.35 (1H, d), 5.07 (1H, dd), 5.76 (1H, s), 6.36 (2H, d), 6.83 (1H, d), 7.12 (1H, d), 7.14 (2H, d), 7.57 (1H, s), 8.32 (1H, d), 10.97 (1H, s), 12.31 (1H, s); m/z: ES⁺ [M+H]⁺=799.3.

### Example 131

### 4-Chloro-7-[(3S)-3-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl]-1H-indole-3-carbonitrile

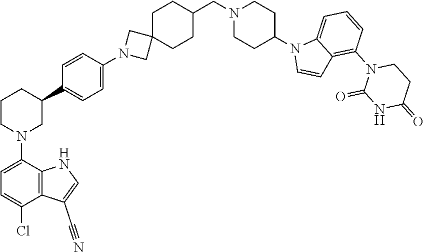 [SMILES: N#Cc1c[nH]c2c(N3CCC[C@@H](c4ccc(N5CC6(CCC(CN7CCC(n8ccc9c(N%10CCC(=O)NC%10=O)cccc98)CC7)CC6)C5)cc4)C3)ccc(Cl)c12]

The title compound was prepared using methodology described in example 63 using intermediate 130c and intermediate 5f to give the title compound (19 mg, 20%) as a white solid. ¹H NMR δ 1.09 (2H, s), 1.12-1.25 (1H, m), 1.54 (4H, d), 1.85 (4H, s), 1.9-1.95 (4H, m), 2.14 (2H, s), 2.51 (2H, d), 2.54-2.76 (3H, m), 2.77 (2H, t), 2.97 (3H, d), 3.30 (1H, d), 3.37 (1H, s), 3.47 (4H, d), 3.67 (1H, s), 3.78 (2H, t), 4.75 (1H, s), 6.37 (2H, d), 6.48 (1H, s), 6.91 (2H, dd), 7.08-7.21 (4H, m), 7.51 (2H, d), 8.32 (1H, d), 10.36 (1H, s), 12.32 (1H, d); m/z: ES⁺ [M+H]⁺=783.2.

### Example 132

### Intermediate 132a

### 4-Chloro-7-[(3R)-3-{4-[4-(dibutoxymethyl)piperidin-1-yl]phenyl}piperidin-1-yl]-1H-indazole-3-carbonitrile

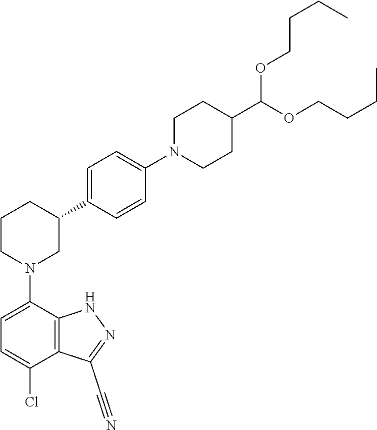 [SMILES: CCCCOC(OCCCC)C1CCN(c2ccc([C@H]3CCCN(c4ccc(Cl)c5c(C#N)n[nH]c45)C3)cc2)CC1]

The title compound was prepared using methodology described in example 60d using intermediate 21c and intermediate 9c give the title compound (180 mg, 31%) as a white solid. ¹H NMR δ 0.89 (6H, t), 1.22-1.42 (7H, m), 1.43-1.58 (5H, m), 1.59-1.79 (4H, m), 1.94 (3H, d), 2.56 (1H, d), 2.62-2.84 (2H, m), 3.00 (1H, t), 3.37-3.66 (7H, m), 4.18 (1H, d), 6.83-6.91 (2H, m), 6.94 (1H, d), 7.17 (2H, d), 7.28 (1H, d), 14.70 (1H, s); m/z: ES⁺ [M+H]⁺=578.0.

### Example 132

### 4-Chloro-7-[(3R)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile

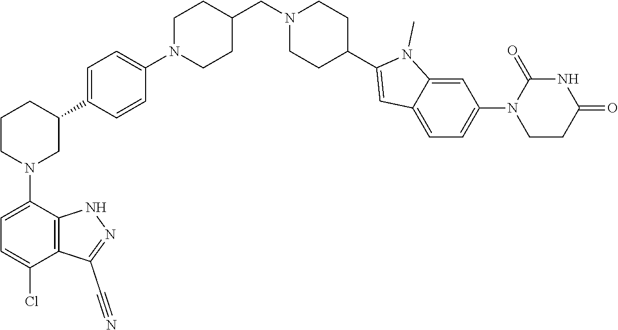 [SMILES: Cn1c(C2CCN(CC3CCN(c4ccc([C@H]5CCCN(c6ccc(Cl)c7c(C#N)n[nH]c67)C5)cc4)CC3)CC2)cc2ccc(N3CCC(=O)NC3=O)cc21]

4-Chloro-7-[(3R)-3-{4-[4-(dibutoxymethyl)piperidin-1-yl]phenyl}piperidin-1-yl]-1H-indazole-3-carbonitrile (180 mg, 0.31 mmol) and intermediate 13c (133 mg, 0.31 mmol) were stirred in formic acid (2 mL) at 40° C. for 1 h. The solvent was evaporated and the residue was diluted with NMP (2 mL). The mixture was stirred at RT for 16 h. Purification of the reaction mixture by preparative HPLC (Column A, Eluent A) gave the title compound in the form of a formate salt (40 mg, 16%) as a pale yellow solid. ¹H NMR δ 1.22 (2H, d), 1.63 (4H, d), 1.74-2 (7H, m), 2.06 (2H, d), 2.22 (2H, d), 2.56-2.84 (7H, m), 2.96 (3H, s), 3.53-3.88 (9H, m), 6.24 (1H, s), 6.77 (1H, s), 6.83-6.98 (3H, m), 7.17 (3H, d), 7.32-7.48 (2H, m), 10.29 (1H, s) 1H not observed; m/z: ES⁺ [M+H]⁺=758.6.

### Example 133

### Intermediate 133a

### 7-Bromo-4-fluoro-3-iodo-1H-indazole

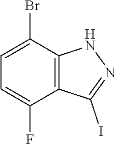 [SMILES: Fc1ccc(Br)c2[nH]nc(I)c12]

Iodine solution (23.6 g, 93.01 mmol) was added to 7-bromo-4-fluoro-1H-indazole (10.0 g, 46.51 mmol) and KOH (10.4 g, 186.02 mmol) in DMF (180 mL) at RT for 2 h. The reaction mixture was quenched with saturated Na₂S₂O₃ solution (200 mL), extracted with EtOAc (400 mL), dried over Na₂SO₄, filtered and evaporated to afford crude product. The crude product was purified by flash silica chromatography, elution gradient 0 to 30% EtOAc in Et₂O to give the title compound (8.0 g, 48.1%) as a white solid. ¹H NMR δ 2.89 (2H, s), 7.96 (1H, s); m/z: ES⁺ [M+H]⁺=340.7.

### Intermediate 133b

### 7-Bromo-4-fluoro-1H-indazole-3-carbonitrile

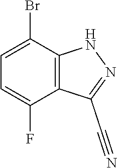 [SMILES: N#Cc1n[nH]c2c(Br)ccc(F)c12]

Tetrakis(triphenylphosphine)palladium(0) (0.814 g, 0.70 mmol), 7-bromo-4-fluoro-3-iodo-1H-indazole (4.8 g, 14.08 mmol) and zinc(II) cyanide (1.98 g, 16.90 mmol) were added to DMF (50 mL) and sealed into a microwave tube. The reaction was heated to 100° C. for 16 h in the microwave reactor and then cooled to RT. The reaction mixture was diluted with DCM (200 mL), washed with NaHCO₃ solution (100 mL), NaCl solution (50 mL), dried over Na₂SO₄, filtered and evaporated to afford crude product. The crude product was purified by flash silica chromatography, elution gradient 0 to 20% EtOAc in Et₂O to give the title compound (800 mg, 23%) as a white solid. ¹H NMR δ 7.18 (1H, m), 7.46-7.63 (1H, m), 15.15 (1H, s); m z ES⁻[M−H]⁻=237.9.

### Intermediate 133c

### 7-[(3S)-3-{4-[4-(Dibutoxymethyl)piperidin-1-yl]phenyl}piperidin-1-yl]-4-fluoro-1H-indazole-3-carbonitrile

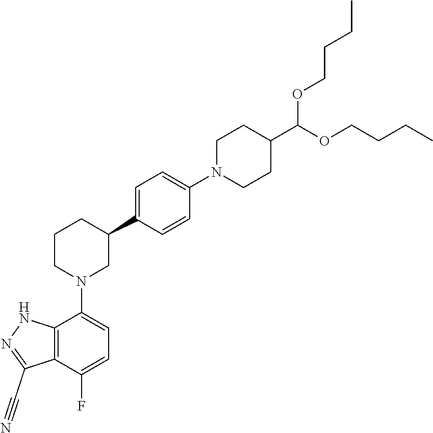 [SMILES: CCCCOC(OCCCC)C1CCN(c2ccc([C@@H]3CCCN(c4ccc(F)c5c(C#N)n[nH]c45)C3)cc2)CC1]

The title compound was prepared using methodology described in intermediate 60d using 7-bromo-4-fluoro-1H-indazole-3-carbonitrile and intermediate 11b to give the title compound (472 mg, 50%) as a yellow solid. ¹H NMR δ 0.88 (6H, t), 1.2-1.41 (7H, m), 1.42-1.56 (5H, m), 1.71 (3H, d), 1.79-2 (3H, m), 2.57-2.73 (2H, m), 3.00 (1H, t), 3.27-3.46 (5H, m), 3.51-3.7 (4H, m), 4.17 (1H, d), 6.85 (2H, d), 6.96-7.34 (4H, m), 14.69 (1H, s); m/z: ES⁺ [M+H]⁺=562.1.

### Example 133

### 7-[(3S)-3-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indazole-3-carbonitrile

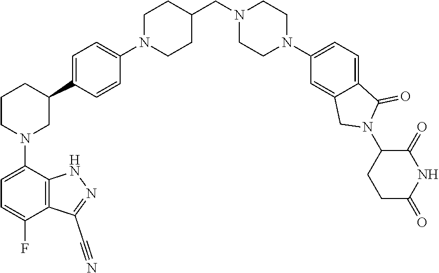 [SMILES: N#Cc1n[nH]c2c(N3CCC[C@@H](c4ccc(N5CCC(CN6CCN(c7ccc8c(c7)CN(C7CCC(=O)NC7=O)C8=O)CC6)CC5)cc4)C3)ccc(F)c12]

The title compound was prepared using methodology described in example 123 using 7-[(3S)-3-{4-[4-(dibutoxymethyl)piperidin-1-yl]phenyl}piperidin-1-yl]-4-fluoro-1H-indazole-3-carbonitrile and intermediate 1i to give the title compound in the form of a formate salt (98 mg, 37%) as a white solid. ¹H NMR δ 1.22 (2H, tt), 1.51-1.72 (2H, m), 1.75-2.01 (6H, m), 2.21 (2H, d), 2.36 (1H, tt), 2.51 (3H, s), 2.56-2.77 (5H, m), 2.83-3.03 (2H, m), 3.30 (4H, s), 3.45 (3H, d), 3.62 (2H, d), 4.16-4.38 (2H, m), 5.06 (1H, dd), 6.87 (3H, d), 6.97-7.1 (3H, m), 7.17 (2H, d), 7.53 (1H, d), 10.95 (1H, s), 14.67 (1H, s); m/z: ES⁺ [M+H]⁺=744.4.

### Example 134

### 7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indazole-3-carbonitrile

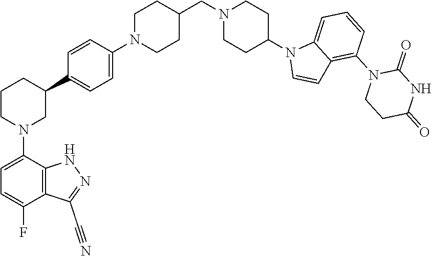 [SMILES: N#Cc1n[nH]c2c(N3CCC[C@@H](c4ccc(N5CCC(CN6CCC(n7ccc8c(N9CCC(=O)NC9=O)cccc87)CC6)CC5)cc4)C3)ccc(F)c12]

Intermediate 133c (235 mg, 0.42 mmol) was stirred in formic acid (1 mL) at 60° C. for 2 h. The mixture was evaporated to dryness and the residue was diluted with NMP (2 mL). Intermediate 5f (203 mg, 0.42 mmol) was added and the mixture was stirred at RT for 16 h. Purification of the reaction mixture by preparative HPLC (Column A, Eluent A) gave the title compound in the form of a formate salt (138 mg, 45.3%) as a white solid. ¹H NMR δ 1.21 (2H, q), 1.57 (2H, dd), 1.81 (2H, d), 1.97 (7H, h), 2.09-2.28 (4H, m), 2.55-2.85 (6H, m), 2.99 (3H, d), 3.45 (2H, t), 3.62 (2H, d), 3.78 (2H, t), 4.38 (1H, dq), 6.42 (1H, d), 6.87 (3H, d), 6.93-7.08 (2H, m), 7.16 (3H, d), 7.52 (2H, dd), 10.35 (1H, s), 14.63 (1H, s); m/z: ES⁺ [M+H]⁺=728.4.

### Example 135

### 7-(4-{4-[7-({4-[4-(2,4-Dioxo-1,3-diazinan-1-yl)-1H-indol-1-yl]piperidin-1-yl}methyl)-2-azaspiro[3.5]nonan-2-yl]phenyl}piperidin-1-yl)-4-fluoro-1H-indole-3-carbonitrile

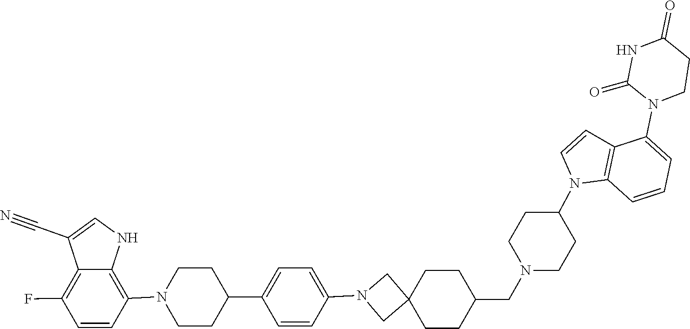 [SMILES: N#Cc1c[nH]c2c(N3CCC(c4ccc(N5CC6(CCC(CN7CCC(n8ccc9c(N%10CCC(=O)NC%10=O)cccc98)CC7)CC6)C5)cc4)CC3)ccc(F)c12]

The title compound was prepared using methodology described in example 134 using intermediate 128j and intermediate 5f to give the title compound in the form of a formate salt (7 mg, 5%) as a white solid. ¹H NMR δ 0.97 (2H, d), 1.44-1.55 (3H, m), 1.73 (4H, s), 1.75-1.86 (6 m), 1.90 (4H, d), 1.95 (4H, s), 2.16 (2H, dd), 2.7-2.8 (3H, m), 2.98 (2H, d), 3.39 (2H, d), 3.78 (3H, t), 4.37 (1H, s), 6.37-6.44 (3H, m), 6.81 (1H, d), 6.96 (2H, d), 7.06-7.17 (3H, m), 7.50 (1H, s), 7.55 (1H, d), 8.26 (1H, s), 8.46 (1H, s), 10.34 (1H, s) 1H not observed; m/z: ES⁺ [M+H]⁺=767.7.

### Example 136

### Intermediate 136a

### 5-Bromo-4-fluoro-2-iodoaniline

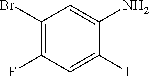

1-Bromo-2-fluoro-4-iodo-5-nitrobenzene (10.0 g, 28.91 mmol) was added to ammonium chloride (7.7 g, 144.55 mmol) and iron (8.0 g, 144.55 mmol) in MeOH (100 mL) and water (25.00 mL). The mixture was stirred at 60° C. for 1 h. The reaction mixture was filtered and the solvent was evaporated to afford crude product. The crude product was purified by flash silica chromatography, elution gradient 0 to 30% EtOAc in Et₂O to give the title compound (5.2 g, 56.9%) as a white solid. ¹H NMR δ 5.30 (2H, s), 7.00 (1H, d), 7.57 (1H, d); m/z: ES⁺ [M+H]⁺=315.7.

### Intermediate 136b

### tert-Butyl 4-[(2-amino-4-bromo-5-fluorophenyl)ethynyl]piperidine-1-carboxylate

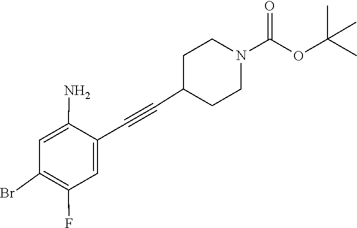 [SMILES: CC(C)(C)OC(=O)N1CCC(C#Cc2cc(F)c(Br)cc2N)CC1]

The title compound was prepared using methodology described in intermediate 103a using 5-bromo-4-fluoro-2-iodoaniline and tert-butyl 4-ethynylpiperidine-1-carboxylate to give the title compound (5.0 g, 76%) as a yellow solid. ¹H NMR δ 1.41 (9H, s), 1.49-1.63 (2H, m), 1.75-1.9 (2H, m), 2.87 (1H, dd), 3.10 (2H, t), 3.6-3.74 (2H, m), 5.36 (2H, s), 6.96 (1H, d), 7.11 (1H, d); m/z: ES⁺ [M+H]⁺=396.9.

### Intermediate 136c

### 4-(6-Bromo-5-fluoro-1H-indol-2-yl)piperidine-1-carboxylic acid

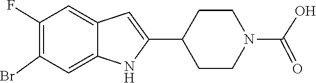 [SMILES: O=C(O)N1CCC(c2cc3cc(F)c(Br)cc3[nH]2)CC1]

Bis(acetonitrile)palladium(II) chloride (0.653 g, 2.52 mmol) was added to tert-butyl 4-[(2-amino-4-bromo-5-fluorophenyl)ethynyl]piperidine-1-carboxylate (5.0 g, 12.59 mmol) in DMF (80 mL) under nitrogen. The mixture was stirred at 120° C. for 3 h, cooled to RT and the solvent was evaporated to afford crude product. The crude product was purified by flash C18-flash chromatography, elution gradient 0 to 50% MeOH in water (0.1% formic acid) to give the title compound (700 mg, 16.3%) as a brown foam. ¹H NMR δ 1.79 (2H, q), 2.11 (2H, d), 2.86-3.05 (3H, m), 3.28 (2H, d), 6.14-6.29 (1H, m), 7.40 (1H, d), 7.55 (1H, dd), 8.40 (1H, d), 11.39 (1H, s); m/z: ES⁺ [M+H]⁺=341.0.

### Intermediate 136d

### 6-Bromo-5-fluoro-2-(piperidin-4-yl)-1H-indole

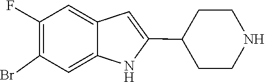 [SMILES: Fc1cc2cc(C3CCNCC3)[nH]c2cc1Br]

HCl (1.25M in MeOH, 3 mL, 12.00 mmol) was added to 4-(6-bromo-5-fluoro-1H-indol-2-yl)piperidine-1-carboxylic acid (200 mg, 0.59 mmol) and was stirred at RT for 1 h. The solvent was evaporated to afford crude product which was used without further purification. m/z: ES⁺ [M+H]⁺=297.0.

### Intermediate 136e

### tert-Butyl 4-(6-bromo-5-fluoro-1H-indol-2-yl)piperidine-1-carboxylate

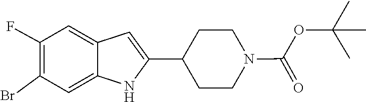 [SMILES: CC(C)(C)OC(=O)N1CCC(c2cc3cc(F)c(Br)cc3[nH]2)CC1]

DMAP (103 mg, 0.84 mmol) was added to 6-bromo-5-fluoro-2-(piperidin-4-yl)-1H-indole (500 mg, 1.68 mmol), di-tert-butyl dicarbonate (0.586 mL, 2.52 mmol) and DIPEA (0.882 mL, 5.05 mmol) in MeOH (5 mL) at RT for 16 h. The solvent was evaporated to afford to crude product. The crude product was purified by flash silica chromatography, elution gradient 0 to 50% EtOAc in Et₂O to give the title compound (300 mg, 44.9%) as a brown solid. ¹H NMR δ 1.42 (9H, s), 1.47-1.64 (2H, m), 1.9-2.03 (2H, m), 2.78-2.96 (3H, m), 4.04 (2H, d), 6.20 (1H, d), 7.38 (1H, d), 7.51 (1H, dd), 11.20 (1H, s).

### Intermediate 136f

### tert-Butyl 4-(6-bromo-5-fluoro-1-methyl-1H-indol-2-yl)piperidine-1-carboxylate

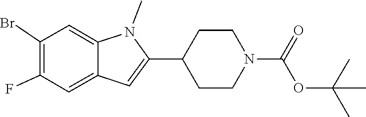 [SMILES: Cn1c(C2CCN(C(=O)OC(C)(C)C)CC2)cc2cc(F)c(Br)cc21]

The title compound was prepared using methodology described in intermediate 103c using tert-butyl 4-(6-bromo-5-fluoro-1H-indol-2-yl)piperidine-1-carboxylate to give the title compound (1.4 g, 67%) as a brown solid. ¹H NMR δ 1.43 (9H, s), 1.93 (2H, d), 2.74 (1H, t), 2.86-3.05 (2H, m), 3.72 (3H, d), 4.07 (2H, d), 5.76 (1H, s), 6.26 (1H, s), 7.31 (1H, d), 7.50 (1H, d), 10.43 (1H, s); m/z: ES⁺ [M+H]⁺=412.0.

### Intermediate 136g

### tert-Butyl 4-[6-(2,4-dioxo-1,3-diazinan-1-yl)-5-fluoro-1-methyl-1H-indol-2-yl]piperidine-1-carboxylate

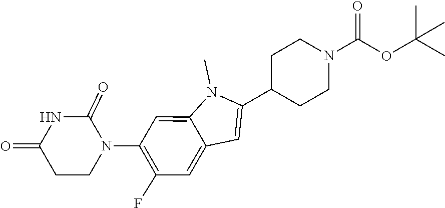 [SMILES: Cn1c(C2CCN(C(=O)OC(C)(C)C)CC2)cc2cc(F)c(N3CCC(=O)NC3=O)cc21]

The title compound was prepared using methodology described in intermediate 102b using tert-butyl 4-(6-bromo-5-fluoro-1-methyl-1H-indol-2-yl)piperidine-1-carboxylate and 1,3-diazinane-2,4-dione to give the title compound (750 mg, 35%) as a brown solid. ¹H NMR (CDCl₃) δ 6.16 (9H, s), 6.59-6.81 (2H, m), 7.24 (5H, p), 7.55-7.8 (3H, m), 8.45 (4H, d), 8.79 (2H, dd), 10.99 (1H, s), 12.04 (1H, d), 12.19-12.31 (1H, m), 15.16 (1H, s); m/z: ES⁺ [M-tert-Bu+H]⁺=389.0.

### Example 136

### 4-Chloro-7-[(3R)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indole-3-carbonitrile

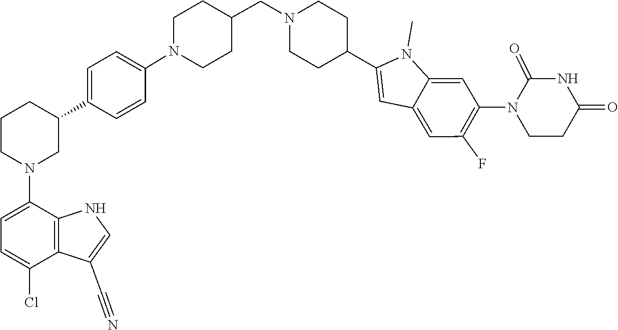 [SMILES: Cn1c(C2CCN(CC3CCN(c4ccc([C@H]5CCCN(c6ccc(Cl)c7c(C#N)c[nH]c67)C5)cc4)CC3)CC2)cc2cc(F)c(N3CCC(=O)NC3=O)cc21]

The title compound was prepared using methodology described in example 123 using tert-butyl 4-[6-(2,4-dioxo-1,3-diazinan-1-yl)-5-fluoro-1-methyl-1H-indol-2-yl]piperidine-1-carboxylate and intermediate 21d to give the title compound in the form of a formate salt (35 mg, 29%) as a white solid. ¹H NMR δ 1.14-1.29 (2H, m), 1.56 (1H, t), 1.62-1.71 (3H, m), 1.75-1.84 (3H, m), 1.91 (4H, s), 2.06 (2H, t), 2.20 (2H, d), 2.63 (4H, dt), 2.75 (3H, q), 2.95 (3H, d), 3.41 (2H, d), 3.62 (2H, d), 3.71 (5H, d), 6.25 (1H, s), 6.79 (1H, d), 6.87 (2H, d), 7.09 (1H, d), 7.16 (2H, d), 7.30 (1H, d), 7.49 (1H, d), 8.26 (1H, s), 10.42 (1H, s), 12.38 (1H, s); m/z: ES⁺ [M+H]⁺=775.4.

### Example 137

### Intermediate 137a

### 4-Chloro-7-{(3S)-3-[4-(4-formylpiperidin-1-yl)phenyl]piperidin-1-yl}-1H-indole-3-carbonitrile

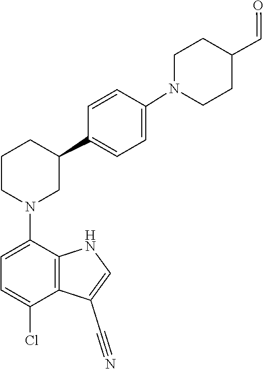 [SMILES: N#Cc1c[nH]c2c(N3CCC[C@@H](c4ccc(N5CCC(C=O)CC5)cc4)C3)ccc(Cl)c12]

Intermediate 11c (200 mg, 0.35 mmol) was stirred in formic acid (0.5 mL) at 60° C. for 2 h. The reaction was cooled to RT and evaporated to dryness to give the title compound as a crude solid which was used without further purification. ¹H NMR δ 1.08-1.31 (1H, m), 1.46-1.68 (2H, m), 1.77-2.03 (4H, m), 2.42-2.55 (4H, m), 2.6-2.85 (3H, m), 3.00 (1H, t), 3.34 (2H, t), 3.46-3.59 (2H, m), 6.72-6.96 (2H, m), 7.15 (2H, dd), 8.31 (1H, d), 9.63 (1H, d), 12.75 (2H, s); m/z: ES⁺ [M+H]⁺=447.0.

### Example 137

### 4-Chloro-7-[(3S)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indole-3-carbonitrile

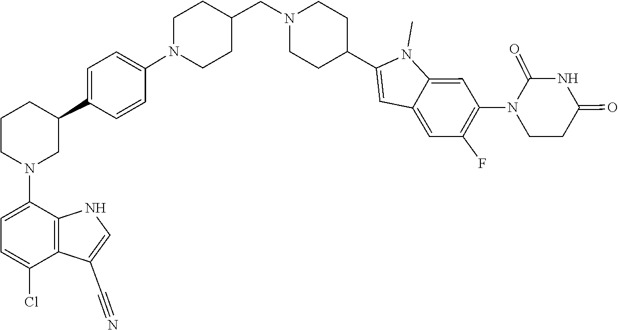 [SMILES: Cn1c(C2CCN(CC3CCN(c4ccc([C@@H]5CCCN(c6ccc(Cl)c7c(C#N)c[nH]c67)C5)cc4)CC3)CC2)cc2cc(F)c(N3CCC(=O)NC3=O)cc21]

The title compound was prepared using methodology described in example 123 using 4-chloro-7-{(3S)-3-[4-(4-formylpiperidin-1-yl)phenyl]piperidin-1-yl}-1H-indole-3-carbonitrile and intermediate 136g to give the title compound in the form of a formate salt (75 mg, 28%) as a white solid. ¹H NMR δ 1.21 (2H, t), 1.51-1.71 (4H, m), 1.79 (2H, d), 1.91 (5H, q), 2.05 (2H, t), 2.19 (2H, d), 2.52 (1H, s), 2.55-2.7 (4H, m), 2.74 (3H, t), 2.96 (3H, t), 3.26-3.4 (3H, m), 3.62 (2H, d), 3.70 (1H, s), 3.73 (2H, d), 6.25 (1H, s), 6.85 (3H, dd), 7.15 (3H, dd), 7.30 (1H, d), 7.49 (1H, d), 8.32 (1H, s), 10.43 (1H, s), 12.29 (1H, s); m/z: ES⁺ [M+H]⁺=775.4.

### Example 138

### 4-Chloro-7-[(3R)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indazole-3-carbonitrile

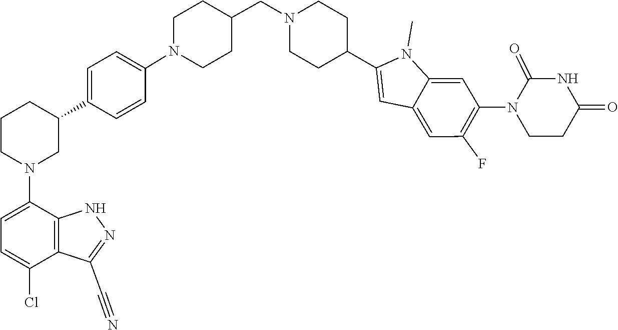 [SMILES: Cn1c(C2CCN(CC3CCN(c4ccc([C@H]5CCCN(c6ccc(Cl)c7c(C#N)n[nH]c67)C5)cc4)CC3)CC2)cc2cc(F)c(N3CCC(=O)NC3=O)cc21]

The title compound was prepared using methodology described in example 123 using intermediate 132a and intermediate 136g to give the title compound in the form of a formate salt (153 mg, 44%) as a white solid. ¹H NMR δ 1.22 (2H, q), 1.64 (4H, dt), 1.80 (2H, d), 1.91 (5H, t), 2.11 (2H, t), 2.24 (2H, d), 2.57-2.82 (7H, m), 2.98 (3H, d), 3.53 (2H, t), 3.63 (2H, d), 3.72 (5H, t), 6.26 (1H, s), 6.89 (3H, dd), 7.18 (2H, d), 7.25 (1H, d), 7.31 (1H, d), 7.49 (1H, d), 10.42 (1H, s), 14.44 (1H, s); m/z: ES⁺ [M+H]⁺=776.5.

### Example 139

### 4-Chloro-7-[(3S)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indazole-3-carbonitrile

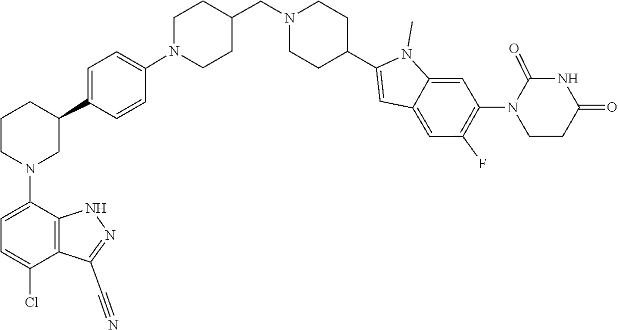 [SMILES: Cn1c(C2CCN(CC3CCN(c4ccc([C@@H]5CCCN(c6ccc(Cl)c7c(C#N)n[nH]c67)C5)cc4)CC3)CC2)cc2cc(F)c(N3CCC(=O)NC3=O)cc21]

The title compound was prepared using methodology described in example 123 using intermediate 9g and intermediate 136g to give the title compound in the form of a formate salt (94 mg, 24%) as a white solid. ¹H NMR δ 1.23 (2H, t), 1.56-1.72 (4H, m), 1.80 (2H, d), 1.91 (5H, t), 2.10 (2H, t), 2.24 (2H, d), 2.58-2.83 (7H, m), 2.98 (3H, d), 3.49-3.57 (2H, m), 3.63 (2H, d), 3.71 (5H, d), 6.26 (1H, s), 6.88 (3H, dd), 7.18 (2H, d), 7.24 (1H, d), 7.31 (1H, d), 7.49 (1H, d), 10.43 (1H, s), 14.45 (1H, s); m/z: ES⁺ [M+H]⁺=776.4.

### Example 140

### Intermediate 140a

### 7-(4-{4-[4-(Dibutoxymethyl)piperidin-1-yl]phenyl}piperidin-1-yl)-4-fluoro-1H-indazole-3-carbonitrile

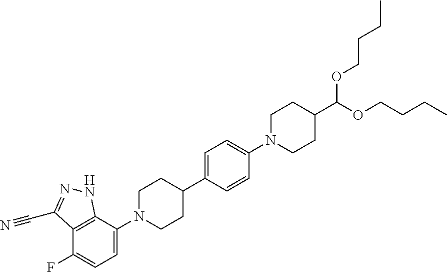 [SMILES: CCCCOC(OCCCC)C1CCN(c2ccc(C3CCN(c4ccc(F)c5c(C#N)n[nH]c45)CC3)cc2)CC1]

The title compound was prepared using methodology described in intermediate 60d using intermediate 133b and intermediate 1e to give the title compound (210 mg, 33%) as a yellow solid. ¹H NMR (CDCl₃) δ 0.96 (8H, t), 1.28 (1H, s), 1.39-1.47 (4H, m), 1.56-1.62 (4H, m), 1.78 (1H, dtd), 1.91 (2H, d), 2.01 (3H, s), 2.67 (3H, dt), 2.91 (2H, td), 3.42-3.54 (5H, m), 3.61-3.74 (4H, m), 4.23 (1H, d), 6.87-6.99 (4H, m), 7.14-7.21 (2H, m); m/z: ES⁺ [M+H]⁺=562.4.

### Example 140

### 7-(4-{4-[4-({4-[6-(2,4-Dioxo-1,3-diazinan-1-yl)-1-methyl-1H-indol-2-yl]piperidin-1-yl}methyl)piperidin-1-yl]phenyl}piperidin-1-yl)-4-fluoro-1H-indazole-3-carbonitrile

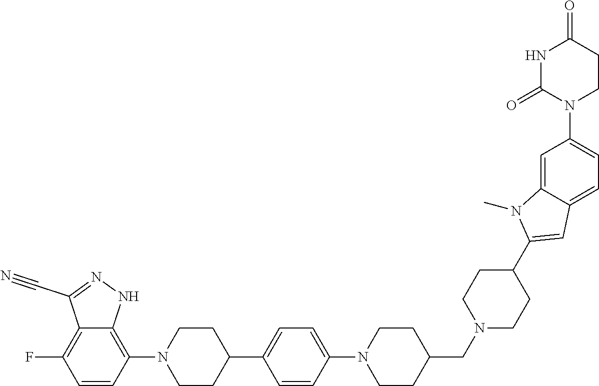 [SMILES: Cn1c(C2CCN(CC3CCN(c4ccc(C5CCN(c6ccc(F)c7c(C#N)n[nH]c67)CC5)cc4)CC3)CC2)cc2ccc(N3CCC(=O)NC3=O)cc21]

7-(4-{4-[4-(Dibutoxymethyl)piperidin-1-yl]phenyl}piperidin-1-yl)-4-fluoro-1H-indazole-3-carbonitrile (100 mg, 0.18 mmol) was stirred in formic acid (0.5 mL) at 60° C. for 2 h. The reaction was cooled to RT and evaporated to dryness. Intermediate 13c (200 mg, 0.47 mmol) was stirred in formic acid (2 mL) at 40° C. for 1 h. The solvent was evaporated to dryness. The two residues were dissolved in NMP (1.5 mL) and sodium acetate (19.01 mg, 0.23 mmol) was added. The resulting solution was stirred at RT for 16 h. Purification of the reaction mixture by preparative HPLC (Column A, Eluent A) gave the title compound in the form of a formate salt (41 mg, 23%) as a white solid. ¹H NMR δ 1.24 (2H, s), 1.67 (3H, s), 1.84 (4H, t), 1.94 (4H, s), 2.11 (2H, s), 2.25 (2H, s), 2.57-2.68 (4H, m), 2.69-2.85 (5H, m), 2.95-3.03 (2H, m), 3.56 (2H, s), 3.66 (5H, d), 3.79 (2H, s), 6.24 (1H, s), 6.91 (4H, d), 7.03 (1H, s), 7.14 (2H, d), 7.36 (1H, s), 7.43 (1H, d), 10.26-10.31 (1H, m); m/z: ES⁺ [M+H]⁺=742.4.

### Example 141

### 7-(4-{4-[4-({4-[4-(2,4-Dioxo-1,3-diazinan-1-yl)-2-methyl-1H-indol-1-yl]piperidin-1-yl}methyl)piperidin-1-yl]phenyl}piperidin-1-yl)-4-fluoro-1H-indole-3-carbonitrile

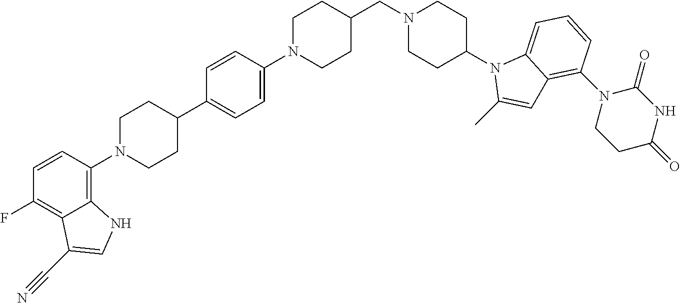 [SMILES: Cc1cc2c(N3CCC(=O)NC3=O)cccc2n1C1CCN(CC2CCN(c3ccc(C4CCN(c5ccc(F)c6c(C#N)c[nH]c56)CC4)cc3)CC2)CC1]

A suspension of intermediate 2d (82 mg, 0.19 mmol) and intermediate 114e (100 mg, 0.20 mmol) in NMP (1 mL) was stirred at RT for 5 mins followed by the addition of sodium triacetoxyborohydride (48 mg, 0.23 mmol) and was stirred at RT for 1 h. The mixture was added dropwise to a stirred solution of sat. NaHCO₃:water (1:2, 5 mL). The resulting solid was filtered, washing with water and then dried under vacuum at 45° C. to afford crude product. Purification by preparative HPLC (Column A, Eluent A) gave the title compound in the form of a formate salt (83 mg, 58.6%) as a white solid. ¹H NMR δ 1.19-1.32 (3H, m), 1.62-1.73 (1H, m), 1.73-1.81 (2H, m), 1.86 (4H, s), 1.9-2.04 (2H, m), 2.14 (2H, t), 2.26 (2H, d), 2.38-2.47 (4H, m), 2.53-2.69 (3H, m), 2.7-2.83 (4H, m), 3.03 (2H, d), 3.38 (2H, d), 3.67 (2H, d), 3.74 (2H, t), 4.24 (1H, s), 6.16 (1H, s), 6.83 (1H, dd), 6.87-6.96 (4H, m), 7.03-7.09 (1H, m), 7.14 (2H, d), 7.48 (1H, d), 8.26 (1H, s), 10.29 (1H, s), 12.24 (1H, s); m/z: ES⁺ [M+H]⁺=741.5.

### Example 142

### 4-Chloro-7-(4-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl)-1H-indole-3-carbonitrile

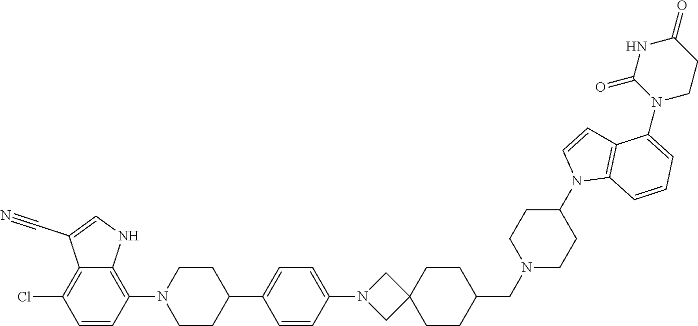 [SMILES: N#Cc1c[nH]c2c(N3CCC(c4ccc(N5CC6(CCC(CN7CCC(n8ccc9c(N%10CCC(=O)NC%10=O)cccc98)CC7)CC6)C5)cc4)CC3)ccc(Cl)c12]

Intermediate 129a (80 mg, 0.13 mmol) was stirred in formic acid (1 mL) at 60° C. for 2 h. The solvent was evaporated and diluted with NMP (3 mL). Intermediate 5f (75 mg, 0.16 mmol) and sodium acetate (10.63 mg, 0.13 mmol) was added and stirred at RT for 16 h. The reaction mixture was purified by flash C18-flash chromatography, elution gradient 0 to 50% MeCN in water (0.1% formic acid). Pure fractions were evaporated to dryness to give the title compound in the form of a formate salt (8.5 mg, 8.37%) as a white solid. ¹H NMR δ 1.11 (2H, d), 1.55 (2H, t), 1.85 (5H, s), 1.88 (OH, s), 1.97 (5H, d), 2.15 (2H, d), 2.34 (1H, d), 2.56 (1H, d), 2.64 (2H, d), 2.73-2.84 (4H, m), 2.99 (2H, d), 3.18 (2H, d), 3.43 (3H, d), 3.55 (4H, d), 3.79 (2H, t), 4.74 (1H, d), 6.50 (2H, dd), 6.88 (1H, d), 7.00 (1H, dd), 7.05-7.23 (4H, m), 7.40 (1H, d), 7.62 (1H, dd), 8.33 (1H, d), 10.36 (1H, s), 12.32 (1H, s); m/z: ES⁺ [M+H]⁺=783.3.

### Example 143

### Intermediate 143a

### Benzyl 4-{4-[4-(dibutoxymethyl)piperidin-1-yl]phenyl}piperidine-1-carboxylate

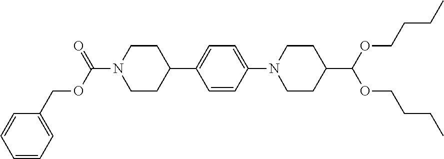 [SMILES: CCCCOC(OCCCC)C1CCN(c2ccc(C3CCN(C(=O)OCc4ccccc4)CC3)cc2)CC1]

The title compound was prepared using methodology described in intermediate 11a using 4-(dibutoxymethyl)piperidine and intermediate 1c to give the title compound (1.6 g, 37%) as a brown oil. ¹H NMR δ 0.89 (6H, t), 1.33 (6H, m), 1.46-1.52 (5H, m), 1.72 (4H, d), 2.57 (2H, dd), 2.88 (2H, s), 3.35 (3H, s), 3.39 (2H, dt), 3.55 (2H, dt), 3.62 (2H, d), 4.12 (2H, d), 4.18 (1H, d), 5.09 (2H, s), 6.84 (2H, d), 7.05 (2H, d), 7.33-7.43 (5H, m); m/z: ES⁺ [M+H]⁺=537.5.

### Intermediate 143b

### 4-(Dibutoxymethyl)-1-[4-(piperidin-4-yl)phenyl]piperidine

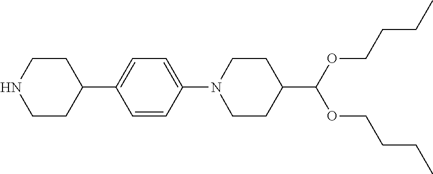 [SMILES: CCCCOC(OCCCC)C1CCN(c2ccc(C3CCNCC3)cc2)CC1]

The title compound was prepared using methodology described in intermediate 41c using benzyl 4-{4-[4-(dibutoxymethyl)piperidin-1-yl]phenyl}piperidine-1-carboxylate to give the title compound (950 mg, 84%) as a colourless gum. ¹H NMR δ 0.87 (6H, t), 0.99-1.55 (13H, m), 1.55-1.75 (5H, m), 2.39 (1H, d), 2.54 (3H, d), 2.97 (2H, d), 3.37 (2H, dt), 3.47-3.59 (3H, m), 3.61 (1H, s), 4.17 (1H, d), 6.77-6.88 (2H, m), 6.97-7.06 (2H, m); m/z: ES⁺ [M+H]⁺=403.3.

### Intermediate 143c

### 4-Chloro-7-(4-{4-[4-(dibutoxymethyl)piperidin-1-yl]phenyl}piperidin-1-yl)-1H-indazole-3-carbonitrile

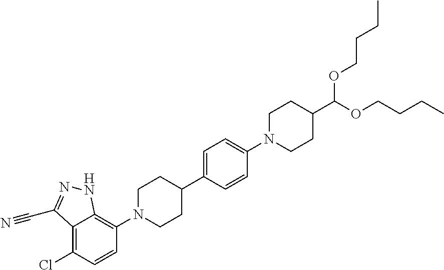 [SMILES: CCCCOC(OCCCC)C1CCN(c2ccc(C3CCN(c4ccc(Cl)c5c(C#N)n[nH]c45)CC3)cc2)CC1]

The title compound was prepared using methodology described in intermediate 57b using 4-(dibutoxymethyl)-1-[4-(piperidin-4-yl)phenyl]piperidine and intermediate 9c to give the title compound (220 mg, 39%) as a yellow solid. ¹H NMR δ 0.89 (6H, t), 1.21-1.44 (8H, m), 1.43-1.57 (4H, m), 1.69-1.78 (1H, m), 1.86-2.03 (4H, m), 2.53-2.63 (3H, m), 2.82 (2H, t), 3.33-3.45 (4H, m), 3.5-3.62 (2H, m), 3.65 (2H, d), 4.20 (1H, d), 6.89 (1H, s), 6.88-7 (2H, m), 7.13 (2H, d), 7.32 (1H, d), 14.67 (1H, s). no mass data

### Example 143

### 4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indazole-3-carbonitrile

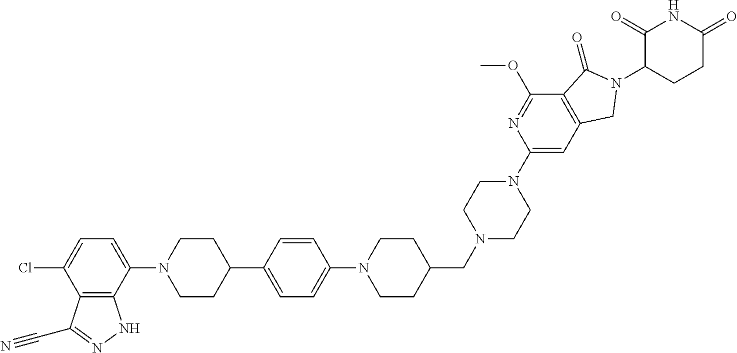 [SMILES: COc1nc(N2CCN(CC3CCN(c4ccc(C5CCN(c6ccc(Cl)c7c(C#N)n[nH]c67)CC5)cc4)CC3)CC2)cc2c1C(=O)N(C1CCC(=O)NC1=O)C2]

The title compound was prepared using methodology described in example 140 using 4-chloro-7-(4-{4-[4-(dibutoxymethyl)piperidin-1-yl]phenyl}piperidin-1-yl)-1H-indazole-3-carbonitrile and intermediate 30e to give the title compound (16 mg, 9%) as a white solid. ¹H NMR δ 1.23 (2H, q), 1.69 (1H, s), 1.77-2.01 (7H, m), 2.22 (2H, d), 2.31 (1H, dd), 2.42-2.48 (4H, m), 2.57-2.67 (4H, m), 2.76-2.94 (3H, m), 3.62 (8H, d), 3.89 (3H, s), 4.13 (1H, d), 4.27 (1H, d), 4.96 (1H, dd), 6.48 (1H, s), 6.91 (3H, dd), 7.13 (2H, d), 7.29 (1H, d), 10.92 (1H, s) one proton exchanged; m/z: ES⁺ [M+H]⁺=791.4.

### Example 144

### 7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indazole-3-carbonitrile

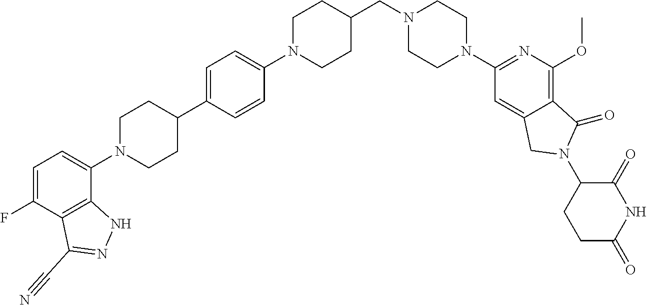 [SMILES: COc1nc(N2CCN(CC3CCN(c4ccc(C5CCN(c6ccc(F)c7c(C#N)n[nH]c67)CC5)cc4)CC3)CC2)cc2c1C(=O)N(C1CCC(=O)NC1=O)C2]

The title compound was prepared using methodology described in example 140 using intermediate 140a and intermediate 30e to give the title compound (18 mg, 16%) as a white solid. ¹H NMR δ 1.25 (2H, q), 1.69 (1H, s), 1.82-1.96 (7H, m), 2.22 (2H, d), 2.24-2.38 (1H, m), 2.45 (4H, t), 2.54-2.69 (5H, m), 2.78 (2H, t), 2.83-2.96 (1H, m), 3.58-3.68 (7H, m), 3.89 (3H, s), 4.13 (1H, d), 4.27 (1H, d), 4.96 (1H, dd), 6.49 (1H, s), 6.8-6.95 (3H, m), 7.01 (1H, t), 7.13 (2H, d), 10.92 (1H, s), 14.53 (1H, s); m/z: ES⁺ [M+H]⁺=775.4.

### Example 145: 7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indazole-3-carbonitrile

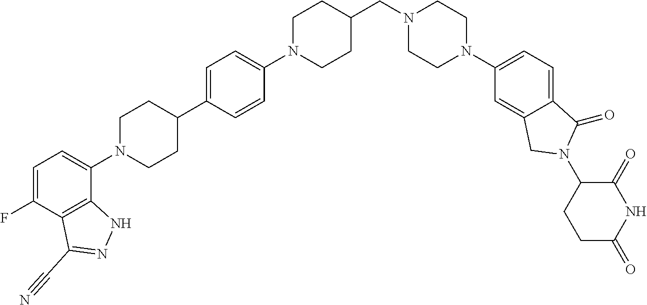 [SMILES: N#Cc1n[nH]c2c(N3CCC(c4ccc(N5CCC(CN6CCN(c7ccc8c(c7)CN(C7CCC(=O)NC7=O)C8=O)CC6)CC5)cc4)CC3)ccc(F)c12]

The title compound was prepared using methodology described in example 140 using intermediate 140a and intermediate 1i to give the title compound (20 mg, 18%) as a white solid. ¹H NMR δ 1.24 (3H, tq), 1.69 (1H, d), 1.78-2 (7H, m), 2.23 (2H, d), 2.29-2.45 (1H, m), 2.53 (1H, s), 2.62 (4H, td), 2.79 (2H, dd), 2.91 (1H, ddd), 3.30 (6H, d), 3.55 (2H, d), 3.65 (2H, d), 4.15-4.41 (2H, m), 5.06 (1H, dd), 6.90 (3H, td), 6.98-7.18 (5H, m), 7.53 (1H, d), 10.96 (1H, s), 14.51 (1H, s); m/z: ES⁻[M−H]⁻=742.2.

### Example 146

### 4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indazole-3-carbonitrile

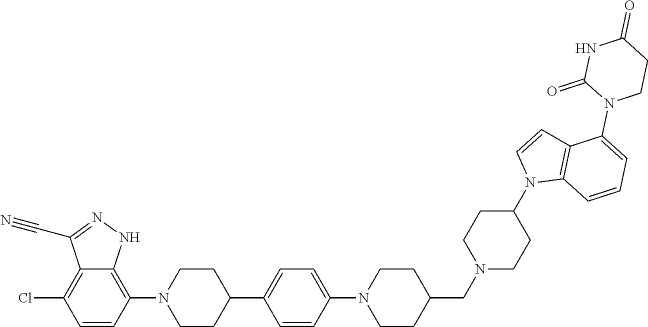 [SMILES: N#Cc1n[nH]c2c(N3CCC(c4ccc(N5CCC(CN6CCC(n7ccc8c(N9CCC(=O)NC9=O)cccc87)CC6)CC5)cc4)CC3)ccc(Cl)c12]

The title compound was prepared using methodology described in example 62 using intermediate 143c and intermediate 5f to give the title compound (15 mg, 19%) as a white solid. ¹H NMR δ 1.24 (2H, d), 1.68 (1H, s), 1.81-2.07 (10H, m), 2.23 (4H, dd), 2.6-2.69 (3H, m), 2.72-2.87 (4H, m), 3.02 (2H, d), 3.62 (4H, dd), 3.78 (2H, t), 4.37-4.41 (1H, m), 6.42 (1H, d), 6.88-7 (4H, m), 7.1-7.19 (3H, m), 7.29 (1H, d), 7.48-7.57 (2H, m), 10.34 (1H, s), 14.55 (1H, s); m/z: ES⁺ [M+H]⁺=744.4.

### Example 147

### 7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indazole-3-carbonitrile

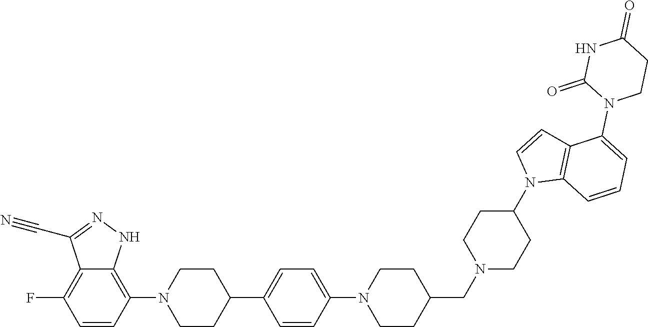 [SMILES: N#Cc1n[nH]c2c(N3CCC(c4ccc(N5CCC(CN6CCC(n7ccc8c(N9CCC(=O)NC9=O)cccc87)CC6)CC5)cc4)CC3)ccc(F)c12]

The title compound was prepared using methodology described in example 62 using intermediate 140a and intermediate 5f to give the title compound (15 mg, 19%) as a white solid. ¹H NMR δ 1.25 (2H, q), 1.69 (1H, s), 1.82-1.88 (4H, m), 1.91-2.09 (6H, m), 2.20 (2H, t), 2.28 (2H, d), 2.55-2.69 (3H, m), 2.74-2.85 (4H, m), 3.03 (2H, d), 3.53 (2H, d), 3.66 (2H, d), 3.79 (2H, t), 4.37-4.42 (1H, m), 6.43 (1H, d), 6.87-7.01 (4H, m), 7.06 (1H, t), 7.11-7.2 (3H, m), 7.49-7.58 (2H, m), 10.34 (1H, s), 14.55 (1H, s); m/z: ES⁺ [M+H]⁺=728.4.

### Example 148

### Intermediate 148a

### 7-Chloro-4-methyl-1H-indazole

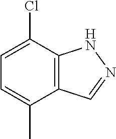

Pd(dppf)Cl₂ (0.632 g, 0.86 mmol) was added to K₂CO₃ (2.388 g, 17.28 mmol), 4-bromo-7-chloro-1H-indazole (2.0 g, 8.64 mmol) and trimethylboroxine (1.62 g, 12.96 mmol) in 1,4-dioxane:water (4:1, 30 mL) under nitrogen. The mixture was stirred at 120° C. for 16 h and then cooled to RT. The reaction mixture was diluted with EtOAc (50 mL) and washed with water (50 mL), NaCl solution, dried over MgSO₄, filtered and evaporated to afford crude product. The crude product was purified by flash silica chromatography, elution gradient 0 to 20% EtOAc in Et₂O to give the title compound (1.3 g, 90%) as a white solid. ¹H NMR δ 2.54 (3H, s), 6.89 (1H, d), 7.31 (1H, d), 8.23 (1H, d), 13.53 (1H, s); m/z: ES⁺ [M+H]⁺=167.1.

### Intermediate 148b

### 7-Chloro-3-iodo-4-methyl-1H-indazole

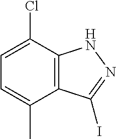

The title compound was prepared using methodology described in intermediate 113a using 7-chloro-4-methyl-1H-indazole to give the title compound (1.9 g, 87%) as a yellow solid. ¹H NMR δ 2.72 (3H, s), 6.83 (1H, dd), 7.31 (1H, d) 1H not observed; m/z: ES⁺ [M+H]⁺=292.9.

### Intermediate 148c

### 7-Chloro-4-methyl-1H-indazole-3-carbonitrile

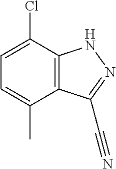

The title compound was prepared using methodology described in intermediate 113b using 7-chloro-3-iodo-4-methyl-1H-indazole to give the title compound (590 mg, 53%) as a white solid. ¹H NMR δ 2.67 (3H, d), 7.12 (1H, d), 7.51 (1H, d), 14.85 (1H, s); m/z: ES⁺ [M+H]⁺=192.0.

### Intermediate 148d

### 7-{4-[4-(Benzyloxy)phenyl]piperidin-1-yl}-4-methyl-1H-indazole-3-carbonitrile

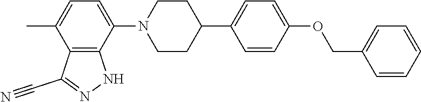 [SMILES: Cc1ccc(N2CCC(c3ccc(OCc4ccccc4)cc3)CC2)c2[nH]nc(C#N)c12]

The title compound was prepared using methodology described in intermediate 23a using 7-chloro-4-methyl-1H-indazole-3-carbonitrile and intermediate 27b to give the title compound (380 mg, 45%) as a white solid. ¹H NMR δ 1.80-2.00 (4H, m), 2.60 (3H, s), 2.59-2.66 (1H, m), 2.67-2.83 (2H, m), 3.44-3.54 (2H, m), 5.07 (2H, s), 6.82-6.91 (1H, m), 6.92-7.03 (3H, m), 7.16-7.25 (2H, m), 7.28-7.48 (5H, m), 14.26 (1H, s); m/z: ES⁺ [M+H]⁺=423.2.

### Intermediate 148e

### 7-{4-[4-(Benzyloxy)phenyl]piperidin-1-yl}-4-methyl-1-{[2-(trimethylsilyl)ethoxy]methyl}-1H-indazole-3-carbonitrile

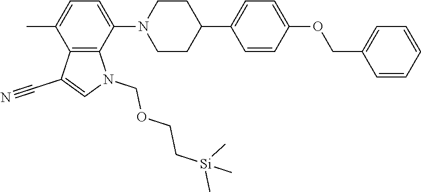 [SMILES: Cc1ccc(N2CCC(c3ccc(OCc4ccccc4)cc3)CC2)c2c1c(C#N)cn2COCC[Si](C)(C)C]

The title compound was prepared using methodology described in intermediate 27d using 7-chloro-4-methyl-1H-indazole-3-carbonitrile to give the title compound (400 mg, 87%) as a white solid. ¹H NMR δ −0.12 (9H, s), 0.83 (2H, dt), 1.84 (4H, s), 2.48-2.54 (1H, m), 2.63 (3H, s), 2.82 (2H, s), 3.24 (2H, d), 3.66 (2H, t), 5.06 (2H, d), 6.09 (2H, s), 6.88-6.99 (2H, m), 7.10 (1H, d), 7.16-7.26 (3H, m), 7.28-7.38 (3H, m), 7.39-7.48 (2H, m); m/z: ES⁺ [M+H]⁺=553.3.

### Intermediate 148f

### 7-[4-(4-Hydroxyphenyl)piperidin-1-yl]-4-methyl-1-{[2-(trimethylsilyl)ethoxy]methyl}-1H-indazole-3-carbonitrile

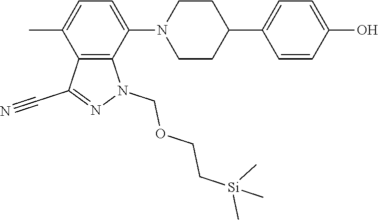 [SMILES: Cc1ccc(N2CCC(c3ccc(O)cc3)CC2)c2c1c(C#N)nn2COCC[Si](C)(C)C]

The title compound was prepared using methodology described in intermediate 27e using 7-{4-[4-(benzyloxy)phenyl]piperidin-1-yl}-4-methyl-1-{[2-(trimethylsilyl)ethoxy]methyl}-1H-indazole-3-carbonitrile to give the title compound (280 mg, 96%) as a yellow solid. ¹H NMR δ −0.08 (9H, s), 0.82 (2H, t), 1.79-1.89 (4H, m), 2.55-2.61 (1H, m), 2.65 (3H, s), 2.79-2.89 (2H, m), 3.25 (2H, d), 3.68 (2H, t), 6.10 (2H, s), 6.67-6.76 (2H, m), 7.06-7.15 (3H, m), 7.26 (1H, d), 9.28 (1H, s); m/z: ES⁺ [M+H]⁺=463.2.

### Intermediate 148g

### 7-(4-{4-[3-(1,3-Dioxolan-2-yl)propoxy]phenyl}piperidin-1-yl)-4-methyl-1-{[2-(trimethylsilyl)ethoxy]methyl}-1H-indazole-3-carbonitrile

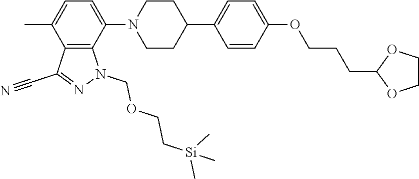 [SMILES: Cc1ccc(N2CCC(c3ccc(OCCCC4OCCO4)cc3)CC2)c2c1c(C#N)nn2COCC[Si](C)(C)C]

K₂CO₃ (233 mg, 1.69 mmol) was added to 7-[4-(4-hydroxyphenyl)piperidin-1-yl]-4-methyl-1-{[2-(trimethylsilyl)ethoxy]methyl}-1H-indazole-3-carbonitrile (260 mg, 0.56 mmol) and 2-(3-bromopropyl)-1,3-dioxolane (219 mg, 1.12 mmol) in MeCN (15 mL). The resulting mixture was stirred at 80° C. for 16 h and then cooled to RT. The solvent was evaporated to afford crude product. The crude product was purified by flash silica chromatography, elution gradient 0 to 50% EtOAc in Et₂O to give the title compound (0.26 g, 80%) as a yellow solid. ¹H NMR δ −0.22 (9H, s), 0.64-0.83 (2H, m), 1.55-1.80 (8H, m), 2.50-2.57 (3H, m), 2.70-2.77 (3H, m), 3.10-3.20 (2H, m), 3.51-3.61 (2H, m), 3.63-3.72 (2H, m), 3.75-3.81 (2H, m), 3.82-3.89 (2H, m), 4.73 (1H, t), 5.99 (2H, s), 6.69-6.87 (2H, m), 6.94-7.05 (1H, m), 7.03-7.16 (2H, m), 7.16 (1H, d); m/z: ES⁺ [M+H]⁺=577.4.

### Intermediate 148h

### 7-(4-{4-[3-(1,3-Dioxolan-2-yl)propoxy]phenyl}piperidin-1-yl)-4-methyl-1H-indazole-3-carbonitrile

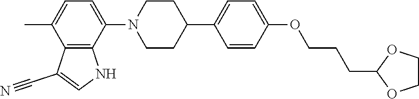 [SMILES: Cc1ccc(N2CCC(c3ccc(OCCCC4OCCO4)cc3)CC2)c2[nH]cc(C#N)c12]

The title compound was prepared using methodology described in intermediate 27g using 7-(4-{4-[3-(1,3-fioxolan-2-yl)propoxy]phenyl}piperidin-1-yl)-4-methyl-1-{[2-(trimethylsilyl)ethoxy]methyl}-1H-indazole-3-carbonitrile to give the title compound (120 mg, 67%) as a white solid. m/z: ES⁺ [M+H]⁺=447.2.

### Example 148

### 7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indazole-3-carbonitrile

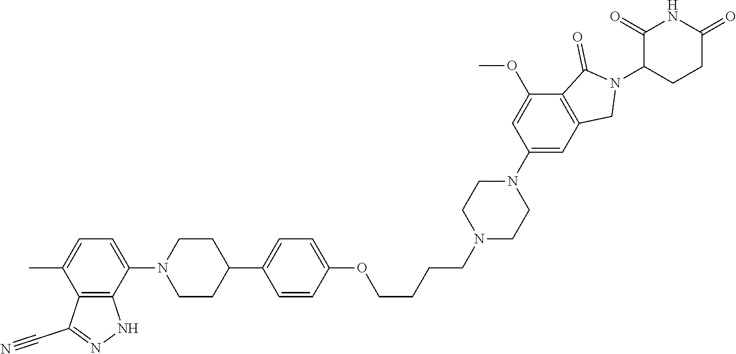 [SMILES: COc1cc(N2CCN(CCCCOc3ccc(C4CCN(c5ccc(C)c6c(C#N)n[nH]c56)CC4)cc3)CC2)cc2c1C(=O)N(C1CCC(=O)NC1=O)C2]

The title compound was prepared using methodology described in example 24 using 7-(4-{4-[3-(1,3-dioxolan-2-yl)propoxy]phenyl}piperidin-1-yl)-4-methyl-1H-indazole-3-carbonitrile and intermediate 7c to give the title compound (16 mg, 9%) as a white solid. ¹H NMR δ 1.63 (2H, q), 1.76 (2H, t), 1.82-2.01 (5H, m), 2.40 (2H, t), 2.58 (1H, s), 2.63 (3H, t), 2.80 (2H, q), 2.83-2.96 (2H, m), 3.29 (4H, d), 3.34 (5H, s), 3.51 (2H, d), 3.84 (3H, d), 4.00 (2H, dt), 4.10 (1H, d), 4.23 (1H, d), 4.92-5.01 (1H, m), 6.49 (1H, s), 6.61 (1H, s), 6.85-7.03 (4H, m), 7.21 (2H, dd), 10.91 (1H, s), 14.28 (1H, s); m/z: ES⁺ [M+H]⁺=745.4.

### Example 149

### Intermediate 149a

### 7-(4-{4-[4-(1,3-Dioxolan-2-yl)piperidin-1-yl]phenyl}piperidin-1-yl)-4-methyl-1H-indazole-3-carbonitrile

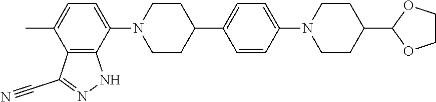 [SMILES: Cc1ccc(N2CCC(c3ccc(N4CCC(C5OCCO5)CC4)cc3)CC2)c2[nH]nc(C#N)c12]

The title compound was prepared using methodology described in intermediate 23a using intermediate 148c and intermediate 1e to give the title compound (90 mg, 28%) as a yellow solid. ¹H NMR δ 1.30-1.48 (2H, m), 1.54-1.65 (1H, m), 1.67-1.77 (2H, m), 1.83-1.89 (2H, m), 1.90-2.00 (2H, m), 2.49-2.63 (7H, m), 2.73-2.82 (1H, m), 3.43-3.53 (2H, m), 3.61-3.71 (2H, m), 3.71-3.92 (4H, m), 4.60 (1H, d), 6.82-6.93 (3H, m), 6.99 (1H, d), 7.11 (2H, d), 14.25 (1H, s); m/z: ES⁺ [M+H]⁺=472.3.

### Example 149

### 7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indazole-3-carbonitrile

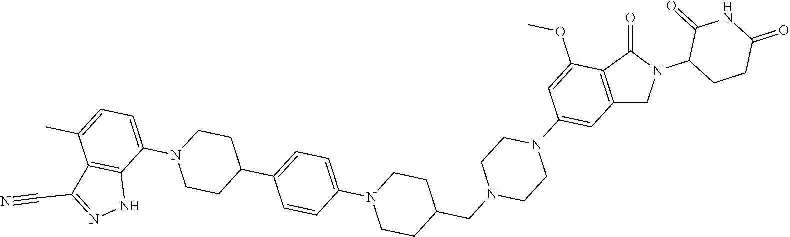 [SMILES: COc1cc(N2CCN(CC3CCN(c4ccc(C5CCN(c6ccc(C)c7c(C#N)n[nH]c67)CC5)cc4)CC3)CC2)cc2c1C(=O)N(C1CCC(=O)NC1=O)C2]

7-(4-{4-[4-(1,3-Dioxolan-2-yl)piperidin-1-yl]phenyl}piperidin-1-yl)-4-methyl-1H-indazole-3-carbonitrile (80 mg, 0.17 mmol) was stirred in formic acid (2 mL) at 60° C. for 3 h. The solvent was evaporated to give a residue.

Intermediate 7c (70.7 mg, 0.15 mmol) was also stirred in formic acid (2 mL) at 40° C. for 1 h. The solvent was evaporated to give a residue. The two residues were combined in NMP (2 mL) and stirred at RT for 16 h. The reaction mixture was diluted with DCM and washed with NaHCO₃ solution. The organic layer was evaporated to afford crude product. The crude product was purified by flash silica chromatography, elution gradient 0 to 10% MeOH in DCM to give a yellow solid. The yellow solid was purified flash C18-flash chromatography, elution gradient 0 to 100% MeCN in water (0.1% formic acid). Pure fractions were evaporated to dryness to give the title compound in the form of a formate salt (9.3 mg, 3.5%) as a yellow solid. ¹H NMR δ 1.16-1.27 (2H, m), 1.67-1.71 (1H, m), 1.77-2.02 (7H, m), 2.20-2.38 (3H, m), 2.54 (4H, s), 2.56-2.68 (6H, m), 2.72-2.82 (2H, m), 2.82-2.95 (2H, m), 3.27-3.32 (4H, m), 3.47-3.54 (2H, m), 3.61-3.68 (2H, m), 3.83 (3H, s), 4.10 (1H, d), 4.23 (1H, d), 4.96 (1H, dd), 6.49 (1H, d), 6.61 (1H, s), 6.85-6.94 (3H, m), 7.00 (1H, d), 7.10-7.17 (2H, m), 10.91 (1H, s), 14.15 (1H, s); m/z: ES⁺ [M+H]⁺=770.5.

### Example 150

### Intermediate 150a

### Benzyl 3-(4-bromophenyl)pyrrolidine-1-carboxylate

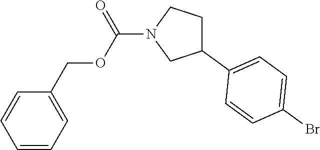 [SMILES: O=C(OCc1ccccc1)N1CCC(c2ccc(Br)cc2)C1]

The title compound was prepared using methodology described in intermediate 60a using 3-(4-bromophenyl)pyrrolidine and benzyl carbonchloridate to give the title compound (4.4 g, 92%) as a colourless gum. ¹H NMR (CDCl₃) δ 1.94-2.09 (1H, m), 2.27-2.35 (1H, m), 3.31-3.41 (2H, m), 3.45-3.56 (1H, m), 3.68-3.72 (1H, m), 3.88-3.93 (1H, m), 5.19 (2H, s), 7.08-7.16 (2H, m), 7.29-7.47 (5H, m), 7.43-7.50 (2H, m); m/z: ES⁺ [M+H]⁺=360.1.

### Intermediate 150b

### Benzyl 3-{4-[3-(1,3-dioxolan-2-yl)propoxy]phenyl}pyrrolidine-1-carboxylate

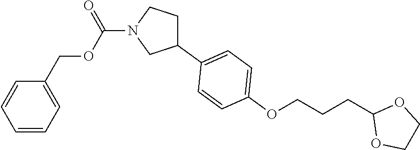 [SMILES: O=C(OCc1ccccc1)N1CCC(c2ccc(OCCCC3OCCO3)cc2)C1]

RockPhos Pd G3 (0.465 g, 0.56 mmol) was added to benzyl 3-(4-bromophenyl)pyrrolidine-1-carboxylate (2.0 g, 5.55 mmol), 1,3-dioxolane-2-propanol (1.29 g, 8.33 mmol), Cs₂CO₃ (3.62 g, 11.10 mmol) and RockPhos (0.260 g, 0.56 mmol) in toluene (30 mL) under nitrogen. The resulting mixture was stirred at 100° C. for 16 h. The reaction was cooled to RT and the solvent was evaporated to dryness to afford crude product. The crude product was purified by flash silica chromatography, elution gradient 0 to 40% EtOAc in Et₂O to give the title compound (1.0 g, 43.8%) as a colourless gum. ¹H NMR (CDCl₃) δ 1.82-1.93 (3H, m), 1.92-2.09 (3H, m), 2.25-2.29 (1H, m), 3.31-3.44 (2H, m), 3.44-3.53 (1H, m), 3.63-3.79 (1H, m), 3.82-4.07 (6H, m), 4.96 (1H, t), 5.19 (2H, d), 6.87 (2H, d), 7.15 (2H, d), 7.30-7.43 (5H, m); m/z: ES⁺ [M+H]⁺=412.2.

### Intermediate 150c: 3-{4-[3-(1,3-Dioxolan-2-yl)propoxy]phenyl}pyrrolidine

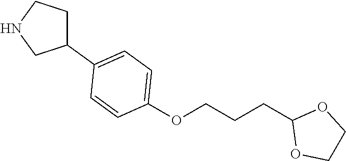 [SMILES: c1cc(C2CCNC2)ccc1OCCCC1OCCO1]

Pd/C (10%, 0.517 g, 0.49 mmol) was added to benzyl 3-{4-[3-(1,3-dioxolan-2-yl)propoxy]phenyl}pyrrolidine-1-carboxylate (1.0 g, 2.43 mmol) in MeOH (10 mL) under hydrogen. The resulting mixture was stirred at RT for 3 h. The precipitate was collected by filtration, washed with DCM (50 mL) and dried in the vacuum oven to give the title compound (0.630 g, 93%) as a yellow solid, which was used without further purification. ¹H NMR (CDCl₃) δ 1.19-1.31 (1H, m), 1.74-1.89 (2H, m), 1.84-1.98 (3H, m), 2.02 (1H, d), 2.14-2.31 (1H, m), 2.84 (1H, t), 3.06-3.28 (1H, m), 3.29-3.44 (1H, m), 3.66-4.05 (7H, m), 4.94 (1H, t), 6.78-6.89 (2H, m), 7.1-7.18 (2H, m); m/z: ES⁺ [M+H]⁺=278.0.

### Intermediates 150d and intermediate 151a

The title compound was prepared using methodology described in intermediate 11c using 3-{4-[3-(1,3-dioxolan-2-yl)propoxy]phenyl}pyrrolidine and intermediate 8a to give the title compound as a racemic mixture. The enantiomers were separated by preparative chiral-HPLC on a Column: CHIRALPAKIA-3 4.6*50 mm 3 μm; Flow rate: 1 mL/min; Mobile Phase A: Hex (0.1% DEA), Mobile Phase B: EtOH (1:1); to give, in order of elution, intermediate 150d (isomer 1, 95 mg, 50%, >99% ee) and intermediate 151a (isomer 2, 90 mg, 47%, >99% ee).

### Intermediate 150d

### 7-[(3S*)-3-{4-[3-(1,3-Dioxolan-2-yl)propoxy]phenyl}pyrrolidin-1-yl]-4-methyl-1H-indole-3-carbonitrile [Absolute Stereochemistry not Yet Confirmed]

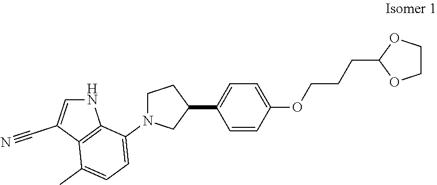

### Intermediate 151a

### 7-[(3R*)-3-{4-[3-(1,3-Dioxolan-2-yl)propoxy]phenyl}pyrrolidin-1-yl]-4-methyl-1H-indole-3-carbonitrile [Absolute Stereochemistry not Yet Confirmed]

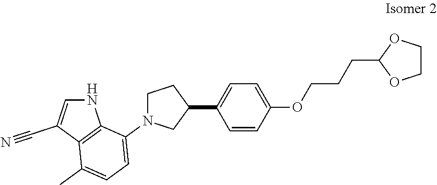

¹H NMR δ 1.74-1.8 (9H, m), 2.36 (1H, s), 2.55 (3H, s), 3.56-3.63 (1H, m), 3.69-3.8 (3H, m), 3.87-3.91 (1H, m), 3.93-4.01 (2H, m), 4.85 (1H, t), 6.43 (1H, d), 6.79 (1H, d), 6.89 (2H, d), 7.28 (2H, d), 8.07 (1H, d), 11.59 (1H, s).

### Example 150

### 7-{(3S*)-3-[4-(4-{4-[2-(2,6-Dioxopiperidin-3-yl)-1-oxo-2,3-dihydro-1H-isoindol-5-yl]piperazin-1-yl}butoxy)phenyl]pyrrolidin-1-yl}-4-methyl-1H-indole-3-carbonitrile [Absolute Stereochemistry not Yet Confirmed]

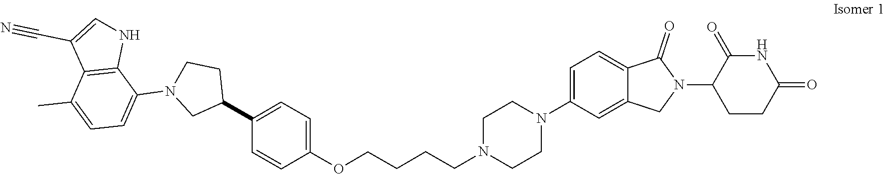

The title compound was prepared using methodology described in example 24 using intermediate 150d and intermediate 1i to give the title compound (6 mg, 4%) as a white solid. ¹H NMR δ 1.59-1.64 (2H, m), 1.7-1.79 (2H, m), 1.9-2.05 (2H, m), 2.31-2.43 (5H, m), 2.55 (4H, s), 2.59 (1H, s), 2.69 (1H, d), 2.88 (1H, d), 3.27 (5H, d), 3.45 (3H, s), 3.60 (1H, d), 3.71 (1H, s), 3.98 (2H, t), 4.19 (1H, d), 4.32 (1H, d), 5.05 (1H, dd), 6.42 (1H, d), 6.79 (1H, d), 6.90 (2H, d), 7.05 (2H, d), 7.28 (2H, d), 7.52 (1H, d), 8.08 (1H, d), 10.95 (1H, s), 11.60 (1H, s); m/z: ES⁺ [M+H]⁺=700.4.

### Example 151

### 7-{(3R*)-3-[4-(4-{4-[2-(2,6-Dioxopiperidin-3-yl)-1-oxo-2,3-dihydro-1H-isoindol-5-yl]piperazin-1-yl}butoxy)phenyl]pyrrolidin-1-yl}-4-methyl-1H-indole-3-carbonitrile [Absolute Stereochemistry not Yet Confirmed]

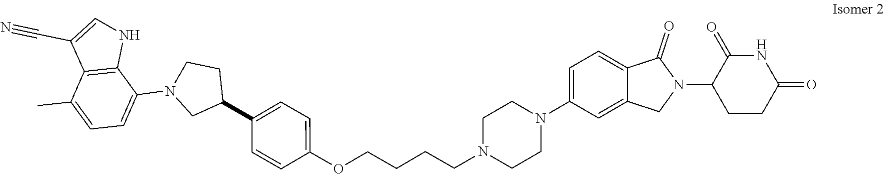

The title compound was prepared using methodology described in example 24 using intermediate 151a and intermediate 1i to give the title compound (12 mg, 8%) as a white solid. ¹H NMR δ 1.59-1.64 (2H, m), 1.7-1.79 (2H, m), 1.9-2.05 (2H, m), 2.31-2.43 (5H, m), 2.55 (4H, s), 2.59 (1H, s), 2.69 (1H, d), 2.88 (1H, d), 3.27 (5H, d), 3.45 (3H, s), 3.60 (1H, d), 3.71 (1H, s), 3.98 (2H, t), 4.19 (1H, d), 4.32 (1H, d), 5.05 (1H, dd), 6.42 (1H, d), 6.79 (1H, d), 6.90 (2H, d), 7.05 (2H, d), 7.28 (2H, d), 7.52 (1H, d), 8.08 (1H, d), 10.95 (1H, s), 11.60 (1H, s); m/z: ES⁺ [M+H]⁺=700.4.

### Example 152

### Intermediate 152a

### Methyl 4-[4-(dibutoxymethyl)piperidin-1-yl]-2-fluorobenzoate

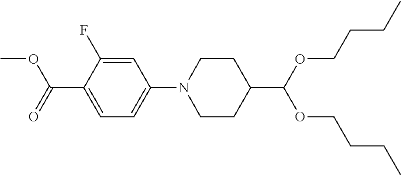 [SMILES: CCCCOC(OCCCC)C1CCN(c2ccc(C(=O)OC)c(F)c2)CC1]

The title compound was prepared using methodology described in intermediate 11a using methyl 4-bromo-2-fluorobenzoate and 4-(dibutoxymethyl)piperidine to give the title compound (3.0 g, 35%) as a pale yellow oil. ¹H NMR δ 0.88 (6H, t), 1.21-1.38 (6H, m), 1.48 (4H, m), 1.70 (2H, d), 1.81 (1H, m), 2.77-2.88 (2H, m), 3.38 (2H, dt), 3.55 (2H, dt), 3.79 (3H, s), 3.94 (2H, d), 4.17 (1H, d), 6.67-6.79 (2H, m), 7.68 (1H, t); m/z: ES⁺ [M+H]⁺=396.1.

### Intermediate 152b

### 4-[4-(Dibutoxymethyl)piperidin-1-yl]-2-fluorobenzoic acid

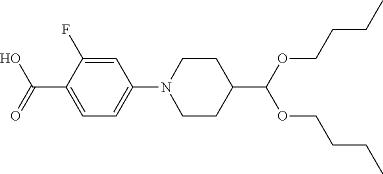 [SMILES: CCCCOC(OCCCC)C1CCN(c2ccc(C(=O)O)c(F)c2)CC1]

Methyl 4-[4-(dibutoxymethyl)piperidin-1-yl]-2-fluorobenzoate (900 mg, 2.28 mmol) was added to sodium hydroxide (546 mg, 13.65 mmol) in water (5 mL) and MeOH (5 mL) at 25° C. The mixture was stirred at 50° C. for 2.5 h. The reaction mixture was cooled to RT and acidified with HCl (2M), filtered and evaporated to give the title compound (570 mg, 63.3%) as a white solid which was used without further purification. ¹H NMR δ 0.89 (6H, d), 1.23-1.39 (7H, m), 1.48 (5H, dq), 1.66-1.74 (2H, m), 1.81 (1H, dtd), 2.81 (2H, td), 3.52-3.56 (2H, m), 3.92 (2H, d), 4.17 (1H, d), 6.61-6.79 (2H, m), 7.67 (1H, t), 12.36 (1H, s); m/z: ES⁺ [M+H]⁺=382.2.

### Intermediate 152c

### 4-Chloro-7-(1,4-dioxa-8-azaspiro[4.5]decan-8-yl)-1H-indazole-3-carbonitrile

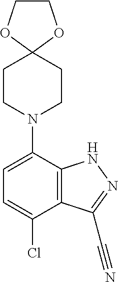 [SMILES: N#Cc1n[nH]c2c(N3CCC4(CC3)OCCO4)ccc(Cl)c12]

The title compound was prepared using methodology described in intermediate 11c using intermediate 9c and 1,4-dioxa-8-azaspiro[4.5]decane to give the title compound (1.0 g, 53%) as a yellow solid. ¹H NMR δ 1.82-2.03 (4H, m), 3.15 (4H, t), 4.02 (4H, d), 6.95 (1H, d), 7.34 (1H, dd), 14.67 (1H, s); m/z: ES⁻[M−H]⁻=317.1.

### Intermediate 152d

### 4-Chloro-7-(4-oxopiperidin-1-yl)-1H-indazole-3-carbonitrile

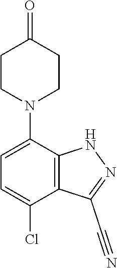 [SMILES: N#Cc1n[nH]c2c(N3CCC(=O)CC3)ccc(Cl)c12]

4-Chloro-7-(1,4-dioxa-8-azaspiro[4.5]decan-8-yl)-1H-indazole-3-carbonitrile (1.0 g, 3.14 mmol) was added to formic acid (13 mL) and was stirred at 40° C. for 16 h. The reaction was cooled to RT and the solvent was evaporated to afford crude product. The crude product was purified by flash silica chromatography, elution gradient 0 to 50% EtOAc in Et₂O to give the title compound (0.580 g, 67.5%) as a brown solid. ¹H NMR S 2.64 (4H, t), 3.43 (4H, t), 7.01 (1H, d), 7.33 (1H, d), 14.80 (1H, s); m/z: ES⁺ [M+H]⁺=275.1.

### Intermediate 152e

### 7-(4-Aminopiperidin-1-yl)-4-chloro-1H-indazole-3-carbonitrile

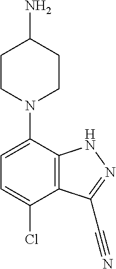 [SMILES: N#Cc1n[nH]c2c(N3CCC(N)CC3)ccc(Cl)c12]

Sodium cyanoborohydride (320 mg, 5.10 mmol) was added to ammonium acetate (1.5 g, 20.39 mmol) and 4-chloro-7-(4-oxopiperidin-1-yl)-1H-indazole-3-carbonitrile (700 mg, 2.55 mmol) in MeOH (10 mL). The mixture was stirred at 25° C. for 2 h. The solvent was evaporated to afford crude product. The crude product was purified by flash C18-flash chromatography, elution gradient 0 to 30% MeOH in water (0.2% HCl) to give the title compound (400 mg, 56.9%) as a pale yellow solid. 1H NMR δ 1.75-2.1 (3H, m), 2.79 (1H, td), 3.50 (1H, d), 4.27 (6H, s), 6.93 (1H, d), 7.31 (1H, d), 14.82 (1H, s); m/z: ES⁺ [M+H]⁺=276.2.

### Intermediate 152f

### N-[1-(4-Chloro-3-cyano-1H-indazol-7-yl)piperidin-4-yl]-4-[4-(dibutoxymethyl)piperidin-1-yl]-2-fluorobenzamide

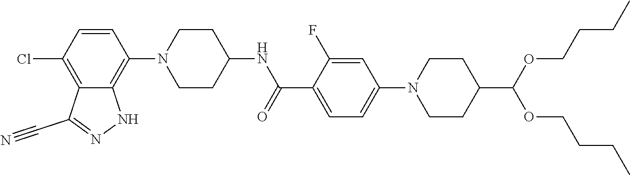 [SMILES: CCCCOC(OCCCC)C1CCN(c2ccc(C(=O)NC3CCN(c4ccc(Cl)c5c(C#N)n[nH]c45)CC3)c(F)c2)CC1]

DIPEA (0.190 mL, 1.09 mmol) and HATU (138 mg, 0.36 mmol) were added to 4-[4-(dibutoxymethyl)piperidin-1-yl]-2-fluorobenzoic acid (166 mg, 0.44 mmol) and 7-(4-aminopiperidin-1-yl)-4-chloro-1H-indazole-3-carbonitrile (100 mg, 0.36 mmol) in DMF (2 mL). The resulting mixture was stirred at 40° C. for 1 h. The reaction was cooled to RT and was diluted with EtOAc (15 mL), washed with water (15 mL), saturated brine (20 mL), dried over Na₂SO₄, filtered and evaporated to afford crude product. The crude product was purified by flash silica chromatography, elution gradient 0 to 50% EtOAc in Et₂O to give the title compound (110 mg, 47.5%) as a yellow gum; ¹H NMR δ 0.89 (6H, t), 1.33 (6H, dt), 1.43-1.59 (5H, m), 1.71 (3H, d), 1.81-2.04 (5H, m), 2.85 (3H, dd), 3.45-3.61 (4H, m), 3.8-3.95 (3H, m), 4.14-4.22 (1H, m), 5.77 (1H, s), 6.63-6.84 (2H, m), 6.95 (1H, d), 7.31 (1H, d), 7.51 (1H, t), 7.69-7.9 (1H, m), 14.70 (1H, s); m/z: ES⁺ [M+H]⁺=639.1.

### Example 152

### N-[1-(4-Chloro-3-cyano-1H-indazol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide

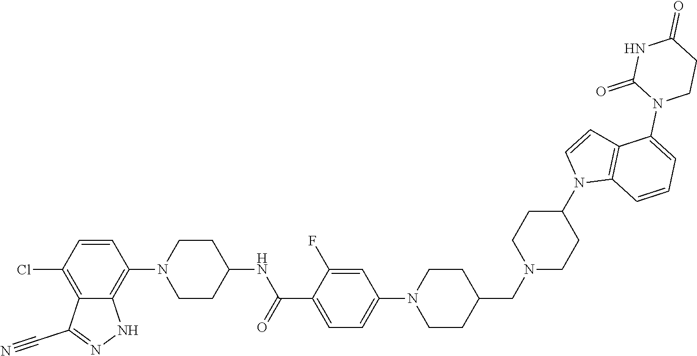 [SMILES: N#Cc1n[nH]c2c(N3CCC(NC(=O)c4ccc(N5CCC(CN6CCC(n7ccc8c(N9CCC(=O)NC9=O)cccc87)CC6)CC5)cc4F)CC3)ccc(Cl)c12]

The title compound was prepared using methodology described in example 140 using N-[1-(4-chloro-3-cyano-1H-indazol-7-yl)piperidin-4-yl]-4-[4-(dibutoxymethyl)piperidin-1-yl]-2-fluorobenzamide and intermediate 5f to give the title compound in the form of a formate salt (28 mg, 25%) as a white solid. ¹H NMR δ 0.86 (1H, d), 1.17 (2H, d), 1.84 (5H, t), 1.99 (6H, d), 2.09-2.29 (4H, m), 2.73-2.89 (5H, m), 3.01 (2H, d), 3.52 (2H, s), 3.78 (2H, t), 3.83-3.99 (3H, m), 4.39 (1H, s), 6.43 (1H, d), 6.77 (2H, dd), 6.90 (1H, d), 6.97 (1H, d), 7.15 (1H, t), 7.28 (1H, d), 7.45-7.63 (3H, m), 7.72-7.92 (1H, m), 10.36 (1H, s) 1H not observed; m/z: ES⁺ [M+H]⁺=805.3.

### Example 153

### Intermediate 153a

### 4-Chloro-7-(1,4-dioxa-8-azaspiro[4.5]decan-8-yl)-1H-indole-3-carbonitrile

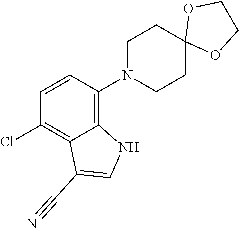 [SMILES: N#Cc1c[nH]c2c(N3CCC4(CC3)OCCO4)ccc(Cl)c12]

The title compound was prepared using methodology described in intermediate 11c using intermediate 1b and 1,4-dioxa-8-azaspiro[4.5]decane to give the title compound (1.9 g, 46%) as a white solid. ¹H NMR δ 1.88 (4H, t), 3.05 (4H, dd), 3.93 (4H, s), 6.84 (1H, d), 7.14 (1H, d), 8.33 (1H, d), 12.28 (1H, s); m/z: ES⁺ [M+H]⁺=318.2.

### Intermediate 153b

### 4-Chloro-7-(4-oxopiperidin-1-yl)-1H-indole-3-carbonitrile

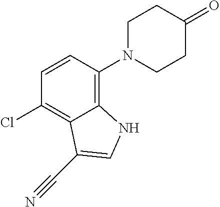 [SMILES: N#Cc1c[nH]c2c(N3CCC(=O)CC3)ccc(Cl)c12]

The title compound was prepared using methodology described in intermediate 152d using 4-chloro-7-(1,4-dioxa-8-azaspiro[4.5]decan-8-yl)-1H-indole-3-carbonitrile to give the title compound (1.6 g, 98%) as a white solid. ¹H NMR (CDCl₃) δ 2.72 (4H, t), 3.41 (4H, t), 6.95 (1H, d), 7.21 (1H, d), 7.84 (1H, d), 9.24 (1H, s); m/z: ES⁺ [M+H]⁺=274.2.

### Intermediate 153c

### 4-Chloro-7-[4-(hydroxyimino)piperidin-1-yl]-1H-indole-3-carbonitrile

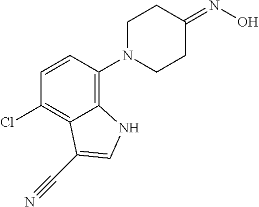 [SMILES: N#Cc1c[nH]c2c(N3CCC(=NO)CC3)ccc(Cl)c12]

Sodium acetate (1.34 g, 16.37 mmol) was added to 4-chloro-7-(4-oxopiperidin-1-yl)-1H-indole-3-carbonitrile (1.6 g, 5.85 mmol) and hydroxylammonium chloride solution (1.54 g, 22.21 mmol) in MeOH (25 mL) and was stirred at 25° C. for 16 h. The solvent was evaporated to afford crude product. The crude product was purified by flash silica chromatography, elution gradient 0 to 50% EtOAc in Et₂O to give the title compound (1.6 g, 95%) as a yellow solid. ¹H NMR δ 2.48 (1H, d), 2.75 (2H, t), 3.03 (2H, t), 3.10 (2H, t), 3.37 (1H, s), 6.84 (1H, d), 7.15 (1H, d), 8.36 (1H, s), 10.44 (1H, s), 12.36 (1H, s); m/z: ES⁻[M−H]⁻=287.1.

### Intermediate 153d

### 7-(4-Aminopiperidin-1-yl)-4-chloro-1H-indole-3-carbonitrile

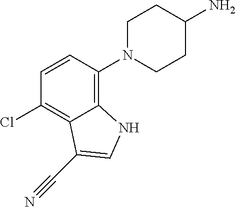 [SMILES: N#Cc1c[nH]c2c(N3CCC(N)CC3)ccc(Cl)c12]

LiAlH₄ (7.18 ml, 17.94 mmol) was added to 4-chloro-7-[4-(hydroxyimino)piperidin-1-yl]-1H-indole-3-carbonitrile (1.4 g, 4.85 mmol) in THF (35 mL) and was stirred at 25° C. for 4 h. The reaction mixture was diluted with EtOAc (100 mL), washed with sodium potassium tartrate (150 mL), dried over Na₂SO₄, filtered and evaporated to afford crude product. The crude product was purified by flash C18-flash chromatography, elution gradient 0 to 30% MeOH in water (0.1% NH₄HCO₃). Pure fractions were evaporated to dryness to afford 7-(4-aminopiperidin-1-yl)-4-chloro-1H-indole-3-carbonitrile (0.900 g, 67.6%) as a yellow solid. ¹H NMR δ 1.54 (1H, d), 1.61 (1H, d), 1.82 (2H, t), 2.6-2.82 (3H, m), 3.34 (2H, d), 6.76 (1H, d), 7.09 (1H, d), 8.25 (1H, s); m/z: ES⁺ [M+H]⁺=273.1.

### Intermediate 153e

### N-[1-(4-Chloro-3-cyano-1H-indol-7-yl)piperidin-4-yl]-4-[4-(dibutoxymethyl)piperidin-1-yl]-2-fluorobenzamide

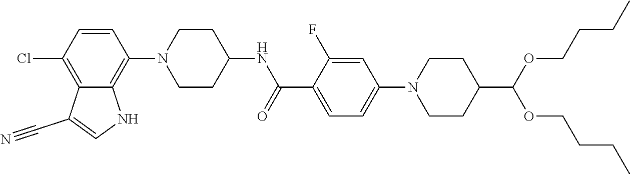 [SMILES: CCCCOC(OCCCC)C1CCN(c2ccc(C(=O)NC3CCN(c4ccc(Cl)c5c(C#N)c[nH]c45)CC3)c(F)c2)CC1]

HATU (120 mg, 0.31 mmol) was added to intermediate 152b (80 mg, 0.21 mmol), 7-(4-aminopiperidin-1-yl)-4-chloro-1H-indole-3-carbonitrile (86 mg, 0.31 mmol) and DIPEA (0.073 mL, 0.42 mmol) in THF (2 mL) and was stirred at RT for 2 h. The mixture was diluted with EtOAc (10 mL), washed with water (10 mL), saturated brine (10 mL) dried over Na₂SO₄, filtered and evaporated to afford crude product. The crude product was purified by flash silica chromatography, elution gradient 0 to 60% EtOAc in Et₂O to give the title compound (100 mg, 74.7%) as a yellow solid. ¹H NMR δ 0.89 (7H, t), 1.19-1.36 (20H, m), 1.37 (2H, d), 1.48 (4H, q), 1.71 (2H, d), 1.93 (4H, s), 2.78 (4H, d), 3.15 (1H, dd), 3.41 (1H, s), 3.49-3.68 (3H, m), 3.87 (3H, d), 4.18 (1H, d), 6.67-6.90 (3H, m), 7.16 (1H, d), 7.50 (1H, t), 7.80 (1H, s), 8.32 (1H, d), 12.30 (1H, s); m/z: ES⁺ [M+H]⁺=638.0.

### Example 153

### N-[1-(4-Chloro-3-cyano-1H-indol-7-yl)piperidin-4-yl]-4-[4-({4-[5-(2,4-dioxo-1,3-diazinan-1-yl)-1H-indol-1-yl]piperidin-1-yl}methyl)piperidin-1-yl]-2-fluorobenzamide

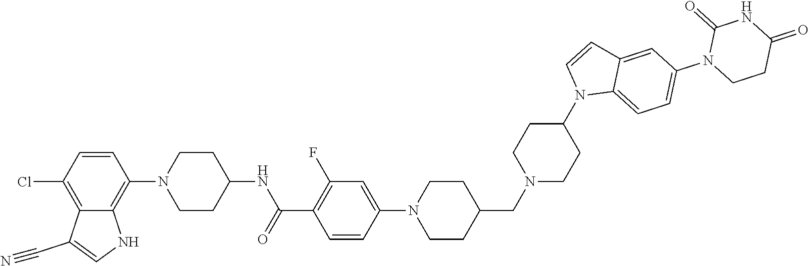 [SMILES: N#Cc1c[nH]c2c(N3CCC(NC(=O)c4ccc(N5CCC(CN6CCC(n7ccc8cc(N9CCC(=O)NC9=O)ccc87)CC6)CC5)cc4F)CC3)ccc(Cl)c12]

The title compound was prepared using methodology described in example 117 using N-[1-(4-chloro-3-cyano-1H-indol-7-yl)piperidin-4-yl]-4-[4-(dibutoxymethyl)piperidin-1-yl]-2-fluorobenzamide and intermediate 89c to give the title compound (17 mg, 13%) as a white solid. 1H NMR δ 1.17 (2H, d), 1.67-1.88 (5H, m), 1.89-2.08 (6H, m), 2.11-2.31 (4H, m), 2.65-2.87 (6H, m), 3.00 (2H, d), 3.49 (2H, s), 3.78 (2H, t), 3.87 (3H, d), 4.38 (1H, s), 6.42 (1H, d), 6.65-6.84 (3H, m), 6.97 (1H, d), 7.06 (1H, d), 7.15 (1H, t), 7.4-7.62 (3H, m), 7.78 (1H, d), 8.20 (1H, s), 10.34 (1H, s), 12.32 (1H, s); m/z: ES⁺ [M+H]⁺=804.5.

### Example 154

### N-[1-(4-chloro-3-cyano-1~{H}-indol-7-yl)-4-piperidyl]-4-[4-[[4-[(2,6-dioxo-3-piperidyl)carbamoyl]-3-fluoro-phenyl]piperazin-1-yl]methyl]-1-piperidyl]-2-fluorobenzamide

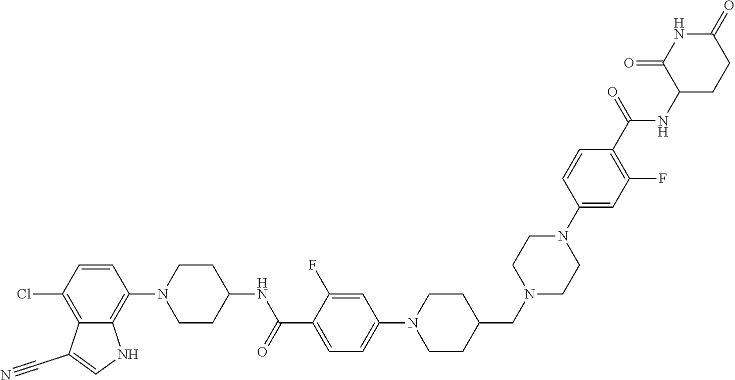 [SMILES: N#Cc1c[nH]c2c(N3CCC(NC(=O)c4ccc(N5CCC(CN6CCN(c7ccc(C(=O)NC8CCC(=O)NC8=O)c(F)c7)CC6)CC5)cc4F)CC3)ccc(Cl)c12]

The title compound was prepared using methodology described in example 128 using intermediate 153e and intermediate 104c to give the title compound in the form of a formate salt (54 mg, 26%) as a white solid. ¹H NMR δ 1.15 (2H, q), 1.73-2.02 (8H, m), 2.06-2.14 (1H, m), 2.19 (2H, d), 2.47 (4H, t), 2.54 (4H, s), 2.72-2.87 (5H, m), 3.31-3.4 (3H, m), 3.87 (3H, t), 4.68-4.78 (1H, m), 6.7-6.88 (5H, m), 7.16 (1H, d), 7.50 (1H, t), 7.63 (1H, t), 7.81 (1H, dd), 8.02-8.09 (1H, m), 8.16 (1H, s), 8.32 (1H, s), 10.86 (1H, s), 12.32 (1H, s); m/z: ES⁺ [M+H]⁺=824.2.

### Example 155

### N-[1-(4-Chloro-3-cyano-1H-indol-7-yl)piperidin-4-yl]-4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)-2-fluorobenzamide

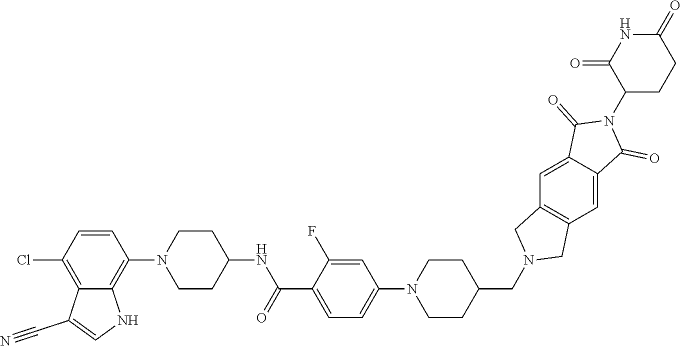 [SMILES: N#Cc1c[nH]c2c(N3CCC(NC(=O)c4ccc(N5CCC(CN6Cc7cc8c(cc7C6)C(=O)N(C6CCC(=O)NC6=O)C8=O)CC5)cc4F)CC3)ccc(Cl)c12]

The title compound was prepared using methodology described in example 128 using intermediate 153e and intermediate 75e to give the title compound in the form of a formate salt (34 mg, 23%) as a white solid. ¹H NMR δ 1.21 (3H, d), 1.76 (1H, s), 1.83 (5H, d), 1.94 (2H, s), 2.07 (2H, s), 2.57 (7H, d), 2.85 (51H, dq), 3.87 (4H, d), 3.97 (4H, s), 5.14 (1H, dd), 6.73 (1H, d), 6.81 (2H, dd), 7.14 (1H, d), 7.51 (1H, t), 7.79 (3H, s), 8.30 (1H, s), 11.12 (1H, s), 12.31 (1H, s); m/z: ES⁺ [M+H]⁺=791.3.

### Example 156

### N-[1-(4-Chloro-3-cyano-1H-indol-7-yl)piperidin-4-yl]-4-[4-({4-[6-(2,4-dioxo-1,3-diazinan-1-yl)-5-fluoro-1-methyl-1H-indol-2-yl]piperidin-1-yl}methyl)piperidin-1-yl]-2-fluorobenzamide

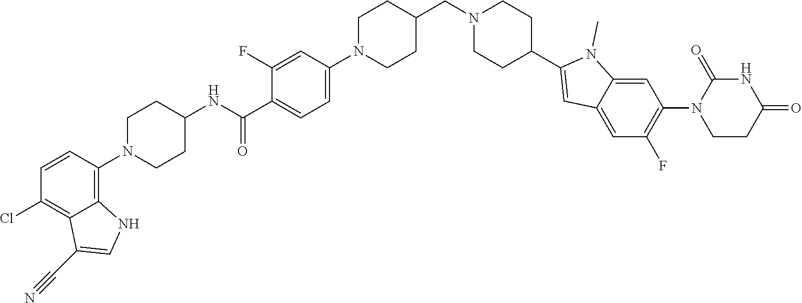 [SMILES: Cn1c(C2CCN(CC3CCN(c4ccc(C(=O)NC5CCN(c6ccc(Cl)c7c(C#N)c[nH]c67)CC5)c(F)c4)CC3)CC2)cc2cc(F)c(N3CCC(=O)NC3=O)cc21]

The title compound was prepared using methodology described in example 128 using intermediate 153e and intermediate 136g to give the title compound in the form of a formate salt (18 mg, 10%) as a white solid. ¹H NMR δ 1.15 (2H, dd), 1.65 (2H, dd), 1.75-1.87 (5H, m), 1.9-1.95 (4H, m), 2.01-2.11 (2H, m), 2.19 (2H, d), 2.7-2.87 (7H, m), 2.96 (2H, d), 3.51 (2H, s), 3.67-3.76 (5H, m), 3.8-3.93 (3H, m), 6.26 (1H, s), 6.68-6.82 (3H, m), 7.04 (1H, d), 7.30 (1H, d), 7.50 (2H, dd), 7.77 (1H, dd), 8.17 (1H, s), 10.41 (1H, s), 12.31 (1H, s); m/z: ES⁺ [M+H]⁺=836.3.

### Example 157

### N-[1-(4-Chloro-3-cyano-1H-indol-7-yl)piperidin-4-yl]-4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]-2-fluorobenzamide

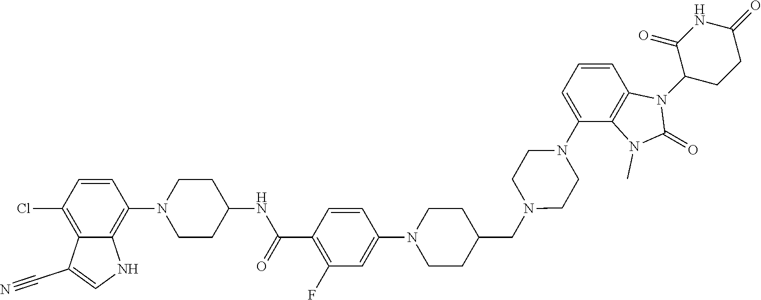 [SMILES: Cn1c(=O)n(C2CCC(=O)NC2=O)c2cccc(N3CCN(CC4CCN(c5ccc(C(=O)NC6CCN(c7ccc(Cl)c8c(C#N)c[nH]c78)CC6)c(F)c5)CC4)CC3)c21]

The title compound was prepared using methodology described in example 128 using intermediate 153e and intermediate 72b to give the title compound in the form of a formate salt (13 mg, 17%) as a white solid. ¹H NMR δ 1.07-1.31 (2H, m), 1.79 (3H, d), 1.91 (2H, d), 2-2.27 (3H, m), 2.50 (4H, q), 2.56-2.71 (2H, m), 2.78-3.24 (11H, m), 3.33 (1H, s), 3.34 (1H, s), 3.63 (3H, s), 3.88 (3H, dd), 6.59-7.01 (6H, m), 7.15 (1H, d), 7.51 (1H, t), 7.78 (1H, dd), 8.31 (1H, s), 11.09 (1H, s), 12.29 (1H, s) 1H not observed; m/z: ES⁺ [M+H]⁺=835.3.

### Example 158

### N-[1-(4-Chloro-3-cyano-1H-indol-7-yl)piperidin-4-yl]-4-[4-({4-[6-(2,4-dioxo-1,3-diazinan-1-yl)-1-methyl-1H-indol-2-yl]piperidin-1-yl}methyl)piperidin-1-yl]-2-fluorobenzamide

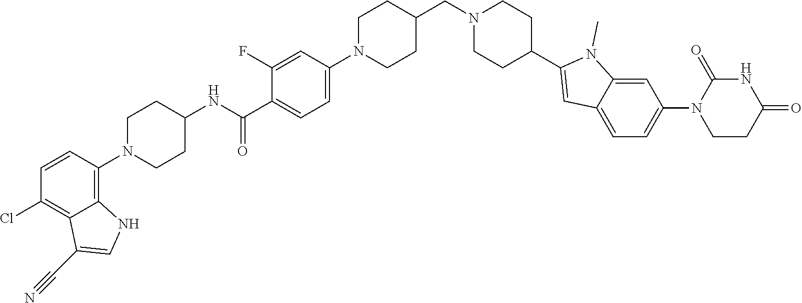 [SMILES: Cn1c(C2CCN(CC3CCN(c4ccc(C(=O)NC5CCN(c6ccc(Cl)c7c(C#N)c[nH]c67)CC5)c(F)c4)CC3)CC2)cc2ccc(N3CCC(=O)NC3=O)cc21]

The title compound was prepared using methodology described in example 128 using intermediate 153e and intermediate 13c to give the title compound in the form of a formate salt (22 mg, 8%) as a white solid. ¹H NMR δ 1.17 (2H, p), 1.65 (2H, tt), 1.71-2.03 (9H, m), 2.10 (2H, t), 2.22 (2H, d), 2.69-2.86 (7H, m), 2.98 (2H, d), 3.29-3.37 (2H, m), 3.68 (3H, s), 3.74-3.89 (2H, m), 3.91 (3H, d), 6.23 (1H, s), 6.63-6.86 (2H, m), 6.84 (1H, d), 6.93 (1H, dd), 7.15 (1H, d), 7.36 (1H, d), 7.43 (1H, d), 7.51 (1H, t), 7.78 (1H, dd), 8.31 (1H, s), 10.29 (1H, s), 12.31 (1H, s); m/z: ES⁺ [M+H]⁺=818.0.

### Example 159

### N-[1-(4-Chloro-3-cyano-1H-indol-7-yl)piperidin-4-yl]-4-[4-({4-[2-(2,6-dioxopiperidin-3-yl)-1-oxo-2,3-dihydro-1H-isoindol-5-yl]piperazin-1-yl}methyl)piperidin-1-yl]-2-fluorobenzamide

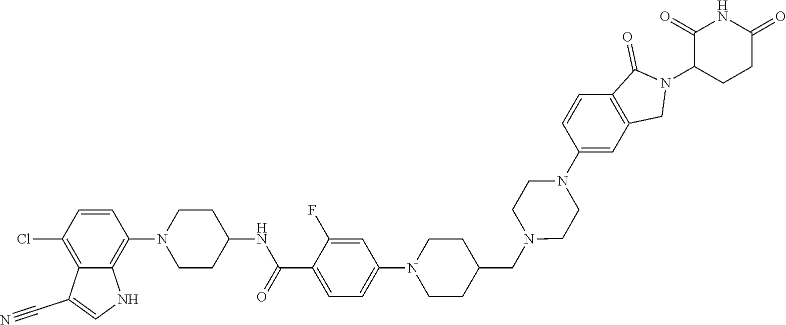 [SMILES: N#Cc1c[nH]c2c(N3CCC(NC(=O)c4ccc(N5CCC(CN6CCN(c7ccc8c(c7)CN(C7CCC(=O)NC7=O)C8=O)CC6)CC5)cc4F)CC3)ccc(Cl)c12]

The title compound was prepared using methodology described in example 128 using intermediate 153e and intermediate 1i to give the title compound in the form of a formate salt (17 mg, 14%) as a white solid. ¹H NMR δ 1.16 (3H, d), 1.7-2.01 (8H, m), 2.20 (2H, d), 2.35 (1H, s), 2.59 (2H, d), 2.80 (5H, s), 3.32 (7H, s), 3.85 (4H, d), 4.21 (1H, d), 4.33 (1H, d), 5.05 (1H, dd), 6.6-6.93 (3H, m), 7.06 (1H, d), 7.15 (1H, d), 7.51 (2H, dd), 7.78 (1H, d), 8.31 (2H, m), 10.96 (1H, s), 12.37 (1H, s); m/z: ES⁺ [M+H]⁺=820.4.

### Example 160

### N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide

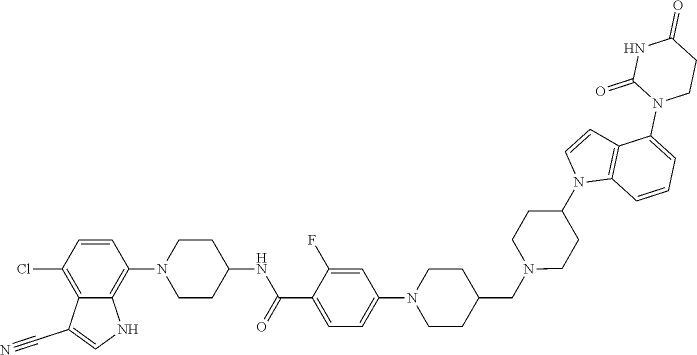 [SMILES: N#Cc1c[nH]c2c(N3CCC(NC(=O)c4ccc(N5CCC(CN6CCC(n7ccc8c(N9CCC(=O)NC9=O)cccc87)CC6)CC5)cc4F)CC3)ccc(Cl)c12]

The title compound was prepared using methodology described in example 117 using intermediate 153e and intermediate 5f to give the title compound in the form of a formate salt (23 mg, 18%) as a white solid. ¹H NMR δ 1.18 (2H, t), 1.65-1.92 (4H, m), 1.9-2.05 (7H, m), 2.1-2.31 (4H, m), 2.79 (6H, dt), 3.01 (2H, d), 3.32 (2H, s), 3.68-3.85 (2H, m), 3.88 (3H, s), 4.38 (1H, s), 6.42 (1H, d), 6.64-6.88 (3H, m), 6.96 (1H, d), 7.05-7.35 (2H, m), 7.38-7.58 (3H, m), 7.79 (1H, dd), 8.31 (1H, s), 10.34 (1H, s), 12.30 (1H, s); m/z: ES⁺ [M+H]=804.4.

### Example 161

### N-[1-(4-Chloro-3-cyano-1H-indol-7-yl)piperidin-4-yl]-4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]-2-fluorobenzamide

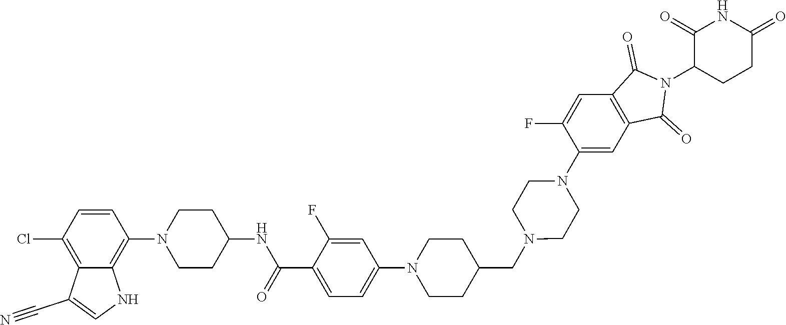 [SMILES: N#Cc1c[nH]c2c(N3CCC(NC(=O)c4ccc(N5CCC(CN6CCN(c7cc8c(cc7F)C(=O)N(C7CCC(=O)NC7=O)C8=O)CC6)CC5)cc4F)CC3)ccc(Cl)c12]

The title compound was prepared using methodology described in example 24 using intermediate 153e and intermediate 50b to give the title compound (335 mg, 16%) as a white solid. ¹H NMR δ 1.16 (2H, q), 1.73-2.04 (7H, m), 1.98-2.09 (1H, m), 2.21 (2H, d), 2.51-2.67 (5H, m), 2.75-3.01 (5H, m), 3.33 (7H, s), 3.88 (3H, dd), 5.11 (1H, dd), 6.67-6.96 (3H, m), 7.15 (1H, d), 7.43-7.66 (2H, m), 7.64-7.9 (2H, m), 8.31 (1H, s), 11.11 (1H, s), 12.27-12.32 (1H, m); m/z: ES⁺ [M+H]⁺=852.3.

### Example 162

### Intermediate 162a

### Methyl 2-cyano-4-[4-(dibutoxymethyl)piperidin-1-yl]benzoate

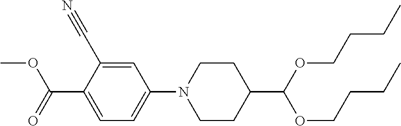 [SMILES: CCCCOC(OCCCC)C1CCN(c2ccc(C(=O)OC)c(C#N)c2)CC1]

The title compound was prepared using methodology described in intermediate 11a using methyl 4-bromo-2-cyanobenzoate and 4-(dibutoxymethyl)piperidine to give the title compound (1.6 g, 69%) as a white solid. ¹H NMR (CDCl₃) δ 0.95 (5H, t), 1.30 (OH, s), 1.34-1.44 (5H, m), 1.44 (1H, s), 1.59 (4H, dq), 1.90 (3H, q), 2.85-2.96 (2H, m), 3.45 (2H, dt), 3.65 (2H, dt), 3.93 (5H, d), 4.03 (1H, s), 4.19 (1H, d), 7.01 (1H, dd), 7.16 (1H, d), 7.97 (1H, d); m/z: ES⁺ [M+H]⁺=403.1.

### Intermediate 162b

### 2-Cyano-4-[4-(dibutoxymethyl)piperidin-1-yl]benzoic acid

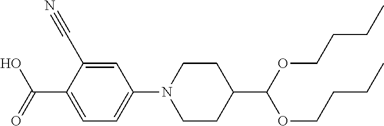 [SMILES: CCCCOC(OCCCC)C1CCN(c2ccc(C(=O)O)c(C#N)c2)CC1]

Lithium hydroxide (0.268 g, 11.18 mmol) was added to methyl 2-cyano-4-[4-(dibutoxymethyl)piperidin-1-yl]benzoate (1.5 g, 3.73 mmol) in THF (25 mL) and water (25 mL). The resulting solution was stirred at 25° C. for 1 h, followed by addition of HCl (1M, 11.18 mL, 11.18 mmol). The solvent was evaporated to give the title compound (0.90 g, 62.2%) as a yellow solid which was used in the next step without further purification. ¹H NMR δ 1.21 (1H, t), 1.91 (8H, qd), 2.88-2.96 (2H, m), 2.93-3.08 (2H, m), 3.13-3.26 (6H, m), 3.45 (5H, dd), 5.14-5.24 (2H, m), 7.28-7.47 (2H, m), 9.26 (4H, s); m/z: ES⁺ [M+H]⁺=389.1.

### Intermediate 162c

### N-[1-(4-Chloro-3-cyano-1H-indol-7-yl)piperidin-4-yl]-2-cyano-4-[4-(dibutoxymethyl)piperidin-1-yl]benzamide

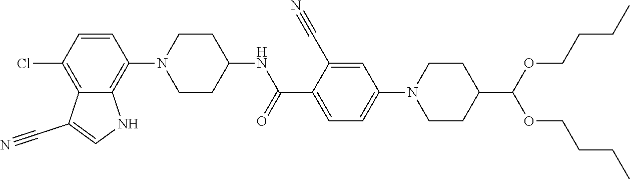 [SMILES: CCCCOC(OCCCC)C1CCN(c2ccc(C(=O)NC3CCN(c4ccc(Cl)c5c(C#N)c[nH]c45)CC3)c(C#N)c2)CC1]

EDC (148 mg, 0.77 mmol) was added to HOBt (118 mg, 0.77 mmol), DIPEA (0.135 mL, 0.77 mmol), 2-cyano-4-[4-(dibutoxymethyl)piperidin-1-yl]benzoic acid (150 mg, 0.39 mmol) and intermediate 153d (106 mg, 0.39 mmol) in DMF (2 mL) at 25° C. The solution was stirred at 25° C. for 2 h and then the reaction mixture was purified by flash C18-flash chromatography, elution gradient 0 to 100% MeCN in water (0.1% NH₄HCO₃) to give the title compound (160 mg, 64.2%) as a pale yellow solid. ¹H NMR δ 0.89 (6H, td), 1.22-1.47 (8H, m), 1.44-1.54 (4H, m), 1.72 (2H, d), 1.82 (2H, s), 1.89 (1H, d), 1.95 (3H, s), 2.82 (4H, d), 3.35-3.41 (1H, m), 3.56 (2H, q), 3.94 (3H, d), 4.19 (1H, d), 6.86 (1H, d), 7.12-7.27 (2H, m), 7.33 (1H, d), 7.70 (1H, dd), 8.32 (1H, d), 8.36-8.45 (1H, m), 12.30 (1H, s). no mass ion

### Intermediate 162d

### N-[1-(4-Chloro-3-cyano-1H-indol-7-yl)piperidin-4-yl]-2-cyano-4-(4-formylpiperidin-1-yl)benzamide

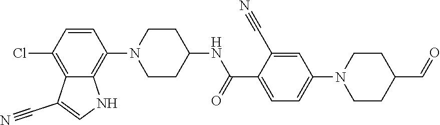 [SMILES: N#Cc1cc(N2CCC(C=O)CC2)ccc1C(=O)NC1CCN(c2ccc(Cl)c3c(C#N)c[nH]c23)CC1]

### N-[1-(4-Chloro-3-cyano-1H-indol-7-yl)piperidin-4-yl]-2-cyano-4-[4-(dibutoxymethyl)piperidin-1-yl]benzamide (210 mg, 0.33 mmol) was stirred in formic acid (3 mL) at 60° C. for 1 h. The reaction was cooled to RT and evaporated to dryness to give the title compound (160 mg, 95%) as a yellow solid which was used without further purification. m/z: ES⁺ [M+H]⁺=515.2. no nmr data

### Example 162

### N-[1-(4-Chloro-3-cyano-1H-indol-7-yl)piperidin-4-yl]-2-cyano-4-[4-({4-[4-(2,4-dioxo-1,3-diazinan-1-yl)-1H-indol-1-yl]piperidin-1-yl}methyl)piperidin-1-yl]benzamide

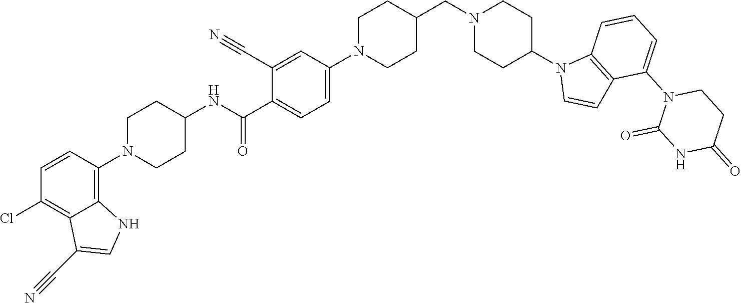 [SMILES: N#Cc1cc(N2CCC(CN3CCC(n4ccc5c(N6CCC(=O)NC6=O)cccc54)CC3)CC2)ccc1C(=O)NC1CCN(c2ccc(Cl)c3c(C#N)c[nH]c23)CC1]

The title compound was prepared using methodology described in example 140 using N-[1-(4-chloro-3-cyano-1H-indol-7-yl)piperidin-4-yl]-2-cyano-4-(4-formylpiperidin-1-yl)benzamide and intermediate 5f to give the title compound in the form of a formate salt (44 mg, 17%) as a white solid. ¹H NMR δ 1.19 (2H, s), 1.77-2.07 (10H, m), 2.13-2.36 (5H, m), 2.7-2.86 (6H, m), 2.84-3.04 (2H, m), 3.37 (2H, d), 3.79 (2H, td), 3.94 (3H, s), 4.39 (1H, s), 6.43 (1H, s), 6.86 (1H, dd), 6.98 (1H, s), 7.1-7.2 (2H, m), 7.25 (1H, d), 7.36 (1H, s), 7.47-7.55 (2H, m), 7.69 (1H, d), 8.32 (1H, d), 8.42 (1H, d), 10.34 (1H, s), 12.31 (1H, s); m/z: ES⁺ [M+H]⁺=811.4.

### Example 163

### Intermediate 163a

### Methyl 4-[4-(dibutoxymethyl)piperidin-1-yl]-2-fluoro-6-methylbenzoate

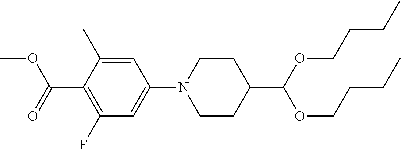 [SMILES: CCCCOC(OCCCC)C1CCN(c2cc(C)c(C(=O)OC)c(F)c2)CC1]

The title compound was prepared using methodology described in intermediate 11a using methyl 4-bromo-2-fluoro-6-methylbenzoate and 4-(dibutoxymethyl)piperidine to give the title compound (2.3 g, 46%) as a white solid. ¹H NMR δ 0.86 (6H, t), 1.2-1.37 (5H, m), 1.39-1.53 (5H, m), 1.72 (3H, dd), 2.31 (3H, s), 2.72 (2H, td), 3.36 (2H, dt), 3.53 (2H, dt), 3.75 (3H, s), 3.85 (2H, d), 4.14 (1H, d), 6.44-6.7 (2H, m); m/z: ES⁺ [M+H]⁺=410.1.

### Intermediate 163b

### 4-[4-(Dibutoxymethyl)piperidin-1-yl]-2-fluoro-6-methylbenzoic acid

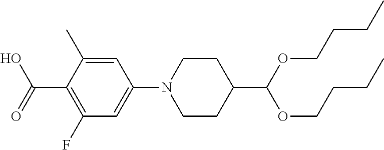 [SMILES: CCCCOC(OCCCC)C1CCN(c2cc(C)c(C(=O)O)c(F)c2)CC1]

The title compound was prepared using methodology described in intermediate 162b using methyl 4-[4-(dibutoxymethyl)piperidin-1-yl]-2-fluoro-6-methylbenzoate to give the title compound (1.6 g, 97%) as a white solid. ¹H NMR δ 0.88 (6H, t), 1.18-1.4 (6H, m), 1.48 (4H, dq), 1.69 (3H, d), 2.29 (3H, s), 2.6-2.73 (2H, m), 3.37 (2H, dt), 3.54 (2H, dt), 3.79 (2H, d), 4.16 (1H, d), 6.43-6.61 (2H, m); m/z: ES⁺ [M+H]⁺=394.2.

### Intermediate 163c

### N-[1-(4-Chloro-3-cyano-1H-indol-7-yl)piperidin-4-yl]-4-[4-(dibutoxymethyl)piperidin-1-yl]-2-fluoro-6-methylbenzamide

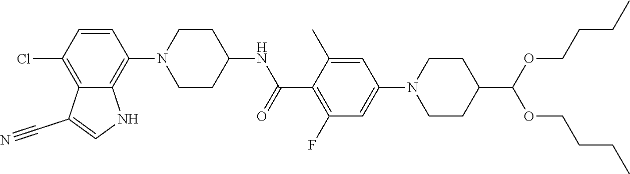 [SMILES: CCCCOC(OCCCC)C1CCN(c2cc(C)c(C(=O)NC3CCN(c4ccc(Cl)c5c(C#N)c[nH]c45)CC3)c(F)c2)CC1]

The title compound was prepared using methodology described in intermediate 152f using 4-[4-(dibutoxymethyl)piperidin-1-yl]-2-fluoro-6-methylbenzoic acid and intermediate 153d to give the title compound (180 mg, 55%) as a yellow gum. m/z: ES⁺ [M+H]⁺=652.4. no nmr data

### Example 163

### N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluoro-6-methylbenzamide

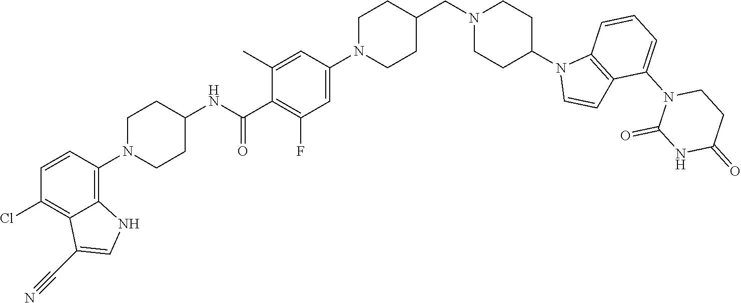 [SMILES: Cc1cc(N2CCC(CN3CCC(n4ccc5c(N6CCC(=O)NC6=O)cccc54)CC3)CC2)cc(F)c1C(=O)NC1CCN(c2ccc(Cl)c3c(C#N)c[nH]c23)CC1]

The title compound was prepared using methodology described in example 140 using N-[1-(4-chloro-3-cyano-1H-indol-7-yl)piperidin-4-yl]-4-[4-(dibutoxymethyl)piperidin-1-yl]-3-fluoro-5-methylbenzamide and intermediate 5f to give the title compound in the form of a formate salt (84 mg, 37%) as a white solid. ¹H NMR δ 1.11-1.26 (2H, m), 1.7-1.89 (5H, m), 1.97 (6H, t), 2.12-2.31 (7H, m), 2.69-2.82 (6H, m), 3.00 (2H, d), 3.39 (2H, s), 3.72-3.83 (4H, m), 3.90 (1H, d), 4.39 (1H, dq), 6.42 (1H, d), 6.53-6.59 (1H, m), 6.63 (1H, d), 6.80 (1H, d), 6.97 (1H, d), 7.06-7.22 (2H, m), 7.47-7.59 (2H, m), 8.27 (1H, s), 8.37 (1H, d), 10.33 (1H, s), 10.35 (1H, s); m/z: ES⁺ [M+H]⁺=818.3.

### Example 164

### Intermediate 164a

### Methyl 4-[4-(dibutoxymethyl)piperidin-1-yl]-3-methylbenzoate

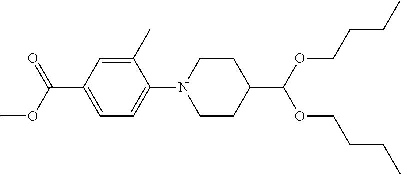 [SMILES: CCCCOC(OCCCC)C1CCN(c2ccc(C(=O)OC)cc2C)CC1]

The title compound was prepared using methodology described in intermediate 11a using methyl 4-bromo-3-methylbenzoate and 4-(dibutoxymethyl)piperidine to give the title compound (1.7 g, 33%) as a yellow gum. ¹H NMR δ 0.90 (6H, t), 1.18 (1H, t), 1.33 (3H, ddd), 1.34-1.43 (2H, m), 1.43-1.58 (4H, m), 1.62-1.81 (2H, m), 1.99 (1H, s), 2.26 (3H, s), 2.59 (2H, t), 3.18 (2H, d), 3.41 (2H, dt), 3.57 (2H, dt), 3.80 (3H, s), 4.23 (1H, d), 7.04 (1H, d), 7.69-7.83 (2H, m); m/z: ES⁺ [M+H]⁺=392.3.

### Intermediate 164b

### 4-[4-(Dibutoxymethyl)piperidin-1-yl]-3-methylbenzoic acid

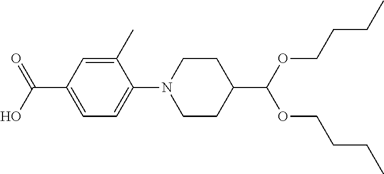 [SMILES: CCCCOC(OCCCC)C1CCN(c2ccc(C(=O)O)cc2C)CC1]

The title compound was prepared using methodology described in intermediate 162b using methyl 4-[4-(dibutoxymethyl)piperidin-1-yl]-3-methylbenzoate to give the title compound (1.35 g, 82%) as a black solid. ¹H NMR δ 0.72 (6H, t), 1.07-1.25 (6H, m), 1.26-1.39 (4H, m), 1.53 (2H, d), 1.61 (1H, s), 2.07 (3H, s), 2.33-2.46 (2H, m), 2.96 (2H, d), 3.23 (2H, dt), 3.39 (2H, dt), 4.05 (1H, d), 6.76-6.85 (1H, m), 7.53 (2H, d) (1H not observed); m/z: ES⁺ [M+H]⁺=378.1.

### Intermediate 164c

### N-[1-(4-Chloro-3-cyano-1H-indol-7-yl)piperidin-4-yl]-4-[4-(dibutoxymethyl)piperidin-1-yl]-3-methylbenzamide

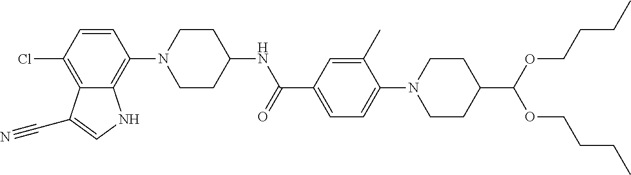 [SMILES: CCCCOC(OCCCC)C1CCN(c2ccc(C(=O)NC3CCN(c4ccc(Cl)c5c(C#N)c[nH]c45)CC3)cc2C)CC1]

The title compound was prepared using methodology described in intermediate 153e using 4-[4-(dibutoxymethyl)piperidin-1-yl]-3-methylbenzoic acid and intermediate 153d to give the title compound (220 mg, 65%) as a yellow gum. ¹H NMR δ 0.90 (5H, t), 1.12-1.7 (8H, m), 1.73-2 (2H, m), 2.28 (3H, s), 2.70 (9H, s), 2.92 (OH, s), 3.09-3.17 (2H, m), 3.32 (5H, s), 3.36-3.45 (4H, m), 3.47-3.63 (1H, m), 4.25 (1H, d), 6.86 (1H, d), 7.02 (1H, d), 7.16 (1H, d), 7.67 (2H, d), 8.19 (1H, d), 8.32 (1H, d), 12.29 (1H, s); m/z: ES⁺ [M+H]⁺=632.3.

### Example 164

### N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-3-methylbenzamide

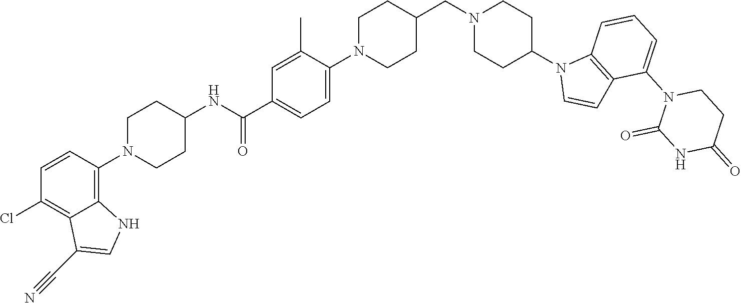 [SMILES: Cc1cc(C(=O)NC2CCN(c3ccc(Cl)c4c(C#N)c[nH]c34)CC2)ccc1N1CCC(CN2CCC(n3ccc4c(N5CCC(=O)NC5=O)cccc43)CC2)CC1]

The title compound was prepared using methodology described in example 140 using N-[1-(4-chloro-3-cyano-1H-indol-7-yl)piperidin-4-yl]-4-[4-(dibutoxymethyl)piperidin-1-yl]-3-methylbenzamide and intermediate 5f to give the title compound in the form of a formate salt (72 mg, 26%) as a white solid. ¹H NMR δ 1.32 (2H, d), 1.70 (1H, s), 1.8-2.1 (10H, m), 2.19 (2H, t), 2.29 (5H, s), 2.64 (2H, t), 2.77 (4H, t), 3.02 (2H, d), 3.14 (2H, d), 3.3-3.43 (2H, m), 3.78 (2H, t), 3.93 (1H, d), 4.39 (1H, s), 6.42 (1H, d), 6.85 (1H, d), 7.00 (2H, dd), 7.15 (2H, t), 7.45-7.57 (2H, m), 7.68 (2H, d), 8.19 (1H, d), 8.32 (1H, s), 10.33 (1H, s), 12.31 (1H, s); m/z: ES⁺ [M+H]⁺ =800.3.

### Example 165

### Intermediate 165a

### Methyl 3-cyano-4-[4-(dibutoxymethyl)piperidin-1-yl]benzoate

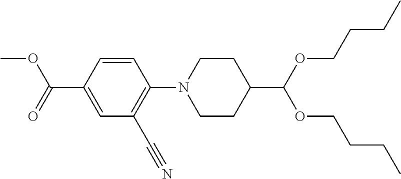 [SMILES: CCCCOC(OCCCC)C1CCN(c2ccc(C(=O)OC)cc2C#N)CC1]

The title compound was prepared using methodology described in intermediate 11a using methyl 4-bromo-3-cyanobenzoate and 4-(dibutoxymethyl)piperidine to give the title compound (2.7 g, 55%) as a pale yellow gum. ¹H NMR δ 0.89 (6H, t), 1.3-1.52 (11H, m), 1.79 (3H, d), 2.85-2.98 (2H, m), 3.40 (2H, dt), 3.57 (2H, dt), 3.80 (4H, s), 4.23 (1H, d), 7.17 (1H, d), 7.95-8.1 (2H, m); m/z: ES⁺ [M+H]⁺=403.1.

### Intermediate 165b

### 3-Cyano-4-[4-(dibutoxymethyl)piperidin-1-yl]benzoic acid

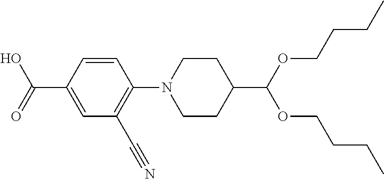 [SMILES: CCCCOC(OCCCC)C1CCN(c2ccc(C(=O)O)cc2C#N)CC1]

The title compound was prepared using methodology described in intermediate 104b using methyl 3-cyano-4-[4-(dibutoxymethyl)piperidin-1-yl]benzoate to give the title compound (2.3 g, 88%) as a white solid. ¹H NMR δ 0.88 (6H, t), 1.28-1.4 (5H, m), 1.42-1.56 (5H, m), 1.77 (3H, d), 2.89 (2H, t), 3.39 (2H, dt), 3.56 (2H, dt), 3.74 (2H, d), 4.23 (1H, d), 7.15 (1H, d), 7.9-8.17 (2H, m), 12.97 (1H, s); m/z: ES⁺ [M+H]⁺=389.1.

### Intermediate 165c

### N-[1-(4-Chloro-3-cyano-1H-indol-7-yl)piperidin-4-yl]-3-cyano-4-[4-(dibutoxymethyl)piperidin-1-yl]benzamide

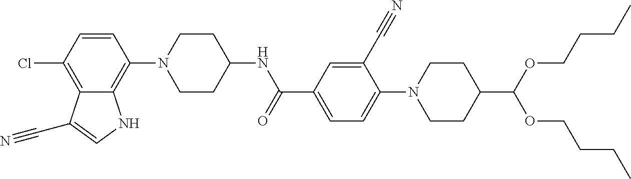 [SMILES: CCCCOC(OCCCC)C1CCN(c2ccc(C(=O)NC3CCN(c4ccc(Cl)c5c(C#N)c[nH]c45)CC3)cc2C#N)CC1]

The title compound was prepared using methodology described in intermediate 153e using 3-cyano-4-[4-(dibutoxymethyl)piperidin-1-yl]benzoic acid and intermediate 153d to give the title compound (200 mg, 60%) as a yellow gum. ¹H NMR δ 0.90 (6H, t), 1.27-1.52 (8H, m), 1.71-1.94 (5H, m), 2.70 (14H, s), 3.52-3.77 (3H, m), 3.94 (1H, s), 4.25 (1H, d), 6.86 (1H, d), 7.17 (1H, dd), 8.05 (1H, d), 8.22 (1H, d), 8.26-8.46 (2H, m), 12.30 (1H, s); m/z: ES⁺ [M+H]⁺=645.2.

### Example 165

### N-[1-(4-Chloro-3-cyano-1H-indol-7-yl)piperidin-4-yl]-3-cyano-4-[4-({4-[4-(2,4-dioxo-1,3-diazinan-1-yl)-1H-indol-1-yl]piperidin-1-yl}methyl)piperidin-1-yl]benzamide

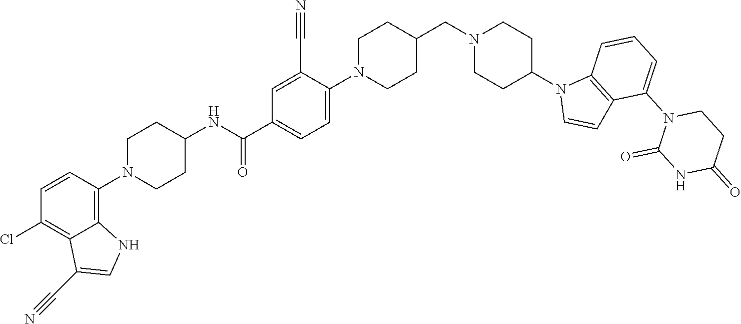 [SMILES: N#Cc1cc(C(=O)NC2CCN(c3ccc(Cl)c4c(C#N)c[nH]c34)CC2)ccc1N1CCC(CN2CCC(n3ccc4c(N5CCC(=O)NC5=O)cccc43)CC2)CC1]

The title compound was prepared using methodology described in example 140 using N-[1-(4-chloro-3-cyano-1H-indol-7-yl)piperidin-4-yl]-3-cyano-4-[4-(dibutoxymethyl)piperidin-1-yl]benzamide and intermediate 5f to give the title compound in the form of a formate salt (69 mg, 30%) as a white solid. ¹H NMR δ 1.33 (2H, t), 1.78 (1H, s), 1.97 (9H, dt), 2.19 (2H, t), 2.29 (2H, d), 2.72-2.86 (4H, m), 2.92 (2H, t), 3.02 (2H, d), 3.40 (2H, d), 3.70 (2H, d), 3.79 (2H, t), 3.94 (1H, s), 4.39 (1H, s), 6.43 (1H, d), 6.84 (1H, d), 6.97 (1H, d), 7.09-7.24 (3H, m), 7.49-7.56 (2H, m), 8.06 (1H, dd), 8.23 (1H, d), 8.32 (1H, s), 8.39 (1H, d), 10.33 (1H, s), 12.34 (1H, s); m/z: ES⁺ [M+H]⁺=811.4.

### Example 166

### Intermediate 166a

### Methyl 5-(1,4-dioxa-8-azaspiro[4.5]decan-8-yl)pyridine-2-carboxylate

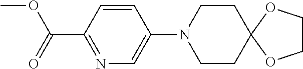 [SMILES: COC(=O)c1ccc(N2CCC3(CC2)OCCO3)cn1]

The title compound was prepared using methodology described in intermediate 63a using methyl 5-fluoropyridine-2-carboxylate and 1,4-dioxa-8-azaspiro[4.5]decane to give the title compound (2.5 g, 69%) as a yellow solid. ¹H NMR δ 1.63-1.73 (4H, m), 3.44-3.56 (4H, m), 3.79 (3H, s), 3.91 (4H, s), 7.35 (1H, dd), 7.84 (1H, d), 8.38 (1H, d); m/z: ES⁺ [M+H]⁺=279.0.

### Intermediate 166b

### 5-(1,4-Dioxa-8-azaspiro[4.5]decan-8-yl)pyridine-2-carboxylic acid

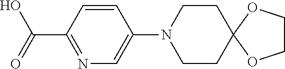 [SMILES: O=C(O)c1ccc(N2CCC3(CC2)OCCO3)cn1]

LiOH (2.5 g, 106.25 mmol) was added to methyl 5-(1,4-dioxa-8-azaspiro[4.5]decan-8-yl)pyridine-2-carboxylate (2.5 g, 10.63 mmol) in THF:water (1:1, 50 mL) at RT and stirred for 1 h. The solvent was removed under reduced pressure to afford and the aqueous was neutralised to pH7 using HCl (2M). The solid was filtered under vacuum to give the title compound (3.00 g, 107%) which was used without further purification. ¹H NMR δ 1.65 (4H, t), 3.35-3.42 (4H, m), 3.88 (4H, s), 7.43 (1H, dd), 7.83 (1H, d), 8.11 (1H, d) 1H not observed; m/z: ES⁺ [M+H]⁺=265.0.

### Intermediate 166c

### N-[1-(4-Chloro-3-cyano-1H-indol-7-yl)piperidin-4-yl]-5-(1,4-dioxa-8-azaspiro[4.5]decan-8-yl)pyridine-2-carboxamide

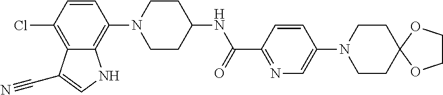 [SMILES: N#Cc1c[nH]c2c(N3CCC(NC(=O)c4ccc(N5CCC6(CC5)OCCO6)cn4)CC3)ccc(Cl)c12]

Propane phosphonic acid anhydride (50%, 463 mg, 0.73 mmol) was added to 5-(1,4-dioxa-8-azaspiro[4.5]decan-8-yl)pyridine-2-carboxylic acid (144 mg, 0.55 mmol), intermediate 153d (100 mg, 0.36 mmol) and triethylamine (0.152 mL, 1.09 mmol) in THF (2 mL) and stirred at 40° C. for 2 h. The solvent was removed under reduced pressure afford crude product. The crude product was purified by flash silica chromatography, elution gradient 0 to 100% EtOAc in Et₂O to give the title compound (60.0 mg, 31.6%) as a brown solid. ¹H NMR δ 1.71 (8H, t), 1.9-1.98 (5H, m), 3.44-3.54 (4H, m), 4.23 (1H, t), 6.86 (1H, d), 7.16 (1H, d), 7.34-7.5 (2H, m), 7.86 (2H, d), 8.27-8.42 (4H, m), 12.32 (1H, s); m/z: ES⁺ [M+H]⁺=521.0.

### Example 166

### N-[1-(4-Chloro-3-cyano-1H-indol-7-yl)piperidin-4-yl]-5-(4-{4-[2-(2,6-dioxopiperidin-3-yl)-6-fluoro-1,3-dioxo-2,3-dihydro-1H-isoindol-5-yl]piperazin-1-yl}piperidin-1-yl)pyridine-2-carboxamide

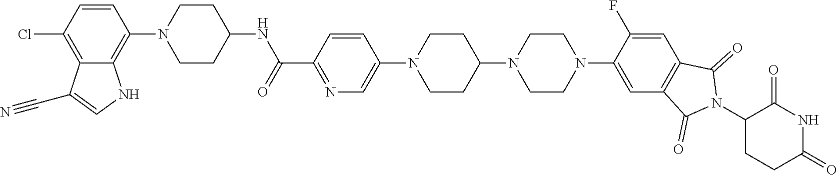 [SMILES: N#Cc1c[nH]c2c(N3CCC(NC(=O)c4ccc(N5CCC(N6CCN(c7cc8c(cc7F)C(=O)N(C7CCC(=O)NC7=O)C8=O)CC6)CC5)cn4)CC3)ccc(Cl)c12]

N-[1-(4-Chloro-3-cyano-1H-indol-7-yl)piperidin-4-yl]-5-(1,4-dioxa-8-azaspiro[4.5]decan-8-yl)pyridine-2-carboxamide (50 mg, 0.10 mmol) was added to formic acid (1 mL) and stirred at 40° C. for 1 h. The solvent was evaporated to give a residue. Intermediate 50b (53.0 mg, 0.12 mmol) was also added to formic acid (1 mL) and attired at RT for 1 h. The solvent was evaporated to give a residue. The two residues were combined in NMP (2 mL) and sodium triacetoxyborohydride (40.7 mg, 0.19 mmol) was added. The mixture was stirred at RT for 16 h. The reaction mixture was purified by flash C18-flash chromatography, elution gradient 5 to 70% MeCN in water (0.1% NH₄HCO₃) to give the title compound (18 mg, 22.8%) as a yellow solid. ¹H NMR δ 1.54 (3H, d), 1.86-2.09 (7H, m), 2.61 (3H, s), 2.69 (4H, s), 2.76-2.97 (5H, m), 3.25 (4H, s), 3.98 (3H, d), 5.11 (1H, dd), 6.86 (1H, d), 7.16 (1H, d), 7.4-7.49 (2H, m), 7.73 (1H, d), 7.86 (1H, d), 8.27 (1H, d), 8.32 (2H, s), 11.11 (1H, s), 12.33 (1H, s); m/z: ES⁺ [M+H]⁺=821.3.

### Example 167

### N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyridine-2-carboxamide

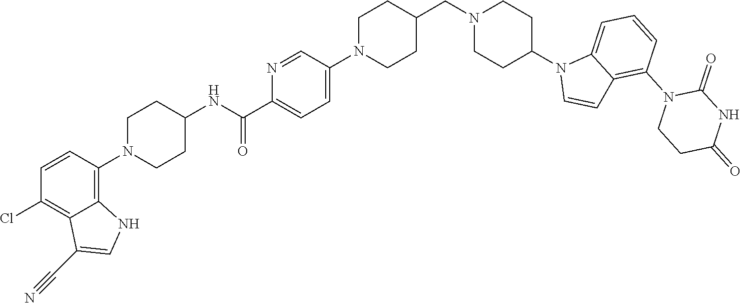 [SMILES: N#Cc1c[nH]c2c(N3CCC(NC(=O)c4ccc(N5CCC(CN6CCC(n7ccc8c(N9CCC(=O)NC9=O)cccc87)CC6)CC5)cn4)CC3)ccc(Cl)c12]

The title compound was prepared using methodology described in example 62 using intermediate 166c and intermediate 5f to give the title compound (16 mg, 8%) as a white solid. ¹H NMR δ 1.24 (3H, s), 1.90 (11H, d), 2.22 (4H, d), 2.69-2.92 (7H, m), 3.01 (2H, d), 3.78 (2H, t), 3.94 (3H, d), 4.39 (1H, s), 6.43 (1H, d), 6.87 (1H, d), 6.97 (1H, d), 7.15 (2H, t), 7.38-7.47 (1H, m), 7.47-7.59 (2H, m), 7.86 (1H, d), 8.25-8.36 (3H, m), 10.34 (1H, s), 12.32 (1H, s); m/z: ES⁺ [M+H]⁺=787.4.

### Example 168

### N-[1-(4-Chloro-3-cyano-1H-indol-7-yl)piperidin-4-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide

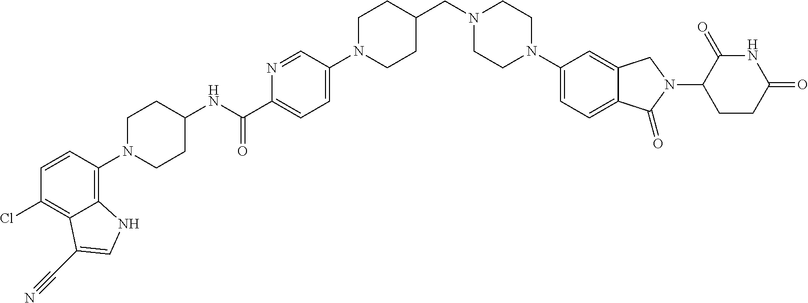 [SMILES: N#Cc1c[nH]c2c(N3CCC(NC(=O)c4ccc(N5CCC(CN6CCN(c7ccc8c(c7)CN(C7CCC(=O)NC7=O)C8=O)CC6)CC5)cn4)CC3)ccc(Cl)c12]

The title compound was prepared using methodology described in example 24 using intermediate 166c and intermediate 1i to give the title compound (62 mg, 17%) as a white solid. ¹H NMR δ 1.21 (3H, d), 1.77-2.05 (9H, m), 2.23 (2H, d), 2.22-2.43 (1H, m), 2.62 (4H, s), 2.78-2.98 (6H, m), 3.34 (4H, s), 3.93 (3H, d), 4.1-4.49 (2H, m), 5.06 (1H, dd), 6.86 (1H, d), 7.07 (2H, d), 7.16 (1H, d), 7.36-7.46 (1H, m), 7.53 (1H, d), 7.85 (1H, d), 8.25-8.36 (3H, m), 10.96 (1H, s), 12.32 (1H, s); m/z: ES⁺ [M+H]⁺=803.3.

### Example 169

### N-[1-(4-Chloro-3-cyano-1H-indol-7-yl)piperidin-4-yl]-5-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]pyridine-2-carboxamide

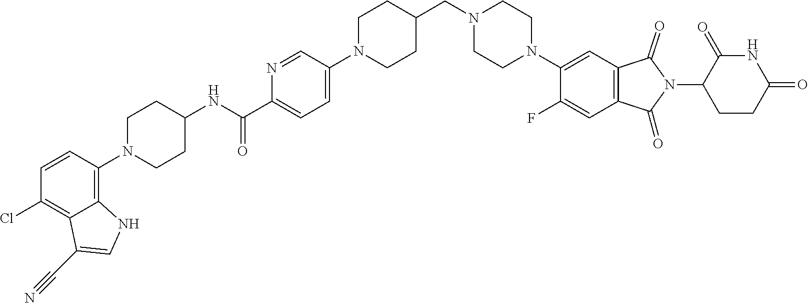 [SMILES: N#Cc1c[nH]c2c(N3CCC(NC(=O)c4ccc(N5CCC(CN6CCN(c7cc8c(cc7F)C(=O)N(C7CCC(=O)NC7=O)C8=O)CC6)CC5)cn4)CC3)ccc(Cl)c12]

The title compound was prepared using methodology described in example 24 using intermediate 166c and intermediate 50b to give the title compound (45 mg, 15%) as a white solid. ¹H NMR δ 1.22 (3H, d), 1.84 (3H, d), 1.95 (5H, s), 2.24 (2H, d), 2.55 (6H, s), 2.87 (6H, t), 3.27 (4H, s), 3.93 (3H, d), 5.11 (1H, dd), 6.86 (1H, d), 7.16 (1H, d), 7.36-7.51 (2H, m), 7.74 (1H, d), 7.85 (1H, d), 8.30 (3H, t), 11.12 (1H, s), 12.31 (1H, s); m/z: ES⁺ [M+H]⁺=835.4.

### Example 170

### Intermediate 170a

### Methyl 5-[4-(dibutoxymethyl)piperidin-1-yl]pyridine-2-carboxylate

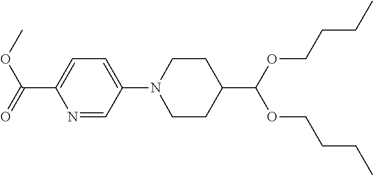 [SMILES: CCCCOC(OCCCC)C1CCN(c2ccc(C(=O)OC)nc2)CC1]

The title compound was prepared using methodology described in intermediate 63a using methyl 5-fluoropyridine-2-carboxylate and 4-(dibutoxymethyl)piperidine to give the title compound (3.5 g, 71%) as a white solid. ¹H NMR δ 0.88 (6H, t), 1.25 (1H, dd), 1.26-1.4 (5H, m), 1.42-1.53 (4H, m), 1.69-1.77 (2H, m), 1.77-1.84 (1H, m), 2.83 (2H, t), 3.34-3.43 (2H, m), 3.5-3.6 (2H, m), 3.79 (3H, s), 3.98 (2H, d), 4.18 (1H, d), 7.31 (1H, dd), 7.84 (1H, d), 8.35 (1H, d); m/z: ES⁺ [M+H]⁺=379.2.

### Intermediate 170b

### 5-[4-(Dibutoxymethyl)piperidin-1-yl]pyridine-2-carboxylic acid

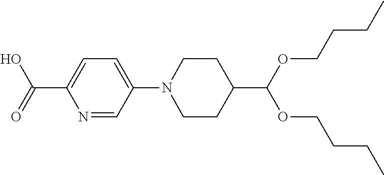 [SMILES: CCCCOC(OCCCC)C1CCN(c2ccc(C(=O)O)nc2)CC1]

The title compound was prepared using methodology described in intermediate 104b using methyl 5-[4-(dibutoxymethyl)piperidin-1-yl]pyridine-2-carboxylate to give the title compound (3.2 g, 96%) as a yellow solid. ¹H NMR δ 0.88 (6H, t), 1.21-1.3 (1H, m), 1.26-1.4 (5H, m), 1.48 (4H, dq), 1.73 (2H, d), 1.77-1.84 (1H, m), 2.77-2.88 (2H, m), 3.33-3.43 (2H, m), 3.5-3.6 (2H, m), 3.98 (2H, d), 4.18 (1H, d), 7.32 (1H, dd), 7.83 (1H, d), 8.33 (1H, d), 12.38 (1H, s); m/z: ES⁺ [M+H]⁺=365.2.

### Intermediate 170c

### N-[1-(4-Chloro-3-cyano-1H-indazol-7-yl)piperidin-4-yl]-5-[4-(dibutoxymethyl)piperidin-1-yl]pyridine-2-carboxamide

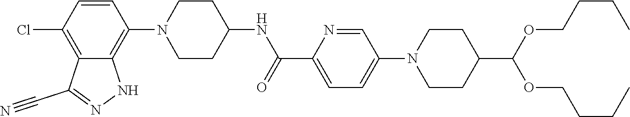 [SMILES: CCCCOC(OCCCC)C1CCN(c2ccc(C(=O)NC3CCN(c4ccc(Cl)c5c(C#N)n[nH]c45)CC3)nc2)CC1]

The title compound was prepared using methodology described in intermediate 152f using 5-[4-(dibutoxymethyl)piperidin-1-yl]pyridine-2-carboxylic acid and intermediate 152e to give the title compound (500 mg, 97%) as a white solid. ¹H NMR δ 0.89 (6H, t), 1.2-1.41 (6H, m), 1.37-1.57 (4H, m), 1.75 (2H, d), 1.95 (4H, s), 2.69 (4H, s), 2.84 (3H, s), 3.35-3.46 (2H, m), 3.57 (2H, dt), 3.95 (3H, d), 4.20 (1H, d), 6.94 (1H, dd), 7.01-7.13 (1H, m), 7.40 (1H, dd), 7.85 (1H, d), 8.26-8.35 (2H, m), 14.73 (1H, s); m/z: ES⁻[M−H]⁻=620.3.

### Example 170

### N-[1-(4-Chloro-3-cyano-1H-indazol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyridine-2-carboxamide

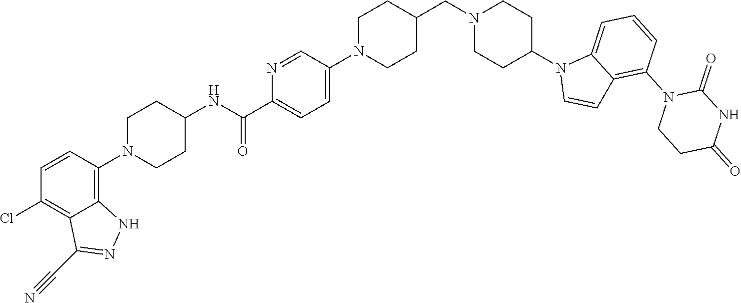 [SMILES: N#Cc1n[nH]c2c(N3CCC(NC(=O)c4ccc(N5CCC(CN6CCC(n7ccc8c(N9CCC(=O)NC9=O)cccc87)CC6)CC5)cn4)CC3)ccc(Cl)c12]

The title compound was prepared using methodology described in example 62 using N-[1-(4-chloro-3-cyano-1H-indazol-7-yl)piperidin-4-yl]-5-[4-(dibutoxymethyl)piperidin-1-yl]pyridine-2-carboxamide and intermediate 5f to give the title compound (5 mg, 4%) as a white solid. ¹H NMR δ 1.22 (3H, d), 1.89 (11H, d), 2.12-2.35 (4H, m), 2.77 (2H, t), 2.91 (4H, s), 3.01 (2H, d), 3.63 (2H, s), 3.78 (2H, t), 3.94 (3H, d), 4.39 (1H, s), 6.42 (1H, d), 6.82 (1H, d), 6.97 (1H, d), 7.05-7.23 (2H, m), 7.42 (1H, dd), 7.47-7.59 (2H, m), 7.86 (1H, d), 8.27-8.37 (2H, m), 10.35 (1H, s); m/z: ES⁺ [M+H]⁺=788.4.

### Example 171

### Intermediate 171a

### 7-(1,4-Dioxa-8-azaspiro[4.5]decan-8-yl)-4-methyl-1H-indole-3-carbonitrile

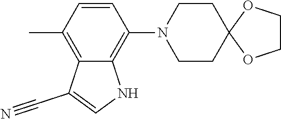 [SMILES: Cc1ccc(N2CCC3(CC2)OCCO3)c2[nH]cc(C#N)c12]

The title compound was prepared using methodology described in intermediate 57b using intermediate 8a and 1,4-dioxa-8-azaspiro[4.5]decane to give the title compound (729 mg, 77%) as a white solid. ¹H NMR δ 1.89-1.99 (4H, m), 2.64 (3H, s), 3.02-3.13 (4H, m), 3.98 (4H, s), 6.81 (1H, d), 6.91 (1H, dd), 8.22 (1H, d), 11.96 (1H, s); m/z: ES⁺ [M+H]⁺=298.3.

### Intermediate 171b

### 7-(4-{[(4-Methoxyphenyl)methyl]amino}piperidin-1-yl)-4-methyl-1H-indole-3-carbonitrile

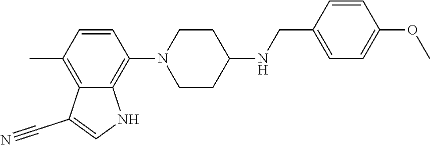 [SMILES: COc1ccc(CNC2CCN(c3ccc(C)c4c(C#N)c[nH]c34)CC2)cc1]

7-(1,4-Dioxa-8-azaspiro[4.5]decan-8-yl)-4-methyl-1H-indole-3-carbonitrile (791 mg, 2.66 mmol) was stirred in formic acid (18 mL, 497.44 mmol) at 60° C. for 4 h. The reaction mixture was evaporated and the residue was suspended in NMP (2 mL) at RT. 4-Methoxybenzylamine (0.695 mL, 5.32 mmol) was added and the mixture was stirred for 5 mins followed by the addition of sodium triacetoxyhydroborate (1.9 g, 9.31 mmol) and was stirred at RT for 2 h. The reaction mixture was diluted with EtOAc (75 mL), washed with water (2×75 mL), saturated brine (50 mL), dried over a phase separating cartridge and the solvent was evaporated to afford crude product. The crude product was purified by flash silica chromatography, elution gradient 0 to 100% EtOAc in heptane followed by MeOH (10%) in EtOAc to give the title compound (0.743 g, 74.6%) as a yellow solid. ¹H NMR δ 1.9-2.01 (6H, m), 2.23 (4H, t), 2.64 (3H, s), 2.93-3.04 (1H, m), 3.82 (3H, s), 6.78 (1H, d), 6.86-6.96 (1H, m), 6.98-7.08 (2H, m), 7.51 (2H, d), 8.11-8.38 (2H, m), 12.04 (1H, s); m/z: ES⁺ [M+H]⁺=375.1.

### Intermediate 171c

### 7-(4-Aminopiperidin-1-yl)-4-methyl-1H-indole-3-carbonitrile

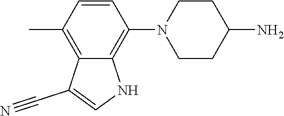 [SMILES: Cc1ccc(N2CCC(N)CC2)c2[nH]cc(C#N)c12]

7-(4-{[(4-Methoxyphenyl)methyl]amino}piperidin-1-yl)-4-methyl-1H-indole-3-carbonitrile (743 mg, 1.98 mmol) was dissolved in ethanol (40 mL), acetic acid (200 μL) and Pd(OH)₂/C (20%, 139 mg, 0.20 mmol) was added. The mixture was hydrogenated at atmospheric pressure and RT for 48 h. The mixture was filtered through Celite® and the solvent was evaporated to give the title compound (0.193 g, 38.2%) as a beige solid. ¹H NMR δ 1.53-1.68 (2H, m), 1.88 (2H, d), 2.61-2.65 (3H, m), 2.66-2.75 (2H, m), 3.23-3.44 (3H, m), 6.78 (1H, d), 6.90 (1H, dd), 8.18 (1H, s) 3 exchangeable H not observed; m/z: ES⁺ [M+H]⁺=255.2.

### Intermediate 171d

### Methyl 5-[4-(1,3-dioxolan-2-yl)piperidin-1-yl]pyridine-2-carboxylate

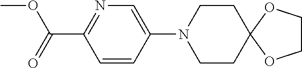 [SMILES: COC(=O)c1ccc(N2CCC3(CC2)OCCO3)cn1]

The title compound was prepared using methodology described in intermediate 63a using methyl 5-fluoropyridine-2-carboxylate and 4-(1,3-dioxolan-2-yl)piperidine to give the title compound (960 mg, 55%) as a white solid. ¹H NMR (CDCl₃) δ 1.55 (2H, s), 1.82 (1H, tq), 1.88 (2H, d), 2.90 (2H, td), 3.85-4.01 (9H, m), 4.69 (1H, d), 7.14 (1H, dd), 7.94-8.02 (1H, m), 8.34 (1H, d); m/z: ES⁺ [M+H]⁺=293.2.

### Intermediate 171e

### 5-[4-(1,3-Dioxolan-2-yl)piperidin-1-yl]pyridine-2-carboxylic acid

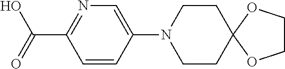 [SMILES: O=C(O)c1ccc(N2CCC3(CC2)OCCO3)cn1]

LiOH (0.617 g, 14.71 mmol) in water (10 mL) was added to a solution of methyl 5-[4-(1,3-dioxolan-2-yl)piperidin-1-yl]pyridine-2-carboxylate (0.860 g, 2.94 mmol) in THF (10 mL) and MeOH (20 mL). The mixture was stirred at 50° C. for 30 mins and then cooled to RT. The solvent was evaporated and the residue was suspended in water (5 mL) and neutralised with citric acid (1M). The product was extracted with DCM (10 mL) and the solvent was evaporated to give the title compound (0.693 g, 85%) as a white solid. ¹H NMR δ 1.37 (2H, qd), 1.66-1.96 (3H, m), 2.87 (2H, td), 3.72-3.94 (4H, m), 4.01 (2H, d), 4.61 (1H, d), 7.34 (1H, dd), 7.84 (1H, d), 8.34 (1H, d); m/z: ES⁺ [M+H]⁺=279.2.

### Intermediate 171f

### N-[1-(3-Cyano-4-methyl-1H-indol-7-yl)piperidin-4-yl]-5-[4-(1,3-dioxolan-2-yl)piperidin-1-yl]pyridine-2-carboxamide

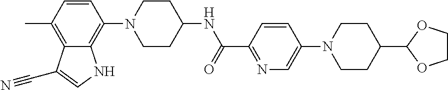 [SMILES: Cc1ccc(N2CCC(NC(=O)c3ccc(N4CCC(C5OCCO5)CC4)cn3)CC2)c2[nH]cc(C#N)c12]

The title compound was prepared using methodology described in intermediate 152f using 7-(4-aminopiperidin-1-yl)-4-methyl-1H-indole-3-carbonitrile and 5-[4-(1,3-dioxolan-2-yl)piperidin-1-yl]pyridine-2-carboxylic acid to give the title compound (729 mg, 77%) as a white solid. ¹H NMR δ 1.38-1.51 (2H, m), 1.73-1.86 (4H, m), 1.96-2.07 (4H, m), 2.65 (3H, s), 2.82-2.94 (4H, m), 3.34 (1H, s), 3.78-3.89 (3H, m), 3.89-3.97 (3H, m), 4.04 (2H, s), 6.83 (1H, d), 6.93 (1H, dd), 7.44-7.51 (1H, m), 7.90 (1H, d), 8.22 (1H, s), 8.30 (1H, d), 8.35 (1H, s), 11.98 (1H, s); m/z: ES⁺ [M+H]⁺=515.5.

### Example 171

### N-[1-(3-Cyano-4-methyl-1H-indol-7-yl)piperidin-4-yl]-5-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]pyridine-2-carboxamide

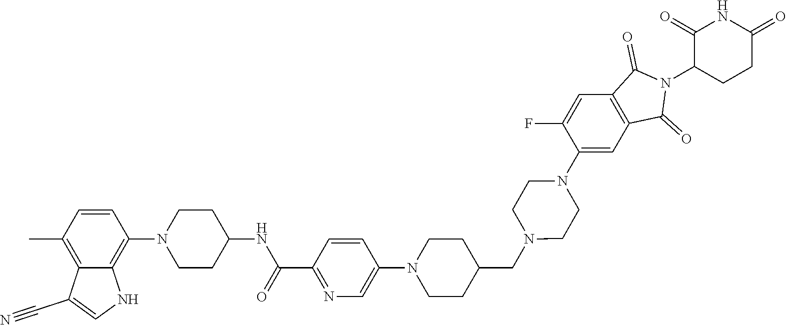 [SMILES: Cc1ccc(N2CCC(NC(=O)c3ccc(N4CCC(CN5CCN(c6cc7c(cc6F)C(=O)N(C6CCC(=O)NC6=O)C7=O)CC5)CC4)cn3)CC2)c2[nH]cc(C#N)c12]

N-[1-(3-Cyano-4-methyl-1H-indol-7-yl)piperidin-4-yl]-5-[4-(1,3-dioxolan-2-yl)piperidin-1-yl]pyridine-2-carboxamide (88 mg, 0.17 mmol) and intermediate 50b (87 mg, 0.19 mmol) were heated in formic acid (1 mL, 26.51 mmol) at 60° C. for 2 h and then cooled to RT. The mixture was concentrated and the residue was suspended in NMP (2 mL) at RT. The reaction mixture was stirred for 5 mins followed by addition of sodium triacetoxyhydroborate (91 mg, 0.43 mmol). The suspension was stirred at RT for 2 h. The reaction mixture was purified by flash C18-flash chromatography, elution gradient 5 to 70% MeCN in water (0.1% formic acid) to give the title compound in the form of a formate salt (5.5 mg, 37.4%) as a yellow solid. ¹H NMR δ 1.23 (3H, d), 1.84 (3H, d), 1.95 (4H, d), 2.04 (1H, dd), 2.25 (2H, d), 2.55-2.66 (7H, m), 2.72-2.98 (5H, m), 3.27 (4H, s), 3.93 (3H, d), 5.11 (1H, dd), 6.78 (1H, d), 6.88 (1H, d), 7.36-7.52 (2H, m), 7.74 (1H, d), 7.85 (1H, d), 8.09-8.2 (1H, m), 8.24 (1H, d), 8.30 (1H, d), 11.10 (1H, s) 4H under DMSO; m/z: ES⁺ [M+H]⁺=815.6.

### Example 172

### N-[1-(3-Cyano-4-methyl-1H-indol-7-yl)piperidin-4-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide

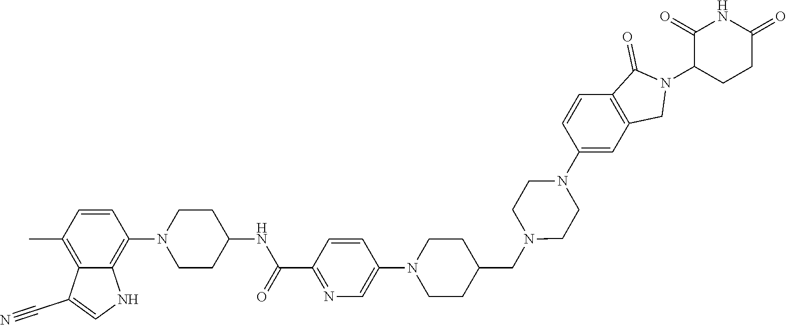 [SMILES: Cc1ccc(N2CCC(NC(=O)c3ccc(N4CCC(CN5CCN(c6ccc7c(c6)CN(C6CCC(=O)NC6=O)C7=O)CC5)CC4)cn3)CC2)c2[nH]cc(C#N)c12]

The title compound was prepared using methodology described in example 171 using intermediate 171f and intermediate 1i to give the title compound in the form of a formate salt (47 mg, 37%) as a white solid. ¹H NMR δ 1.19-1.38 (3H, m), 1.83-1.95 (3H, m), 1.95-2.11 (5H, m), 2.2-2.33 (2H, m), 2.4-2.47 (1H, m), 2.61-2.71 (4H, m), 2.79-3.03 (5H, m), 3.99 (3H, d), 4.27 (1H, d), 4.39 (1H, d), 5.11 (1H, dd), 6.83 (1H, d), 6.93 (1H, d), 7.13 (2H, d), 7.47 (1H, dd), 7.59 (1H, d), 7.91 (1H, d), 8.17-8.26 (1H, m), 8.30 (1H, d), 8.36 (1H, d), 10.99 (1H, s) 10H under water and DMSO; m/z: ES⁺ [M+H]⁺=783.8.

### Example 173

### Intermediate 173a

### Methyl 4-[4-(dibutoxymethyl)piperidin-1-yl]benzoate

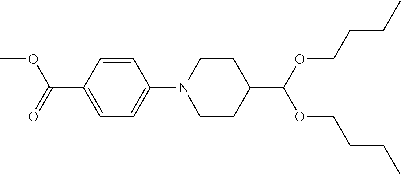 [SMILES: CCCCOC(OCCCC)C1CCN(c2ccc(C(=O)OC)cc2)CC1]

The title compound was prepared using methodology described in intermediate 63a using methyl 4-fluorobenzoate and 4-(dibutoxymethyl)piperidine to give the title compound (2.0 g, 20%) as a white solid. ¹H NMR δ 0.88 (6H, t), 1.18-1.59 (12H, m), 1.75 (3H, t), 2.78 (2H, td), 3.3-3.46 (3H, m), 3.55 (2H, dt), 3.92 (2H, d), 4.17 (1H, d), 6.93-6.97 (2H, m), 7.74-7.8 (2H, m); m/z: ES⁺ [M+H]⁺=378.3.

### Intermediate 173b

### 4-[4-(Dibutoxymethyl)piperidin-1-yl]benzoic acid

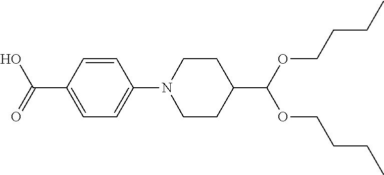 [SMILES: CCCCOC(OCCCC)C1CCN(c2ccc(C(=O)O)cc2)CC1]

The title compound was prepared using methodology described in intermediate 104b using methyl 4-[4-(dibutoxymethyl)piperidin-1-yl]benzoate to give the title compound (1.6 g, 83%) as a white solid. ¹H NMR δ 0.88 (6H, t), 1.15-1.44 (6H, m), 1.4-1.54 (4H, m), 1.75 (3H, t), 2.77 (2H, dd), 3.39 (3H, dt), 3.56 (2H, dt), 3.91 (2H, d), 4.18 (1H, d), 6.86-6.98 (2H, m), 7.69-7.8 (2H, m); m/z: ES⁺ [M+H]⁺=364.2.

### Intermediate 173c

### N-[1-(4-Chloro-3-cyano-1H-indol-7-yl)piperidin-4-yl]-4-[4-(dibutoxymethyl)piperidin-1-yl]benzamide

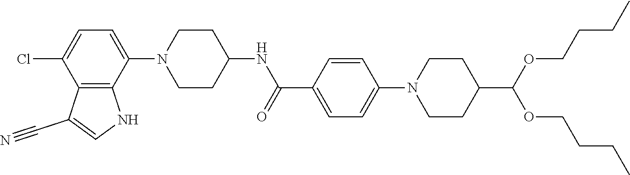 [SMILES: CCCCOC(OCCCC)C1CCN(c2ccc(C(=O)NC3CCN(c4ccc(Cl)c5c(C#N)c[nH]c45)CC3)cc2)CC1]

The title compound was prepared using methodology described in intermediate 152f using 4-[4-(dibutoxymethyl)piperidin-1-yl]benzoic acid and intermediate 153d to give the title compound (170 mg, 66%) as a yellow gum. ¹H NMR δ 0.88 (6H, d), 1.22-1.41 (7H, m), 1.49 (4H, dd), 1.73 (3H, d), 1.93 (4H, d), 2.51 (3H, p), 3.31-3.43 (5H, m), 3.56 (2H, dt), 3.88 (2H, t), 4.18 (1H, d), 6.85 (1H, d), 6.94 (2H, d), 7.16 (1H, d), 7.77 (2H, d), 8.08 (1H, d), 8.32 (1H, d), 12.22-12.34 (1H, m); m/z: ES⁺ [M+H]⁺=620.1.

### Example 173

### N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]benzamide

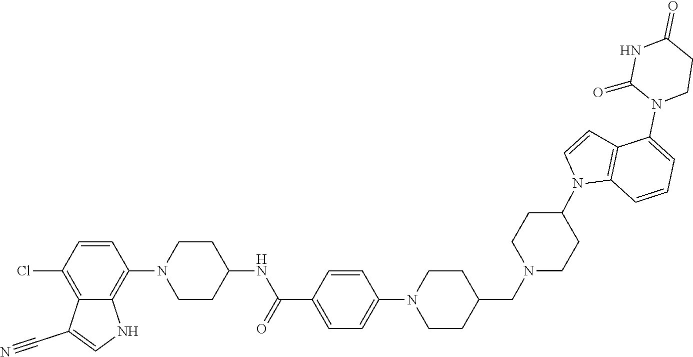 [SMILES: N#Cc1c[nH]c2c(N3CCC(NC(=O)c4ccc(N5CCC(CN6CCC(n7ccc8c(N9CCC(=O)NC9=O)cccc87)CC6)CC5)cc4)CC3)ccc(Cl)c12]

The title compound was prepared using methodology described in example 142 using N-[1-(4-chloro-3-cyano-1H-indol-7-yl)piperidin-4-yl]-4-[4-(dibutoxymethyl)piperidin-1-yl]benzamide and intermediate 5f to give the title compound (39 mg, 18%) as a white solid. ¹H NMR δ 1.20 (2H, d), 1.76-2.07 (11H, m), 2.12-2.29 (4H, m), 2.77 (6H, q), 3.00 (2H, d), 3.45 (2H, d), 3.78 (2H, t), 3.86 (2H, d), 3.93 (1H, d), 4.32-4.42 (1H, m), 6.42 (1H, d), 6.80 (1H, d), 6.96 (3H, dd), 7.09-7.19 (2H, m), 7.48-7.58 (2H, m), 7.77 (2H, d), 8.07 (1H, d), 8.27 (1H, s), 10.33 (1H, s), 12.29 (1H, s); m/z: ES⁺ [M+H]⁺=786.4.

### Example 174

### Intermediate 174a

### 4-Chloro-7-(1,4-dioxaspiro[4.5]dec-7-en-8-yl)-1H-indole-3-carbonitrile

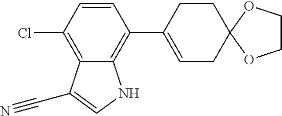 [SMILES: N#Cc1c[nH]c2c(C3=CCC4(CC3)OCCO4)ccc(Cl)c12]

The title compound was prepared using methodology described in intermediate 69a using intermediate 1b and 8-(4,4,5,5-tetramethyl-1,3,2-dioxaborolan-2-yl)-1,4-dioxaspiro[4.5]dec-7-ene to give the title compound (6 g, 97%) as a yellow solid. ¹H NMR δ 1.87 (2H, q), 2.36-2.48 (2H, m), 2.5-2.6 (2H, m), 3.95 (4H, s), 5.89 (1H, td), 7.09 (1H, d), 7.21 (1H, d), 8.35 (1H, s), 12.22 (1H, s); m/z: ES⁺ [M+H]⁺=315.0.

### Intermediate 174b

### 4-Chloro-7-(1,4-dioxaspiro[4.5]decan-8-yl)-1H-indole-3-carbonitrile

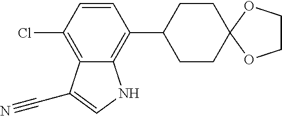 [SMILES: N#Cc1c[nH]c2c(C3CCC4(CC3)OCCO4)ccc(Cl)c12]

The title compound was prepared using methodology described in intermediate 60c using 4-chloro-7-(1,4-dioxaspiro[4.5]dec-7-en-8-yl)-1H-indole-3-carbonitrile to give the title compound (5.5 g, 91%) as a white solid. ¹H NMR δ 1.76 (8H, dd), 1.84-1.91 (1H, m), 3.15 (3H, d), 3.95 (1H, s), 7.15 (2H, dt), 8.38 (1H, d), 12.30 (1H, s); m/z: ES⁺ [M+H]⁺=317.0.

### Intermediate 174c

### 4-Chloro-7-(4-oxocyclohexyl)-1H-indole-3-carbonitrile

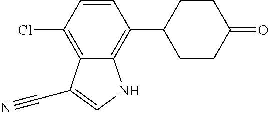 [SMILES: N#Cc1c[nH]c2c(C3CCC(=O)CC3)ccc(Cl)c12]

4-Chloro-7-(1,4-dioxaspiro[4.5]decan-8-yl)-1H-indole-3-carbonitrile (5.5 g, 17.36 mmol) was stirred in formic acid (40 mL) at 45° C. for 16 h. The reaction was cooled to RT and evaporated to dryness to give the title compound (5.0 g, 106%) as a brown solid and was used without further purification. ¹H NMR δ 1.82-1.88 (6H, m), 2.13 (1H, s), 2.51 (3H, p), 6.81 (1H, s), 7.07-7.24 (1H, m), 8.42 (1H, s); m/z: ES⁺ [M+H]⁺=273.0.

### Intermediate 174d

### 7-(4-Aminocyclohexyl)-4-chloro-1H-indole-3-carbonitrile

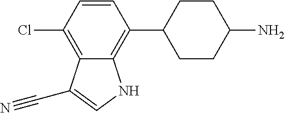 [SMILES: N#Cc1c[nH]c2c(C3CCC(N)CC3)ccc(Cl)c12]

The title compound was prepared using methodology described in intermediate 152e using 4-chloro-7-(4-oxocyclohexyl)-1H-indole-3-carbonitrile to give the title compound (3.0 g, 66%) as a white solid. ¹H NMR δ 1.61 (4H, q), 1.74-2.01 (4H, m), 2.07 (2H, s), 3.09 (2H, s), 7.03-7.22 (2H, m), 8.39 (1H, s) 1H not observed; m/z: ES⁺ [M+H]⁺=274.0.

### Intermediate 174e

### N-[4-(4-Chloro-3-cyano-1H-indol-7-yl)cyclohexyl]-4-[4-(dibutoxymethyl)piperidin-1-yl]-2-fluorobenzamide

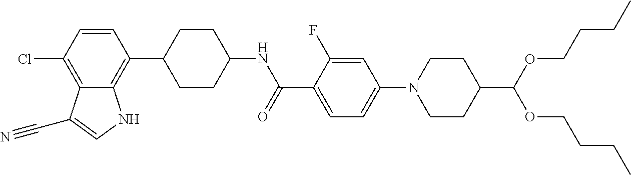 [SMILES: CCCCOC(OCCCC)C1CCN(c2ccc(C(=O)NC3CCC(c4ccc(Cl)c5c(C#N)c[nH]c45)CC3)c(F)c2)CC1]

The title compound was prepared using methodology described in intermediate 152f using 7-(4-aminocyclohexyl)-4-chloro-1H-indole-3-carbonitrile and intermediate 152b to give the title compound (260 mg, 22%) as a white solid. ¹H NMR δ 0.88 (6H, t), 1.13-1.78 (21H, m), 3.04 (1H, s), 3.26-3.42 (2H, m), 3.5-3.58 (2H, m), 3.75-4.03 (5H, m), 4.17 (1H, d), 6.65-6.81 (2H, m), 7.1-7.27 (2H, m), 7.51 (1H, t), 7.68 (1H, dd), 8.39 (1H, d), 12.58 (1H, s); m/z: ES⁺ [M+H]⁺=637.4.

### Example 174

### N-[4-(4-Chloro-3-cyano-1H-indol-7-yl)cyclohexyl]-4-[4-({4-[4-(2,4-dioxo-1,3-diazinan-1-yl)-1H-indol-1-yl]piperidin-1-yl}methyl)piperidin-1-yl]-2-fluorobenzamide

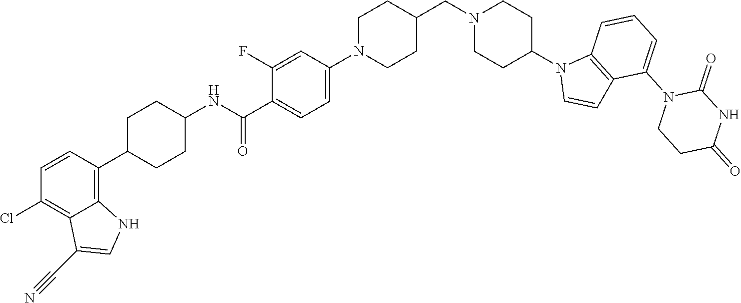 [SMILES: N#Cc1c[nH]c2c(C3CCC(NC(=O)c4ccc(N5CCC(CN6CCC(n7ccc8c(N9CCC(=O)NC9=O)cccc87)CC6)CC5)cc4F)CC3)ccc(Cl)c12]

The title compound was prepared using methodology described in example 142 using N-[4-(4-chloro-3-cyano-1H-indol-7-yl)cyclohexyl]-4-[4-(dibutoxymethyl)piperidin-1-yl]-2-fluorobenzamide and intermediate 5f to give the title compound (15 mg, 15%) as a white solid. ¹H NMR δ 1.17 (2H, d), 1.61 (4H, dt), 1.7-1.9 (5H, m), 1.98 (6H, d), 2.20 (4H, dd), 2.72-2.86 (4H, m), 3.01 (3H, t), 3.78 (2H, t), 3.86 (3H, d), 4.32-4.4 (1H, m), 6.42 (1H, d), 6.76 (2H, dd), 6.96 (1H, d), 7.08-7.22 (3H, m), 7.45-7.57 (3H, m), 7.69 (1H, dd), 8.40 (1H, s), 10.32 (1H, s), 12.57 (1H, s); m/z: ES⁺ [M+H]⁺=803.4.

### Example 175

### Intermediate 175a

### Methyl 4-[4-(dibutoxymethyl)piperidin-1-yl]-2,6-difluorobenzoate

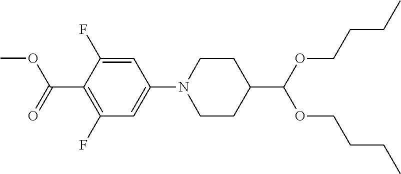 [SMILES: CCCCOC(OCCCC)C1CCN(c2cc(F)c(C(=O)OC)c(F)c2)CC1]

The title compound was prepared using methodology described in intermediate 63a using methyl 2,4,6-trifluorobenzoate and 4-(dibutoxymethyl)piperidine to give the title compound (450 mg, 20%) as a white solid. ¹H NMR δ 0.88 (6H, t), 1.14-1.56 (10H, m), 1.58-1.89 (2H, m), 2.08 (1H, s), 2.76-2.9 (2H, m), 3.29-3.44 (2H, m), 3.55 (2H, dt), 3.77 (3H, s), 3.93 (2H, d), 4.17 (1H, d), 6.64 (2H, d); m/z: ES⁺ [M+H]⁺=414.1.

### Intermediate 175b

### 4-[4-(Dibutoxymethyl)piperidin-1-yl]-2,6-difluorobenzoic acid

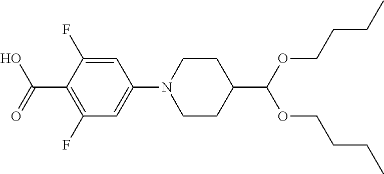 [SMILES: CCCCOC(OCCCC)C1CCN(c2cc(F)c(C(=O)O)c(F)c2)CC1]

The title compound was prepared using methodology described in intermediate 104b using methyl 4-[4-(dibutoxymethyl)piperidin-1-yl]-2,6-difluorobenzoate to give the title compound (1.6 g, 83%) as a white solid. ¹H NMR δ 0.88 (6H, td), 1.12-1.59 (10H, m), 1.69 (2H, d), 2.83 (2H, dd), 3.3-3.45 (3H, m), 3.55 (2H, dt), 3.93 (2H, d), 4.17 (1H, d), 6.64 (2H, d) 1H not observed; m/z: ES⁺ [M+H]⁺=400.0.

### Intermediate 175c

### N-[1-(4-Chloro-3-cyano-1H-indol-7-yl)piperidin-4-yl]-4-[4-(dibutoxymethyl)piperidin-1-yl]-2,6-difluorobenzamide

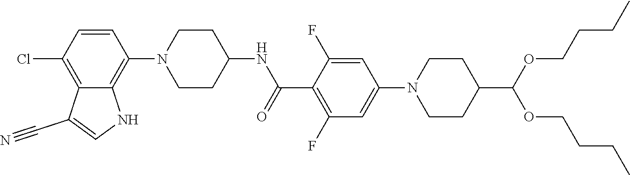 [SMILES: CCCCOC(OCCCC)C1CCN(c2cc(F)c(C(=O)NC3CCN(c4ccc(Cl)c5c(C#N)c[nH]c45)CC3)c(F)c2)CC1]

The title compound was prepared using methodology described in intermediate 153e using 4-[4-(dibutoxymethyl)piperidin-1-yl]-2,6-difluorobenzoic acid and intermediate 153d to give the title compound (100 mg, 40%) as a white solid. ¹H NMR δ 0.89 (6H, t), 1.14-1.76 (12H, m), 1.74-1.95 (5H, m), 2.66-2.82 (3H, m), 3.24-3.55 (7H, m), 3.84 (3H, d), 4.17 (1H, d), 6.62 (2H, d), 6.84 (1H, d), 7.16 (1H, d), 8.31 (1H, d), 8.47 (1H, d), 12.30 (1H, s); m/z: ES⁺ [M+H]⁺=654.0.

### Example 175

### N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2,6-difluorobenzamide

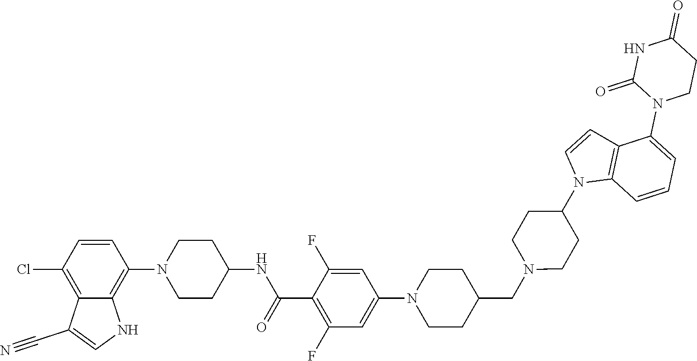 [SMILES: N#Cc1c[nH]c2c(N3CCC(NC(=O)c4c(F)cc(N5CCC(CN6CCC(n7ccc8c(N9CCC(=O)NC9=O)cccc87)CC6)CC5)cc4F)CC3)ccc(Cl)c12]

The title compound was prepared using methodology described in example 140 using N-[1-(4-chloro-3-cyano-1H-indol-7-yl)piperidin-4-yl]-4-[4-(dibutoxymethyl)piperidin-1-yl]-2,6-difluorobenzamide and intermediate 5f to give the title compound (55 mg, 43%) as a white solid. ¹H NMR δ 1.14 (2H, d), 1.75-1.88 (5H, m), 1.89-2.1 (6H, m), 2.09-2.25 (4H, m), 2.59-2.87 (6H, m), 2.99 (2H, d), 3.32 (2H, s), 3.74-3.91 (5H, m), 4.37 (1H, s), 6.42 (1H, d), 6.63 (2H, d), 6.82 (1H, d), 6.96 (1H, d), 7.14 (2H, dt), 7.47-7.57 (2H, m), 8.30 (1H, s), 8.47 (1H, d), 10.32 (1H, s), 12.30 (1H, s); m/z: ES⁺ [M+H]⁺=822.4.

### Example 176

### Intermediate 176a

### Methyl 5-[4-(dibutoxymethyl)piperidin-1-yl]pyrazine-2-carboxylate

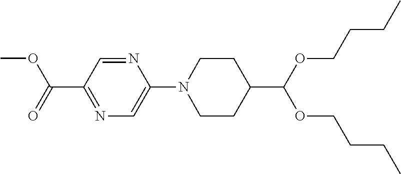 [SMILES: CCCCOC(OCCCC)C1CCN(c2cnc(C(=O)OC)cn2)CC1]

The title compound was prepared using methodology described in intermediate 11a using methyl 5-bromopyrazine-2-carboxylate and 4-(dibutoxymethyl)piperidine to give the title compound (1.0 g, 28%) as a colourless solid. ¹H NMR δ 0.87 (6H, d), 1-1.5 (12H, m), 1.84 (1H, d), 2.96 (3H, t), 3.22-3.45 (5H, m), 3.56 (3H, dt), 4.55 (1H, s), 8.36 (1H, d), 8.63 (1H, d); m/z: ES⁺ [M+H]⁺=380.1.

### Intermediate 176b

### 5-[4-(Dibutoxymethyl)piperidin-1-yl]pyrazine-2-carboxylic acid

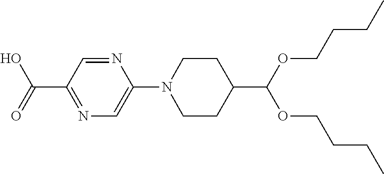 [SMILES: CCCCOC(OCCCC)C1CCN(c2cnc(C(=O)O)cn2)CC1]

The title compound was prepared using methodology described in intermediate 104b using methyl 5-[4-(dibutoxymethyl)piperidin-1-yl]pyrazine-2-carboxylate to give the title compound (640 mg, 66%) as a white solid. ¹H NMR δ 0.88 (6H, t), 1.05-1.8 (12H, m), 1.83-1.93 (1H, m), 2.61-3.42 (4H, m), 3.47-4.43 (4H, m), 4.45 (1H, s), 8.18 (1H, s), 8.59 (1H, d) 1H not observed; m/z: ES⁻[M−H]⁻=364.1.

### Intermediate 176c

### N-[1-(4-Chloro-3-cyano-1H-indol-7-yl)piperidin-4-yl]-5-[4-(dibutoxymethyl)piperidin-1-yl]pyrazine-2-carboxamide

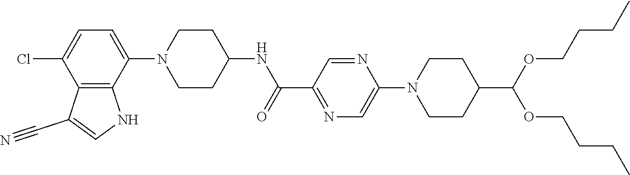 [SMILES: CCCCOC(OCCCC)C1CCN(c2cnc(C(=O)NC3CCN(c4ccc(Cl)c5c(C#N)c[nH]c45)CC3)cn2)CC1]

The title compound was prepared using methodology described in intermediate 153e using 5-[4-(dibutoxymethyl)piperidin-1-yl]pyrazine-2-carboxylic acid and intermediate 153d to give the title compound (300 mg, 88%) as a colourless gum. ¹H NMR δ 0.88 (6H, t), 1.13-1.27 (2H, m), 1.27-1.4 (4H, m), 1.42-1.54 (4H, m), 1.76 (2H, d), 1.92 (5H, s), 2.89 (8H, s), 3.39 (2H, dt), 3.56 (2H, dt), 3.94 (1H, s), 4.19 (1H, d), 6.85 (1H, d), 7.15 (1H, d), 8.21 (1H, d), 8.26 (1H, d), 8.31 (1H, s), 8.61 (1H, d), 12.29 (1H, s); m/z: ES⁺ [M+H]⁺=620.4.

### Example 176

### N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyrazine-2-carb oxamide

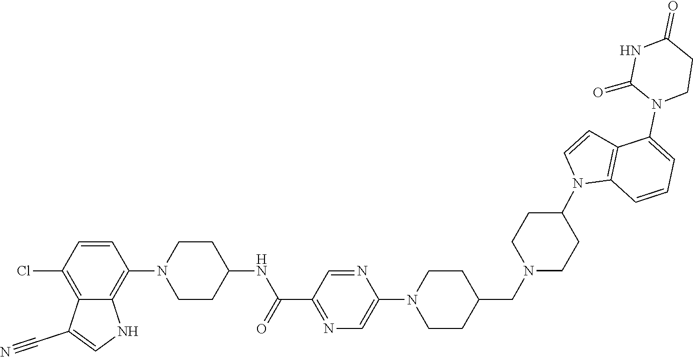 [SMILES: N#Cc1c[nH]c2c(N3CCC(NC(=O)c4cnc(N5CCC(CN6CCC(n7ccc8c(N9CCC(=O)NC9=O)cccc87)CC6)CC5)cn4)CC3)ccc(Cl)c12]

The title compound was prepared using methodology described in example 140 using N-[1-(4-chloro-3-cyano-1H-indol-7-yl)piperidin-4-yl]-5-[4-(dibutoxymethyl)piperidin-1-yl]pyrazine-2-carboxamide and intermediate 5f to give the title compound (110 mg, 58%) as a white solid. ¹H NMR δ 1.14 (2H, d), 1.79-2.01 (12H, m), 2.21 (4H, dd), 2.78 (4H, dt), 2.83-3.22 (5H, m), 3.78 (2H, t), 3.95 (1H, s), 4.39 (1H, s), 4.48 (2H, d), 6.42 (1H, d), 6.85 (1H, d), 6.96 (1H, d), 7.15 (2H, t), 7.53 (2H, dd), 8.20 (1H, d), 8.29 (2H, d), 8.62 (1H, s), 10.32 (1H, s), 12.30 (1H, s); m/z: ES⁺ [M+H]⁺=788.4.

### Example 177

### Intermediate 177a

### Methyl 5-[4-(dibutoxymethyl)piperidin-1-yl]pyrimidine-2-carboxylate

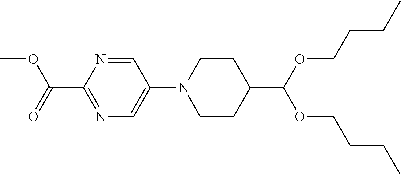 [SMILES: CCCCOC(OCCCC)C1CCN(c2cnc(C(=O)OC)nc2)CC1]

The title compound was prepared using methodology described in intermediate 63a using methyl 5-fluoropyrimidine-2-carboxylate and 4-(dibutoxymethyl)piperidine to give the title compound (2.7 g, 55%) as a white solid. ¹H NMR (CDCl₃) δ 0.91 (6H, t), 1.38 (6H, ttd), 1.47-1.63 (4H, m), 1.77-1.93 (2H, m), 1.92 (1H, d), 2.91 (2H, td), 3.42 (2H, dt), 3.62 (2H, dt), 3.86-3.97 (2H, m), 4.00 (3H, s), 4.17 (1H, d), 8.39 (2H, s); m/z: ES⁺ [M+H]⁺=380.0.

### Intermediate 177b

### 5-[4-(Dibutoxymethyl)piperidin-1-yl]pyrimidine-2-carboxylic acid

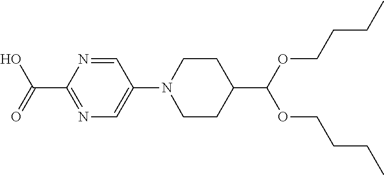 [SMILES: CCCCOC(OCCCC)C1CCN(c2cnc(C(=O)O)nc2)CC1]

The title compound was prepared using methodology described in intermediate 104b using methyl 5-[4-(dibutoxymethyl)piperidin-1-yl]pyrimidine-2-carboxylate to give the title compound (2.5 g, 96%) as a white solid. ¹H NMR δ 0.88 (6H, t), 1.26-1.38 (5H, m), 1.48 (4H, dq), 1.76 (2H, t), 2.78 (2H, td), 3.39 (2H, dt), 3.55 (4H, dt), 3.94 (2H, d), 4.19 (1H, d), 8.43 (2H, s) 1H not observed; m/z: ES⁺ [M+H]⁺=366.1.

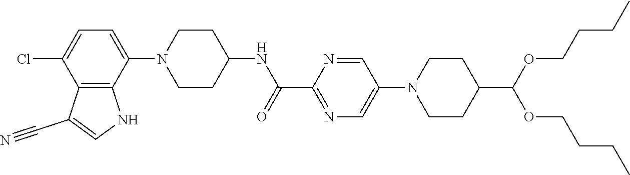 [SMILES: CCCCOC(OCCCC)C1CCN(c2cnc(C(=O)NC3CCN(c4ccc(Cl)c5c(C#N)c[nH]c45)CC3)nc2)CC1]

The title compound was prepared using methodology described in example 166c using 5-[4-(dibutoxymethyl)piperidin-1-yl]pyrimidine-2-carboxylic acid and intermediate 153d to give the title compound (250 mg, 55%) as a white solid. ¹H NMR δ 0.94 (6H, d), 1.29 (4H, d), 1.41-1.84 (18H, m), 1.97 (2H, d), 2.85 (2H, s), 3.54 (4H, d), 4.12 (2H, d), 5.76 (1H, s), 6.86 (1H, d), 7.16 (1H, d), 8.49 (2H, d), 12.32 (1H, s); m/z: ES⁺ [M+H]⁺=622.1.

### Example 177

### N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyrimidine-2-carboxamide

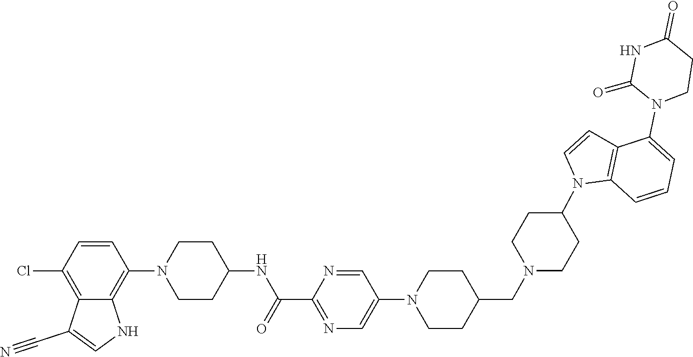 [SMILES: N#Cc1c[nH]c2c(N3CCC(NC(=O)c4ncc(N5CCC(CN6CCC(n7ccc8c(N9CCC(=O)NC9=O)cccc87)CC6)CC5)cn4)CC3)ccc(Cl)c12]

The title compound was prepared using methodology described in example 140 using N-[1-(4-chloro-3-cyano-1H-indol-7-yl)piperidin-4-yl]-5-[4-(dibutoxymethyl)piperidin-1-yl]pyrimidine-2-carboxamide and intermediate 5f to give the title compound (14 mg, 4%) as a white solid. ¹H NMR δ 1.22 (3H, s), 1.89 (11H, d), 2.12-2.29 (4H, m), 2.69-3.07 (9H, m), 3.78 (2H, d), 3.98 (3H, t), 4.38 (1H, s), 6.42 (1H, s), 6.77-7.01 (2H, m), 7.14 (2H, d), 7.52 (2H, d), 8.29 (1H, s), 8.42 (1H, s), 8.54 (2H, s), 10.32 (1H, s), 12.35 (1H, s); m/z: ES⁺ [M+H]⁺=788.3.

### Example 178

### Intermediate 178a

### Methyl 4-[4-(dibutoxymethyl)piperidin-1-yl]-2-methylbenzoate

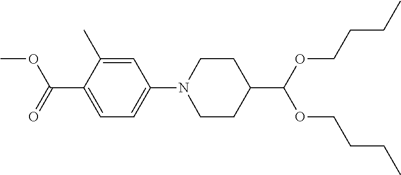 [SMILES: CCCCOC(OCCCC)C1CCN(c2ccc(C(=O)OC)c(C)c2)CC1]

The title compound was prepared using methodology described in intermediate 63a using methyl 4-fluoro-2-methylbenzoate and 4-(dibutoxymethyl)piperidine to give the title compound (1.7 g, 36%) as a white solid. ¹H NMR (CDCl₃) δ 0.93 (6H, t), 1.3-1.65 (10H, m), 1.75-1.91 (3H, m), 2.58 (3H, s), 2.71-2.86 (2H, m), 3.43 (2H, dt), 3.63 (2H, dt), 3.83 (3H, s), 3.82-3.93 (1H, m), 4.17 (1H, d), 6.70 (2H, d), 7.87 (1H, d); m/z: ES⁺ [M+H]⁺=392.0.

### Intermediate 178b

### 4-[4-(Dibutoxymethyl)piperidin-1-yl]-2-methylbenzoic acid

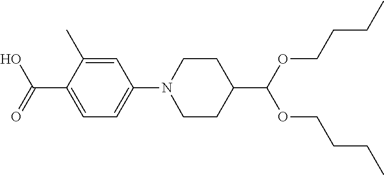 [SMILES: CCCCOC(OCCCC)C1CCN(c2ccc(C(=O)O)c(C)c2)CC1]

The title compound was prepared using methodology described in intermediate 104b using methyl 4-[4-(dibutoxymethyl)piperidin-1-yl]-2-methylbenzoate to give the title compound (1.3 g, 79%) as a white solid. ¹H NMR (CDCl₃) δ 0.93 (6H, t), 1.3-1.48 (4H, m), 1.45-1.67 (4H, m), 1.88 (2H, s), 1.99 (3H, s), 2.64 (3H, s), 3.01 (2H, s), 3.45 (2H, dt), 3.66 (2H, dt), 3.85 (2H, d), 4.24 (1H, d), 7.11 (2H, s), 8.05 (1H, d) 1H not observed; m/z: ES⁺ [M+H]⁺=378.1.

### Intermediate 178c

### N-[1-(4-Chloro-3-cyano-1H-indol-7-yl)piperidin-4-yl]-4-[4-(dibutoxymethyl)piperidin-1-yl]-2-methylbenzamide

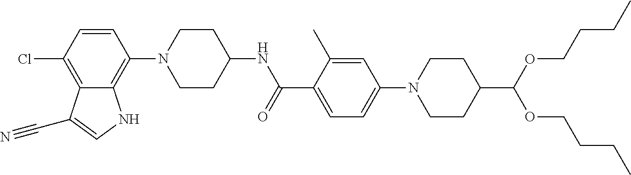 [SMILES: CCCCOC(OCCCC)C1CCN(c2ccc(C(=O)NC3CCN(c4ccc(Cl)c5c(C#N)c[nH]c45)CC3)c(C)c2)CC1]

The title compound was prepared using methodology described in example 166c using 4-[4-(dibutoxymethyl)piperidin-1-yl]-2-methylbenzoic acid and intermediate 153d to give the title compound (147 mg, 58%) as a white solid. ¹H NMR δ 0.89 (5H, t), 1.33 (6H, dt), 1.49 (4H, p), 1.72 (3H, d), 1.84 (1H, d), 1.92 (4H, s), 2.34 (3H, s), 2.52 (2H, s), 2.65 (2H, t), 2.79 (2H, t), 3.39 (2H, dt), 3.49-3.63 (2H, m), 3.78 (2H, d), 3.88 (1H, s), 4.18 (1H, d), 6.75 (2H, d), 6.85 (1H, d), 7.16 (1H, d), 7.23 (1H, d), 8.05 (1H, d), 8.31 (1H, s), 12.31 (1H, s); m/z: ES⁺ [M+H]⁺=634.4.

### Example 178: N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-methylbenzamide

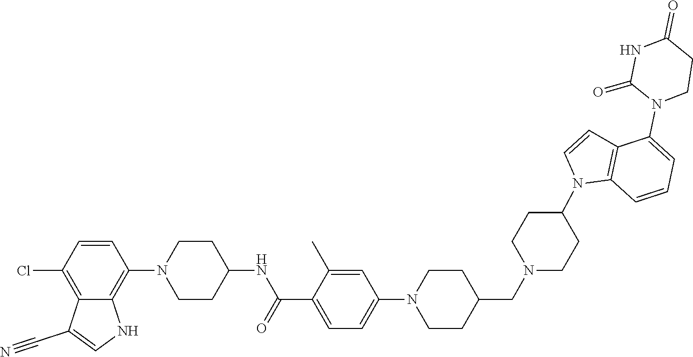 [SMILES: Cc1cc(N2CCC(CN3CCC(n4ccc5c(N6CCC(=O)NC6=O)cccc54)CC3)CC2)ccc1C(=O)NC1CCN(c2ccc(Cl)c3c(C#N)c[nH]c23)CC1]

The title compound was prepared using methodology described in example 140 using N-[1-(4-chloro-3-cyano-1H-indol-7-yl)piperidin-4-yl]-4-[4-(dibutoxymethyl)piperidin-1-yl]-2-methylbenzamide and intermediate 5f to give the title compound (40 mg, 22%) as a white solid. ¹H NMR δ 1.16-1.26 (2H, m), 1.73 (1H, s), 1.84 (4H, q), 1.96 (6H, td), 2.11-2.28 (4H, m), 2.34 (3H, s), 2.66-2.82 (6H, m), 3.00 (2H, d), 3.37 (2H, s), 3.74-3.82 (4H, m), 3.88 (1H, s), 4.38 (1H, s), 6.42 (1H, d), 6.76 (2H, d), 6.84 (1H, d), 6.96 (1H, d), 7.15 (2H, dt), 7.23 (1H, d), 7.52 (2H, dd), 8.04 (1H, d), 8.30 (1H, s), 10.32 (1H, s), 12.31 (1H, s); m/z: ES⁺ [M+H]⁺=800.0.

### Example 179

### Intermediate 179a

### Methyl 6-[4-(dibutoxymethyl)piperidin-1-yl]pyridazine-3-carboxylate

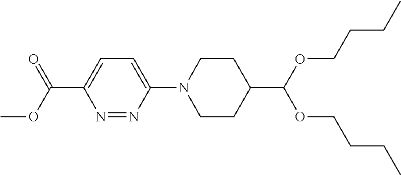 [SMILES: CCCCOC(OCCCC)C1CCN(c2ccc(C(=O)OC)nn2)CC1]

The title compound was prepared using methodology described in intermediate 11a using methyl 6-chloropyridazine-3-carboxylate and 4-(dibutoxymethyl)piperidine to give the title compound (6.5 g, 59%) as a white solid. ¹H NMR δ 0.88 (6H, t), 1.12-1.26 (2H, m), 1.29-1.4 (4H, m), 1.42-1.55 (4H, m), 1.72-1.8 (2H, m), 1.86-1.98 (1H, m), 2.91-3.03 (2H, m), 3.33-3.44 (2H, m), 3.51-3.61 (2H, m), 3.86 (3H, s), 4.18 (1H, d), 4.54 (2H, d), 7.27 (1H, d), 7.80 (1H, d); m/z: ES⁺ [M+H]⁺=380.3.

### Intermediate 179b

### 6-[4-(Dibutoxymethyl)piperidin-1-yl]pyridazine-3-carboxylic acid

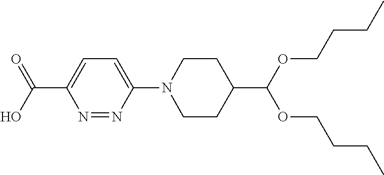 [SMILES: CCCCOC(OCCCC)C1CCN(c2ccc(C(=O)O)nn2)CC1]

The title compound was prepared using methodology described in intermediate 104b using methyl 6-[4-(dibutoxymethyl)piperidin-1-yl]pyridazine-3-carboxylate to give the title compound (3.2 g, 51%) as a yellow solid. ¹H NMR δ 0.87 (6H, t), 1.14-1.41 (6H, m), 1.41-1.58 (4H, m), 1.69-1.81 (2H, m), 1.85-1.95 (1H, m), 2.96 (2H, t), 3.31-3.45 (2H, m), 3.48-3.62 (2H, m), 4.18 (1H, d), 4.53 (2H, d), 7.26 (1H, d), 7.78 (1H, d); m/z: ES⁺ [M+H]⁺=366.3.

### Intermediate 179c

### N-[1-(4-Chloro-3-cyano-1H-indol-7-yl)piperidin-4-yl]-6-[4-(dibutoxymethyl)piperidin-1-yl]pyridazine-3-carboxamide

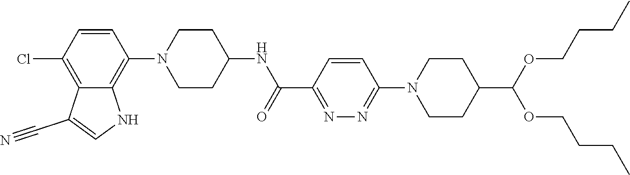 [SMILES: CCCCOC(OCCCC)C1CCN(c2ccc(C(=O)NC3CCN(c4ccc(Cl)c5c(C#N)c[nH]c45)CC3)nn2)CC1]

The title compound was prepared using methodology described in intermediate 166c using 6-[4-(dibutoxymethyl)piperidin-1-yl]pyridazine-3-carboxylic acid and intermediate 153d to give the title compound (1.0 g, 88%) as a white solid. ¹H NMR δ 0.88 (6H, t), 1.2-1.42 (4H, m), 1.42-1.56 (4H, m), 1.76 (2H, d), 1.94 (5H, s), 2.01 (1H, d), 2.74 (9H, d), 3.49-3.63 (2H, m), 3.98 (1H, s), 4.19 (1H, d), 4.52 (2H, d), 6.86 (1H, d), 7.16 (1H, d), 7.35 (1H, d), 7.83 (1H, d), 8.32 (1H, d), 8.73 (1H, d), 12.34 (1H, s); m/z: ES⁺ [M+H]⁺=622.5.

### Example 179

### N-[1-(4-Chloro-3-cyano-1H-indol-7-yl)piperidin-4-yl]-6-[4-({4-[4-(2,4-dioxo-1,3-diazinan-1-yl)-1H-indol-1-yl]piperidin-1-yl}methyl)piperidin-1-yl]pyridazine-3-carboxamide

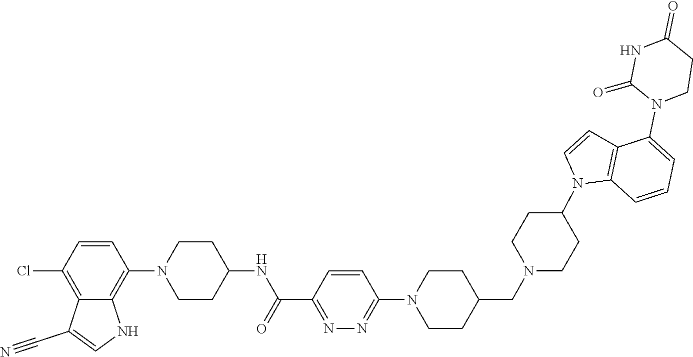 [SMILES: N#Cc1c[nH]c2c(N3CCC(NC(=O)c4ccc(N5CCC(CN6CCC(n7ccc8c(N9CCC(=O)NC9=O)cccc87)CC6)CC5)nn4)CC3)ccc(Cl)c12]

The title compound was prepared using methodology described in example 140 using N-[1-(4-chloro-3-cyano-1H-indol-7-yl)piperidin-4-yl]-6-[4-(dibutoxymethyl)piperidin-1-yl]pyridazine-3-carboxamide and intermediate 5f to give the title compound (53 mg, 21%) as a white solid. ¹H NMR δ 1.18 (2H, dd), 1.77-2.06 (11H, m), 2.21 (4H, dd), 2.50 (4H, q), 2.72-2.86 (4H, m), 3.03 (4H, t), 3.37 (2H, s), 3.78 (2H, t), 3.98 (1H, d), 4.38 (1H, s), 4.50 (2H, d), 6.42 (1H, d), 6.86 (1H, d), 6.96 (1H, d), 7.15 (2H, t), 7.35 (1H, d), 7.48-7.57 (2H, m), 7.83 (1H, d), 8.31 (1H, d), 8.73 (1H, d), 10.33 (1H, s), 12.33 (1H, s); m/z: ES⁺ [M+H]⁺=788.3.

### Example 180

### Intermediate 180a

### 4-Chloro-7-(1,4-dioxa-7-azaspiro[4.5]decan-7-yl)-1H-indole-3-carbonitrile

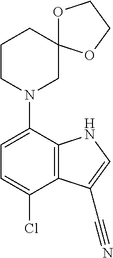 [SMILES: N#Cc1c[nH]c2c(N3CCCC4(C3)OCCO4)ccc(Cl)c12]

The title compound was prepared using methodology described in intermediate 11c using intermediate 1b and 1,4-dioxa-7-azaspiro[4.5]decane to give the title compound (1.5 g, 36%) as a yellow solid. ¹H NMR δ 1.70 (3H, t), 1.91 (2H, tt), 2.97 (2H, s), 3.88-3.91 (5H, m), 6.78 (1H, d), 7.08-7.32 (1H, m), 8.28 (1H, s), 12.15 (1H, s); m/z: ES⁺ [M+H]⁺=317.9.

### Intermediate 180b

### 4-Chloro-7-(3-oxopiperidin-1-yl)-1H-indole-3-carbonitrile

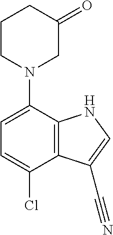 [SMILES: N#Cc1c[nH]c2c(N3CCCC(=O)C3)ccc(Cl)c12]

4-Chloro-7-(1,4-dioxa-7-azaspiro[4.5]decan-7-yl)-1H-indole-3-carbonitrile (1.5 g, 4.72 mmol) was stirred in formic acid (15 mL) at 40° C. for 16 h. The reaction was then cooled to RT and the solvent was evaporated to dryness to afford the title compound (0.900 g, 69.7%) as a black gum which was used without further purification. ¹H NMR δ 0.66-0.87 (1H, m), 1.36 (1H, d), 2.94-3.08 (2H, m), 3.20 (1H, td), 3.38 (1H, d), 4.33 (2H, s), 8.15 (2H, s), 8.26 (1H, s), 8.42 (1H, s); m/z: ES⁺ [M+H]⁺=274.1.

### Intermediate 180c

### 7-(3-Aminopiperidin-1-yl)-4-chloro-1H-indole-3-carbonitrile

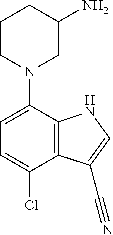 [SMILES: N#Cc1c[nH]c2c(N3CCCC(N)C3)ccc(Cl)c12]

The title compound was prepared using methodology described in intermediate 152e using 4-chloro-7-(3-oxopiperidin-1-yl)-1H-indole-3-carbonitrile to give the title compound (600 mg, 74%) as a white solid. ¹H NMR δ 1.71-1.8 (1H, m), 1.77-1.9 (2H, m), 2.42 (1H, q), 2.62 (1H, t), 3.18 (3H, d), 3.23-3.32 (1H, m), 6.75 (1H, d), 7.11 (1H, d), 8.29 (1H, s) 3H not observed; m/z: ES⁺ [M+H]⁺=275.2.

### Intermediates 180c and 181a

7-(3-Aminopiperidin-1-yl)-4-chloro-1H-indole-3-carbonitrile (150 mg, 0.41 mmol) was added to DIPEA (0.216 mL, 1.23 mmol), HATU (313 mg, 0.82 mmol) and intermediate 170b (113 mg, 0.41 mmol) in DMF (5 mL) and was stirred at RT for 2 h. The solvent was evaporated to dryness to afford crude product and was purified by flash silica chromatography, elution gradient 0 to 20% EtOAc in Et₂O to give the title compound as a racemic mixture. The enantiomers were separated by preparative chiral-HPLC using conditions: CHIRALPAK ID, 2*25 cm, 5 m, Hex:DCM (3:1, 0.5% 2M NH3-MeOH) and EtOH to give, in order of elution, intermediate 180c (isomer 1, 100 mg, 39%) and intermediate 181a (isomer 2, 100 mg, 39%) as yellow solids.

### Intermediate 180c

### N-[(3R*)-1-(4-Chloro-3-cyano-1H-indol-7-yl)piperidin-3-yl]-6-[4-(dibutoxymethyl)piperidin-1-yl]pyridine-2-carboxamide [Absolute Stereochemistry not Yet Confirmed]

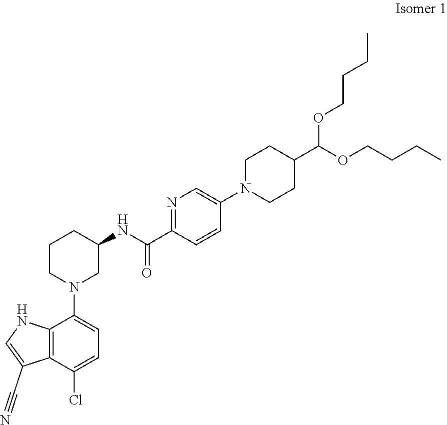 [SMILES: CCCCOC(OCCCC)C1CCN(c2ccc(C(=O)N[C@@H]3CCCN(c4ccc(Cl)c5c(C#N)c[nH]c45)C3)nc2)CC1]

¹H NMR δ 0.88 (6H, t), 1.13-1.56 (10H, m), 1.62 (1H, d), 1.74 (2H, d), 1.94 (3H, d), 2.81 (4H, q), 3.15 (1H, d), 3.31 (1H, s), 3.39 (2H, dt), 3.56 (2H, dt), 3.93 (2H, d), 4.20 (2H, t), 4.42 (OH, s), 6.84 (1H, d), 7.15 (1H, d), 7.38 (1H, dd), 7.83 (1H, d), 8.22-8.38 (3H, m), 12.34 (1H, d); m/z: ES⁺ [M+H]⁺=621.0; >99% ee.

### Intermediate 181a

### N-[(3S*)-1-(4-Chloro-3-cyano-1H-indol-7-yl)piperidin-3-yl]-6-[4-(dibutoxymethyl)piperidin-1-yl]pyridine-2-carboxamide [Absolute Stereochemistry not Yet Confirmed]

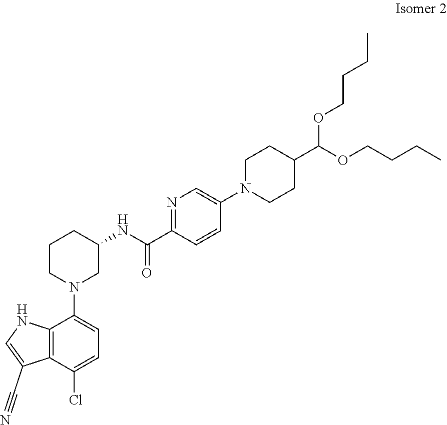 [SMILES: CCCCOC(OCCCC)C1CCN(c2ccc(C(=O)N[C@H]3CCCN(c4ccc(Cl)c5c(C#N)c[nH]c45)C3)nc2)CC1]

¹H NMR δ 0.88 (6H, t), 1.13-1.56 (10H, m), 1.62 (1H, d), 1.74 (2H, d), 1.94 (3H, d), 2.81 (4H, q), 3.15 (1H, d), 3.31 (1H, s), 3.39 (2H, dt), 3.56 (2H, dt), 3.93 (2H, d), 4.20 (2H, t), 4.42 (OH, s), 6.84 (1H, d), 7.15 (1H, d), 7.38 (1H, dd), 7.83 (1H, d), 8.22-8.38 (3H, m), 12.34 (1H, d); m/z: ES⁺ [M+H]⁺=621.0; >99% cc.

### Example 180

### N-[(3R*)-1-(4-Chloro-3-cyano-1H-indol-7-yl)piperidin-3-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide [Absolute Stereochemistry not Yet Confirmed]

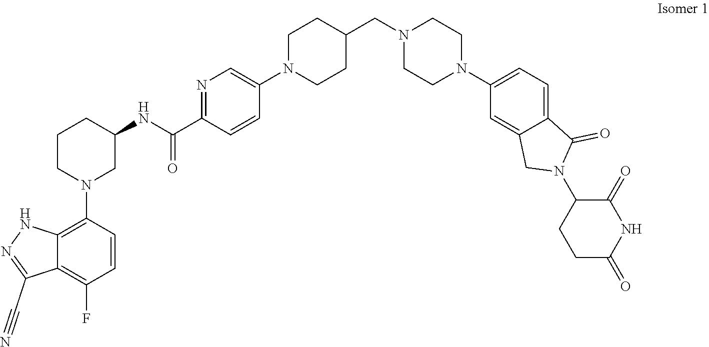 [SMILES: N#Cc1n[nH]c2c(N3CCC[C@@H](NC(=O)c4ccc(N5CCC(CN6CCN(c7ccc8c(c7)CN(C7CCC(=O)NC7=O)C8=O)CC6)CC5)cn4)C3)ccc(F)c12]

The title compound was prepared using methodology described in example 140 using intermediate 180c and intermediate 1i to give the title compound (40 mg, 31%) as a white solid. ¹H NMR δ 1.19 (2H, d), 1.63 (OH, s), 1.80 (2H, s), 1.83 (2H, s), 1.88 (4H, s), 2.20 (2H, d), 2.36 (1H, d), 2.58 (1H, d), 2.85 (6H, s), 3.15 (1H, s), 3.30 (7H, d), 3.90 (2H, d), 4.20 (2H, d), 4.33 (1H, d), 5.05 (1H, dd), 6.81 (1H, d), 7.05 (2H, d), 7.12 (1H, d), 7.38 (1H, d), 7.52 (1H, d), 7.83 (1H, d), 8.23-8.33 (3H, m), 10.95 (1H, s), 12.35 (1H, s); m/z: ES⁺ [M+H]=803.4.

### Example 181

### N-[(3S*)-1-(4-Chloro-3-cyano-1H-indol-7-yl)piperidin-3-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide [Absolute Stereochemistry not Yet Confirmed]

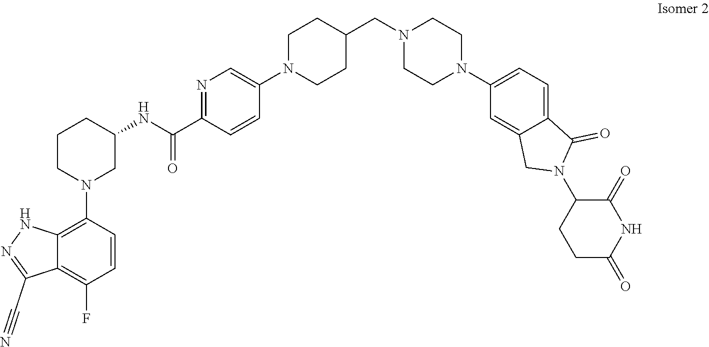 [SMILES: N#Cc1n[nH]c2c(N3CCC[C@H](NC(=O)c4ccc(N5CCC(CN6CCN(c7ccc8c(c7)CN(C7CCC(=O)NC7=O)C8=O)CC6)CC5)cn4)C3)ccc(F)c12]

The title compound was prepared using methodology described in example 140 using intermediate 181a and intermediate 1i to give the title compound (48 mg, 37%) as a white solid. ¹H NMR δ 1.19 (2H, d), 1.62 (1H, s), 1.81 (4H, d), 1.88 (2H, s), 1.97 (2H, s), 2.20 (2H, d), 2.37 (1H, d), 2.58 (2H, d), 2.83 (5H, t), 3.15 (1H, s), 3.30 (7H, d), 3.90 (2H, d), 4.20 (2H, d), 4.33 (1H, d), 5.05 (1H, dd), 6.82 (1H, d), 7-7.17 (3H, m), 7.35-7.42 (1H, m), 7.52 (1H, d), 7.83 (1H, d), 8.23-8.34 (3H, m), 10.95 (1H, s), 12.35 (1H, s); m/z: ES⁺ [M+H]⁺=803.3.

### LNCaP Androgen Receptor Imaging Assay

This cell-based imaging assay uses immuno-fluorescence to measure endogenous nuclear AR staining in the prostate cancer cell line LNCaP (expressing AR-full length). The purpose of the assay is to identify compounds that regulate AR protein level according to the protocol laid out in the steps below.

All work was performed in a sterile tissue culture hood up to the point of cell fixation. LNCaP cells were cultured at 6-8×10\hat{ }6 per T175 flask and split every 3-4 days. Medium: RPMI (R7638, VWR Sigma) containing 10% FCS and 1% glutamine. Cells were plated in black wall transparent bottom plates at a density of 12,000 cells (Cedex™ Cell counter, Beckmann & Coulter), 40 μL per well, using a Multidrop (ThermoFisher).

Cells were dosed with test compound diluted in DMSO using an Echo 555/655™ (Labcyte) and the final concentration of DMSO in the well was 0.3%. A neutral control (DMSO) and a positive AR degrader control at 3 μM (a prior art AR PROTAC) were included. The Echo uses acoustic technology to perform direct microplate-to-microplate transfers of DMSO compound solutions to assay plates. The system can be programmed to transfer volumes as low as 2.5 nL in multiple increments between microplates and in so doing generates a serial dilution of compound in the assay plate which is then back-filled to normalise the DMSO concentration across the dilution range. Compounds were dispensed as a 12 point dose response with indicated concentrations. The plates were incubated at 37° C./5% CO2 for 24 h and then fixed and stained as below. 40 μL of 8% paraformaldehyde solution (v/v) in phosphate buffered saline (PBS) was added on top of the media already in the well using a Multidrop to fix plates giving a final concentration of 4% PFA (v/v) for 20-30 minutes (keep in fume hood to minimise PFA exposure). After 20-30 mins, the plates were washed with 3×PBS using a BioTek washer in a fume hood. A shake out of remaining volume in the well was performed to ensure best removal of remaining PBS. At this plates could be stored in PBS after washing for at least 3 days at 4° C., if so, PBS was removed by the BioTek before continuing with the next step.

The cells were incubated with a Mouse monoclonal Anti-AR antibody AR441 (DAKO M3562) (1:1000), diluted in modified blocking buffer (PBS+1% BSA+0.1% TritonX); 15 μL/well for 2 h at RT. The plates were then washed with 3×PBS using a BioTek washer. A shake out of remaining volume in the well was performed to ensure best removal of remaining PBS. Cells were then incubated with Goat anti-mouse secondary antibody 488 (Invitrogen A11001) (1:500) and nuclear stain, DRAQ5 (Abcam Ab108410) (1:2000) diluted in modified blocking buffer for 45 mins at room temperature 15 μL/well. Plates are then washed with 3×PBS using a BioTek washer, without final aspiration, and sealed with black plate seals. Plates could be stored after staining for three days at 4° C. before read if required.

Plates are read using a Cellomics Cellinsight to measure the Androgen Receptor level in each well. The data is exported into Genedata to perform curve fitting analysis. All data are normalized as % of positive control and the BellFit method automatically determines the correct fit method based on the presence of hook effect, choosing the BellFit when there is a hook and HillFit when there is no hook. Down-regulation of AR is expressed as a DC50 value and determined by calculation of the concentration of compound that is required to give 50% of maximum degradation of AR as per the curve fit for that compound. The maximum degradation observed for each compound is expressed as Dmax. The AR degradation data for compounds is shown in the table below and may be a result from a single experiment or an average of two or more experiments.

### LNCaP Androgen Receptor L702H Imaging Assay

While treatment with AR antagonists has been shown to be of patient benefit, most cancers eventually progress to castration resistant prostate cancer (CRPC). At this stage, anti-androgen therapy is largely ineffective due to AR amplification, overexpression or point mutation. One of the point mutations observed in patient populations is L702H in the AR Ligand Binding Domain. LNCAP cells have been engineered to introduce the mutation L702H into the AR gene using CRISPR/Cas9. The previously described imaging-based AR assay will monitor nuclear AR (L702H) levels in these cells to identify compounds that regulate AR (L702H). Down-regulation of AR (L702H) is expressed as a DC50 value and determined by calculation of the concentration of compound that is required to give 50% of maximum degradation of AR (L702H) as per the curve fit for that compound. The maximum degradation observed for each compound is expressed as Dmax. The AR and AR (L702H) degradation data for compounds is shown in the table below and may be a result from a single experiment or an average of two or more experiments.

| | | | LNCaP AR | LNCaP AR |
|---|---|---|---|---|
| | LNCaP AR | LNCaP AR | (L702H) | (L702H) |
| | Degradation DC₅₀ | Degradation Dₘₐₓ | Degradation DC₅₀ | Degradation Dₘₐₓ |
| Example No. | (μM) | (%) | (μM) | (%) |
| 1 | 0.006 | 99 | 0.006 | 100 |
| 2 | 0.006 | 96 | 0.005 | 101 |
| 3 | 0.018 | 85 | 0.017 | 99 |
| 4 | 0.006 | 96 | 0.005 | 106 |
| 5 | 0.008 | 103 | 0.012 | 110 |
| 6 | 0.010 | 100 | 0.013 | 103 |
| 7 | 0.009 | 98 | 0.014 | 101 |
| 8 | 0.029 | 99 | 0.103 | 96 |
| 9 | 0.007 | 105 | 0.009 | 106 |
| 10 | 0.007 | 96 | 0.005 | 103 |
| 11 | 0.009 | 102 | 0.018 | 105 |
| 12 | 0.006 | 95 | 0.012 | 97 |
| 13 | 0.007 | 107 | 0.006 | 109 |
| 14 | 0.006 | 107 | 0.009 | 112 |
| 15 | 0.009 | 97 | 0.024 | 85 |
| 16 | 0.012 | 77 | 0.024 | 84 |
| 17 | 0.003 | 100 | 0.004 | 102 |
| 18 | 0.014 | 93 | 0.018 | 96 |
| 19 | 0.004 | 102 | 0.005 | 102 |
| 20 | 0.009 | 94 | 0.008 | 101 |
| 21 | 0.008 | 92 | 0.007 | 103 |
| 22 | 0.014 | 80 | 0.023 | 81 |
| 23 | 0.012 | 100 | 0.057 | 94 |
| 24 | 0.006 | 100 | 0.018 | 83 |
| 25 | 0.006 | 92 | 0.004 | 104 |
| 26 | 0.009 | 92 | 0.007 | 106 |
| 27 | 0.014 | 99 | 0.027 | 75 |
| 28 | 0.005 | 96 | 0.023 | 83 |
| 29 | 0.079 | 94 | 0.060 | 74 |
| 30 | 0.025 | 98 | 0.083 | 83 |
| 31 | 0.008 | 94 | 0.022 | 88 |
| 32 | 0.005 | 99 | 0.010 | 100 |
| 33 | 0.012 | 97 | 0.033 | 90 |
| 34 | 0.007 | 98 | 0.012 | 102 |
| 35 | 0.019 | 84 | 0.029 | 91 |
| 36 | 0.005 | 102 | 0.007 | 102 |
| 37 | 0.015 | 90 | 0.011 | 100 |
| 38 | 0.009 | 97 | 0.012 | 95 |
| 39 | 0.016 | 88 | 0.033 | 99 |
| 40 | 0.007 | 105 | 0.012 | 106 |
| 41 | 0.017 | 98 | 0.029 | 95 |
| 42 | 0.029 | 101 | 0.062 | 107 |
| 43 | 0.073 | 94 | 0.160 | 97 |
| 44 | 0.004 | 95 | 0.002 | 96 |
| 45 | 0.003 | 96 | 0.006 | 95 |
| 46 | 0.004 | 101 | 0.012 | 98 |
| 47 | 0.007 | 84 | 0.007 | 100 |
| 48 | 0.057 | 97 | 0.359 | 99 |
| 49 | 0.033 | 91 | 0.045 | 97 |
| 50 | 0.137 | 98 | 0.769 | 94 |
| 51 | 0.037 | 101 | 0.071 | 96 |
| 52 | 0.062 | 93 | 0.052 | 97 |
| 53 | 0.030 | 99 | 0.078 | 103 |
| 54 | 0.036 | 77 | 0.052 | 97 |
| 55 | 0.033 | 99 | 0.034 | 103 |
| 56 | 0.061 | 101 | 0.357 | 94 |
| 57 | 0.018 | 87 | 0.031 | 79 |
| 58 | 0.013 | 96 | 0.114 | 78 |
| 59 | 0.051 | 88 | 0.047 | 95 |
| 60 | 0.005 | 93 | 0.023 | 57 |
| 61 | 0.006 | 94 | 0.025 | 65 |
| 62 | 0.007 | 99 | 0.080 | 71 |
| 63 | 0.006 | 94 | 0.016 | 63 |
| 64 | 0.005 | 83 | 0.008 | 56 |
| 65 | 0.009 | 96 | 0.798 | 78 |
| 66 | 0.010 | 97 | 0.022 | 94 |
| 67 | 0.004 | 98 | 0.014 | 87 |
| 68 | 0.006 | 97 | 0.021 | 84 |
| 69 | 0.009 | 92 | 0.005 | 94 |
| 70 | 0.009 | 97 | 0.009 | 101 |
| 71 | 0.007 | 84 | 0.007 | 94 |
| 72 | 0.014 | 91 | 0.021 | 92 |
| 73 | 0.007 | 93 | 0.013 | 90 |
| 74 | 0.008 | 93 | 0.021 | 70 |
| 75 | 0.062 | 68 | 0.068 | 83 |
| 76 | 0.018 | 92 | 0.038 | 104 |
| 77 | 0.014 | 79 | 0.067 | 69 |
| 78 | 1.37 | 70 | 3.1 | 100 |
| 79 | 0.009 | 92 | 0.021 | 79 |
| 80 | 0.038 | 64 | 3.1 | 100 |
| 81 | >3 | — | 3.1 | 100 |
| 82 | 0.031 | 90 | 0.022 | 91 |
| 83 | 3.1 | 100 | >3 | — |
| 84 | 0.022 | 99 | 0.012 | 103 |
| 85 | 0.004 | 98 | 0.017 | 83 |
| 86 | 0.015 | 94 | 0.014 | 99 |
| 87 | 0.005 | 95 | 0.008 | 94 |
| 88 | 0.013 | 96 | 0.021 | 99 |
| 89 | 0.421 | 70 | 0.056 | 97 |
| 90 | 0.281 | 87 | 0.164 | 101 |
| 91 | 0.023 | 100 | 0.010 | 99 |
| 92 | 3.1 | 100 | 3.1 | 100 |
| 93 | 0.020 | 87 | 0.011 | 95 |
| 94 | >3 | — | — | — |
| 95 | 0.010 | 54 | >3 | — |
| 96 | 0.031 | 54 | 0.012 | 95 |
| 97 | >3 | — | >3 | — |
| 98 | 0.090 | 40 | 0.015 | 83 |
| 99 | >3 | — | 3.1 | 100 |
| 100 | 0.029 | 101 | 0.449 | 89 |
| 101 | 0.006 | 97 | 0.015 | 87 |
| 102 | 0.052 | 86 | 0.044 | 106 |
| 103 | 0.008 | 85 | 0.007 | 107 |
| 104 | 0.013 | 98 | 0.046 | 97 |
| 105 | 0.061 | 89 | 0.049 | 95 |
| 106 | 0.022 | 97 | 0.050 | 93 |
| 107 | 0.006 | 93 | 0.300 | 107 |
| 108 | 0.007 | 104 | 0.034 | 102 |
| 109 | 0.005 | 97 | 0.010 | 96 |
| 110 | 0.006 | 88 | 0.005 | 97 |
| 111 | 0.004 | 99 | 0.005 | 106 |
| 112 | >3 | — | 0.017 | 87 |
| 113 | 0.019 | 92 | 0.036 | 76 |
| 114 | 0.019 | 101 | 0.031 | 101 |
| 115 | 0.014 | 99 | 0.013 | 102 |
| 116 | 0.011 | 103 | 0.023 | 97 |
| 117 | 0.017 | 89 | 0.363 | 84 |
| 118 | 0.024 | 92 | 2.3 | 100 |
| 119 | 0.006 | 99 | 0.014 | 103 |
| 120 | 0.013 | 101 | 0.040 | 90 |
| 121 | 0.018 | 81 | 3.1 | 98 |
| 122 | 0.093 | 85 | 0.067 | 75 |
| 123 | 0.014 | 95 | 0.018 | 104 |
| 124 | 0.003 | 95 | 0.009 | 93 |
| 125 | 0.004 | 102 | 0.009 | 93 |
| 126 | 0.004 | 98 | 0.002 | 107 |
| 127 | 0.004 | 100 | 0.004 | 110 |
| 128 | 0.026 | 102 | 0.061 | 103 |
| 129 | 0.020 | 95 | 0.029 | 99 |
| 130 | 0.031 | 99 | 0.042 | 78 |
| 131 | 0.017 | 105 | 0.041 | 94 |
| 132 | 0.006 | 98 | 0.005 | 115 |
| 133 | 0.004 | 103 | 0.008 | 97 |
| 134 | 0.005 | 106 | 0.022 | 94 |
| 135 | 0.024 | 105 | 0.081 | 91 |
| 136 | 0.010 | 104 | 0.014 | 108 |
| 137 | 0.010 | 105 | 0.020 | 109 |
| 138 | 0.008 | 104 | 0.006 | 108 |
| 139 | 0.010 | 105 | 0.016 | 107 |
| 140 | 0.007 | 88 | 0.014 | 56 |
| 141 | 0.021 | 95 | 0.023 | 105 |
| 142 | 0.025 | 106 | 0.045 | 109 |
| 143 | 0.037 | 101 | 0.067 | 104 |
| 144 | 0.021 | 97 | 0.095 | 75 |
| 145 | 0.009 | 98 | 0.040 | 70 |
| 146 | 0.027 | 100 | 0.038 | 102 |
| 147 | 0.006 | 102 | 0.038 | 96 |
| 148 | 0.020 | 99 | 0.052 | 89 |
| 149 | 0.020 | 96 | 0.060 | 91 |
| 150 | 0.014 | 82 | 0.893 | 50 |
| 151 | 0.018 | 67 | 0.497 | 42 |
| 152 | 0.002 | 105 | 0.002 | 108 |
| 153 | 0.002 | 109 | 0.003 | 114 |
| 154 | 0.004 | 81 | 0.009 | 79 |
| 155 | 0.004 | 80 | 0.017 | 82 |
| 156 | 0.005 | 104 | 0.010 | 105 |
| 157 | 0.003 | 97 | 0.004 | 96 |
| 158 | 0.001 | 104 | 0.007 | 107 |
| 159 | 0.002 | 101 | 0.003 | 106 |
| 160 | 0.001 | 104 | 0.004 | 105 |
| 161 | 0.007 | 95 | 0.020 | 98 |
| 162 | 0.001 | 106 | 0.001 | 110 |
| 163 | 0.001 | 107 | 0.002 | 113 |
| 164 | 0.002 | 108 | 0.002 | 114 |
| 165 | 0.002 | 106 | 0.002 | 115 |
| 166 | 0.004 | 85 | 0.037 | 92 |
| 167 | 0.001 | 101 | 0.002 | 100 |
| 168 | 0.001 | 87 | 0.002 | 96 |
| 169 | 0.005 | 86 | 0.023 | 94 |
| 170 | 0.001 | 100 | 0.002 | 101 |
| 171 | 0.005 | 96 | 0.036 | 85 |
| 172 | 0.001 | 94 | 0.004 | 94 |
| 173 | 0.001 | 102 | 0.002 | 109 |
| 174 | 0.003 | 106 | 0.013 | 104 |
| 175 | 0.001 | 107 | 0.001 | 109 |
| 176 | 0.001 | 104 | 0.003 | 102 |
| 177 | 0.002 | 107 | 0.015 | 40 |
| 178 | 0.002 | 106 | 0.002 | 107 |
| 179 | 0.001 | 103 | 0.001 | 108 |
| 180 | 0.003 | 94 | 0.002 | 88 |
| 181 | 0.004 | 95 | 0.009 | 95 |

### STATEMENTS

1. A compound of Formula (I):

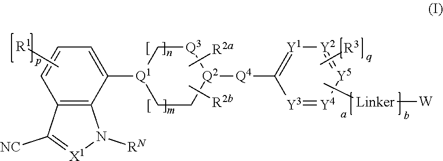

or a pharmaceutically acceptable salt thereof,
wherein:
X¹ is CH or N;
p is 0, 1 or 2;
where:
each R¹ is a substituent on any C atom and is independently selected from F, Cl, C₁₋₃alkyl and C₁₋₃alkoxy, wherein said C₁₋₃alkyl and C₁₋₃alkoxy may be independently optionally substituted by one or more F;
R^{N} is selected from H and Me;
n is 0, 1 or 2;
m is 0 or 1;
Q¹ is CH or N;
when n & m are both other than 0, Q² is CH or N;
when n & m are both 0, Q² is CH;
when n is 0 or 1, Q³ is CH;
when n is 2 and Q² is N, Q³ is CH;
when n is 2 and Q² is CH, Q³ is CH or O;
R^{2a} and R^{2b} are substituents on the same or different C atoms other than at Q¹ or Q², each independently selected from H, F and C₁₋₃alkyl, or R^{2a} & R^{2b} together form a —(CH₂)ᵣ— group where r is 1, 2 or 3;
Q⁴ is a single bond or —NR⁴C(═O);
R⁴ is H or Me;
0, 1 or 2 of Y¹, Y², Y³, Y⁴ & Y⁵ is/are N, and are otherwise C;
each R³ is a substituent on any C atom at Y¹, Y², Y³, Y⁴ & Y₅, and is independently selected from F, Cl, CN, C₁₋₃alkyl and C₁₋₃alkoxy, wherein said C₁₋₃alkyl and C₁₋₃alkoxy may be independently optionally substituted by one or more F;
q is 0, 1 or 2;
wherein Linker is attached at any available C atom at Y⁴ & Y⁵;
Linker is a saturated or a partially or fully unsaturated framework comprising C and H atoms and at least one heteroatom, wherein said framework has end points of attachment ‘a’ and ‘b’ and a length of from 5 to 26 atoms between ‘a’ and ‘b’; wherein said framework may include one or more straight and/or branched chains and/or rings and is optionally substituted on any available C atom(s) by one or more F; and
W is an E3 ubiquitin ligase cereblon binder unit.

2. The compound of Formula (I), or pharmaceutically acceptable salt thereof, according to statement 1 wherein the Linker is a saturated or partially unsaturated framework.

3. The compound of Formula (I), or pharmaceutically acceptable salt thereof, according to statement 1 or statement 2 wherein the Linker comprises C and H atoms and at least two heteroatoms.

4. The compound of Formula (I), or pharmaceutically acceptable salt thereof, according to statement 1 or statement 2 wherein the Linker comprises C and H atoms and at least one nitrogen heteroatom in the form of a secondary or tertiary amine.

5. The compound of Formula (I), or pharmaceutically acceptable salt thereof, according to statement 1 or statement 2 wherein the Linker includes at least one nitrogen-containing saturated or partially unsaturated heterocyclic group having from 4 to 12 ring atoms, or a A¹-CH₂—CH₂-A² unit where A¹ and A² are each independently selected from N and 0.

6. The compound of Formula (I), or pharmaceutically acceptable salt thereof, according to any one of statements 1 to 5 wherein the Linker has the Formula:

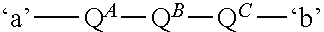

wherein:
‘a’ and ‘b’ represent the end points of attachment;
Q^{A} is -G-Q^{B}- or -G-(C₁₋₅alkylene)-;
Q^{B} is a directbond, -Q^{B1}-Q^{B2}-Q^{B3}- or C₁₋₃alkylene optionally substituted by one or more F (e.g. 1 or 2);
where:
   Q^{B1} & Q^{B3} each independently represent a direct bond or C₁₋₂alkylene;
   Q^{B2} is Q^{H}, —O—CH₂CH₂—O—, —O— or —N(R^{J})— where R^{J} is H or C₁₋₃alkyl;
Q^{C} is -Q^{H}-G- or —(C₁₋₅alkylene)-G-;
each G is independently a direct bond, —CH₂—, —O—, —C(═O)— or —N(R^{G})— where R^{G} is H or C₁₋₃alkyl;
each Q^{H} is independently a 4-12-membered nitrogen-containing saturated or partially unsaturated heterocyclic group;
where the values of Q^{A}, Q^{B} and Q^{C} are selected so that the Linker does not contain any N—N or N—O bonds.

7. The compound of Formula (I), or pharmaceutically acceptable salt thereof, according to statement 6 wherein Q^{B} is a direct bond, —CH₂— or —CH₂CH₂—.

8. The compound of Formula (I), or pharmaceutically acceptable salt thereof, according to statement 6 or statement 7 wherein Q^{C} is -Q^{H}-G- or —(C₁₋₂alkylene)-G-.

9. The compound of Formula (I), or pharmaceutically acceptable salt thereof, according to any one of statements 6 to 8, wherein each Q^{H} is independently selected from piperazin-1,4-diyl, azetidin-1,3-diyl, piperidin-1,4-diyl, 1,4-diazepan-1,4-diyl, 12-oxa-3,9-diazaspiro[5.6]dodecan-3,9-diyl, pyrrolidin-1,3-diyl, 3,9-diazaspiro[5.5]undecan-3,9-diyl, 2,5-diazabicyclo[2.2.1]heptan-2,5-diyl, 1,3,3a,4,6,6a-hexahydropyrrolo[3,4-c]pyrrol-2,5-diyl, 3,6-dihydro-2H-pyridin-1,4-diyl, morpholin-2,3-diyl, 2-azaspiro[3.5]nonan-2,7-diyl and 9-azaspiro[5.5]undecan-3,9-diyl.

10. The compound of Formula (I), or pharmaceutically acceptable salt thereof, according to any one of statements 1 to 9, wherein the Linker is selected from any of Linkers 1 to 58 as shown in the description.

11. A PROTAC compound or a pharmaceutically acceptable salt thereof, containing an E3 ubiquitin ligase cereblon binder unit and an AR binding unit of Formula (Ia):

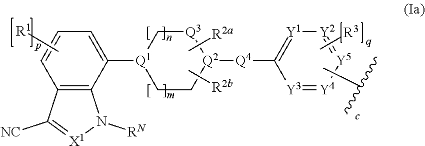

where R¹, p, X¹, R^{N}, n, m, R^{2a}, R^{2b}, Q¹, Q², Q³, Q⁴, Y¹, Y², Y³, Y⁴, Y⁵, R³ and q are as defined in statement 1.

12. The PROTAC compound or a pharmaceutically acceptable salt thereof, according to statement 11, containing an E3 ubiquitin ligase cereblon binder unit and an AR binding unit of Formula (Ia) of Formula (Ib):

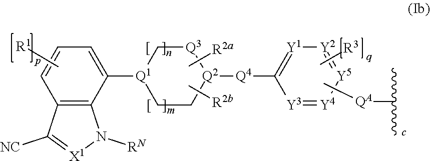

where:
Q^{A} is -G-Q^{H}- or -G-(C₁₋₅alkylene)-;
G is a direct bond, —CH₂—, —O—, —C(═O)— or —N(R^{G})— where R^{G} is H or C₁₋₃alkyl; and
Q^{H} is a 4-12-membered nitrogen-containing saturated or partially unsaturated heterocyclic group.

13. The PROTAC compound or a pharmaceutically acceptable salt thereof, according to statement 12, containing an E3 ubiquitin ligase cereblon binder unit and an AR binding unit of Formula (Ia) of Formula (Ic):

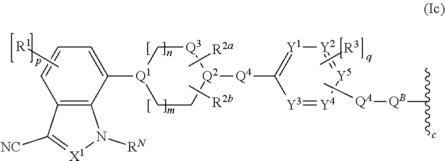

where:
Q^{B} is a direct bond, -Q^{B1}-Q^{B2}-Q^{B3}- or C₁₋₃alkylene optionally substituted by one or more F (e.g. 1 or 2);
where:
   Q^{B1} & Q^{B3} each independently represent a direct bond or C₁₋₂alkylene;
   Q^{B2} is Q^{H}, —O—CH₂CH₂—O—, —O— or —N(R^{J})— where R^{J} is H or C₁₋₃alkyl;
and each Q^{H} is independently a 4-12-membered nitrogen-containing saturated or partially unsaturated heterocyclic group;
where the values of Q^{A} and Q^{B} are selected so that Formula (Ic) does not contain any N—N or N—O bonds.

14. The PROTAC compound or a pharmaceutically acceptable salt thereof, according to statement 12 or statement 13, where:
Q^{A} is -G^{A}-Q^{HA}- or -G^{A}-(C₁₋₅alkylene)- where:
G^{A} is selected from a direct bond, —CH₂—, —C(═O)—, —O— or —N(R^{G})—;
R^{G} is H or C₁₋₃alkyl; and
Q^{HA} is a 4 to 11-membered nitrogen-containing saturated heterocyclic group.

15. A compound of Formula (II):

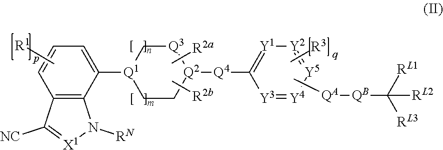

or a salt thereof, wherein:
Q^{A} is -G-Q^{H}- or -G-(C₁₋₅alkylene)-;
G is a direct bond, —CH₂—, —C(═O)—, —O—, or —N(R^{G})— where R^{G} is H or C₁₋₃alkyl;
Q^{H} is a 4-12-membered nitrogen-containing saturated or partially unsaturated heterocyclic group;
Q^{D} is a direct bond or C₁₋₂alkylene optionally substituted by one or more F (for example 1 or 2 F); and
   (i) R^{L1} & R^{L2} together form “═O” and R^{L3} is H or C₁₋₆alkoxy;
   (ii) R^{L1} & R^{L2} are each independently C₁₋₆alkoxy, and R^{L3} is H;
   (iii) R^{L1} & R^{L2} together form —O—(CH₂)ₖ—O— where k is 2 or 3, and R^{L3} is H; or
   (iv) R^{L1} is OH or LG¹ where LG¹ is a leaving group, and R^{L2} & R^{L3} are both H;
where when Q^{D} is a direct bond and Q^{A} is -G-Q^{H}-, the value of Q^{H} is selected so that Q^{D} connects to a C atom of Q^{H}.
and R¹, p, X¹, R^{N}, n, M, R^{a2}, R^{b2}, Q¹, Q², Q³, Q⁴, Y¹, Y², Y³, Y⁴, Y⁵, R³ and q are as defined in statement 1.

16. The compound of Formula (II) or a salt thereof, according to statement 15, wherein LG¹ is selected from Cl, Br, I, trifluoromethanesulfonate and C₁₋₇hydrocarbylsulfonate.

17. A compound of Formula (IV):

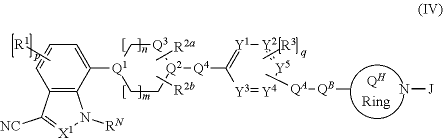

or a salt thereof, wherein:
J is H or PG² where PG² is a nitrogen protecting group (for example a tert-butoxycarbonyl group);
Q^{A} is -G-Q^{H}- or -G-(C₁₋₅alkylene)-;
G is a direct bond, —CH₂—, —C(═O)—, —O—, or —N(R^{G})— where R^{G} is H or C₁₋₃alkyl;
Q^{B} is a direct bond, -Q^{B1}-Q^{B2}-Q^{B3}- or C₁₋₃alkylene optionally substituted by one or more F (e.g. 1 or 2);
where:
   Q^{B1} & Q^{B3} each independently represent a direct bond or C₁₋₂alkylene;
   Q^{B2} is Q^{H}, —O—CH₂CH₂—O—, —O— or —N(R^{J})— where R^{J} is H or C₁₋₃alkyl;
and each Q^{H} (including the “Q^{H} Ring” attached to J) is independently a 4-12-membered nitrogen-containing saturated or partially unsaturated heterocyclic group;
where the values of Q^{A}, Q^{B} and Q^{H} Ring are selected so that Formula (TV) does not contain any N—N or N—O bonds; and R¹, p, X¹, R^{N}, m, R^{2a}, R^{2b}, Q¹, Q², Q³, Q⁴, Y¹, Y², Y³, Y⁴, Y⁵, R³ and q are as defined in statement 1.

18. The compound of Formula (IV), or a salt thereof, as statemented in statement 17, wherein PG² is C₁₋₆alkoxycarbonyl.

19. A compound of Formula (V):

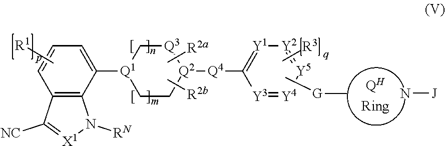

or a salt thereof, wherein:
X^{X} is selected from:
   (i) N substituted by J where J is H or PG³ where PG³ is a protecting group; and
   (ii) C substituted by oxo, or by R^{U1} and R^{U2}; where R^{U1} and R^{U2} are each C₁₋₆alkoxy;
or R^{U1} and R^{U2} together represent —O—(CH₂)ᵤ—O— where u is 2 or 3;
G is a direct bond, —CH₂—, —C(═O)—, —O—, or —N(R^{G})— where R^{G} is H or C₁₋₃alkyl;
Q^{H} Ring is a 4-12-membered nitrogen-containing saturated or partially unsaturated heterocyclic group;
where the values of G and Q^{H} Ring are selected so that Formula (V) does not contain any N—N or N—O bonds; and R¹, p, X¹, R^{N}, n, m, R^{2a}, R^{2b}, Q¹, Q², Q³, Q⁴, Y¹, Y², Y³, Y⁴, Y⁵, R³ and q are as defined in statement 1.

20. The compound of Formula (V), or a salt thereof, according to statement 19, wherein PG³ is C₁₋₆alkoxycarbonyl.

21. The compound of Formula (I), or a pharmaceutically acceptable salt thereof, according to any one of statements 1 to 10, wherein W is W1 which is: —Z—(R^{A})ₕ
where Z is:

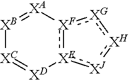

wherein:
[symbol] represents a single covalent bond or a double covalent bond;
0, 1 or 2 of X^{A}, X^{B}, X^{C}, X^{D}, X^{E} & X^{F} is/are N, where X^{E} & X^{F} are not both N, and are otherwise C;
1 of X^{G}, X^{H} & X^{J} is C(O);
1 of X^{G}, X^{H} & X^{J} is N-(2,6-dioxopiperidin-3-yl) (Y); and
1 of X^{G}, X^{H} & X^{J} is selected from C(RT)₂, —CH₂CH₂—, C(O), N(C₁₋₃ alkyl), —O— and —N═, wherein each R^{T} is selected from H, F, Me or together with the carbon of C(R^{T})₂ forms a cycloprop-1,1-diyl group;
   where X^{G}, X^{H} & X^{J} are selected such that there are not two C(O) groups present at adjacent positions, and that the N-(2,6-dioxopiperidin-3-yl) is not at an adjacent position to either N(C₁₋₃alkyl) or O;
each R^{A} is independently a substituent on any available C atom at X^{A}, X^{B}, X^{C} or X^{D} selected from F, Cl, C₁₋₃alkyl, C₁₋₃alkoxy wherein said C₁₋₃alkyl and C₁₋₃alkoxy is independently optionally substituted by one or more F;
h is 0, 1 or 2; and
wherein the Linker is attached to any C atom at X^{A}, X^{B}, X^{C} or X^{D}.

22. The compound of Formula (I), or a pharmaceutically acceptable salt thereof, according to statement 21 where each R^{A} is a substituent on any available C at X^{A}, X^{B}, X^{c} or X^{D} selected from F, Cl and C₁₋₃alkoxy optionally substituted by one or more F.

23. The compound of Formula (I), or a pharmaceutically acceptable salt thereof, according to statement 21 or statement 22 where X^{G}—X^{H}—X^{J} is:
(i) X^{G}—NY—C(O) where X^{G} is —CH₂—, —CH₂CH₂—, ═N— or C(O);
(ii) X^{G}—C(O)—NY where X^{G} is —O— or N(C₁₋₃alkyl); or
(iii) C(O)—NY—CH₂.

24. The compound of Formula (I), or a pharmaceutically acceptable salt thereof, according to any one of statements 21 to 23 where —Z—(R^{A})ₕ together represent any of the groups 1 to 21 or 27 as shown in the description.

25. The compound of Formula (I), or a pharmaceutically acceptable salt thereof, according to any one of statements 1 to 10, wherein W is W2 which is

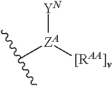

Z^{A} is:

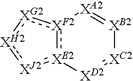

wherein:
[symbol] represents a single covalent bond or a double covalent bond;
1 of X^{A2}, X^{B2}, X^{C2} & X^{D2} is C and covalently bound to Y^{N};
0, 1 or 2 of X^{A2}, X^{B2}, X^{C2}, X^{D2}, X^{E2} & X^{F2} is/are N, where X^{E2} & X^{F2} are not both N, and are otherwise C;
   1 or 2 of X^{G2}, X^{H2} & X^{J2} is/are N; and are otherwise C;
each R^{AA} is a substituent on any available C or N atom of Z—in each case independently selected from R^{AA1} optionally substituted by one or more R^{AA2}; where R^{AA} is further selected from R^{AA2} when R^{AA} is a substituent on an available C atom of Z^{A};
each R^{AA1} is independently C₁₋₄alkyl, C₂₋₃alkenyl, C₂₋₃alkynyl, C₁₋₃alkoxyC₁₋₃alkyl, carboxyC₁₋₃alkyl, C₅₋₇carbocyclyl or a 4-6 membered heterocyclyl;
each R^{AA2} is independently selected from F, Cl, Br, CN, NH₂, C₁₋₃alkyl, O(C₁₋₃alkyl), NH(C₁₋₃alkyl) and N(C₁₋₃alkyl)₂; wherein said C₁₋₃alkyls are optionally substituted by one or more F;
v is 0, 1 or 2;
Y^{N} is 2,4-dioxohexahydropyrimidin-1-yl.

26. The compound or salt thereof according to statement 25, wherein
1 of X^{A2} & X^{B2} is C and covalently bound to Y and the other of X^{A2} & X^{B2} is C;
0 or 1 of X^{C2} & X^{D2} is N and is/are otherwise C;
1 of X^{G2} & X^{J2} is N and the other of X^{G2} & X_{J2} is C; and
X^{H2}, X^{E2} & X^{F2} are all C.

27. The compound or salt thereof according to statement 25, wherein W is W2-1

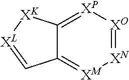

wherein:
X^{K} and X^{L} are either N-linker and CH, N-linker and CMe or NMe and C-linker respectively;
1 of X^{M} and X^{O} is C-2,4-dioxohexahydropyrimidin-1-yl (Y^{N});
0 or 1 of X^{M} and X^{N} is C—F;
X^{N} may be N if X^{M} is not C—F;
the remainder of X^{N}, X^{M} and X^{O} are CH;
X^{P} is CH or CMe.

28. The compound or salt thereof according to statement 27, wherein W represents any of the groups 22 to 26 or 29 or 31 shown below:

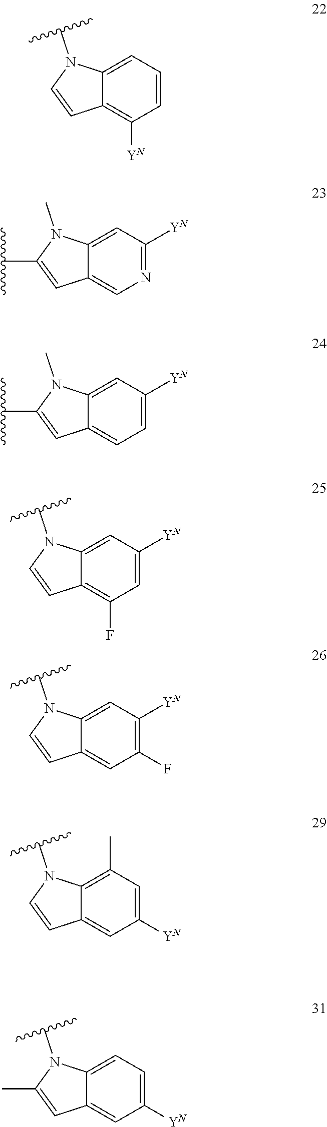

29. The compound of Formula (I), or a pharmaceutically acceptable salt thereof, according to any one of statements 1 to 10, wherein W is W3 which is: —Z^{B}
where Z^{B} is:

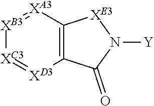

X^{E3} is selected from C(═O) and CR^{E1}R^{E2}, where R^{E1} and R^{E2} are independendently selected from the group consisting of H and C₁₋₃ alkyl, or R^{E1} and R^{E2} taken together with the carbons to be they are attached form a C₃₋₆ cycloalkane ring;
Y is N-(2,6-dioxopiperidin-3-yl);
All of X^{A3}, X^{B3}, X^{C3} and X^{D3} are C;
one pair of X^{A3} and X^{B3}, X^{B3} and X^{C3} and X^{C3} and X^{D3} form a five or six membered fully unsaturated or partially unsaturated N containing heterocylic ring, which is attached to the ‘b’ end of the Linker via a N atom;
the others of X^{A3}, X^{B3}, X^{C3} and X^{D3} bear a group RB, where each RB is selected from H, F, Cl, C₁₋₃ alkyl and C₁₋₃ alkoxy.

30. The compound or salt thereof according to statement 29, wherein X^{B3} and X^{C3} form a five membered partially unsaturated N containing heterocylic ring, which is attached to the ‘b’ end of the Linker via a N atom.

31. The compound or salt thereof according to either statement 29 or statement 30, wherein W represents group 28 shown below:

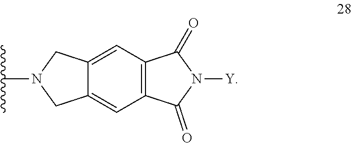

32. The compound of Formula (I), or a pharmaceutically acceptable salt thereof, according to any one of statements 1 to 10, wherein W is W3 which is: —Z^{C}
where Z^{C} is:

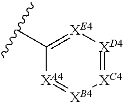

one of X^{B4} and X^{C4} is C—C(═O)—NH—Y;
one of X^{A4}, X^{B4}, X^{C4}, X^{D4} and X^{E4} may be C—F; or
one or two of X^{A4}, X^{B4}, X^{C4}, X^{D4} and X^{E4} may be N;
the remainder of X^{A4}, X^{B4}, X^{C4}, X^{D4} and X^{E4} are CH.

33. The compound or salt thereof according to statement 31, wherein X^{C4} is C—C(═O)—NH—Y and none of X^{A4}, X^{B4}, X^{C4}, X^{D4} and X^{E4} are N.

34. The compound or salt thereof according to either statement 31 or statement 32, wherein W represents group 30 shown below:

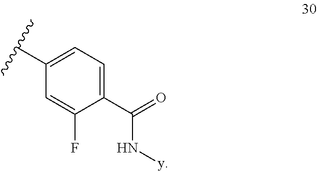

35. The compound or salt thereof, according to any preceding statement wherein R^{N} is H, and p is 0 or 1, R¹ is selected from Cl, F and Me, and R¹, if present, is bound para to Q¹.

36. The compound or salt thereof, according to any preceding statement wherein X¹ is CH.

37. The compound or salt thereof, according to any preceding statement wherein X¹ is N.

38. The compound or salt thereof, according to any preceding statement wherein:
(a) n is 0 and m is 1; or
(b) n is 1 and m is 1; or
(c) n is 2 and m is 0.

39. The compound or salt thereof, according to any preceding statement wherein Q¹ is N, Q² is CH and Q³ is CH.

40. The compound or salt thereof, according to any one of statements 1 to 37 wherein Q¹ is N, Q² is CH and Q³ is CH, and n and m are 1 and 1 or 2 and 0 respectively.

41. The compound or salt thereof, according to any preceding statement wherein R^{2a} and R^{2b} are both H.

42. The compound or salt thereof, according to any preceding statement wherein:
(a) Q⁴ is a single bond; or
(b) Q⁴ is a NR⁴C(═O) and R⁴ is H.

43. The compound or salt thereof according to any preceding statement wherein Y¹, Y², Y³, Y⁴ & Y⁵ are respectively selected from (C, C, C, C, C), (N, C, C, C, C), (C, N, C, C, C), (N, C, C, N, C), (N, N, C, C, C), (N, C, N, C, C), (C, N, C, N, C) and (C, N, N, C, C).

44. The compound or salt thereof according to any preceding statement wherein Y¹, Y², Y³, Y⁴ & Y⁵ are respectively selected from (C, C, C, C, C), (N, C, C, C, C) and (N, N, C, C, C).

45. The compound or salt thereof according to any preceding statement wherein Y¹, Y², Y³, Y⁴ & Y⁵ are all C.

46. The compound or salt thereof according to any preceding statement wherein q is 0, 1 or 2 and R³ (when present) is a substituent on any C atom at Y¹, Y², Y³, Y⁴ & Y⁵ selected from F, CN and methyl.

47. The compound or salt thereof according to any preceding statement wherein q is 0 or 1 and R³ (when present) is a substituent on any C atom at Y¹, Y², Y³, Y⁴ & Y⁵ which is F.

48. The compound or salt thereof according to any preceding statement wherein Linker is attached at Y⁵.

49. The compound or salt thereof according to any one of statements 1 to 34, wherein:
X¹ is CH;
p is 1;
R^{N} is H;
n is 1;
m is 1;
R^{2a} and R^{2b} are both H;
Q² is CH; Q³ is CH;
Q⁴ is a single bond;
Y¹, Y², Y³ and Y⁴ are all CH;
Y⁵ is C and has the Linker attached;
q is 0.

50. The compound or salt thereof according to any one of statements 1 to 34, wherein:
X¹ is N;
p is 1;
R^{N} is H;
n is 2;
m is 0;
R^{2a} and R^{2b} are both H;
Q⁴ is a single bond;
Y¹, Y², Y³ and Y⁴ are all CH;
Y⁵ is C and has the Linker attached;
q is 0.

51. The compound or salt thereof according to any one of statements 1 to 34, wherein:
X¹ is CH;
p is 1;
R^{N} is H;
n is 1;
m is 0;
R^{2a} and R^{2b} are both H;
Q⁴ is a single bond;
Y¹, Y², Y³ and Y⁴ are all CH;
Y⁵ is C and has the Linker attached;
q is 0.

52. A pharmaceutical composition, which comprises a compound of Formula (I) or PROTAC of Formula (Ia), or a pharmaceutically acceptable salt thereof according to one of statements 1-14 or 21-51, in association with a pharmaceutically acceptable excipient.

53. A pharmaceutical composition, which comprises a compound of Formula (I) or PROTAC of Formula (Ia), or a pharmaceutically acceptable salt thereof, according to any one of statements 1-14 or 21-51, for use in the treatment of cancer.

54. A pharmaceutical composition, which comprises a compound of Formula (I) or PROTAC of Formula (Ia), or a pharmaceutically acceptable salt thereof, according to any one of statements 1-14 or 21-51, for use in the treatment of a solid tumour.

55. A pharmaceutical composition, which comprises a compound of Formula (I) or PROTAC of Formula (Ia), or a pharmaceutically acceptable salt thereof, according to any one of statements 1-14 or 21-51, for use in the treatment of an AR-sensitive tumour type.

56. A pharmaceutical composition, which comprises a compound of Formula (I) or PROTAC of Formula (Ia), or a pharmaceutically acceptable salt thereof, according to any one of statements 1-14 or 21-51, for use in the treatment of tumour types that harbour one or more mutated forms of the androgen receptor.

57. A pharmaceutical composition, which comprises a compound of Formula (I) or PROTAC of Formula (Ia), or a pharmaceutically acceptable salt thereof, according to any one of statements 1-14 or 21-51, for use in the treatment of prostate cancer.

58. A pharmaceutical composition, which comprises a compound of Formula (I) or PROTAC of Formula (Ia), or a pharmaceutically acceptable salt thereof, according to any one of statements 1-14 or 21-51, for use in the treatment of castrate-resistant prostate cancer.

59. A pharmaceutical composition, which comprises a compound of Formula (I) or PROTAC of Formula (Ia), or a pharmaceutically acceptable salt thereof, according to any one of statements 1-14 or 21-51, for use in the treatment of metastatic castrate-resistant prostate cancer.

60. A pharmaceutical composition, which comprises a compound of Formula (I) or PROTAC of Formula (Ia), or a pharmaceutically acceptable salt thereof, according to any one of statements 1-14 or 21-51, for use in the treatment of AR-mutated cancer.

61. The compound of Formula (I) or PROTAC of Formula (Ia), or a pharmaceutically acceptable salt thereof, according to any one of statements 1-14 or 21-51, for use as a medicament.

62. The compound of Formula (I) or PROTAC of Formula (Ia), or a pharmaceutically acceptable salt thereof, according to any one of statements 1-14 or 21-51, for use in therapy.

63. The compound of Formula (I) or PROTAC of Formula (Ia), or a pharmaceutically acceptable salt thereof, according to any one of statements 1-14 or 21-51, for use in a method of treatment of the human or animal body by therapy.

64. The compound of Formula (I) or PROTAC of Formula (Ia), or a pharmaceutically acceptable salt thereof, according to any one of statements 1-14 or 21-51, for use in the production of an anti-proliferative effect in a warm-blooded animal such as man.

65. Use of a compound of Formula (I) or PROTAC of Formula (Ia), or a pharmaceutically acceptable salt thereof, according to any one of statements 1-14 or 21-51, for the manufacture of a medicament for the production of an anti-proliferative effect in a warm-blooded animal such as man.

66. A method for producing an anti-proliferative effect in a warm-blooded animal, such as man, in need of such effect, which comprises administering to said animal an effective amount of a compound of Formula (I) or PROTAC of Formula (Ia), or a pharmaceutically acceptable salt thereof, according to any one of statements 1-14 or 21-51.

67. A compound of Formula (I) or PROTAC of Formula (Ia), or a pharmaceutically acceptable salt thereof, according to any one of statements 1-14 or 21-51, for use as an anti-invasive agent in the containment and/or treatment of solid tumour disease.

68. Use of a compound of Formula (I) or PROTAC of Formula (Ia), or a pharmaceutically acceptable salt thereof, according to any one of statements 1-14 or 21-51, for the manufacture of a medicament for use as an anti-invasive agent in the containment and/or treatment of solid tumour disease.

69. A method for producing an anti-invasive effect by the containment and/or treatment of solid tumour disease, in a warm-blooded animal, such as man, in need of such effect, which comprises administering to said animal an effective amount of a compound of Formula (I) or PROTAC of Formula (Ia), or a pharmaceutically acceptable salt thereof, according to any one of statements 1-14 or 21-51.

70. A compound of Formula (I) or PROTAC of Formula (Ia), or a pharmaceutically acceptable salt thereof, according to any one of statements 1-14 or 21-51, for use in the prevention or treatment of cancer.

71. Use of a compound of Formula (I) or PROTAC of Formula (Ia), or a pharmaceutically acceptable salt thereof, according to any one of statements 1-14 or 21-51, for the manufacture of a medicament for the prevention or treatment of cancer.

72. A method for the prevention or treatment of cancer in a warm-blooded animal, such as man, in need of such treatment, which comprises administering to said animal an effective amount of a compound of Formula (I) or PROTAC of Formula (Ia), or a pharmaceutically acceptable salt thereof, according to any one of statements 1-14 or 21-51.

73. A compound of Formula (I) or PROTAC of Formula (Ia), or a pharmaceutically acceptable salt thereof, according to any one of statements 1-14 or 21-51, for use in the prevention or treatment of solid tumour(s).

74. Use of a compound of Formula (I) or PROTAC of Formula (Ia), or a pharmaceutically acceptable salt thereof, according to any one of statements 1-14 or 21-51 for the manufacture of a medicament for the prevention or treatment of solid tumour(s).

75. A method for the prevention or treatment of solid tumour(s) in a warm-blooded animal, such as man, in need of such treatment, which comprises administering to said animal an effective amount of a compound of Formula (I) or PROTAC of Formula (Ia), or a pharmaceutically acceptable salt thereof, according to any one of statements 1-14 or 21-51.

76. A compound of Formula (I) or PROTAC of Formula (Ia), or a pharmaceutically acceptable salt thereof, according to any one of statements 1-14 or 21-51, for use in the prevention or treatment of tumour types that are sensitive to degradation of androgen receptors.

77. Use of a compound of Formula (I) or PROTAC of Formula (Ia), or a pharmaceutically acceptable salt thereof, according to any one of statements 1-14 or 21-51, for the manufacture of a medicament for the prevention or treatment of those tumour types that are sensitive to degradation of androgen receptors.

78. A method for the prevention or treatment of those tumour types that are sensitive to degradation of androgen receptors in a warm-blooded animal, such as man, in need of such treatment, which comprises administering to said animal an effective amount of a compound of Formula (I) or PROTAC of Formula (Ia), or a pharmaceutically acceptable salt thereof, according to any one of statements 1-14 or 21-5151.

79. A compound of Formula (I) or PROTAC of Formula (Ia), or a pharmaceutically acceptable salt thereof, according to any one of statements 1-14 or 21-51, for use in providing a degrading effect on androgen receptors in a warm-blooded animal such as man.

80. Use of a compound of Formula (I) or PROTAC of Formula (Ia), or a pharmaceutically acceptable salt thereof, according to any one of statements 1-14 or 21-51, for the manufacture of a medicament for providing a degrading effect on androgen receptors in a warm-blooded animal such as man.

81. A method for providing a degrading effect on androgen receptors in a warm-blooded animal, such as man, in need of such effect, which comprises administering to said animal an effective amount of a compound of Formula (I) or PROTAC of Formula (Ia), or a pharmaceutically acceptable salt thereof, according to any one of statements 1-14 or 21-51.

82. A compound of Formula (I) or PROTAC of Formula (Ia), or a pharmaceutically acceptable salt thereof, according to any one of statements 1-14 or 21-51, for use in providing a selective degrading effect on androgen receptors in a warm-blooded animal such as man.

83. Use of a compound of Formula (I) or PROTAC of Formula (Ia), or a pharmaceutically acceptable salt thereof, according to any one of statements 1-14 or 21-51, for the manufacture of a medicament for providing a selective degrading effect on androgen receptors in a warm-blooded animal such as man.

84. A method for providing a selective degrading effect on androgen receptors in a warm-blooded animal, such as man, in need of such effect, which comprises administering an effective amount of a compound of Formula (I) or PROTAC of Formula (Ia), or a pharmaceutically acceptable salt thereof, according to any one of statements 1-14 or 21-51.

85. A compound of Formula (I) or PROTAC of Formula (Ia), or a pharmaceutically acceptable salt thereof, according to any one of statements 1-14 or 21-51, for use in the treatment of tumour types that harbour androgen receptor mutations.

86. Use of a compound of Formula (I) or PROTAC of Formula (Ia), or a pharmaceutically acceptable salt thereof, according to any one of statements 1-14 or 21-51, for the manufacture of a medicament for the prevention or treatment of those tumour types that harbour androgen receptor mutations.

87. A method for the prevention or treatment of those tumour types that harbour androgen receptor mutations in a warm-blooded animal, such as man, in need of such prevention or treatment, which comprises administering to said animal an effective amount of a compound of Formula (I) or PROTAC of Formula (Ia), or a pharmaceutically acceptable salt thereof, according to any one of statements 1-14 or 21-51.

88. A compound of Formula (I) or PROTAC of Formula (Ia), or a pharmaceutically acceptable salt thereof, according to any one of statements 1-14 or 21-51, for use in the treatment of prostate cancer (for example castrate-resistant prostate cancer (CRPC), for example metastatic CRPC)).

89. Use of a compound of Formula (I) or PROTAC of Formula (Ia), or a pharmaceutically acceptable salt thereof, according to any one of statements 1-14 or 21-51, for the manufacture of a medicament for the treatment of prostate cancer (for example castrate-resistant prostate cancer (CRPC), for example metastatic CRPC).

90. A method for treating prostate cancer (for example castrate-resistant prostate cancer (CRPC), for example metastatic CRPC) in a warm-blooded animal, such as man, in need of such treatment, which comprises administering to said animal an effective amount of a compound of Formula (I) or PROTAC of Formula (Ia), or a pharmaceutically acceptable salt thereof, according to any one of statements 1-14 or 21-51.

1. A compound of Formula (I): 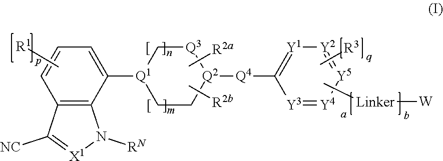
or a pharmaceutically acceptable salt thereof,
wherein:
X¹ is CH or N;
p is 0, 1 or 2;
where:
each R¹ is a substituent on any C atom and is independently selected from F, Cl, C₁₋₃alkyl and C₁₋₃alkoxy, wherein said C₁₋₃alkyl and C₁₋₃alkoxy may be independently optionally substituted by one or more F;
R^{N} is selected from H and Me;
n is 0, 1 or 2;
m is 0 or 1;
Q¹ is CH or N;
when n & m are both other than 0, Q² is CH or N;
when n & m are both 0, Q² is CH;
when n is 0 or 1, Q³ is CH;
when n is 2 and Q² is N, Q³ is CH;
when n is 2 and Q² is CH, Q³ is CH or O;
R^{2a} and R^{2b} are substituents on the same or different C atoms other than at Q¹ or Q², each independently selected from H, F and C₁₋₃alkyl, or R^{2a} & R^{2b} together form a —(CH₂)ᵣ— group where r is 1, 2 or 3;
Q⁴ is a single bond or —NR⁴C(═O);
R⁴ is H or Me;
0, 1 or 2 of Y¹, Y², Y³, Y4 & Y⁵ is/are N, and are otherwise C;
each R³ is a substituent on any C atom at Y¹, Y², Y³, Y4 & Y⁵, and is independently selected from F, Cl, CN, C₁₋₃alkyl and C₁₋₃alkoxy, wherein said C₁₋₃alkyl and C₁₋₃alkoxy may be independently optionally substituted by one or more F;
q is 0, 1 or 2;
wherein Linker is attached at any available C atom at Y⁴ & Y⁵;
Linker is a saturated or a partially or fully unsaturated framework comprising C and H atoms and at least one heteroatom, wherein said framework has end points of attachment ‘a’ and ‘b’ and a length of from 5 to 26 atoms between ‘a’ and ‘b’;
wherein said framework may include one or more straight and/or branched chains and/or rings and is optionally substituted on any available C atom(s) by one or more F; and
W is an E3 ubiquitin ligase cereblon binder unit.

2. The compound of Formula (I), or pharmaceutically acceptable salt thereof, according to claim 1 or claim 2 wherein the Linker is a saturated or partially unsaturated framework and:
(a) comprises C and H atoms and at least two heteroatoms; or
(b) comprises C and H atoms and at least one nitrogen heteroatom in the form of a secondary or tertiary amine; or
(c) includes at least one nitrogen-containing saturated or partially unsaturated heterocyclic group having from 4 to 12 ring atoms, or a A¹-CH₂—CH₂-A² unit where A¹ and A² are each independently selected from N and O.

3. The compound of Formula (I), or pharmaceutically acceptable salt thereof, according to either claim 1 or claim 2 wherein the Linker has the Formula: 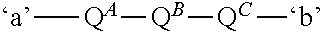
wherein:
‘a’ and ‘b’ represent the end points of attachment;
Q^{A} is -G-Q^{H}- or -G-(C₁₋₅alkylene)-;
Q^{B} is a directbond, -Q^{B1}-Q^{B2}-Q^{B3}- or C₁₋₃alkylene optionally substituted by one or more F (e.g. 1 or 2);
where:
Q^{B1} & Q^{B3} each independently represent a direct bond or C₁₋₂alkylene;
Q^{B2} is Q^{H}, —O—CH₂CH₂—O—, —O— or —N(R^{J})— where R^{J} is H or C₁₋₃alkyl;
Q^{C} is -Q^{H}-G- or —(C₁₋₅alkylene)-G-;
each G is independently a direct bond, —CH₂—, —O—, —C(═O)— or —N(R^{G})— where R^{G} is H or C₁₋₃alkyl;
each Q^{H} is independently a 4-12-membered nitrogen-containing saturated or partially unsaturated heterocyclic group;
where the values of Q^{A}, Q^{B} and QC are selected so that the Linker does not contain any N—N or N—O bonds.

4. The compound of Formula (I), or pharmaceutically acceptable salt thereof, according to claim 3 wherein:
(a) Q^{B} is a direct bond, —CH₂— or —CH₂CH₂—; and/or
(b) Q^{C} is -Q^{H}-G- or —(C₁₋₂alkylene)-G-; and/or
(c) each Q^{H} is independently selected from piperazin-1,4-diyl, azetidin-1,3-diyl, piperidin-1,4-diyl, 1,4-diazepan-1,4-diyl, 12-oxa-3,9-diazaspiro[5.6]dodecan-3,9-diyl, pyrrolidin-1,3-diyl, 3,9-diazaspiro[5.5]undecan-3,9-diyl, 2,5-diazabicyclo[2.2.1]heptan-2,5-diyl, 1,3,3a,4,6,6a-hexahydropyrrolo[3,4-c]pyrrol-2,5-diyl, 3,6-dihydro-2H-pyridin-1,4-diyl, morpholin-2,3-diyl, 2-azaspiro[3.5]nonan-2,7-diyl and 9-azaspiro[5.5]undecan-3,9-diyl.

5. A PROTAC compound or a pharmaceutically acceptable salt thereof, containing an E3 ubiquitin ligase cereblon binder unit and an AR binding unit of Formula (Ia): 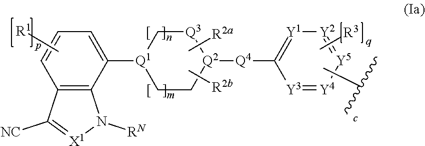
where R¹, p, X¹, R^{N}, n, m, R^{2a}, R^{2b}, Q¹, Q², Q³, Q⁴, Y¹, Y², Y³, Y⁴, Y⁵, R³ and q are as defined in claim 1.

6. The compound of Formula (I), or a pharmaceutically acceptable salt thereof, according to any one of claims 1 to 4, wherein W is W2 which is 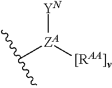
Z^{A} is: 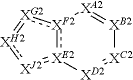
wherein: [symbol]
represents a single covalent bond or a double covalent bond;
1 of X^{A2}, X^{B2}, X^{C2} & X^{D2} is C and covalently bound to Y^{N};
0, 1 or 2 of X^{A2}, X^{B2}, X^{C2}, X^{D2}, X^{E2} & X^{F2} is/are N, where X^{E2} & X^{F2} are not both N, and are otherwise C;
1 or 2 of X^{G2}, X^{H2} & X^{J2} is/are N; and are otherwise C;
each R^{AA} is a substituent on any available C or N atom of Z—in each case independently selected from R^{AA1} optionally substituted by one or more R^{AA2}; where R^{AA} is further selected from R^{AA2} when R^{AA} is a substituent on an available C atom of Z^{A};
each R^{AA1} is independently C₁₋₄alkyl, C₂₋₃alkenyl, C₂₋₃alkynyl, C₁₋₃alkoxyC₁₋₃alkyl, carboxyC₁₋₃alkyl, C₅₋₇carbocyclyl or a 4-6 membered heterocyclyl;
each R^{AA2} is independently selected from F, Cl, Br, CN, NH₂, C₁₋₃alkyl, O(C₁₋₃alkyl), NH(C₁₋₃alkyl) and N(C₁₋₃alkyl)₂; wherein said C₁₋₃alkyls are optionally substituted by one or more F;
v is 0, 1 or 2;
Y^{N} is 2,4-dioxohexahydropyrimidin-1-yl.

7. The compound or pharmaceutically acceptable salt thereof according to claim 6, wherein
1 of X^{A2} & X^{B2} is C and covalently bound to Y and the other of X^{A2} & X^{B2} is C;
0 or 1 of X^{C2} & X^{D2} is N and is/are otherwise C;
1 of X^{G2} & X^{J2} is N and the other of X^{G2} & X^{J2} is C; and
X^{H2}, X^{E2} & X^{F2} are all C.

8. The compound or pharmaceutically acceptable salt thereof according to claim 6, wherein W is W2-1 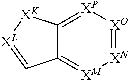
wherein:
X^{K} and X^{L} are either N-linker and CH, N-linker and CMe or NMe and C-linker respectively;
1 of XM and X^{O} is C-2,4-dioxohexahydropyrimidin-1-yl (Y^{N});
0 or 1 of X and X^{N} is C—F;
X^{N} may be N if X^{M} is not C—F;
the remainder of X^{N}, X^{M} and X^{O} are CH; and
X^{P} is CH or CMe.

9. The compound of Formula (I), or a pharmaceutically acceptable salt thereof, according to any one of claims 1 to 4, wherein W is W1 which is: —Z—(R^{A})ₕ
where Z is: 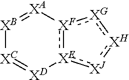
wherein: [symbol]
represents a single covalent bond or a double covalent bond;
0, 1 or 2 of X^{A}, X^{B}, X^{C}, X^{D}, X^{E} & X^{F} is/are N, where X^{E} & X^{F} are not both N, and are otherwise C;
1 of X^{G}, X^{H} & X^{J} is C(O);
1 of X^{G}, X^{H} & X^{J} is N-(2,6-dioxopiperidin-3-yl) (Y); and
1 of X^{G}, X^{H} & X^{J} is selected from C(R^{T})₂, —CH₂CH₂—, C(O), N(C₁₋₃alkyl), —O— and —N═, wherein each R^{T} is selected from H, F, Me or together with the carbon of C(R^{T})₂ forms a cycloprop-1,1-diyl group;
where X^{G}, X^{H}& X^{J} are selected such that there are not two C(O) groups present at adjacent positions, and that the N-(2,6-dioxopiperidin-3-yl) is not at an adjacent position to either N(C₁₋₃alkyl) or O;
each R^{A} is independently a substituent on any available C atom at X^{A}, X^{B}, X^{C} or X^{D} selected from F, Cl, C₁₋₃alkyl, C₁₋₃alkoxy wherein said C₁₋₃alkyl and C₁₋₃alkoxy is independently optionally substituted by one or more F;
h is 0, 1 or 2; and
wherein the Linker is attached to any C atom at X^{A}, X^{B}, X^{C} or X^{D}.

10. The compound of Formula (I), or a pharmaceutically acceptable salt thereof, according to any one of claims 1 to 4, wherein W is W3 which is: —Z^{B}
where Z^{B} is: 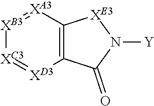
X^{E3} is selected from C(═O) and CR^{E1}R^{E2}, where R^{E1} and R^{E2} are independendently selected from the group consisting of H and C₁₋₃alkyl, or R^{E1} and R^{E2} taken together with the carbons to be they are attached form a C₃₋₆ cycloalkane ring;
Y is N-(2,6-dioxopiperidin-3-yl);
All of X^{A3}, X^{B3}, X^{C3} and X^{D3} are C;
one pair of X^{A3} and X^{B3}, X^{B3} and X^{C3} and X^{C3} and X^{D3} form a five or six membered fully unsaturated or partially unsaturated N containing heterocylic ring, which is attached to the ‘b’ end of the Linker via a N atom;
the others of X^{A3}, X^{B3}, X^{C3} and X^{D3} bear a group RB, where each RB is selected from H, F, Cl, C₁₋₃ alkyl and C₁₋₃ alkoxy.

11. The compound or pharmaceutically acceptable salt thereof, according to any preceding claim wherein R^{N} is H, and p is 0 or 1, R¹ is selected from Cl, F and Me, and R¹, if present, is bound para to Q¹.

12. The compound or pharmaceutically acceptable salt thereof, according to any preceding claim wherein X¹ is N.

13. The compound or pharmaceutically acceptable salt thereof, according to any one of claims 1 to 37 wherein Q¹ is N, Q² is CH and Q³ is CH, and n and m are 1 and 1 or 2 and 0 respectively.

14. The compound or pharmaceutically acceptable salt thereof, according to any preceding claim wherein R^{2a} and R^{2b} are both H.

15. The compound or pharmaceutically acceptable salt thereof, according to any preceding claim wherein:
(a) Q⁴ is a single bond; or
(b) Q⁴ is a NR⁴C(═O) and R⁴ is H.

16. The compound or pharmaceutically acceptable salt thereof according to any preceding claim wherein Y¹, Y², Y³, Y⁴ & Y⁵ are all C.

17. The compound or pharmaceutically acceptable salt thereof according to any preceding claim wherein q is 0 or 1 and R³ (when present) is a substituent on any C atom at Y¹, Y², Y³, Y⁴ & Y⁵ which is F.

18. The compound or pharmaceutically acceptable salt thereof according to any preceding claim wherein Linker is attached at Y⁵.

19. The compound or pharmaceutically acceptable salt thereof according to any one of claims 1 to 34, wherein:
X¹ is N;
p is 1;
R^{N} is H;
n is 2;
m is 0;
R^{2a} and R^{2b} are both H;
Q⁴ is a single bond;
Y¹, Y², Y³ and Y⁴ are all CH;
Y⁵ is C and has the Linker attached; and
q is 0.

20. The compound according to claim 1, selected from:
4-Chloro-7-(4-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-methyl-1H-indole-3-carbonitrile;
4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile;
4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indazole-3-carbonitrile;
4-Chloro-7-[(3S)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile;
4-Chloro-7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indole-3-carbonitrile;
4-Chloro-7-[4-(4-{4-[(4-{2-[(3R*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-7-methoxy-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3R*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-1-oxo-2,3-dihydro-1H-isoindol-5-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-[(3R)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-1H-indole-3-carbonitrile;
7-(4-{4-[4-({1-[2-(2,6-Dioxopiperidin-3-yl)-1-oxo-2,3-dihydro-1H-isoindol-5-yl]piperidin-4-yl}methyl)piperazin-1-yl]phenyl}piperidin-1-yl)-4-fluoro-1H-indole-3-carbonitrile;
7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indole-3-carbonitrile;
7-[(3S)-3-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indole-3-carbonitrile;
4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indazole-3-carbonitrile;
4-Chloro-7-[(3R)-3-{4-[4-({4-[4-(2,4-dioxo-1,3-diazinan-1-yl)-1H-indol-1-yl]piperidin-1-yl}methyl)piperidin-1-yl]phenyl}piperidin-1-yl]-1H-indole-3-carbonitrile;
7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[2-(2,6-dioxopiperidin-3-yl)-1-oxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-4-methyl-1H-indole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-methyl-1H-indole-3-carbonitrile;
7-{4-[4-(4-{4-[2-(2,6-Dioxopiperidin-3-yl)-4-methoxy-3-oxo-2,3-dihydro-1H-pyrrolo[3,4-c]pyridin-6-yl]piperazin-1-yl}butoxy)phenyl]piperidin-1-yl}-4-methyl-1H-indole-3-carbonitrile;
4-Chloro-7-{4-[4-(4-{4-[2-(2,6-dioxopiperidin-3-yl)-4-methoxy-3-oxo-2,3-dihydro-1H-pyrrolo[3,4-c]pyridin-6-yl]piperazin-1-yl}butoxy)phenyl]piperidin-1-yl}-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-4-fluoro-1H-indole-3-carbonitrile;
4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indole-3-carbonitrile;
4-Chloro-7-[4-(4-{4-[(4-{2-[(3R*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
4-Chloro-7-[4-(4-{4-[(4-{2-[(3S*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indole-3-carbonitrile;
4-Chloro-7-[(3S)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile;
4-Chloro-7-[(3S)-3-(4-{4-[(4-{2-[(3R*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
4-Chloro-7-[(3S)-3-(4-{4-[(4-{2-[(3S*)-2,6-dioxopiperidin-3-yl]-4-methoxy-3-oxo-2,3-dihydro-1H-pyrrolo[3,4-c]pyridin-6-yl}piperazin-1-yl)methyl]piperidin-1-yl}phenyl)piperidin-1-yl]-1H-indole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]-2-fluorophenyl}piperidin-1-yl)-4-methyl-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]-2-fluorophenyl}piperidin-1-yl)-4-methyl-1H-indole-3-carbonitrile;
7-(4-{4-[4-({4-[2-(2,6-Dioxopiperidin-3-yl)-7-methoxy-1,3-dioxo-2,3-dihydro-1H-isoindol-5-yl]piperazin-1-yl}methyl)piperidin-1-yl]-2-fluorophenyl}piperidin-1-yl)-4-methyl-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperidin-1-yl}-4-fluoro-1H-indole-3-carbonitrile;
4-Chloro-7-{4-[4-(4-{4-[2-(2,6-dioxopiperidin-3-yl)-1-oxo-2,3-dihydro-1H-isoindol-5-yl]piperazin-1-yl}butoxy)phenyl]piperidin-1-yl}-1H-indole-3-carbonitrile;
4-Chloro-7-{4-[4-(4-{4-[4-(2,4-dioxo-1,3-diazinan-1-yl)-1H-indol-1-yl]piperidin-1-yl}butoxy)phenyl]piperidin-1-yl}-1H-indole-3-carbonitrile;
4-Chloro-7-[(3R)-3-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl]-1H-indole-3-carbonitrile;
7-{4-[4-(4-{4-[2-(2,6-Dioxopiperidin-3-yl)-7-methoxy-1,3-dioxo-2,3-dihydro-1H-isoindol-5-yl]piperazin-1-yl}butoxy)phenyl]piperidin-1-yl}-4-methyl-1H-indole-3-carbonitrile;
4-Chloro-7-(4-{4-[4-({4-[2-(2,6-dioxopiperidin-3-yl)-7-methoxy-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-{4-[4-(4-{4-[2-(2,6-Dioxopiperidin-3-yl)-6-fluoro-1,3-dioxo-2,3-dihydro-1H-isoindol-5-yl]piperazin-1-yl}butoxy)phenyl]piperidin-1-yl}-4-methyl-1H-indole-3-carbonitrile;
4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-(4-{4-[4-({4-[2-(2,6-Dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl)-4-fluoro-1H-indole-3-carbonitrile;
4-Chloro-7-[(3S)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-[(3R)-3-{4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]phenyl}piperidin-1-yl]-1H-indole-3-carbonitrile;
4-Chloro-7-(4-{4-[4-({4-[2-(2,6-dioxopiperidin-3-yl)-1,3-dioxo-2,3-dihydro-1H-isoindol-5-yl]piperazin-1-yl}methyl)piperidin-1-yl]phenyl}piperidin-1-yl)-1H-indole-3-carbonitrile;
7-{4-[4-(4-{4-[2-(2,6-Dioxopiperidin-3-yl)-1,3-dioxo-2,3-dihydro-1H-isoindol-5-yl]piperazin-1-yl}butoxy)phenyl]piperidin-1-yl}-4-methyl-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperidin-1-yl}-1H-indole-3-carbonitrile;
7-{4-[4-(4-{4-[4-(2,4-Dioxo-1,3-diazinan-1-yl)-1H-indol-1-yl]piperidin-1-yl}butoxy)phenyl]piperidin-1-yl}-1H-indole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-1H-indole-3-carbonitrile;
4-Chloro-7-(4-{5-[4-({4-[2-(2,6-dioxopiperidin-3-yl)-7-methoxy-1-oxo-2,3-dihydro-1H-isoindol-5-yl]piperazin-1-yl}methyl)piperidin-1-yl]pyridin-2-yl}piperidin-1-yl)-1H-indole-3-carbonitrile;
4-Chloro-7-(4-{5-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]pyridin-2-yl}piperidin-1-yl)-1H-indole-3-carbonitrile;
4-Chloro-7-(4-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl)-1H-indole-3-carbonitrile;
4-Chloro-7-(4-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl)-1H-indole-3-carbonitrile;
4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridazin-3-yl}piperidin-1-yl)-1H-indole-3-carbonitrile;
4-Chloro-7-[(3R*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile;
4-Chloro-7-[(3S*)-3-{5-[4-({4-[4-(2,4-dioxo-1,3-diazinan-1-yl)-1H-indol-1-yl]piperidin-1-yl}methyl)piperidin-1-yl]pyridin-2-yl}piperidin-1-yl]-1H-indole-3-carbonitrile;
4-Chloro-7-(4-{5-[4-({4-[4-(2,4-dioxo-1,3-diazinan-1-yl)-1H-indol-1-yl]piperidin-1-yl}methyl)piperidin-1-yl]pyrazin-2-yl}piperidin-1-yl)-1H-indole-3-carbonitrile;
4-Chloro-7-(4-{5-[4-({4-[2-(2,6-dioxopiperidin-3-yl)-1-oxo-2,3-dihydro-1H-isoindol-5-yl]piperazin-1-yl}methyl)piperidin-1-yl]pyrazin-2-yl}piperidin-1-yl)-1H-indole-3-carbonitrile;
7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-methyl-1H-indole-3-carbonitrile;
4-Chloro-7-[4-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile;
7-[4-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile;
4-Chloro-7-(4-{4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]phenyl}piperidin-1-yl)-1H-indole-3-carbonitrile;
4-Chloro-7-[(3S)-3-(4-{4-[(4-{[1-(2,6-dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-1H-indole-3-carbonitrile;
7-[(3S)-3-(4-{4-[(4-{[1-(2,6-Dioxopiperidin-3-yl)-3-methyl-2-oxo-2,3-dihydro-1H-benzimidazol-4-yl]methyl}piperazin-1-yl)methyl]piperidin-1-yl}phenyl)piperidin-1-yl]-4-fluoro-1H-indole-3-carbonitrile;
4-Chloro-7-{4-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile;
4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indazole-3-carbonitrile;
4-Chloro-7-{(3S)-3-[4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)phenyl]piperidin-1-yl}-1H-indole-3-carbonitrile;
7-{(3R*)-1-[4-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-{(3S*)-1-[4-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-{(3R*)-1-[3-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-{(3S*)-1-[3-(4-{4-[2-(2,6-dioxopiperidin-3-yl)-1-oxo-2,3-dihydro-1H-isoindol-5-yl]piperazin-1-yl}butoxy)phenyl]piperidin-3-yl}-4-methyl-1H-indole-3-carbonitrile;
7-[(3R*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-4-methyl-1H-indole-3-carbonitrile;
7-[(3S*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-4-methyl-1H-indole-3-carbonitrile;
4-Chloro-7-(4-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl)-1H-indole-3-carbonitrile;
4-Chloro-7-(4-{4-[4-({4-[2-(2,6-dioxopiperidin-3-yl)-1-oxo-2,3-dihydro-1H-isoindol-5-yl]piperazin-1-yl}methyl)piperidine-1-carbonyl]phenyl}piperidin-1-yl)-1H-indole-3-carbonitrile;
4-Chloro-7-{4-[4-(4-{4-[2-(2,6-dioxopiperidin-3-yl)-4-methoxy-3-oxo-2,3-dihydro-1H-pyrrolo[3,4-c]pyridin-6-yl]piperazin-1-yl}piperidin-1-yl)phenyl]piperidin-1-yl}-1H-indole-3-carbonitrile;
4-Chloro-7-(4-{4-[4-(2-{4-[2-(2,6-dioxopiperidin-3-yl)-4-methoxy-3-oxo-2,3-dihydro-1H-pyrrolo[3,4-c]pyridin-6-yl]piperazin-1-yl}ethyl)piperidin-1-yl]phenyl}piperidin-1-yl)-1H-indole-3-carbonitrile;
4-Chloro-7-(4-{4-[4-(3-{4-[2-(2,6-dioxopiperidin-3-yl)-4-methoxy-3-oxo-2,3-dihydro-1H-pyrrolo[3,4-c]pyridin-6-yl]piperazin-1-yl}propyl)piperidin-1-yl]phenyl}piperidin-1-yl)-1H-indole-3-carbonitrile;
7-(4-{4-[4-({4-[5-(2,4-Dioxo-1,3-diazinan-1-yl)-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[4-({4-[5-(2,4-dioxo-1,3-diazinan-1-yl)-1H-indol-1-yl]piperidin-1-yl}methyl)piperidin-1-yl]phenyl}piperidin-1-yl)-1H-indole-3-carbonitrile;
4-Chloro-7-[(3R*)-1-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-3-yl]-1H-indole-3-carbonitrile;
4-Chloro-7-[(3S*)-1-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-3-yl]-1H-indole-3-carbonitrile;
4-Chloro-7-[(3R*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-1H-indole-3-carbonitrile;
4-Chloro-7-[(3S*)-1-{4-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-3-yl]-1H-indole-3-carbonitrile;
7-{4-[4-(4-{4-[2-(2,6-Dioxopiperidin-3-yl)-1-oxo-2,3-dihydro-1H-isoindol-5-yl]piperazin-1-yl}butoxy)phenyl]piperazin-1-yl}-4-methyl-1H-indole-3-carbonitrile;
4-Chloro-7-[(3R*)-1-{4-[1-({1-[2-(2,6-dioxopiperidin-3-yl)-1-oxo-2,3-dihydro-1H-isoindol-5-yl]piperidin-4-yl}methyl)piperidin-4-yl]phenyl}piperidin-3-yl]-1H-indole-3-carbonitrile;
4-Chloro-7-[(3S*)-1-{4-[1-({1-[2-(2,6-dioxopiperidin-3-yl)-1-oxo-2,3-dihydro-1H-isoindol-5-yl]piperidin-4-yl}methyl)piperidin-4-yl]phenyl}piperidin-3-yl]-1H-indole-3-carbonitrile;
4-Chloro-7-[(3R*)-1-{4-[1-({1-[2-(2,6-dioxopiperidin-3-yl)-4-methoxy-3-oxo-2,3-dihydro-1H-pyrrolo[3,4-c]pyridin-6-yl]piperidin-4-yl}methyl)piperidin-4-yl]phenyl}piperidin-3-yl]-1H-indole-3-carbonitrile;
4-Chloro-7-[(3S*)-1-{4-[1-({1-[2-(2,6-dioxopiperidin-3-yl)-4-methoxy-3-oxo-2,3-dihydro-1H-pyrrolo[3,4-c]pyridin-6-yl]piperidin-4-yl}methyl)piperidin-4-yl]phenyl}piperidin-3-yl]-1H-indole-3-carbonitrile;
4-Chloro-7-(4-{4-[4-({4-[2-(2,6-dioxopiperidin-3-yl)-6-fluoro-1,3-dioxo-2,3-dihydro-1H-isoindol-5-yl]piperazin-1-yl}methyl)piperidine-1-carbonyl]phenyl}piperidin-1-yl)-1H-indole-3-carbonitrile;
7-[4-(4-{[1-({1-[2-(2,6-Dioxopiperidin-3-yl)-1-oxo-2,3-dihydro-1H-isoindol-5-yl]piperidin-4-yl}methyl)piperidin-4-yl]oxy}phenyl)piperidin-1-yl]-4-fluoro-1H-indole-3-carbonitrile;
4-Chloro-7-(4-{4-[4-({4-[5-(2,4-dioxo-1,3-diazinan-1-yl)-7-methyl-1H-indol-1-yl]piperidin-1-yl}methyl)piperidin-1-yl]phenyl}piperidin-1-yl)-1H-indole-3-carbonitrile;
4-Chloro-7-(4-{4-[4-({4-[6-(2,4-dioxo-1,3-diazinan-1-yl)-1-methyl-1H-pyrrolo[3,2-b]pyridin-2-yl]piperidin-1-yl}methyl)piperidin-1-yl]phenyl}piperidin-1-yl)-1H-indole-3-carbonitrile;
4-{4-[(1-{4-[(3S)-1-(4-chloro-3-cyano-1H-indol-7-yl)piperidin-3-yl]phenyl}piperidin-4-yl)methyl]piperazin-1-yl}-N-(2,6-dioxopiperidin-3-yl)-2-fluorobenzamide;
4-{4-[(1-{4-[1-(3-Cyano-4-fluoro-1H-indol-7-yl)piperidin-4-yl]phenyl}piperidin-4-yl)methyl]piperazin-1-yl}-N-(2,6-dioxopiperidin-3-yl)-2-fluorobenzamide;
4-{4-[(1-{4-[1-(4-Chloro-3-cyano-1H-indol-7-yl)piperidin-4-yl]phenyl}piperidin-4-yl)methyl]piperazin-1-yl}-N-(2,6-dioxopiperidin-3-yl)-2-fluorobenzamide;
7-[4-(4-{[4-({1-[2-(2,6-Dioxopiperidin-3-yl)-1-oxo-2,3-dihydro-1H-isoindol-5-yl]piperidin-4-yl}methyl)piperazin-1-yl]methyl}phenyl)piperidin-1-yl]-4-fluoro-1H-indole-3-carbonitrile;
4-Chloro-7-(4-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl)-1H-indole-3-carbonitrile;
4-Chloro-7-(4-{6-[4-({4-[2-(2,6-dioxopiperidin-3-yl)-1-oxo-2,3-dihydro-1H-isoindol-5-yl]piperazin-1-yl}methyl)piperidin-1-yl]pyridin-3-yl}piperidin-1-yl)-1H-indole-3-carbonitrile;
4-Chloro-7-{4-[4-(4-{4-[2-(2,6-dioxopiperidin-3-yl)-1-oxo-2,3-dihydro-1H-isoindol-5-yl]piperazin-1-yl}piperidin-1-yl)phenyl]piperidin-1-yl}-1H-indole-3-carbonitrile;
4-Chloro-7-[4-(4-{4-[4-(2,4-dioxo-1,3-diazinan-1-yl)-1H-indol-1-yl][1,4′-bipiperidin]-1′-yl}phenyl)piperidin-1-yl]-1H-indole-3-carbonitrile;
4-chloro-7-(4-{4-[4-({4-[2-(2,6-dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-1-methyl-1H-indole-3-carbonitrile;
4-Chloro-7-(4-{3-[4-({4-[2-(2,6-dioxopiperidin-3-yl)-1-oxo-2,3-dihydro-1H-isoindol-5-yl]piperazin-1-yl}methyl)piperidin-1-yl]phenyl}piperidin-1-yl)-1H-indole-3-carbonitrile;
4-Chloro-7-[(3S)-3-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl]-1H-indole-3-carbonitrile;
4-Chloro-7-[(3S*)-3-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl]-1H-indole-3-carbonitrile;
4-Chloro-7-[(3R*)-3-{6-[4-({4-[4-(2,4-dioxo-1,3-diazinan-1-yl)-1H-indol-1-yl]piperidin-1-yl}methyl)piperidin-1-yl]pyridin-3-yl}piperidin-1-yl]-1H-indole-3-carbonitrile;
4-Chloro-7-[(2S*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indole-3-carbonitrile;
4-Chloro-7-[(2R*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indole-3-carbonitrile;
4-Chloro-7-[(2S*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indazole-3-carbonitrile;
4-Chloro-7-[(2R*)-2-{4-[4-({4-[4-(2,4-dioxo-1,3-diazinan-1-yl)-1H-indol-1-yl]piperidin-1-yl}methyl)piperidin-1-yl]phenyl}morpholin-4-yl]-1H-indazole-3-carbonitrile;
4-Chloro-7-[(3R*)-3-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl]-1H-indole-3-carbonitrile;
4-Chloro-7-[(3S*)-3-{6-[4-({4-[4-(2,4-dioxo-1,3-diazinan-1-yl)-1H-indol-1-yl]piperidin-1-yl}methyl)piperidin-1-yl]pyridazin-3-yl}piperidin-1-yl]-1H-indole-3-carbonitrile;
4-Chloro-7-(4-{4-[4-({4-[4-(2,4-dioxo-1,3-diazinan-1-yl)-2-methyl-1H-indol-1-yl]piperidin-1-yl}methyl)piperidin-1-yl]phenyl}piperidin-1-yl)-1H-indole-3-carbonitrile;
4-Chloro-7-(4-{2-[4-({4-[2-(2,6-dioxopiperidin-3-yl)-1-oxo-2,3-dihydro-1H-isoindol-5-yl]piperazin-1-yl}methyl)piperidin-1-yl]pyrimidin-5-yl}piperidin-1-yl)-1H-indole-3-carbonitrile;
4-Chloro-7-(4-{2-[4-({4-[4-(2,4-dioxo-1,3-diazinan-1-yl)-1H-indol-1-yl]piperidin-1-yl}methyl)piperidin-1-yl]pyrimidin-5-yl}piperidin-1-yl)-1H-indole-3-carbonitrile;
7-(4-{4-[(2R)-2-({4-[2-(2,6-dioxopiperidin-3-yl)-1-oxo-2,3-dihydro-1H-isoindol-5-yl]piperazin-1-yl}methyl)morpholin-4-yl]phenyl}piperidin-1-yl)-4-fluoro-1H-indole-3-carbonitrile;
4-Chloro-7-(4-{4-[(2R)-2-({4-[2-(2,6-dioxopiperidin-3-yl)-1-oxo-2,3-dihydro-1H-isoindol-5-yl]piperazin-1-yl}methyl)morpholin-4-yl]phenyl}piperidin-1-yl)-1H-indole-3-carbonitrile;
7-(4-{4-[7-({4-[2-(2,6-Dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl)-4-fluoro-1H-indole-3-carbonitrile;
4-Chloro-7-(4-{4-[7-({4-[2-(2,6-dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl)-1H-indole-3-carbonitrile;
4-Chloro-7-[(3S)-3-{4-[7-({4-[2-(2,6-dioxopiperidin-3-yl)-1-oxo-2,3-dihydro-1H-isoindol-5-yl]piperazin-1-yl}methyl)-2-azaspiro[3.5]nonan-2-yl]phenyl}piperidin-1-yl]-1H-indole-3-carbonitrile;
4-Chloro-7-[(3S)-3-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl]-1H-indole-3-carbonitrile;
4-Chloro-7-[(3R)-3-{4-[4-({4-[6-(2,4-dioxo-1,3-diazinan-1-yl)-1-methyl-1H-indol-2-yl]piperidin-1-yl}methyl)piperidin-1-yl]phenyl}piperidin-1-yl]-1H-indazole-3-carbonitrile;
7-[(3S)-3-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl]-4-fluoro-1H-indazole-3-carbonitrile;
7-[(3S)-3-{4-[4-({4-[4-(2,4-Dioxo-1,3-diazinan-1-yl)-1H-indol-1-yl]piperidin-1-yl}methyl)piperidin-1-yl]phenyl}piperidin-1-yl]-4-fluoro-1H-indazole-3-carbonitrile;
7-(4-{4-[7-({4-[4-(2,4-Dioxo-1,3-diazinan-1-yl)-1H-indol-1-yl]piperidin-1-yl}methyl)-2-azaspiro[3.5]nonan-2-yl]phenyl}piperidin-1-yl)-4-fluoro-1H-indole-3-carbonitrile;
4-Chloro-7-[(3R)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indole-3-carbonitrile;
4-Chloro-7-[(3S)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indole-3-carbonitrile;
4-Chloro-7-[(3R)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indazole-3-carbonitrile;
4-Chloro-7-[(3S)-3-{4-[4-({4-[6-(2,4-dioxo-1,3-diazinan-1-yl)-5-fluoro-1-methyl-1H-indol-2-yl]piperidin-1-yl}methyl)piperidin-1-yl]phenyl}piperidin-1-yl]-1H-indazole-3-carbonitrile;
7-(4-{4-[4-({4-[6-(2,4-Dioxo-1,3-diazinan-1-yl)-1-methyl-1H-indol-2-yl]piperidin-1-yl}methyl)piperidin-1-yl]phenyl}piperidin-1-yl)-4-fluoro-1H-indazole-3-carbonitrile;
7-(4-{4-[4-({4-[4-(2,4-Dioxo-1,3-diazinan-1-yl)-2-methyl-1H-indol-1-yl]piperidin-1-yl}methyl)piperidin-1-yl]phenyl}piperidin-1-yl)-4-fluoro-1H-indole-3-carbonitrile;
4-Chloro-7-(4-{4-[7-({4-[4-(2,4-dioxo-1,3-diazinan-1-yl)-1H-indol-1-yl]piperidin-1-yl}methyl)-2-azaspiro[3.5]nonan-2-yl]phenyl}piperidin-1-yl)-1H-indole-3-carbonitrile;
4-Chloro-7-(4-{4-[4-({4-[2-(2,6-dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-1H-indazole-3-carbonitrile;
7-(4-{4-[4-({4-[2-(2,6-Dioxopiperidin-3-yl)-4-methoxy-3-oxo-2,3-dihydro-1H-pyrrolo[3,4-c]pyridin-6-yl]piperazin-1-yl}methyl)piperidin-1-yl]phenyl}piperidin-1-yl)-4-fluoro-1H-indazole-3-carbonitrile;
7-(4-{4-[4-({4-[2-(2,6-Dioxopiperidin-3-yl)-1-oxo-2,3-dihydro-1H-isoindol-5-yl]piperazin-1-yl}methyl)piperidin-1-yl]phenyl}piperidin-1-yl)-4-fluoro-1H-indazole-3-carbonitrile;
4-Chloro-7-(4-{4-[4-({4-[4-(2,4-dioxo-1,3-diazinan-1-yl)-1H-indol-1-yl]piperidin-1-yl}methyl)piperidin-1-yl]phenyl}piperidin-1-yl)-1H-indazole-3-carbonitrile;
7-(4-{4-[4-({4-[4-(2,4-Dioxo-1,3-diazinan-1-yl)-1H-indol-1-yl]piperidin-1-yl}methyl)piperidin-1-yl]phenyl}piperidin-1-yl)-4-fluoro-1H-indazole-3-carbonitrile;
7-{4-[4-(4-{4-[2-(2,6-Dioxopiperidin-3-yl)-7-methoxy-1-oxo-2,3-dihydro-1H-isoindol-5-yl]piperazin-1-yl}butoxy)phenyl]piperidin-1-yl}-4-methyl-1H-indazole-3-carbonitrile;
7-(4-{4-[4-({4-[2-(2,6-Dioxopiperidin-3-yl)-7-methoxy-1-oxo-2,3-dihydro-1H-isoindol-5-yl]piperazin-1-yl}methyl)piperidin-1-yl]phenyl}piperidin-1-yl)-4-methyl-1H-indazole-3-carbonitrile;
7-{(3S*)-3-[4-(4-{4-[2-(2,6-Dioxopiperidin-3-yl)-1-oxo-2,3-dihydro-1H-isoindol-5-yl]piperazin-1-yl}butoxy)phenyl]pyrrolidin-1-yl}-4-methyl-1H-indole-3-carbonitrile;
7-{(3R*)-3-[4-(4-{4-[2-(2,6-Dioxopiperidin-3-yl)-1-oxo-2,3-dihydro-1H-isoindol-5-yl]piperazin-1-yl}butoxy)phenyl]pyrrolidin-1-yl}-4-methyl-1H-indole-3-carbonitrile;
N-[1-(4-Chloro-3-cyano-1H-indazol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[5-(2,4-dioxo-1,3-diazinan-1-yl)-1H-indol-1-yl]piperidin-1-yl}methyl)piperidin-1-yl]-2-fluorobenzamide;
N-[1-(4-chloro-3-cyano-1~{H}-indol-7-yl)-4-piperidyl]-4-[4-[[4-[4-[(2,6-dioxo-3-piperidyl)carbamoyl]-3-fluoro-phenyl]piperazin-1-yl]methyl]-1-piperidyl]-2-fluorobenzamide;
N-[1-(4-Chloro-3-cyano-1H-indol-7-yl)piperidin-4-yl]-4-(4-{[6-(2,6-dioxopiperidin-3-yl)-5,7-dioxo-3,5,6,7-tetrahydropyrrolo[3,4-f]isoindol-2(1H)-yl]methyl}piperidin-1-yl)-2-fluorobenzamide;
N-[1-(4-Chloro-3-cyano-1H-indol-7-yl)piperidin-4-yl]-4-[4-({4-[6-(2,4-dioxo-1,3-diazinan-1-yl)-5-fluoro-1-methyl-1H-indol-2-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[1-(2,6-dioxopiperidin-3-yl)-3-methyl-2-oxo-2,3-dihydro-1H-benzimidazol-4-yl]piperazin-1-yl}methyl)piperidin-1-yl]-2-fluorobenzamide;
N-[1-(4-Chloro-3-cyano-1H-indol-7-yl)piperidin-4-yl]-4-[4-({4-[6-(2,4-dioxo-1,3-diazinan-1-yl)-1-methyl-1H-indol-2-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[2-(2,6-dioxopiperidin-3-yl)-1-oxo-2,3-dihydro-1H-isoindol-5-yl]piperazin-1-yl}methyl)piperidin-1-yl]-2-fluorobenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]-2-fluorobenzamide;
N-[1-(4-Chloro-3-cyano-1H-indol-7-yl)piperidin-4-yl]-2-cyano-4-[4-({4-[4-(2,4-dioxo-1,3-diazinan-1-yl)-1H-indol-1-yl]piperidin-1-yl}methyl)piperidin-1-yl]benzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-fluoro-6-methylbenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-3-methylbenzamide;
N-[1-(4-Chloro-3-cyano-1H-indol-7-yl)piperidin-4-yl]-3-cyano-4-[4-({4-[4-(2,4-dioxo-1,3-diazinan-1-yl)-1H-indol-1-yl]piperidin-1-yl}methyl)piperidin-1-yl]benzamide;
N-[1-(4-Chloro-3-cyano-1H-indol-7-yl)piperidin-4-yl]-5-(4-{4-[2-(2,6-dioxopiperidin-3-yl)-6-fluoro-1,3-dioxo-2,3-dihydro-1H-isoindol-5-yl]piperazin-1-yl}piperidin-1-yl)pyridine-2-carboxamide;
N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyridine-2-carboxamide;
N-[1-(4-Chloro-3-cyano-1H-indol-7-yl)piperidin-4-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide;
N-[1-(4-Chloro-3-cyano-1H-indol-7-yl)piperidin-4-yl]-5-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]pyridine-2-carboxamide;
N-[1-(4-Chloro-3-cyano-1H-indazol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyridine-2-carboxamide;
N-[1-(3-Cyano-4-methyl-1H-indol-7-yl)piperidin-4-yl]-5-[4-({4-[2-(2,6-dioxopiperidin-3-yl)-6-fluoro-1,3-dioxo-2,3-dihydro-1H-isoindol-5-yl]piperazin-1-yl}methyl)piperidin-1-yl]pyridine-2-carboxamide;
N-[1-(3-Cyano-4-methyl-1H-indol-7-yl)piperidin-4-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]benzamide;
N-[4-(4-Chloro-3-cyano-1H-indol-7-yl)cyclohexyl]-4-[4-({4-[4-(2,4-dioxo-1,3-diazinan-1-yl)-1H-indol-1-yl]piperidin-1-yl}methyl)piperidin-1-yl]-2-fluorobenzamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2,6-difluorobenzamide;
N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyrazine-2-carboxamide;
N-[1-(4-Chloro-3-cyano-1H-indol-7-yl)piperidin-4-yl]-5-[4-({4-[4-(2,4-dioxo-1,3-diazinan-1-yl)-1H-indol-1-yl]piperidin-1-yl}methyl)piperidin-1-yl]pyrimidine-2-carboxamide;
N-[1-(4-Chloro-3-cyano-1H-indol-7-yl)piperidin-4-yl]-4-[4-({4-[4-(2,4-dioxo-1,3-diazinan-1-yl)-1H-indol-1-yl]piperidin-1-yl}methyl)piperidin-1-yl]-2-methylbenzamide;
N-[1-(4-Chloro-3-cyano-1H-indol-7-yl)piperidin-4-yl]-6-[4-({4-[4-(2,4-dioxo-1,3-diazinan-1-yl)-1H-indol-1-yl]piperidin-1-yl}methyl)piperidin-1-yl]pyridazine-3-carboxamide;
N-[(3R*)-1-(4-Chloro-3-cyano-1H-indol-7-yl)piperidin-3-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide; and
N-[(3S*)-1-(4-Chloro-3-cyano-1H-indol-7-yl)piperidin-3-yl]-5-[4-({4-[2-(2,6-dioxopiperidin-3-yl)-1-oxo-2,3-dihydro-1H-isoindol-5-yl]piperazin-1-yl}methyl)piperidin-1-yl]pyridine-2-carboxamide;
or pharmaceutically acceptable salts thereof.

21. A pharmaceutical composition, which comprises a compound of Formula (I) or PROTAC of Formula (Ia), or a pharmaceutically acceptable salt thereof according to one of claims 1-20, in association with a pharmaceutically acceptable excipient.

22. The compound of Formula (I) or PROTAC of Formula (Ia), or a pharmaceutically acceptable salt thereof, according to any one of claims 1-20, for use in a method of treatment of the human or animal body by therapy.

23. A compound of Formula (I) or PROTAC of Formula (Ia), or a pharmaceutically acceptable salt thereof, according to any one of claims 1-20, for use as an anti-invasive agent in the containment and/or treatment of solid tumour disease.

24. Use of a compound of Formula (I) or PROTAC of Formula (Ia), or a pharmaceutically acceptable salt thereof, according to any one of claims 1-20, for the manufacture of a medicament for use as an anti-invasive agent in the containment and/or treatment of solid tumour disease.

25. A method for producing an anti-invasive effect by the containment and/or treatment of solid tumour disease, in a warm-blooded animal, such as man, in need of such effect, which comprises administering to said animal an effective amount of a compound of Formula (I) or PROTAC of Formula (Ta), or a pharmaceutically acceptable salt thereof, according to any one of claims 1-20.
